(19) United States
(12) Patent Application Publication
(10) Pub. No.: US 2004/0110802 A1
(43) Pub. Date: Jun. 10, 2004

(54) Antibacterial benzoic acid derivatives

(76) Inventors: Atli Thorarensen, O'Fallon, MO (US); J. Craig Ruble, Greenwood, IN (US); Jed F. Fisher, Kalamazoo, MI (US); Donna Lee Romero, Chesterfield, MO (US); Thomas J. Beauchamp, Noblesville, IN (US); Jill M. Northuis, Portage, MI (US)

Correspondence Address: PHARMACIA & UPJOHN, 301 HENRIETTA ST, 0228-32-LAW, KALAMAZOO, MI, 49007, US

(21) Appl. No.: 10/645,802

(22) Filed: Aug. 20, 2003

Related U.S. Application Data
(60) Provisional application No. 60/405,429, filed on Aug. 23, 2002.
(60) Provisional application No. 60/430,592, filed on Dec. 3, 2002.

(51) Int. Cl. A61K 31/44; C07D 213/57; C07D 213/46

(52) U.S. Cl. 514/355; 514/357; 546/315; 546/330; 546/313

(57) ABSTRACT

The invention provides antimicrobial agents and methods of using the agents for sterilization, sanitation, antisepsis, disinfection, and treatment of infections in mammals.

### CROSS-REFERENCE

This application claims the benefit of U.S. Ser. Nos. 60/405,429 and 60/430,592 filed Aug. 23, 2002 and Dec. 3, 2002 respectively, under 35 USC 119(e)(i).

### FIELD OF THE INVENTION

The present invention relates to antibacterial agents that are useful for sterilization, sanitation, antisepsis, and disinfection.

### BACKGROUND

The inappropriate growth of a variety of bacteria has been a problem for many years. Bacteria have caused degradation of natural product materials, infection in humans and other animals, and spoilage of foods.

Sterilization denotes the use of either physical or chemical agents to eliminate all viable bacteria from a material, while disinfection generally refers to the use of germicidal chemical agents to destroy the potential infectivity of a material. Sanitizing refers to procedures used to simply lower the bacterial content of utensils used for food. Antisepsis refers to the topical application of chemicals to a body surface to kill or inhibit pathogenic microbes. Disinfectants are widely used for skin antisepsis in preparation for surgery.

Bacteria are the smallest organisms that contain all the machinery required for growth and self-replication. A bacterium includes a rigid cell wall surrounding the cytoplasmic membrane, which itself encloses a single naked chromosome without a nuclear membrane. The cytoplasmic membrane consists primarily of a bi-layer of lipid molecules.

The fundamental criterion of bactericidal action is loss of the ability of the organism to propagate indefinitely, when placed in a suitable environment. Bactericidal action suggests microbe damage of various types, including the triggering of irreversible damage to the cytoplasmic cell membrane or irreversible impairment of the DNA (or viral RNA replication. Accordingly, sterilization is not identical with destruction of microbes. Additionally, it is understood that damage to nucleic acids (DNA or RNA) is not always irreversible, as it is known that ultraviolet light-induced damage to viral nucleic acids can be repaired by enzymatic and genetic mechanisms.

### SUMMARY OF THE INVENTION

The invention relates to antibacterial agents that are useful for sterilization, sanitation, antisepsis, and disinfection.

In one aspect, the invention features methods of using antibacterial agents of formula I for sterilizing, sanitizing, antisepsis, or disinfecting. The method includes applying the antibacterial agent to a location in need of sterilization, sanitation, antisepsis, and disinfection.

In general, the antibacterial agents have the formula

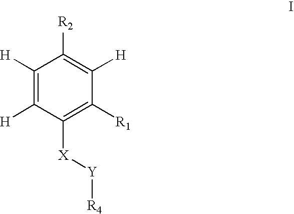

wherein

X═NH

Y═CO, CS, —C(═N—CN) or

X and Y together form an alkene, or cycloalkyl;

R₁ is —COOH;

R₂ is an electron withdrawing group; and

R₄ is an optionally substituted HET.

### DETAILED DESCRIPTION OF THE INVENTION

The term “halo” refers to a halogen atom selected from Cl, Br, I, and F.

The term “alkyl” refers to both straight- and branched-chain moieties. Unless otherwise specifically stated alkyl moieties include between 1 and 9 carbon atoms.

The term “alkenyl” refers to both straight- and branched-chain moieties containing at least one —C≡C—. Unless otherwise specifically stated alkenyl moieties include between 1 and 9 carbon atoms.

The term “alkynyl” refers to both straight- and branched-chain moieties containing at least one —C≡C—. Unless otherwise specifically stated alkynyl moieties include between 1 and 9 carbon atoms. between 1 and 6 carbon atoms

The term “alkoxy” refers to -O-alkyl groups.

The term “cycloalkyl” refers to a cyclic alkyl moiety. Unless otherwise specifically stated cycloalkyl moieties will include between 3 and 9 carbon atoms.

The term “cycloalkenyl” refers to a cyclic alkenyl moiety. Unless otherwise specifically stated cycloalkyl moieties will include between 3 and 9 carbon atoms and at least one —C≡C— group within the cyclic ring.

The term “amino” refers to —NH₂.

The term “aryl” refers to phenyl and naphthyl.

The term “het” refers to mono- or bi-cyclic ring systems containing at least one heteroatom selected from O, S, and N. Each mono-cyclic ring may be aromatic, saturated, or partially unsaturated. A bi-cyclic ring system may include a mono-cyclic ring containing at least one heteroatom fuised with an cycloalkyl or aryl group. A bicyclic ring system may also include a mono-cyclic ring containing at least one heteroatom fuised with another het, mono-cyclic ring system.

Examples of “het” include, but are not limited to, pyridine, thiophene, furan, pyrazoline, pyrimidine, 2-pyridyl, 3-pyridyl, 4-pyridyl, 2-pyrimidinyl, ⁴-pyrimidinyl, 5-pyrimidinyl, 3-pyridazinyl, 4-pyridazinyl, 3-pyrazinyl, 4-oxo-2-imidazolyl, 2-imidazolyl, 4-imnidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-ffiranyl, 3-ffiranyl, 2-thienyl, 3-thienyl, 2-pyrrolyl, 3-pyrrolyl, 3-isopyrrolyl, 4-isopyrrolyl, 5-isopyrrolyl, 1,2,3 ,-oxathiazole-1-oxide, 1,2,4-oxadiazol-3-yl, 1,2,4-oxadiazol-5-yl, 5-oxo-1,2,4oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2. 1]heptyl.

The term “heteroaryl” refers to a mono- or bicylic het in which at least one cyclic ring is aromatic.

The term “substituted alkyl” refers to an alkyl moiety including 1-4 substituents selected from halo, het, cycloalkyl, cycloalkenyl, aryl, —OQ₁₀, —SQ₁₀, —S(O)₂Q₁₀, —S(O)Q₁₀, —OS(O)2Q₁₀, —C(═NQ₁₀)Q₁₀, —C(═N—O-Q₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(S)NQ₁₀Q₁₀, —N(Q₁₀)C(S)NQ₁₀Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, Each of the het, cycloalkyl, cycloalkenyl, and aryl being optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, —NQ₁₀C(O)Q₁₀, —N(Q₁₀)C(S)NQ₁₀Q₁₀, —N(Q₁₀)C(S)Q₁₀, —NQ₁₀C(O)NQ₁₀Q₁₀, —S(O)₂NQ₁₀Q₁₀, —NQ₁₀S(O)₂Q₁₀, -NQ₁₀S(O)Q₁₀, —NQ₁₀SQ₁₀, —NO₂, —SNQ₁₀Q₁₀, alkyl, substituted alkyl, alkenyl, alkynyl, het, halo, cycloalkyl, cycloalkenyl, and aryl. The het, cycloalkyl, cycloalkenyl, alkenyl, alkynyl, and aryl being optionally substituted with 1-3 substitutuents selected from halo and Q₁₅.

The term “substituted het” refers to a het moiety including 1-4 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(QI₆)₂OC(O)Q₁₀, —CN, ═O, =S, —NQ₁₀C(O)Q₁₀, —NQ₁₀C(S)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O) —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted alkenyl” refers to a alkenyl moiety including 1-3 substituents —OQ₁₀, —SQ₁₀, —S(O)₂Q1O, —S(O)Q1O, —OS(O)₂Q1O, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(QI₆)₂OC(O)Q₁₀, —CN, ═O, =S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂—NQ₁₀S(O)Q₁₀, —NQ₁₀SQ₁₀, —NO₂, —SNQ₁₀Q₁₀, allyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, =S, —NQIoC(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted cycloalkenyl” refers to a cycloalkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, =S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted amino” refers to an amino moiety in which one or both of the amino hydrogens are replaced with a group selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂N═S(O —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, =S, —NQIOC(O)Q₁₀, —NQ₁₀C(S)Q₁₀, —NQ₁₀C(O)NQ₁₀Q₁₀, —NQ₁₀C(S)NQ₁₀Q₁₀, —S(O)₂NQ₁₀Q_{—NQ}₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

Each Q₁₀ is independently selected from —H, alkyl, cycloalkyl, het, cycloalkenyl, and aryl. The het, cycloalkyl, cycloalkenyl, and aryl being optionally substituted with 1-3 substitutuents selected from halo and Q₁₃.

Each Q₁₁ is independently selected from —H, halo, alkyl, aryl, cycloalkyl, and het. The alkyl, aryl, cycloalkyl, and het being optionally substituted with 1-3 substituents independently selected from halo, —NO₂, —CN, ═S, ═O, and Q₁₄.

Each Q₁₃ is independently selected from Q₁₁, —OQ₁₁, —SQ₁₁, —S(O)₂Q₁, —S(O)Q₁₁, —OS(O)₂Q₁₁, —C(═NQ₁₁)Q₁₁, —S(O)₂—N═S(O)(Q₁₁)₂, —S(O)₂—N═S(Q₁₁) —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —C(S)NQ,,Q₁₁, —C(O)C(Q₁₆)₂OC(O)Q₁₀, —CN, ═O, ═S, —NQ,,C(O)Q₁₁, —NQ,,C(S)Q₁₁, —NQ₁₁C(O)NQ₁₁Q₁₁, —NQ₁₁C(S)NQ₁₁Q₁₁, —S(O)₂NQ₁₁Q₁₁, —NQ₁₁S(O)₂Q₁₁, —NQ₁₁S(O)Q₁₁, —NQ₁₁SQ₁₁, —NO₂, and —SNQ₁₁Q₁₁.

Each Q₁₄ is —H or a substituent selected from alkyl, cycloalkyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —C(S)NQ₁₆Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, and —NQ₁₆S(O)₂Q₁₆. The alkyl, cycloalkyl, and cycloalkenyl being furher optionally substituted with ═O or ═S.

Each Q₁₅ is H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ16Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being furher optionally substituted with ═O or ═S.

Each Q₁₆ is independently selected from —H, allyl, and cycloalkyl. The alkyl and cycloalkyl optionally including 1-3 halos. Mammal denotes human and animals.

Each Q₁₇ is independently selected from —H, —OH, and alkyl optionally including 1-3 halos and —OH.

The term “electron withdrawing group” refers to the ability of a substituent to withdraw electrons relative to that of hydrogen if the hydrogen atom occupied the same position on the molecule. The term “electron withdrawing group” is well understood by one skilled in the art and is discussed in Advanced Organic Chemistry by J. March, John Wiley & Sons, New York, N.Y., (1985) and the discussion therein is incorporated herein by reference. Electron withdrawing groups include, but are not limited to, groups such as halo, nitro, carboxy, cyano, aryl optionally substituted, aromatic het (excluding pyridine) optionally substituted, —OC(A)₃, —C(Zₙ)₃, —C(Zₙ)₂—O—C(Zₘ)₃, —(CO)-Q₁₇, —SO₂—C(Zₘ)₃, —SO₂-aryl, —C(NQ₁₇)Q₁₇, —CH═C(Q₁₇)₂, —C≡C-Q₁₇ in which each Zn and Zm is independently H, halo, —CN, —NO₂ —OH, or C₁₋₄alkyl optionally substituted with 1-3 halo, —OH, N0₂, and provided that at least one of Zn is halo, —CN, or N0₂, and further provided that Q17 is not —OH when the the electron withdrawing group is CO)-Q₁₇.

It is to be understood that the present invention encompasses any racemic, optically-active, polymorphic, tautomeric, or stereoisomeric form, or mixture thereof, of a compound of the invention, which possesses the useful properties described herein.

In cases where compounds are sufficiently basic or acidic to form stable nontoxic acid or base salts, use of the compounds as pharmaceutically acceptable salts may be appropriate. Examples of pharmaceutically acceptable salts which are within the scope of the present invention include organic acid addition salts formed with acids which form a physiological acceptable anion and inorganic salts. Examples of pharmaceutically acceptable salts include, but are not limited to, the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, suiliric, tannic, tartaric, teoclic toluenesulfonic, primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyarrine resins, procaine, purines, theobromine, triethylamine, trimethylamine, tripropylamine, and the like.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA.

The antibacterial agents described herein are useful for sterilization, sanitation, antisepsis, and disinfection. The antibacterial agents can be applied to a location in need of sterilization, sanitation, antisepsis, or disinfection, by methods known to those skilled in the art. For instance, the antibacterial agents may be incorporated into a cleaning solution that is applied, such as by spraying or pouring, to an item in need of sterilization, sanitation, antisepsis, or disinfection. The antibacterial agents may be used alone or in combination, e.g., agents disclosed herein with one another or agent(s) disclosed herein with other antibacterial agents. The antibacterial agents may be applied in varying concentrations depending upon the bacterial susceptibility to antibacterial agent(s) being applied and the desired level of sterilization, sanitation, antisepsis, or disinfection.

In other embodiments, certain antibacterial agents described herein are useful for treating microbial infections in mammals, such as by administering an effective amount of the antibacterial agent compound to the mammal.

Examples of compounds useful as antimicrobial agents for the treatment of microbial infections mammas, include, but are not limited to,

5-bromo-2-({[3-chloro-5-(trifluoromethyl)-2-pyridinyl]carbonyl} aniano)benzoic acid

2-({[4-({[2-(4-aminophenyl) ethyl] amino} sulfonyl)-3-methylthien-2yl]carbonyl}amino)-5-bromobenzoic acid

5-bromo-2-({[4-({[3-(1H-imidazol-1-yl)propyl]amino]sulfonyl)-3-methylthien-2-yl]carbonyl}amino]benzoic acid

5-bromo-2-({[5-({4-[3-(trifluoromethyl)phenyl]piperazin-1-yl}sulfonyl)thien-3yl]carbonyl}amino)benzoic acid

5-bromo-2-({[3-methyl-4-({4-[3-(trifluoromethyl)phenyl]piperazin-1-yl}sulfonyl)thien-2-yl]carbonyl}amino)benzoic acid

5-bromo-2-({[5-methyl-4-({4-[3-(trifluoromethyl)phenyl]piperazin-1-yl}sulfonyl)thien-2-yl]carbonyl}amino)benzoic acid

2-{[(4-{[(1-benzylpiperidin-4-yl)amino]sulfonyl}-3-methylthien-2-yl)carbonyl]amino}5-bromobenzoic acid

2-{[(4-{[(1-benzylpiperidin-4-yl)amino]sulfonyl}-5-methylthien-2-yl)carbonyl]amino}5-bromobenzoic acid

5-bromo-2-({[5-({[3-(dibutylamino)propyl]amino}sulfonyl)thien-3yl]carbonyl} amino)benzoic acid

5-bromo-2-({[4-({[3-(dibutylamino)propyl]amino}sulfonyl)-3-methylthien-2yl]carbonyl}amino)benzoic acid

5-bromo-2-({[4-({[3-(dibutylamino)propyl]amino}sulfonyl)-5-methylthien-2yl]carbonyl}amino)benzoic acid

5-chloro-2-({[5-chloro-2-(methylsulfonyl)pyrimidin-4-yl]carbonyl}amino)benzoic acid

5-bromo-2-({[5-chloro-2-(methylthio)pyrimidin-4-yl]carbonyl}amino)benzoic acid

5-bromo-2-{[(5-chloro-2-morpholin-4-ylpyrimidin-4-yl)carbonyl]amino}benzoic acid

2-{[(1-benzyl-5-methoxy-1H-pyrazol-3-yl)carbonyl]amino}-5-bromobenzoic acid

2-({[5-(benzyloxy)-1-methyl-1H-pyrazol-3-yl]carbonyl}amino)-5-bromobenzoic acid

2-[({2-[4-(acetyloxy)phenyl]-1,3-dioxo-2,3-dihydro-1H-isoindol-5yl}carbonyl)amino]-5-bromobenzoic acid

2-({[5-({[2-(4-aminophenyl)ethyl]amino}sulfonyl)thien-3-yl]carbonyl}anino)-5-bromobenzoic acid

5-bromo-2-[({⁵-[(2,3-dihydro-1H-inden-2-ylamino)sulfonyl]thien-3yl}carbonyl)amino]benzoic acid

2-{[(5-{[(1-benzylpiperidin-4-yl)aamino]sulfonyl}thien-3-yl)carbonyl]aamino}-acid

5-bromo-2-{[(5-{[4-(4-chlorophenyl)piperazin-1-yl]sulfonyl}thien-3yl)carbonyl]amino}benzoic acid

5-bromo-2-({[5-({[4-(dimethylamino)benzyl]amino}sulfonyl)thien-3yl]carbonyl}amino)benzoic acid

5-bromo-2-({[4-({[4-(dimethylamino)benzyl]amino}sulfonyl)-5-methylthien-2yl]carbonyl}amino)benzoic acid

5-bromo-2-{[(5-{[(4-chlorophenyl)(methyl)aamino]sulfonyl}thien-3yl)carbonyl]amino}benzoic acid

5-bromo-2-({[5-(2,3-dihydro-1H-indol-1-ylsulfonyl)thien-3yl]carbonyl}amino)benzoic acid

5-bromo-2-[({5-[(2,3-dihydro-1,4-benzodioxin-6-ylamino)sulfonyl]thien-3yl}carbonyl)amino]benzoic acid

5-bromo-2-[({4-[(2,3-dihydro-1,4-benzodioxin-6-ylamino)sulfonyl]-3-methylthien-2yl}carbonyl)amino]benzoic acid

5-bromo-2-{[(4-{[(3-chloro-4-fluorophenyl)amino]sulfonyl}-5-methylthien-2yl)carbonyl]amino}benzoic acid

5-bromo-2-{[(3-methyl-4-{[(4-morpholin-4-ylphenyl)amino]sulfonyl}thien-2yl)carbonyl]amino}benzoic acid

5-bromo-2-({[4-({[4-(diethylamino)phenyl]amino}sulfonyl)-3-methylthien-2yl]carbonyl}amino)benzoic acid

5-bromo-2-[({5-[(6-chloro-2,3-dihydro-1H-indol-1-yl)sulfonyl]thien-3yl}carbonyl)amino]benzoic acid

5-bromo-2-[({4-[(6-chloro-2,3-dihydro-1H-indol-1-yl)sulfonyl]-5-methylthien-2yl}carbonyl)amino]benzoic acid

2-[({5-[(5-benzyl-2,5-diazabicyclo[2.2 .1 ]hept-2-yl)sulfonyl]thien-3yl}carbonyl)amino]-5-bromobenzoic acid

2-[({4-[(5-benzyl-2,5-diazabicyclo[2.2. 1 ]hept-2-yl)sulfonyl]-3-methylthien-2yl}carbonyl)amino]-5-bromobenzoic acid

2-[({4-[(5-benzyl-2,5-diazabicyclo[2.2.1 ]hept-2-yl)sulfonyl]-5-methylthien-2yl}carbonyl)anino]-5-bromobenzoic acid

5-bromo-2-(2-fuiroylamino)benzoic acid

5-bromo-2-[(5-bromo-2-furoyl)amino]benzoic acid

5-bromo-2-[(1H-pyrrol-2-ylcarbonyl)amino]benzoic acid

5-bromo-2-(3-furoylamino)benzoic acid

5-bromo-2-{[(3-methylthien-2-yl)carbonyl]amino}benzoic acid

5-bromo-2-{[(5-methylthien-2-yl)carbonyl]amino}benzoic acid

5-bromo-2-[(thien-3-ylcarbonyl)amino]benzoic acid

5-bromo-2-[(1H-indol-3-ylcarbonyl)amino]benzoic acid

2-[(1,3-benzodioxol-5-ylcarbonyl)amino]-5-bromobenzoic acid

2-[(1-benzofuran-2-ylcarbonyl)amino]-5-bromobenzoic acid

5-bromo-2-{[(2-chloropyridin-3-yl)carbonyl]amino}benzoic acid

5-bromo-2-[(pyridin-2-ylcarbonyl)amino]benzoic acid

5-bromo-2-{[(5-butylpyridin-2-yl)carbonyl]amino}benzoic acid

5-bromo-2-{[(2-phenylquinolin-4-yl)carbonyl]amino}benzoic acid

5-bromo-2-[(quinolin-2-ylcarbonyl)amino]benzoic acid

5-bromo-2-[(quinolin-4-ylcarbonyl)amino]benzoic acid

5-bromo-2-({[2-(methylthio)pyridin-3-yl]carbonyl}amino)benzoic acid

5-bromo-2-[(quinoxalin-2-ylcarbonyl)amino]benzoic acid

5-bromo-2-{[(3-methyl-i H-inden-2-yl)carbonyl]amino}benzoic acid

5-bromo-2-{[(7-methoxy-1-benzofuiran-2-yl)carbonyl]amino}benzoic acid

5-bromo-2-{[(5-methylpyrazin-2-yl)carbonyl]amino}benzoic acid

5-bromo-2-{[(5,6-dichloropyridin-3-yl)carbonyl]amino}benzoic acid

5-bromo-2-{[(10,10-dioxido-9-oxo-9H-thioxanthen-3-yl)carbonyl]amino}benzoic acid

5-bromo-2-{[(2,6-dimethoxypyridin-3-yl)carbonyl]amino}benzoic acid

5-bromo-2-{[(5-bromothien-2-yl)carbonyl]amino}benzoic acid

5-bromo-2-[(2,5-dimethyl-3-furoyl)amino]benzoic acid

5-bromo-2-{[(6-bromopyridin-2-yl)carbonyl]amino}benzoic acid

5-bromo-2-{[5-(4-nitrophenyl)-2-furoyl]amino}benzoic acid

5-bromo-2-{[5-(4-chlorophenyl)-2-furoyl]amino}benzoic acid

5-bromo-2-[(2,6-dichloroisonicotinoyl)amino]benzoic acid

5-bromo-2-({5-[3-(trifluoromethyl)phenyl]-2-furoyl}amino)benzoic acid

5-bromo-2-{[(4-{[(3-chloro-4-fluorophenyl)amino]sulfonyl}-3-methylthien-2yl)carbonyl]amino}benzoic acid

5-bromo-2-{[(5-methyl-4-{[(2-methyl-1,3-benzothiazol-5-yl)amino]sulfonyl}thien-2-yl)carbonyl]amino}benzoic acid

5-bromo-2-[({4-[({4-[(diethoxyphosphoryl)methyl]phenyl}amino)sulfonyl]-5methylthien-2-yl}carbonyl)amino]benzoic acid

5-bromo-2-{[(4-{[(4-chlorophenyl)amino]sulfonyl}-5-methylthien-2-yl)carbonyl]amino}benzoic acid

5-bromo-2-({[5-({[4-(diethylamino)phenyl]amino}sulfonyl)thien-3yl]carbonyl}amino)benzoic acid

5-bromo-2-[({4-[(2,3-dihydro-1H-inden-2-ylamino)sulfonyl]-3-methylthien-2-yl}carbonyl)amino]benzoic acid

5-bromo-2-[({4-[(2,3-dihydro-1,4-benzodioxin-6-ylamino)sulfonyl]-5-methylthien-2yl}carbonyl)amino]benzoic acid

2-({[4-({[2-(4-aminophenyl)ethyl]amino}sulfonyl)-5-methylthien-2yl]carbonyl}amino)-5-bromobenzoic acid

5-bromo-2-{[(4-{[4-(4-chlorophenyl)piperazin-1-yl]sulfonyl}-5-methylthien-2-yl)carbonyl]amino}benzoic acid

5-bromo-2-[({4-[(2,3-dihydro-1H-inden-2-ylamino)sulfonyl]-5-methylthien-2-yl}carbonyl)amino]benzoic acid

2-{[(3-benzoylpyridin-2-yl)carbonyl]amino}-5-bromobenzoic acid

5-bromo-2-{[(2-isobutyl-1,3-dioxo-2,3-dihydro-1H-isoindol-5-yl)carbonyl]amino}benzoic acid

5-bromo-2-({[1,3-dioxo-2-(tetrahydrofuran-2-ylmethyl)-2,3-dihydro-1H-isoindol-5-yl]carbonyl}acid

2-({[6-(benzylthio)pyridin-3-yl]carbonyl}amino)-5-cyanobenzoic acid hydrochloride

5-bromo-2-({[2-(methylsulfonyl)-5-morpholin-4-ylpyrimidin-4-yl]carbonyl}amino)benzoic acid

2-({[2-(4-chlorophenyl)-1,1-dioxido-3,4-dihydro-2H-1,2-benzothiazin-7yl]carbonyl}acid

5-cyano-2-({[6-(methylthio)pyridin-3-yl]carbonyl}amino)benzoic acid hydrochloride

5-cyano-2-({[6-(ethylthio)pyridin-3-yl]carbonyl}amino)benzoic acid hydrochloride

2-{[(6-chloropyridin-3-yl)carbonyl]amino}-5-cyanobenzoic acid

5-cyano-2-{[(3-methylthien-2-yl)carbonyl]amino}benzoic acid

5-cyano-2-(3-fiiroylamino)benzoic acid

5-cyano-2-{[(3-methyl-1H-inden-2-yl)carbonyl]amino}benzoic acid

2-{[(6-bromopyridin-2-yl)carbonyl]amino}-5-cyanobenzoic acid

5-cyano-2-[(2,5-dimethyl-3-furoyl)amino]benzoic acid

5-cyano-2-[(thien-3-ylcarbonyl)amino]benzoic acid

5-cyano-2-[(pyridin-2-ylcarbonyl)amino]benzoic acid

5-cyano-2-{[(2,6-dimethoxypyridin-3-yl)carbonyl]amino}benzoic acid

2-[(1,3-benzodioxol-5-ylcarbonyl)amino]-5-cyanobenzoic acid

5-cyano-2-[(quinolin-2-ylcarbonyl)amino]benzoic acid

2-{[(5-bromopyridin-3-yl)carbonyl]amino}-5-cyanobenzoic acid

5-cyano-2-{[(6-methoxypyridin-3-yl)carbonyl]amino}benzoic acid

5-cyano-2-({[5-(1-methyl-1H-pyrrol-2-yl)pyridin-3-yl]carbonyl}amino)benzoic acid

5-cyano-2-({[5-(4-methoxyphenyl)pyridin-3-yl]carbonyl}amino)benzoic acid

5-cyano-2-{[(5-pyrazin-2-ylpyridin-3-yl)carbonyl]amino}benzoic acid

2-[(2,3′-bipyridin-5′-ylcarbonyl)amino]-5-cyanobenzoic acid

5-cyano-2-({[5-(2-fiiryl)pyridin-3-yl]carbonyl}amino)benzoic acid

5-cyano-2-({[5-(2-methylphenyl)pyridin-3-yl]carbonyl}amino)benzoic acid

5-cyano-2-[({5-[2-(trifluoromethyl)phenyl]pyridin-3-yl}carbonyl)amino]benzoic acid

2-({[5-(4-tert-butylphenyl)pyridin-3-yl]carbonyl}amino)-5-cyanobenzoic acid

2-({[5-(4-chlorophenyl)pyridin-3-yl]carbonyl}amino)-5-cyanobenzoic acid

5-cyano-2-({[6-(propylthio)pyridin-3-yl]carbonyl}amino)benzoic acid hydrochloride

5-cyano-2-[(1H-indol-2-ylcarbonyl)aamino]benzoic acid

2-[(1-benzofiuran-2-ylcarbonyl)amino]-5-cyanobenzoic acid

5-cyano-2-[(pyrazin-2-ylcarbonyl)amino]benzoic acid

5-cyano-2-{[(5-phenoxypyridin-3-yl)carbonyl]amino}benzoic acid

2-({[6-(butylthio)pyridin-3-yl]carbonyl}amino)-5-cyanobenzoic acid

2-[({6-[(5-chloro-2,3-dihydro-1H-indol-1-yl)sulfonyl]pyridin-3-yl}carbonyl)amino]-5cyanobenzoic acid

5-cyano-2-({[5-(3-hydroxyprop-1-ynyl)pyridin-3-yl]carbonyl}amino)benzoic acid

5-cyano-2-[({6-[(1-phenylethyl)thio]pyridin-3-yl}carbonyl)amino]benzoic acid

5-cyano-2-({[⁶-(isopropylthio)pyridin-3-yl]carbonyl}amino)benzoic acid

5-cyano-2-({[6-(cyclopentylthio)pyridin-3-yl]carbonyl}amino)benzoic acid

5-cyano-2-{[(2-pyridin-3-yl-1,3-thiazol-4-yl)carbonyl]amino}benzoic acid

2-({[2-(benzylthio)-4-ethoxypyrimidin-5-yl]carbonyl}amino)-5-cyanobenzoic acid

2-({[5-(benzylthio)-1,3,4-thiadiazol-2-yl]carbonyl}amino)-5-cyanobenzoic acid

5-cyano-2-{[(2-phenylquinolin-4-yl)carbonyl]amino}benzoic acid

5-cyano-2-(2-furoylamino)benzoic acid

5-cyano-2-[({4-[(2,6-dimethylmorpholin-4-yl)sulfonyl]-5-methylthien-2yl}carbonyl)amino]benzoic acid

5-cyano-2-{[(4-{[cyclohexyl(methyl)amino]sulfonyl}-5-methylthien-2yl)carbonyl]amino}benzoic acid

2-{[(4-{[4-(4-acetylphenyl)piperazin-1-yl]sulfonyl}-5-methylthien-2yl)carbonyl]amino}-5-cyanobenzoic acid

5-cyano-2-({[4-({3-[(diethylamino)carbonyl]piperidin-1-yl}sulfonyl)-5-methylthien-2yl]carbonyl}amino)benzoic acid

2-{[(4-{[allyl(cyclopentyl)amino]sulfonyl}-5-methylthien-2-yl)carbonyl]amino}-5cyanobenzoic acid

5-cyano-2-[({4-[(diisobutylamino)sulfonyl]-5-methylthien-2yl}carbonyl)amino]benzoic acid

5-cyano-2-[({5-methyl-4-[(4-pyrimidin-2-ylpiperazin-1-yl)sulfonyl]thien-2yl}carbonyl)amino]benzoic acid

2-{[(4-{[benzyl(2-cyanoethyl)amino]sulfonyl}-5-methylthien-2-yl)carbonyl]amino}acid

2-{[(4-{[benzyl(2-hydroxyethyl)amino]sulfonyl}-5-methylthien-2-yl)carbonyl]anino}5-cyanobenzoic acid

2-({[4-(azepan-1-ylsulfonyl)-5-methylthien-2-yl]carbonyl}anino)-5-cyanobenzoic acid

5-cyano-2-({[5-methyl-4-({4-[3-(trifluoromethyl)phenyl]piperazin-1-yl}sulfonyl)thien-2-yl]carbonyl}amino)benzoic acid

5-cyano-2-{[(5-methyl-4-{[2-(pyrrolidin-1-ylmethyl)pyrrolidin-1-yl]sulfonyl}thien-2-yl)carbonyl]amino}benzoic acid

5-cyano-2-({[5-methyl-4-(morpholin-4-ylsulfonyl)thien-2-yl]carbonyl}amino)benzoic acid

5-cyano-2-{[(5-methyl-4-{[4-(4-nitrophenyl)piperazin-1-yl]sulfonyl}thien-2-yl)carbonyl]amino}benzoic acid

2-{[(4-{[butyl(ethyl)amino]sulfonyl}-5-methylthien-2-yl)carbonyl]amino}-5-cyanobenzoic acid

5-cyano-2-{[(4-{[(2-hydroxy-2-phenylethyl)(methyl)aamino]sulfonyl}-5-methylthien-2-yl)carbonyl]amino}benzoic acid

5-cyano-2-{[(4-{[2-(methoxymethyl)pyrrolidin-1-yl]sulfonyl}-5-methylthien-2-yl)carbonyl]amino}benzoic acid

5-cyano-2-[({4-[(4-cyano-4-phenylpiperidin-1-yl)sulfonyl]-5-methylthien-2-yl}carbonyl)amino]benzoic acid

5-cyano-2-[({4-[(3,5-dimethylpiperidin-1-yl)sulfonyl]-5-methylthien-2-yl}carbonyl)amino]benzoic acid

5-cyano-2-{[(4-{[[2-(diethylamino)ethyl](ethyl)amino]sulfonyl}-5-methylthien-2-yl}carbonyl)amino]benzoic acid

2-{[(4-{[benzyl(isopropyl)amino]sulfonyl}-5-methylthien-2-yl)carbonyl]amino}-5-cyanobenzoic acid

5-cyano-2-[({5-methyl-4-[(4-phenylpiperazin-1-yl)sulfonyl]thien-2-yl}carbonyl)amino]benzoic acid

5-cyano-2-[({5-methyl-4-[(2-methylpyrrolidin-1-yl)sulfonyl]thien-2-yl}carbonyl)amino]benzoic acid

5-cyano-2-{[(4-{[4-(4-fluorophenyl)piperazin-1-yl]sulfonyl}-5-methylthien-2-yl)carbonyl]amino}benzoic acid

5-cyano-2-[({5-methyl-4-[(4-pyridin-2-ylpiperazin-1-yl)sulfonyl]thien-2-yl}carbonyl)amino]benzoic acid

5-cyano-2-[({5-methyl-4-[(4-methylpiperidin-1-yl)sulfonyl]thien-2-yl}carbonyl)amino]benzoic acid

5-cyano-2-[({4-[(2,3-dihydro-1H-inden-5-ylamino)sulfonyl]-5-methylthien-2-yl}carbonyl)amino]benzoic acid

5-cyano-2-{[(5-methyl-4-{[methyl(2-phenylethyl)amino]sulfonyl}thien-2-yl)carbonyl]amino}benzoic acid

2-({[4-(azocan-1-ylsulfonyl)-5-methylthien-2-yl]carbonyl}amino)-5-cyanobenzoic acid 5-cyano-2-{[(4-{[(2-hydroxy-1-methyl-2-phenylethyl)(methyl)amino]sulfonyl}-5-methylthine-2-yl)carbonyl]amino}benzoic acid

5-cyano-2-[({5-methyl-4-[(3-methylpiperidin-1-yl)sulfonyl]thien-2-yl}carbonyl)amino]benzoic acid

5-cyano-2-{[(4-{[(3-hydroxy-3-phenylpropyl)(methyl)amino]sulfonyl}-5-methylthien-2-yl)carbonyl]amino}benzoic acid

2-{[(4-{[butyl(cyanomethyl)amino]sulfonyl}-5-methylthien-2-yl)carbonyl]amino}-5-cyanobenzoic acid

5-cyano-2-({[5-methyl-4-(pyrrolidin-1-ylsulfonyl)thien-2-yl]carbonyl}anino)benzoic acid

2-[({4-[(4-benzylpiperazin-1-yl)sulfonyl]-5-methylthien-2-yl}carbonyl)amino]-5-cyanobenzoic acid

5-cyano-2-{[(4-{[(2-cyanoethyl)(methyl)amino]sulfonyl}-5-methylthien-2-yl}carbonyl]amino-5-cyanobenzoic acid

5-cyano-2-{[(4-{[(2-hydroxy-1-methyl-2-phenylethyl)(methyl)amino]sulfonyl}-5methylthien-2-yl)carbonyl]amino}benzoic acid

5-cyano-2-[({4-[(4-hydroxypiperidin-1-yl)sulfonyl]-5-methylthien-2-yl)carbonyl]amino}benzoic benzoic acid

5-cyano-2-({[5-methyl-4-(octahydroquinolin-1 (2H)-ylsulfonyl)thien-2-yl]carbonyl}amino)benzoic acid

5-cyano-2-{[(5-methoxy-1H-indol-2-yl)carbonyl]amino}benzoic acid

2-({[5-(benzyloxy)-1H-indol-2-yl]carbonyl}amino)-5-cyanobenzoic acid

5-cyano-2-({[5-(3,5-dimethylisoxazol-4-yl)pyridin-3-yl]carbonyl}amino)benzoic acid

5-cyano-2-{[(5-quinolin-3-ylpyridin-3-yl)carbonyl]amino}benzoic acid

5-cyano-2-[({5-[4-(trifluoromethyl)phenyl]pyridin-3-yl}carbonyl)amino]benzoic acid

5-cyano-2-[({5-[3-(trifluoromethyl)phenyl]pyridin-3-yl}carbonyl)amino]benzoic acid

5-cyano-2-({[5-(2,4-dimethoxypyrimidin-5-yl)pyridin-3-yl]carbonyl}amino)benzoic acid

5-cyano-2-{[(5-phenylpyridin-3-yl)carbonyl]amino}benzoic acid

2-({[2-(benzylthio)-1,3-thiazol-5-ylJcarbonyl}amino)-5-cyanobenzoic acid

5-isocyano-2-{[(5-phenyl-1,2,4-oxadiazol-3-yl)carbonyl]amino}benzoic acid

5-isocyano-2-{[(2-phenyl-1,3-oxazol-4-yl)carbonyl]amino}benzoic acid

2-{[(2-tert-butyl-1,3-dioxo-2,3-dihydro-1H-isoindol-5-yl)carbonyl]amino}-5-cyanobenzoic acid

5-bromo-2-{[(2-tert-butyl-1,3-dioxo-2,3-dihydro-1H-isoindol-5-yl)carbonyl]amino}benzo acid

5-cyano-2-{[(4-{[2-(hydroxymethyl)pyrrolidin-1-yl]sulfonyl}-5-methylthien-2-yl)carbonyl]amino}benzoic acid

2-{[(4-{[4-(4-bromophenyl)-4-hydroxypiperidin-1-yl]sulfonyl}-5-methylthien-2-yl)carbonyl]amino}-5-cyanobenzoic acid

2-{[(4-{[butyl(2-hydroxyethyl)amino]sulfonyl}-5-methylthien-2-yl)carbonyl]amino}-5-cyanobenzoic acid

5-cyano-2-{[(5-quinolin-8-ylpyridin-3-yl)carbonyl]amino}benzoic acid

5-cyano-2-{[(6-morpholin-4-ylpyridin-3-yl)carbonyl]amino}benzoic acid

5-cyano-2-({[5-(1H-pyrazol-4-ylethynyl)pyridin-3-yl]carbonyl}amino)benzoic acid

5-cyano-2-[({5-[(2,4-dimethoxypyrimidin-5-yl)ethynyl]pyridin-3-yl}carbonyl)amino]benzoic acid

5-cyano-2-{[(5-ethynylpyridin-3-yl)carbonyl]amino}benzoic acid

5-cyano-2-[({4-[(2-ethylpiperidin-1-yl)sulfonyl]-5-methylthien-2-yl}carbonyl)amino]benzoic acid

5-cyano-2-[({4-[(6,7-dimethoxy-3,4-dihydroisoquinolin-2(1H)-yl)sulfonyl]-5methylthien-2-yl}carbonyl)amino]benzoic acid

5-cyano-2-({[4-(3,4-dihydroisoquinolin-2(1H)-ylsulfonyl)-5-methylthien-2-yl]carbonyl}amino)benzoic acid

2-({[6-(benzylsulfonyl)pyiidin-3-yl]carbonyl}amino)-5-cyanobenzoic acid

5-cyano-2-{[(6-piperidin-1-ylpyridin-3-yl)carbonyl]amino}benzoic acid

5-cyano-2-[({5-[(3,5-dirnethylisoxazol-4-yl)ethynyl]pyridin-3-yl}carbonyl)amino]benzoic acid

5-cyano-2-({[5-(phenylethynyl)pyridin-3-yl]carbonyl}amino)benzoic acid

5-cyano-2-[({5-[2-(1,5-dimnethyl-3-oxo-2-phenyl-2,3-dihydro-1H-pyrazol-4-yl)-2-oxoethyl]pyridin-3-yl}carbonyl)amino]benzoic acid

5-cyano-2-{[(5-phenylisoxazol-3-yl)carbonyl]amino}benzoic acid

5-cyano-2-({[6-(tetrahydro-2H-pyran-4-ylthio)pyridin-3-yl]carbonyl}amino)benzoic acid

5-cyano-2-({[5-(ethylthio)pyridin-3-yl]carbonyl}amino)benzoic acid

2-({[2-(benzylthio)-1,3-thiazol-4-yl]carbonyl}amino)-5-cyanobenzoic acid

5-bromo-2-{[(5-phenylisoxazol-3-yl)carbonyl]amino}benzoic acid 2-{[(6-{[3-(4-bromophenoxy)propyl]thio}pyridin-3-yl)carbonyl]amino}-5-cyanobenzoic acid

5-cyano-2-[({6-[(cyclopropylmethyl)thio]pyridin-3-yl}carbonyl)amino]benzoic acid

5-bromo-2-({[6-(isopropylthio)pyridin-3-yl]carbonyl}amino)benzoic acid

2-({[5-(benzylthio)pyrazin-2-yl]carbonyl}amino)-5-cyanobenzoic acid

5-cyano-2-{[(5-methoxy-1-benzofuran-2-yl)carbonyl]amino}benzoic acid

5-cyano-2-{[(7-methoxy-1-benzofuran-2-yl)carbonyl]amino}benzoic acid

5-cyano-2-{[(1-methyl-1H-indol-2-yl)carbonyl]amino}benzoic acid

5-cyano-2-({[6-(cyclohexylthio)pyridin-3-yl]carbonyl}amino)benzoic acid

2-({[5-(benzylthio)isoxazol-3-yl]carbonyl}anino)-5-bromobenzoic acid

5-bromo-2-({[6-(cyclohexylthio)pyridin-3-yl]carbonyl}amino)benzoic acid

2-({[5-(benzylthio)isoxazol-3-yl]carbonyl}amino)-5-cyanobenzoic acid

2-({[5-(benzylthio)pyrazin-2-yl]carbonyl}amino)-5-bromobenzoic acid

2-({[6-(sec-butylthio)pyridin-3-yl]carbonyl}anino)-5-cyanobenzoic acid

5-cyano-2-({[6-(pentylthio)pyridin-3-yl]carbonyl}amino)benzoic acid

2-({[5-(benzylthio)-1,3,4-oxadiazol-2-yl]carbonyl}amino)-5-bromobenzoic acid

2-({[6-(benzyloxy)-1H-indol-2-yl]carbonyl}aiino)-5-cyanobenzoic acid

N-[4-bromo-2-(5-oxo-4,5-dihydro-1,2,4-oxadiazol-3-yl)phenyl]-5-phenylisoxazole-3carboxamide

2-{[(7-chloro-1H-indol-2-yl)carbonyl]amino}-5-cyanobenzoic acid

5-cyano-2-{[(4-methoxy-1H-indol-2-yl)carbonyl]amino}benzoic acid

5-bromo-2-{[(1-methyl-1H-indol-2-yl)carbonyl]amino}benzoic acid

2-{[(6-chloro-1H-indol-2-yl)carbonyl]amino}-5-cyanobenzoic acid

2-{[(1-benzyl-1H-indol-2-yl)carbonyl]anino}-5-cyanobenzoic acid

5-cyano-2-{[(1-ethyl-1H-indol-2-yl)carbonyl]amino}benzoic acid

5-cyano-2-({[7-(phenylsulfonyl)-1H-indol-2-yl]carbonyl}anino)benzoic acid

5-cyano-2-{[(5-phenyl-1,3-oxazol-2-yl)carbonyl]amino}benzoic acid

2-({[5-(benzylthio)-1,3,4-thiadiazol-2-yl]carbonyl}amino)-5-bromobenzoic acid

5-cyano-2-{[(2-phenylfuro[2,3-c]pyridin-5-yl)carbonyl]amino}benzoic acid

5-cyano-2-({[6-(hexylthio)pyridin-3-yl]carbonyl}amino)benzoic acid

5-cyano-2-[({6-[(3-ethoxy-3-oxopropyl)thio]pyridin-3-yl}carbonyl)amino]benzoic acid

2-({[6-(benzylthio)pyrazin-2-yl]carbonyl}anino)-5-cyanobenzoic acid

2-({[6-(benzylthio)pyrazin-2-yl]carbonyl}amino)-5-bromobenzoic acid

2-({[5-(3-chlorophenyl)-1,3-thiazol-2-yl]carbonyl}amino)-5-cyanobenzoic acid

5-cyano-2-{[(5-phenyl-1,3-thiazol-2-yl)carbonyl]amino}benzoic acid

5-cyano-2-{[(4-phenyl-1,3-thiazol-2-yl)carbonyl]amino}benzoic acid

2-[(1,3-benzothiazol-2-ylcarbonyl)amino]-5-cyanobenzoic acid

5-cyano-2-[(thieno[2,3-b]pyridin-2-ylcarbonyl)amino]benzoic acid

5-cyano-2-{[(3-methylfuro[2,3-c]pyridin-5-yl)carbonyl]amino}benzoic acid

5-bromo-2-({[5-(pentylthio)-1,3,4-oxadiazol-2-yl]carbonyl}amfino)benzoic acid

5-cyano-2-{[(6-{[3-(2-methoxyethoxy)propyl]thio}pyridin-3-yl)carbonyl]amino}benzoic acid

5-chloro-2-{[(5-phenyl-1,3-oxazol-2-yl)carbonyl]amino}benzoic acid

5-bromo-2-({[6-(tetrahydro-2H-pyran-4-ylthio)pyridin-3-yl]carbonyl}amino)benzoic acid

5-cyano-2-({[6-(nonylthio)pyridin-3-yl]carbonyl}amino)benzoic acid

5-cyano-2-{[(6-{[(3,5-dimethylisoxazol-4-yl)methyl]thio}pyridin-3-yl)carbonyl]amino}benzoic acid

5-cyano-2-{[(5-methylisoxazol-3-yl)carbonyl]amino}benzoic acid

5-cyano-2-[({6-[(2-methoxy-2-oxoethyl)thio]pyridin-3-yl}carbonyl)armino]benzoic acid

5-bromo-2-({[6-(hexylthio)pyridin-3-yl]carbonyl}amino)benzoic acid

5-chloro-2-({[6-(hexylthio)pyridin-3-yl]carbonyl}amino)benzoic acid

5-bromo-2-{[(6-{[3-(2-methoxyethoxy)propyl]thio}pyridin-3-yl)carbonyl]amino}benzoic acid

2-{[(1-allyl-1H-indol-2-yl)carbonyl]amino}-5-cyanobenzoic acid

5-cyano-2-({[1-(cyclohexylmethyl)-1H-indol-2-yl]carbonyl}amino)benzoic acid

5-cyano-2-({[1-(2-methoxyethyl) -1H-indol-2-yl]carbonyl}amino)benzoic acid

5-cyano-2-{[(1-pentyl-1H-indol-2-yl)carbonyl]amino}benzoic acid

2-{[(1-butyl-1H-indol-2-yl)carbonyl]amino}-5-cyanobenzoic acid

5-cyano-2-{[(1-propyl-1H-indol-2-yl)carbonyl]amiino}benzoic acid

5-chloro-2-{[(1-propyl-1H-indol-2-yl)carbonyl]amrino}benzoic acid

2-{[(1-butyl-1H-indol-2-yl)carbonyl]amino}-5-chlorobenzoic acid

5-chloro-2-{[(1-pentyl-1H-indol-2-yl) carbonyl] amino }benzoic acid

5-chloro-2-({[1-(2-methoxyethyl)-1H-indol-2-yl]carbonyl}amino)benzoic acid

5-chloro-2-({[1-(cyclohexylmethyl)-1H-indol-2-yl]carbonyl}amino)benzoic acid

2-{[(1-allyl-1H-indol-2-yl)carbonyl]amino}-5-chlorobenzoic acid

2-{[(1-allyl-1H-indol-2-yl)carbonyl]amino}-5-bromobenzoic acid

5-bromo-2-({[1-(cyclohexylmethyl)-1H-indol-2-yl]carbonyl}amino)benzoic acid

5-bromo-2-({[1-(2-methoxyethyl) -1H-indol-2-yl]carbonyl}amino)benzoic acid

5-bromo-2-{[(1-pentyl-1H-indol-2-yl)carbonyl]amino}benzoic acid

5-bromo-2-{[(1-butyl-1H-indol-2-yl)carbonyl]amino}benzoic acid

5-bromo-2-{[(1-propyl-1H-indol-2-yl)carbonyl]amino}benzoic acid

2-({[4-(benzyloxy)pyridin-2-yl]carbonyl}amino)-5-bromobenzoic acid

2-{[(1-benzyl-1H-indol-2-yl)carbonyl]amino}-5-chlorobenzoic acid

2-{[(1-benzyl-1H-indol-2-yl)carbonyl]anino}-5-bromobenzoic acid

5-bromo-2-{[(1-isopropyl-1H-indol-2-yl)carbonyl]amino}benzoic acid

5-cyano-2-{[(1-isopropyl-1H-indol-2-yl)carbonyl]amino}benzoic acid

5-chloro-2-{[(1-methyl-1H-indol-2-yl)carbonyl]amino}benzoic acid

5-chloro-2-{[(1-isobutyl-1H-indol-2-yl)carbonyl]amino}benzoic acid

5-bromo-2-{[(1-isobutyl-1H-indol-2-yl)carbonyl]amino}benzoic acid

5-cyano-2-{[(1-isobutyl-1H-indol-2-yl)carbonyl]amino}benzoic acid

5-cyano-2-({[1-(3-phenylpropyl)-1H-indol-2-yl]carbonyl}amino)benzoic acid

5-chloro-2-({[1-(3-phenylpropyl)-1H-indol-2-yl]carbonyl}amino)benzoic acid

5-bromo-2-({[1-(3-phenylpropyl)-1H-indol-2-yl]carbonyl}amino)benzoic acid

5-chloro-2-{[(6-{[3-(2-methoxyethoxy)propyl]thio}pyridin-3-yl)carbonyl]amino}benzoic acid

5-cyano-2-{[(4,5-dichloroisothiazol-3-yl)carbonyl]amino}benzoic acid

2-{[(4-chloro-5-phenylisothiazol-3-yl)carbonyl]amino}-5-cyanobenzoic acid

5-bromo-2-{[(4-chloro-1-oxidopyridin-2-yl)carbonyl]amino}benzoic acid

5-cyano-2-{[(3-phenylisoxazol-5-yl)carbonyl]amino}benzoic acid

5-chloro-2-[(E)-2-(5-phenylisoxazol-3-yl)ethenyl]benzoic acid

5-cyano-2-[(E)-2-(5-phenylisoxazol-3-yl)ethenyl]benzoic acid

2-({[4-(benzyloxy)pyridin-2-yl]carbonyl}amino)-5-cyanobenzoic acid

2-{[(6-{[(5-chloro-1,2,4-thiadiazol-3-yl)methyl]thio}pyridin-3-yl)carbonyl]amino}-5-cyanobenzoic acid

5-cyano-2-[({6-[(1,2,4-oxadiazol-3-ylmethyl)thio]pyridin-3-yl}carbonyl)amino]benzoic acid

2-({[4-(benzyloxy)-1-oxidopyridin-2-yl]carbonyl}amino)-5-bromobenzoic acid

2-({[4-(benzylthio)-1-oxidopyridin-2-yl]carbonyl}amino)-5-bromobenzoic acid

5-cyano-2-({[5-(4-methoxyphenyl)isoxazol-3-yl]carbonyl}amino)benzoic acid

5-cyano-2-[({5-[4-(trifluoromethyl)phenyl]isoxazol-3-yl}carbonyl)amino]benzoic acid

2-({[5-(2-chlorophenyl)isoxazol-3-yl]carbonyl}anino)-5-cyanobenzoic acid

5-cyano-2-({[5-(2-fluorophenyl)isoxazol-3-yl]carbonyl}amino)benzoic acid

2-{[(6-{[4-(acetyloxy)butyl]thio}pyridin-3-yl)carbonyl]amino}-5-cyanobenzoic acid

5-cyano-2-{[(5-methyl-2-phenyl-2H-1,2,3-triazol-4-yl)carbonyl]amino}benzoic acid

5-cyano-2-[({6-[(4-hydroxybutyl)thio]pyridin-3-yl}carbonyl)amino]benzoic acid

5-chloro-2-({[7-(phenylsulfonyl) -1H-indol-2-yl]carbonyl}amino)benzoic acid

5-bromo-2-({[7-(phenylsulfonyl)-1H-indol-2-yl]carbonyl}amino)benzoic acid

5-cyano-2-({[1-methyl-7-(phenylsulfonyl)-1H-indol-2-yl]carbonyl)amino}benzoic acid

5-cyano-2-({[5-(4-methoxyphenyl)-1,3-oxazol-2-yl]carbonyl)amino}benzoic acid

5-cyano-2-({[5-(2-methoxyphenyl)-1,3-oxazol-2-yl]carbonyl)amino}benzoic acid

5-bromo-2-({[5-(2-methoxyphenyl)-1,3-oxazol-2-yl]carbonyl)amino}benzoic acid

5-chloro-2-({[5-(4-methoxyphenyl)isoxazol-3-yl]carbonyl)amino}benzoic acid

5-cyano-2-[({5-[2-(trifluoromethyl)phenyl]isoxazol-3-yl}carbonyl)amino]benzoic acid

2-{[(5-tert-butylisoxazol-3-yl)carbonyl]amino}-5-cyanobenzoic acid

5-cyano-2-{[(3,5-dimethyl-4,5′-biisoxazol-3′-yl)carbonyl]amino}benzoic acid

5-cyano-2-{[(4-methyl-5-phenylisoxazol-3-yl)carbonyl]amino}benzoic acid

5-bromo-2-[({5-[2-(trifluoromethyl)phenyl]isoxazol-3-yl}carbonyl)amino]benzoic acid

5-chloro-2-{[(4-methyl-5-phenylisoxazol-3-yl)carbonyl]amino}benzoic acid

5-chloro-2-[({5-[2-(trifluoromethyl)phenyl]isoxazol-3-yl}carbonyl)amino]benzoic acid

5-bromo-2-{[(4-methyl-5-phenylisoxazol-3-yl)carbonyl]amino}benzoic acid

5-bromo-2-[({5-[4-(trifluoromethyl)phenyl]isoxazol-3-yl}carbonyl)amino]benzoic acid

5-bromo-2-({[5-(4-{[(5-methylisoxazol-3-yl)amino]sulfonyl}phenyl)isoxazol-3-yl]carbonyl)amino}benzoic acid

5-chloro-2-{[(5-phenylisoxazol-3-yl)carbonyl]amino}benzoic acid

5-chloro-2-[({5-[4-(trifluoromethyl)phenyl]isoxazol-3-yl}carbonyl)amino]benzoic acid

5-bromo-2-({[5-(4-methoxyphenyl)isoxazol-3-yl]carbonyl}amino)benzoic acid

5-cyano-2-({[5-(3-cyclohexylpropyl)isoxazol-3-yl]carbonyl}amino)benzoic acid

5-cyano-2-({[5-(3-phenylpropyl)isoxazol-3-yl]carbonyl}amino)benzoic acid

5-cyano-2-({[5-(2-methoxyphenyl)isoxazol-3-yl]carbonyl}amino)benzoic acid

5-bromo-2-{[(5-phenyl-1,3-oxazol-2-yl)carbonyl]amino}benzoic acid

5-bromo-2-({[5-(4-methoxyphenyl)-1,3-oxazol-2-yl]carbonyl}amino)benzoic acid

2-({[5-(1,3-benzodioxol-5-yl)-1,3-oxazol-2-yl]carbonyl}amino)-5-bromobenzoic acid

5-bromo-2-({[1-methyl-7-(phenylsulfonyl)-1H-indol-2-yl]carbonyl}amino)benzoic acid

5-chloro-2-({[1-methyl-7-(phenylsulfonyl)-1H-indol-2-yl]carbonyl}amino)benzoic acid

5-cyano-2-[(isoquinolin-3-ylcarbonyl)amino]benzoic acid

5-cyano-2-[({7-[(phenylacetyl)amino]-1H-indol-2-yl}carbonyl)amino]benzoic acid

2-({[7-(benzoylamino)-1H-indol-2-yl]carbonyl}anino)-5-cyanobenzoic acid

2-{[(7-{[(acetyloxy)acetyl]amino}-1H-indol-2-yl)carbonyl]amino}-5-cyanobenzoic acid

5-cyano-2-[({7-[(cyclopentylcarbonyl)amino]-1H-indol-2-yl}carbonyl)amino]benzoic acid

2-{[(7-amino-1H-indol-2-yl)carbonyl]amino}-5-cyanobenzoic acid

2-[(1,2-benzisoxazol-3-ylcarbonyl)amino]-5-cyanobenzoic acid

5-cyano-2-({[5-(4-fluorophenyl)-1,3-oxazol-2-yl]carbonyl}amino)benzoic acid

5-bromo-2-({[5-(3-cyclohexylpropyl)isoxazol-3-yl]carbonyl}amino)benzoic acid

5-bromo-2-({[5-(3-phenylpropyl)isoxazol-3-yl]carbonyl}amino)benzoic acid

5-bromo-2-({[5-(2-methoxyphenyl)isoxazol-3-yl]carbonyl}amino)benzoic acid

5-chloro-2-({[5-(3-cyclohexylpropyl)isoxazol-3-yl]carbonyl}anino)benzoic acid

5-chloro-2-({[5-(3-phenylpropyl)isoxazol-3-yl]carbonyl}anino)benzoic acid

5-chloro-2-({[5-(2-methoxyphenyl)isoxazol-3-yl]carbonyl}amino)benzoic acid

5-cyano-2-[({6-[(1-methylpentyl)thio]pyridin-3-yl}carbonyl)amino]benzoic acid

5-cyano-2-[({6-[(1-ethylpropyl)thio]pyridin-3-yl}carbonyl)amino]benzoic acid

5-bromo-2-({[5-(2-chlorophenyl)isoxazol-3-yl]carbonyl}amino)benzoic acid

5-bromo-2-({[5-(2-fluorophenyl)isoxazol-3-yl]carbonyl}amino)benzoic acid

5-bromo-2-({[5-(3-methoxyphenyl)-1,3-oxazol-2-yl]carbonyl}amino)benzoic acid

5-bromo-2-[({5-[2-(trifluoromethyl)phenyl]-1,3-oxazol-2-yl}carbonyl)amino]benzoic acid

2-{[(7-{[(6-chloropyridin-3-yl)carbonyl]amino}-1H-indol-2-yl)carbonyl]amino}acid

5-chloro-2-({[5-(2-fluorophenyl)isoxazol-3-yl]carbonyl}amino)benzoic acid

5-chloro-2-({[5-(2-chlorophenyl)isoxazol-3-yl]carbonyl}anino)benzoic acid

5-cyano-2-({[5-(2-methylphenyl)isoxazol-3-yl]carbonyl}amino)benzoic acid

5-cyano-2-({[5-(triisopropylsilyl)isoxazol-3-yl]carbonyl}amino)benzoic acid

5-cyano-2-[({7-[(isoxazol-5-ylcarbonyl)amino]-1H-indol-2-yl}carbonyl)amino]benzoic acid

5-cyano-2-[({7-[(2,4-difluorobenzoyl)amino]-1H-indol-2-yl}carbonyl)amino]benzoic acid

5-cyano-2-[({7-[(fluoroacetyl)amino]-1H-indol-2-yl}carbonyl)amino]benzoic acid

2-({[7-(acetylamino)-1H-indol-2-yl]carbonyl}anino)-5-cyanobenzoic acid

2-{[(7-{[(4-chlorophenyl)acetyl]amino}-1H-indol-2-yl)carbonyl]amino}-5-cyanobenzoic acid

5-cyano-2-{[(7-{[(4-methoxyphenyl)acetyl]amino}-1H-indol-2-yl)carbonyl]amino}benzoic acid

5-cyano-2-[({7-[(cyclopentylacetyl) amino] -1H-indol-2-yl}carbonyl)amino]benzoic acid

5-cyano-2-[({7-[(3-fluorobenzoyl)amino]-1H-indol-2-yl}carbonyl)amino]benzoic acid

5-cyano-2-[({7-[(3-cyanobenzoyl)amino]-1H-indol-2-yl}carbonyl)amino]benzoic acid

5-cyano-2-[({7-[(cyclohexylcarbonyl)amino]-1H-indol-2-yl}carbonyl)amino]benzoic acid

5-cyano-2-({[7-(propionylamino)-1H-indol-2-yl]carbonyl}amino)benzoic acid

5-cyano-2-[({7-[(5-methoxy-5-oxopentanoyl)amino]-1H-indol-2-yl}carbonyl)amino]ben acid

2-({[7-(butyrylamino)-1H-indol-2-yl]carbonyl}amino)-5-cyanobenzoic acid

2-[({7-[(4-bromobenzoyl)amino]-1H-indol-2-yl}carbonyl)amino]-5-cyanobenzoic acid

5-cyano-2-[({7-[(3-phenylpropanoyl)amino]-1H-indol-2-yl}carbonyl)amino]benzoic acid

5-cyano-2-[({7-[(phenoxyacetyl)amino]-1H-indol-2-yl}carbonyl)amino]benzoic acid

5-cyano-2-[({7-[(3-cyclopentylpropanoyl)amino]-1H-indol-2-yl}carbonyl)amino]benzoic acid

5-cyano-2-[({7-[(3-methoxy-3-oxopropanoyl) amino] -1H-indol-2-yl}carbonyl)amino]benzoic acid

5-cyano-2-[({7-[(2-ethylhexanoyl)amino]-1H-indol-2-yl}carbonyl)amino]benzoic acid

5-cyano-2-{[(7-{[(3,4-dimethoxyphenyl)acetyl]amino}-1H-indol-2-yl)carbonyl]amino}benzoic acid

5-cyano-2-[({7-[(3,5,5-trimethylhexanoyl)amino]-1H-indol-2-yl}carbonyl)amino]benzoic acid

5-cyano-2-[({7-[(cyclopropylcarbonyl)amino]-1H-indol-2-yl}carbonyl)amino]benzoic acid

5-cyano-2-[({7-[(methoxyacetyl)amino]-1H-indol-2-yl}carbonyl)amino]benzoic acid

5-cyano-2-[({7-[(3-methylbutanoyl)amino]-1H-indol-2-yl}carbonyl)amino]benzoic acid

5-cyano-2-({[7-(pentanoylamino)-1H-indol-2-yl]carbonyl}amino)benzoic acid

5-cyano-2-{[(7-{[(4,7,7-trimethyl-3-oxo-2-oxabicyclo[2.2.1]hept-1-yl)carbonyl]amino}-1H-indol-2-yl)carbonyl]amino}benzoic acid

2-{[(7-{[chloro(phenyl)acetyl]amino}-1H-indol-2-yl)carbonyl]amino}-5-cyanobenzoic acid

2-{[(7-{[(benzyloxy)acetyl]amiino}-1H-indol-2-yl)carbonyl]amino}-5-cyanobenzoic acid

5-cyano-2-[({7-[(3-ethoxy-3-oxopropanoyl)amino]-1H-indol-2-yl}carbonyl)amino]benzoic acid

2-[({7-[(1-adamantylcarbonyl)amino]-1H-indol-2-yl}carbonyl)amino]-5-cyanobenzoic acid

5-cyano-2-({[7-(hexanoylamino)-1H-indol-2-yl]carbonyl}amino)benzoic acid

5-cyano-2-{[(7-{[(2-phenylcyclopropyl)carbonyl]amino}-1H-indol-2-yl)carbonyl]amino}acid

5-cyano-2-[({7-[(2-phenylbutanoyl)anino]-1H-indol-2-yl}carbonyl)amino]benzoic acid

5-cyano-2-({[7-(heptanoylamino)-1H-indol-2-yl]carbonyl}amino)benzoic acid

2-{[(7-{[(acetyloxy)(phenyl)acetyl]amino}-1H-indol-2-yl)carbonyl]amino}-5-cyanobenzo acid

5-cyano-2-[({7-[(thien-2-ylcarbonyl)amino]-1H-indol-2-yl}carbonyl)amino]benzoic acid

5-cyano-2-[({7-[(2-methylbutanoyl)amino]-1H-indol-2-yl}carbonyl)amino]benzoic acid

5-cyano-2-[({7-[(8-methoxy-8-oxooctanoyl)amino]-1H-indol-2-yl}carbonyl)amino]benzoic acid

5-cyano-2-[({7-[(2-ethylbutanoyl)amino]-1H-indol-2-yl}carbonyl)amino]benzoic acid

5-cyano-2-({[7-(octanoylamino)-1H-indol-2-yl]carbonyl}amino)benzoic acid

5-cyano-2-[({7-[(cyclobutylcarbonyl)amino]-1H-indol-2-yl}carbonyl)amino]benzoic acid

5-cyano-2-({[7-(1,3-dioxo-1,3-dihydro-2H-isoindol-2-yl)-1H-indol-2-yl]carbonyl}amino)benzoic acid

2-({[7-({[2-(benzylthio)-1,3-thiazol-4-yl]carbonyl}amino)-1H-indol-2-yl]carbonyl}amino)-5-cyanobenzoic acid

5-cyano-2-{[(7-{[3-(morpholin-4-ylsulfonyl)benzoyl]amino)}-1H-indol-2-yl)carbonyl]amino}benzoic acid

5-cyano-2-[({7-[(1H-indol-2-ylcarbonyl)amino]-1H-indol-2-yl}carbonyl)amino]benzoic acid

5-cyano-2-{[(7-{[(1-methyl-1H-indol-2-yl)carbonyl]amino}-1H-indol-2-yl)carbonyl]amino}benzoic acid

5-cyano-2-{[(7-{[(5-phenylisoxazol-3-yl)carbonyl]amino}-1H-indol-2-yl)carbonyl]amino}benzoic acid

5-cyano-2-[({7-[(5-phenylpentanoyl)amino]-1H-indol-2-yl}carbonyl)amino]benzoic acid

5-cyano-2-[({7-[(4-phenylbutanoyl)amino]-1H-indol-2-yl}carbonyl)amino]benzoic acid

5-cyano-2-{[(7-{[4-(4-methoxyphenyl)butanoyl]amino)}-1H-indol-2-yl)carbonyl]amino}benzoic acid

5-cyano-2-[({6-[(1-methylbutyl)thio]pyiidin-3-yl}carbonyl)amino]benzoic acid

5-cyano-2-({[5-(3-methylthien-2-yl)isoxazol-3-yl]carbonyl}amino)benzoic acid

5-cyano-2-({[5-(3-methoxyphenyl)-1,3-oxazol-2-yl]carbonyl}amino)benzoic acid

2-{[(7-{[(2-chlorophenyl)acetyl]amino}-1H-indol-2-yl)carbonyl]amino}-5-cyanobenzoic acid

5-cyano-2-{[(7-{[(2,4-dichlorophenyl)acetyl]amino}-1H-indol-2-yl)carbonyl]amino}benzoic acid

5-cyano-2-{[(7-{[(3,4-dichlorophenyl)acetyl]amino}-1H-indol-2-yl)carbonyl]amino}benzoic acid

2-{[(7-{[(3-chlorophenyl)acetyl]anino}-1H-indol-2-yl)carbonyl]amino}-5-cyanobenzoic acid

5-cyano-2-({[7-({[3-(trifluoromethyl)phenyl]acetyl}amino)-1H-indol-2-yl]carbonyl}amino)benzoic acid

5-cyano-2-{[(7-{[(3-methylphenyl)acetyl]amino}-1H-indol-2-yl)carbonyl]amino}benzoic acid

2-{[(7-{[(4-tert-butylphenyl)acetyl]amino}-1H-indol-2-yl)carbonyl]amino}-5-cyanobenzoic acid

5-cyano-2-{[(7-{[(3-methoxyphenyl)acetyl]amino}-1H-indol-2-yl)carbonyl]amino}benzoic acid

5-cyano-2-{[(7-{[(2-methoxyphenyl)acetyl]amino}-1H-indol-2-yl)carbonyl]amino}benzoic acid

5-cyano-2-{[(7-{[(2-methylphenyl)acetyl]amino}-1H-indol-2-yl)carbonyl]amino}benzoic acid

5-cyano-2-({[7-({[4-(trifluoromethyl)phenyl]acetyl}amino)-1H-indol-2-yl]carbonyl}acid

5-cyano-2-{[(7-{[(4-isopropylphenyl)acetyl]amino}-1H-indol-2-yl)carbonyl]amino}benzoic acid

5-cyano-2-{[(7-{[(4-methylphenyl)acetyl]amino}-1H-indol-2-yl)carbonyl]amino}benzoic acid

5-cyano-2-{[(7-{[(4-fluorophenyl)acetyl]amino}-1H-indol-2-yl)carbonyl]amino}benzoic acid

2-({[5-(butylthio)pyrazin-2-yl]carbonyl}anino)-5-cyanobenzoic acid

5-cyano-2-({[7-({[2-(trifluoromethyl)phenyl]acetyl}amino)-1H-indol-2-yl]carbonyl}amino)benzoic acid

5-cyano-2-{[(7-{[(3-fluorophenyl)acetyl]amino}-1H-indol-2-yl)carbonyl]amino}benzoic acid

5-cyano-2-{[(7-{[(phenylthio)acetyl]amino}-1H-indol-2-yl)carbonyl]amino}benzoic acid

5-cyano-2-[({7-[(2-naphthylacetyl)amino]-1H-indol-2-yl}carbonyl)amino]benzoic acid

5-cyano-2-[({7-[(1-naphthylacetyl)amino]-1H-indol-2-yl}carbonyl)amino]benzoic acid

5-cyano-2-{[(7-{[(2-naphthyloxy)acetyl]amino}-1H-indol-2-yl)carbonyl]amino}benzoic acid

5-cyano-2-[({7-[(2-propoxybenzoyl)amino]-1H-indol-2-yl}carbonyl)amino]benzoic acid

5-cyano-2-[({7-[(tetrahydrofiuran-3-ylcarbonyl)amino]-1H-indol-2-yl}carbonyl)amino]benzo acid

5-cyano-2-{[(7-{[(1-methylcyclopropyl)carbonyl]amino}-1H-indol-2-yl)carbonyl]amino}benzoic acid

5-cyano-2-{[(7-{[(4-ethoxyphenyl)acetyl]amino}-1H-indol-2-yl)carbonyl]amino}benzoic acid

2-[({7-[(1-benzothien-3-ylacetyl)amino]-1H-indol-2-yl}carbonyl)amino]-5-cyanobezoic acid

2-[({7-[(1,1′-biphenyl-4-ylcarbonyl)amino]-1H-indol-2-yl}carbonyl)amino]-5-cyanobenzoic acid

2-[({7-[(4-butoxybenzoyl)amino]-1H-indol-2-yl}carbonyl)amino]-5-cyanobenzoic acid

5-cyano-2-{[(7-{[2-(2-phenylethyl)benzoyl]aminol}-1H-indol-2-yl)carbonyl]amino}benzoic acid

2-[({7-[(1,1 ′-biphenyl-2-ylcarbonyl)amino]-1H-indol-2-yl}carbonyl)amino]-5-cyanobenzoic acid

5-cyano-2-{[(7-{[4-(ethylthio)benzoyl]amino}-1H-indol-2-yl)carbonyl]amino}benzoic acid

5-cyano-2-{[(7-{[2-(methylsulfonyl)benzoyl]amino}-1H-indol-2-yl)carbonyl]amino}acid

5-cyano-2-{[(7-{[(2,6-dichlorophenyl)acetyl]amino}-1H-indol-2-yl)carbonyl]amino}acid

2-[({7-[(1,1′-biphenyl-4-ylacetyl)amino]-1H-indol-2-yl}carbonyl)amino]-5-cyanbenzoic acid

2-[({7-[(1,3-benzodioxol-5-ylacetyl)amino]-1H-indol-2-yl}carbonyl)amino]-5-cyanobenzoic acid

5-cyano-2-[({7-[(3,3-dimethylbutanoyl)amino]-1H-indol-2-yl}carbonyl)amino]benzoic acid

5-cyano-2-[({7-[(thien-2-ylacetyl)amino]-1H-indol-2-yl}carbonyl)amino]benzoic acid

5-bromo-2-({[5-(4-cyanophenyl)-1,3-oxazol-2-yl]carbonyl}amino)benzoic acid

5-bromo-2-{[(5-methoxypyrazin-2-yl)carbonyl]amino}benzoic acid

2-({[5-(1,3-benzodioxol-5-yl)-1,3-oxazol-2-yl]carbonyl}amino)-5-cyanobenzoic acid

5-bromo-2-({[5-(sec-butylthio)pyrazin-2-yl]carbonyl}amino)benzoic acid

5-bromo-2-({[5-(butylthio)pyrazin-2-yl]carbonyl}amino)benzoic acid

2-({[5-(butylthio)pyrazin-2-yl]carbonyl}amino)-5-chlorobenzoic acid

5-cyano-2-[(E)-2-(1-methyl-1H-indol-2-yl)ethenyl]benzoic acid

5-bromo-2-{[(1-methyl-3-phenyl-1H-pyrazol-5-yl)carbonyl]amino}benzoic acid

5-bromo-2-{[(1-methyl-5-phenyl-1H-pyrazol-3-yl)carbonyl]amino}benzoic acid

5-bromo-2-({[5-(4-methoxyphenyl)-1-methyl-1H-pyrazol-3-yl]carbonyl}amino)benzoic acid

5-bromo-2-{[(3-phenyl-1H-pyrazol-5-yl)carbonyl]amino}benzoic acid

2-[(1,2-benzisoxazol-3-ylcarbonyl)amino]-5-bromobenzoic acid

5-cyano-2-[({5-[2-(trifuoromethyl)phenyl]-1,3-oxazol-2-yl}carbonyl)amino]benzoic acid

2-[({6-[(4-aminobutyl)thio]pyridin-3-yl}carbonyl)amino]-5-cyanobenzoic acid trifluoroacetate

5-bromo-2-({[5-(pentylthio)pyrazin-2-yl]carbonyl}amino)benzoic acid

5-bromo-2-({[5-(hexylthio)pyrazin-2-yl]carbonyl}amino)benzoic acid

2-({[5-(sec-butylthio)pyrazin-2-yl]carbonyl}amino)-5-cyanobenzoic acid

5-cyano-2-({[5-(2-furyl)-1,3-oxazol-2-yl]carbonyl}amino)benzoic acid

5-bromo-2-({[5-(2-furyl)-1,3-oxazol-2-yl]carbonyl}amino)benzoic acid

5-bromo-2-({[3-(2,6-difluorophenyl)-1-methyl-1H-pyrazol-5-yl]carbonyl}amino)benzoic acid

5-cyano-2-{[(7-{[(3-methyl-5-phenylisoxazol-4-yl)carbonyl]amino}-1H-indol-2-yl)carbonyl]amino}benzoic acid

5-cyano-2-({[7-({[2-(2-methoxyethoxy)ethoxy]acetyl}amino)-1H-indol-2-yl]carbonyl}amino)benzoic acid

5-cyano-2-[({7-[(2-hydroxybenzoyl)amino]-1H-indol-2-yl}carbonyl)amino]benzoic acid

5-cyano-2-({[7-({[4-(trifluoromethoxy)phenyl]sulfonyl}amino)-1H-indol-2-yl]carbonyl}ami acid

5-cyano-2-({[7-(prolylamino)-1H-indol-2-yl]carbonyl}amino)benzoic acid

5-cyano-2-{[(7-{[(3-methylisoxazol-5-yl)acetyl]amino}-1H-indol-2-yl)carbonyl]amino}benzoic acid

5-bromo-2-({[5-(4-fluorophenyl)-1,3-oxazol-2-yl]carbonyl}amino)benzoic acid

5-cyano-2-({[5-(4-cyanophenyl)-1,3-oxazol-2-yl]carbonyl}amino)benzoic acid

5-bromo-2-({[5-(1-methyl-1H-pyrrol-2-yl)-1,3-oxazol-2-yl]carbonyl}amino)benzoic acid

5-bromo-2-({[5-(3-cyanophenyl)-1,3-oxazol-2-yl]carbonyl}amino)benzoic acid

5-cyano-2-({[5-(pentylthio)pyrazin-2-yl]carbonyl}amino)benzoic acid

5-bromo-2-{[(5-{[3-(2-methoxyethoxy)propyl]thio}pyrazin-2-yl)carbonyl]amino}benzoic acid

5-chloro-2-{[(5-{[3-(2-methoxyethoxy)propyl]thio}pyrazin-2-yl)carbonyl]amino}benzoic acid

5-cyano-2-[({5-[2-({[4-(trifluoromethoxy)phenyl]sulfonyl}amino)phenyl]isoxazol-3-acyl}carbonyl)amino]benzoic

5-chloro-2-[({5-[2-({[4-(trifluoromethoxy)phenyl]sulfonyl}amino)phenyl]isoxazol-3-yl}carbonyl)amino]benzoic acid

2-({[5-(1,3-benzodioxol-5-yl)isoxazol-3-yl]carbonyl}amino)-5-cyanobenzoic acid

2-({[5-(1,3-benzodioxol-5-yl)isoxazol-3-yl]carbonyl}amino)-5-bromobenzoic acid

5-cyano-2-{[(5-nitro-1,2-benzisoxazol-3-yl)carbonyl]amino}benzoic acid

2-{[(1-acetyl-1H-indazol-3-yl)carbonyl]amino}-5-cyanobenzoic acid

2-[(1,3-benzoxazol-2-ylcarbonyl)amino]-5-cyanobenzoic acid

2-[({7-[(benzylsulfonyl)amino]-1H-indol-2-yl}carbonyl)amino]-5-cyanobenzoic acid

5-cyano-2-{[(1-methyl-7-{[3-(morpholin-4-ylsulfonyl)benzoyl]amino}-1H-indol-2-yl)carbonyl]amino}benzoic acid

5-cyano-2-{[(7-{[(4-fluorophenyl)acetyl]amino}-1-methyl-1H-indol-2-yl)carbonyl]amino}acid

5-cyano-2-[({7-[(fluoroacetyl)amino]-1-methyl-1H-indol-2-yl}carbonyl)amino]benzoic acid

5-cyano-2-{[(1-methyl-7-{[(1-methyl-1H-indol-2-yl)carbonyl]aminol}-1H-indol-2-yl)carbonyl]amino}benzoic acid

5-cyano-2-{[(5-{[3-(2-methoxyethoxy)propyl]thio}pyrazin-2-yl)carbonyl]amino}benzoic acid

5-chloro-2-({[5-(pentylthio)pyrazin-2-yl]carbonyl}anino)benzoic acid

5-cyano-2-({[5-(hexylthio)pyrazin-2-yl]carbonyl}amino)benzoic acid

5-chloro-2-({[5-(hexylthio)pyrazin-2-yl]carbonyl}amino)benzoic acid

5-cyano-2-({[5-(1-methyl-1H-pyrrol-2-yl)-1,3-oxazol-2-yl]carbonyl}amino)benzoic acid

2-({[5-(sec-butylthio)pyrazin-2-yl]carbonyl}amino)-5-chlorobenzoic acid

5-cyano-2-({[5-(3-cyanophenyl)-1,3-oxazol-2-yl]carbonyl}amino)benzoic acid

5-bromo-2-[({2-[(3R)-3,4-dihydroxybutyl]-1,3-oxazol-4-yl}carbonyl)amino]benzoic acid

5-cyano-2-[({5-[(phenylacetyl)amino]-1,2-benzisoxazol-3-yl}carbonyl)amino]benzoic acid

2-[({5-[(benzylsulfonyl)amino]-1,2-benzisoxazol-3-yl}carbonyl)amino]-5-cyanobenzoic acid

2-({[6-(benzyloxy)-1-methyl-1H-indol-2-yl]carbonyl}amino)-5-cyanobenzoic acid

5-cyano-2-{[(6-methoxy-1-methyl-1H-indol-2-yl)carbonyl]amino}benzoic acid

5-cyano-2-({[5-(2-isopropoxyphenyl)isoxazol-3-yl]carbonyl}amino)benzoic acid

5-cyano-2-[({1-methyl-7-[(morpholin-4-ylcarbonyl)amino]-1H-indol-2-yl}carbonyl)am acid

5-cyano-2-({[1-methyl-7-({[(tetrahydrofuran-2-ylnethyl)amino]carbonyl}amino)-1H-indol-2-yl]carbonyl}amino)benzoic amino)benzoic acid

5-cyano-2-{[(7-hydroxy-1-methyl-1H-indol-2-yl)carbonyl]amino}benzoic acid

5-bromo-2-{[(2-{2-[(4S)-2,2-dimethyl-1,3-dioxolan-4-yl]ethyl}-1,3-oxazol-4-yl)carbonyl]amino}benzoic acid

5-cyano-2-({[5-(2-phenylethyl)pyrazin-2-yl]carbonyl}amino)benzoic acid

5-bromo-2-{[(5-{(E)-2-[(4S)-2,2-dimethyl-1,3-dioxolan-4-yl]ethenyl}pyrazin-2-yl)carbonyl]amino}benzoic acid

5-cyano-2-({[5-(isopentylthio)pyrazin-2-yl]carbonyl}amino)benzoic acid

5-cyano-2-({[5-(isobutylthio)pyrazin-2-yl]carbonyl}amino)benzoic acid

5-cyano-2-{[(5-methoxypyrazin-2-yl)carbonyl]amino}benzoic acid

2-{[(7-{[(benzylamino)carbonyl]amino}-1-methyl-1H-indol-2-yl)carbonyl]amino}-5acid

5-cyano-2-({[7-({[(2,3-dihydroxypropyl)amino]carbonyl}amino)-1-methyl-1H-indol-2-yl]carbonyl}amino)benzoic acid

1-[{[(2-{[(2-carboxy-4-cyanophenyl)amino]carbonyl}-1-methyl-1H-indol-7-yl)amino]carbonyl}(methyl)amino]-1-deoxyhexitol

5-cyano-2-({[7-(2,3-dihydro-1,4-benzodioxin-2-ylmethoxy)-1-methyl-1H-indol-2-yl]carbonyl}amino)benzoic acid

5-cyano-2-{[(5-{2-[2-(2-ethoxyethoxy)ethoxy]phenyl}isoxazol-3-yl)carbonyl]amino}benzoic acid

5-cyano-2-[({5-[2-(hexyloxy)phenyl]isoxazol-3-yl}carbonyl)amino]benzoic acid

2-[({5-[2-(allyloxy)phenyl]isoxazol-3-yl}carbonyl)amino]-5-cyanobenzoic acid

5-cyano-2-[({⁵-[2-(2,3-dihydro-1,4-benzodioxin-2-ylmethoxy)phenyl]isoxazol-3-yl}carbonyl)amino]benzoic acid

2-({[7-(benzyloxy)-1H-indol-2-yl]carbonyl}amino)-5-cyanobenzoic acid

5-cyano-2-({[1-methyl-7-(3-phenoxypropoxy)-1H-indol-2-yl]carbonyl}anino)benzoic acid

5-cyano-2-({[5-(hydroxymethyl)isoxazol-3-yl]carbonyl}amino)benzoic acid

5-cyano-2-({[5-(hexyloxy)pyrazin-2-yl]carbonyl}amino)benzoic acid

2-({[5-(acetylamino)-1,2-benzisoxazol-3-yl]carbonyl}anino)-5-cyanobenzoic acid

5-cyano-2-[({5-[(methylsulfonyl)amino]-1,2-benzisoxazol-3-yl}carbonyl)amino]benzoic acid

5-bromo-2-[({5-[2-(2,2-dimethyl-1,3-dioxolan-4-yl)ethyl]isoxazol-3-yl}carbonyl)amino]benzoic acid

2-({[5-(benzoylamino)-1,2-benzisoxazol-3-yl]carbonyl}amino)-5-cyanobenzoic acid

5-cyano-2-[({5-[(phenylsulfonyl)amino]-1,2-benzisoxazol-3-yl}carbonyl)amino]benzoic acid

5-cyano-2-({[5-(2-ethoxyphenyl)isoxazol-3-yl]carbonyl}amino)benzoic acid

5-cyano-2-[({5-[2-(tetrahydro-2H-pyran-2-ylmethoxy)phenyl]isoxazol-3-yl}carbonyl)amino acid

5-cyano-2-({[7-(cyclobutylmethoxy)-1-methyl-1H-indol-2-yl]carbonyl}anino)benzoic acid

5-cyano-2-[({5-[2-(3-phenoxypropoxy)phenyl]isoxazol-3-yl}carbonyl)amino]benzoic acid

5-cyano-2-({[7-(2-furylmethoxy)-1-methyl-1H-indol-2-yl]carbonyl}amino)benzoic acid

5-cyano-2-{[(7-{[(4S)-2,2-dimethyl-1,3-dioxolan-4-yl]methoxy}-1-methyl-1H-indol-2-yl)carbonyl]amino}benzoic acid

5-cyano-2-{[(7-{[(2R)-2,3-dihydroxypropyl]oxy}-1-methyl-1H-indol-2-yl)carbonyl]amino}benzoic acid

5-cyano-2-({[5-(2-hydroxyphenyl)isoxazol-3-yl]carbonyl}amino)benzoic acid

5-cyano-2-[({5-[2-(tetrahydrofuran-3-yloxy)phenyl]isoxazol-3-yl}carbonyl)amino]benzoic acid

5-cyano-2-[({5-[2-(cyclobutyloxy)phenyl]isoxazol-3-yl}carbonyl)amino]benzoic acid

5-cyano-2-[({5-[2-(tetrahydro-2H-pyran-4-yloxy)phenyl]isoxazol-3-yl}carbonyl)amino]benzoic acid

5-cyano-2-({[5-(2-{[(1S,2R)-2-methylcyclopentyl]oxy}phenyl)isoxazol-3-yl]carbonyl}amino)benzoic acid

5-cyano-2-[({5-[2-(2-methoxy-1-methylethoxy)phenyl]isoxazol-3-yl}carbonyl)amino]benzoic acid

5-cyano-2-[({5-[2-(1-methylbutoxy)phenyl]isoxazol-3-yl}carbonyl)amino]benzoic acid

5-cyano-2-[({5-[2-(cyclobutylmethoxy)phenyl]isoxazol-3-yl}carbonyl)amino]benzoic acid

5-cyano-2-({[7-(cyclobutyloxy)-1-methyl-1H-indol-2-yl]carbonyl}anino)benzoic acid

5-cyano-2-({[7-(2-methoxy-1-methylethoxy)-1-methyl-1H-indol-2-yl] carbonyl}amino)benzoic acid

5-cyano-2-{[(7-isopropoxy-1-methyl-1H-indol-2-yl)carbonyl]amino}benzoic acid

2-({[7-(benzyloxy)-1-methyl-1H-indol-2-yl]carbonyl}amino)-5-cyanobenzoic acid

2-{[(6-chloro-1,2-benzisoxazol-3-yl)carbonyl]amino}-5-cyanobenzoic acid

5-bromo-2-{[(6-chloro-1,2-benzisoxazol-3-yl)carbonyl]amino}benzoic acid

5-cyano-2-[({5-[2-(cyclohex-2-en-1-ylmethoxy)phenyl]isoxazol-3-yl}carbonyl)amino]benzoic acid

2-({[2-(3-{[(2-carboxy-4-cyanophenyl)amino]carbonyl}isoxazol-5-yl)phenoxy]acetyl}amino]acid

5-cyano-2-[({5-[2-(1-ethylpropoxy)phenyl]isoxazol-3-yl}carbonyl)amino]benzoic acid

5-cyano-2-{[(5-{2-[2-(4-methyl-1,3-thiazol-5-yl)ethoxy]phenyl}isoxazol-3-yl)carbonyl]amino}benzoic acid

5-cyano-2-[({5-[2-(1-cyclohexylethoxy)phenyl]isoxazol-3-yl}carbonyl)amino]benzoic acid 2-{[(6-sec-butoxy-1-methyl-1H-indol-2-yl)carbonyl]amino}-5-cyanobenzoic acid 2-{[(6-butoxy-1-methyl-1H-indol-2-yl)carbonyl]amino}-5-cyanobenzoic acid

5-cyano-2-{[(5-{[(4-fluorophenyl)sulfonyl]amino}-1,2-benzisoxazol-3-yl)carbonyl]amino}benzoic acid

5-cyano-2-[({5-[2-(trifluoromethyl)phenyl]pyrazin-2-yl}carbonyl)amino]benzoic acid

5-cyano-2-[({5-[2-(2-oxobutoxy)phenyl]isoxazol-3-yl}carbonyl)amino]benzoic acid

5-cyano-2-[({5-[2-(2-oxo-2-phenylethoxy)phenyl]isoxazol-3-yl}carbonyl)amino]benzoic acid

2-({[5-(2-bromophenyl)isoxazol-3-yl]carbonyl}amino)-5-cyanobenzoic acid

2-({[5-(1,1′-biphenyl-2-yl)isoxazol-3-yl]carbonyl}amino)-5-cyanobenzoic acid

2-{[(5-amino-1,2-benzisoxazol-3-yl)carbonyl]amino}-5-cyanobenzoic acid

5-cyano-2-[({5-[(4-methoxybenzyl)thio]pyrazin-2-yl}carbonyl)amino]benzoic acid

5-cyano-2-({[5-(3-methoxyphenyl)isoxazol-3-yl]carbonyl}anino)benzoic acid

5-cyano-2-({[5-(2-fluorophenyl)pyrazin-2-yl]carbonyl}amino)benzoic acid

5-bromo-2-{[(5-{(E)-2-[(2S)-1,4-dioxaspiro[4.5]dec-2-yl]ethenyl}pyrazin-2-yl)carbonyl]amino}benzoic acid

2-{[(5-bromo-1,2-benzisoxazol-3-yl)carbonyl]amino}-5-cyanobenzoic acid

2-[({6-[(benzylsulfonyl)amino]-1,2-benzisoxazol-3-yl}carbonyl)amino]-5-cyanobenzoic acid

5-cyano-2-[({6-[(phenylsulfonyl)amino]-1,2-benzisoxazol-3-yl}carbonyl)amino]benzoic acid

5-cyano-2-({[7-(cyclohexylmethoxy)-1-methyl-1H-indol-2-yl]carbonyl}amino)benzoic acid

5-cyano-2-({[7-(cyclopropylmethoxy)-1-methyl-1H-indol-2-yl]carbonyl}amino)benzoic acid

5-cyano-2-({[1-methyl-7-(tetrahydro-2H-pyran-2-ylmethoxy)-1H-indol-2-yl]carbonyl}amino)benzoic acid

5-cyano-2-({[1-methyl-7-(pentyloxy)-1H-indol-2-yl]carbonyl}amino)benzoic acid

2-({[5-(chloromethyl)isoxazol-3-yl]carbonyl}amino)-5-cyanobenzoic acid

5-cyano-2-({[5-(morpholin-4-ylmethyl)isoxazol-3-yl]carbonyl}amino)benzoic acid

5-cyano-2-{[(5-phenyl-1,2-benzisoxazol-3-yl)carbonyl]amino}benzoic acid

5-bromo-2-{[(5-bromo-1,2-benzisoxazol-3-yl)carbonyl]amino}benzoic acid

2-{[(5-{2-[(5-chloropentyl)oxy]phenyl}isoxazol-3-yl)carbonyl]amino}-5-cyan acid

2-({[4,5-bis(methoxymethyl)isoxazol-3-yl]carbonyl}amino)-5-cyanobenzoic acid

5-cyano-2-[({5-[2-(trifluoromethyl)phenyl]-1,2-benzisoxazol-3-yl}carbonyl)amino]benzoic acid

5-cyano-2-({[5-(2-methylphenyl)pyrazin-2-yl]carbonyl}amino)benzoic acid

5-cyano-2-({[5-(2,3,4-trimethoxyphenyl)pyrazin-2-yl]carbonyl}amino)benzoic acid

5-cyano-2-({[7-(2-methoxyethoxy)-1-methyl-1H-indol-2-yl]carbonyl}amino)benzoic acid

5-cyano-2-({[7-(2-hydroxy-3-isopropoxypropoxy)-1-methyl-1H-indol-2-yl]carbonyl}amino)benzoic acid

5-cyano-2-{[(6-phenyl-1,2-benzisoxazol-3-yl)carbonyl]amino}benzoic acid

5-cyano-2-[({6-[2-(trifluoromethyl)phenyl]-1,2-benzisoxazol-3-yl}carbonyl)amino]benzoic acid

2-[({5-[(benzylamino)methyl]isoxazol-3-yl}carbonyl)amino]-5-cyanobenzoic acid

2-{[(5-{[bis(2-hydroxyethyl)amino]methyl}isoxazol-3-yl)carbonyl]amino}-5-cyanobenzoic acid

2-({[5-(azidomethyl)isoxazol-3-yl]carbonyl}amino)-5-cyanobenzoic acid

5-cyano-2-({[5-(nonylthio)pyrazin-2-yl]carbonyl}amino)benzoic acid

5-cyano-2-[({1-methyl-7-[2-(methylthio)ethoxy]-1H-indol-2-yl}carbonyl)amino]benzoic acid

5-cyano-2-{[(5-phenyl-4,5-dihydroisoxazol-3-yl)carbonyl]amino}benzoic acid

5-cyano-2-[(4,5-dihydronaphtho[2,1-d]isoxazol-3-ylcarbonyl)amino]benzoic acid

5-cyano-2-[(pyrrolo[1,2-c]pyrimidin-3-ylcarbonyl)amino]benzoic acid

2-[({7-[(4-azido-3-iodobenzoyl)amino]-1-methyl-1H-indol-2-yl}carbonyl)amino]-5-cyanobenzoic acid

2-[({5-[(4-azido-3-iodobenzoyl)amino]-1,2-benzisoxazol-3-yl}carbonyl)amino]-5-cyanobenzoic acid

2-[(E)-2-(1,2-benzisoxazol-3-yl)ethenyl]-5-cyanobenzoic acid

5-cyano-2-{[(5-{[(pyridin-4-ylmethyl)amino]methyl}isoxazol-3-yl)carbonyl]anino}benzoic acid trifluoroacetate

5-cyano-2-[({5-[(pyridin-4-ylthio)methyl]isoxazol-3-yl}carbonyl)amino]benzoic acid

5-cyano-2-({[6-(hexylthio)pyridazin-3-yl]carbonyl}amino)benzoic acid

5-cyano-2-({[5-(octylthio)pyrazin-2-yl]carbonyl}amino)benzoic acid

5-cyano-2-({[5-(6-methoxypyridin-3-yl)pyrazin-2-yl]carbonyl}amino)benzoic acid

5-cyano-2-{[(5-phenylpyrazin-2-yl)carbonyl]amino}benzoic acid

5-cyano-2-[(4,5,6,7-tetrahydro-1,2-benzisoxazol-3-ylcarbonyl)amino]benzoic acid

5-cyano-2-[({5-[4-(methylsulfonyl)phenyl]pyrazin-2-yl}carbonyl)amino]benzoic acid

5-cyano-2-({[5-(3,5-dimethylisoxazol-4-yl)pyrazin-2-yl]carbonyl}amino)benzoic acid

2-[(2, I -benzisoxazol-3-ylcarbonyl)amino]-5-cyanobenzoic acid

2-({[5-({[amino(imino)methyl]amino}methyl)isoxazol-3-yl]carbonyl}amino)-5-cyanobenzoic acid

2-[(2,1-benzisoxazol-3-ylcarbonyl)amino]-5-bromobenzoic acid

5-cyano-2-[({6-[(methylsulfonyl)amino]-1,2-benzisoxazol-3-yl}carbonyl)amino]benzoic acid

5-cyano-2-{[(6-{[(4-fluorophenyl)sulfonyl]amino}-1,2-benzisoxazol-3-yl)carbonyl]amino}benzoic acid

2-{[(6-amino-1,2-benzisoxazol-3-yl)carbonyl]amino}-5-cyanobenzoic acid

5-cyano-2-({[5-(2-fluorophenyl)-1,2-benzisoxazol-3-yl]carbonyl}amino)benzoic acid

5-cyano-2-[({6-[2-(trifluoromethyl)phenyl]pyridazin-3-yl}carbonyl)amino]benzoic acid

2-{[(2-benzyl-1,1-dioxido-3,4-dihydro-2H-1,2-benzothiazin-5-yl)carbonyl]amino}-5-bromobenzoic acid

2-{[(2-benzyl-1,1-dioxido-3,4-dihydro-2H-1,2-benzothiazin-5-yl)carbonyl]amino}-5-cyanobenzoic acid

5-cyano-2-[({1-methyl-6-[2-(trifluoromethyl)phenyl]-1H-indol-2-yl}carbonyl)amino]benzoic acid

5-cyano-2-({([1-methyl-6-(2,3,4-trimethoxyphenyl)-1H-indol-2-yl]carbonyl}amino)benzoi acid

5-iodo-2-{[(1-methyl-1H-indol-2-yl)carbonyl]amino}benzoic acid

5-iodo-2-({([1-methyl-7-(phenylsulfonyl)-1H-indol-2-yl]carbonyl}amino)benzoic acid

5-cyano-2-({[1-methyl-7-(pyridin-3-ylmethoxy)-1H-indol-2-yl]carbonyl}amino)benzoic acid

2-({[5-(2-fluorophenyl)isoxazol-3-yl]carbonyl}amino)-5-iodobenzoic acid

5-iodo-2-({[5-(2-methylphenyl)isoxazol-3-yl]carbonyl}amino)benzoic acid

5-iodo-2-{[(5-phenylisoxazol-3-yl)carbonyl]amino}benzoic acid

4-({[5-(2-fluorophenyl)isoxazol-3-yl]carbonyl}amino)-2′-(trifluoromethyl)-1,1′-biphenyl-3-carboxylic acid

2-({[5-(2-{[(2R)-3-bromo-2-methylpropyl]oxy}phenyl)isoxazol-3-yl]carbonyl}aami acid

5-cyano-2-({[6-(heptylthio)pyridazin-3-yl]carbonyl}amino)benzoic acid

5-cyano-2-({[5-(heptylthio)pyrazin-2-yl]carbonyl}amino)benzoic acid

5-cyano-2-({[6-(3,5-dimethylisoxazol-4-yl)-1-methyl-1H-indol-2-yl]carbonyl}amino)benzoic acid

2-{[(5-chloropyrazin-2-yl)carbonyl]amino}-5-cyanobenzoic acid

5-cyano-2-({[1-methyl-6-(2-methylphenyl)-1H-indol-2-yl]carbonyl}amino)benzoic acid

5-bromo-2-({[6-(hexylthio)pyridazin-3-yl] carbonyl}amino)benzoic acid

5-cyano-2-({[6-(pentylthio)pyridazin-3-yl]carbonyl}amino)benzoic acid

2-{[(5-chloropyrazin-2-yl)carbonyl]amino}-5-iodobenzoic acid

2-({[5-(benzylthio)pyrazin-2-yl]carbonyl}anino)-5-iodobenzoic acid

2-[({5-[(benzyloxy)carbonyl]-4,5,6,7-tetrahydroisoxazolo[4,5-c]pyridin-3-yl}carbonyl)amino]-5-cyanobenzoic acid

2-{[(7-amino-4,6-dibromo-1-methyl-1H-indol-2-yl)carbonyl]amino}-5-cyanobenzoic acid

5-cyano-2-({[5-(2-fluorophenyl)isoxazol-3-yl]carbonothioyl}amino)benzoic acid

2-[({5-[2-(1,1′-biphenyl-4-yloxy)phenyl]isoxazol-3-yl}carbonyl)amino] -5-cyan acid

2-[({5-[2-(1,1′-biphenyl-3-yloxy)phenyl]isoxazol-3-yl}carbonyl)amino]-5-cyanobenzoic acid

5-cyano-2-({[5-(3,4-dihydroxybutyl)isoxazol-3-yl]carbonyl}amino)benzoic acid

5-cyano-2-[({4,6-dibromo-1-methyl-7-[(phenylacetyl)amino]-1H-indol-2-yl}carbonyl)amino]benzoic acid

2-({[6-(acetyloxy)-1,2-benzisoxazol-3-yl]carbonyl}amino)-5-cyanobenzoic acid

5-cyano-2-{[(6-hydroxy-1,2-benzisoxazol-3-yl)carbonyl]amino}benzoic acid

2-[({5-[2-(1,1′-biphenyl-2-yloxy)phenyl]isoxazol-3-yl}carbonyl)amino]-5-cyanobenzoic acid

5-cyano-2-({[5-(hexylthio)pyrimidin-2-yl]carbonyl}amino)benzoic acid

2-({[6-(benzyloxy)-1,2-benzisoxazol-3-yl]carbonyl}amino)-5-cyanobenzoic acid

5-cyano-2-{[(6-methoxy-1,2-benzisoxazol-3-yl)carbonyl]amino}benzoic acid

2-[({6-[(benzylsulfonyl)oxy]-1,2-benzisoxazol-3-yl}carbonyl)amino]-5-cyanobenzoic acid

5-cyano-2-[({6-[(phenylsulfonyl)oxy]-1,2-benzisoxazol-3-yl}carbonyl)amino]benzoic acid

2-{[(7-amino-4-bromo-1-methyl-1H-indol-2-yl)carbonyl]amino}-5-cyanobenzoic acid

2-({[7-(benzyloxy)-3-bromo-1H-indol-2-yl]carbonyl}amino)-5-cyanobenzoic acid

2-[(1H-benzirnidazol-2-ylcarbonyl)amino]-5-cyanobenzoic acid

5-bromo-2-{[(5-{2-[(2R)-1,4-dioxaspiro[4. 5]dec-2-yl]-2-hydroxyethyl}pyrazin-2-yl)carbonyl]amino}benzoic acid

2-{[(6-{[(acetyloxy)acetyl]amino}-1-methyl-1H-indol-2-yl)carbonyl]amino}-5-cyanobenzoic acid

2-({[5-(anilinosulfonyl)-1,2-benzisoxazol-3-yl]carbonyl}amino)-5-cyanobenzoic acid

5-cyano-2-[({5-[(diethylamino)sulfonyl]-1,2-benzisoxazol-3-yl}carbonyl)amino]benzoic acid

2-{[(7-amino-6-bromo-1-methyl-1H-indol-2-yl)carbonyl]amino}-5-cyanobenzoic acid

5-cyano-2-[({1-methyl-7-[(phenylacetyl)amino]-4,6-bis[2-(trifluoromethyl)phenyl]-1H-indol-2-yl}carbonyl)amino]benzoic acid

5-bromo-2-({[6-(heptylthio)pyridazin-3-yl] carbonyl}amino)benzoic acid

5-cyano-2-[({5-nitro-6-[4-nitro-2-(trifluoromethyl)phenyl]-1,2-benzisoxazol-3-yl}carbonyl)amino]benzoic acid

2-({[7-(benzyloxy)-3-bromo-1-methyl-1H-indol-2-yl]carbonyl}amino)-5-cyanobenzoic acid

2-[({4-bromo-1-methyl-7-[(phenylacetyl)amino]-1H-indol-2-yl}carbonyl)amino]-5-cyanobenzoic acid

5-cyano-2-[({1-methyl-6-[(quinolin-8-ylsulfonyl)amino]-1H-indol-2-yl}carbonyl)amino]benzoic acid

5-cyano-2-({[5-(morpholin-4-ylsulfonyl)-1,2-benzisoxazol-3-yl]carbonyl}amino)benzoic acid

5-cyano-2-({[6-(cyclobutylmethoxy)-1-methyl-1H-indol-2-yl]carbonyl}amino)benzoic acid

2-({[6-(butyrylamino)-1-methyl-1H-indol-2-yl]carbonyl}anino)-5-cyanobenzoic acid

5-cyano-2-[({6-[(methoxyacetyl)amino]-1-methyl-1H-indol-2-yl}carbonyl)amino]benzoic acid

5-bromo-2-({[6-(pentylthio)pyridazin-3-yl]carbonyl}amino)benzoic acid

5-cyano-2-{[(7-methyl-1H-indol-2-yl)carbonyl]amino}benzoic acid

5-cyano-2-{[(6-oxo-5,6-dihydro-4H-pyrrolo[3,2,1-ij]quinolin-2-yl)carbonyl]amino}benzoic acid

2-[({5-(acetylamino)-6-[4-(acetylamino)-2-(trifluoromethyl)phenyl]-1,2-benzisoxazol-3-yl}carbonyl)amino]-5-cyanobenzoic acid

5-cyano-2-[({5-[(dimethylamino)sulfonyl]-1,2-benzisoxazol-3-yl}carbonyl)amino]benzoic acid

5-cyano-2-[({5-[(methoxyacetyl)amino]-1,2-benzisoxazol-3-yl}carbonyl)amino]benzoic acid

5-cyano-2-[({5-[(cyclobutylcarbonyl)amino]-1,2-benzisoxazol-3-yl}carbonyl)amino]benzoic acid

5-cyano-2-({[6-(2-fiuroylamino)-1-methyl-1H-indol-2-yl]carbonyl}amino)benzoic acid

5-cyano-2-({[1-methyl-6-({[methyl(phenyl)amino]carbonyl}amino)-1H-indol-2-yl]carbonyl}amino)benzoic acid

2-({[6-(acetylamino)-1-methyl-1H-indol-2-yl]carbonyl}amino)-5-cyanobenzoic acid

5-cyano-2-{[(1-methyl-6-{[4-(trifluoromethoxy)benzoyl]amino}-1H-indol-2-yl)carbonyl]amino}benzoic acid

5-cyano-2-({[6-(2-fluorophenyl)-1-methyl-1H-indol-2-yl]carbonyl}amino)benzoic acid

5-cyano-2-({[6-(cyclopropylmethoxy)-1-methyl-1H-indol-2-yl]carbonyl}amino)benzoic acid

5-cyano-2-{[(i-methyl-1H-indol-3-yl)carbonyl]amino}benzoic acid

2-{[(7-bromo-1-methyl-1H-indol-2-yl)carbonyl]amino}-5-cyanobenzoic acid

5-bromo-2-{[(5-{2-[(2R)-1,4-dioxaspiro[4. 5]dec-2-yl]-2-methoxyethyl}pyrazin-2-yl)carbonyl]amino}benzoic acid

5-bromo-2-[({5-[(3R)-2,3,4-trihydroxybutyl]pyrazin-2-yl}carbonyl)amino]benzoic acid

5-cyano-2-{[(6-methoxy-5,6-dihydro-4H-pyrrolo[3,2,1-ij]quinolin-2-yl)carbonyl]amino}benzoic acid

5-cyano-2-{[(6-hydroxy-5,6-dihydro-4H-pyrrolo[3,2,1-ij]quinolin-2-yl)carbonyl]amino}benzoic acid

5-cyano-2-{[(6-phenoxy-5,6-dihydro-4H-pyrrolo[3,2,1-ij]quinolin-2-yl)carbonyl]amino}benzoic acid

5-cyano-2-[({1-methyl-7-[2-(trifluoromethyl)phenyl]-1H-indol-2-yl}carbonyl)amino acid

5-cyano-2-{[(1-methyl-7-phenyl-1H-indol-2-yl)carbonyl]amino}benzoic acid

2-({[7-(4-tert-butylphenyl)-1-methyl-1H-indol-2-yl]carbonyl}amino)-5-cyanobenzoic acid

5-cyano-2-({[1-methyl-7-(5-methylthien-2-yl)-1H-indol-2-yl]carbonyl}amino)benzoic acid

5-cyano-2-[({1-methyl-6-[(methylsulfonyl)amino]-1H-indol-2-yl}carbonyl)amino]benzoic acid

5-bromo-2-[({5-[(3R)-2,3,4-trimethoxybutyl]pyrazin-2-yl}carbonyl)amino]benzoic acid

5-bromo-2-[({5-[(3R)-3,4-dihydroxy-2-methoxybutyl]pyrazin-2-yl}carbonyl)amino]benzoic acid

2-[({5-[3,4-bis(methoxymethoxy)butyl]isoxazol-3-yl}carbonyl)amino]-5-bromobenzoic acid

5-cyano-2-({[6-({[(2,5-dimethoxyphenyl)amino]carbonyl}amino)-1-methyl-1H-indol-2-yl]carbonyl}amino)benzoic acid

5-cyano-2-[({6-[(isoxazol-5-ylcarbonyl)amino]-1-methyl-1H-indol-2-yl}carbonyi)amino]benzoic acid

5-cyano-2-{[(1-methyl-6-{[(pentylamino)carbonyl] aminol}-1H-indol-2-yl)carbonyl]amino}benzoic acid

5-cyano-2-[({1-methyl-7-[4-(methylsulfonyl)phenyl]-1H-indol-2-yl}carbonyl)amino]benzoic acid

5-cyano-2-({[7-(2-methoxyphenyl)-1-methyl-1H-indol-2-yl]carbonyl}anino)benzoic acid

5-cyano-2-({[7-(2-fluorophenyl)-1-methyl-1H-indol-2-yl]carbonyl}amino)benzoic acid

5-cyano-2-({[1-methyl-7-(2-methylphenyl)-1H-indol-2-yl]carbonyl}amino)benzoic acid

5-cyano-2-[({6-[(pyridin-4-ylmethyl)thio]pyridazin-3-yl}carbonyl)anino]benzoic acid

5-cyano-2-{[(6-{[(3,5-dimethylisoxazol-4-yl)sulfonyl]amino}-1-methyl-1H-indol-2-yl_carbonyl]amino}benzoic acid

5-cyano-2-({[5-(dimethylamino)-1,2-benzisoxazol-3-yl]carbonyl}amino)benzoic acid

5-cyano-2-({[5-(ethylamino)-1,2-benzisoxazol-3-yl]carbonyl}amino)benzoic acid

5-cyano-2-[({5-[(cyclopropylmethyl)amino]-1,2-benzisoxazol-3-yl}carbonyl)amino]benzoic acid

5-cyano-2-[({5-[(2-methoxyethyl)amino]-1,2-benzisoxazol-3-yl}carbonyl)amino]benzoic acid

5-cyano-2-[({5-[(2-hydroxyethyl)amino]-1,2-benzisoxazol-3-yl}carbonyl)amino]benzoic acid

5-cyano-2-[({⁵-[(2,3-dihydroxypropyl)amino]-1,2-benzisoxazol-3-yl}carbonyl)amino]benzoic acid

5-bromo-2-[({5-[(3R)-2-methoxy-3,4-bis(methoxymethoxy)butyl]pyrazin-2-yl}carbonyl)amino]benzoic acid

2-[({6-[bis(butylsulfonyl)amino]-1-methyl-1H-indol-2-yl}carbonyl)amino]-5-cyanobenzoic acid

2-[({6-[bis(phenylsulfonyl)amino]-1-methyl-1H-indol-2-yl}carbonyl)amino]-5-cyanobenzoic acid

5-cyano-2-{[(6-{[(2-methoxyethoxy)acetyl]amino}-1-methyl-1H-indol-2-yl)carbonyl]amino}benzoic acid

5-cyano-2-[(1H-indazol-3-ylcarbonyl)amino]benzoic acid

5-cyano-2-({[5-(hexylthio)pyridin-2-yl]carbonyl}amino)benzoic acid

5-cyano-2-{[(6-{[(2E)-5-hydroxypent-2-enyl]thio}pyridazin-3-yl)carbonyl]amino}benzoic acid

5-cyano-2-{[(6-{[2-oxo-2-(4-oxo-3,4-dihydro-2H-1,3-benzoxazin-6-yl)ethyl]thio}pyridazin-3-yl)carbonyl]amino}benzoic acid

5-cyano-2-({[5-(2-methylphenyl)-1,2-benzisoxazol-3-yl]carbonyl}amino)benzoic acid

5-bromo-2-[({5-[(3R)-3-hydroxy-2-methoxy-4-(methoxymethoxy)butyl]pyrazin-2-yl}carbonyl)amino]benzoic acid

5-cyano-2-({[5-(pyrrolidin-1-ylsulfonyl)-i ,2-benzisoxazol-3-yl]carbonyl}amino)benzoic acid

5-cyano-2-[({5-[(dipropylamino)sulfonyl]-1,2-benzisoxazol-3-yl}carbonyl)amino]benzoic acid

2-[({5-[(5-chloro-2,3-dihydro-1H-indol-1-yl)sulfonyl]-1,2-benzisoxazol-3-yl}carbonyl)amino]-5-cyanobenzoic acid

5-cyano-2-{[(i-methyl-1H-indazol-3-yl)carbonyl]amino}benzoic acid

2-({[5-(anilinosulfonyl)-1,2-benzisoxazol-3-yl]carbonyl}amino)-5-bromobenzoic acid

5-bromo-2-({[5-(morpholin-4-ylsulfonyl)-1,2-benzisoxazol-3-yl]carbonyl}amino)benzoic acid

5-bromo-2-({[5-(hexylthio)pyridin-2-yl]carbonyl}amino)benzoic acid

5-cyano-2-[({1-methyl-6-[(thien-2-ylsulfonyl)amino]-1H-indol-2-yl}carbonyl)amino acid

5-cyano-2-{[(1-methyl-6-{[(methylamino)carbonyl]amino}-1H-indol-2-yl)carbonyl]amino}benzoic acid

5-cyano-2-{[(6-{[(isopropylamino)carbonyl]amino}-1-methyl-1H-indol-2-yl)carbonyl]acid

5-cyano-2-[({5-[(methylamino)sulfonyl]-1,2-benzisoxazol-3-yl}carbonyl)amino]benzoic acid compound with N,N-diethylamine (1:1)

2-{[(5-{[bis(2-hydroxyethyl)amino]sulfonyl}-1,2-benzisoxazol-3-yl)carbonyl]amino}

5-cyanobenzoic acid

2-({[6-(bis {[4-(acetylamino)phenyl]sulfonyl}amino)-1-methyl-1H-indol-2-yl]carbonyl}aam acid

2-[({6-[bis(thien-2-ylsulfonyl)amino]-1-methyl-1H-indol-2-yl}carbonyl)amino]-5-cyanoben acid

5-bromo-2-[({5-[(dimethylamino)sulfonyl]-1,2-benzisoxazol-3-yl}carbonyl)amino]benzoic acid

5-bromo-2-[({5-[(diethylamino)sulfonyl]-1,2-benzisoxazol-3-yl}carbonyl)amino]benzoic acid

2-[({5-[(benzylamino)sulfonyl]-1,2-benzisoxazol-3-yl}carbonyl)amino]-5-bromobenzoic acid

2-[({5-(acetylamino)-6-[4-(acetylamino)-2-(trifluoromethyl)phenyl]-1,2-benzisoxazol-3-yl}carbonyl)amino]benzoic acid

2-[({5-[3-(acetylamino)phenyl]-1,2-benzisoxazol-3-yl}carbonyl)amino]-5-cyanobenzoic acid or 5-Cyano-2-{[5-(3-acetamidophenyl)-benzisoxazole-3-carbonyl]amino}benzoic acid

5-cyano-2-[({5-[4-(methylsulfonyl)phenyl]-1,2-benzisoxazol-3-yl}carbonyl)amino]benzoic acid

5-bromo-2-({[5-(pyrrolidin-1-ylsulfonyl)-l ,2-benzisoxazol-3-yl]carbonyl}amino)benzoic acid

5-bromo-2-[({5-[(methylamino)sulfonyl]-1,2-benzisoxazol-3-yl}carbonyl)amino]benzoic acid

2-({[5-(aminosulfonyl)-1,2-benzisoxazol-3-yl]carbonyl}amino)-5-bromobenzoic acid

5-cyano-2-[({1-methyl-6-[(morpholin-4-ylcarbonyl)amino]-1H-indol-2-yl}carbonyl)amino]benzoic acid

5-cyano-2-[({5-[4-(morpholin-4-ylcarbonyl)phenyl]-1,2-benzisoxazol-3-yl}carbonyl)amino]acid

2-({[5-(2-acetylphenyl)-1,2-benzisoxazol-3-yl]carbonyl}amino)-5-cyanobenzoic acid

5-cyano-2-({[5-(2,5-dimethoxyphenyl)-1,2-benzisoxazol-3-yl]carbonyl}amino)benzoic acid

5-cyano-2-({[5-(2-phenoxyphenyl)isoxazol-3-yl]carbonyl}amino)benzoic acid

2-[({7-[3-(acetylamino)phenyl]-1-methyl-1H-indol-2-yl}carbonyl)amino]-5-cyanobenzoic acid

5-cyano-2-({[6-(2-methylphenyl)-1,2-benzisoxazol-3-yl]carbonyl}amino)benzoic acid

5-bromo-2-[({5-[(3S)-3,4-dihydroxybutyl]pyrazin-2-yl}carbonyl)amino]benzoic acid

5-cyano-2-({[6-(3,5-dimethylisoxazol-4-yl)-1,2-benzisoxazol-3-yl]carbonyl}amino)benzoic acid

2-[({5-[2-(acetylamino)phenyl]-1,2-benzisoxazol-3-yl}carbonyl)amio]-5-cyanobenzoic acid

5-cyano-2-[({7-[(ethylsulfonyl)amino]-1-methyl-1H-indol-2-yl}carbonyl)amino]benzoic acid

2-[({7-[bis(methylsulfonyl)amino]-1-methyl-1H-indol-2-yl}carbonyl)amino]-5-cyanobenzoic acid

5-cyano-2-{[(1-methyl-7-{[(1-methyl-1H-imidazol-4-yl)sulfonyl]amino}-1H-indol-2-yl)carbonyl]amino}benzoic acid

2-{[(7-{[(5-chloro-1,3-dimethyl-1H-pyrazol-4-yl)sulfonyl]amino}-1-methyl-1H-indol-2-yl)carbonyl]amino}-5-cyanobenzoic acid

2-[({7-[(butylsulfonyl)amino]-1-methyl-1H-indol-2-yl}carbonyl)amino]-5-cyanobenzoic acid

2-{[(7-{[(3-chloropropyl)sulfonyl]amino}-1-methyl-1H-indol-2-yl)carbonyl]amino}-5-cyanobenzoic acid

5-cyano-2-{[(7-{[(3,5-dimethylisoxazol-4-yl)sulfonyl]amino}-1-methyl-1H-indol-2-yl)carbonyl]amino}benzoic acid

2-{[(7-{bis[(5-bromo-6-chloropyridin-3-yl)sulfonyl]amino}-1-methyl-1H-indol-2-yl)carbonly]amino}benzoic acid

5-cyano-2-[({1-methyl-7-[(methylsulfonyl)amino]-1H-indol-2-yl}carbonyl)amino]benzoic acid

5-cyano-2-({[(6E)-6-(methoxyimino)-5,6-dihydro-4H-pyrrolo[3,2,1-ij]quinolin-2-yl]carbonyl}amino)benzoic acid

2-[({(6E)-6-[(benzyloxy)imino]-5,6-dihydro-4H-pyrrolo[3,2,1-ij]quinolin-2-yl}carbonyl)amino]-5-cyanobenzoic acid

5-cyano-2-({[(6E)-6-(phenoxyimino)-5,6-dihydro-4H-pyrrolo[3,2,1-ij]quinolin-2-yl]carbonyl}amino)benzoic acid

5-cyano-2-[({1-methyl-7-[(propylsulfonyl)amino]-1H-indol-2-yl}carbonyl)amino]benzoic acid

5-cyano-2-{[(7-{[(2,4-dimethyl-1,3-thiazol-5-yl)sulfonyl]amino}-1-methyl-1H-indol-2-yl)carbonyl]amino}benzoic acid

5-cyano-2-({[1-methyl-7-({[(methylsulfonyl)methyl]sulfonyl}amino)-1H-indol-2-yl]carbonyl}amino)benzoic acid

2-[({7-[3-(butylamino)-2-hydroxypropoxy]-1-methyl-1H-indol-2-yl}carbonyl)amino]

5-cyanobenzoic acid hydrochloride

5-cyano-2-[({7-[2-hydroxy-3-(2-phenoxyethoxy)propoxy]-1-methyl-1H-indol-2yl}carbonyl)amino]benzoic acid

5-cyano-2-{[(7-{2-hydroxy-3-[(3-methylbut-2-enyl)oxy]propoxy}-1-methyl-1H-indol-2-yl)carbonyl]amino}benzoic acid

5-cyano-2-[({7-[3-(2,3-dihydroxypropoxy)-2-hydroxypropoxy]-1-methyl-1H-indol-2-yl}carbonyl)amino]benzoic acid

2-[({7-[3-(but-3-ynyloxy)-2-hydroxypropoxy]-1-methyl-1H-indol-2-yl}carbonyl)amino]-acid

2-[({5-[acetyl(ethyl)amino]-1,2-benzisoxazol-3-yl}carbonyl)amino]-5-cyanobenzoic acid

5-cyano-2-[({5-[ethyl(methylsulfonyl)amino]-1,2-benzisoxazol-3-yl}carbonyl)amino]benzoic acid

5-cyano-2-{[(5-{[(dimethylamino)sulfonyl]amino}-1,2-benzisoxazol-3-yl)carbonyl]amino}benzoic acid

2-{[(6-bromo-1,2-benzisoxazol-3-yl)carbonyl]amino}-5-cyanobenzoic acid

2-[({6-[2-(acetylamino)phenyl]-1,2-benzisoxazol-3-yl}carbonyl)amino]-5-cyanobenzoic acid

2-[({6-[3-(acetylamino)phenyl]-1,2-benzisoxazol-3-yl}carbonyl)amino]-5-cyanobenzoic acid

5-cyano-2-({[(6E)-6-(ethoxyimino)-5,6-dihydro-4H-pyrrolo[3,2,1-ij]quinolin-2-yl]carbonyl}amino)benzoic acid

2-({[(6E)-6-(tert-butoxyimino)-5,6-dihydro-4H-pyrrolo[3,2,1-ij]quinolin-2-yl]carbonyl}amino)-5-cyanobenzoic acid

5-cyano-2-({[(6E)-6-(hydroxyimino)-5,6-dihydro-4H-pyrrolo[3,2,1-ij]quinolin-2-yl]carbonyl}amino)benzoic acid

5-bromo-2-{[(1,1-dioxido-1,2-benzisothiazol-3-yl)carbonyl]amino}benzoic acid

2-[(1,2-benzisothiazol-3-ylcarbonyl)amino]-5-bromobenzoic acid

5-cyano-2-({[7-(2-hydroxy-3-methoxypropoxy)-1-methyl-1H-indol-2-yl]carbonyl}amino)be acid

5-cyano-2-({[7-(3-ethoxy-2-hydroxypropoxy)-1-methyl-1H-indol-2-yl]carbonyl}amino)benzoic acid

2-({[7-(3-chloro-2-hydroxypropoxy)-1-methyl-1H-indol-2-yl]carbonyl}amino)-5-cyanobenzoic acid

5-cyano-2-[({1-methyl-7-[(pyridin-4-ylacetyl)amino]-1H-indol-2-yl}carbonyl)amni acid trifluoroacetate

5-cyano-2-[({5-[2-(hydroxymethyl)phenyl]-1,2-benzisoxazol-3-yl}carbonyl)amino]benzoic acid

5-cyano-2-[({6-[3-(hydroxymethyl)phenyl]-1,2-benzisoxazol-3-yl}carbonyl)amino]benzoic acid

5-cyano-2-[({5-[(methylsulfonyl)amino]-6-[4-[(methylsulfonyl)amino]-2(trifluoromethyl)phe

1,2-benzisoxazol-3-yl}carbonyl)amino]benzoic acid

5-bromo-2-[({6-[(dipropylamino)sulfonyl]-3-pyridinyl}carbonyl)amino]benzoic acid

5-bromo-2-{[(4-{[4-chloro(methyl)anilino]sulfonyl}-2-thienyl)carbonyl]amino}benzoic acid

5-bromo-2-{[(5-{[4-chloro(methyl)anilino]sulfonyl}-2-thienyl)carbonyl]amino}benzoic acid

5-bromo-2-[(5-{[4-chloro(methyl)anilino]sulfonyl}-2-furoyl)amino]benzoic acid

2-({[2-(benzylsulfanyl)-1H-imidazol-4-yl]carbonyl}amino)-5-bromobenzoic acid

5-bromo-2-{[(4-{[4-chloro(methyl)anilino]sulfonyl}-5-methyl-2thienyl)carbonyl]amino}benzoic acid

5-bromo-2-{[(4-{[4-chloro(methyl)anilino]sulfonyl}-3-methyl-2thienyl)carbonyl]amino}benzoic acid

5-bromo-2-{[(5-{[4-chloro(methyl)anilino]sulfonyl}-3-thienyl)carbonyl]amino}benzoic acid

5-bromo-2-[(2-thienylcarbonyl)amino]benzoic acid

2-{[2-(benzylsulfanyl)isonicotinoyl]amino}-5-bromobenzoic acid

5-bromo-2-{[(5-{[4-chloro(methyl)anilino]sulfonyl}-3pyridinyl)carbonyl]amino}benzoic acid

5-bromo-2-{[(5-{[4-chloro(methyl)anilino]sulfonyl}-1-oxido-3-pyridinyl)carbonyl]amino)-5-bromobenzoic acid

2-({[4-(benzylsulfanyl)-2-pyridinyl]carbonyl}amino)-5-bromobenzoic acid 2-({[6-(benzylsulfanyl)-2-pyridinyl]carbonyl}amino)-5-bromobenzoic acid.

The antibacterial agent may be incorporated into a pharmaceutical composition.

The pharmaceutical compositions of this invention may be prepared by combining the compounds of this invention with a solid or liquid pharrmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier can be at least one substance which may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the compounds of this invention dissolved in water and water-propylene glycol systems, optionally containing suitable conventional coloring agents, flavoring agents, stabilizers and thickening agents.

Preferably, the pharmaceutical composition is provided employing conventional techniques in unit dosage form containing effective or appropriate amounts of the active component, that is, the compound according to this invention.

The quantity of active component, that is the compound according to this invention, in the pharmaceutical composition and unit dosage form thereof may be varied or adjusted widely depending upon the particular application, the potency of the particular compound and the desired concentration. Generally, the quantity of active component will range between 0.5% to 90% by weight of the composition.

In therapeutic use for treating, or combatting, bacterial infections in warmblooded animals, the compounds or pharmaceutical compositions thereof will be administered orally, parenterally and/or topically at a dosage to obtain and maintain a concentration, that is, an amount, or blood-level of active component in the animal undergoing treatment which will be antibacterially effective. Generally, such antibacterially effective amount of dosage of active component will be in the range of about 0.1 to about 100, more preferably about 3.0 to about 50 mg/kg of body weight/day. It is to be understood that the dosages may vary depending upon the requirements of the patient, the severity of the bacterial infection being treated, and the particular compound being used. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., 2-4 four times per day.

The compounds according to this invention may be administered parenterally, i.e., by injection, for example, by intravenous injection or by other parenteral routes of administration. Pharmaceutical compositions for parenteral administration will generally contain a pharmaceutically acceptable amount of the compound or a soluble salt (acid addition salt or base salt) dissolved in a pharmaceutically acceptable liquid carrier such as, for example, water-for-injection and a buffer to provide a suitably buffered isotonic solution, for example, having a pH of about 3.5-6. Suitable buffering agents include, for example, trisodium orthophosphate, sodium bicarbonate, sodium citrate, N-methylglucamine, L(+)-lysine and L(+)-arginine to name but a few representative buffering agents. The compound of this invention generally will be dissolved in the carrier in an amount sufficient to provide a pharmaceutically acceptable injectable concentration in the range of about 1 mg/mL to about 400 mg/mL of solution. The resulting liquid pharmaceutical composition will be administered so as to obtain the above-mentioned antibacterially effective amount of dosage. The compounds according to this invention are advantageously administered orally in solid and liquid dosage forns.

As a topical treatment an effective amount of Formula I is admixed in a pharmaceutically acceptable gel or cream vehicle that can be applied to the patient's skin at the area of treatment. Preparation of such creams and gels is well known in the art and can include penetration enhancers.

The antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA.

In some embodiments, the antibacterial compounds are prodrugs of the compounds of formula I. The expression “prodrug” denotes a derivative of a known direct acting drug, which is transformed into the active drug by an enzymatic or chemical process. Prodrugs of the compounds of formula I are prepared by modifying functional groups present on the compound in such a way that the modifications are cleaved, either in routine manipulation or in vivo, to the parent compound. Prodrugs include, but are not limited to, compounds of structure (I) wherein hydroxy, amine or sulfhydryl groups are bonded to any group that, when administered to the animal, cleaves to form the free hydroxyl, amino or sulfhiydryl group, respectively. Representative examples of prodrugs include, but are not limited to, acetate, formate and benzoate derivatives of alcohol and amine functional groups. See Notari, R. E., “Theory and Practice of Prodrug Kinetics,” Methods in Enzymology, 112:309-323 (1985); Bodor, N., “Novel Approaches in Prodrug Design,” Drugs of the Future, 6(3): 165-182 (1981); and Bundgaard, H., “Design of Prodrugs: BioreversibleDerivatives for Various Functional Groups and Chemical Entities,” in Design of Prodrugs (H. Bundgaard, ed.), Elsevier, N.Y. (1985).

The antibacterial compounds of this invention may be synthesized by various methods known to those skilled in the art. Non-limiting examples of synthetic schemes for producing the antibacterial agents are described below.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example: 1 R₄ as an INDOLE or Derivative thereof:

Preparation of 7-(benzyloxy)-1H-indole-2-carboxylic acid

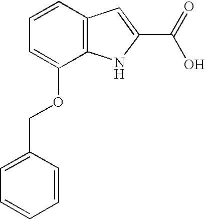

Ethyl 7-(benzyloxy)-1H-indole-2-carboxylate (645 mg, 2.18 mmol) and LiOH.H₂O (480 mg, 11.4 mmol) were combined in THF (10 mL) and H₂O (5 mL) and staken at 45° C. overnight. When the reaction was complete, the solution was diluted with MTBE, washed with 2 N HCl and brine, dried (MgSO₄), and concentrated in vacuo to afford 554 mg (95%) of the acid.

Analytical data

¹H NMR (300 MHz, DMSO-d₆) δ 12.84 (s, 1H), 11.84 (s, 1H), 7.65 (d, J=6.97 Hz, 2 H), 7.40 (t, J=6.97 Hz, 2 H), 7.33 (d, J=7.16 Hz, 1H), 7.22 (d, J=7.91 Hz, 1 H), 7.09 (d, J=2.26 Hz, 1H), 6.97 (t, J=7.72 Hz, 1H), 6.86 (d, J=7.15 Hz, 1H) 5.27 (s, 2 H).

Preparation of

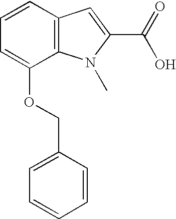

Preparation of

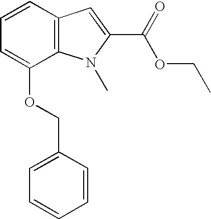

Ethyl 7-(benzyloxy)-1H-indole-2-carboxylate (10.22 g, 34.6 mmol) was dissolved in DMF (100 mL). NaH (60% dispersion, 2 g) was added and the reaction was stirred for 30 min at rt. Mel (25 mL) was added and solution stirred overnight. The reaction was diluted with MTBE, washed with H₂O x5, dried (MgSO₄), concentrated to afford 12.16 g (114%) Ethyl 7-(benzyloxy)-1-methyl-1H-indole-2-carboxylate as a yellow solid. The crude material was carried on as is.

The yellow solid product (7.5 g, 24.2 rnmoL) and LiOH.H₂O (9 g, 214 mmol) were combined in THF (100 mL) and H₂O (10 mL) and stirred at 45° C. overnight. When hydrolysis was complete, the solution was diluted with CH₂Cl₂, washed with 2 N HCl, dried (MgSO₄), and concentrated to afford 5.62 (83%) of the title compound as a white solid.

Analytical data

¹H NMR (300 MHz, DMSO-dd₆) δ 12.85 (br s, 1H), 7.54 (d, J=6.9 Hz, 2 H), 7.43 (t, J=7.38 Hz, 2 H), 7.36 (d, J=7.08 Hz, 1H), 7.22 (d, J=6.96 Hz, 1H), 7.17 (s, 1 H), 6.99 (t, J=7.83 Hz, 1H), 6.91 (d, J=6.93 Hz, 1H), 5.25 (s, 2 H), 4.30 (s, 3 H).

### Example 1.1: tert-Butyl-2-({[7-(benzyloxy)-1-methyl-1H-indol-2-yl]carbonyl}amino)-5-cyanobenzoate

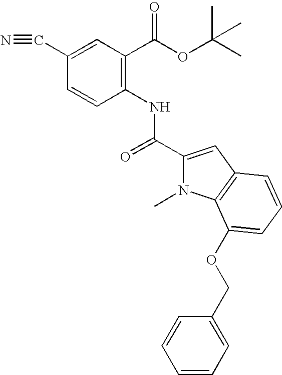

7-(Benzyloxy)-1-methyl-1H-indole-2-carboxylc acid (5.62 g, 20.0 mmol) and 4 drops of DMF were combined in CH₂CI₂ (150 mL). Oxalyl chloride (2.0 mL) was added and the suspension was stirred until clear, 4 hrs. The volatiles were removed in vacuo and the resulting acid chloride was placed under high-vac for 30 minutes to ensure removal of the oxalyl chloride. The acid chloride was re-dissolved in CH₂Cl₂ (70 mL), added to a solution of tert-butyl-2-amino-5-cyanobenzoate (4.15 g, 19.0 mrnol) in CH₂Cl₂ (70 mL) and pyridine (6 mL), and stirred at rt overnight. The solution was diluted with CH₂Cl₂, washed with 2 N HCl, dried (MgSO₄), concentrated, and triturated with MeOH to afford 7.25 g (75%) of the title compound as yellow solid.

Analytical data

¹H NMR (300 MHz, DMSO-d₆) δ 11.85 (s, 1H), 8.63 (d, J=8.79 Hz, 1H), 8.34 (d, J=2.01 Hz, 1H), 8.08 (dd, J=8.64, 2.07 Hz, 1H), 7.55 (d, J=6.96 Hz, 2 H), 7.44 (t, J=7.53 Hz, 2 H), 7.37 (d, J=7.10 Hz, 1H), 7.33 (d, J=7.35 Hz, 1H), 7.22 (s, 1 H), 7.05 (t, J=7.92 Hz, 1H), 6.95 (d, J=7.35 Hz, 1H), 5.28 (s, 2 H), 4.29 (s, 3 H), 1.59 (s, 9 H).

### Example 1.2: 5-Cyano-2-{[(7-hydroxy-1-methyl-1H-indol-2-yl)carbonyllamino}benzoic acid

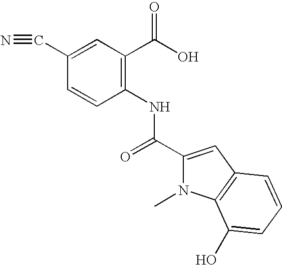

The compound from Example 1.1 (152 mg, 0.316 mmol) was dissolved in CH₂CI₂ (5 mL). TiCl₄ (1.0 M in CH₂Cl₂, 10 mL) was added slowly and the reaction was stirred for 20 min. The reaction was quenched with MeOH, concentrated, and triturated with MeOH for afford 63 mg (59%) of the title compound as a white solid.

Analytical data

¹H NMR (400 MHz, DMSO-d₆) δ 12.31 (s, 1H), 9.95 (s, 1H), 8.82 (d, J=6.63 Hz, 25 1 H), 8.41 (d, J=1.56 Hz, 1H), 8.08 (dd, J=6.60, 1.59 Hz, 1H), 7.15 (s, 1H), 7.12 (d, J=5.52 Hz, 1H), 6.90 (t, J=5.79 Hz, 1H), 6.67 (d, J=5.64 Hz, 1 H), 4.32 (s, 3 H).

### Example 1.3: 5-Cyano-2-({[1-methyl-7-(3-phenoxypropoxy)1H-indol-2-yl]carbonyl}amino)benzoic acid

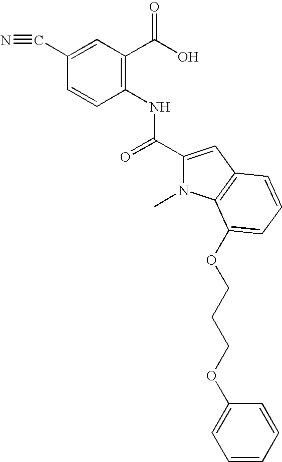

General method A: The compound of Example 1.1(500 mg, 1.28 mmol), PPh₃ (677 mg, 2.58 mmol), and 3-phenyloxy-1-propanol (388 mg, 2.55 mmol) were combined in THF (10 mL) and cooled in an ice bath. DIAD (570 pL, 2.89 mmol) was added over 1 min. The reaction was shaken overnight on an orbital shaking block at rt. The reaction was absorbed on SiO₂ and purified using silica gel chromatography (EtOAc/Hep: 1/19, 1/4) to afford 261 mg (39%) of the t-Bu ester, 36883-bdw-33. In some occasions a DIAD product co-eluted with the product, in which case the coelutants were triturated with MeOH to afford the clean desired ester. The ester (255 mg, 485 mmol) and LiOHeH₂O (262 mg, 624 mmol) were dissolved in THF (10 mL) and H₂O (1 mL) and shaken at 45° C. overnight. When the hydrolysis was complete, the reaction was diluted with CH₂Cl₂, washed with 2 N HCl, dried (MgSO₄), concentrated and triturated with MeOH to afford 73 mg (32%) of the title compound as a white solid.

Analytical data

¹H NMR (300 MHz, DMSO-d₆) δ 12.40 (s, 1H), 8.81 (d, J=8.80 Hz, 1H), 8.41 (d, J=2.06 Hz, 1H), 8.08 (dd, J=8.78, 2.11 Hz, 1H), 7.31-7.26 (m, 3 H), 7.18 (s, 1 H), 7.06-6.85 (m, 5 H), 4.31-4.29 (m, 5), 4.21 (t,J=6.17 Hz, 2 H), 2.36-2.29 (m, 2 H).

### Example 1.4: 2-({[7-(Benzyloxy)-1H-indol-2-yll carbonyl}amino)-5-cyanobenzoic acid

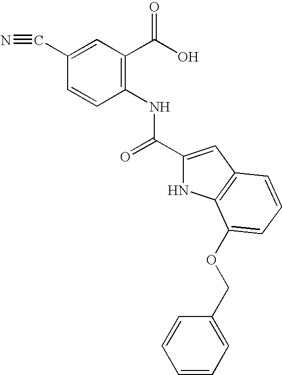

General procedure B: The acid, 7-(benzyloxy)-1H-indole-2-carboxylic acid (146 mg, 0.546 mmol) and 2 drops of DMF were combined in CH₂Cl₂ (10 mL). Oxalyl chloride (300 ,L) was added and the reaction was stirred for 4 hrs. Heptane was added and the volatiles were removed in vacuo. The resulting acid chloride was dissolved in CH₂Cl₂ (10 mL), added to a solution of tert-butyl 2-arino-5-cyanobenzoate (118 mg, 0.541 mmol) in CH₂Cl₂ (10 muL) and pyridine (1 mL), and the reaction was shaken overnight at rt. The solution was then diluted with MTBE, washed with 2 N HCl and brine, dried (MgSO₄), concentrated, and triturated with MeOH to afford 142 mg (56%) of the title compound as a white solid.

Analytical data

¹H NMR (400 MHz, DMSO-d₆) δ 12.48 (s, 1H), 12.10 (s, 1H), 8.84 (d, J=8.80 Hz, 1H), 8.42 (d, J=2.08 Hz, 1H), 8.10 (dd, J=8.76, 2.09 Hz, 1H), 7.65 (d, J=7.00, Hz, 2 H), 7.41 (t, J=7.64 Hz, 2 H), 7.34 (d, J=7.36 Hz, 1H), 7.30 (d, J=7.96 Hz, 1H), 7.17 (d, J=2.16 Hz, 1H), 7.02 (t, J=7.88 Hz, 1H), 6.90 (d, J=7.44 Hz, 1 H), 5.30 (s, 2 H).

### Example 1.5: 5-Cyano-2-({[7-(2,3-dihydro-1,4-benzodioxin-2-ylmethoxy)-1methyl-1H-indol-2-yl]carbonyl}amino)benzoic acid

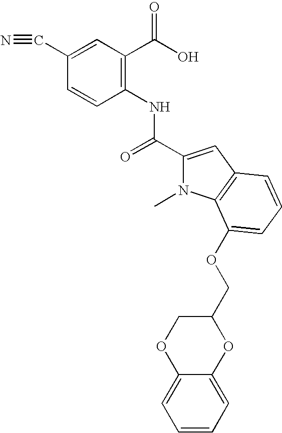

Prepared according to method A: The compound from examplel . (401 mg, 1.02 mmol) and 2,3-dihydro-1,4-benzodioxin-2-ylmethanol (350 mg, 2.11 nmmol) afforded 237 mg (42%) of the t-Bu ester. The ester (234 mg, 4.34 mmol) was hydrolyzed to afford 125 mg (56%) of a white solid.

Analytical data

¹H NMR (400 MHz, DMSO-d₆) δ 12.41 (s, 1H), 8.81 (d, J=8.88 Hz, 1H), 8.41 (d, J=2.08 Hz, 1H), 8.08 (dd, J=8.88, 2.04 Hz, 1H), 7.31 (d, J=8.08 Hz, 1H), 7.20 (s, 1H), 7.05 (t, J=7.88 Hz, 1H), 6.94-6.85 (m, 5 H), 4.74-4.68 (m, 1H), 4.52 (dd, J=11.60, 2.28 Hz, 1H), 4.44 (dd, J=10.60, 4.10 Hz, 1H), 4.37 (dd, J=10.36, 5.56 Hz, 1H), 4.30 (s, 3 H), 4.23 (dd, J=11.40, 7.04 Hz, 1H).

### Example 1.6: 5-Cyano-2-({[7-(cyclobutylmethoxy)-1-methyl-1H-indol-2-yllcarbonyl}amino)benzoic acid

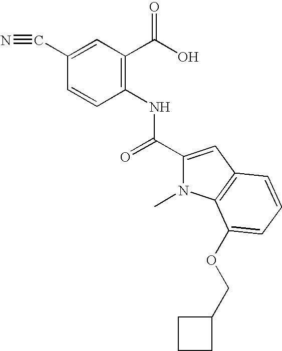

Prepared according to method A: The compound of exaipole 1.1 (384 mg, 0.981 mmol) and cyclobutane methanol (200 μL, 2.12 mmol) afforded 299 mg (66%) of the t-Bu ester. The ester (242 mg, 0.527 nmmol) was hydrolyzed to afford 171 mg (80%) of a white solid following recrystalization with MeOH/CH₂Cl₂.

Analytical data

¹H NMR (400 MHz, DMSO-d₆) δ 12.40 (s, 1H), 8.81 (d, J=8.80 Hz, 1H), 8.41 (d, J=2.08 Hz, 1H), 8.08 (dd, J=8.84, 2.12 Hz, 1H), 7.26 (d, J=7.48 Hz, 1H), 7.19 (s, 1H), 7.02 (t, J=7.80 Hz, 1H), 6.82 (d, J=7.36 Hz, 1H), 4.31 (s, 3 H), 4.08 (d, J=6.52Hz, 2 H), 2.90-2.83 (m, 1H), 2.14-2.11 (m, 2 H), 1.95-1.89 (m, 4 H).

### Example 1.7: 5-Cyano-2-[({7-[(2,2-dimethyl-1,3-dioxolan-4-yl)methoxy]-1-methyl-1H-indol-2-yl}carbonyl)aminolbenzoic acid

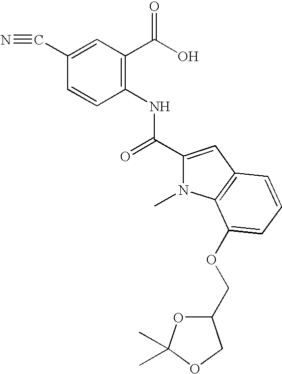

Prepared according to method A: The compound of example 1.1 (709 mg, 1.81 mmol) and (R)-(−)-2,2-dimethyl-1,3-dioxolane-4-methanol (450 mg, 3.62 mmol) afforded 467 mg (51 %) of the t-Bu ester. The ester (202 mg, 400 mmol) was hydrolyzed to 5 afford 127 mg (71%) of a white solid following recrystalization with MeOH/CH₂Cl₂.

Analytical data

¹H NMR (400 MHz, DMSO-d₆) δ 12.40 (s, 1H), 8.81 (d, J=8.80 Hz, 1H), 8.41 (d, J=2.04 Hz, 1H), 8.09 (dd, J=8.80, 2.12 Hz, 1H), 7.29 (d, J=7.52 Hz, 1H), 7.20 (s, 1H), 7.03 (t, J=7.88 Hz, 1H), 6.86 (d, J=7.40 Hz, 1H), 4.54 (quintet, J=5.80 Hz, 1H),4.31 (s, 3 H), 4.19-4.13 (m, 3 H), 3.89 (dd, J=8.36, 6.04 Hz, 1H), 1.39 (s, 3 H), 1.33 (s, 3 H).

### Example 1.8: 5-Cyano-2-({17-(2,3-dihydroxypropoxy)-1-methyl-1H-indol-2-yl]carbonyl}amino)benzoic acid

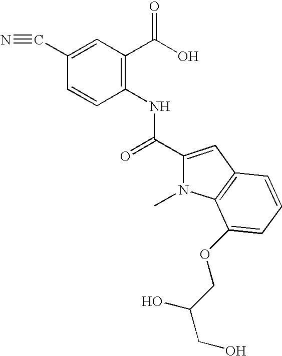

The compound of Examplel.7 (150 mg, 0.334 mmol) was dissolved in CH₂Cl₂ (4 ml), TFA (3 mL), and H₂O (3 drops) and shaken at rt for 20 min. Heptane was added to the solution and the volatiles were removed in vacuo. The crude product was purified by recrystalization from MeOH/CH₂Cl₂ to afford 74 mg (54%) of a white solid.

Analytical data

¹H NMR (400 MHz, DMSO-d₆) δ 12.40 (s, 1H), 8.82 (d, J=8.80 Hz, 1H), 7.41 (d, J=2.08 Hz, 1H), 8.09 (dd, J=8.72, 2.08 Hz, 1H), 7.26 (d, J=7.96 Hz, 1H), 7.19 (s, 1H), 7.03 (t, J=7.88 Hz, 1H), 6.82 (d, J=7.64 Hz, 1H), 4.33 (s, 3 H), 4.15 (dd, J=9.76, 4.16 Hz, 1H), 4.03 (dd, J=9.72, 5.60 Hz, 1H), 3.91 (quintet, J=5.40 Hz, 1H), 3.53 (d,J=5.76Hz,2H).

### Example 1.9: 5-Cyano-2-({17-(cyclobutyloxy)-1-methyl-1H-indol-2-yl]carbonyl}amino)benzoic acid

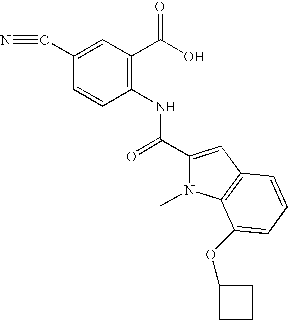

Prepared according to method A: The compound of example 1.1 (402 mg, 1.03 mmol) cyclobutyl alcohol (161 AL, 2.06 mmol) afforded 240 mg (52%) of the t-Bu ester. The ester (240 mg, 0.539 mmol) was hydrolyzed to afford 154 mg (73%) of a white solid following recrystalization with MeOH/CH₂Cl₂.

Analytical data

¹H NMR (400 MHz, DMSO-d₆) δ 12.35 (s, 1H), 8.81 (d, J=8.80 Hz, 1H), 8.41 (d, J=2.12 Hz, 1H), 8.08 (dd, J=8.80, 2.12 Hz, 1H), 7.25 (d, J=7.44 Hz, 1H), 7.18 (s, 1H), 7.00 (t, J=7.88 Hz, 1H), 6.66 (d, J=7.44 Hz, 1H), 4.84 (quintet, J=7.08 Hz, 1H), 4.32 (s, 3 H), 2.51-2.48 (m, 2 H), 2.19-2.12 (m, 2 H), 1.90-1.82 (m, 1H), 1.74-1.68 (m, 1 H).

### Example 1.10: 5-Cyano-2-({[7-(2-methoxy-1-methylethoxy)-1-methyl-1H-indol-2-yl] carbonyl}amino)benzoic acid

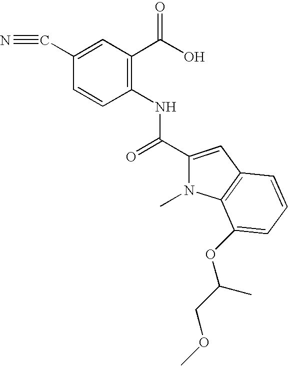

Prepared according to method A: The compound of example 1.1 (428 mg, 1.09 mmol) and 2-hydroxy-1-methoxypropane (220 μL, 2.25 mmol) afforded 249 mg (49%) of the t-Bu ester. The ester (246 mg, 0.531 mmol) was hydrolyzed to afford 138 mg (63%) of a white solid following recrystalization with MeOHWCH₂Ci₂.

Analytical data

¹H NMR (300 MHz, DMSO-d₆) δ 12.40 (s, 1H), 8.82 (d, J=8.82 Hz, 1H), 8.41 (d, J=2.07 Hz, 1H), 8.08 (dd, J=8.79, 2.10 Hz, 1H), 7.26 (d, J=7.50 Hz, 1H), 7.18 (s, 1H), 7.03 (t, J=7.86 Hz, 1H), 6.91 (d, J=7.56 Hz, 1H), 4.76 (sextet, J=4.55 Hz, 1H), 4.29 (s, 3 H), 3.63-3.54 (m, 2 H), 3.32 (s, 3 H), 1.33 (d, J=6.21 Hz, 3

### Example 1.11: 5-Cyano-2-{[(7-isopropoxy-1-methyl-1H-indol-2-yl)carbonyl]amino}benzoic acid

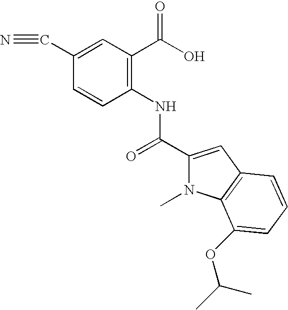

Prepared according to method A: The compound of examplel .1 (409 mg, 1.04 mmol) and isopropanol (300 gL) afforded 291 mg (65%) of the t-Bu ester. The ester (288mg, 0.664 mmol) was hydrolyzed to afford 158 mg (63%) of a white solid following recrystalization with MeOH/CH₂Cl₂.

Analytical data

¹H NMR (300 MHz, DMSO-d₆) δ 12.35 (s, 1H), 8.81 (d, J=8.82 Hz, 1H), 8.40 (d, J=2.04 Hz, 1H), 8.07 (dd, J=8.82, 2.10 Hz, 1H), 7.24 (d, J=7.92 Hz, 1H), 7.17 (s, 1H), 7.02 (t, J=7.89 Hz, 1H), 6.86 (d, J=7.62 Hz, 1H), 4.76 (septet, J=6.06 Hz, 1H), 4.29 (s, 3 H), 1.37 (d, J=6.00 Hz, 6 H).

### Example 1.12: 2-({[7-(Benzyloxy)-1-methyl-1H-indol-2-yll carbonyl}amino)-5-cyanobenzoic acid

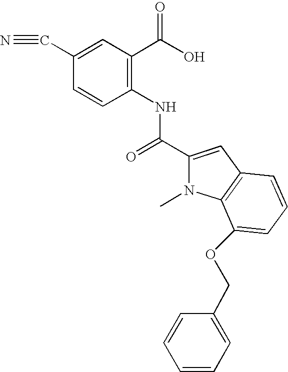

Prepared according to method B: 7-(benzyloxy)-1-methyl-1H-indole-2-carboxylic acid (7.80 g, 27.7 mmol) and methyl

5-cyanoanthranilate (4.80 g, 27.2 mmol) afforded 8.37 g (70%) of the methyl ester. The ester (398 mg, 0.906 mmol) was hydrolyzed to afford 327 mg (85%) of a white solid.

Analytical data

¹H NMR (400 MHz, DMSO-d₆) δ 12.39 (s, 1H), 8.80 (d, J=8.71 Hz, 1H), 8.40 (d, J=2.08 Hz, 1H), 8.07 (dd, J=8.71, 2.08 Hz, 1H), 7.56 (d, J=7.05 Hz, 2 H), 7.44 (t, J=7.26 Hz, 2 H), 7.36 (t, J=7.25 Hz, 1H), 7.29 (d, J=7.67 Hz, 1H), 7.19 (s, 1 H), 7.04 (t, J=7.67 Hz, 1H), 6.94 (d, J=7.47 Hz, 1H), 5.27 (s, 2 H), 4.30 (s, 3 H).

### Example 1.13: 5-Cyano-2-({[7-(cyclopropylmethoxy)-1-methyl-1H-indol-2-yl]carbonyl}amino)benzoic acid

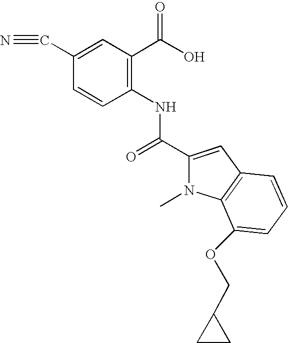

Prepared according to method A: The compound of example 1.1 (399 mg, 1.02 nmiol) and cyclopropyl alcohol (180 lL, 2.22 mmol) afforded 249 mg (55%) of the t-Bu ester. The ester (246 mg, 0.552 mmol) was hydrolyzed to afford 154 mg (70%) of a white solid following recrystalization with MeOH/CH₂Cl₂.

Analytical data

¹H NMR (300 MHz, DMSO-d₆) δ 12.39 (s, 1H), 8.82 (d, J=8.86 Hz, 1H), 7.41 (d, J=2.07 Hz, 1H), 8.08 (dd, J=8.67, 2.07 Hz, 1H), 7.26 (d, J=7.54 Hz, 1H), 7.18 (s, 1H), 7.01 (t, J=6.79 Hz, 1H), 6.80 (d, J=7.35 Hz, 1H), 4.35 (s, 3 H), 3.98 (d, J=6.79 Hz, 2 H), 1.41-1.27 (m, 1H), 0.65-0.59 (m, 2 H), 0.43-0.37 (m, 2 H).

### Example 1.14: 5-Cyano-2-({[7-(cyclohexylmethoxy)-1-methyl-1H-indol-2-yl]carbonyl}amino)benzoic acid

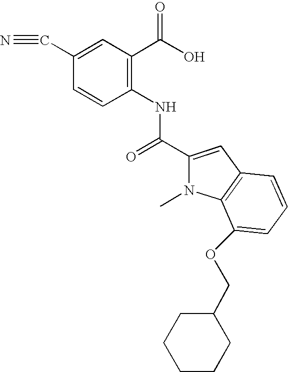

Prepared according to method A: The compound of examplel 1.1 (413 mg, 1. 06 mmol) and cyclohexane methanol (I150 jiL) afforded 261 mg (52%) of the t-Bu ester. The ester (257 mg, 0.543 mimol) was hydrolyzed to afford 173 mig (75%) of a white solid following recrystalization with MeOH/CH₂Cl₂.

Analytical data

¹H NMR (400 MHz, DMSO-d6) 5 12.3 9 (s, 1H), 8.81 (d, J=8.92 Hz, 1H), 8.41 (d, J=2.07 Hz, 1H), 8.08 (dd, J=8.71, 2.07 Hz, 1H), 7.26 (d, J=7.67 Hz, 1H), 7.18 (s, 1H), 7.02 (t, J=7.88 Hz, 1H), 6.83 (d, J=7.67 Hz, I1H), 4.30 (s, 3H), 3.98 (d, J=6.64, 6.42 Hz, 2 H), 2.06-1.95 (m, 1H), 1.89-1.77 (m, 4 H), 1.64-1.50 (m, 4H), 1.22-1.15 (m, 2H).

### Example 1.15: 5-Cyano-2-(J[7-(2-methoxyethoxy)-1-methyl-1H-indol-2-yl]carbonyl}amino)benzoic acid

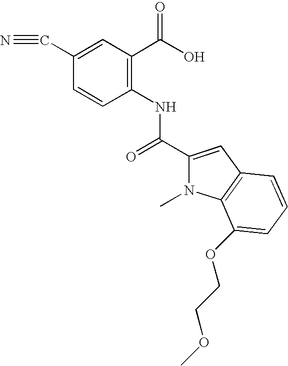

Prepared according to method A: The compound of example 1.1 (411 mg, 1.05 mmol) and 2-methoxy ethanol (170 lL, 2.16 mmol) afforded 323 mg (68%) of the t-Bu ester. The ester (319 mg, 0.638 nmuol) was hydrolyzed to afford 228 mg (81%) of a white solid following recrystalization with MeOH/CH₂Cl₂.

Analytical data

¹H NMR (400 MHz, DMSO-d₆) δ 12.37 (s, 1H), 8.81 (d, J=8.71 Hz, 1H), 8.40 (d, J=2.28 Hz, 1H), 8.07 (dd, J=8.91, 2.07 Hz, 1H), 7.27 (d, J=8.08 Hz, 1H), 7.18 (s, 1H), 7.03 (t, J=7.88 Hz, 1H), 6.84 (d, J=7.67 Hz, 1H), 4.31 (s, 3 H), 4.26-4.23 (m, 2 H), 3.79-3.76 (m, 2 H)3.36 (s, 3 H).

### Example 1.16: 5-Cyano-2-({l -methyl-7-(tetrahydro-2H-pyran-2-ylmethoxy)1H-indol-2-yl]carbonyl}acid

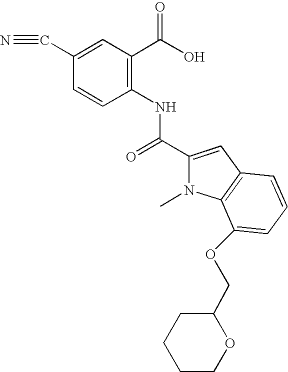

Prepared according to method A: The compound of examplel .1 (410 mg, 1.05 mmol) and 2-methanol tetrahydopyran (250 liL) afforded the t-Bu ester. The ester was hydrolyzed to afford 180 mg (40% 2-steps) of a white solid following recrystalization with MeOH/CH₂Cl₂.

Analytical data

¹H NMR (400 MHz, DMSO-d₆) δ 12.37 (s, 1H), 8.80 (d, J=8.59 Hz, 1H), 8.39 (d, J=2.53 Hz, 1H), 8.06 (dd, J=9.10, 2.02 Hz, 1H), 7.26 (d, J=7.57 Hz, 1H), 7.17 (s, 1H), 7.01 (t, J=8.09 Hz, 1H), 6.81 (d, J=7.58 Hz, 1H), 4.30 (s, 3 H), 4.06 (d, J=5.05 Hz, 2 H), 3.96-3.92 (m, 1H), 3.75-3.71 (mn, 1H), 3.47-3.40 (m, 1H), 1.87-1.84 (m, 1H), 1.74-1.70 (m, 1H), 1.59-1.40 (m, 4 H).

### Example 1.17: 5-Cyano-2-({[1-methyl-7-(oxiran-2-ylmethoxy)-1H-indol-2-yl]carbonyl}amino)benzoic acid

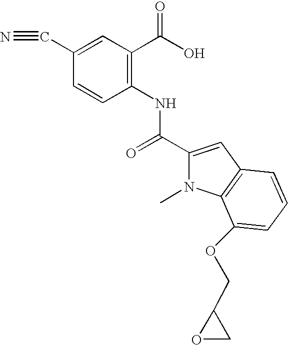

Prepared according to method A: The compound of example 1.1 (406.mg, 1.04 mrol) and glycidol (170 μL) afforded 52 mg (11 %) the t-Bu ester which was hydrolyzed to afford the title compound.

Analytical data

¹H NMR (400 MHz, DMS0-d₆) 5 11.79 (s, 1H), 8.62 (d, J=8.71 Hz, 1H), 8.34 (d, J=2.08 Hz, 1H), 8.09 (dd, J=8.71, 2.08 Hz, 1H), 7.33 (d, J=7.47 Hz, 1H), 7.22 (s, 1H), 7.04 (t, J=7.88 Hz, 1H), 6.86 (d, J=7.88 Hz, 1H), 4.49 (dd, J=11.20, 2.49 Hz, 1H), 4.30 (s, 3 H), 4.02 (dd, J=11.20, 6.22 Hz, 1H), 3.49-3.44 (m, 1H), 2.79 (dd, J=4.98, 4.35 Hz, 1H), 2.79 (dd, J=4.97, 2.69 Hz, 1H), 1.58 (s, 9 H).

### Example 1.18: 5-Cyano-2-({[7-(2-hydroxy-3-isopropoxypropoxy)-1-methyl-1H-indol-2-yl]carbonyl}amino)benzoic acid

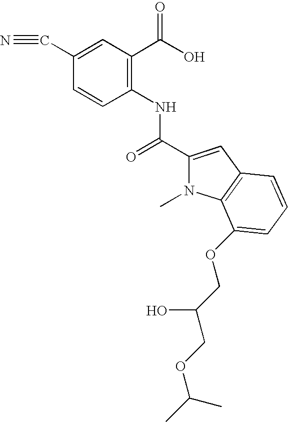

The epoxide, 5-Cyano-2-({I[1-methyl-7-(oxiran-2-ylmethoxy)-1H-indol-2-yl]carbonyl}amino)benzoic acid, (50 mg, 0.112 mmol), isopropanol (2 mL), and Yb(OTf)₃ (52 mg), and CH₂Cl₂ (2 mL) were combined and shaken at 45° C. overnight. The reaction was diluted with CH₂Cl₂, washed with H₂0, dried (MgSO₄), concentrated, and triturated with MeOH to afford 20 mg (36 %) of the t-Bu ester, 36883-bdw-108. The ester (20 mg, 0.0399 nrmol) was dissolved in THF (10 mL) and H₂O (1 mL) with LiOH (50 mg, 1.19 nimol) and shaken at 45° C. overnight. The acid was diluted with CH₂Cl₂, washed with 2 N HCl, dried (MgSO₄), concentrated, and triturated with MeOH to afford 11 mg (61%) of a white solid.

Analytical data

¹H NMR (400 MHz, DMSO-d₆) δ 12.43 (s, 1H), 8.82 (d, J=8.92 Hz, 1H), 8.41 (d, J=2.08 Hz, 1H), 8.09 (dd, J=8.91, 2.07 Hz, 1H), 7.27 (d, J=7.47 Hz, 1H), 7.19 (s, 1H), 7.03 (t, J=7.88 Hz, 1H), 6.82 (d, J=7.47 Hz, 1H), 4.33 (s, 3 H), 4.12 (dd, J=9.53, 3.94 Hz, 1H), 4.07-3.99 (m, 2 H), 3.59 (quintet, J=6.22 Hz, 1H), 3.54-3.46 (m, 2 H), 1.09 (dd, J=6.02, 3.11 Hz, 6 H).

### Example 1.19: 5-Cyano-2-({[7-(2-furyhnethoxy)-1-methyl-1H-indol-2-yljcarbonyl}amino)benzoic acid

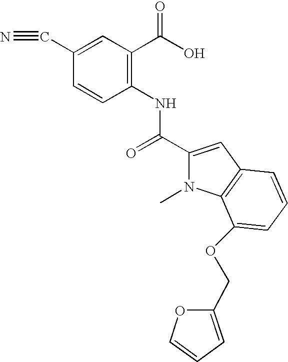

Prepared according to method A: The compound of example 1.1 (437 mg, 1.16 mmol) and 2-methanol furan (200 ,L, 2.31 mmol) afforded 75 mg (14%) of the t-Bu ester. The ester (72 mg, 0.157 mmol) was hydrolyzed to afford 19 mg (28%) of a white solid.

Analytical data

¹H NMR (400 MHz, DMSO-d₆) δ 12.35 (s, 1H), 8.80 (d, J=8.80 Hz, 1H), 8.41 (d, J=2.04 Hz, 1H), 8.08 (dd, J=8.80, 2.08 Hz, 1H), 7.74 (s, 1H), 7.32 (dd, J=7.00, 1.84 Hz, 1H), 7.19 (s, 1H), 7.08-7.04 (m, 2 H), 6.65 (d, J=3.16 Hz, 1H), 6.50 (dd, J=3.12,1.88 Hz, 1H), 5.23 (s, 2 H), 4.23 (s, 3 H).

### Example 1.20: 5-Cyano-2-({ll-methyl-7-(pentyloxy)-1H-indol-2-yl]carbonyl}amino)benzoic acid

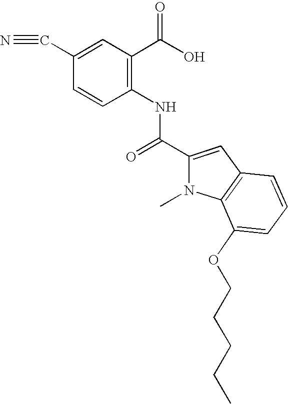

Prepared according to method A: The compound of example 1.1 (215 mg, 0.549 mmol) and 1-pentanol (120 μL, 1.11 mmol) afforded 58 mg (23%) of the t-Bu ester. The ester (55 mg, 0.119 mmol) was hydrolyzed to afford 7.0 mg (14%) of a white solid.

### Example 1.21-1.122

The following compounds were produced by making non-critical variations to the methods described above.

| | |
|---|---|
| 1.21 | 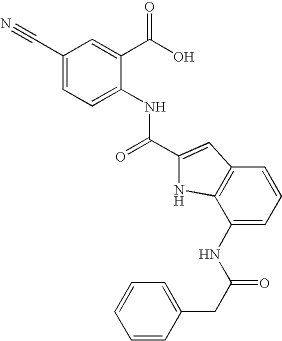 |
| 1.23 | 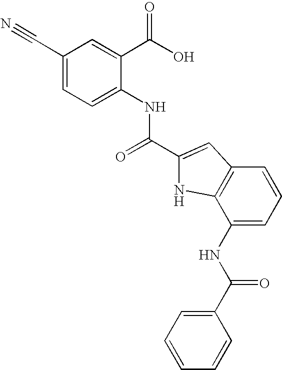 |
| 1.24 | 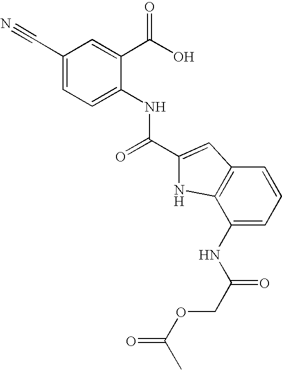 |
| 1.25 | 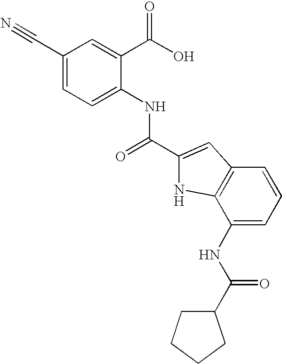 |
| 1.26 | 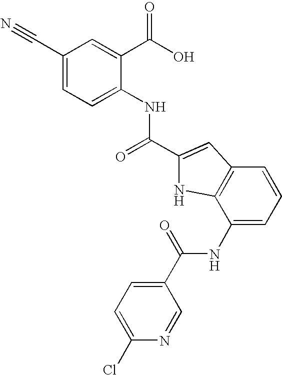 |
| 1.27 | 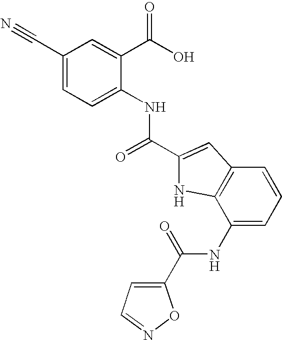 |
| 1.28 | 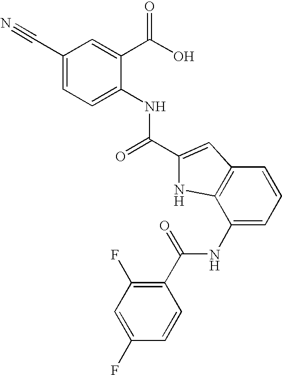 |
| 1.29 | 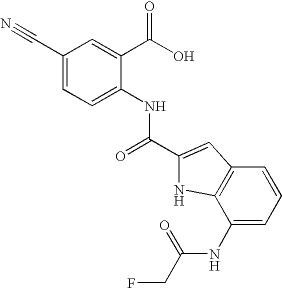 |
| 1.30 | 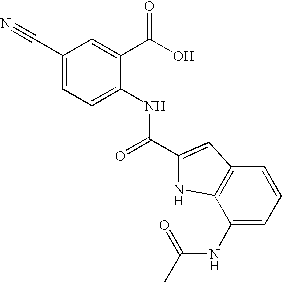 |
| 1.31 | 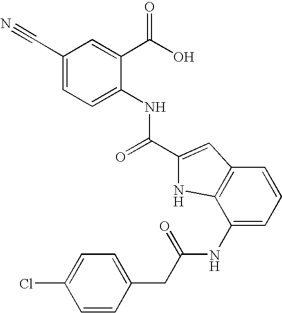 |
| 1.32 | 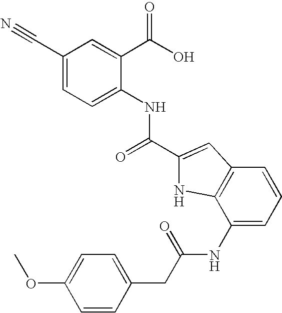 |
| 1.33 | 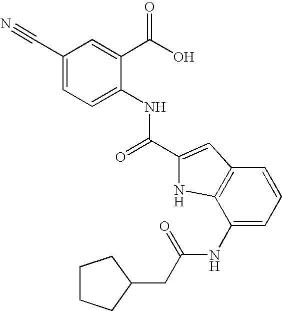 |
| 1.34 | 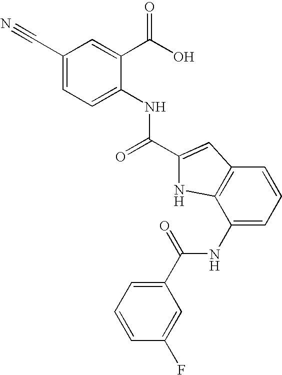 |
| 1.35 | 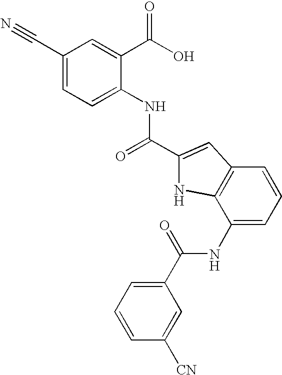 |
| 1.36 | 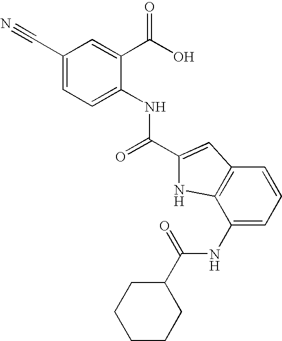 |
| 1.37 | 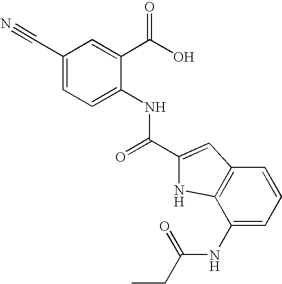 |
| 1.38 | 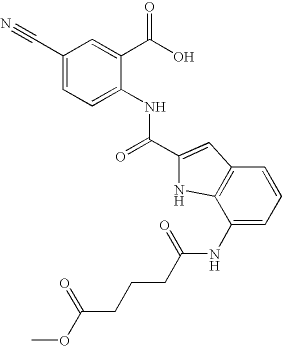 |
| 1.39 | 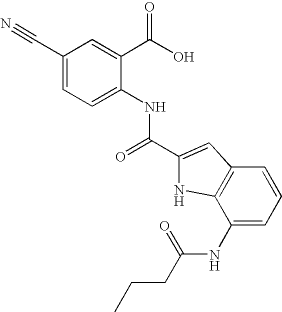 |
| 1.40 | 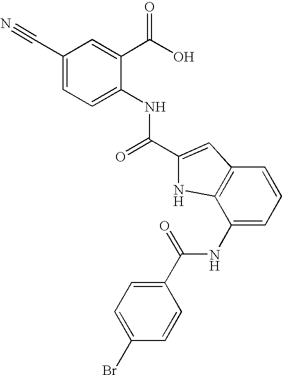 |
| 1.41 | 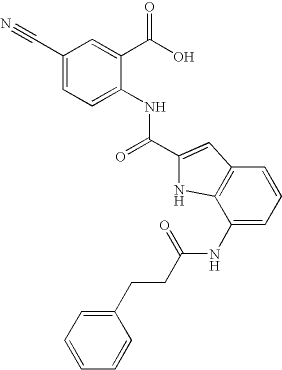 |
| 1.43 | 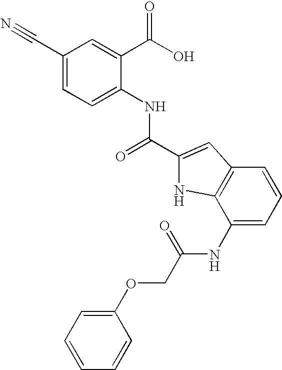 |
| 1.44 | 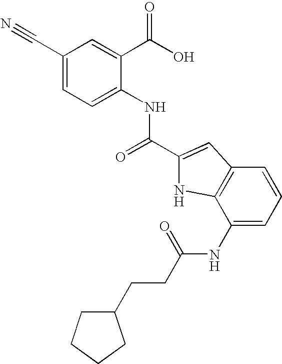 |
| 1.45 | 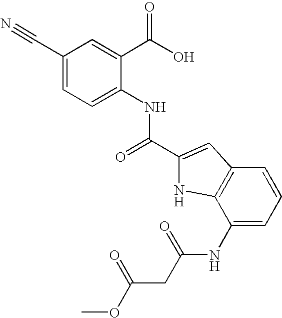 |
| 1.46 | 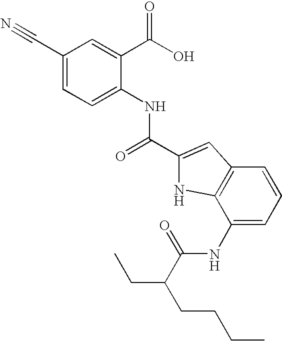 |
| 1.47 | 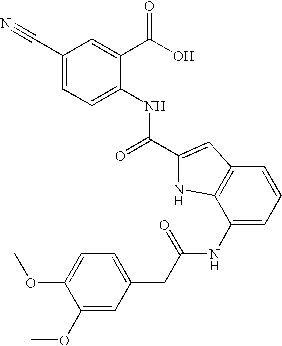 |
| 1.48 | 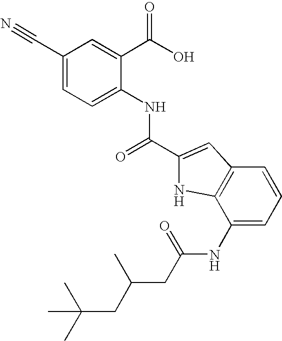 |
| 1.49 | 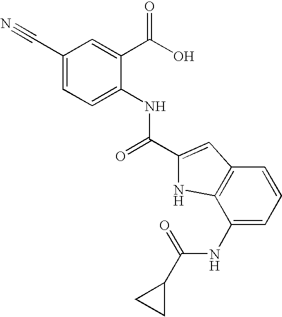 |
| 1.50 | 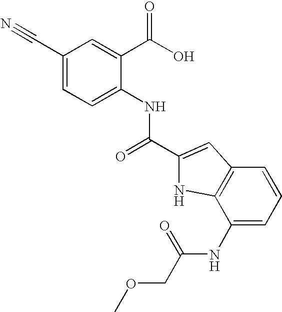 |
| 1.51 | 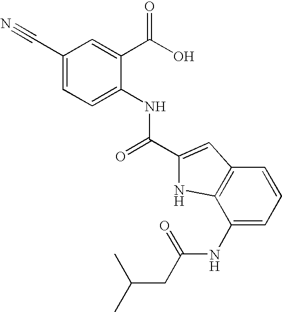 |
| 1.52 | 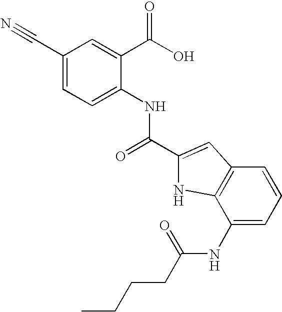 |
| 1.54 | 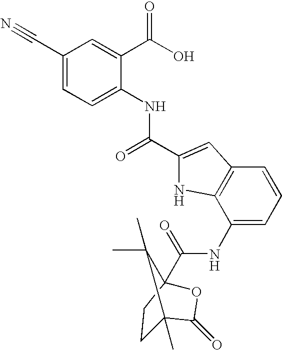 |
| 1.55 | 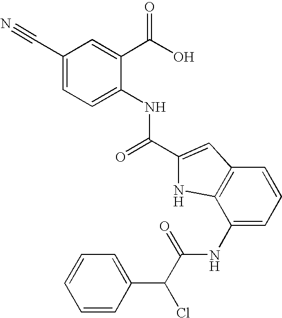 |
| 1.56 | 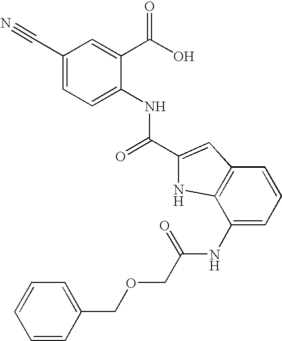 |
| 1.57 | 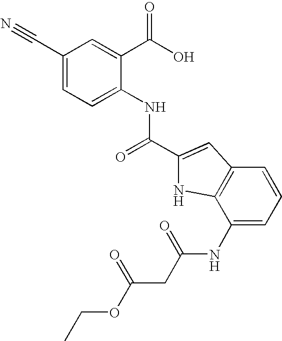 |
| 1.58 | 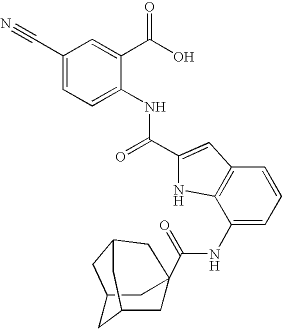 |
| 1.59 | 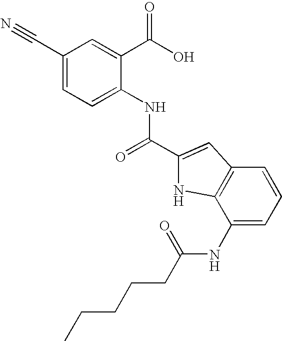 |
| 1.61 | 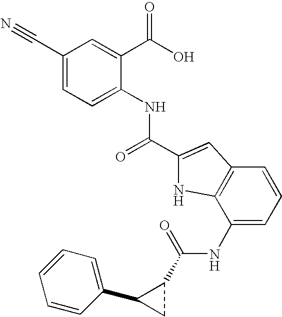 |
| 1.62 | 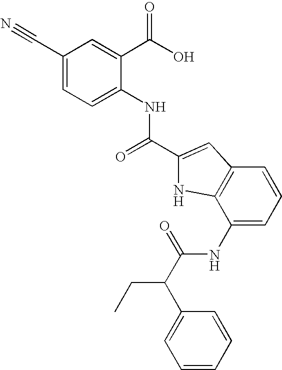 |
| 1.63 | 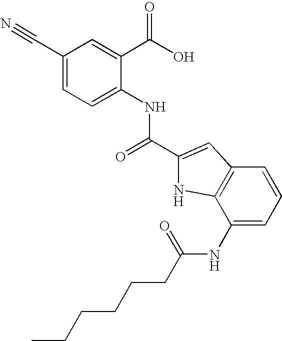 |
| 1.64 | 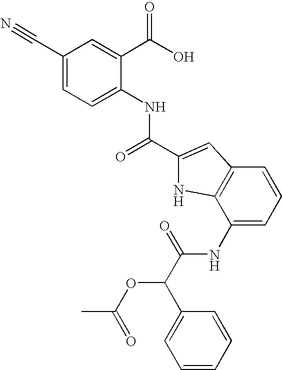 |
| 1.65 | 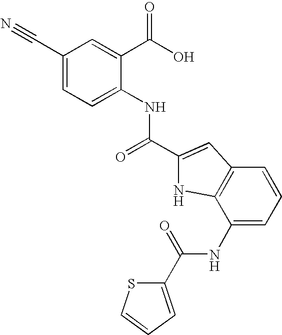 |
| 1.66 | 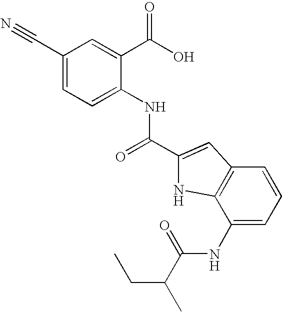 |
| 1.67 | 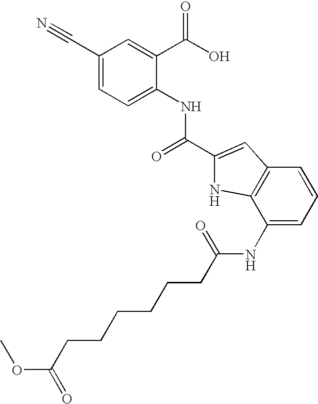 |
| 1.68 | 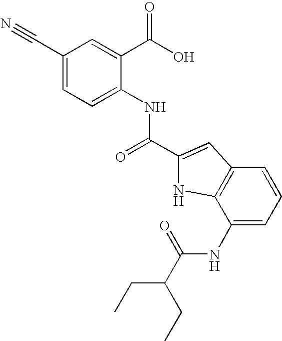 |
| 1.69 | 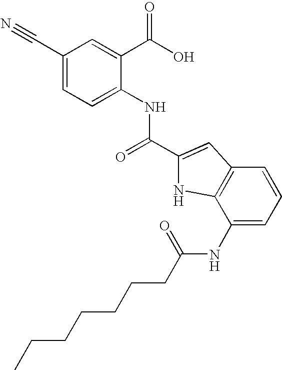 |
| 1.70 | 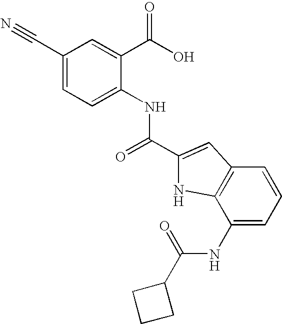 |
| 1.71 | 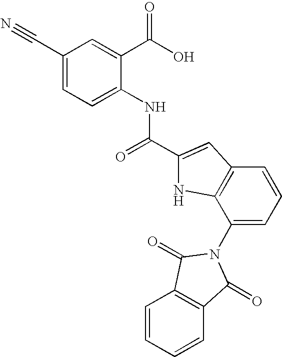 |
| 1.72 | 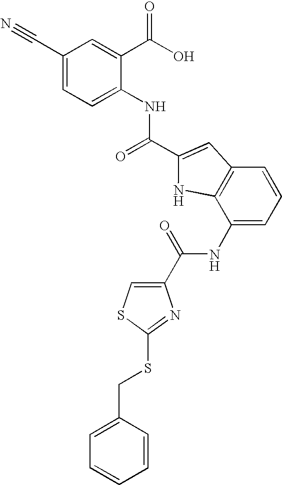 |
| 1.73 | 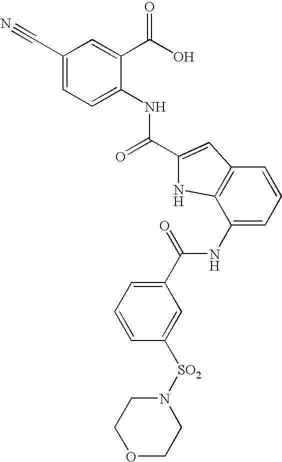 |
| 1.74 | 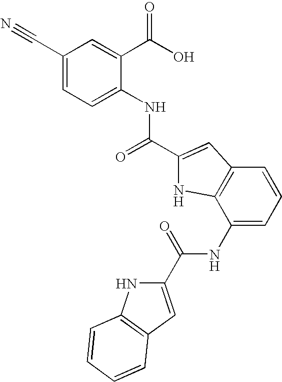 |
| 1.75 | 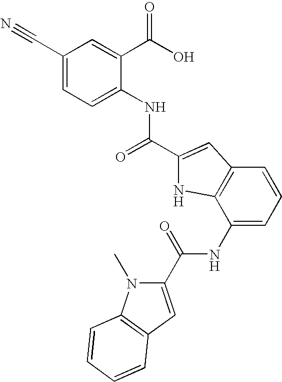 |
| 1.76 | 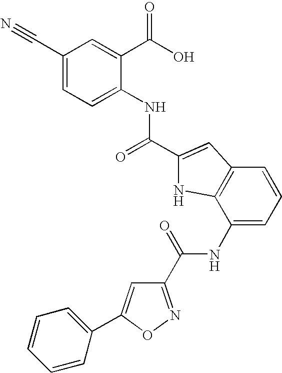 |
| 1.77 | 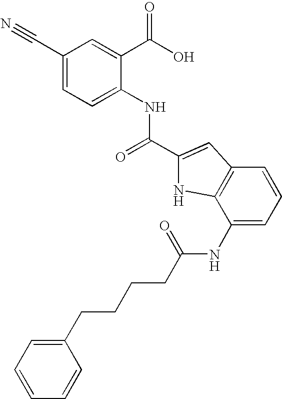 |
| 1.78 | 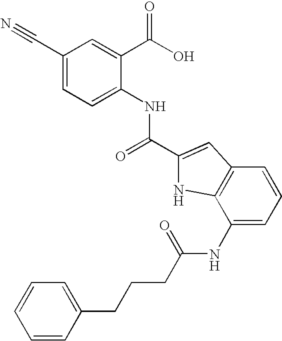 |
| 1.79 | 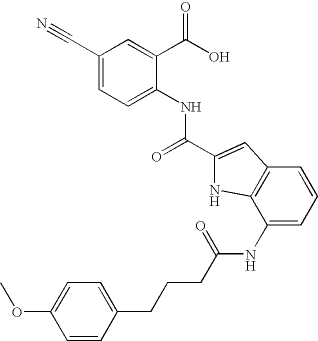 |
| 1.80 | 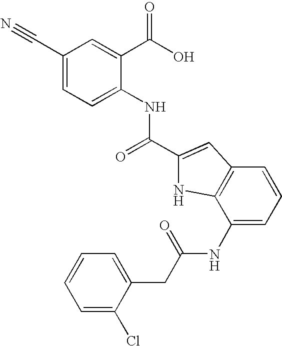 |
| 1.81 | 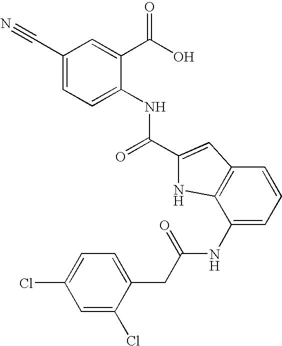 |
| 1.82 | 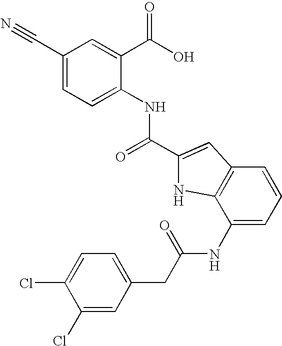 |
| 1.83 | 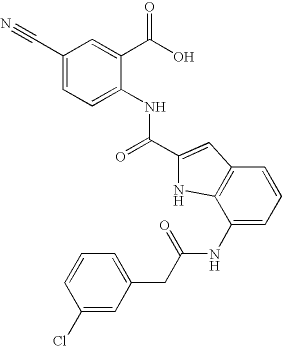 |
| 1.84 | 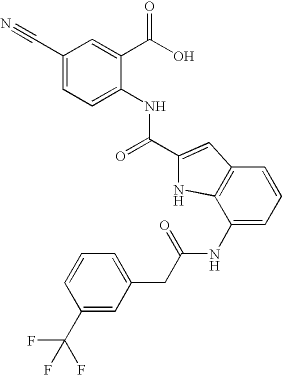 |
| 1.85 | 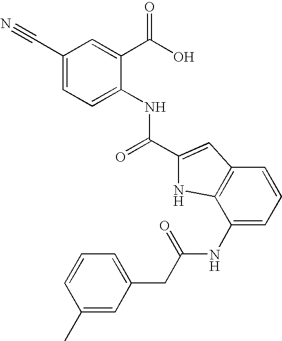 |
| 1.86 | 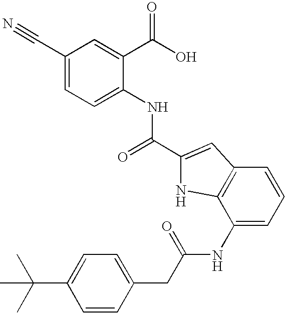 |
| 1.87 | 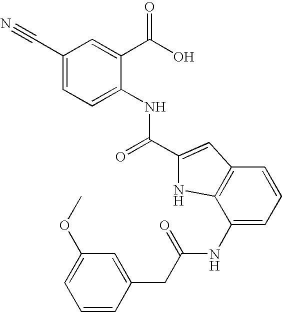 |
| 1.88 | 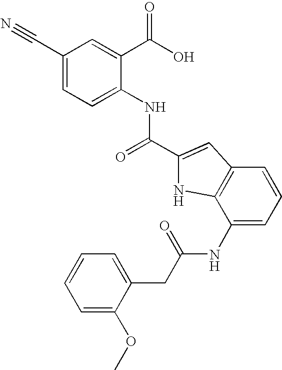 |
| 1.89 | 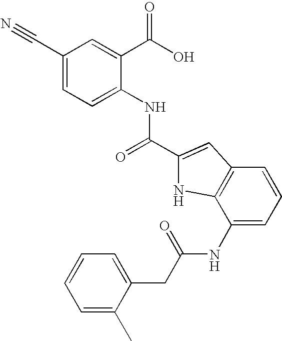 |
| 1.90 | 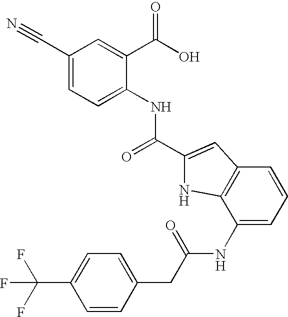 |
| 1.91 | 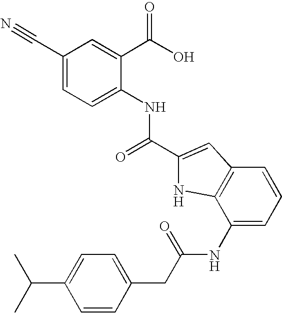 |
| 1.92 | 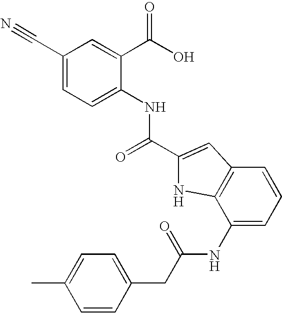 |
| 1.93 | 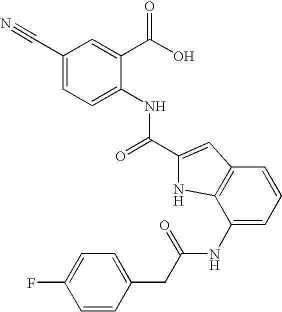 |
| 1.94 | 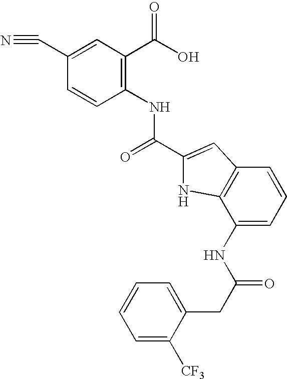 |
| 1.95 | 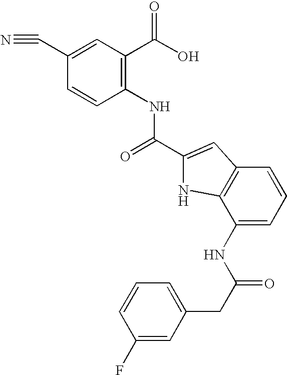 |
| 1.96 | 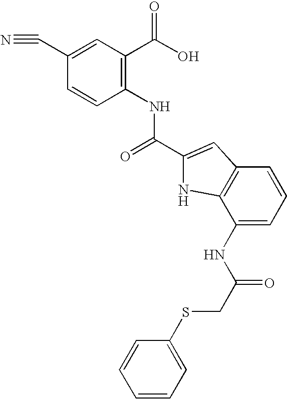 |
| 1.97 | 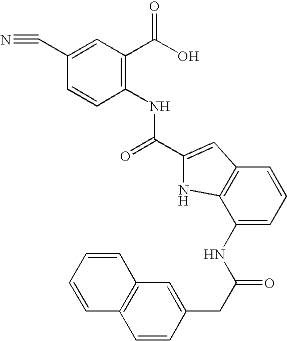 |
| 1.98 | 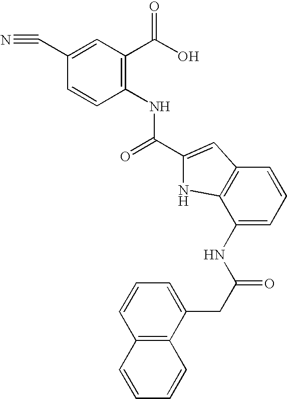 |
| 1.99 | 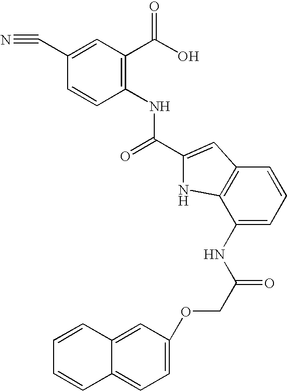 |
| 1.100 | 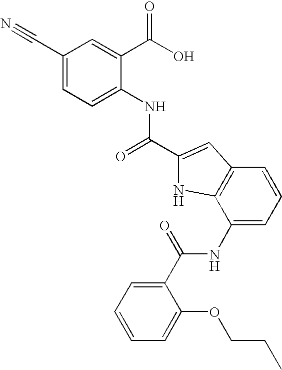 |
| 1.101 | 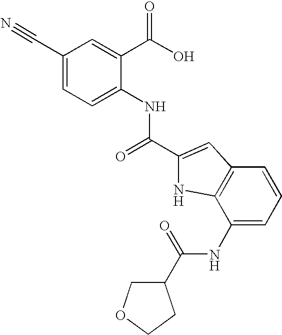 |
| 1.102 | 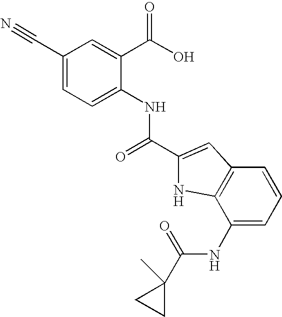 |
| 1.103 | 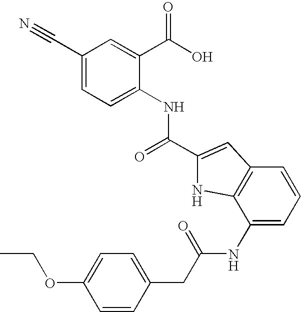 |
| 1.104 | 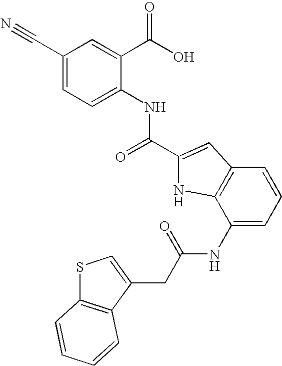 |
| 1.105 | 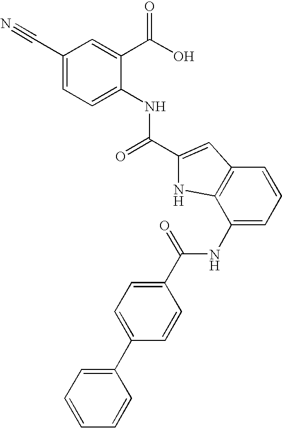 |
| 1.106 | 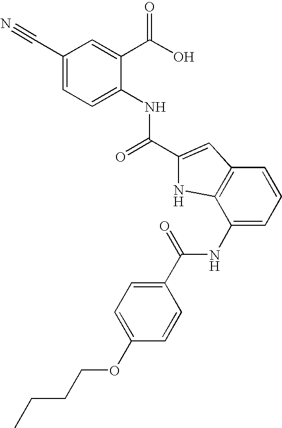 |
| 1.107 | 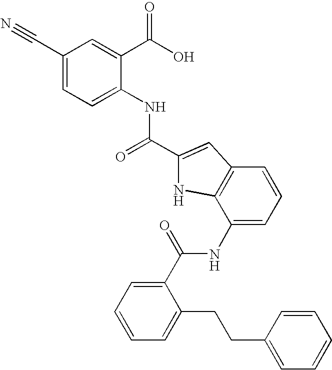 |
| 1.108 | 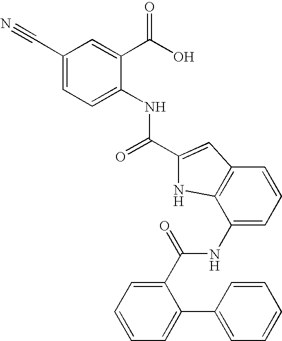 |
| 1.109 | 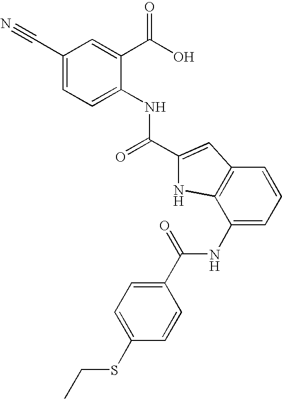 |
| 1.110 | 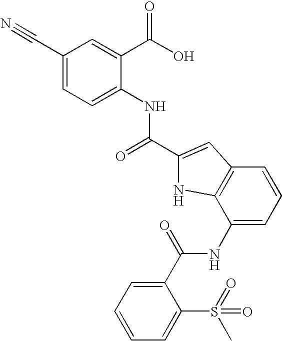 |
| 1.111 | 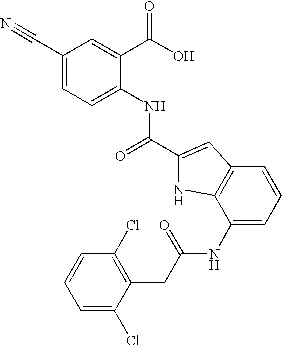 |
| 1.112 | 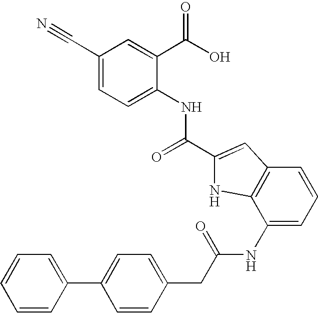 |
| 1.113 | 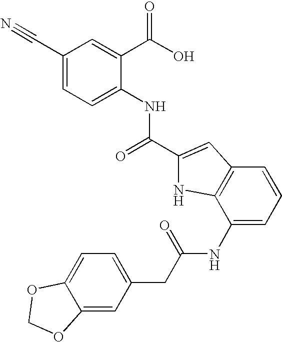 |
| 1.114 | 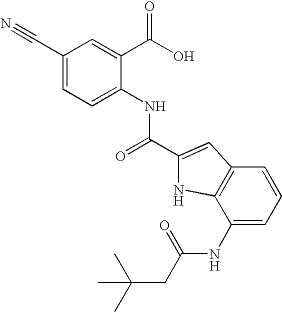 |
| 1.115 | 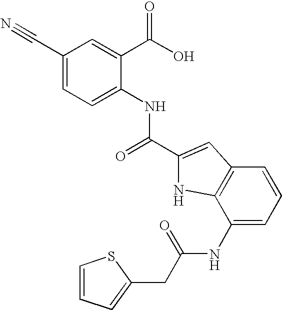 |
| 1.116 | 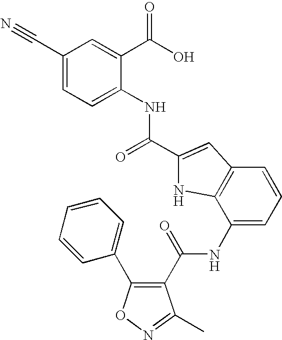 |
| 1.117 | 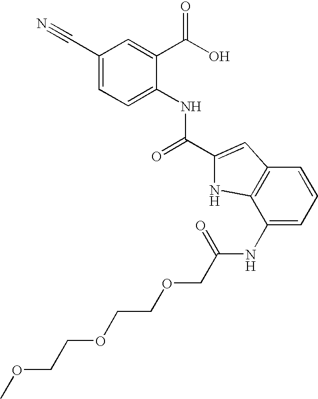 |
| 1.118 | 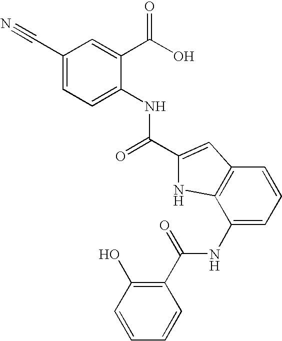 |
| 1.119 | 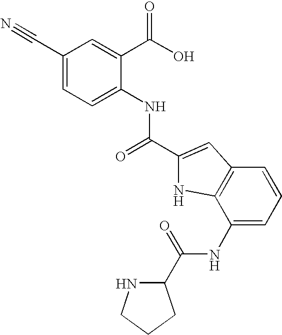 |
| 1.120 | 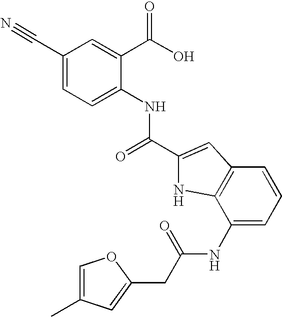 |
| 1.121 | 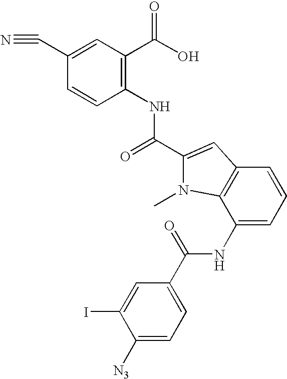 |

### Example 2: R₄ as Benzothiazine and Derivatives thereof

Preparation of 2-(4-Chlorophenyl)-3,4-dihydro-2H-1,2-benothiazine-7-carboxylic acid 1,1-dioxide

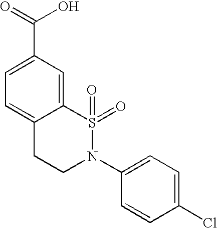

Chlorosulfonic acid (25 mL, 380 mmol, Aldrich) was added to a flask containing 4-(2chloroethyl)benzoic acid (5.00 g, 27.1 mmol, Lancaster), and the mixture was heated in an 80° C. oil bath for 4.3 hours. The mixture was then poured over ice. The precipitate was extracted into 2×200 mL of CH₂Cl₂ to which a small amount of THF was added to help solubility. The organics were dried over MgSO₄ and evaporated leaving 6.82 g of brown solid. To 3.95 g of this sulfonyl chloride was added 4-chloro-N-methylaniline (3.65 g, 28.6 mmol) and toluene (60 mL). Triethylamine (2.0 mL, 14 nmuol, Aldrich) was added, and the mixture was heated in a 100° C. oil bath for 4.25 hours. A solution of 3 M aqueous sodium hydroxide (25 mL) was added, and the mixture was heated for a further 15 minutes. The mixture was then added to a separatory funnel with 50 mL of water and 50 mL of MTBE. The aqueous layer was washed with an additional 100 mL of MTBE and then made acidic with concentrated HCl. Product was extracted into 2×150 mL of CH₂Cl₂. The combined CH₂Cl₂ was dried over MgSO₄ and evaporated leaving 1.75 g of brown solid that was carried on without further purification. For the purposes of characterization, 340 mg of the product was recrystallized from hot toluene/THF. The crystals were washed with toluene and dried at 105 OC under vacuum yielding 76 mg of light brown solid.

### Example 2.1 Methyl 2-({[2-(4-chlorophenyl)-1,1-dioxido-3,4-dihydro-2H-1,2benzothiazin-7-yl]carbonyl}amino)-5-cyanobenzoate

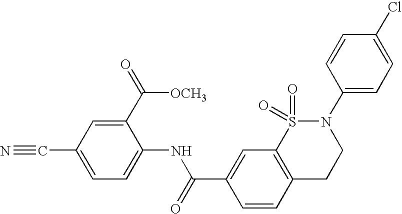

To 2-(4-chlorophenyl)-3,4-dihydro-2H-1,2-benzothiazine-7-carboxylic acid 1,1-dioxide (705 mg, 2.09 mmol) in CH₂Cl₂ (30 mL) was added DMF (20 μL) and oxalyl chloride (300 mL, 3.4 mmol). The mixture was stirred for 3.5 hours, and the solvent and excess oxalyl chloride were removed by rotary evaporation. The residue was dissolved in CH₂Cl₂ (15 mL), and methyl 2-amino-5-cyanobenzoate (310 mg, 1.76 rnmol) in pyridine (6 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with CH₂Cl₂ as eluent. Yield was 372 mg of white solid.

### Example 2.2 2-({[2-(4-Chlorophenyl)-1,1-dioxido-3,4-dihydro-2H-1,2benzothiazin-7-yl]carbonyl}amino)-5-cyanobenzoic acid

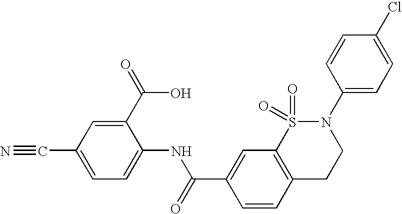

To a mixture of the corresponding methyl ester (276 mg, 0.556 mmol) in dioxane (20 mL) was added 1 M aqueous sodium hydroxide (1.0 mL). The mixture was stirred at room temperature for 1.5 hours and then at 50° C. for 20 minutes. The reaction mixture was added to a separatory fumnel with 100 mL of 1 M aqueous HCl, and the product was extracted into 100 mL of EtOAc with a small amount of THF added. The organics were washed with an additional 100 mL of 1 M aqueous HCl followed by 100 mL of water. They were then dried over MgSO₄ and evaporated. The residue was recrystallized from hot ethanoIITHF. The solids were washed with ethanol and then dried at 100° C. under vacuum yielding 116 mg of white solid. ¹H NMR (400 MHz, DMSO-d₆) δ 12.53 (s, 1H), 8.78 (d, J=8.8 Hz, 1H), 8.40 (d, J=2.1 Hz, 1H), 8.32 (d, J=1.8 Hz, 1H), 8.18 (dd, J=8.2,1.9 Hz, 1H), 8.11 (dd, J=8.8,2.1 Hz, 1H), 7.75 (d,J=8.2 Hz, 1H), 7.46 (d,J=8.8 Hz, 2 H), 7.28 (d,J=8.8 Hz, 2 H), 4.23 (t, J=6.3 Hz, 2 H), 3.29 (t, J=6.3 Hz, 2 H).

### Example 3: R₄ as Pyridine or Derivatives thereof

Pyridine Synthesis

The synthesis of the pyridine derivative is outlined in Scheme 1. The amide 1 was easily prepared by acylation of 4-chloronicotinic acid. The displacement of the chloride with a thiol nucleophile could be achieved with thioacetate in refluxing methanol affording the desired thiol in modest yield. The conversion of the thiol to a sulfonamide and subsequent hydrolysis afforded the desired acid.

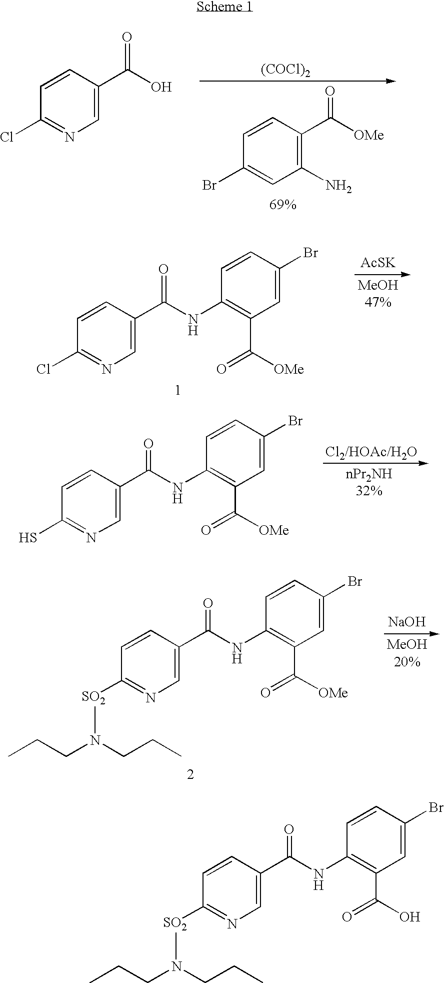

Preparation of 5-Phenoxynicotinonitrile

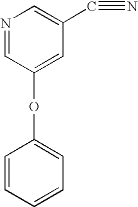

5-Bromonicotinonitrilel (915 mg, 5 nmiol) and sodium phenoxide (697 mg, 6 nmuol, Aldrich) were suspended in dry DMSO (10 mL) and heated to 120° C. for 10 hours. The reaction had stalled at this point and was diluted with EtOAc (200 mL). The organic mixture was washed 2× with 1.0 M HCl, 1× with 1.0 M NaOH, 1× with water and 1× with brine (175 mL each). The organic layer was dried over MgSO₄, filtered and evaporated. The resultant dark oil was purified on a Biotage Flash 40M (90 g) silica cartridge using 100% CH₂Cl₂. After evaporation the resultant mnilky oil became a solid and was dried under vacuum at 100° C. to afford 225 mg (23%) of an off-white solid. ¹H NMR (400 MHz, CDCl₃) δ 8.60 (dd, J=6.3, 2.2 Hz, 2 H), 7.42-7.48 (m, 3 H), 7.25-7.30 (m, 1H), 7.05-7.09 (m, 2 H).

Preparation of 5-Phenoxynicotinic acid

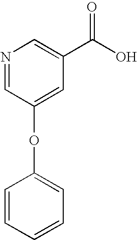

5-Phenoxynicotinonitrile (196 mg, 1.0 mmol) was dissolved in EtOH (2 mL), and the solution was treated with NaOH (800 mg, 20.0 nmmol) in water (10 miL). The colorless solution was refluxed for one hour then stirred at RT overnight. After evaporating the EtOH, the aqueous solution was diluted to 100 mL with water and the solution washed 2× with CH₂Cl₂. The aqueous layer was made acidic with concentrated HCl to afford a white precipitate, which was collected by vacuum filtration, washed with water and heptane and dissolved in a mixture of CH₂Cl₂ and THF. The organic solution was dried over MgSO₄, filtered and evaporated. The resultant product was dried at 100° C. under vacuum to afford 79g (37%) of a white solid. ¹H NMR (400 MHz, CD₃OD+CHCl₃) δ 8.93 (s, 1H), 8.52 (s, 1H), 7.96 (d, J=1.5 Hz, 1H), 7.40-7.46 (m, 2 H), 7.22-7.27 (m, 1H), 7.07-7.10 (m, 2 H), 4.30 (br s, 1H).

### Example 3.1: Methyl

5-cyano-2-{[(5-phenoxypyridin-3-yl)carbonyl]amino}benzoate

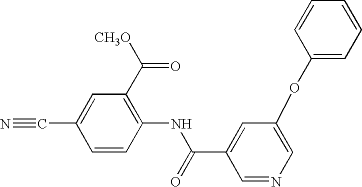

5-Phenoxynicotinic acid (75 mg, 0.35 mnmol) was suspended in dry CH₂Cl₂ (10 mL) under N₂ and treated with DMF (15 μL) followed by oxalyl chloride (60 μL, 0.70 nmol). Gas evolved as the mixture rapidly became homogenous. After stirring for one hour at RT, the solvent and excess oxalyl chloride were evaporated and the resultant yellowish residue was taken up in dry CH₂Cl₂ (10 mL). Methyl 2-amino-5-cyanobenzoate (61 mg, 0.35 mmol) was added as a solution in dry pyridine (3 mL) and the dark amber solution was stirred at RT overnight. The reaction was poured into CH₂Cl₂ (100 mL) and washed 2× with 1.0M HCl and 1× with brine (75 muL each). The solvents were evaporated and the crude product was purified on a Biotage Flash 40M (90 g) silica cartridge using a step gradient of CH₂Cl₂ to 5% EtOAc in CH₂Cl₂. After evaporation the resultant solid was dried under vacuum at 100° C. to afford 68 mg (52%) of a white solid. ¹H NMR (400 MHz, CDCl₃) δ 12.37 (s, 1H), 9.02 (d, J=8.9 Hz, 1H), 8.99 (br s, 1H), 8.61 (br, s, 1H), 8.41 (d, J=1.9 Hz, 1H), 7.83-7.88 (m, 2 H), 7.45 (t,J=7.9 Hz, 2 H), 7.25 (t,J=7.4Hz, 1H), 7.12 (d,J=7.7 Hz, 2 H), 4.01 (s, 3 H).

### Example 3.2: 5-Cyano-2-{[(5-phenoxypyridin-3-yl)carbonyl]amino}benzoic acid

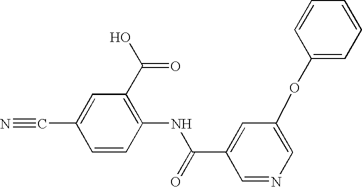

The corresponding methyl ester (example 3.1, 54 mg, 0.14 rmmol) was dissolved in dioxane (10 mL), and the solution was treated with 1.0 M LiOH (1.0 mL, 1.0 nmmol) turning the solution a light yellow color. After stirring overnight at RT, the reaction was complete by HPLC and the solution was poured into EtOAc (100 mL). The organic layer was washed 2× with 1.0 M HCl, 4× with water and 1× with brine (75 riL each). The organic layer was dried over Na₂SO₄ and following evaporation of solvent the resultant white solid was re-crystallized from hot CH₃OH/TBF. A second crop of crystals was similarly obtained from the mother liquor. The resultant products were combined and dried at 100° C. under vacuum to afford 17 mg (33%) of straw colored crystals. (2.5% HCl salt). ¹H NMR (400 MHz, DMSO-d₆) δ 12.41 (s, 1H), 8.90 (d, J=1.2 Hz, 1H), 8.72 (d, J=8.7 Hz, 1H), 8.65 (d, J=2.7 Hz, 1H), 8.40 (d, J=1.9 Hz, 1H), 8.11 (dd, J=8.7, 1.9 Hz, 1H), 7.79 (t, J=2.1 Hz, 1H), 7.48 (t, J=7.9 Hz, 2 H), 7.27 (t, J=7.4 Hz, 1H), 7.18 (d, J=7.7 Hz, 2 H).

Preparation of 5-(Ethylthio)nicotinonitrile

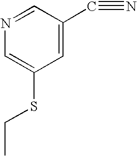

5-Bromonicotinonitrilel (1.83 g, 10 mmol), sodium ethanethiolate (799 mg, 9.5 mmol, Aldrich) and a dash of sodium ethoxide were suspended in dry DMF (20 mL) and stirred at RT. A moderate exotherm was noted after five minutes and the reaction was judged complete after 2 hours. The reaction was poured into MTBE (200 mL), and was washed 6× with water, 1× with 1.0 M NaOH, 1× with 1.0 M HCl, and 1× with brine (175 mL each). The organic layer was evaporated and the resultant oil was purified on a Biotage Flash 40M (90 g) silica cartridge using 0.75% EtOAc in CH₂Cl₂. After evaporation the resultant colorless oil became a solid and was dried under vacuum at RT to afford 848 mg (54%) of an off-white solid. ¹H NMR (400 MHz, DMSO-d₆) δ 8.79 (d, J=1.9 Hz, 1H), 8.77 (d, J=2.3 Hz, 1H), 8.28-8.40 (m, 1H), 3.13 (q, J=7.3 Hz, 2 H), 1.26 (t, J=7.4 Hz, 3 H).

Preparation of 5-(Ethylthio)nicotinic acid

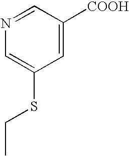

5-(Ethylthio)nicotinonitrile (569 mg, 3.47 nmuol) was dissolved in EtOH (10 mL), and the solution was treated with NaOH (2.77 g, 69.3 mmol) in water (20 mrL). The colorless solution was refluxed for one hour during which time the solution became straw colored. After evaporating the EtOH, the aqueous solution was diluted to 175 mL with water and the solution washed 2× with CH₂Cl₂ (200 μL each). The aqueous layer was made acidic with concentrated HCl and extracted 3× with EtOAc. The combined organics were dried over MgSO₄, filtered and evaporated. The resultant white solid was dried at 100° C. under vacuum to afford 561 g (88%) of white solid. ¹H NMR (400 MHz, DMSO-d₆) δ 13.57 (s, 1H), 8.85 (d, J=1.9 Hz, 1H), 8.72 (d, J=2.3 Hz, 1H), 8.11 (t, J=2.2 Hz, 1H), 3.10 (q, J=7.3 Hz, 2 H), 1.25 (t, J=7.3 Hz, 3 H).

### Example 3.3: 5-Cyano-2-({[5-(ethylthio)pyridin-3-yl]carbonyl}amino)benzoic acid

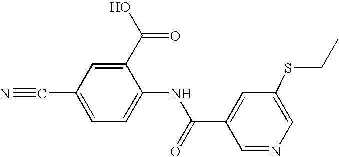

5-(Ethylthio)nicotinic acid, (1.95 g, 10.6 mmol) was suspended in dry CH₂Cl₂ (30 mL) under N₂ and treated with DMF (25 ,uL) followed by oxalyl chloride (1.86 mL, 21.3 mmol). Gas evolved as the mixture rapidly became homogenous. After stirring for one hour at RT, the solvent and excess oxalyl chloride were evaporated and the resultant residue was taken up in CH₂Cl₂ (30 mL). tert-Butyl-2-amino-5-cyanobenzoate (2.23 g, 10.7 mmol) was added as a solution in 5 mL dry pyridine and the dark gold solution was stirred at RT for four hours. The cloudy reaction was diluted with CH₂Cl₂ (200 mL), and the organic layer was washed 2× with 1.0 M HCl, and 1× with brine (200 mL each). The organic layer was evaporated, and then purified on a Biotage Flash 40M (90g) silica cartridge using 5% EtOAc in CH₂Cl₂. The solvent was evaporated and the resultant product dried under vacuum at 100° C. to afford 2.89 g (71%) of white solid as the t-butyl ester. The t-butyl ester (1.0 g, 2.61 mmol) was dissolved dry CH₂Cl₂ (10 mL) and was treated with TFA (5.00 mL) turning the mixture yellow. After stirring 24 hours at RT, the reaction was complete by HPLC. The crude product was precipitated by diluting the reaction with CH₃OH and collected by vacuum filtration thru #42 filter paper. The product was washed with CH₃OH, THF, and heptane. The crude product was dried on the filter paper at 100° C. under vacuum to afford 626 mg (73%) of bone white solid. ¹H NMR (400 MHz, DMSO-d₆) δ 12.39 (s, 1H), 8.89 (d, J=2.1 Hz, 1H), 8.76 (d, J=8.5 Hz, 1H), 8.76 (d, J=2.3 Hz, 1 H), 8.41 (d, J=2.1 Hz, 1H), 8.18 (t, J=2.1 Hz, 1H), 8.12 (dd, J=8.7, 2.1 Hz, 1 H), 3.13 (q,J=7.4 Hz, 2 H), 1.29 (t,J=7.3 Hz, 3 H).

### Example 3.4: 5-Cyano-2-{[(6morpholin-4-ylpyridin-3-yl)carbonyl]amino}benzoic acid

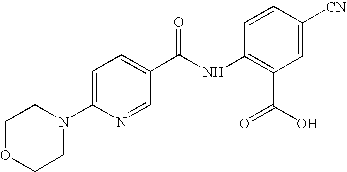

t-butyl 2-{[(6-chloropyridin-3-yl)carbonyl]amino}-5-cyanobenzoate (100 mg, 0.28 mmol), morpholine (73 mg, 0.84 mmol) and AgNO₃ (51 mg, 0.28 mmol) were refluxed in Ethanol (30 ml) for 24 h. The resulting misture was filtrated through celite. The solvent was removed and the residue was loaded on silica gel to be purified by flash chromatography (DCM/MeOH=50: 1) to afford 110 mg (95%) of advanced t-butyl ester, 105 mg of which was hydrolyzed by TFA (2 ml) in DCM (8 ml) to afford 81 mg (89%) of a yellow solid.

Analytical data ¹H NMR (300 MHz, DMSO-d₆) δ 12.28 (s, 1H), 8.84 (d, J=8.9 Hz, 1H), 8.72 (d, J=2.3 Hz, 1H), 8.39 (d, J=2.1 Hz, 1H), 8.07 (dd, J=2.1, 8.9 Hz, 1H), 8.02 (dd, J=2.6, 9.1 Hz, 1H), 7.0 (d, J=8.9 Hz, 1H), 3.67 (m, 8H);

### Example 3.5: 5-Cyano-2-{[(6-piperidin-1-ylpyridin-3-yl)carbonyl]amino}benzoic acid

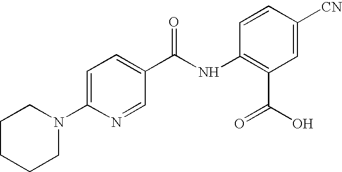

t-butyl 2-{[(6-chloropyridin-3-yl)carbonyl]amino}-5-cyanobenzoate (100 mg, 0.28 minol) and piperidine (73 mg, 0.84 minol) afforded 86 mg (76%) of advanced t-butyl ester, 105 mg of which was hydrolyzed by TFA (2 ml) in DCM (8 ml) to afford 62 mg (90%) of a yellow solid.

Analytical data

¹H NMR (300 MHz, DMSO-d₆) δ 12.23 (s, 1H), 8.84 (d, J=9.0 Hz, 1H), 8.67 (d, J=2.4 Hz, 1H), 8.38 (d, J=2.0 Hz, 1H), 8.05 (dd, J=2.1, 8.9 Hz, 1H), 7.95 (dd, J=2.5, 9.1 Hz, 1H), 6.95 (d, J=9.2 Hz, 1H), 3.68 (t, J=5.4 Hz, 4 H), 1.65 (m, 2 H), 1.55 (m, 4 H).

### Example 3.6: Methyl 2-({14-(benzyloxy)pyridin-2-yl]carbonyl}amino)-5-bromobenzoate

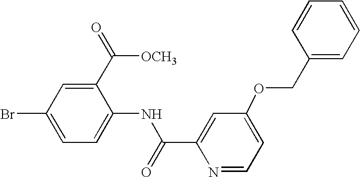

To sodium 4-(benzyloxy)pyridine-2-carboxylate, as described by Clark-Lewis et al. in J Chem. Soc. 1961, 189-201, (2.02 g, 8.04 rnmol) in CH₂Cl₂ (100 mL) was added DMF (50 μL) and oxalyl chloride (1.4 mL, 16 minol). The mixture was stirred for 1 hour, and the solvent and excess oxalyl chloride were removed by rotary evaporation. The residue was suspended in CH₂Cl₂ (75 mL), and methyl 2-amino-5-bromobenzoate (1.50 g, 6.52 mmol, Avocado) in pyridine (20 mL) was added. The mixture was stirred overnight and then concentrated by rotary evaporation. The residue was added to a separatory funnel with 150 mL of CH₂Cl₂, and this solution was washed with 100 mL of saturated NaHCO₃, 100 mL of 4% aqueous acetic acid, 100 mL of saturated NaHCO₃, and 100 mL of brine. The CH₂Cl₂ was evaporated in the presence of silica gel, and the product was purified by chromatography using a Biotage Flash 40 M silica cartridge with a gradient from 50 % CH₂Cl₂ in heptane to 75% CH₂Cl₂ in heptane as eluent. Yield was 1.52 g of white solid.

### Example 3.7: 2-({14-(Benzyloxy)pyridin-2-yl]carbonyl}amino)-5-bromobenzoic acid

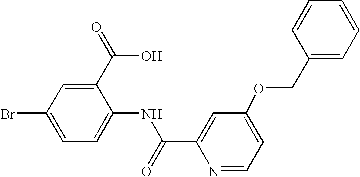

To a mixture of the methyl ester from example 3.6 (356 mg, 0.807 mmol) in dioxane (20 mL) was added 1 M aqueous sodium hydroxide (2.0 mL). The mixture was stirred at room temperature for 4 hours and then in a 50° C. oil bath for 1 hour. The reaction mixture was added to a separatory fumnel with 100 mL of 1 M aqueous HCl, and the product was extracted into 100 mL of EtOAc. The EtOAc was washed with an additional 100 mL of 1 M aqueous HC1 followed by 100 mL of water. It was then dried over MgSO₄ and evaporated. The residue was recrystallized from hot ethanol/THF. The solids were washed with ethanol followed by heptane and then dried at 100° C. under vacuum yielding 249 mg of white solid. ¹H NM (400 MHz, DMSO-d₆) δ 13.00 (s, 1H), 8.81 (d, J=9.1 Hz, 1H), 8.56 (d, J=5.8 Hz, 1H), 8.13 (d, J=2.5 Hz, 1H), 7.87 (dd, J=9.1, 2.5 Hz, 1H), 7.76 (d, J=2.5 Hz, 1H), 7.50 (d, J=6.8 Hz, 2 H), 7.43 (t, J=7.2 Hz, 2 H), 7.37 (t, J=7.2 Hz, 1H), 7.32 (dd, J=5.7, 2.6 Hz, 1H), 5.33 (s, 2 H).

### Example 3.8: 2-({[4-(Benzyloxy)-1-oxidopyridin-2-yl]carbonyl}amino)-5-bromobenzoic acid

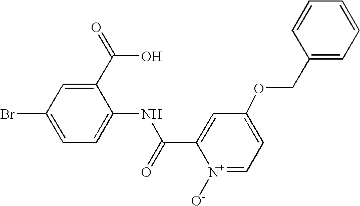

To a solution of methyl 2-({[4-(benzyloxy)pyridin-2-yl]carbonyl}amino)-5-bromobenzoate (262 mg, 0.593 mmrol) in 1,2-dichloroethane (10 mL) was added solid m-CPBA (225 mg, 1.30 mmol, Aldrich). The mixture was stirred at room temperature for 3 hours, at 55° C. for 6.5 hours, and then at 75° C. for 8.5 hours. An additional portion of m-CPBA (302 mg, 1.75 mmol) was added, and the mixture was heated at 75° C. for an additional 15 hours. The solution was diluted with 100 mL of CH₂Cl₂ and then washed with 100 mL of saturated NaHCO₃. The CH₂Cl₂ was dried over MgSO₄ and evaporated. The residue was dissolved in CH₂Cl₂ and loaded onto a 1″ plug of silica gel. Unreacted starting material was eluted with CH₂Cl₂, and the desired N-oxide was eluted with EtOAc. Yield was 137 mg of white solid as the methyl ester. To a solution of the corresponding methyl ester (137 mg, 0.300 mmol) in THF (30 mL) in a 50° C. oil bath was added sodium hydroxide (0. 50 mL of 1.0 M solution in water). A precipitate formed within minutes. An additional portion of sodium hydroxide solution (1.0 mL) was added after 1 hour. Heat was removed after an additional 4.2 hours. The reaction mixture was stirred ovenight with 100 mL of 1.0 M aqueous HCl and 100 mL of CH₂Cl₂, but the solids did not dissolve. The solids were then filtered, washed with water followed by heptane, and dried at 100° C. under vacuum. Yield was 63 mg of white solid. ¹H NMR (400 MHz, DMSO-D6) δ ppm 5.31 (s, 2 H) 7.40 (m, 4 H) 7.49 (m, 2 H) 7.81 (dd, J=8.98, 2.59 Hz, 1H) 7.89 (d, J=3.65 Hz, 1H) 8.01 (d, J=2.74 Hz, 1H) 8.39 (d, J=7.00 Hz, 1H) 8.54 (d, J=8.83 Hz, 1H) 13.68 (s, 1H) 14.73 (s, 1H).

### Example 3.9: 2-({[4-(Benzyloxy)pyridin-2-yl]carbonyl}amino)-5-cyanobenzoic acid

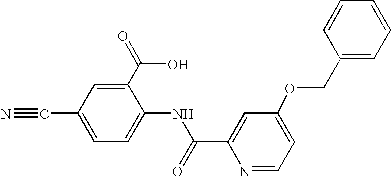

To a slurry of sodium 4-(benzyloxy)pyridine-2-carboxylate (2.58 g, 10.3 mmol) in CH₂Cl₂ (100 mL) was added DMF (50 ,uL) followed by oxalyl chloride (1. 8 mL, 21 mmol). It became a solution. Solvent and excess oxalyl chloride were removed by rotary evaporation after 1 hour. The residue was dissolved in CH₂Cl₂ (75 mL), and methyl 2-amino-5-cyanobenzoate (1.61 g, 9.14 nmmol) in pyridine (15 mL) was added. The mixture was stirred for 2 days and then added to a separatory finnel with 100 mL of CH₂Cl₂. This solution was washed with 2×100 of 1.0 M citric acid and 100 mL of brine. Product was adsorbed onto silica gel and purified on a Biotage Flash 40 M silica gel cartridge using a gradient from 50% CH₂Cl₂ in heptane to 100% CH₂Cl₂. Product was collected as 296 mg of white solid as the methyl ester. To a mixture of the corresponding methyl ester (134 mg, 0.346 mmol) in dioxane (10 mL) was added 1 M aqueous sodium hydroxide (1.0 mL). The mixture was stirred at room temperature for 7 hours and then added to a separatory fumnel with 100 mL of 1 M aqueous HCl. The product was extracted into 100 mL of EtOAc. The EtOAc was washed with 100 mL of water. It was then dried over MgSO₄ and evaporated. The residue was recrystallized from hot ethanol/THF. The solids were washed with ethanol followed by heptane and then dried at 100° C. under vacuum yielding 68 mg of white solid. ¹H NMR (400 MHz, DMSO-D6) δ ppm 5.34 (s, 2 H) 7.34 (dd, J=5.85, 2.63 Hz, 1H) 7.38 (d, J=7.31 Hz, 1H) 7.43 (t, J=7.16 Hz, 2 H) 7.50 (d, J=7.02 Hz, 2 H) 7.78 (d, J=2.63 Hz, 1H) 8.11 (dd, J=8.77, 2.05 Hz, 1H) 8.41 (d, J=2.05 Hz, 1H) 8.57 (d, J=5.55 Hz, 1H) 9.00 (d,J=8.77 Hz, 1H) 13.29(s, 1H) 14.16 (s, 1H)

### Example 4: R₄ as Nalidixic Acid or Derivative Thereof

Preparation of of Nalidixic Acid Chloride (27)

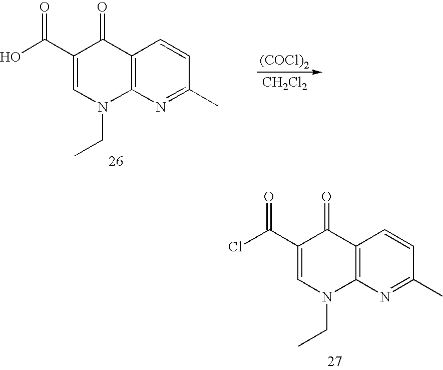

To a solution nalidixic acid (26, 5.8 g, 0.025 mol dissolved 250 mL of CH₂Cl₂) was added DMF (2 drops) and oxalyl chloride (25 mL of a 2 M solution in CH₂Cl₂, 50 mmol) under a nitrogen atmosphere. After stirring for 20 h, the acid chloride was concentrated to dryness, azeotroped with toluene (2×25 mL), dried on a high vacuum overnight, and used in situ for the following reactions.

### Example 4.1: Solid Phase Synthesis of 5-Cyano-2-[(1-ethyl-7-methyl-4-oxo-1,4dihydro-11,8]naphthyridine-3-carbonyl)-amino]benzoic Acid (28)

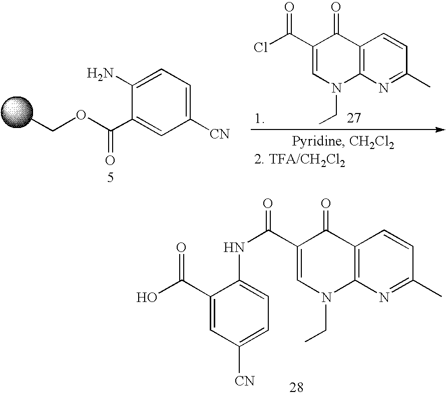

Resin-bound

5-cyano anthranilic acid (5, 0.5 g, 1.0 inmolvg loading, 500 μmol) was suspended in pyridine (100 mL) in a 250-mL serum flask equipped with an overhead stirrer. After the addition of nalidixic acid chloride (27, 5 μL of a 1 M solution in CH₂Cl₂, 10 equiv), the flask was purged with nitrogen and stirred at room temperature for 20 h. The reaction mixture was then drained and the resin washed (CH₃CN, DMF, CH₃CN, DMF, CH₃CN, DMF, H₂O, THF, H₂O, THF, H₂O, THF, CH₃CN, CH₂Cl₂, CH₃CN, CH₂Cl₂, CH₃CN, CH₂Cl₂, CH₂Cl₂, CH₂Cl₂, 50 mL each wash) was then cleaved from the resin using 50% TFA/CH₂Cl₂ for 3 h to yield 28 (60 mg, 32%) in 78% reaction purity as determined by HPLC/MS analysis.

### Example 4.2: Solution Phase Synthesis of 5-Cyano-2-[(1-ethyl-7-methyl-4-oxo-1,4-dihydro-[1,8

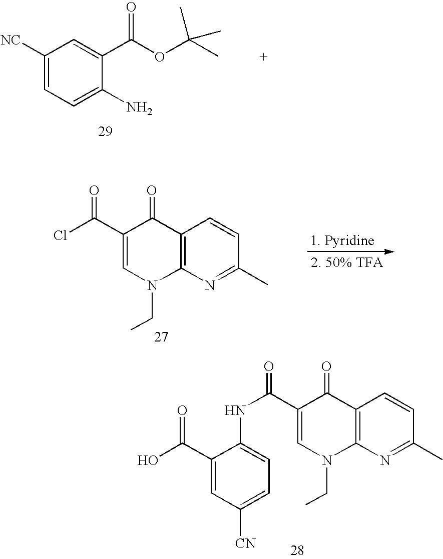

To a 250-mL round bottom flask containing t-butyl-2-amino-5-cyano-benzoate⁸ (29, 100 mg, 459 Hmol, PHA-561053, lot 34629-tjb-145) was added nalidixic acid chloride (27, 6 mL of a 1 M solution in CH₂Cl₂, 1.2 equiv) and pyridine (75 mL). Additional nalidixic acid chloride (O.5 equiv) was added until the aniline was completely consumed as deterrnined by HPLC analysis of the reaction mixture. The reaction mixture was concentrated to a dark brown solid and dried on a high vacuum for 4 h. The solid was dissolved in CH₂Cl₂ (250 mL) and extracted with 2 N NaOH (3×250 mL). The combined organic layers were dried over MgSO₄ and concentrated to yield a brown solid. The crude product was dissolved in CH₂Ck₂ (100 mL) and stirred with a 50% TFA/CH₂Cl₂ solution (50 mL) for 24 h. The reaction mixture was then concentrated and the crude solid triturated with CH₂Cl₂ (5 mL) to yield 67.3 mg (39%) of the corresponding amide (28) in 96% purity: ¹H NMR (DMSO-d₆) δ 13.20 (s, 1H), 9.11 (s, 1H), 8.82 (d, J=8.84 1H), 8.27 (d, J=2.08, 1H), 8.01 (dd, J=2.08, 8.82, 1H), 7.51 (d, J=8.17, 1H), 4.61 (q, J=6.87), 2.68 (s, 3H), 1.43 (t, J=7.03).

### Example 5: R₄ as Pyrazole or Derivatives Thereof

Pyrazole Synthesis

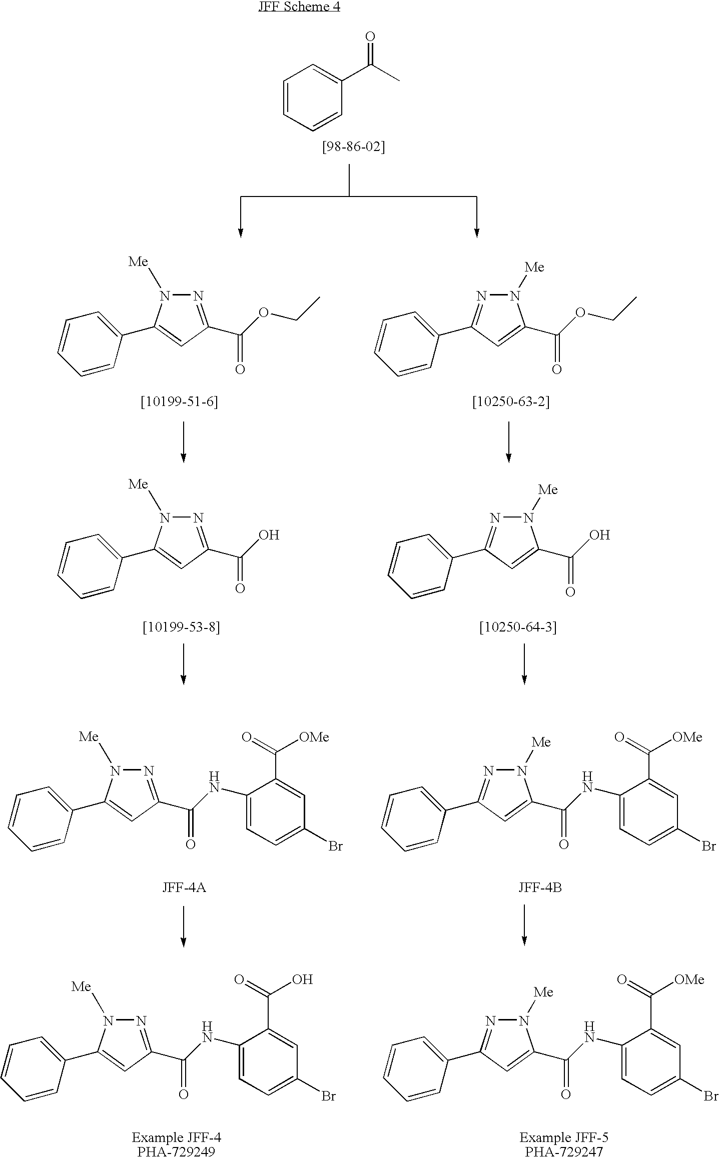

Preparation of 1-Methyl-5-phenyl-1H-pyrazole-3-carboxylic acid ethyl ester [10199-51-61 and 1-MethyW3-phenyl-1H-pyrazole-5-carboxylic acid ethyl ester [10250-63-2].

To a mixture of [98-86-2] (50 mL, 0.42 mol) and NaH (60% oil dispersion, 17.5 g, 0.44 mol) in EtOH (1 L) is added diethyloxalate (36.5 mL, 0.42 mol). The reaction mixture is stirred overnight at rt. It is concentrated to give 85 g (92%) of a yellow powder. To a solution of this powder (3.15 g, 14.3 rmmol) in a mixture of EtOH (50 mL) and aqueous 3 M HCl (5 mL) is added methylhydrazine (0.76 mL, 14.3 mmol). The reaction mixture is stirred overnight. It is concentrated, and the residue is purified by silica chromatography to give the two pyrazole products. [10199-51-6]: R. =0.8 (4:1 hexanes/EtOAc); ¹H NMR (CDC1₃, 400 MHz) δ7.84 (2H), 7.45 (2H), 7.35 (1H), 4.42 (2H), 4.28 (3H), 1.45 (3H); MS (ESI+) m/z 231.2. [10250-63-21: Rf=0.3 (4:1 hexanes/EtOAc); ¹H NMR (CDCl₃, 400 MHz) 6 7.4 (5H), 6.90 (1H), 4.62 (2H), 4.00 (3H), 1.44 (3H); MS (ESI+) m/z 231.4.

Preparation of 1-Methyl-5-phenyl-1H-pyrazole-3-carboxylic acid 110199-53-8].

Using 1.2 g of [10199-51-61, and the procedure described for the preparation of [10250-64-3], acid [10199-53-81 is isolated as a powder: MS (ESI−) m/z 185.2.

Preparation of 1-Methyl-3-phenyl-1H-pyrazole-5-carboxylic acid [10250-64-3].

A mixture of ester [10250-63-21 (1.7 g, 7.4 mmol) and aqueous 1 M KOH (10 mmol) in EtOH (20 mL) is stirred for 6 h. The reaction mixture is concentrated, and the residue is partitioned between cold aqueous 1 M HCl and EtOAc. The EtOAc solution is separated. The aqueous solution is further extracted with EtOAc. The combined EtOAc extracts are dried and concentrated to give [10250-64-31 as a powder: MS (ESI−) n/z 185.5.

Preparation of 5-Bromo-2-[[(1-methyl-3-phenyl-1H-pyrazol-5-yl)carbonyl]amino]benzoic acid methyl ester (JFF-4b).

To a suspension of acid [10250-64-3] (150 mg, 0.74 mmol) in CH₂Cl₂ is added oxalyl chloride (65 μL, 0.74 mmol) and DMF (1 drop). The reaction mixture is stirred at rt for 90 min. It is concentrated to give an oil. The oil is dissolved in CH₂Cl₂ (5 mL), and methyl 5-bromoanthranilate (17 mg, 0.74 mmol), NEt₃ (207 μL, 1.48 mmol), and DMAP (10 mg, 0.074 mol) are added. The reaction mixture is stirred at rt for 72 h. It is quenched with aqueous 1 M HCl (10 mL). The aqueous mixture is extracted with EtOAc. The EtOAc extracts are dried and concentrated to give an oil. The oil is dissolved in CH₂Cl₂, and 4b is precipitated as a solid by the addition of hexanes: MS (ESI+) m/z 415.3.

Preparation of 5-Bromo-2-[[(1-methyl-5-phenyl-1H-pyrazol-3-yl)carbonyl]amino]benzoic acid methyl ester (JFF-4a).

Compound 4a is prepared from [10199-53-8], using the procedure given for JFF-4b: MS (ESI+) m/z 415.5.

### Example 5.1: 5-Bromo-2- [I(1-methyl-5-phenyl-1H-pyrazol-3-yl)carbonyl]amino]benzoic acid (Example JFF-4).

### Example JFF-4 is prepared from 4a using the procedure given for Example JFF-5: MS (ESI+) m/z 399.2.

### Example 5.2: 5-Bromo-2-[[(1-methyl-3-phenyl-1H-pyrazol-5-yl) carbonyl]amino]benzoic acid (Example JFF-5).

To a solution of ester JFF-4b (75 mg, 0.18 mmol) in 2:1 MeOH/CH₂Cl₂ (3 mL) is added aqueous 1 M KOH (200 EL). The reaction mixture is stirred for 4 h. It is concentrated. The residue is partitioned between aqueous 1 M HCl and EtOAc. The EtOAc solution is separated, and the aqueous solution is extracted fiuther with EtOAc. The combined EtOAc extracts are dried and concentrated to give Example JFF-5 as a solid: MS (ESI−) m/z 399.2.

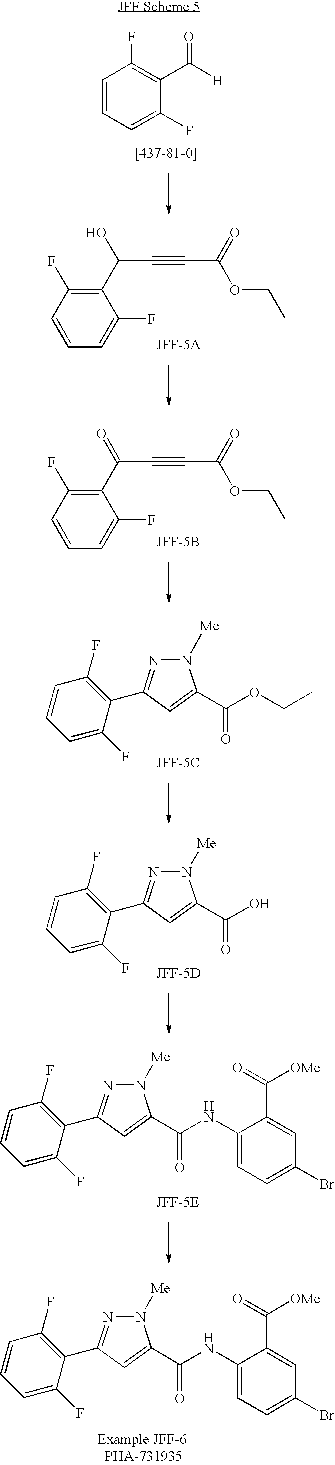

Preparation of (±)4-(2,6-Difluorophenyl)-4-hydroxybut-2-ynoic acid ethyl ester (JFF-5a).

A solution of LDA (2.0 M, 17.5 mL) is added to a solution of ethyl propiolate (3.6 mL, 0.035 mol) in THF (0.3 L) at -78° C. The reaction mixture is stirred for 30 min, at which time [437-81-0] (3.8 mL, 0.035 mol) is added. The reaction mixture is stirred at −78° C. for 30 rnin, and then is warmed to rt. It is poured into aqueous 5% CuSO₄ (0.2 L). The aqueous mixture is extracted with EtOAc (2×0.1 L). The combined EtOAc extracts are dried and concentrated to give an oil, that is purified by silica flash chromatography (9: 1 hexanes:EtOAc to give 5A: TLC Rf=0.2 (9: 1 hexanes/EtOAc); ¹H NMR (CDCl₃, 400 MHz) δ7.4 (1H), 7.0 (2H), 5.91 (1H), 4.3 (2H), 2.81 (1H), 1.35 (3H).

Preparation of 4-(2,6Difluorophenyl)-4-oxobut-2-ynoic acid ethyl ester (JFF5B).

To a solution of 5A (4.2 g, 17.5 mmol) in acetone (0.2 L) at 0° C. is added dropwise When the reaction is determined as complete by TLC (30 min), the reaction mixture is diluted with Et₂O and brine. The Et₂O extract is dried and concentrated to give an oil, that is purified by silica chromatography (4: 1 hexanes:EtOAc) to give 5B.

Preparation of 3-(2,6Difluorophenyl)-1-methyl-1H-pyrazole-5-carboxylic acid ethyl ester (JFF-5C, C₁₃H₁₂F₂N_{2O}₂).

A mixture of methylhydrazine (228 μL, 4.28 mmol) and 5B (680 mg, 2.86 mmol) in EtOH (30 mL) is refluxed for 4 h. The reaction mixture is evaporated, and the residue is purified by silica chromatography (2:1 hexanes:EtOAc) to give 5C: ¹H NMR (CDCl₃, 400 MHz) 6 7.3 (1H), 7.2 (1H), 7.0 (2H), 4.4 (2H), 4.3 (3H), MS (FAB) m/z 267.0949; Anal.C 58.64, H 4.56, N 10.06.

Preparation of 3-(2,6Difluorophenyl)-1-methyl-1H-pyrazole-5-carboxylic acid (JFF-5D).

A mixture of ester 5C (200 mg, 0.75 mmol) and aqueous 1 M KOH (2 mL) in EtOH (3 mL) is stirred for 2 h at rt. The reaction mixture is concentrated, and the residue is partitioned between aqueous 1 M HCl and EtOAc. The EtOAc solution is separated, and the aqueous solution is further extracted withEtOAc. The combined EtOAc extracts are dried and concentrated to give 5D: ¹H NMR (CD₃OD, 400 MHz) 6 7.43 (1H), 7.10 (3H), 4.24 (3H).

5-Bromo-2-[l[3-(2,difluorophenyl)-1-methyl-1H-pyrazol-5-yl]carbonyl]amino]benzoic acid (Example JFF-6, C₁₈Hl₂BrF₂N₃O₃).

To a suspension of acid 5D (100 mg, 0.42 nmmol) in CH₂Cl₂ (5 mL) is added oxalyl chloride (73 μL, 0.84 numol). After 2 h the reaction mixture is concentrated, and the residue is dissolved in CH₂Cl₂ (5 mL). Methyl 5-bromoanthranilate (145 mg, 0.63 mnmol), Et₃N (176 μL, 1.26 mnmol), and DMAP (10 mg, 0.084 mmol) are added. The reaction mixture is stirred at rt for 24 h. It is poured into aqueous 1 M HCl (10 mL). The aqueous mixture is extracted with EtOAc. The EtOAc solution is dried and concentrated. The residue is purified by silica chromatography (4:1 hexanes/EtOAc) to give 5E, the methyl ester: ¹H NMR (CDC1₃, 400 MHz) δ 11.97 (1H), 8.78 (1H), 8.26 (1H), 7.73 (1H), 7.35 (1H), 7.23 (1H), 7.06 (2H), 4.38 (3H), 4.02 (3H); MS (ESI+) m/z 451.3. A mixture of 5E (50 mg) and aqueous 1 M KOH (2 mL) in 2:1 MeOH/CH₂Cl₂ (3 mL) is stirred at rt ror 2 h. The reaction mixture is concentrated, and the residue is partitioned between aqueous 1 M HC1 and EtOAc. The EtOAc solution is separated, and the aqueous solution is further extracted with EtOAc. The combined EtOAc extracts are dried and concentrated to give Example JFF-6: ¹H NMR (CDCl₃, 400 MHz) δ 12.29 (1H), 8.65 (1H), 8.2 (1H), 7.61 (1H), 7.25 (1H), 7.14 (1H), 6.95 (2H), 4.27 (3H), MS (FAB) ml/z 436.0105.

### Example 6: R₄ as Benzisoxazolyl of Derivatives Thereof

Benzisoxazolyl Synthesis

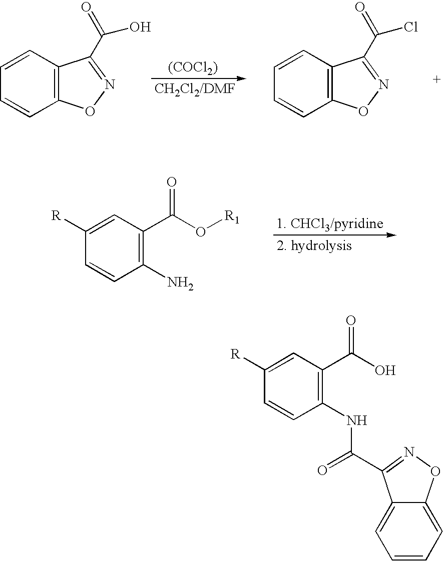

Preparation of 2-[(1,2-benzisoxazol-3-ylcarbonyl)aminol-5-cyanobenzoic acid

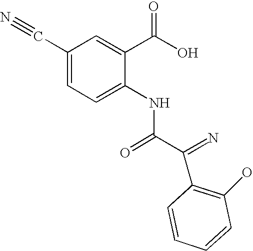

1,2-benzisoxazole-3-carboxylic acid (200 mg, 1.23 mmol) was suspended in CH₂Cl₂ (10 mL) and (COCI)₂ added (725 mg, 0.5 mL, 5.7 rmmol). A catalytic amount of DMF was then added and the mixture stirred for 4 hrs. The solvent was then removed in vacuo to give the acid chloride as an oil. The oil was dissolved in CHCl₃ (10 mL). tert-butyl 2-amino-5-cyanobenzoate (270 mg, 1.23 mmol) was added dropwise as a solution in THF/ pyridine (5 mL/1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by chromatography (1% MeOH in CHCl₃) to give 100 mg of the desired amide (22%). This amide was dissolved in 10 mL CH₂Cl₂/TFA (1/1) and stirred for 10 hrs at room temperature. The solvent was then removed in vacuo and the resulting solid was washed with MeOH, providing 80 mg of the title compound (95%). 1H NMR (400 MHz, DMSO) 7.59 (t, 1H), 7.81 (t, 1H), 7.96 (d, 1H), 8.16 (dd, 1H), 8.24 (d, 1H), 8.90 (d, 1H), 12.92 (s, 1H).

### Example 6.1: 2-[(1,2-benzisoxazol-3-ylcarbonyl)aminol-5-bromobenzoic acid

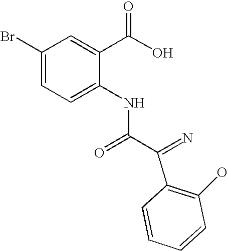

1,2-benzisoxazole-3-carboxylic acid (815 mg, 5.0 mmol) was suspended in dichloroethane (DCE) (10 mL). Oxalyl chloride (0.545 mL, 6.25 mmol) was added followed by a catalytic amount of DMF. The reaction was stirred at room temperature for 5 hrs. The solvent was removed in vacuo, and the resulting oil was redissolved in DCE (10 mL). Methyl 2-amino-5-bromobenzoate was added dropwise as a solution in THF/pyridine (5 mL/2 mL). The mixture was stirred for 48 hrs. The resulting solid was filtered and washed with MeOH, giving 925 mg (50%) of the desired amide. The ester was treated with LiOH in 1:1:1 THF/MeOH/H₂O for 12 hrs followed by acidification to give 608 mg (68%) of the title compound. 1HNMR (400 MHz, DMSO) 7.58 (t, 1H), 7.78 (t, 1H), 7.92 (dd, 1H), 7.95 (d, 1H), 8.16 (d, 1H), 8.24 (d, 1H), 8.71 (d, 1H), 12.60 (s, 1H).

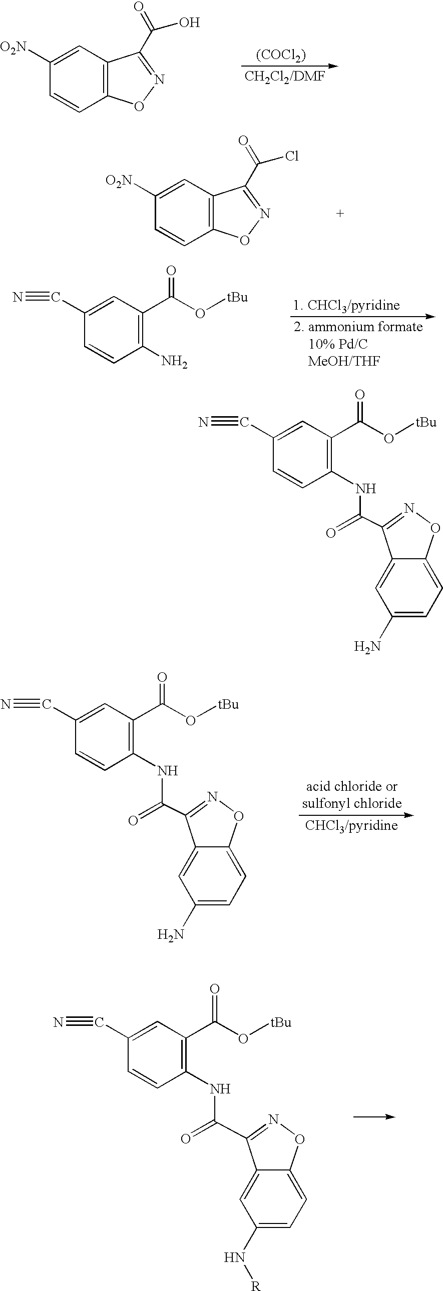

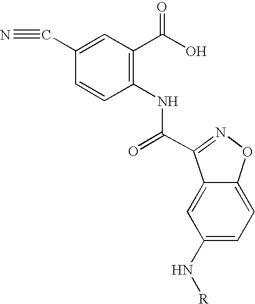

Preparation of tert-butyl 2-{[(5-amino-1,2-benzisoxazol-3-yl)carbonyl]amino}-5-cyanobenzoate

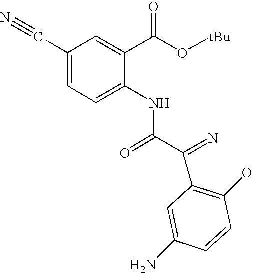

5-Nitro-1,2-benzisoxazole-3-carboxylic (2.08 g, 10 mmol) was suspended in CH₂Cl₂ (50 mL). Oxalyl chloride (1.1 ImL, 12.5 nmmol) was added followed by a catalytic amount of DMF. The reaction was stirred at room temperature for 5 hrs. The solvent was removed in vacuo, and the resulting residue was redissolved in CHCl₃ (50 mL). tert-Butyl 2-amino-5-cyanobenzoate was added dropwise as a solution in THF/pyridine (40 mL/10 mL). The mixture was stirred for 12 hrs at room temperature. The resulting solid was filtered and washed with MeOH, giving 2.9 g (72%) of the desired nitro-amide. The nitro-amide was reduced with 10% Pd/C in 100 mL (THF/MeOH, 1/1) with ammonium formate (4.7 g, 75 mmol) as H₂ source. After stirring for 10 hr at room temperature the mixture was filter and the resulting solids washed with THF. Excess solvent was removed in vacuo and the residue purified by chromatography (1% MeOH in CHCl₃) to give 2.61 g of the title compound (96%). 1H NMR (400 M/z, CDCl₃) 1.69 (s, 9H), 3.90 (bs, 2H), 7.04 (dd, 1H), 7.49 (m, 2H), 7.84 (dd, 1H), 8.37 (d, 1H), 9.06 (d, 1H), 12.76 (s, 1H).

### Example 6.2: 2-({[5-(acetylamino)-1,2-benzisoxazol-3-yl]carbonyl}amino)-5-cyanobenzoic acid

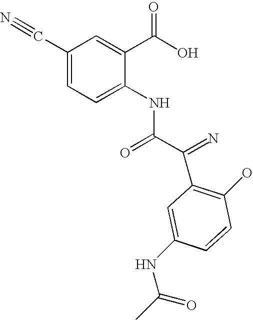

tert-Butyl 2-{[(5-amino-1,2-benzisoxazol-3-yl)carbonyl]amino}-5-cyanobenzoate (250 mg, 0.66 mmol) was dissolved in CHCl₃ (10 ml). Pyridine (1 mL) and acetyl chloride (57 μL, 0.8 mmol) were then added and the reaction stirred for 10 hrs at room temperature. The resulting mixture was absorbed onto silica and purified by chromatography (2% MeOH in CHCl₃) to give 203 mg (73%) of the desired acetamide. This amide was dissolved in 10 mL CH₂Cl₂/TFA (1/1) and stirred for 10 hrs at room temperature. Solvent was removed in vacuo and the remaining solid was recrystalized from THF/MeOH to give 170 mg (96%) of the title compound. 1H NMR(400 MHz, DMSO) 2.10 (s, 3H), 7.86 (m, 2H), 8.16 (dd, 1H), 8.45 (d, 1H), 8.63 (d, 1H), 8.91 (d, 1H), 10.32 (s, 1H), 12.86 (s, 1H).

### Example 6.3: 2-(I[5-(benzoylamino)-1,2-benzisoxazol-3-yl]carbonyl}amino)-5-cyanobenzoic acid

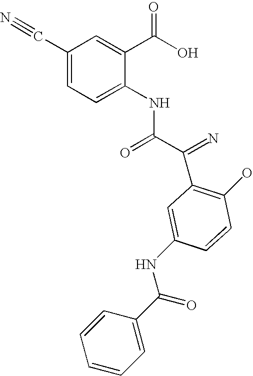

tert-Butyl 2-{[(5-amino-1,2-benzisoxazol-3-yl)carbonyl]amino}-5-cyanobenzoate (250 mg, 0.66 mmol) was dissolved in CHCl₃ (10 ml). Pyridine (1 mL) and benzoyl chloride (175 μL, 1.5 mmol) were then added and the reaction stirred for 10 hrs at room temperature. The resulting mixture was absorbed onto silica and purified by chromatography (2% MeOH in CHCl₃) to give 214 mg (67%) of the desired phenylamide. This amide was dissolved in 10 mL CH₂C₂I2TFA (1/1) and stirred for 10 hrs at room temperature. Solvent was removed in vacuo and the remaining solid was recrystalized from THF/MeOH to give 168 mg (88%) of the title compound. 1H NMR (400 MHz, DMSO) 7.60 (m, 3H), 7.95 (d, 1H), 8.02 (m, 2H), 8.12 (dd, 1H), 8.17 (dd, 1H) 8.45 (d, 1H), 8.81 (d, 1H), 8.92 (d, 1H), 10.62 (s, 1H), 12.92 (s, 1H).

### Example 6.4: 5-cyano-2-[({5-[(phenylacetyl)aminol-1,2-benzisoxazol-3-yl}carbonyl)amino]benzoic acid

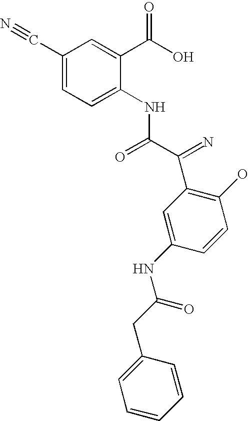

tert-Butyl 2-{[(5-amino-1,2-benzisoxazol-3-yl)carbonyl]amino}-5-cyanobenzoate (250 mg, 0.66 mmol) was dissolved in CHCl₃ (10 ml). Pyridine (1 mL) and phenylacetyl chloride (200 μL, 1.5 nmmol) were then added and the reaction stirred for 10 hrs at room temperature. The resulting mixture was absorbed onto silica and purified by chromatography (2% MeOH in CHCl₃) to give 151 mg (46%) of the desired phenylacetamide. This aride was dissolved in 10 mL CH₂Cl₂/TFA (1/1) and stirred for 10 hrs at room temperature. Solvent was removed in vacuo and the remaining solid was recrystalized from THF/MeOH to give 92 mg (68%) of the title compound. 1H NMR (400 MHz, DMSO) 3.70 (s, 2H), 7.30 (m, SH), 7.89 (m, 2H), 8.16 (dd, 1H), 8.44 (d, 1H) 8.63 (s, 1H), 8.89 (d, 1H), 10.57 (s, 1H), 12.89 (s, 1H).

### Example 6.5: 5-cyano-2-1({5-[(methylsulfonyl)aminol-1,2-benzisoxazol-3-yl}carbonyl) amino]benzoic acid

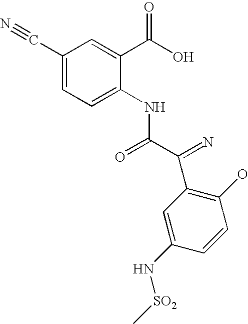

tert-Butyl 2-{[(5-amino-1,2-benzisoxazol-3-yl)carbonyl]amino}-5-cyanobenzoate (250 mg, 0.66 mmol) was dissolved in CHCl₃ (10 ml). Pyridine (1 mL) and methanesulfonyl chloride (116 μL, 1.5 mmol) were then added and the reaction stirred for 10 hrs at room temperature. The resulting mixture was absorbed onto silica and purified by chromatography (2% MeOH in CHCl₃) to give 234 mg (73%) of the desired methanesulfonamide. This amide was dissolved in 10 mL CH₂Cl₂/TFA (1/1) and stirred for 10 hrs at room temperature. Solvent was removed in vacuo and the remaining solid was recrystalized from THF/MeOH to give 194 mg (95%) of the title compound. 1H NMR (400 MHz, DMSO) 3.02 (s, 3H), 7.62 (dd, 1H), 7.95 (d, 1H), 8.08 (d, 1H), 8.16 (dd, 1H), 8.45 (d, 1H), 8.63 (d, 1H), 8.91 (d, 1H), 10.09 (s, 1H), 12.87 (s, 1H). MS (CI) m/z (rel. intensity) 418 (M+NH4, 100), 260 (7), 241 (9), 230 (10), 180 (33), 152 (19), 136 (87), 118 (64), 88 (19), 74 (43). Anal. Calcd for C₁₇ H₁₂ N₄ 06 S: C, 51.00; H, 3.02; N, 13.99;S, 8.01. Found: C, 49.27; H, 3.19; N, 13.38.

### Example 6.6: 5-cyano-2-[({5-[(phenylsulfonyl)amino]-1,2-benzisoxazol-3-yl}carbonyl)amino]benzoic acid

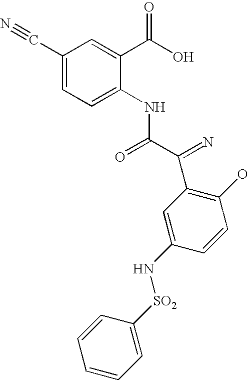

tert-Butyl 2-{[(5-amino-1,2-benzisoxazol-3-yl)carbonyl]amino}-5-cyanobenzoate (250 mg, 0.66 mmol) was dissolved in CHCl₃ (10 ml). Pyridine (1 mL) and benzenesulfonyl chloride (191 tL, 1.5 mmol) were then added and the reaction stirred for 10 hrs at room temperature. The resulting mixture was absorbed onto silica and purified by chromatography (2% MeOH in CHCl₃) to give 242 mg (67%) of the desired phenylamide. This amide was dissolved in 10 mL CH₂Cl₂/TFA (1/1) and stirred for 10 hrs at room temperature. Solvent was removed in vacuo and the remaining solid was recrystalized from THF/MeOH to give 201 mg (93%) of the title compound. 1H NMR (400 MHz, DMSO) 7.55 (m, 4H), 7.76 (m, 2H), 7.85 (d, 1H), 7.95 (d, 1H), 8.16 (dd, 1H) 8.43 (d, 111), 8.88 (d, 1H), 10.65 (s, 1H), 12.85 (s, 1H).

### Example 6.7: 2-[({5-[(benzylsulfonyl)aminol-1,2-benzisoxazol-3-yl}carbonyl)amino]-5-cyanobenzoic acid

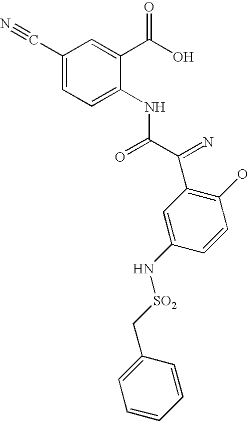

tert-Butyl 2-{[(5-amino-1,2-benzisoxazol-3-yl)carbonyl]amino}-5-cyanobenzoate (250 mg, 0.66 mmol) was dissolved in CHCl₃ (10 ml). Pyridine (1 mL) and α-toluenesulfonyl chilonue (200 μL, 1.5 mmol) were tnen added and tne reaction stirred for 10 hrs at room temperature. The resulting mixture was absorbed onto silica and purified by chromatography (2% MeOH in CHCl₃) to give 169 mg (48%) of the desired phenylacetamide. This amide was dissolved in 10 mL CH₂Cl₂/TFA (1/1) and stirred for 10 hrs at room temperature. Solvent was removed in vacuo and the remaining solid was recrystalized from THF/MeOH to give 102 mg (68%) of the title compound. 1H NMR (400 MHz, DMSO) 4.52 (s, 2H), 7.30 (m, 5H), 7.55 (dd, 1H), 7.92 (d, 1H), 8.07 (d, 1H), 8.16 (dd, 1H), 8.45 (d, 1H), 8.93 (d, 1H), 10.21 (s, 1H), 12.94 (s, 1H).

### Example 6.8: 2-{[(6-Chloro-1,2-benzisoxazol-3-yl)carbonyl]amino}-5-cyanobenzoic acid

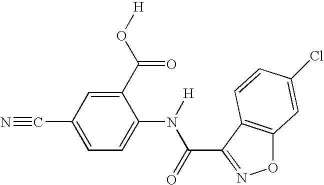

A solution of concentrated sulfuinc acid (15 mL) and water (5 mL) was added to a flask containing ethyl 6-chloro-1,2-benzisoxazole-3-carboxylate, as described in Chem. Pharm. Bull. 1998, 46, 84-96, (1.99 g, 8.82 iunol), and the resulting mixture was stirred in an 80° C. oil bath for 1 hour. A thick precipitate had formed which prevented stirring, so another 20 mL of the acid solution was added, and the mixture was stirred for an additional 2 hours at 80° C. The reaction mixture was then added to water. The resulting precipitate was washed with water and then dried briefly on a frit with a flow of air. It was then suspended in CH₂Cl₂ (60 mL) and treated with DMF (20 μL) and oxalyl chloride (1.4 mL, 16 mmol). An additional portion of oxalyl chloride (0.4 mL) was added after 35 minutes. After a further 40 minutes, the solvent and excess oxalyl chloride were removed by rotary evaporation. The residue was dissolved in CH₂Cl₂ (50 mL) and treated with tert-butyl 2-amino-5-cyanobenzoate (1.50 g, 6.87 rnmol) and pyridine (15 mL). The mixture was stirred overnight and then added to a separatory funnel with CH₂Cl₂ (150 mL). This solution was washed with 1.0 M HCl (2×100 mL) and brine (100 mL). Product was adsorbed onto silica gel, divided into two lots, and purified on Biotage Flash 40 M siliga gel cartridges using 70% CH₂Cl₂ in heptane as eluent. Product was collected as 2.46 g (70%) of white solid as the t-butyl ester. The corresponding tert-butyl ester (435 mg, 1.09 mmol) was stirred for 15.5 hours in a mixture of CH₂Cl₂ (30 mL) and TFA (20 mL). The solvents were removed by rotary evaporation, and the residue was recrystallized from ethanol/THF. The solids were washed with ethanol followed by heptane and then dried at 100° C. under vacuum yielding 250 mg of white solid. ¹H NMR (400 MHz, DMSO-D6) δ ppm 7.64 (dd, J=8.50, 1.66 Hz, 1H) 8.16 (dd, J=8.71, 2.07 Hz, 1H) 8.20 (d, J=8.71 Hz, 1H) 8.22 (d, J=1.66 Hz, 1H) 8.44 (d, J=2.07 Hz, 1H) 8.87 (d, J=8.92 Hz, 1H) 12.91 (s, 1H).

### Example 6.9: 5-Bromo-2-{[(6-chloro-1,2-benzisoxazol-3-yl)carbonyl]amino}benzoic acid

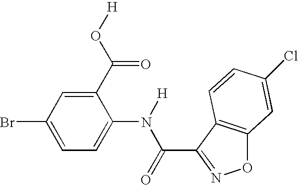

A solution of concentrated sulfuric acid (15 mL) and water (5 mL) was added to a flask containing ethyl 6-chloro-1,2-benzisoxazole-3-carboxylate (680 mg, 3.01 mmol), and the resulting mixture was stirred in an 80° C. oil bath for 3.3 hours. The reaction mixture was then added to water (50 mL). The resulting precipitate was washed with water and then dried briefly on a frit with a flow of air. It was then suspended in CH₂Cl₂ (40 mL) and treated with DMF (20 μL) and oxalyl chloride (0.50 mL, 5.7 mmol). Solvent and excess oxalyl chloride were removed by rotary evaporation after 50 minutes. The residue was dissolved in CH₂Cl₂ (30 mL) and treated with tert-butyl 2-amino-5-bromobenzoate (528 mg, 1.94 mmol) and pyridine (10 mL). The mixture was stirred overnight and then added to a separatory fumnel with CH₂Cl₂ (150 mL). This solution was aswhed with 1.0 M HC1 (2×100 μL) and brine (100 mL). Product was adsorbed onto silica gel and purified on a Biotage Flash 40 M simga gel cartridge using 40% CH₂Cl₂ in heptane as eluent. Product was collected as 690 mg (51%) of white solid as the t-butyl ester. The corresponding tert-butyl ester (497 mg, 1.10 mmol) was stirred for 6 hours in armixture of CH₂Cl₂ (15 mL) and TFA (15 mL). Solvents were removed by rotary evaporation, and the residue was recrystallized from THF. The solids were washed with THF followed by heptane and then dried at 100° C. under vacuum yielding 286 mg of white solid. ¹H NMR (400 MHz, DMSO-D6) δ ppm 7.63 (dd, J=8.50, 1.66 Hz, 1H) 7.91 (dd, J=9.02, 2.59 Hz, 1H) 8.15 (d, J=2.49 Hz, 1H) 8.20 (m, J=8.91 Hz, 2 H) 8.67 (d, J=8.91 Hz, 1H) 12.60 (s, 1H).

Preparation of (5-Bromo-2-fluorophenyl)(hydroxy)acetic acid

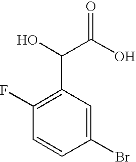

Trimethylsilyl cyanide (17.5 mL, 131 mmol, Aldrich) was added by syringe to a solution of 5-bromo-2-fluorobenzaldehyde (24.7 g, 122 mmol, Lancaster) and 1,4diazabicyclo[2.2.2]octa (1.0 g, 8.9 mmol, Aldrich) in CH₂Cl₂ (60 mL) at such a rate that the mixture maintained a moderate reflux. After 20 minutes of stirring, the mixture was diluted with CH₂Cl₂ (150 mL) and washed with water (3×150 mL) and brine (150 mL). The organics were dried over Na₂SO₄ and evaporated leaving the silylated cyanohydrin as 35 g of golden oil. This material was treated with concentrated HCl (75 mL) in water (25 mL) and then heated to reflux for 2 hours. The mixture was allowed to cool and then made basic with 6.0 M NaOH. The mixture was diluted with water (300 mL) and washed with CH₂Cl₂ (2×250 mL). It was then made acidic with concentrated HCl, and the product was extracted into CH₂Cl₂ (2×50 mL). The organics were dried over Na₂SO₄ and evaporated leaving the product as 21.5 g (71%) of white solid. ¹H NMR (400 MHz, DMSO-D6) δ ppm 5.22 (s, 1H) 6.18 (br s, 1H) 7.21 (dd, J=9.74, 8.91 Hz, 1H) 7.55 (ddd, J=8.71, 4.56, 2.70 Hz, 1 H) 7.62 (dd, J=6.32, 2.59 Hz, 1H) 12.92 (br s, 1H).

Preparation of Ethyl (5-bromo-2-fluorophenyl)(hydroxy)acetate

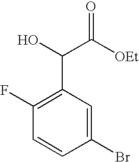

Sulfiurc acid (1.0 mL) was added to a solution of 5-bromo-2fluorophenyl)(hydroxy)acetic acid (20.0 g, 80.3 mmol) in ethanol (150 mL), and the solution was heated at reflux for 22 hours. The ethanol was removed by rotary evaporation, and the residue was dissolved in EtOAc. This solution was washed with saturated NaHCO₃ (2×200 μL) and brine (200 mL) and then dried over Na₂SO₄. Evaporation yielded the product as 20.6 g (93 %) of golden oil. ¹H NMR (400 MHz, ¹⁹F Decoupled, CHLOROFORM-D) δ ppm 1.24 (t, J=7.15 Hz, 3 H) 3.55 (d, J=4.98 Hz, 1H) 4.25 (m, 2 H) 5.35 (d, J=4.56 Hz, 1H) 6.97 (d, J=8.92 Hz, 1H) 7.43 (dd, J=8.71, 2.49 Hz, 1H) 7.53 (d, J=2.49 Hz, 1H).

Preparation of Ethyl (5-bromo-2-fluorophenyl)(oxo)acetate

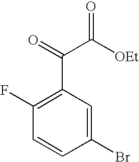

Acetic anhydride (8.0 μL, 85 mmol, Mallinckrodt) was added dropwise to a solution of ethyl (5-bromo-2-fluorophenyl)(hydroxy)acetate (19.1 g, 68.7 mmol) in DMSO (30 mL) with stirring in a 90° C. oil bath. The heat was removed after 3 hours. Water (10 μL) was added, and the mixture was stirred for 30 minutes before being added to a separatory finnel with EtOAc (100 μL). This rrixture was washed with brine (3×100 mL) and then dried over MgSO₄. Solvent was removed by rotary evaporation, and the residue was purified by chromatography on a Biotage Flash 75 L silica cartridge using 50 % CH₂Cl₂ in heptane as eluent. Yield was 14.0 g (74%) of white solid. ¹H NMR (400 MHz, ¹⁹F Decoupled, CHLOROFORM-D) δ ppm 1.40 (t, J=7.15 Hz, 3 H) 4.44 (q, J=7.26 Hz, 2 H) 7.08 (d, J=8.91 Hz, 1H) 7.73 (dd, J=8.81, 2.59 Hz, 1H) 8.03 (d, J=2.70 Hz, 1H).

Preparation of Ethyl (5-bromo-2-fluorophenyl)(hydroxyimino)ethanoate

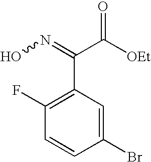

Hydroxylamine hydrochloride (3.57 g, 51.4 nmuol, Mallinckrodt) and sodium acetate (4.39 g, 53.5 mmol, Mallinckrodt) were added as solids to a solution of ethyl (5-bromo-2-fluorophenyl)(oxo)acetate (12.8 g, 46.4 mmol) in ethanol (30 μL). The mixture was stirred at room temperature for 3 hours and then at 50° C. for 1 hour. The mixture was allowed to cool and was then filtered. The precipitate was washed with ethanol, and these washings were added to the filtrate. The filtrate was concentrated to an oil. Water (200 mL) was added, and the product was extracted into EtOAc. The EtOAc was washed with an additional portion of water (100 mL) and then dried over MgSO₄. Evaporation left the product as 13.6 g (101%) of oil that was used without further purification. NMR and HPLC indicated a mixture of isomers. ¹H NMR (Major Isomer, 400 MHz, ¹⁹F Decoupled, DMSO-D6) δ ppm 1.24 (t, J=6.95 Hz, 3 H) 4.24 (q, J=7.05 Hz, 2 H) 7.29 (d, J=8.91 Hz, 1H) 7.61 (d, J=2.49 Hz, 1H) 7.68 (dd, J=8.81, 2.59 Hz, 1H) 12.98 (s, 1H).

Preparation of Ethyl 5-bromo-1,2-benzisoxazole-3-carboxylate

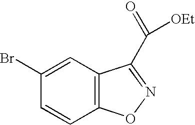

A mixture of ethyl (5-bromo-2-fluorophenyl)(hydroxyimino)ethanoate (2.15 g, 7.41 mmol) and potassium carbonate (1.13 g, 8.18 mmol, Mallinckrodt) in DMSO (10 mL) was heated in a 100° C. oil bath for 50 minutes. The mixture was diluted with MTBE (100 mL) and washed with water (2×100 mL). The organics were evaporated in the presence of silica gel, and the product was purified by chromatography using a Biotage Flash 40 M silica cartridge with 50 % CH₂Cl₂ in heptane as eluent. Yield was 1.44 g (72%) of white solid. ¹H NMR (400 MHz, DMSO-D6) 8 ppm 1.40 (t, J=7. 15 Hz, 3 H) 4.50 (q, J=7.12 Hz, 2 H) 7.94 (m, 2 H) 8.19 (m, 1H).

### Example 6.10: 2-{[(5-Bromo-1,2-benzisoxazol-3-yl)carbonyl]amino}-5-cyanobenzoic acid

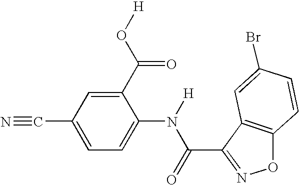

A solution of sulftiric acid (37.5 mL) in water (12.5 mL) was added to a flask containing ethyl 5-bromo-1,2-benzisoxazole-3-carboxylate (1.26 g, 4.67 mmol), and the mixture was heated in an 80° C. oil bath for 3.75 hours. The mixture was then added to 150 mL of water. The precipitate was collected, washed with water, and briefly air dried. The resulting white solid (711 mg) was sluried in CH₂Cl₂ (30 mL) and treated with DMF (20 μL) and oxalyl chloride (1.0 mL, 12 mmol). The solvent and excess oxalyl chloride were removed by rotary evaporation after 1.7 hours. The residue was dissolved in CH₂Cl₂ (20 mL) and treated with tert-butyl 2-amino-5-cyanobenzoate (547 mg, 2.51 mmol) and pyridine (8 mL). The mixture was stirred overnight and then added to a separatory funnel with 100 μL of EtOAc. A few mL of THF were added to improve solubility. This mixture was washed with 1.0 M HCl (2×100 mL) and brine (100 mL). Product was adsorbed onto silica gel and purified on a Biotage Flash 40 M siliga gel cartridge using a gradient from 50% CH₂Cl₂ to 75% CH₂Cl₂ in heptane as eluent. Product was collected as 647 mg (31%) of white solid as the t-butyl ester. tert-Butyl 2-{[(5-bromo-1,2-benzisoxazol-3-yl)carbonyl]amino}-5-cyanobenzoate (435 mg, 0.984 mmol) was stirred for 23 hours in a mixture of CH₂Cl₂ (10 mL) and TFA (10 mL). The solvents were removed by rotary evaporation, and the residue was recrystallized from THF/heptane. Yield was 89 mg of white solid. The mother liquor was concentrated, and the residue was recrystallized from EtOH/THF yielding an additional 162 mg of white solid. Total yield was 251 mg (66%). ¹H NMR (DMSO-d₆) δ 12.90 (s, 1H), 8.86 (d, J=8.9 Hz, 1H), 8.43 (d, J=2.1 Hz, 1H), 8.31-8.32 (m, 1H), 8.16 (dd, J=8.8, 2.2 Hz, 1H), 7.93-7.98 (m, 2 H).

### Example 6.11: 2-[(2,1-Benzisoxazol-3-ylcarbonyl)aminol-5-cyanobenzoic acid

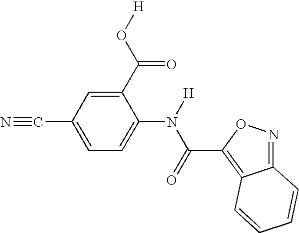

To a slurry of 2,1-benzisoxazole-3-carboxylic acid, as described in J. Chem. Soc. (C) 1970, 2660-2661, (1.01 g, 6.19 mmol) in CH₂Cl₂ (50 mL) was added DMF (20 μL) and oxalyl chloride (2.0 mL, 23 mmol). Solvent and excess oxalyl chloride were removed by rotary evaporation after 2.5 hours of stirring. The residue was dissolved in CH₂Cl₂ (30 μL), and tert-butyl 2-amino-5-cyanobenzoate (1.07 g, 4.90 mmol) in pyridine (20 mL) was added. After 3.5 hours, the mixture was diluted with CH₂Cl₂ (100 mL) and washed with 1 M HCl (2×100 μL) followed by brine (100 mmL). The product was adsorbed onto silica gel and purified on a Biotage Flash 40 M silica cartridge using a gradient from 60% to 75% CH₂Cl₂ in heptane as eluent. Product was corresponding tert-butyl ester (809 mg, 2.23 mmol) in CH₂Cl₂ (25 mL) was added TFA (15 mL). The solvents were removed by rotary evaporation after 23 hours. Methanol was added and then removed by rotary evaporation to remove residual TFA. The residue was then recrystallized from 50 mL of 4:1 ethanolYTHF. The collected solids were washed with methanol, CH₂Cl₂, and heptane and then dried at 100° C. under vacuum yielding 258 mg of pale yellow solid. ¹H NMR (400 MHz, DMSO-D6) δ ppm7.39 (dd,J=8.81, 6.32 Hz, 1H) 7.57 (dd,J=9.12, 6.43 Hz, 1H) 7.85 (d, 20 J=9.12 Hz, 1H) 8.03 (d, J=8.71 Hz, 1H) 8.15 (dd, J=8.71, 2.07 Hz, 1H) 8.44 (d, J=2.28 Hz, 1H) 8.86 (d, J=8.71 Hz, 1H) 12.95 (s, 1H).

Synthesis of 5- and 6- substituted 1,2-benzisoxazole-3-carboxylic acids

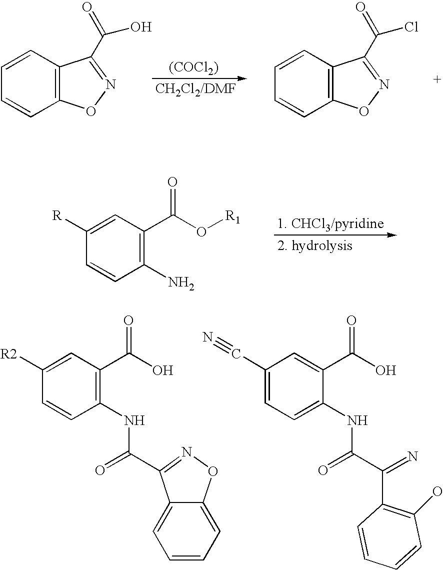

Such as described by in JACS 1975 97 7305.

### Example 6.13 2-[(1,2-benzisoxazol-3-ylcarbonyl)amino]-5-cyanobenzoic acid

1,2-benzisoxazole-3-carboxylic acid (200 mg, 1.23 mmol) was suspended in CH₂Cl₂ (10 mL) and (COCI)₂ added (725 mg, 0.5 mL, 5.7 mmol). A catalytic amount of DMF was then added and the mixture stirred for 4 hrs. The solvent was then removed in vacuo to give the acid chloride as an oil. The oil was dissolved in CHCl₃ (10 mL). tert-butyl 2-amino-5-cyanobenzoate (270 mg, 1.23 mmol) was added dropwise as a solution in THF/ pyridine (5 mLIl mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by chromatography (1% MeOH in CHCl₃) to give 100 mg of the desired amide (22%). This amide was dissolved in 10 mL CH₂Cl₂/TFA (1/1) and stirred for 10 hrs at room temperature. The solvent was then removed in vacuo and the resulting solid was washed with MeOH, providing 80 mg of the title compound (95%).

¹H NMR(400 MHz, DMSO) 7.59 (t, 1H), 7.81 (t, 1H), 7.96 (d, 1H), 8.16 (dd, 1H), 8.24 (d, 1H), 8.90 (d, 1H), 12.92 (s, 1H)

MS (EI) m/z (rel. intensity) 307 (M+, 0), 171 (81), 119 (59), 115 (46), 91 (99(30), 78 (31), 65 (26), 64 (59), 62 (46), 61 (68). HRMS (FAB) calcd for C₁₆H₉N₃O₄+H 308.0671, found 308.0665.

### Example 6.14: 2-[(1,2-benzisoxazol-3-ylcarbonyl)amino]-5-bromobenzoic acid

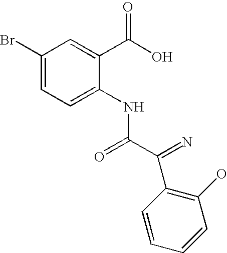

1,2-benzisoxazole-3-carboxylic acid (815 mg, 5.0 mmol) was suspended in dichloroethane (DCE) (10 mL). Oxalyl chloride (0.545 mL, 6.25 mmol) was added followed by a catalytic amount of DMF. The reaction was stirred at room temperature for 5 hrs. The solvent was removed in vacuo, and the resulting oil was redissolved in DCE (10 mL). Methyl 2-amino-5-bromobenzoate was added dropwise as a solution in THF/pyridine (5 mL/2 mL). The mixture was stirred for 48 hrs. The resulting solid was filtered and washed with MeOH, giving 925 mg (50%) of the desired amide. The ester was treated with LiOH in 1:1:1 THF/MeOH/H₂O for 12 hrs followed by acidification to give 608 mg (68%) of the title compound. H NMR (400 MHz, DMSO) 7.58 (t, 1H), 7.78 (t, 1H), 7.92 (dd, 1H), 7.95 (d, 1H), 8.16 (d, 1H), 8.24 (d, 1H), 8.71 (d, 1H), 12.60 (s, 1H)

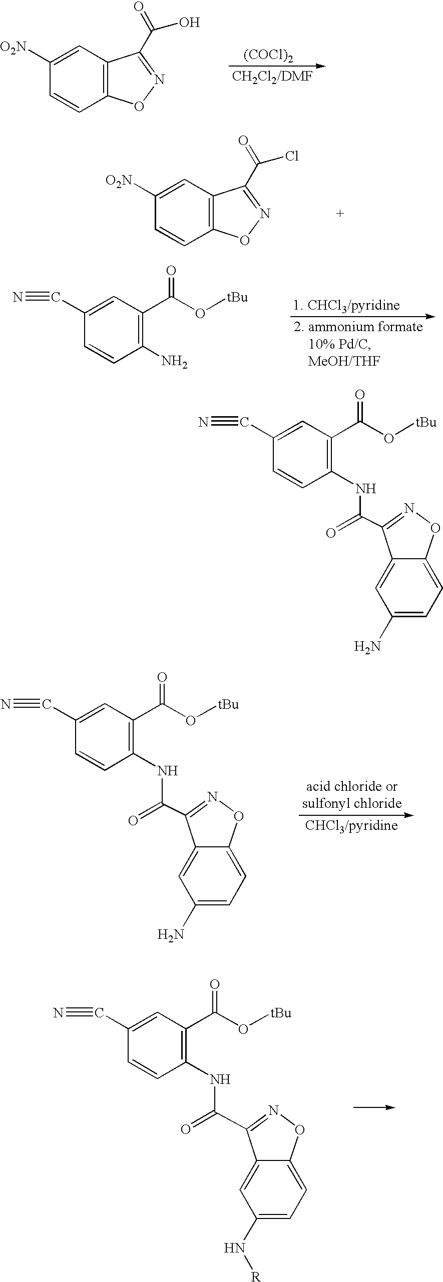

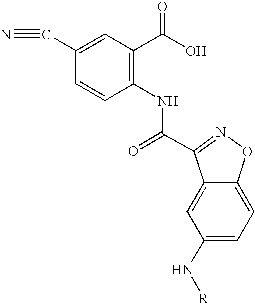

### Example 6.16: tert-butyl 2-{[(5-amino-1,2-benzisoxazol-3-yl)carbonyl]amino}-5-cyanobenzoate

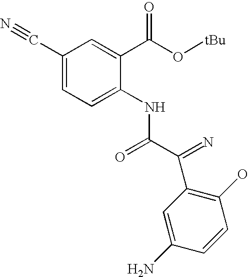

5-Nitro-1,2-benzisoxazole-3-carboxylic (2.08 g, 10 mmol) was suspended in CH₂Cl₂ (50 mL). Oxalyl chloride (1.1 mL, 12.5 mmol) was added followed by a catalytic amount of DMF. The reaction was stirred at room temperature for 5 hrs. The solvent was removed in vacuo, and the resulting residue was redissolved in CHCl₃ (50 mL). tert-Butyl 2-amino-5-cyanobenzoate was added dropwise as a solution in THF/pyridine (40 mL/10 mL). The mixture was stirred for 12 hrs at room temperature. The resulting solid was filtered and washed with MeOH, giving 2.9 g (72%) of the desired nitro-amide. The nitro-amide was reduced with 10% Pd/C in 100 mL (THF/MeOH, 1/1) with anmmonium formate (4.7 g, 75 mmol) as H₂ source. After stirring for 10 hr at room temperature the mixture was filter and the resulting solids washed with THF. Excess solvent was removed in vacuo and the residue purified by chromatography (1% MeOH in CHCl₃) to give 2.61 g of the title compound (96%). H NMR (400 MHz, CDCl₃) 1.69 (s, 9H), 3.90 (bs, 2H), 7.04 (dd, 1H), 7.49 (m, 2H), 7.84 (dd, 1H), 8.37 (d, 1H), 9.06 (d, 1H), 12.76 (s, 1H)

### Example 6.17: 2-({[5-(acetylamino)-1,2-benzisoxazol-3-yl]carbonyl}amino)-5-cyanobenzoic acid

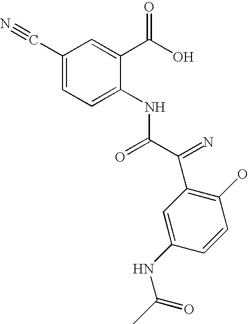

tert-Butyl 2-{[(5-amino-1,2-benzisoxazol-3-yl)carbonyl]amino}-5-cyanobenzoate (250 mg, 0.66 mmol) was dissolved in CHCl₃ (10 ml). Pyridine (1 mL) and acetyl chloride (57 μL, 0.8 mmol) were then added and the reaction stirred for 10 hrs at room temperature. The resulting mixture was absorbed onto silica and purified by chromatography (2% MeOH in CHCl₃) to give 203 mg (73%) of the desired acetamide. This amide was dissolved in 10 mL CH₂Cl₂/TFA (1/1) and stirred for 10 hrs at room temperature. Solvent was removed in vacuo and the remaining solid was recrystalized from THF/MeOH to give 170 mg (96%) of the title compound. H NMR (400 MHz, DMSO) 2.10 (s, 3H), 7.86 (m, 2H), 8.16 (dd, 1H), 8.45 (d, 1H), 8.63 (d, 1H), 8.91 (d, 1H), 10.32 (s, 1H), 12.86 (s, 1H)

### Example 6.18: 2-({[5-(benzoylamino)-1,2-benzisoxazol-3-yl]carbonyl}amino)-5-cyanobenzoic acid

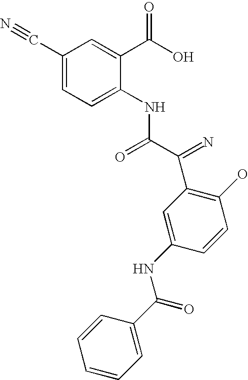

The title compound,168 mg, was synthesized as above from tert-butyl 2-{[(5-amino-1,2-benzisoxazol-3-yl)carbonyl]amino}-5-cyanobenzoate (250 mg, 0.66 mmol) and benzoyl chloride (175 ,L, 1.5 mmol). H NM (400 MHz, DMSO) 7.60 (m, 3H), 7.95 (d, 1H), 8.02 (m, 2H), 8.12 (dd, 1H), 8.17 (dd, 1H) 8.45 (d, 1H), 8.81 (d, 1H), 8.92 (d, 1H), 10.62 (s, 1H), 12.92 (s, 1H).

### Example 6.19: 5-cyano-2-[({5-[(phenylacetyl)amino]-1,2-benzisoxazol-3-yl]carbonyl)amino]benzoic acid

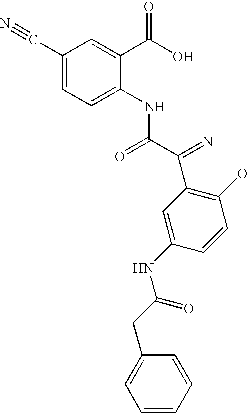

Synthesized as above fromtert-butyl 2-{[(5-amino-1,2-benzisoxazol-3-yl)carbonyl]amino}-5-cyanobenzoate (250 mg, 0.66 mmol) and phenylacetyl chloride (200 μL, 1.5 mmol) to give 92 mg of the title compound. H NMR (400 MHz, DMSO) 3.70 (s, 2H), 7.30 (m, 5H), 7.89 (m, 2H), 8.16 (dd, 1H), 8.44 (d, 1H) 8.63 (s, 1H), 8.89 (d, 1H), 10.57 (s, 1H), 12.89 (s, 1H).

### Example 6.20: 5-cyano-2-[({5-[(methylsulfonyl)amino]-1,2-benzisoxazol-3-yl}carbonyl) amino]benzoic acid

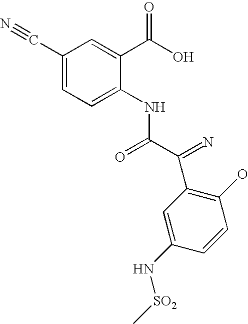

Synthesized as above from tert-butyl 2-{[(5-amino-1,2-benzisoxazol-3-yl)carbonyl]amino}-5-cyanobenzoate (250 mg, 0.66 immol) and methanesulfonyl chloride (116 μL, 1.5 mmol) to give 194 mg of the title compound. H NMR (400 MHz, DMSO) 3.02 (s, 3H), 7.62 (dd, 1H), 7.95 (d, 1H), 8.08 (d, 1H), 8.16 (dd, 1H), 8.45 (d, 1H), 8.63 (d, 1H), 8.91 (d, 1H), 10.09 (s, 1H), 12.87 (s, 1H).

### Example 6.21: 5-cyano-2-[({5-[(phenylsulfonyl)amino]-1,2-benzisoxazol-3-yl}carbonyl)amino]benzoic acid

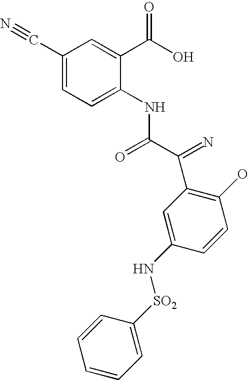

Synthesized as above from tert-butyl 2-{[(5-amino-1,2-benzisoxazol-3-yl)carbonyl]amino}-5-cyanoben (250 mg, 0.66 mmol) and benzenesulfonyl chloride (191 ,L, 1.5 mmol) to give 201 mg of the title compound. H NMR (400 MHz, DMSO) 7.55 (m, 4H), 7.76 (m, 2H), 7.85 (d, 1H), 7.95 (d, 1H), 8.16 (dd, 1H, 8.43 (d, 1H), 8.88 (d, 1H), 10.65 (s, 1H), 12.85 (s, 1H).

### Example 6.22: 2-[({5-[(benzylsulfonyl)amino]-1,2-benzisoxazol-3-yl}carbonyl)amino]-5-cyanobenzoic acid

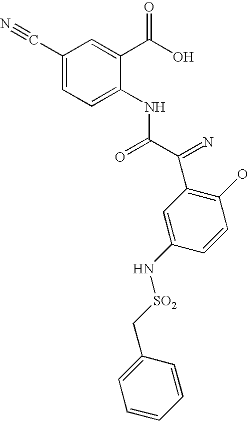

Synthesized as above from tert-butyl 2-{[(5-amino-1,2-benzisoxazol-3-yl)carbonyl]amino}-5-cyanobenzoate (250 mg, 0.66 mmol) and ac-toluenesulfonyl chloride (200 μL, 1.5 mmol) to give 102 mg of the title compound. H NMR (400 MHz, DMSO) 4.52 (s, 2H), 7.30 (m, 5H), 7.55 (dd, 1H), 7.92 (d, 1H), 8.07 (d, 1H), 8.16 (dd, 1H), 8.45 (d, 1H), 8.93 (d, 1H), 10.21 (s, 1H), 12.94 (s, 1H).

### Example 6.23: 5-cyano-2-{[(5-{[(4-fluorophenyl)sulfonyl]amino}-1,2-benzisoxazol-3-yl)carb acid

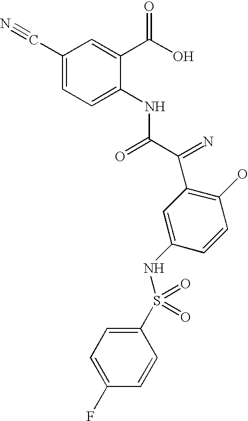

Synthesized as above from tert-butyl 2-{[(5-amino-1,2-benzisoxazol-3-yl)carbonyl]amino}-5-cyanobenzoate (250 mg, 0.66 immol) and 4fluorobenzenesulfonyl chloride (200 mg, 1.0 mmol) to give 205 mg of the title compound. H NMR (400 MHz, DMSO) 7.40 (m, 2H), 7.49 (dd, 1H), 7.80 (m, 2H), 7.87 (d, 1H), 7.94 (d, 1H), 8.16 (dd, 1H), 8.44 (d, 1H), 8.88 (d, 1H), 10.66 (s, 1H), 12.91 (s, 1H).

### Example 6.24: 5-cyano-2-[({5-[(methoxyacetyl)amino]-1,2-benzisoxazol-3-yl}carbonyl)amino]benzoic acid

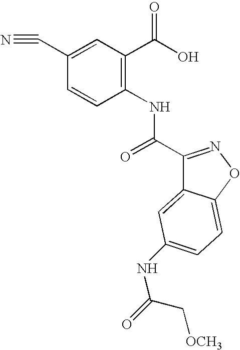

Synthesized as above from tert-butyl 2-{[(5-amino-1,2-benzisoxazol-3-yl)carbonyl]amino}-5-cyanobenzoate (150 mg, 0.44 mmol) and methoxyacetyl chloride (40 μL, 0.44 mmol) to give 106 mg of the title compound. H NMR (400 MHz, DMSO) 3.41 (s, 3H), 4.07 (s, 2H), 7.90 (d, 1H), 7.96 (dd, 1H), 8.17 (dd, 1H), 8.45 (d, 1H), 8.72 (d, 1H), 8.91 (d, 1H), 10.19 (s, 1H), 12.89 (s, 1H)

### Example 6.25: 5-cyano-2-[({5-[(cyclobutylcarbonyl)amino]-1,2-benzisoxazol-3-yl}carbonyl)amino]benzoic acid

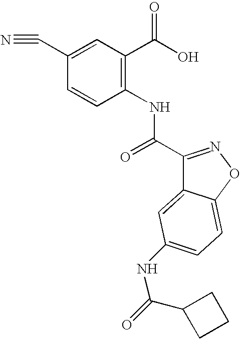

Synthesized as above from tert-butyl 2-{[(5-amino-1,2-benzisoxazol-3-yl)carbonyl]amino}-5-cyanobenzoate (150 mg, 0.44 mmol) and cyclobutylcarbonyl chloride (50 tL, 0.44 mmol) to give 112 mg of the title compound. H NMR (400 MHz, DMSO) 1.85 (m, 1H), 1.95 (m, 1H), 2.14 (m, 2H), 2.26 (m, 2H), 3.27 (m, 1H), 7.88 (d, 1H), 8.17 (dd, 1H), 8.45 (d, 1H), 8.66 (d, 1H), 8.90 (d, 1H), 10.11 (s, 1H), 12.89 (s, 1H).

### Example 6.26: 5-cyano-2-[({6-[(methylsulfonyl)amino]-1,2-benzisoxazol-3-yl}carbonyl) amino]benzoic acid

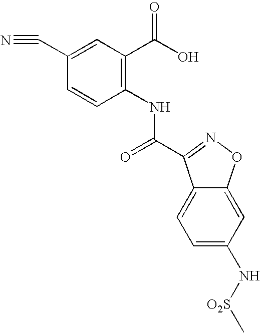

Synthesized as above from tert-butyl 2-{[(6-amino-1,2-benzisoxazol-3-yl)carbonyl]amino}-5-cyanobenzoat (250 mg, 0.66 mmol) and methanesulfonyl chloride (100 μL, 1.28 mmol) to give 220 mg of the title compound. H NMR (400 MHz, DMSO) 3.19 (s, 3H), 7.35 (dd, 1H), 7.65 (d, 1H), 8.14 (d, 1H), 8.16 (d, 1H), 8.45 (d, 1H), 8.89 (d, 1H), 10.59 (s, 1H), 12.87 (s, 1H)

### Example 6.27: 5-cyano-2-[({6-[(phenylsulfonyl)amino]-1,2-benzisoxazol-3-yl}carbonyl)amino]benzoic acid

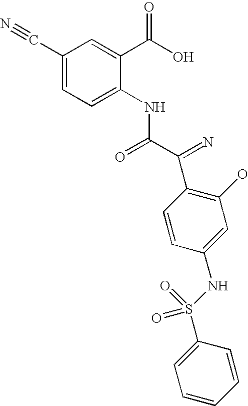

Synthesized as above from tert-butyl 2-{[(6-amino-1,2-benzisoxazol-3-yl)carbonyl]amino}-5-cyanobenzoate (250 mg, 0.66 mmol) and benzenesulfonyl chloride (150 μL, 1.17 rmmol) to give 252 mg of the title compound. H NMR (400 MHz, DMSO) 7.29 (dd, 1H), 7.55 (m, 4H), 7.88 (m, 2H), 8.05 (d, 1H), 8.15 (dd, 1H), 8.43 (d, 1H), 11.14 (s, 1H), 12.79 (s, 1H).

### Example 6.28: 5-cyano-2-{[(6-{[(4-fluorophenyl)sulfonyl]amino}-1,2-benzisoxazol-3-yl)carbonyl]amino}benzoic acid

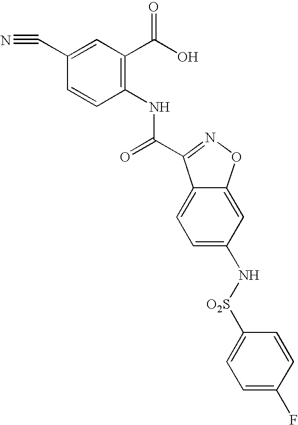

Synthesized as above from tert-butyl 2-{[(6-amino-1,2-benzisoxazol-3-yl)carbonyl]amino}-5-cyanobenzoate (250 mg, 0.66 mmol) and 4fluorobenzenesulfonyl chloride (200 mg, 1.0 mmol) to give 256 mg of the title compound. H NMR (400 MHz, DMSO) 7.29 (dd, 1H), 7.42 (m, 2H), 7.57 (d, 1H), 7.94 (m, 2H), 8.07 (d, 1H), 8.16 (dd, 1H), 8.43 (d, 1H), 8.85 (d, 1H), 11.15 (s, 1H), 12.80 (s, 1H).

### Example 6.29: 2-[({6-[(benzylsulfonyl)amino]-1,2-benzisoxazol-3yi}carbonyi)amino]-5-cyanobenzoic acid

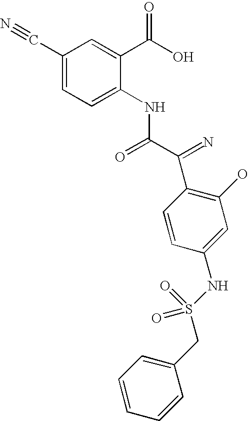

Synthesized as above from tert-butyl 2-{[(6-amino-1,2-benzisoxazol-3-yl)carbonyl]amino}-5-cyanobenzoate (250 mg, 0.66 mmol) and a-toluenesulfonyl chloride (200 mL, 1.5 mmol) to give 140 mg of the title compound. H NMR (400 MHz, DMSO) 5.17 (s, 2H), 7.00 (d, 1H) 7.50 (m, 5H), 7.78 (d, 1H), 8.16 (dd, 1H), 8.43 (d, 1H), 8.87 (d, 1H), 10.21 (s, 1H), 12.78 (s, 1H).

### Example 6.30: 2-{[(6-amino-1,2-benzisoxazol-3-yl)carbonyl]amino}-5-cyanobenzoic acid

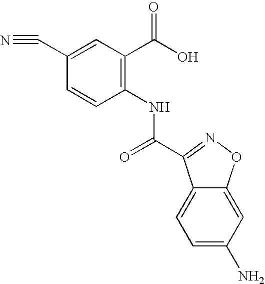

Synthesized as above from tert-butyl 2-{[(6-amino-1,2-benzisoxazol-3-yl)carbonyl]amino}-5-cyanobenzoate (250 mg, 0.66 mmol) to give 165 mg of the title compound. H NMR (400 MHz, DMSO) 6.73 (d, 1H), 6.80 (1H, dd), 7.78 (d, 1H), 8.14 (dd, 1i), 8.43 (d, 1), 8.87 (d, 1H), 12.70 (s, 1H).

### Example 6.31: 5-cyano-2-({[5-(ethylanrino)-1,2-benzisoxazol-3-yl]carbonyl}amino)benzoic acid

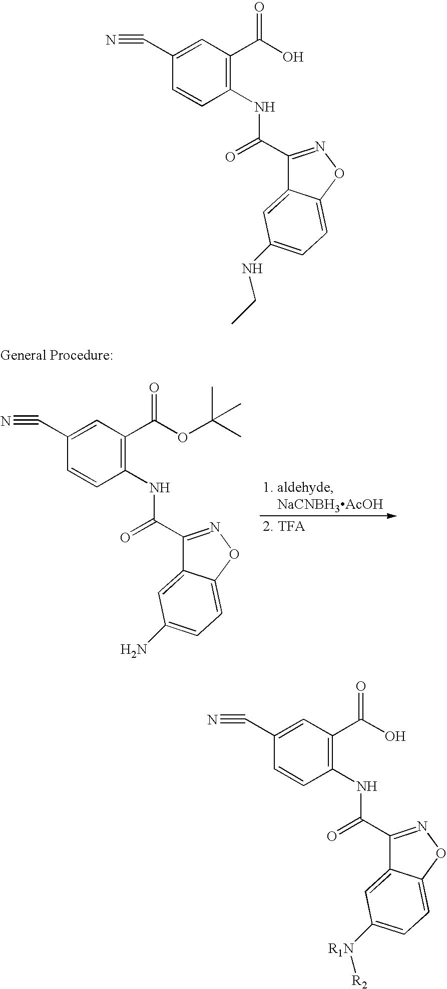

tert-Butyl 2-{[(5-amino-1,2-benzisoxazol-3-yl)carbonyl]amino}-5-cyanobenzoate (150 mg, 0.44 mmol) was suspended in 5 mL of THF/MeOH (1/1). To the suspension was added acetaldehyde (22 lL, 0.4 mmol), sodium cyanoborohydride (NaCNBH₃) (25 mg, 0.4 mmol) and 0.1 mL of acetic acid (AcOH). The reaction was monitored by HPLC and was diluted with 50 mL of CH₂Cl₂ when complete. The organic solution was washed with brine, dried over Na₂SO₄ and concentrated to a yellow solid. This solid was dissolved in 10 mL CH₂Cl₂/TFA (1/1) and stirred for 10 hrs at room temperature. Solvent was removed in vacuo and the remaining solid was washed with MeOH to give 60 mg of the title compound. H NMR (400 MHz, DMSO) 1.23 (t, 3H), 3.08 (q, 2H), 7.06 (d, 1H), 7.13 (dd, 1H), 7.65 (d, 1H), 8.13 (dd, l), 8.42 (d, 1H), 8.90 (d, 1H), 12.79 (s, 1H)

### Example 6.32: 5-cyano-2-[({5-[(cyclopropylmethyl)amino]-1,2-benzisoxazol-3-yl}carbonyl)amino]benzoic acid

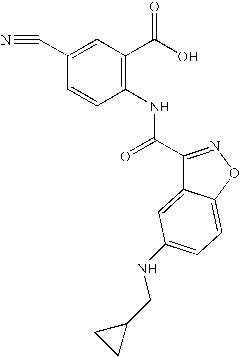

Synthesized as above from tert-butyl 2-{[(5-amino-1,2-benzisoxazol-3-yl)carbonyl]amino}-5-cyanobenzoate (150 mg, 0.44 mmol) and cyclopropyl carboxaldehyde (33 μL, 0.45 mmol) to give 58 mg of the title compound. H NMR (400 MHz, DMSO) 0.27 (m, 2H), 0.52 (m, 2H), 1.09 (m, 1H), 2.96 (d, 2H), 7.14 (s, 1H), 7.22 (d, 1H), 7.68 (d, 1H), 8.15 (dd, 1H), 8.44 (d, 1H), 8.91 (d, 1H), 12.80 (s, 1H).

### Example 6.33: 5-cyano-2-[({5-[(2-methoxyethyl)amino]-1,2-benzisoxazol-3-yl}carbonyl)amino]benzoic acid

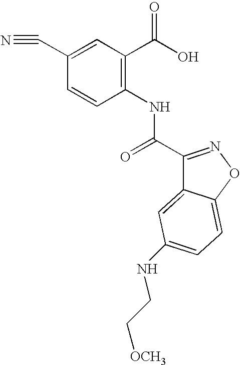

Synthesized as above from tert-butyl 2-{[(5-amino-1,2-benzisoxazol-3-yl)carbonyl]amino}-5-cyanobenzoate (150 mg, 0.44 mmol) and methoxyacetaldehyde (40 mg, 0.45 mmol) to give 75 mg of the title compound. H NMR (400 MHz, DMSO) 3.25 (t, 2H), 3.31 (s, 3H), 3.56 (t, 2H), 7.10 (d, 1H), 7.20 (dd, 1H), 7.66 (d, 1H), 8.15 (dd, 1H), 8.44 (d, 1H), 8.92 (d, I1H), 12.79 (s, 1H).

### Example 6.34: 5-cyano-2-[({5-[(2-hydroxyethyl)amino]-1,2-benzisoxazol-3-yl}carbonyl)amino]benzoic acid

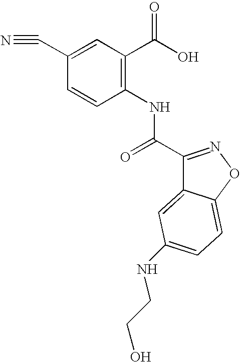

Synthesized as above from tert-butyl 2-{[(5-amino-1,2-benzisoxazol-3-yl)carbonyl]amino}-5-cyanobenzoate (150 mg, 0.44 mmol) and tertbutyldimethylsiloxyacetaldehyde (85 μL, 0.45 mmol) to give 111 mg of the title compound. HNMR(400 MHz, DMSO) 3.15 (t, 2H), 3.63 (t, 2H), 7.09 (d, 1H), 7.18 1o (dd, 1H), 7.65 (d, 1H), 8.15 (dd, 1H), 8.43 (d, 1H), 8.91 (d, 1H), 12.79 (s,

### Example 6.35: 5-cyano-2-[({5-[(2,3-dihydroxypropyl)amino]-1,2-benzisoxazol-3-yl}carbonyl)amin acid

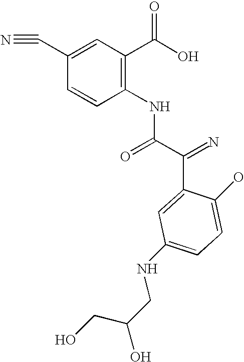

Synthesized as above from tert-butyl 2-{[(5-amino-1,2-benzisoxazol-3-yl)carbonyl]amino}-5-cyanobenzoate (150 mng, 0.44 mmol) and glyceraldehyde dimer (40 mg, 0.21 munol) to give 91 mg of the title compound. H NMR (400 MHz, 20 DMSO) 2.96 (dd, 1H), 3.22 (dd, 1H), 3.42 (m, 3H), 3.70 (m, 1H), 7.09 (d, 1H), 7.21 (dd, 1H), 7.65 (d, 1H), 8.15 (dd, 1H), 8.43 (d, 1H), 8.92 (d, I1H), 12.81 (s,

### Example 6.36a:

5-cyano-2-({[5-(dimethylamino)-1,2-benzisoxazol-3-yl]carbonyl}amino)benzoic acid

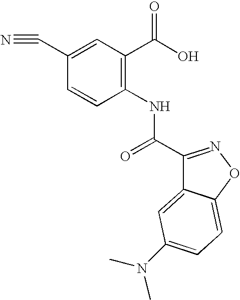

Synthesized as above from tert-butyl 2-{[(5-amino-1,2-benzisoxazol-3-yl)carbonyl]amino}-5-cyanobenzoate (150 mg, 0.44 mmol), formaldehyde (0.5 mL, 6.25 mmol) and NaCNBH₃ (95 mg, 1.5 mmol) to give 85 mg of the title compound. H NMR (400 MHz, DMSO) 2.99 (s, 6H), 7.24 (d, 1H), 7.35 (dd, 1H), 7.77 (d, 1H), 8.15 (dd, 1H), 8.43 (d, 1H), 8.91 (d, 1H), 12.84 (s, 1H).

### Example 6.36b: tert-butyl 2-({[6-(acetyloxy)-1,2-benzisoxazol-3-yl]carbonyl}amino)-5-cyanobenzoate

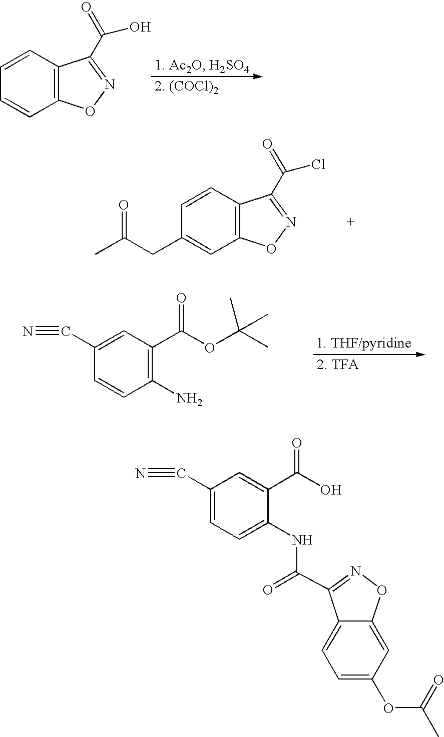

6-Hydroxy-1,2-benzisoxazole-3-carboxylic acid (4.5 g, 25 mmol) was dissolved in 25 mL of acetic anhydride. Several drops of concentrated sulfiric acid were added and the solution stirred at room temperature. After 4 h the reaction was poured onto ice and then extracted with CH₂Cl₂. The organic solution was dried over Na₂SO₄ and concentrated to give 6-acetoxy-1,2-benzisoxazole-3-carboxylic acid as white solid. This solid (2.8 g, 12.7 mmol) was suspended in 100 mL CH₂Cl₂ and 2.5 mL of oxalyl chloride was added followed by 10 drops of DMF. The reaction was stirred at room temperature for 3 h. The solution was concentrated to an off white solid and then redisolved in 100 mL CHCl₃. To this solution was added tert-butyl 2-amino-5-cyanobenzoate as a solution in THF/pyridine (40 mL/0 μL). After stirring for 12 h at room temperature the solution was concentrated to a pink solid. The solid was dissolved in EtOAc and washed with 1N HCl. The organic solution was dried over Na₂SO₄ and then concentrated to a white solid which was washed with MeOH to give 2.55 g of the title compound. H NMR (300 MHz, CDCl₃) 1.69 (s, 9H), 2.40 (s, 3H), 7.25 (dd, 1H), 7.53 (d, 1H), 7.86 (dd, 1H), 8.30 (d, 1H), 8.38 (d, 1H), 9.05 (d, 1H), 12.84 (s, 1H).

### Example 6.37: 2-({[6-(acetyloxy)-1,2-benzisoxazol-3-yl]carbonyl}amino)-5-cyanobenzoic acid

tert-Butyl 2-({[6-(acetyloxy)-1,2-benzisoxazol-3-yl]carbonyl}amino)-5-cyanobenzoate (250 mg, 0.59 nunol) was dissolved in 10 mL CH₂Cl₂/TFA (1/1) and stirred for 3 h at room temperature. Solvent was removed in vacuo and the remaining solid was washed with MeOH to give 206 mg of the title compound. H NMR (400 MHz, DMSO) 2.35 (s, 3H), 7.39 (dd, 1H), 7.85 (d, 1H), 8.16 (dd, 1H), 8.22 (d, 1H), 8.44 (d, 1H), 8.89 (d, 1H), 12.87 (s, 1H)

### Example 6.38a: tert-butyl

5-cyano-2-{[(6-hydroxy-1,2-benzisoxazol-3-yl)carbonyl]amino}benzoate

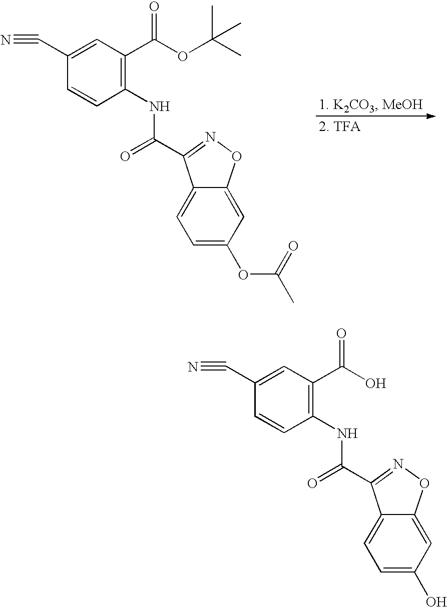

tert-Butyl 2-({[6-(acetyloxy)-1,2-benzisoxazol-3-yl]carbonyl}amino)-5-cyanobenzoate (1.93 g, 4.58 mmol) was suspended in 100 mL THF/MeOH (1/1). A solution of K₂CO₃ in 50 mL of H₂O and stirred for 3 h at room temperature. 50 μL of 1 N HCl was added and the resulting precipitate was filtered and dried overnight at 50 C in a vacuum oven to give 1.61 g of the title compound. H NMR (400 MHz, DMSO) 1.60 (s, 9H), 7.04 (dd, 1H), 7.14 (d, 1H), 7.97 (d, 1H), 8.16 (dd, 1H), 8.39 (d, 1H), 8.77 (d, 1H), 10.73 (s, 1H), 12.32 (s, 1H).

### Example 6.38b:

5-cyano-2-{[(6-hydroxy-1,2-benzisoxazol-3-yl)carbonyl]amino}benzoic acid

tert-Butyl

5-cyano-2-{[(6-hydroxy-1,2-benzisoxazol-3-yl)carbonyl]amino}benzoate (250 mg, 0.60 mmol) was dissolved in 10 mL CH₂Cl₂/TFA (1/1) and stirred for 4 h at room temperature. Solvent was removed in vacuo and the remaining solid was washed with MeOH to give 213 mg of the title compound. H NMR (400 MHz, DMSO) 7.03 (dd, 1H), 7.13 (d, 1H), 7.99 (d, 1H), 8.16 (dd, 1H), 8.44 (d, 1H), 8.89 (d, 1H), 10.73 (s, 1H), 12.87 (s, 1H)

### Example 6.39: 2-({[6-(benzyloxy)-1,2-benzisoxazol-3-yl]carbonyl})arino)-5-cyanobenzoic acid

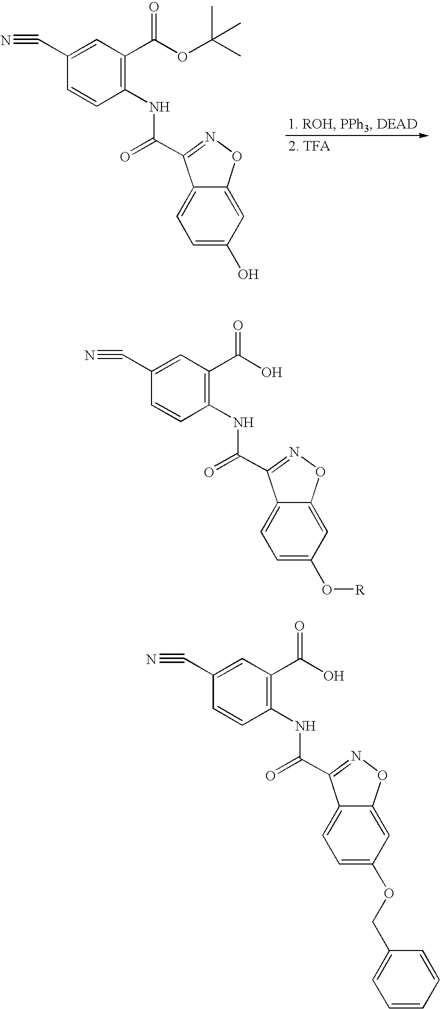

tert-Butyl

5-cyano-2-{[(6-hydroxy-1,2-benzisoxazol-3-yl)carbonyl]amino}benzoate (250 mg, 0.66 mmol) was dissolved in 10 mL of THF. To this solution was added benzyl alcohol (155 lL, 1.5 mmol), triphenyl phosphine (393 mg, 1.5 mmol) and diethylazodicarboxylate (236 μL, 1.5 mmol). After stirring at room temperature for 12 h the reaction was diluted with 50 mL of CH₂Cl₂ and filterd through SiO₂. The organic solution was concentrated to a oil and redisolved in 10 mL CH2C12/TFA (1/1) and stirred for 4 h at room temperature. Solvent was removed in vacuo and the remaining solid was washed with MeOH to give 35 mg of the title compound. H NMR (400 MHz, DMSO) 5.27 (s, 2H), 7.24 (dd, 1H), 7.37 (m, 1H), 7.43 (m, 2H), 7.51 (m, 2H), 7.61 (d, if), 8.06 (d, 1H), 8.15 (dd, 1H), 8.44 (d, 1H), 8.89 (d, 1H), 12.84 (s, 1H).

### Example 6.40: 5-cyano-2-{[(6-methoxy-1,2-benzisoxazol-3-yl)carbonyl]amino}benzoic acid

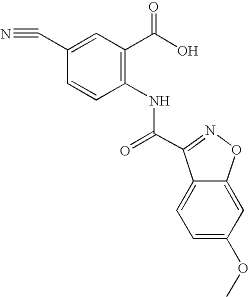

Synthesized as above from tert-butyl

5-cyano-2-{[(6-hydroxy-1,2-benzisoxazol-3-yl)carbonyl]amino}benzoate (250 mg, 0.66 mmol) and MeOH (60 [L, 1.5 mmol) to give 154 mg of the title compound. H NMR (400 MHz, DMSO) 3.91 (s, 3H), 7.17 (dd, 1H), 7.51 (d, 1H), 8.04 (d, 1H), 8.15 (dd, 1H), 8.44 (d, 1H), 8.89 (d, 1H), 12.81 (s, 1H).

### Example 6.41: 2-[({6-[(benzylsulfonyl)oxy]-1,2-benzisoxazol-3-yl}carbonyl)amino]

5-cyanobenzoic acid

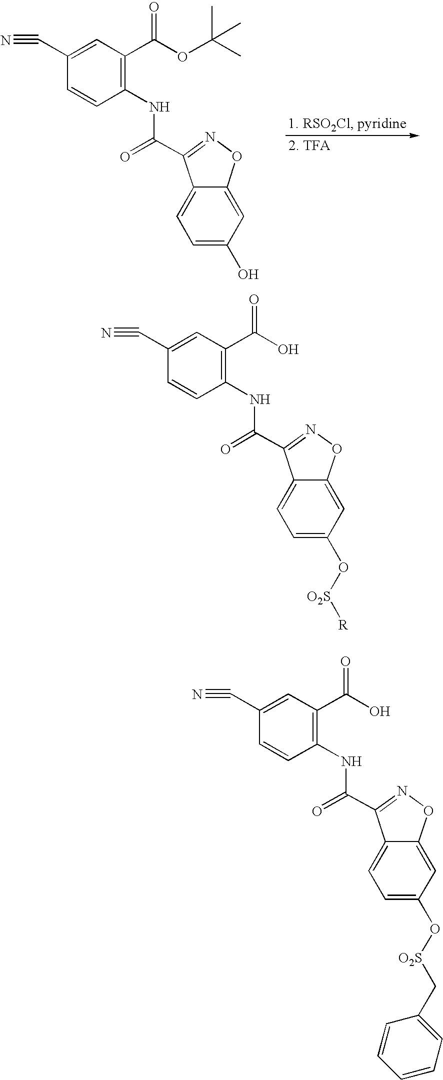

tert-Butyl

5-cyano-2-{[(6-hydroxy-1,2-benzisoxazol-3-yl)carbonyl]amino}benzoate (250 mg, 0.66 mmol) was dissolved in CHCl₃ (10 ml). Pyridine (1 mL) and α-toluenesulfonyl chloride (400 mg, 2.1 mmol) were then added and the reaction stirred for 12 h at 50 C. The solvent was removed in vacuo and the resulting solid washed with MeOH. This solid was dissolved in 10 mL CH₂Cl₂/TFA (1/1) and stirred for 10 hrs at room temperature. Solvent was removed in vacuo and the remaining solid was washed with MeOH to give 240 mg of the title compound. H NMR (400 MHz, DMSO) 5.14 (s, 2H), 7.45 (m, 3H), 7.54 (m, 2H), 7.92 (d, 1H), 8.17 (dd, 1H), 8.27 (d, 1H), 8.45 (d, 1H), 8.89 (d, 1H), 12.91 (s, 1H).

### Example 6.42: 5-cyano-2-[({6-[(phenylsulfonyl)oxy]-1,2-benzisoxazol-3-yl}carbonyl)amino]benzoic acid

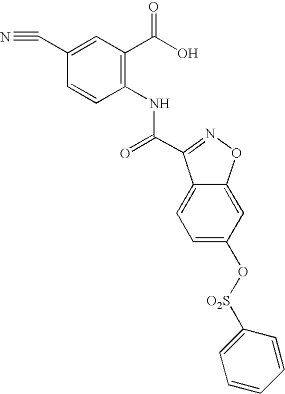

Synthesized as above from tert-butyl

5-cyano-2-{[(6-hydroxy-1,2-benzisoxazol-3-yl)carbonyl]amino}benzoate (250 mg, 0.66 mmol) and benzenesulfonyl chloride (400 μL, 3.1 mmol) to give 235 mg of the title compound. H NMR (400 MHz, DMSO) 7.24 (dd, 1H), 7.70 (t, 2H), 7.80 (d, 1H), 7.86 (t, 1H), 7.92 (m, 2H), 8.17 (dd, 1H), 8.19 (d, 1H), 8.45 (d, 1H), 8.85 (d, 1H), 12.86 (s, 1H).

### Example 6.43: Methyl 5-bromo-2-({[5-(chlorosulfonyl)-1,2-benzisoxazol-3-yl]carbonyl}amino)benzoate

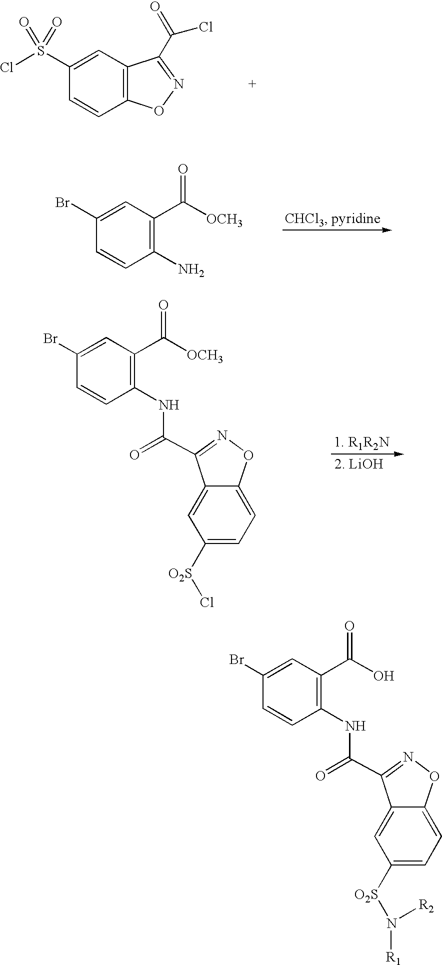

Ethyl-1,2-benzisoxazole-3-carboxylate (10 g, 52 rnmol) was dissolved in 100 mL of chlorosulfonic acid and heated to 80 C. After 16 h the reaction was cooled to room temperature, poured onto ice and then extracted with EtOAc. The organic solution was dried over Na₂SO₄ and then concentrated to a brown oil. This oil was dissolved in 60 mL of thionyl chloride and heated to 50 C. After 6 h excess reagent was removed in vacuo and the remaining residue dissolved in 300 mL CHCl₃. Methyl-2-amino-5-bromobenzoate was added as a solution in 100 mL CHCl₃ and 10 mL pyridine. After stirring overnight at room temperature the resulting precipitate was filtered providing 3.96 g of the title compound. H NMR (400 MHz, CDCl₃) 3.80 (s, 3H), 7.77 (dd, 1H), 7.92 (d, 1H), 8.27 (d, 1H), 8.32 (dd, 1H), 8.82 (d, 1H), 9.12 (d, lif), 12.63 (s, 1H)

### Example 6.44: 5-bromo-2-({[5-(morpholin-4-ylsulfonyl)-1,2-benzisoxazol-3-yl]carbonyl}amino)benzoic acid

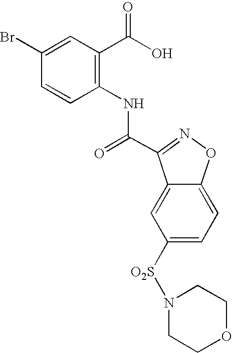

Methyl 5-bromo-2-({[5-(chlorosulfonyl)-1,2-benzisoxazol-3-yl]carbonyl}amino)benzoate (250 mg, 0.53 mmol) was suspended in 5 imL of THF. Morpholine (0.5 mL, 5.7 mmol) was added and the reaction warmed to 50 C. Solvent was removed in vacuo and the resulting solid washed with MeOH. This solid was suspended in 5 mL THF and 1 mL water and 50 mg LiOH was added. After 5 h the reaction was acidified with 1 N HCl and extracted with EtOAc. The organic solution was dried over Na₂SO₄ and concentrated to a white solid which was suspended in MeOH and filtered giving 140 mg of the title compound. H NMR (400 MHz, DMSO) 2.94 (m, 4H), 3.65 (m, 4H), 7.93 (dd, 1H), 8.11 (dd, 1H), 8.16 (d, 1H), 8.23 (d, 1H), 8.54 (d, 1H), 8.69 (d, 1H), 12.68 (s, 1H).

### Example 6.45: 2-({[5-(anilinosulfonyl)- i,2-benzisoxazol-3-yl]carbonyl}amino)-5-bromobenzoic acid

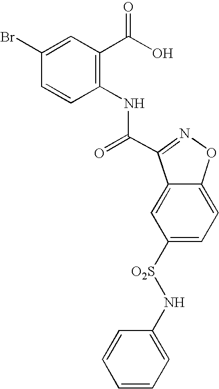

Synthesized as above from methyl 5-bromo-2-({[5-(chlorosulfonyl)-1,2-benzisoxazol-3-yl]carbonyl}amino)benzoate (250 mg, 0.53 mmol) and aniline (0.5 mL, 5.3 mmol) to give 99 mg of the title compound. H NMR (400 z, DMSO) 7.04 (t, I1H), 7.11 (d, 2H), 7.23 (2H), 7.93 (dd, 1H), 8.07 (dd, 1H), 8.12 (d, I1H), 8.16 (d, 1H), 8.64 (d, 1H), 8.67 (d, 1H), 10.52 (s, 1H), 12.68 (s, 1H).

### Example 6.46: 2-({[5-(dimethylaminosulfonyl)-1,2-benzisoxazol-3-yl]carbonyl}amino)-5-bromobenzoic acid

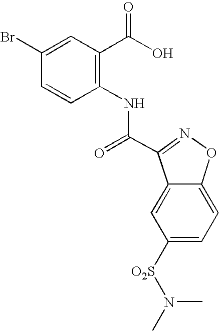

Synthesized as above from methyl 5-bromo-2-({[5-(chlorosulfonyl)-1,2-benzisoxazol-3-yl]carbonyl}amino)benzoate (250 mg, 0.53 mmol) and dimethylamine (1.0 mL, 2.0 mmol) to give 130 mg of the title compound. H NMR (400 MHz, DMSO) 2.67 (s, 6H) 7.93 (dd, 1H), 8.13 (dd, 1H), 8.16 (d, 1H), 8.21 (d, 1H), 8.54 (d, 1H), 8.69 (d, 1H), 12.67 (s, 1H).

### Example 6.47: 2-({[5-(diethylaminosulfonyl)-1,2-benzisoxazol-3-yl]carbonyl}amino)5-bromobenzoic acid

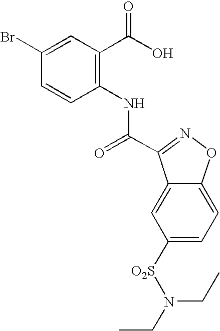

Synthesized as above from methyl 5-bromo-2-({[5-(chlorosulfonyl)-1,2-benzisoxazol-3-yl]carbonyl}amino)benzoate (250 mg, 0.53 mmol) and diethylamine (0.2 mL, 1.94 mmol) to give 150 mg of the title compound. H NMR (400 MHz, DMSO) 1.07 (t, 25 3H), 3.22 (q, 2H), 7.93 (dd, 1H), 8.16 (m, 3H), 8.58 (t, 1H), 8.69 (d, 1H), 12.66 (s, 1H).

### Example 6.48: 2-({[5-(benzylaminosulfonyl)-1,2-benzisoxazol-3-yl]carbonyl}amino)5-bromobenzoic acid

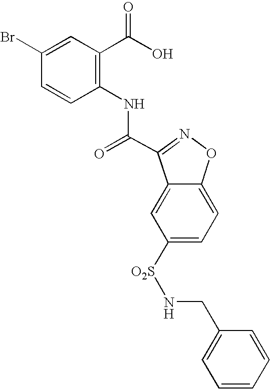

Synthesized as above from methyl 5-bromo-2-({[5-(chlorosulfonyl)-1,2-benzisoxazol-3-yl]carbonyl}amino)benzoate (250 mg, 0.53 mmol) and benzylamine (0.2 mL,1.83 mmol) to give 131 mg of the title compound. H NMR (400 MHz, DMSO) 4.04 (d, 2H), 7.19 (m, 5H), 7.93 (dd, 1H), 8.11 (d, 1H), 8.17 (d, 1H), 8.46 (t, 1H), 8.58 (t, 1H), 8.69 (d, 1H), 12.66 (s, 1H).

### Example 6.49: 2-({[5-(aminosulfonyl)-1,2-benzisoxazol-3-yl]carbonyl}amino)-5-bromobenzoic acid

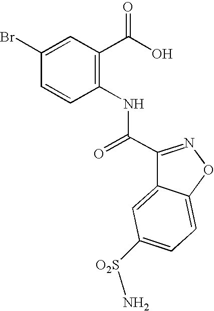

Synthesized as above from methyl 5-bromo-2-({[5-(chlorosulfonyl)-1,2-benzisoxazol-3-yl]carbonyl}amino)benzoate (250 mg, 0.53 mmol) and ammonia (4.0 mL, 2.0 mmol) to give 220 mg of the title compound. H NMR (400 MHz, DMSO) 7.63 (s, 2H), 7.93 (dd, 1H), 8.16 (m, 3H), 8.69 (dd, 1H), 12.67 (s, 1H).

### Example 6.50: 2-({[5-(methylaminosulfonyl)-1,2-benzisoxazol-3-yl]carbonyl}amino)5-bromobenzoic acid

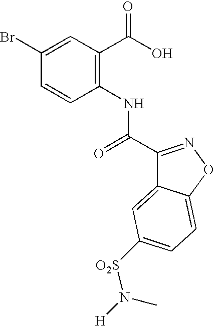

Synthesized as above from methyl 5-bromo-2-({[5-(chlorosulfonyl)-1,2-benzisoxazol-3-yl]carbonyl}amino)benzoate (250 mg, 0.53 mmol) and methylamine (5.0 mL, 10 mmol) to give 300 mg of the title compound. H NMR (400 MHz, DMSO) 2.45 (d, 3H), 7.75 (q, 1H), 7.93 (dd, 1H), 8.16 (m, 3H), 8.64 (d, 1H), 8.69 (d, 1H), 12.65 (s, 1H).

### Example 6.51: 2-({[5-(pyrrolidin-1-ylsulfonyl)-1,2-benzisoxazol-3-yl]carbonyl}amino)-5-bromobenzoic acid

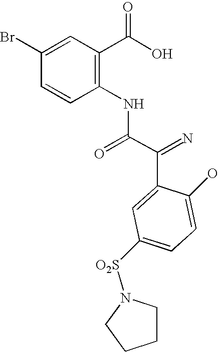

Synthesized as above from methyl 5-bromo-2-({[5-(chlorosulfonyl)-1,2-benzisoxazol-3-yl]carbonyl}amino)benzoate (250 mg, 0.53 mmol) and pyrrolodine (.125 mL, 1.5 mmol) to give 260 mg of the title compound. H NMR (400 MHz, DMSO) 1.67 (m, 4H) 3.20 (m, 4H), 7.93 (dd, 1H), 8.13 (dd, 1H), 8.16 (d, 1H), 8.19 (m, 2H), 8.58 (t, 1H), 8.69 (d, 1H), 12.67 (s, 1H).

### Example 6.52: 5-Cyano-2-({[6-(2-methylphenyl)-1,2-benzisoxazol-3-yl]carbonyl}amino)benzoic acid

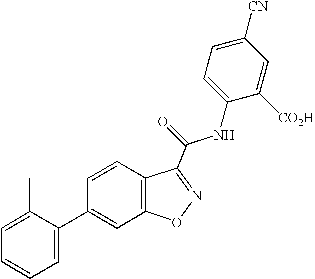

Preparation Pa: Ethyl 6(2-methylphenyl)benzoisoxazole-3-carboxylate.

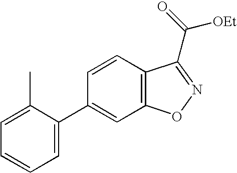

A mixture of ethyl 6-bromobenzoisoxazole-3-carboxylate (550 mg, 1.39 mmol), o-tolylboronic acid (377 mg, 1.5 eq), Pd(PPh₃)₄ (171 mg, 0.08 eq), toluene (10 mL), ethanol (1.5 mL), and 1 M Na₂CO₃ (1.5 mL) is stirred at reflux for 3.5 h. The reaction mixture is diluted with CH₂Cl₂ (0.1 L) and H₂O (0.1 mL). The CH₂Cl₂ solution is dried (Na₂SO₄), filtered, and evaporated. The residue is purified by silica chromatography (heptane/EtOAc) to give Preparation Pa: ¹H NMR (300 MHz, CDCl₃) δ 8.14 (1H), 7.61 (2H), 7.42 (1H), 7.35-7.2 (1H), 4.59 (2H), 2.28 (3H), 1.51 (3H).

A mixture of Preparation Pa (440 mg, 1.6 mmol) and

5-cyanoanthranilic acid (260 mg, 1.6 mmol) and 3 equiv of NaH (192 mg of 60% NaH in mineral oil) in DMF (3 mL) at rt under N₂ is stirred overnight. The reaction mixture is added dropwise to 0.1 L of aq 1 N HCl. The precipitated solid is collected and washed with water, heptane and iPrOH. It is recrystallized from EtOH to give a white solid: ¹H NMR (300 MHz, DMSO-d₆) 68.94 (1H), 8.45 (1H), 8.28 (11H), 8.18 (1H), 7.91 (1H), 7.56 (1H) 7.40-7.25 (4H) 2.24 (3H); MS (ESI−) n/z 396.22; Anal. C 69.19, H 3.85, N 10.64.

### Example 6.53: 5-Cyano-2-({16-(3,5-dimethylisoxazol-4-yl)-1,2-benzisoxazol-3-yl]carbonyl}amino)benzoic acid

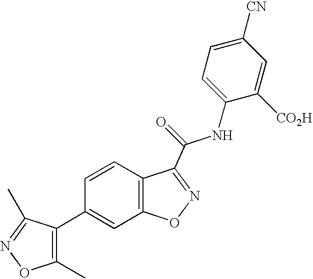

This example, prepared from ethyl 6-(3,5-dimethyl)-3-isoxazolecarboxylate and 1,1-dimethyl

5-cyanoanthranilate as described for Example 6.53, is isolated as a solid: ¹H NMR (300 MHz, DMSO-d₆) δ 8.94 (1H), 8.42 (1H), 8.32 (1H), 8.20 (1H), 8.05 (1H), 7.63 (1H), 2.30 (6H); MS (ESI−) n/z 401.14; H₂0 4.19% (0.98 equiv); Anal. C 59.89, H 3.86, N 13.27.

### Example 6.54: 5-Cyano-2-[({5-[4-(morpholin-4-ylcarbonyl)phenyl]-1,2benzisoxazol-3-yl}carbonyl)amino]benzoic acid

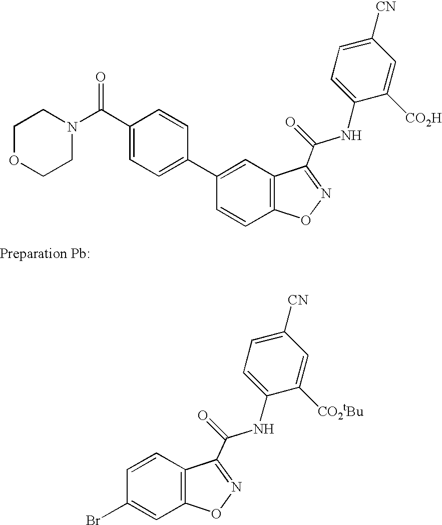

1,1-Dimethylethyl 2-[(6-bromobenzoisoxazole-3-carbonyl)aminol-5-cyanobenzoate. To a mixture of ethyl 6-bromobenzisoxazole-3-carboxylate (2.0 g, 7.4 mmol) and tbutyl

5-cyanoanthranilate (1.62 g, 7.4 mmol) in toluene (27 mL) is added NaH (1.5 g of a 60% mineral oil dispersion, 3.8 mmol) under N₂. The reaction mixture is stirred overnight at rt. This mixture is diluted with aq 1 N HCl, extracted with EtOAc (0.2 L). The EtOAc solution is dried and concentrated to give Preparation Pb as a solid: ¹H NMR (300 MHz, CDCl₃) δ 12.8 (1H), 9.05 (1H), 8.38 (1H), 8.18 (1H), 7.92 (1H), 7.82 (1H), 7.61 (1H), 1.69 (9H).

Preparation Pc:

1,1-Dimethylethyl 2-[(5-bromobenzoisoxazole-3-carbonyl)aminol-5-cyanobenzoate. This Preparation is made from ethyl 5-bromobenzisoxazole-3-carboxylate and t-butyl

5-cyanoanthranilate by the procedure given for Preparation Pb.

Preparation Pd:

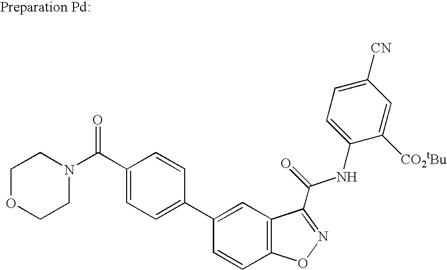

1,1-Dimethylethyl

5-cyano-2-({5-14-(morpholin-4-carbonyl)phenyl]benzo[dlisoxazole-3-carbonyl}A mixture of Preparation Pc (615 mg, 1.39 mmol), 4-(morpholin-4-ylcarbonyl)phenylboronic acid (536 mg, 1.5 equiv), Pd(PPh₃)₄ (118 mg, 0.08 equiv), toluene (15 μL), EtOH (2 mL), and 1 M aq Na₂CO₃ (2 μL) is stirred at reflux for 2.75 h. The reaction mixture is diluted with CH₂Cl₂. The reaction mixture is filtered through Celite. The CH₂Cl₂ solution is dried (Na₂SO₄), filtered, and evaporated. The residue is triturated with hot MeOH (50 mL). After cooling to rt a solid is collected by filtration. The solid is washed with MeOH and heptane, and dried to give Preparation Pd as an off-white solid: ¹H NMR (300 MHz, DMSO-d₆) δ 8.85 (1H), 8.43 (1H), 8.39 (1H), 8.11 (1H), 8.09 (1H), 8.02 (1H), 7.81 (2H), 7.57 (2H), 3.8-3.4 (8H), 1.65 (9H).

This Example, made by TFA deprotection of Preparation Pd is isolated as a solid: ¹H NMR(300 M[z, DMSO-d₆) δ 12.97 (1H), 8.94 (1H), 8.41 (1H), 8.10 (6H), 7.80 (1H), 7.53 (1H), 3.7 (8H); MS (CI) m/z (rel intensity) 497; Anal. C 65.33, H 4.04, N 11.28.

### Example 6.55: 2-({[5-(2-Acetylphenyl)-1,2-benzisoxazol-3-yl]carbonyl}amino)-5-cyanobenzoic acid

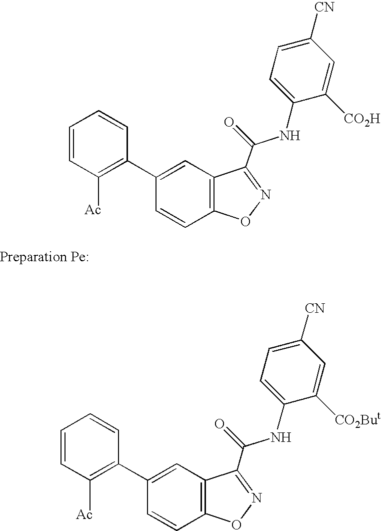

1,1-Dimethylethyl 2-{[5-(2-acetylphenyl)benzo[dlisoxazole-3-carbonyl]amino}-5-cyano-benzoate. This Preparation, made from Preparation Pc and 2acetylphenylboronic acid by the procedure given for Preparation Pd, is isolated as a solid: ¹H NMR (300 MHz, CDCl₃) 612.86 (1H), 9.04 (1H), 8.49 (1H), 8.39 (1H), 7.82 (1H), 7.72 (1H), 7.67 (1H), 7.4-7.6 (4H), 2.19 (3H), 1.69 (9H).

This Example, made by TFA deprotection of Preparation Pe is isolated as solid: ¹H NMR (300 MHz, DMSO-d₆) δ 12.85 (1H), 8.95 (1H), 8.41 (1H), 8.15 (1H), 8.07 (1H), 7.99 (1H), 7.6 (5H), 2.25 (3H); MS (ESI−) m/z 424.25 (calcd [M—H]-424.09); Anal. C 67.68, H 3.57, N 9.81.

### Example 6.56: 5-Cyano-2-({[5-(2,5-dimethoxyphenyl)-1,2-benzisoxazol-3-yl]carbonyl}amino)benzoic acid

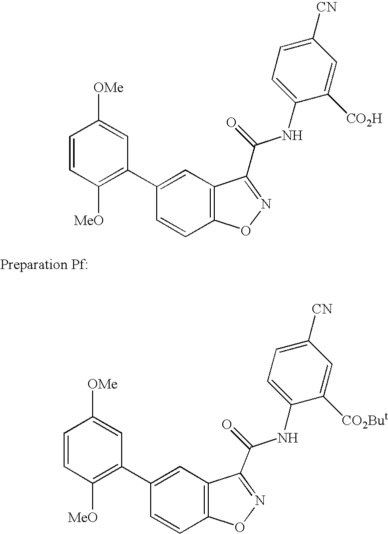

1,1-Dimethylethyl

5-cyano-2-{[5-(2,5-dimethoxyphenyl)benzo[dlisoxazole-3-carbonyl]-amino}benzoate This Preparation, made from Preparation Pc and 2,5-dimethoxyphenylboronic acid by the procedure given for Preparation Pd, is isolated as a solid: 1H NMR (300 MHz, CDCl₃) δ 12.83 (1H), 9.04 (1H), 8.51 (1H), 8.45 (1H) 7.8-7.9 (2H), 7.70 (1H), 6.85-7.0 (3H), 3.78 (3H), 3.82 (3H), 1.69

This Example, made by TFA deprotection of Preparation Pf is isolated as solid: ¹H NMR (300 MHz, DMSO-d₆) δ12.93 (1H), 8.92 (1H), 8.43 (1H), 8.35 (1H), 8.10 (1H), 7.95 (1H), 7.86 (1H), 7.10 (1H), 6.94 (1H), 3.75 (3H), 3.70 (3H); MS (ESI−) m/z 442.24; Anal. C 64.93, H 3.90, N 9.52.

### Example 6.57: 5-Cyano-2-({[5-(2-methylphenyl)-1,2-benzisoxazol-3-yl]carbonyl}amino)benzoic acid

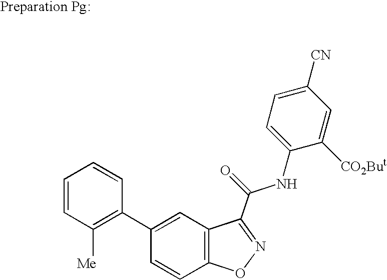

1,1-Dimethylethyl

5-cyano-2-{[5-(2-methylphenyl)benzoisoxazol-3-yl]carbonyl]amino}-benzoate. This Preparation, made from Preparation Pc and 2-methylphenylboronic acid by the procedure given for Preparation Pd, is isolated as a solid: ¹H NMR (300 MHz, CDCl₃) δ12.80 (1H), 9.03 (1H), 8.36 (1H), 8.24 (1H), 7.81 (1H), 7.72 (1H), 7.61 (1H), 7.2-7.35 (4H), 2.28 (3H), 1.66 (9H).

This Example, made by TFA deprotection of Preparation Pg is isolated as solid: MS (ESI−) m/z 396.23; MS (FAB+) m/z 397.1070; H₂O 1.98% (0.45 equiv); Anal. C 68.96, H 3.86, N 10.52.

### Example 6.58: 5-Cyano-2- [({5-[4-(methylsulfonyl)phenyl]-1,2-benzisoxazol-3-yl}carbonyl)amino]benzoic acid

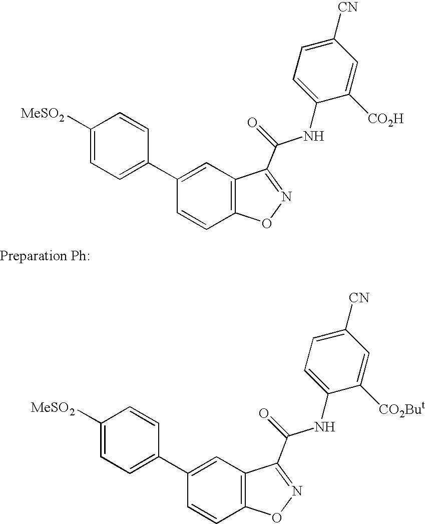

1,1-Dimethylethyl

5-cyano-2-{[5-(4-methylsuffonyl)phenyl]benzoisoxazol-3-yl}carbonyl]-amino}be This Preparation, made from Preparation Pc and 4-methylsulfonyl-phenylboronic acid by the procedure given for Preparation Pd, is isolated as a solid: ¹H NMR (300 MHz, DMSO-d₆) δ 8.82 (1H), 8.48 (1H), 8.40 (1H), 8.25-7.9 (7H), 3.32 (3H), 1.62 (9H).

This Example, made by TFA deprotection of Preparation Ph is isolated as solid: ¹H NMR (300 MHz, DMSO-d₆) δ 13.00 (1H), 8.92 (1H), 8.51 (1H), 8.45 (1H), 8.0-8.23 (7H), 3.32 (3H); MS (ESI−) m/z 460.24; MS (FAB+) m/z 461.0700; H₂O 1.12% (0.31 equiv); Anal. C 58.74, H 3.56, N 8.96.

### Example 6.59: 2-[({6-[3-(Acetylamino)phenyl]-1,2-benzisoxazol-3-yl}carbonyl)aminol-5-cyanobenzoic acid

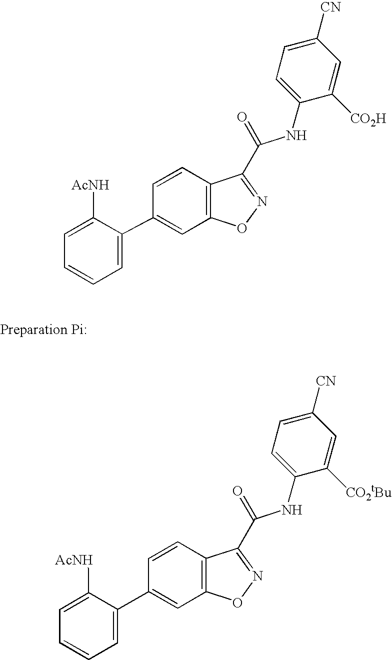

1,1-Dimethylethyl 2-{16-(2-acetylaminophenyl)benzoisoxazol-3-yl]carbonyl]amino}-5-cyanobenzoate. This Preparation, made from Preparation Pb and 2acetylamino-phenylboronic acid by the procedure given for Preparation Pd, is isolated as a solid: ¹HNMR(300 MHz, CDCl₃) δ12.82 (1H), 9.05 (1H), 8.35-8.4 (2H), 8.18 (1H), 7.84 (1H), 7.70 (1H), 7.55-7.40 (2H), 7.35-7.20 (2H), 7.05 (1H), 2.06 (3H), 1.70 (9H).

This Example, made by TFA deprotection of Preparation Pi is isolated as solid: ¹H NMR(300 MHz, DMSO-d₆) δ 9.25 (1H), 8.82 (1H), 8.31 (1H), 8.15 (1H), 8.00 (1H), 7.80 (1H), 7.5-7.2 (4H), 1.75 (3H); MS (ESI−) m/z 439.10.

### Example 6.60: 2-{[6-(3-Acetylaminophenyl)benzoisoxazole-3-carbonyl]amino}-5-cyano-benzoic acid.

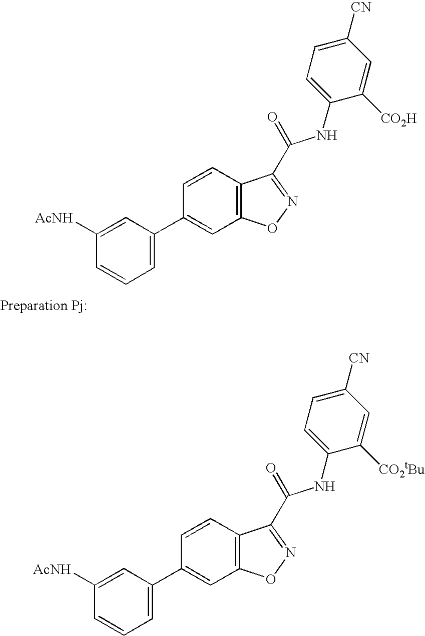

1,1-Dimethylethyl 2-{[6-(3-acetylaminophenyl)benzo [diisoxazole-3-carbonyl]amino}-5-cyanobenzo This Preparation, made from Preparation Pb and 3acetamido-phenylboronic acid by the procedure given for Preparation Pd, is isolated as a solid: 1HNMR(300 MHz, CDCl₃) δ12.9 (1H), 9.10 (1H), 8.38 (1H), 8.32 (2H), 7.95 (1H), 7.9-7.8 (2H), 7.72 (1H), 7.5-7.4 (3H), 2.25 (3H), 1.72 (9H).

This Example, made by TFA deprotection of Preparation Pj is isolated as solid: ¹H NMR(300 MHz, DMSO-d₆) δ 9.95 (1H), 8.78 (1H), 8.31 (1H), 8.15 (1H), 8.05 (2H), 7.90 (1H), 7.70 (1H), 7.54 (1H), 7.4-7.3 (2H), 1.93 (3H); MS (ESI−) m/z 439.09.

### Example 6.61: 2-[({5-12-(Acetylamino)phenyl]-1,2-benzisoxazol-3-yl}carbonyl)amino]-5-cyanobenzoic acid

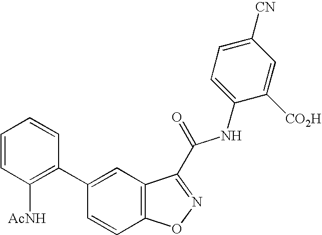

Preparation Pk:

1,1-Dimethylethyl 2-[({5-[2-(acetylamino)phenyl]- I,2-benzisoxazol-3-yl}carbonyl)amino]-5-cyanobenzoate. This Preparation, made from Preparation Pc and 2-acetylaminophenylboronic acid by the procedure as given for Preparation Pd, is isolated as a solid.

This Example, made by TFA deprotection of Preparation Pk is isolated as solid: ¹H NMR (300 MHz, DMSO-d₆) δ12.89 (1H), 9.36 (1H), 8.87 (1H), 8.41 (1H), 8.15 (1H), 8.11 (1H), 7.97 (1H), 7.72 (1H), 7.5-7.25 (4H), 1.9 (3H).

### Example 6.62: 2-[({5-[3-(Acetylamino)phenyl]-1,2-benzisoxazol-3-yl}carbonyl)aminol-5-cyanobenzoic acid

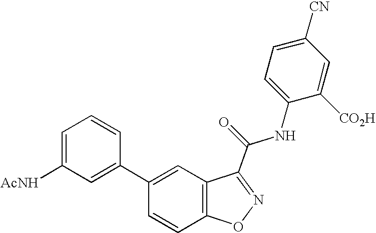

Preparation Pl:

1,1-Dimethylethyl 2-[({5-[3-(acetylamino)phenyl]-1,2-benzisoxazol-3-yl}carbonyl)amino]-5-cyanobenzoate. This Preparation, made from Preparation Pc and 3-acetylaminophenylboronic acid by the procedure given for Preparation Pd, is isolated as a solid.

This Example, made by TFA deprotection of Preparation PI is isolated as solid: ¹H NMR (300 MHz, DMSO-d₆) δ12.95 (1H), 10.12 (1H), 8.91 (1H), 8.49 (1H), 8.39 (1H), 8.19 (1H), 8.1-7.9 (2H), 7.67 (1H), 7.72 (1H), 7.5-7.4 (2H), 2.08 (3H).

### Example 6.63: Benzyl

5-cyano-2-({[5-(pyrrolidin-1-ylsulfonyl)-1,2-benzisoxazol-3-yl]carbonyl}

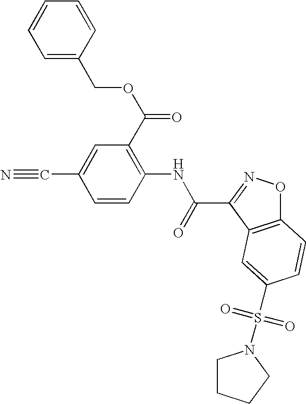

Preparation of 5-(Chlorosulfonyl)1,2-benzisoxazole-3-carboxylic acid

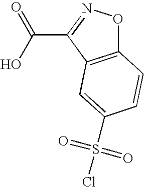

Ethyl 1,2-benzisoxazole-3-carboxylate (586 mg, 3.1 mmol) was dissolved in chlorosulfonic acid (10 mL, Aldrich) under N₂ and heated to 80° C. for 15 hours. The dark brown solution was carefully poured into a flask with crushed ice. The resultant tan precipitate was collected by vacuum filtration and washed thoroughly with water. Air-drying yielded 382 mg (49%) of a tan solid. ¹H NMR (400 MHz, DMSO-d₆) δ ppm 7.83 (d, J=8.71 Hz, 1H) 7.96 (dd, J=8.71, 1.66 Hz, 1H) 8.32 (d, J=1.66 Hz, 1 H) 14.23 (s, 1H); ¹³C NMR (100 MHz, DMSO-d₆) δ ppm 109.61, 118.89, 119.99, 129.17, 145.98, 151.49, 160.70, 163.42.

### Preparation of tert-Butyl 2-({[5-(chlorosulfonyl)-1,2-benzisoxazol-3-yl]carbonyl}amino)-5-cyanobenzoate

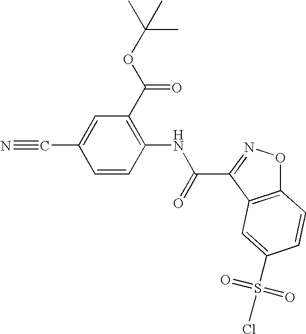

5-(Chlorosulfonyl)-1,2-benzisoxazole-3-carboxylic acid (346 mg, 1.32 mmol) was dissolved in dry CH₂Cl₂ (10 mL) under N₂ and treated with DMF (50 μL) followed by oxalyl chloride (0.231 mL, 2.64 mmol). Gas was evolved, and the mixture was stirred for 1 hour. The solvent and excess oxalyl chloride were evaporated, and the residue was combined with tert-butyl 2-amino-5-cyanobenzoate (218 mg, 1.00 mmol) in 15 mL of dry CH₂Cl₂. The solution was refluxed for 30 minutes and then diluted to 100 mL with CH₂Cl₂. The organic solution was washed with 1.0 M HCl, with water, and with brine (75 mL each). The organic layer was dried over Na₂SO₄, filtered, and evaporated. The crude product was purified on a Biotage Flash 40M+silica cartridge using 95% CH₂Cl₂ in heptane. Evaporation of solvent from the product fractions followed by drying under high vacuum afforded 162 mg (35%) of an off-white solid. ¹H NMR (400 MHz, CDCl₃) δ ppm 1.68 (s, 9 H) 7.88 (dd, J=8.81, 1.97 Hz, 1H) 7.94 (d, J=9.12 Hz, 1H) 8.34 (dd, J=9.02,1.97 Hz, 1H) 8.38 (d, J=1.87 Hz, 1H) 9.03 (d, J=8.91 Hz, 1H) 9.10 (d, J=1.87 Hz, 1H) 12.98 (s, 1H); ³C NMR (100 MHz, CDCl₃) 8ppm28.13, 84.80, 107.30, 111.88, 117.90, 118.09, 120.49, 121.24, 125.27, 129.02,135.70, 137.15, 141.75, 143.39, 152.76, 156.86, 165.75, 166.19.

### Preparation of Benzyl 2-({[5-(chlorosulfonyl)-1,2-benzisoxazol-3-yl]carbonyl}amino)-5-cyanobenzoate

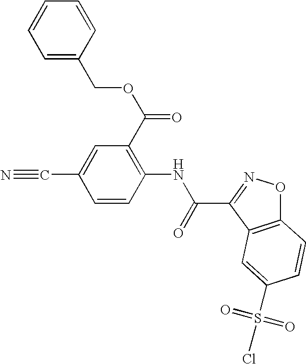

5-(Chlorosulfonyl)-1,2-benzisoxazole-3-carboxylic acid (1.25 g, 4.78 mmol) was dissolved in dry CH₂Cl₂ (15 mL) under N₂ and treated with DMF (50 μL) followed by oxalyl chloride (0.83 mL, 9.56 mmol). Gas was evolved, and the mixture was stirred for 1 hour. The solvent and excess oxalyl chloride were evaporated, and the residue was combined with benzyl 2-amino-5-cyanobenzoate (1.13 g, 4.48 mmol) in 15 mL of dry CH₂Cl2. The solution was refluxed for 20 minutes and then stirred at room temperature overnight. The reaction mixture was diluted with CH₂Cl₂ and filtered through a plug of silica. Evaporation of the solvent followed by drying under high vacuum afforded 1.87 g (84%) of a pale yellow solid. ¹H NMR (400 MHz, CDCl₃) δ ppm 5.49 (s, 2 H) 7.46 (m, 5 H) 7.91 (dd, J=8.81, 1.97 Hz, 1H) 7.95 (d, J=8.91 Hz, 1 H) 8.34 (dd, J=9.02,1.97 Hz, 1H) 8.47 (d, J=1.87 Hz, 1H) 9.06 (d, J=8.71 Hz, 1H) 9.10(d,J=1.45 Hz, 1H) 12.90(s, 1H). ¹³CNMR(100 MHz, DMSO-d₆) ppm 67.47, 106.41, 109.81, 117.61, 117.88, 118.47, 119.99, 121.46, 128.31, 128.38, 128.55, 129.60, 135.14, 135.22,138.02,142.73, 146.32,152.22,157.54, 163.71, 165.72.

Benzyl 2-({[5-(chlorosulfonyl)-1,2-benzisoxazol-3-yl]carbonyl}amino)-5-cyanobenzoate (410 mg, 0.83 immol) was dissolved in dry CH₂Cl₂ (10 mL) and treated with pyrrolidine (0.145 mL, 1.74 immol, Aldrich) resulting in a cream colored precipitate. After 15 minutes, the reaction mixture was diluted to 200 mL with CH₂Cl₂ and washed 2× with 1.0 M HCl and 1× with brine (175 mL each). The golden organic solution was dried over Na₂SO₄ and filtered. Evaporation of the solvent followed by drying under high vacuum afforded 432 mg (98%) of white solid. ¹H NMR (400 MHz, DMSO-d₆) δ ppm 2.50 (m, 4 H) 3.20 (m, 4 H) 5.45 (s, 2 H) 7.38 (m, 3 H) 7.52 (d, J=6.63 Hz, 2 H) 8.21 (m, 3 H) 8.49 (d, J=1.87 Hz, 1H) 8.56 (s, 1 H) 8.76 (d, J=8.92 Hz, 1H) 12.43 (s, 1H). ¹³C NMR (100 MHz, DMSO-d₆ CDCl₃) Δ ppm 24.64, 47.51,67.52,106.62,110.76, 115.93, 117.09, 119.54, 120.75, 123.48, 128.00, 128.23, 128.28, 129.20, 134.02,134.27, 134.87, 137.21, 142.96, 151.81, 156.69, 165.01, 165.56. IR (diffuse reflectance) 2498 (w), 2470 (w), 2395 (w), 2351 (w), 2230, 1709 (s), 1583 (s), 1519 (s), 1348 (s), 1316, 1294, 1268 (s), 1236 (s), 1199, 741, cm⁻¹. Anal. Calc. for C₂₇H₂₂N_{4O}₆S: C, 61.12; H, 4.18; N, 10.56;S, 6.04. Found: C, 61.00; H, 4.31; N, 10.54.

### Example 6.64: 5-Cyano-2-({[5-(pyrrolidin-1-ylsulfonyl)-1,2-benzisoxazol-3-yl]carbonyl}amino)benzoic acid

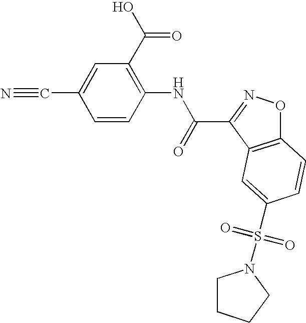

To a mixture of benzyl

5-cyano-2-({[5-(pyrrolidin-1-ylsulfonyl)-1,2-benzisoxazol-3-yl]carbonyl}ami (336 mg, 0.63 mmol) and 10% palladium on carbon (84 mg, Aldrich) was added dry, inhibitor-free THF (15 mL). The mixture was placed under 1 ATM of H₂, and the reaction was stirred vigorously for 15 minutes. The reaction was filtered through powdered cellulose to remove the catalyst, and the solvent was evaporated. The crude product was re-crystal]ized from hot EtOH/THF and dried under high vacuum to afford 189 mg (68%) of white, crystal]ine solid. ¹H NMR (400 MHz, DMSO-d₆) δ ppm 1.67 (m, 4 H) 3.21 (mr, 4 H) 8.17 (dd, J=8.71, 2.07 Hz, 1H) 8.20 (m, 2 H) 8.44 (d, J=2.07 Hz, 1H) 8.57 (s, 1H) 8.88 (d, J=8.91 Hz, 1H) 12.95 (s, 1H).

### Example 6.65: 5-Cyano-2-[({5-[(dipropylamino)suffonyl]-1,2-benzisoxazol-3-yl}carbonyl)amino]benzoic acid

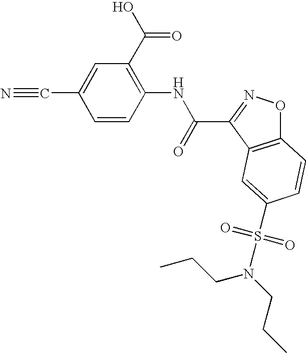

tert-Butyl

5-cyano-2-[({5-[(dipropylamino)sulfonyl]-1,2-benzisoxazol-3-yl}carbonyl)amino]benzoate (249 mg, 0.47 mmol) was dissolved in CH₂Cl₂ (10 mL) and treated with TFA (10 mL, Aldrich). After 3 hours, the solvent and excess TFA were evaporated, and the product was dried under vacuum at 100° C. overnight to afford 182 mg (quantitative) of off-white solid. ¹H NMR (400 MHz, DMSO-d₆) δ ppm 0.82 (t, J=7.36 Hz, 6 H) 1.50 (septet, J=7.41 Hz, 4 H) 3.09 (t, J=7.46 Hz, 4 H) 8.18 (m, 3 H) 8.44 (d, J=2.07 Hz, 1H) 8.57 (s, 1H) 8.89 (d, J=8.71 Hz, 1H) 12.96 (s, 1H).

### Example 6.66:

2-({[5-(Anilinosuffonyl)-1,2-benzisoxazol-3-yljcarbonyl}amino)-5-cyanobenzoic acid

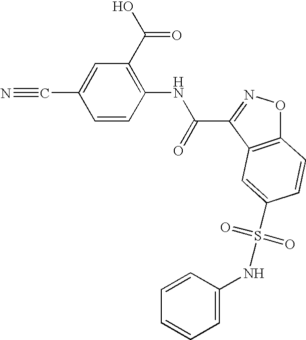

Preparation of tert-Butyl 2-({[5-(anilinosulfonyl)-1,2-benzisoxazol-3-yl]carbonyl}amino)-5-cyanobenzoate

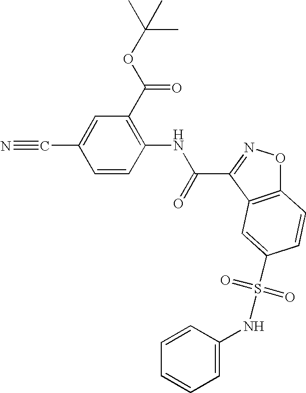

Prepared from aniline and tert9vutyl 2({[5-(chlorosulfonyl)-1,2-bensoxaol-3in 49% yield according to the general procedure except that CH₂Cl₂ was replaced by pyridine as the solvent. ¹H NMR (400 MHz, DMSO-d₆) δ ppm 1.59 (s, 9 H) 7.04 (t, J=7.26 Hz, 1H) 7.11 (d, J=7.46 Hz, 2 H) 7.23 (t, J=7.88 Hz, 2 H) 8.09 (dd, J=8.91, 1.66 Hz, 1H) 8.14 (d, J=9.12 Hz, 1H) 8.17 (dd, J=8.91, 1.87 Hz, 1H) 8.40 (d, J=2.07 Hz, 1H) 8.62 (d, J=1.24 Hz, 1H) 8.73 (d, J=8.71 Hz, 1H) 10.53 (s, 1H) 12.42 (s, 1H); ¹³C NMR (100 MHz, DMSO-d₆) δ ppm 27.64, 83.97, 106.50, 111.84, 117.92,119.22,119.59, 120.50, 121.49, 123.14, 124.56, 129.30, 135.31, 137.17, 137.21, 137.50, 142.32,152.36, 156.99, 164.84, 165.18; IR (diffuse reflectance) 2351 (w), 2309 (w), 2227, 2176 (w), 2113 (w), 1698, 1599, 1519 (s), 1498, 1330, 1295, 1276, 1259, 1161 (s), 1150 (s), cm⁻¹; HRMS (FAB) Calc. for C₂₆H₂₂N_{4O}₆S+H 519.1338, found 519.1323.

Prepared from the t-butyl ester in 40% yield according to general procedure. ¹H NMR (400 MHz, DMSO-d₆) δ ppm 7.04 (t, J=7.26 Hz, 1H) 7.12 (d, J=7.46 Hz, 2 H) 7.24 (t, J=7.88 Hz, 2 H) 8.08 (dd, J=8.91, 1.66 Hz, 1H) 8.12 (t, J=9.23 Hz, 1H) 8.17 (dd, J=8.92, 2.07 Hz, 1H) 8.44 (d, J=2.07 Hz, 1H) 8.63 (d, J=1.04 Hz, 1H) 8.86 (d, J=8.91 Hz, 1H) 10.53 (s, 1H) 12.93 (s, 1).

### Example 6.67: 5-Cyano-2-[((5-[(diethylamino)sulfonyl]-1,2-benzisoxazol-3-yl}carbonyl)amino]benzoic acid

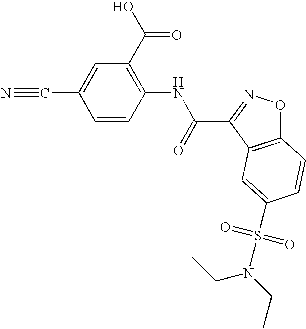

Preparation of Benzyl

5-cyano-2-[((5-[(diethylamino)sulfonyl]-1,2-benzisoxazol-3-yl}carbonyl)amino]benzoate

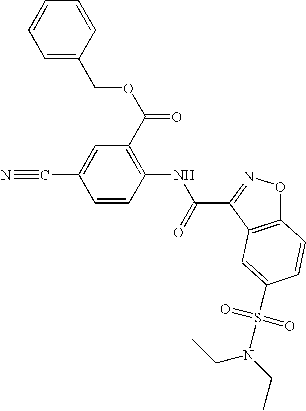

Prepared from diethylamine and benzyl 2-({[5-(chlorosulfonyl)-1,2-benzisoxazol-3-yl]carbonyl}amino)-5-cyanobenzoate in 44% yield according to the general procedure. ¹H NMR (400 MHz, DMSO-d₆) δ ppm 1.07 (t, J=7.05 Hz, 6 H) 3.23 (q, J=7.19 Hz, 4 H) 5.44 (s, 2 H) 7.35 (m, 3 H) 7.51 (d, J=1.24 Hz, 1H) 7.53 (d, J=1.66 Hz, 1H) 8.19 (d, J=1.04 Hz, 2 H) 8.22 (d, J=2.07 Hz, 1H) 8.49 (d, J=2.07 Hz, 1H) 8.56 (s, 1H) 8.75 (d, J=8.71 Hz, 1H) 12.42 (s, 1H); ¹³C NMR (100 MHz, DMSO-d₆) δ ppm 14.12, 41.94, 67.46, 106.72,112.02,117.78, 118.00, 119.63, 121.66, 122.79, 128.26, 128.34, 128.49, 129.47, 135.14, 137.54, 137.94, 142.37, 152.37, 157.07, 164.83, 165.69; IR (diffuse reflectance) 2394 (w), 2351 (w), 2328 (w), 2271 (w), 2232,1705 (s), 1590 (s), 1525 (s), 1346 (s), 1330, 1315, 1297 (s), 1272 (s), 1199 (s), 1153 (s), cm⁻¹; HRMS (ESI) Calc. for C₂₇H24N_{4O}₆S+H 533.1495, found 533.1492. Anal. Calc. for C₂₇ H24 N₄ 06 S: C, 60.89; H, 4.54; N, 10.52;S, 6.02. Found: C, 60.92; H, 4.67; N, 10.43.

The benzyl ester was converted to the acid according to the general procedure. ¹H NMR (400 MHz, DMSO-d₆) δ ppm 1.08 (t, J=7.15 Hz, 6 H) 3.23 (q, J=7.05 Hz, 4 H) 8.18 (m, 3 H) 8.44 (d, J=2.07 Hz, 1H) 8.58 (s, 1H) 8.88 (d, J=8.71 Hz, 1H) 12.95 (s, 1H).

### Example 6.68: 5-Cyano-2-({[5-(morpholin-4-ylsulfonyl)-1,2-benzisoxazol-3-yl]carbonyl}amino)benzoic acid

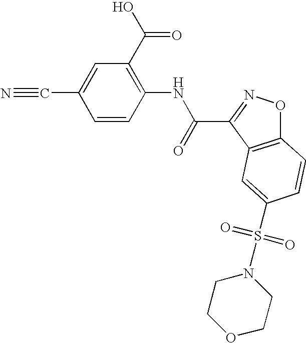

Preparation of tert-Butyl

5-cyano-2-({[5-(morpholin-4-ylsulfonyl)-1,2benzisoxazol-3-yl]carbonyl}amino)benzoate

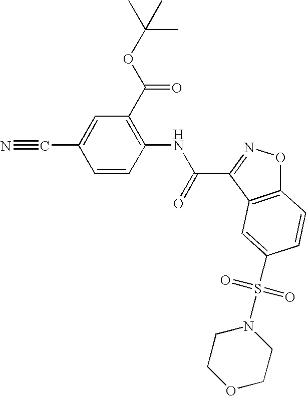

Prepared from morpholine and tert-butyl 2-({[5-(chlorosulfonyl)-1,2-benzisoxazol-3-yl]carbonyl}amino)-5-cyanobenzoate in 90% yield according to the general procedure. ¹H NMR (400 MHz, CDCl₃) δ ppm 1.67 (s, 9 H) 3.06 (m, 4 H) 3.76 (m, 4 H) 7.85 (dd, J=8.91, 2.07 Hz, 2 H) 8.04 (dd, J=8.92,1.87 Hz, 1H) 8.36 (d, J=1.87 Hz, 1H) 8.77 (d, J=1.04 Hz, 1H) 9.01 (d, J=8.91 Hz, 1H) 12.93 (s, 1H).

The t-butyl ester was converted to the acid according to the general procedure. ¹H NMR (400 MHz, DMSO-d₆) δ ppm 2.95 (m, 4 H) 3.65 (m, 4 H) 8.11 (dd, J=8.92, 1.87 Hz, 1H) 8.17 (dd, J=8.81, 1.97 Hz, 1H) 8.23 (d, J=8.71 Hz, 1H) 8.45 (d, J=2.07 Hz, 1) 8.53 (d, J=1.24 Hz, 1) 8.88 (d, J=8.71 Hz, 1H) 12.96 (s, 1H).

### Example 6.69: 5-Cyano-2-[({5-1(dimethylamino)sulfonyl]-1,2-benzisoxazol-3-yl}carbonyl)amino]benzoi acid

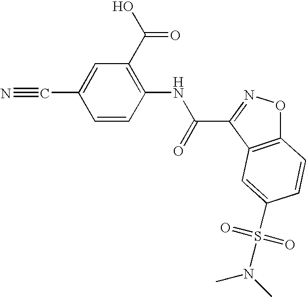

Preparation of tert-Butyl 5-cvano-2-1(f 5-[(dimethylamino)sulfonyl]-1,2benzisoxazol-3-yl]carbonyl)amino

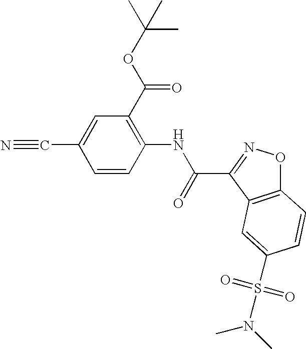

Prepared from dimethylamine and tert-butyl 2-({[5-(chlorosuifonyl)-1,2-benzisoxazol-3-yl]carbonyl}amino)-5-cyanobenzoate in 76% yield according to the general procedure except that CH₂Cl₂ was replaced by THF as the solvent. ¹H NMR (400 MHz, CDCl₃) δ ppm 1.68 (s, 9 H) 2.79 (s, 6 H) 7.85 (dd, J=8.81, 0.73 Hz, 1H) 7.86 (dd, J=8.81, 1.97 Hz, 1H) 8.09 (dd, J=8.81, 1.76 Hz, 1H) 8.37 (d, J=2.07 Hz, 1H) 8.80 (d, J=1.24 Hz, 1H) 9.03 (d, J=8.91 Hz, 1H) 12.92 (s, 1H).

The t-butyl ester was converted to the acid according to the general procedure. ¹H NMR (400 MHz, DMSO-d₆) δ ppm 2.68 (s, 6 H) 8.14 (dd, J=8.91, 1.66 Hz, 1H) 8.17 (dd, J=8.92, 2.07 Hz, 1H) 8.22 (d, J=8.71 Hz, 1H) 8.45 (d, J=2.07 Hz, 1H) 8.54 (d, J=1.24 Hz, 1H) 8.88 (d, J=8.71 Hz, 1H) 12.94 (s, 1H).

### Example 6.70:

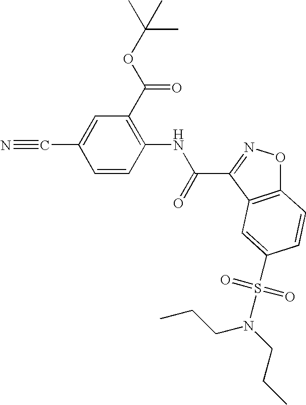

tert-Butyl

5-cyano-2-[({5-[(dipropylamino)sulfonyl]-1,2-benzisoxazol-3-yl}carbonyl)amino]benzoate Prepared from dipropylamine and tert-butyl 2-({[5-(chlorosulfonyl)-1,2-benzisoxazol-3-yl]carbonyl}amino)-5-cyanobenzoate according to the general procedure. ¹H NMR (400 MHz, DMSO-d₆) δ ppm 0.82 (t, J=7.36 Hz, 6 H) 1.50 (septet, J=7.26 Hz, 4 H) 1.61 (s, 9 H) 3.09 (t, J=7.46 Hz, 4 H) 8.19 (m, 3 H) 8.40 (d, J=2.07 Hz, 1 H) 8.56 (s, 1H) 8.73 (d, J=8.71 Hz, 1H) 12.44 (s, 1H); ¹³C NMR (100 MHz, DMSO-d₆) δ ppm 10.98, 21.61, 27.64, 49.65, 83.94, 106.56, 111.99, 117.91, 119.40, 119.65, 121.62,122.83, 129.55, 135.27, 137.30, 137.47, 142.24, 152.48, 157.07, 164.80, 165.18; IR (diffuse reflectance) 2403 (w), 2292 (w), 2232, 2182 (w), 1926 (w), 1705, 1585, 1520 (s), 1350, 1330, 1295, 1278, 1155 (s), 820, 610, cm⁻¹; Anal. Calc. for C₂₆ H₃₀ N₄ 06 S: C, 59.30; H, 5.74; N, 10.64;S, 6.09. Found: C, 59.21; H, 5.78; N, 10.52.

### Example 6.71: 2-[({5-[(5-Chloro-2,3-dihydro-1H-indol-1-yl)sulfonyl]-1,2benzisoxazol-3-yl}carbonyl)aminol-5-cyanobenzoic acid

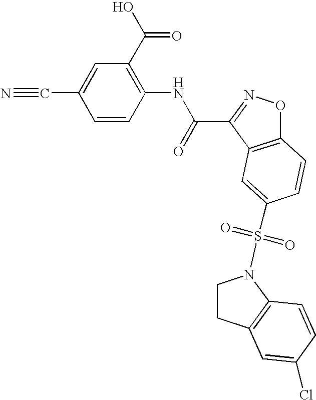

Preparation of

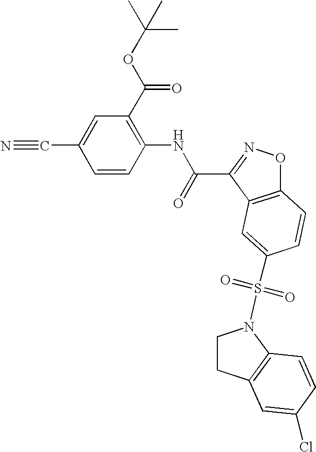

tert-Butyl 2-[(5-1(5-chloro-2,3-dihvdro-1H-indol-1-yl)sulfonyl]-1,2-benzisoxazol-3-yle carbonyl)aminol-5-cyanobenzoate

Prepared from 5-chloroindoline and tert-butyl 2-({[5-(chlorosulfonyl)-1,2benzisoxazol-3-yl]carbonyl}amino)-5-cyanobenzoate in 45% yield according to the general procedure. ¹H NMR (DMSO-d₆) δ ppm 1.60 (s, 9 H) 2.92 (t, J=8.40 Hz, 2 H) 3.99 (t, J=8.50 Hz, 2 H) 7.24 (d, J=1.87 Hz, 1H) 7.29 (dd, J=8.50, 2.28 Hz, 1H) 7.53 (d, J=8.50 Hz, 1H) 8.16 (br s, 2 H) 8.17 (dd, J=8.71, 2.07 Hz, 1H) 8.40 (d, J=2.07 Hz, 1H) 8.60 (br s, 1H) 8.73 (d, J=8.71 Hz, 1H) 12.42 (s, 1H).

The t-butyl ester was converted to the acid according to the general procedure. ¹H NMR (DMSO-d₆) δ ppm 2.92 (t, J=8.40 Hz, 2 H) 3.99 (t, J=8.40 Hz, 2 H) 7.24 (s, 1 H) 7.30 (dd, J=8.60, 2.18 Hz, 1H) 7.54 (d, J=8.71 Hz, 1H) 8.15 (br d, 2 H) 8.17 (dd, 15 J=8.91, 2.07 Hz, 1H) 8.44 (d, J=2.07 Hz, 1H) 8.61 (s, 1H) 8.87 (d, J=8.71 Hz, 1H) 12.95 (s, 1H)

### Example 6.72: N,N-Diethylammonium

5-cyano-2-1({5-[(methylamino)sulfonyl]1,2-benzisoxazol-3-yl}carbonyl)amino]benzoate

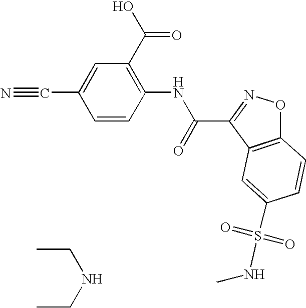

Preparation of

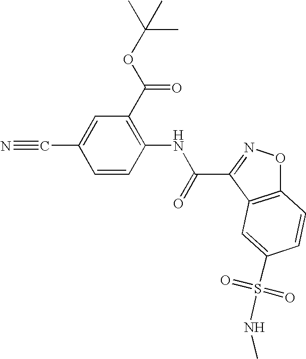

tert-Butyl

5-cyano-2-[(f 5-1(methylamino)sulfonyl]-1.2-benzisoxazol-3-yl]carbonyl)amino]benzoate

Prepared from methylamine and tert-butyl 2-({[5(chlorosulfonyl)-1,2-benzisoxazol-3-yl]carbonyl}amino)-5-cyanobenzoate in 99% yield according to the general procedure except that CH₂Cl₂ was replaced by THF as the solvent. Product was used without purification due to very poor solubility in organic solvents. ¹H NMR (DMSO-d₆) δ ppm 1.60 (s, 9 H) 2.44 (d, J=4.98 Hz, 3 H) 7.75 (q, J=4.98 Hz, 1H) 8.16 (m, 3 H) 8.41 (d, J=2.07 Hz, 1H) 8.61 (d, J=1.04 Hz, 1H) 8.76 (d. J=8.92 Hz. 1H) 12.46 (s, 1H).

tert-Butyl

5-cyano-2-[({5-[(methylamino)sulfonyl]-1,2-benzisoxazol-3-yl}carbonyl)amino]benzoate (225 mg, 0.49 mmol) was dissolved in CH₂Cl₂ (10 mL) and treated with TFA (10 mL, Aldrich). A pinkish precipitate formed. After stirring overnight, the solvent and excess TFA were evaporated, and the residue was triturated in CH₃OH. The product was impure but would not dissolve for purification, so it was heated at 80° C. in a sealed tube with a mixture of diethylamine and THF to form the salt. The solvent was evaporated, and the crude salt was re-crystal]ized from a hot mixture of EtOH and 1,2-DCE. The crystals were washed with CH₃OH followed by heptane and then dried several days under vacuum at 100° C. to afford 64 mg (28%) of tan solid. ¹H NMR (DMSO-d₆) δ ppm 1.18 (t, J=7.26 Hz, 6 H) 2.45 (d, J=4.98 Hz, 3 H) 2.94 (br s, 4 H) 7.73 (q, J=4.77 Hz, 1H) 7.92 (dd, J=8.50, 2.07 Hz, 1H) 8.12 (dd, J=8.91, 1.87 Hz, 1H) 8.17 (d, J=8.91 Hz, 1) 8.37 (d, J=2.07 Hz, 1H) 8.43 (br s, 2 H) 8.65 (d, J=1.66 Hz, 1H) 8.81 (d, J=8.71 Hz, 1H).

### Example 6.73: 2-{[(5-{[Bis(2-hydroxyethyl)aminolsulfonyl}-1,2-benzisoxazol-3-yl)carbonyl]amino}-5-cyanobenzoic acid

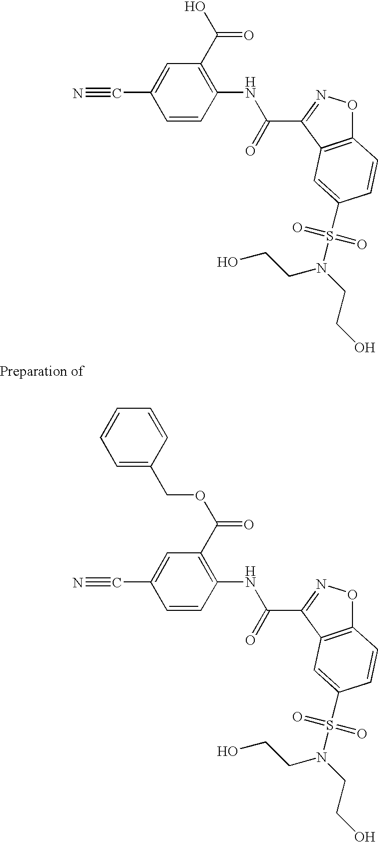

Benzyl 2-{[(5-{[bis(2-hydroxyethyl)aminol sulfonyl}-1,2-benzisoxazol-3-yl)carbonyl]amino}-5-cyanobenzoate

Prepared from diethanolamine and benzyl 2({[5-(chlorosulfonyl)-1,2-benzisoxazol-3-yl]carbonyl}amino)-5-cyanobenzoate in 49% yield according to the general procedure except that triethylamine (1.1 eq) was added. ¹H NMR (400 MHz, DMSO-d₆) δ ppm 3.24 (t, J=6.32 Hz, 4 H) 3.53 (br s, 4 H) 4.84 (s, 2 H) 5.45 (s, 2 H) 7.36 (m, 3 H) 7.52 (d, J=6.63 Hz, 2 H) 8.20 (m, 3 H) 8.49 (d, J=2.07 Hz, 1H) 8.58 (s, 1H) 8.76 (d, J=8.92 Hz, 1H) 12.43 (s, 1H).

The benzyl ester was converted to the acid according to the general procedure. ¹H NMR (400 MHz, DMSO-d₆) δ ppm 3.24 (t, J=6.32 Hz, 4 H) 3.54 (t, J=6.22 Hz, 4 H) 4.86 (s, 2 H) 8.18 (m, 3 H) 8.45 (d, J=2.07 Hz, 1H) 8.61 (s, 1H) 8.89 (d, J=8.91 Hz, 1H) 13.16 (s, 1H); 1³C NMR (100 MHz, DMSO-d₆) δ ppm 50.82, 59.63, 106.11, 111.85, 118.00, 118.32,119.61, 120.69, 123.16, 129.65, 135.51, 137.00, 137.54, 143.14, 152.61, 157.07, 164.88, 168.01; IR(diffiusereflectance)2250,2250,2227 (w),2198(w), 1965(w), 1915(w), 1704, 1591, 1520(s), 1339, 1297, 1179, 1150(s), 987, 731, cm⁻¹; Anal. Calc. for C₂₀ H₁₈ N₄ 08 S: C, 50.63; H, 3.82; N, 11.81;S, 6.76. Found: C, 50.50; H, 3.76; N, 11.75.

### Example 6.74: 5-Cyano-2-[({5-nitro-6-[4-nitro-2-(trifluoromethyl)phenyl]-1,2benzisoxazol-3-yl}carbonyl)amino]benzoic acid

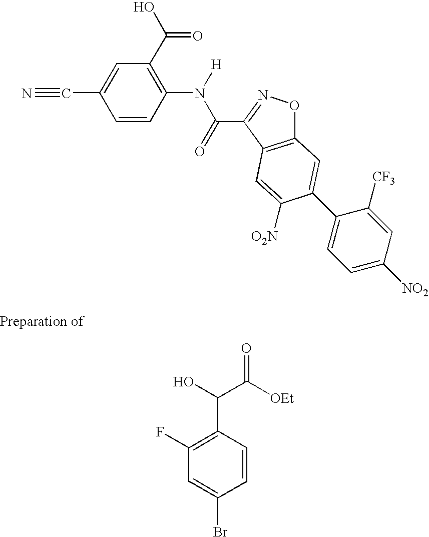

Ethyl (4-bromo-2-fluorophenyl)(hydroxy)acetate Trimethylsilyl cyanide (15.1 mL, 113 mmol, Aldrich) was added to a solution of DABCO (0.40 g, 3.6 mmol, Fluka) and 4-bromo-2-fluorobenzaldehyde (23.1 g, 114 numol, Fluka) in CH₂Cl₂ (350 mL) with cooling in an ice bath. The reaction mixture was stirred at 0° C. for 30 minutes and then at room temperature for 2 hours. The mixture was washed with water (2×200 mL) and brine (200 mL), dried over Na₂SO₄, and evaporated leaving the silylated cyanohydrin as 34.4 g of cloudy oil. This material was treated with water (25 mL) and concentrated HCl (75 mL) and then heated at reflux for 1.7 hours. The mixture was diluted with water (300 mL) and made basic with 6 M NaOH. This solution was washed with CH₂Cl₂ (2×250 mL) and then acidified with concentrated HCl. The carboxylic acid was extracted into CH₂Cl₂ (2×250 mL). The CH₂Cl₂ was dried over Na₂SO₄ and evaporated yielding 21.3 g of white solid. This carboxylic acid (20.1 g) was dissolved in DMF (300 mL). Cesium carbonate (27.7 g, 85.0 mmol, Aldrich) and iodoethane (6.25 mL, 78.1 mmol, Aldrich) were added, and the reaction was stirred at room temperature for 3 hours and then at 60° C. for 2.5 hours. The mixture was diluted with MTBE (1 L) and washed with water (500 mL), saturated NaHCO₃ (500 mL), water (2×500 mL), and brine (500 mL). The organics were dried over Na₂SO₄, filtered through a 1″ plug of silica gel, and evaporated yielding the ethyl ester as 19.8 g of off-white solid. ¹H NMR (400 MHz, DMSO-d₆, ¹⁹F decoupled) δ ppm 1.13 (t, J=7.15 Hz, 3 H) 4.10 (m, 2 H) 5.29 (d, J=5.60 Hz, 1H) 6.32 (d, J=5.60 Hz, 1H) 7.44 (m, 2 H) 7.56 (d, J=1.45 Hz, 1H).

Preparation of

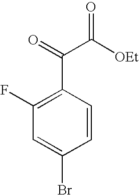

Ethyl (4-bromo-2-fluorophenyl)(oxo)acetate Acetic anhydride (10 mL, 110 mmol) was added dropwise to a solution of ethyl (4-bromo-2-fluorophenyl)(hydroxy)acetate (18.5 g, 66.8 mmol) in DMSO (50 μL) at 100° C. Heat was removed after 2.3 hours. The mixture was diluted with MTBE (250 mL) and washed with brine (3×250 mL). The organics were filtered through a 1″ plug of silica gel and evaporated leaving a golden oil. This material was purified on a Biotage Flash 75M silica gel cartridge using 50% CH₂Cl₂ in heptane as eluent. Yield was 1 7.0 g of golden oil. ¹H NMR (400 MHz, DMSO-d₆, ¹⁹F decoupled) δ ppm 1.31 (t, J=7.05 Hz, 3 H) 4.39. (q, J=7.19 Hz, 2 H) 7.69 (dd, J=8.29, 1.66 Hz, 1H) 7.85 (d, J=8.29 Hz, 1H) 7.89 (d, J=1.66 Hz, 1H).

Preparation of

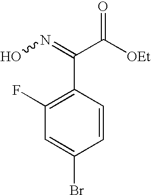

Ethyl (4-bromo-2-fluorophenyl)(hydroxyinino)ethanoate Hydroxylamine hydrochloride (4.77 g, 68.6 mmol, Mal]inckrodt) and sodium acetate (6.11 g, 74.5 mmol) were added to a solution of ethyl (4-bromo-2-fluorophenyl)(oxo)acetate (15.8 g, 57.5 mmol) in ethanol (45 mL), and the mixture was stirred at room temperature overnight. After removal of the ethanol by rotary evaporation, the residue was dissolved in ethyl acetate (250 mL). This solution was washed with water (250 mL), brine (250 mL), and saturated NaHCO₃ (2×250 mL). The organics were dried over Na₂SO₄ and evaporated yielding 15.9 g of white solid (mixture of 2 oxime isomers). ¹H NMR (400 MHz, DMSO-d₆, ¹⁹F decoupled, mrajor isomer) δ ppm 1.24 (t, J=7.26 Hz, 3 H) 4.23 (q, J=7.12 Hz, 2 H) 7.36 (d, J=8.29 Hz, 1H) 7.50 (dd, J=8.19, 1.76 Hz, 1H) 7.66 (d, J=1.66 Hz, 1H) 12.93 (s, 1H).

Preparation of

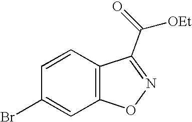

Ethyl 6-bromo-1,2-benzisoxazole-3-carboxylate Potassium carbonate (10.5 g, 76.0 mmol) was added to a solution of ethyl (4-bromo-2fluorophenyl)(hydroxyimino)ethanoate (15.4 g, 53.0 mmol, mixture of isomers) in DMSO (50 mL) at 75 C. Heat was removed after 5 hours, and the mixture was diluted with water (300 mL). Product was extracted into CH₂Cl₂ (2×250 mL). The combined CH₂Cl₂ was washed with water (250 mL) and brine (250 mL). The organics were filtered through a 1″ plug of silica gel and evaporated leaving a white solid. This material was purified on a Biotage Flash 75 M silica gel cartridge using 50% CH₂Cl₂ in heptane as eluent. Yield was 11.2 g of white solid. ¹H NMR (400 MHz, DMSO-d₆) δ ppm 1.40 (t, J=7.15 Hz, 3 H) 4.49 (q, J=7.05 Hz, 2 H) 7.73 (dd, J=8.50, 1.45 Hz, 1H) 8.00 (d, J=8.50 Hz, 1H) 8.33 (d, J=1.66 Hz, 1H).

Preparation of

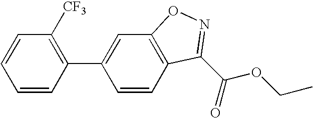

Ethyl 6[2-(trifluoromethyl)phenyl]-1,2-benzisoxazole-3-carboxylate Toluene (40 mL) was added to a flask containing ethyl 6-bromo-1,2-benzisoxazole-3-carboxylate (3.15 g, 11.7 mmol), tetrakis(triphenylphosphine)pal]adium(O) (835 mg, 0.723 mmol, Strem), cesium carbonate (4.12 g, 12.6 mmol, Aldrich), and 2(trifluoromethyl)phenylboronic acid (2.38 g, 12.5 mmol, Aldrich) under argon. This mixture was heated in a 100° C. oil bath for 35 hours. The mixture was diluted with EtOAc (250 mL) and washed with 4:1 water:brine (250 mL) followed by brine (250 mL). The organics were filtered through a 1″ plug of silica gel and evaporated. Product was split in two, adsorbed onto silica gel, and purified on Biotage Flash 40 M+silica cartridges using 20% EtOAc in heptane. Yield was 2.08 g of orange solid. ¹H NMR (400 MHz, DMSO-d₆) δ ppm 1.42 (t, J=7.15 Hz, 3 H) 4.51 (q, J=7.05 Hz, 2 H) 7.52 (d, J=7.46 Hz, 1H) 7.51 (d, J=8.09 Hz, 1H) 7.71 (t, J=7.57 Hz, 1H) 7.79 (t, J=7.36 Hz, 1H) 7.91 (d, J=7.67 Hz, 1H) 7.93 (s, 1H) 8.14 (d, J=8.09 Hz, 1H).

Preparation of

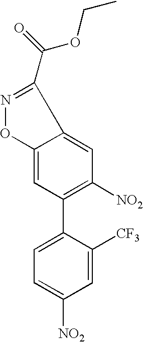

Ethyl 5-nitro-6-[4-nitro-2-(trifluoromethyl)phenyl]-1,2-benzisoxazole-3-carboxylate Nitric acid (5.0 μL, Mal]inckrodt, 69%) was added dropwise to a suspension of ethyl 6-[2-(trifluoromethyl)phenyl]-1,2-benzisoxazole-3-carboxylate (2.80 g, 8.61 mmol) in sulfuric acid (20 μL, Mal]inckrodt, 96%) with stirring in an ice bath. The mixture was allowed to warm slowly to room temperature overnight. The mixture was poured over ice, and the product was extracted into CH₂Cl₂ (2×100 mL). The product precipitated from the CH₂Cl₂, so EtOAc (200 mL) was added. The organics were washed with saturated NaHCO₃, filtered through a plug of silica gel, and evaporated. Product was purified on a Biotage Flash 40 M+cartridge using 25% EtOAc in heptane. Yield was 2.37 g of white solid that was used without further purification despite only being approximately 80% pure. ¹H NMR (400 MHz, DMSO-d₆) δ ppm 1.44 (t, J=7.05 Hz, 3 H) 4.56 (q, J=7.12 Hz, 2 H) 7.92 (d, J=8.29 Hz, 1H) 8.31 (s, 1H) 8.63 (d, J=2.28 Hz, 1H) 8.66 (dd, J=8.29, 2.28, 1 H) 8.92 (s, 1H).

Preparation of

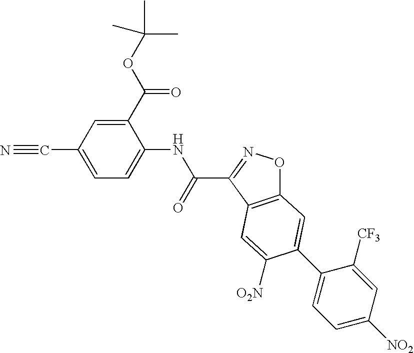

tert-Butyl 5-cyano-2-[({5-nitro-6-[4-nitro-2-(trifluoromethyl)phenyl]-1,2-benzisoxazol-3-yl}carbonyl)amino]benzoate Sulfuric acid (25 mL, 80% in water) was added to a flask containing ethyl 5-nitro-6-[4-nitro-2-(trifluoromethyl)phenyl]-1,2-benzisoxazole-3-carboxylate (2.33 g, 5.48 mmol), and the mixture was stirred in a 75° C. oil bath for 2 hours. (Additional heating or higher temperatures can cause extensive hydrolysis of the trifluoromethyl group). The mixture was poured over ice and extracted with CH₂Cl₂ (2×100 mL). The CH₂Cl₂ was dried over Na₂SO₄ and evaporated. The residue was suspended in CH₂Cl₂ (50 μL) and treated with DMP (50 μL) followed by oxalyl chloride (6 mL). An additional 1 mL portion of oxalyl chloride was added after 14.5 hours, but no bubbles were observed, so the solvent and excess oxalyl chloride were removed by rotary evaporation. Heptane (50 mL) was added and then removed by rotary evaporation to remove the residual oxalyl chloride. The residue was dissolved in CH₂Cl₂ (30 mL) and treated with t-butyl 2-amino-5-cyanobenzoate (1.18 g, 5.41 mmol) in pyridine (15 mL). After 2 hours, the reaction was diluted with CH₂Cl₂ (100 mL) and THF (20 mL). The mixture was washed with 1 M HCl (100 mL). There solids were filtered away, washed with heptane, and dried under vacuum yielding 1.15 g of white solid that was used without further purification. ¹H NMR (400 MHz, DMSO-d₆) δ ppm 1.62 (s, 9 H) 7.94 (d, J=8.50 Hz, 1H) 8.21 (dd, J=8.71, 2.07 Hz, 1H) 8.35 (s, 1H) 8.44 (d, J=1.87 Hz, 1H) 8.64 (d, J=2.28 Hz, 1H) 8.66 (dd, J=8.48, 2.28 Hz, 1H) 8.77 (d, J=8.71 Hz, 1H) 9.04 (s, 1H) 12.49 (s, 1H).

5-Cyano-2-[({5-nitro-6-[4-nitro-2-(trifluoromethyl)phenyl]-1,2-benzisoxazol-3-yl}carbonyl)amino]benzoic acid

Trifluoroacetic acid (15 mL) was added to a slurry of the corresponding t-butyl ester (366 mg, 0.613 mmol) in CH₂Cl₂ (20 mL). The resulting solution was stirred for 5.5 hours. The solvents were removed by rotary evaporation. Methanol was added and then removed by rotary evaporation. The residue was recrystallized from hot ethanol (10 mL) with a few drops of THF added. The crystals were washed with methanol followed by heptane and then dried at 100° C. yielding 177 mg of off-white solid. ¹H NMR (400 MHz, DMSO-d₆) δ ppm 7.93 (d, J=8.29 Hz, 1H) 8.20 (dd, J=8.71, 2.07 Hz, 1H) 8.34 (s, 1H) 8.47 (d, J=2.07 Hz, 1H) 8.64 (d, J=2.07 Hz, 1H) 8.66 (dd, J=8.29, 2.07 Hz, 1H) 8.89 (d, J=8.71 Hz, 1H) 9.05 (s, 1H) 13.04 (s, 1H).

### Example 6.75: 2-[({5-(Acetylamino)-6-[4-(acetylamino)-2(trifluoromethyl)phenyl]-1,2-benzisoxazol-3-yl}carbonyl)amino]-5-cyanobenzoic acid

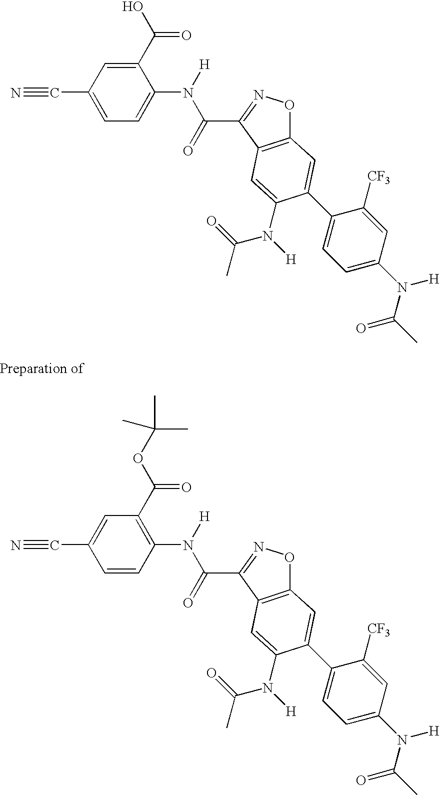

tert-Butyl 2-[({5-(acetylamino)-6-[4-(acetylamino)-2-(trifluoromethyl)phenyl-1,2-benzisoxazol-3-yl}carbonyl)amino]-5-cyanobenzoate Tin (II) chloride dihydrate (5.25 g, 23.3 mmol, Aldrich) was added to a suspension of tert-butyl

5-cyano-2-[({5nitro-6-[4-nitro-2-(trifluoromethyl)phenyl]-1,2-benzisoxazol-3-yl}carbonyl)amimo]benzoate (1.08 g, 1.81 mmol) in amixture of DMF (40 μL) and THF (40 mL). The mixture was stirred for 7 days and then diluted with MTBE (250 mL). It was washed with 1 M NaOH (3×100 mL) and brine (100 mL). The organics were filtered through a plug of silica and evaporated yielding 826 mg of orange solid. This material was dissolved in CH₂Cl₂ (25 mL) and treated with triethylamine (560 μL, 4.03 mmol, Aldrich) followed by acetyl chloride (250 μL, 3.52 mmol, Aldrich). The mixture was stirred for 15 hours and then treated with an additional portion of acetyl chloride (100 μL). After an additional 30 minutes, the mixture was filtered through a plug of silica. Product was adsorbed onto silica and purified on a Biotage Flash 40 M+using a gradient from 60% to 80% EtOAc in CH₂Cl₂. Yield was 455 mg of white solid.

¹H NMR (400 MHz, DMSO-d₆) δ ppm 1.62 (s, 9 H) 1.87 (s, 3 H) 2.12 (s, 3 H) 7.30 (d, J=8.50 Hz, 1H) 7.80 (s, 1H) 7.84 (dd, J=8.50, 1.66 Hz, 1H) 8.17 (dd, J=8.71, 2.07 Hz, 1H) 8.21 (d, J=1.87 Hz, 1H) 8.41 (d, J=2.07 Hz, 1H) 8.45 (s, 1H) 8.80 (d, J=8.71 Hz, 1H) 9.01 (s, 1H) 10.41 (s, 1H) 12.42 (s, 1H).

Trifluoroacetic acid (10 μL) was added to a slurry of the corresponding t-butyl ester in CH₂Cl₂ (15 mL), and the resulting solution was stirred overnight. Solvents were removed by rotary evaporation, and the product was triturated with methanol. Product was washed with heptane and dried at 100° C. under vacuum yielding 298 mg of tan solid. ¹H NMR (400 MHz, DMSO-d₆) δ ppm 1.88 (s, 3 H) 2.12 (s, 3 H) 7.30 (d, J=8.29 Hz, 1H) 7.79 (s, 1H) 7.84 (dd, J=8.60, 1.14 Hz, 1H) 8.18 (dd, J=8.71, 1.87 Hz, 1H) 8.21 (d, J=1.66 Hz, 1H) 8.46 (m, 2 H) 8.91 (d, J=8.71 Hz, 1H) 9.02 (s, 1H) 10.41 (s, 1H) 12.92 (s, 1H).

### Example 7.76: 2-[({5-(Acetylamino)-6-[4-(acetylamino)-2(trifluoromethyl)phenyl]-1,2-benzisoxazol-3-yl}carbonyl)amino]-5-bromobenzoic acid

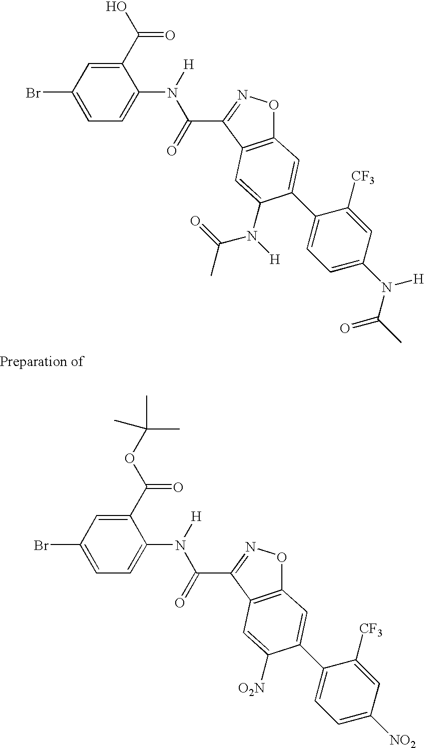

tert-Butyl 5-bromo-2-[({5-nitro-6-[4-nitro-2-(trifluoromethyl)phenyl]-1,2-benzisoxazol-3-yl Sulfuric acid (25 mL, 80% in water) was added to ethyl 6-[2-(trifuoromethyl)phenyl]-1,2-benzisoxazole-3-carboxylate (2.04 g, 6.27 mmol), and the mixture was stirred in a 75° C. oil bath for 2.25 hours while monitoring the reaction to avoid hydrolysis of the trifluoromethyl group. The mixture was poured over ice, and the product was extracted into EtOAc (2×75 mL). The organics were dried over Na₂SO₄ and evaporated leaving a brown solid. This material was suspended in H₂SO₄ (20 mL, 96%) and treated with nitric acid (5 mL, 69%) with stirring in an ice bath. The mixture was allowed to warm to room temperature overnight and was then poured over ice. Product was extracted into EtOAc (2×100 mL). The organics were dried over Na₂SO₄ and evaporated. Toluene (2×100 mL) was added and then evaporated to help rid the material of acetic acid. The resulting orange solid (2.52 g) was suspended in CH₂Cl₂ (50 mL) and treated with DMF (20 μL) followed by oxalyl chloride (6 mL). An additional portion of oxalyl chloride (1 mL) was added after 1 hour, and the mixture was stirred for an additional 1 hour. Solvent and excess oxalyl chloride were removed by rotary evaporation. The residue was dissolved in CH₂Cl₂ (30 mL) and treated with t-butyl 2amimo-5-bromobenzoate (1.44 g, 5.29 mmol) in pyridine (10 mL). The mixture was stirred for 70 minutes and then added to a separatory fumnel with CH₂Cl₂ (100 mL). The organics were washed with 1 M HCl (2×100 mL) and brine (100 mL). Product was adsorbed onto silica and purified on a Biotage Flash 40 M+silica cartridge using a gradient from 60% to 80% CH₂Cl₂ in heptane yielding 1.61 g of white solid. This material was further purified by recrystal]ization from hot toluene (approximately 75 mL). Yield was 830 mg of white solid that could not be completely freed from residual toluene, even after extended heating at 100° C. under vacuum. ¹H NMR (400 MHz, DMSO-d₆) δ ppm 1.60 (s, 9 H) 7.95 (m, 2 H) 8.09 (d, J=2.49 Hz, 1H) 8.33 (s, 1H) 8.52 (d, J=8.91 Hz, 1H) 8.63 (d, J=2.07 Hz, 1H) 8.66 (dd, J=8.28, 2.28 Hz 1H) 9.03 (s, 1H) 12.20 (s, 1H).

Preparation of

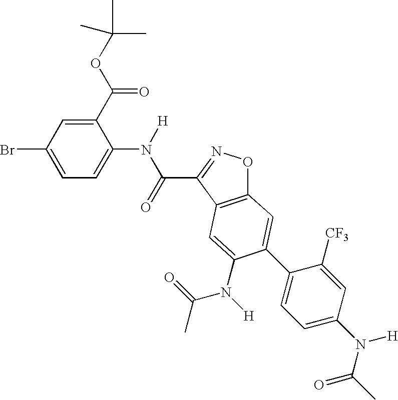

tert-Butyl 2-1({5-(acetylamino)-6-[4-(acetylamino)-2-(trifluoromethyl)phenyl]-1,2-benzisoxazol-3-yl}carbonyl)amino]-5bromobenzoate Tin (II) chloride dihydrate (4.05 g, 17.9 mmol, Aldrich) was added to a mixture of tert-butyl 5-bromo-2-[({5nitro-6-[4-nitro-2-(trifluoromethyl)phenyl]1,2-benzisoxazol-3-yl}carbonyl)amino]benzoate (715 mg, 1.10 mmol) in a mixture of THF (40 mL) and DMF (40 mL). The mixture was stirred for 6 days and then diluted with MTBE (250 mL). The organics were washed with 1 M NaOH (3×100 mL) and brine (100 mL). 10 It was Luthe ilteIuU tLhoiughii a plug of silica arid evaporated. Tne resulting orange solid was dissolved in CH₂Cl₂ (25 mL) and treated with triethylamine (560 μL) and acetyl chloride (250 μL). After 5 hours, the mixture was filtered through a plug of silica gel. Product was then adsorbed onto silica gel and purified on a Biotage Flash 40 M+using a gradient from 50% EtOAc to 75% EtOAc in CH₂Cl₂. Yield was 161 mg of light yellow solid.

¹H NMR (400 MHz, DMSO-d₆) δ ppm 1.59 (s, 9 H) 1.87 (s, 3 H) 2.12 (s, 3 H) 7.29 (d, J=8.50 Hz, 1H) 7.78 (s, 1H) 7.84 (dd, J=8.40, 1.76 Hz, 1H) 7.92 (dd, J=8.91, 2.49 Hz, 1H) 8.08 (d, J=2.49 Hz, 1H) 8.21 (d, J=1.87 Hz, 1H) 8.43 (s, 1H) 8.56 (d, J=8.91 Hz, 1H) 9.01 (s, 1H) 10.40 (s, 1H) 12.11 (s, 1H).

A slurry of the t-butyl ester (137 mg, 0.203 mmol) in CH₂Cl₂ (12 mL) was treated with TFA (8 mL), and the resulting solution was stirred overnight. Solvents were removed by rotary evaporation, and the product was triturated with methanol. The acid product was washed with heptane and dried at 100° C. under vacuum yielding 49 mg of tan solid. ¹H NMR (400 MHz, DMSO-d₆) δ ppm 1.87 (s, 3 H) 2.12 (s, 3 H) 7.29 (d, J=8.50 Hz, 1H) 7.78 (s, 1H) 7.84 (dd, J=8.19, 1.35 Hz, 1H) 7.93 (dd, J=9.02, 2.38 Hz, 1H) 8.17 (d, J=2.49 Hz, 1H) 8.21 (d, J=1.45 Hz, 1H) 8.45 (s, 1H) 8.71 (d, J=9.12 Hz, 1H) 9.01 (s, 1H) 10.40 (s, 1H) 12.63 (s, 1H). cl Example 6.77: 5-Cyano-2-[({6-[2-(trifluoromethyl)phenyl]-1,2-benzisoxazol-3-yl}carbonyl)amino]benzoic acid

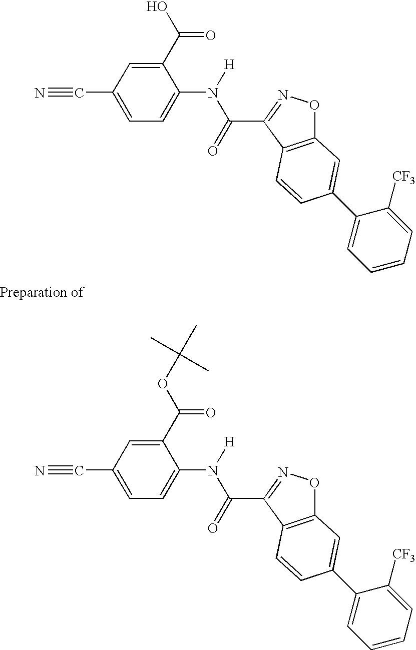

tert-Butyl

5-cyano-2-[({6-[2-(trifluoromethyl)phenyl]-1,2-benzisoxazol-3-yl}carbonyl)amino]benzoate Sulfuric acid (40 mL, 75% in water) was added to a flask containing ethyl 6-[2-(trifluoromethyl)phenyl]-1,2-benzisoxazole-3-carboxylate (1. 1 2 g, 3.35 mmol), and the mixture was stirred in a 60-80° C. oil bath for 80-110 minutes. The mixture was diluted to 120 μL with water, and the resulting precipitate was collected and air dried yielding 862 mg of yellow solid. This material was suspended in CH₂Cl₂ (100 mL) and treated with DMF (30 μL) followed by oxalyl chloride (2 μL). Solvent and excess oxalyl chloride were removed by rotary evaporation after 1 hour. The residue was dissolved in CH₂Cl₂ (75 mL) and treated with t-butyl 2-amino-5-cyanobenzoate (552 mg, 2.53 mmol) in pyridine (20 mL). The mixture was stirred for 16.5 hours and then diluted with CH₂Cl₂ (100 mL). It was washed with 1 M HCl (2×150 mL) followed by brine (150 mL). Product was adsorbed onto silica gel and purified on a Biotage Flash 40 M+silica cartridge using a gradient from 50% to 75% CH₂Cl₂ in heptane. Yield was 446 mg of white solid. ¹H NMR (400 MHz, DMSO-d₆) δ ppm 1.62 (s, 9 H) 7.53 (d, J=8.29 Hz, 2 H) 7.71 (t, J=7.57 Hz, 1H) 7.80 (t, J=7.36 Hz, 1H) 7.92 (d, J=7.67 Hz, 1H) 7.97 (s, 1H) 8.18 (dd, J=8.71, 2.07 Hz, 1H) 8.26 (d, J=8.09 Hz, 1H) 8.41 (d, J=2.07 Hz, 1H) 8.80 (d, J=8.71 Hz, 1H) 12.43 (s, 1H).

A solution of the corresponding t-butyl ester (371 mg, 0.731 mmol) in CH₂Cl₂(15 mL) was treated with TFA (10 mL) and stirred for 3 hours. Solvents were removed by rotary evaporation. To remove residual TFA, the residue was suspended in CH₂Cl₂, and the solvent was removed by rotary evaporation. This was repeated with methanol and with heptane. The acid product was then triturated with methanol, washed with heptane, and dried at 100° C. under vacuum yielding 310 mg of white solid. ¹H NMR (400 MHz, DMSO-d₆) δ ppm 7.53 (d, J=8.09 Hz, 2 H) 7.71 (t, J=7.57 Hz, 1 H) 7.80 (t, J=7.36 Hz, 1H) 7.92 (d, J=7.46 Hz, 1H) 7.96 (s, 1H) 8.18 (dd, J=8.71, 2.07 Hz, 1H) 8.27 (d, J=8.09 Hz, 1H) 8.46 (d, J=2.07 Hz, 1H) 8.92 (d, J=8.91 Hz, 1H) 12.92 (s, 1H).

### Example 6.78: Other examples of R₄ as optionally substituted benzisoxazole

| Compound No., Structure | Compound Name |
|---|---|
| 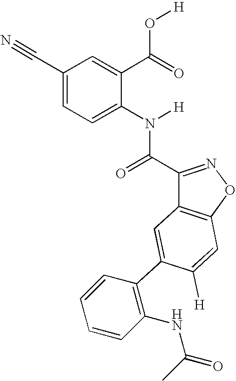 | 2-[({5-[2-(acetylamino)phenyl]-1,2- benzisoxazol-3-yl}carbonyl)amino]-5-cyanobenzoic acid |
| 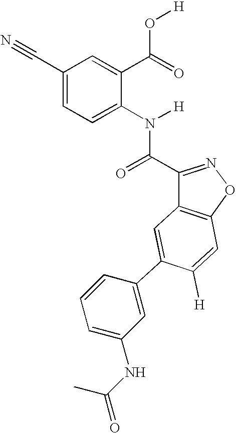 | 2-[({5-[3-(acetylamino)phenyl]-1,2- benzisoxazol-3-yl}carbonyl)amino]-5-cyanobenzoic acid |
| 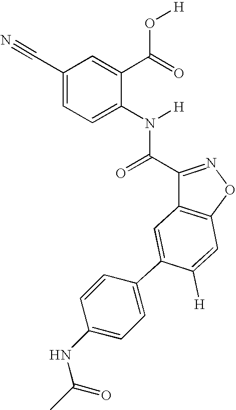 | 2-[({5-[4-(acetylamino)phenyl]-1,2- benzisoxazol-3-yl}carbonyl)amino]-5-cyanobenzoic acid |
| 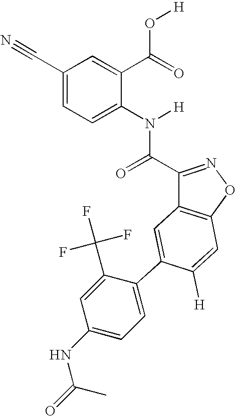 | 2-[({5-[4-(acetylamino-2-(trifluoromethyl)phenyl]- 1,2-benzisoxazol-3-yl}carbonyl)amino]-5- cyanobenzoic acid |
| 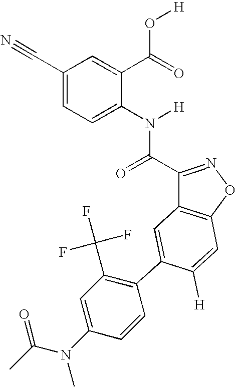 | 2-[({5-[4-[acetyl(methyl)amino]-2- (trifluoromethyl)phenyl]-1,2-benzisoxazol-3- yl}carbonyl)amino]-5-cyanobenzoic acid |
| 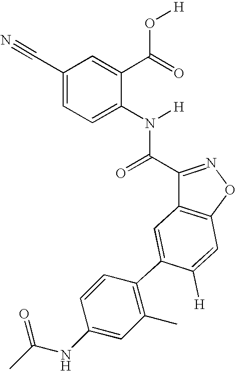 | 2-[({5-[4-(acetylamino)-2-methylphenyl]-1,2-benzisoxazol- 3-yl}carbonyl)amino]-5-cyanobenzoic acid |
| 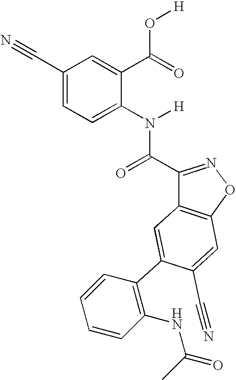 | 2-[({5-[2-(acetylamino)phenyl]-6-cyano-1,2-benzisoxazol- 3-yl}carbonyl)amino]-5-cyanobenzoic acid |
| 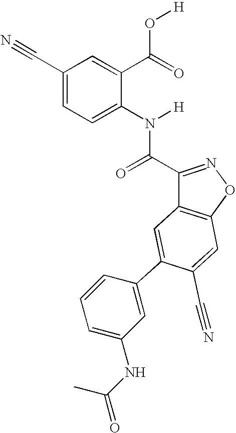 | 2-[({5-[3-(acetylamino)phenyl]-6-cyano-1,2-benzisoxazol- 3-yl}carbonyl)amino]-5-cyanobenzoic acid |
| 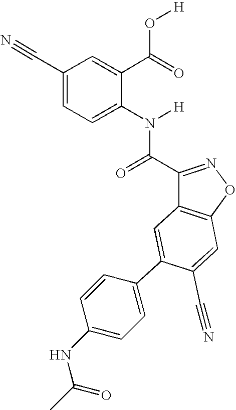 | 2-[({5-[4-(acetylamino)phenyl]-6-cyano-1,2-benzisoxazol- 3-yl}carbonyl)amino]-5-cyanobenzoic acid |
| 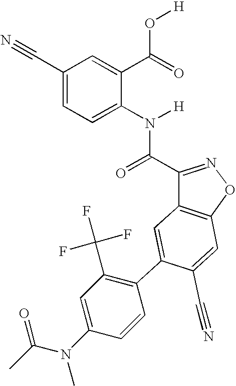 | 2-[({5-[4-[acetyl(methyl)amino]-2-(trifluoromethyl)phenyl]- 6-cyano-1,2-benzisoxazol-3-yl}carbonyl)amino]-5- cyanobenzoic acid |
| 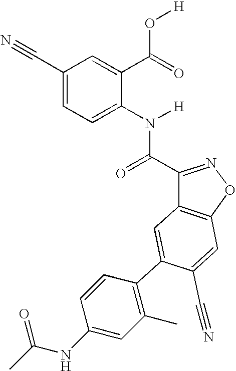 | 2-[({5-[4-(acetylamino)-2-methylphenyl]-6-cyano-1,2- benzisoxazol-3-yl}carbonyl)amino]-5-cyanobenzoic acid |
| 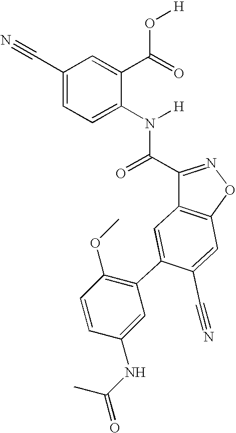 | 2-[({5-[5-(acetylamino)-2-methoxyphenyl]-6-cyano-1,2- benzisoxazol-3-yl}carbonyl)amino]-5-cyanobenzoic acid |
| 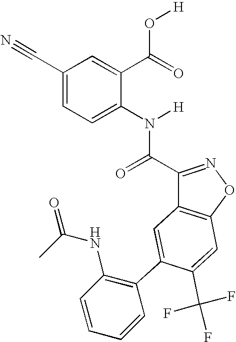 | 2-({[5-[2-(acetylamino)phenyl]-6-(trifluoromethyl)-1,2- benzisoxazol-3-yl]carbonyl}amino)-5-cyanobenzoic acid |
| 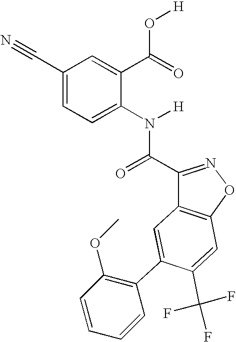 | 5-cyano-2-({[5-(2-methoxyphenyl)-6-(trifluoromethyl)-1,2- benzisoxazol-3-yl]carbonyl}amino)benzoic acid |
| 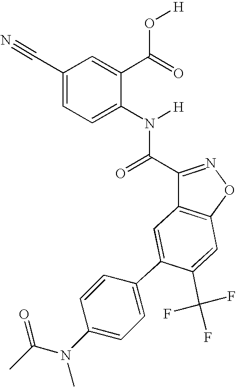 | 2-({[5-{4-[acetyl(methyl)amino]phenyl}-6- (trifluoromethyl)-1,2-benzisoxazol-3-yl]carbonyl]amino)-5- cyanobenzoic acid |
| 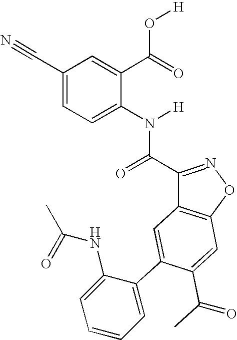 | 2-[({6-acetyl-5-[2-(acetylamino)phenyl]-1,2- benzisoxazol-3-yl}carbonyl)amino]-5-cyanobenzoic acid |
| 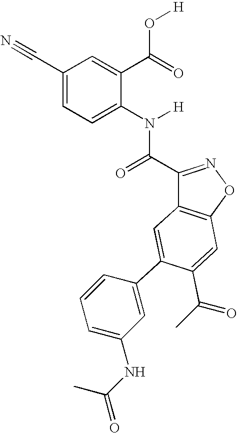 | 2-[({6-acetyl-5-[3-(acetylamino)phenyl]-1,2- benzisoxazol-3-yl}carbonyl)amino]-5-cyanobenzoic acid |
| 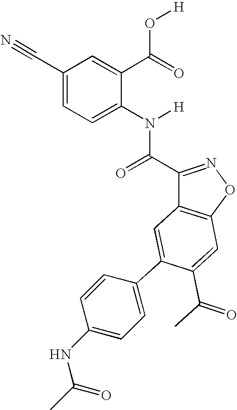 | 2-[({6-acetyl-5-[4-(acetylamino)phenyl]-1,2- benzisoxazol-3-yl}carbonyl)amino]-5-cyanobenzoic acid |
| 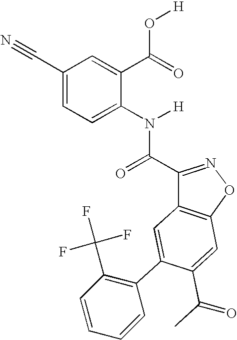 | 2-[({6-acetyl-5-[2-(trifluoromethyl)phenyl]-1,2- benzisoxazol-3-yl}carbonyl)amino]-5-cyanobenzoic acid |
| 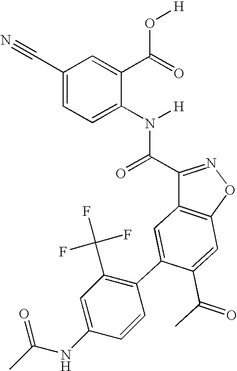 | 2-[({6-acetyl-5-[4-(acetylamino)-2-(trifluoromethyl) phenyl]-1,2-benzisoxazol-3-yl}carbonyl)amino]- 5-cyanobenzoic acid |
| 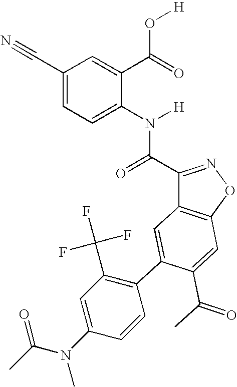 | 2-[({6-acetyl-5-[4-[acetyl(methyl)aminol-2- (trifluoromethyl)phenyl]-1,2-benzisoxazol-3- yl}carbonyl)amino]-5-cyanobenzoic acid |
| 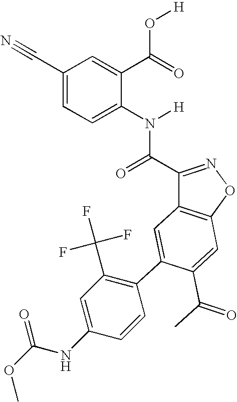 | 2-[({6-acetyl-5-[4-[(methoxycarbonyl)amino]-2- (trifluoromethyl)phenyl]-1,2-benzisoxazol-3- yl}carbonyl)amino]-5-cyanobenzoic acid |
| 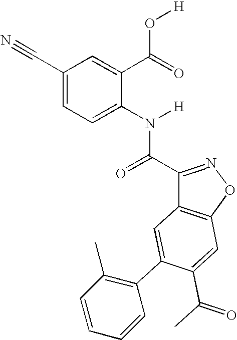 | 2-({[6-acetyl-5-(2-methylphenyl)-1,2-benzisoxazol-3- yl]carbonyl}amino)-5-cyanobenzoic acid |
| 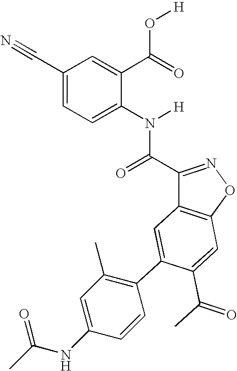 | 2-[({6-acetyl-5-[4-(acetylamino)-2-methylphenyl]- 1,2-benzisoxazol-3-yl}carbonyl)amino]-5- cyanobenzoic acid |
| 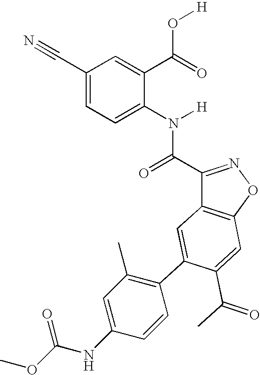 | 2-{[(6-acetyl-5-{4-[(methoxycarbonyl)amino]-2- methylphenyl}-1,2-benzisoxazol-3-yl)carbonyl]amino}- 5-cyanobenzoic acid |
| 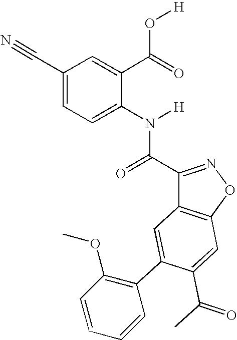 | 2-({[6-acetyl-5-(2-methoxyphenyl)-1,2-benzisoxazol-3- yl]carbonyl}amino)-5-cyanobenzoic acid |
| 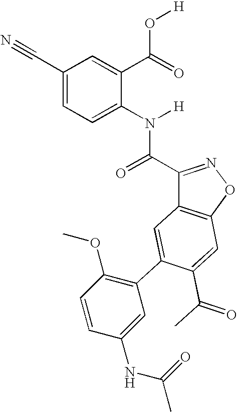 | 2-[({6-acetyl-5-[5-(acetylamino)-2-methoxyphenyl]-1,2- benzisoxazol-3-yl}carbonyl)aminol-5-cyanobenzoic acid |
| 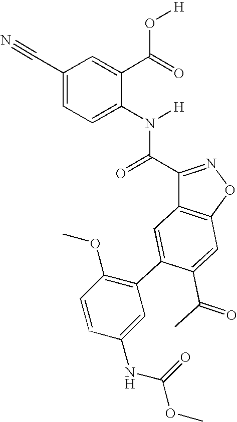 | 2-{[(6-acetyl-5-{2-methoxy-5-[(methoxycarbonyl) amino]phenyl}-1,2-benzisoxazol-3-yl) carbonyl]amino}-5-cyanobenzoic acid |
| 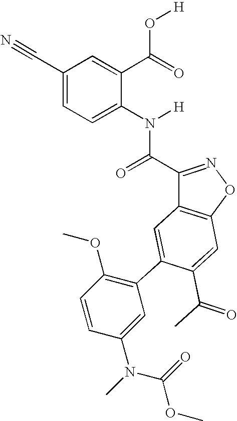 | 2-{[(6-acetyl-5-{2-methoxy-5-[(methoxycarbonyl) (methyl)amino]phenyl}-1,2-benzisoxazol-3-yl) carbonyl]amino}-5-cyanobenzoic acid |
| 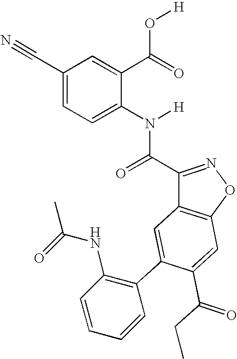 | 2-[({5-[2-(acetylamino)phenyl]-6-propionyl- 1,2-benzisoxazol-3-yl}carbonyl)amino]-5- cyanobenzoic acid |
| 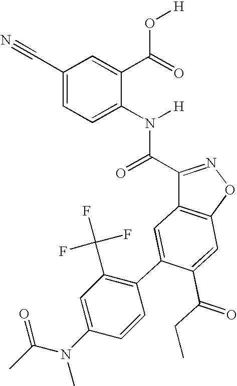 | 2-[({5-[4-[acetyl(methyl)amino]-2- (trifluoromethyl)phenyl]-6-propionyl-1,2-benzisoxazol-3- yl}carbonyl)amino]-5-cyanobenzoic acid |
| 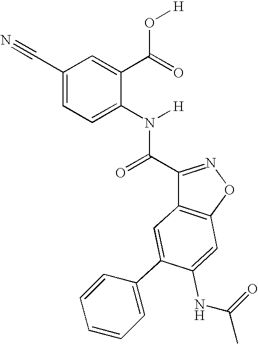 | 2-({[6-(acetylamino)-5-phenyl-1,2-benzisoxazol-3- yl]carbonyl}amino)-5-cyanobenzoic acid |
| 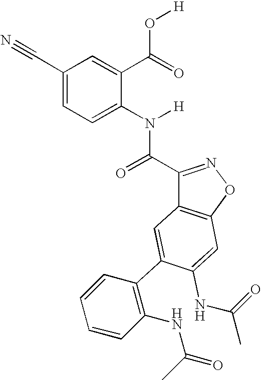 | 2-[({6-(acetylamino)-5-[2-(acetylamino)phenyl]- 1,2-benzisoxazol-3-yl}carbonyl)aminol- 5-cyanobenzoic acid |
| 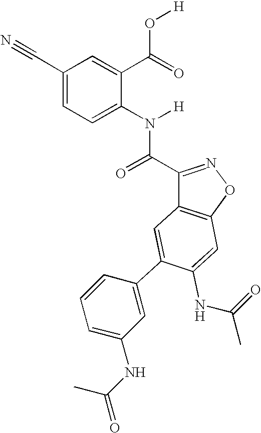 | 2-[({6-(acetylamino)-5-[3-(acetylamino)phenyl]- 1,2-benzisoxazol-3-yl}carbonyl)amino]-5- cyanobenzoic acid |
| 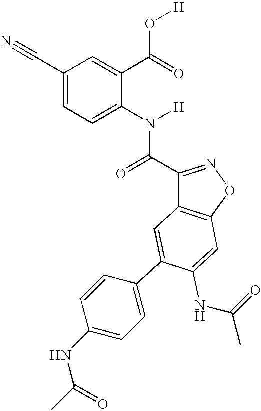 | 2-[({6-(acetylamino)-5-[4-(acetylamino)phenyl]- 1,2-benzisoxazol-3-yl}carbonyl)amino]-5- cyanobenzoic acid |
| 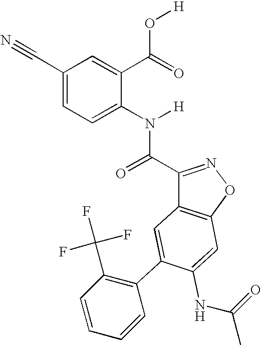 | 2-[({6-(acetylamino)-5-[2-(trifluoromethyl)phenyl]- 1,2-benzisoxazol-3-yl}carbonyl)amino]-5-cyanobenzoic acid |
| 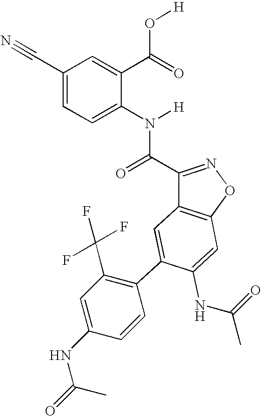 | 2-[({6-(acetylamino)-5-[4-(acetylamino)-2- (trifluoromethyl)phenyl]-1,2-benzisoxazol-3-yl}carbonyl) amino]-5-cyanobenzoic acid |
| 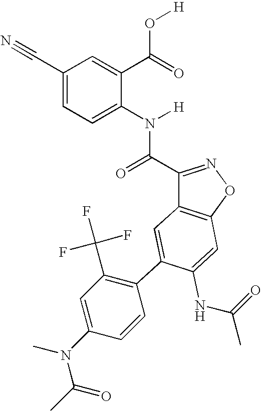 | 2-[({6-(acetylamino)-5-[4-[acetyl(methyl)amino]-2- (trifluoromethyl)phenyl]-1,2-benzisoxazol-3- yl}carbonyl)amino]-5-cyanobenzoic acid |
| 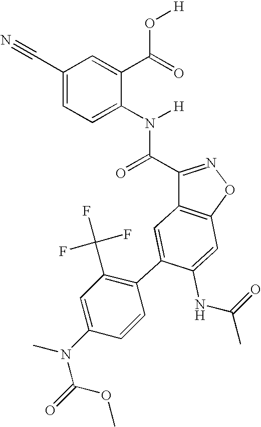 | 2-[({6-(acetylamino)-5-[4-[(methoxycarbonyl)(methyl) amino]-2-(trifluoromethyl)phenyl]-1,2-benzisoxazol-3- yl}carbonyl)amino]-5-cyanobenzoic acid |
| 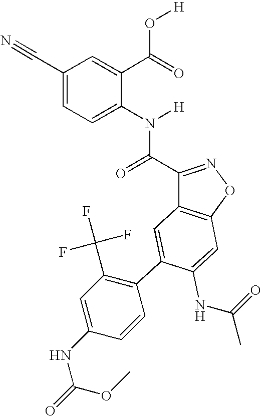 | 2-[({6-(acetylamino)-5-[4-[(methoxycarbonyl)amino]- 2-(trifluoromethyl)phenyl]-1,2-benzisoxazol-3- yl}carbonyl)amino]-5-cyanobenzoic acid |
| 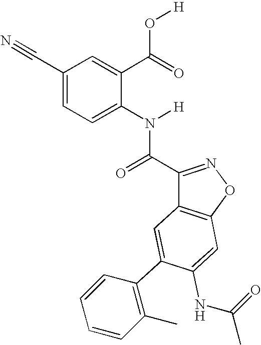 | 2-({[6-(acetylamino)-5-(2-methylphenyl)-1,2-benzisoxazol- 3-yl]carbonyl}amino)-5-cyanobenzoic acid |
| 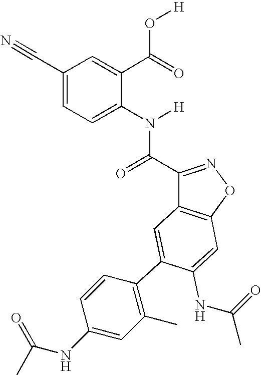 | 2-[({6-(acetylamino)-5-[4-(acetylamino)-2- methylphenyl]-1,2-benzisoxazol-3-yl}carbonyl)amino]-5-cyanobenzoic acid |
| 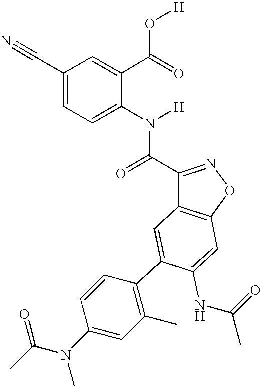 | 2-{[(6-(acetylamino)-5-{4-[acetyl(methyl) amino]-2-methylphenyl}-1,2-benzisoxazol-3-yl) carbonyl]amino}-5-cyanobenzoic acid |
| 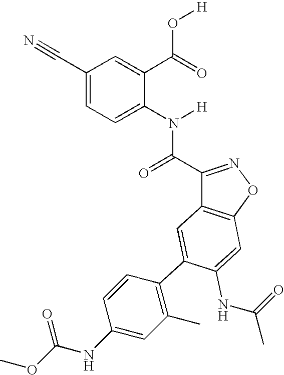 | 2-{[(6-(acetylamino)-5-{4-[(methoxycarbonyl) amino]-2-methylphenyl}-1,2-benzisoxazol-3-yl) carbonyl]amino}-5-cyanobenzoic acid |
| 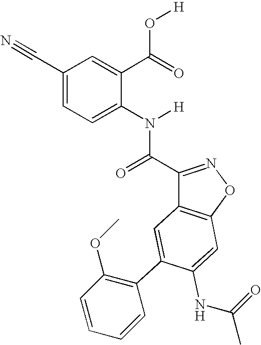 | 2-({[6-(acetylamino)-5-(2-methoxyphenyl)-1,2- benzisoxazol-3-yl]carbonyl}amino)-5- cyanobenzoic acid |
| 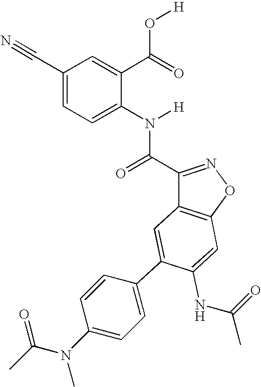 | 2-{[(6-(acetylamino)-5-{4-[acetyl(methyl)amino]phenyl}-1,2-benzisoxazol-3-yl)carbonyl]amino}-5- cyanobenzoic acid |
| 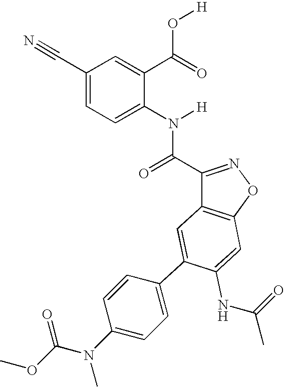 | 2-{[(6-(acetylamino)-5-{4-[(methoxycarbonyl) (methyl)amino]phenyl}-1,2-benzisoxazol-3-yl)carbonyl]amino}-5-cyanobenzoic acid |
| 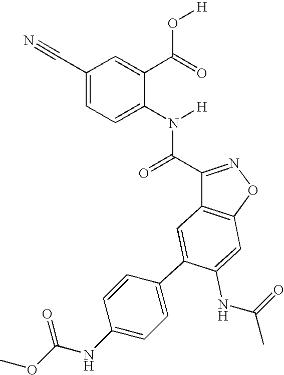 | 2-{[(6-(acetylamino)-5-{4-[(methoxycarbonyl) amino]phenyl}-1,2-benzisoxazol-3-yl)carbonyl]amino}-5-cyanobenzoic acid |
| 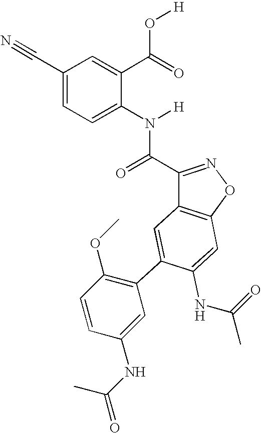 | 2-[({6-(acetylamino)-5-[5-(acetylamino)-2- methoxyphenyl]-1,2-benzisoxazol-3-yl}carbonyl) amino]-5-cyanobenzoic acid |
| 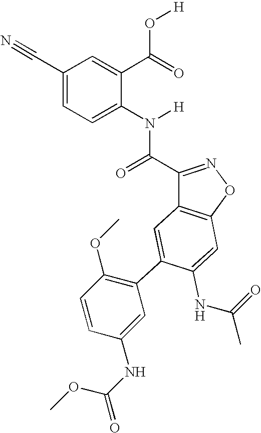 | 2-{[(6-(acetylamino)-5-{2-methoxy-5- [(methoxycarbonyl)amino]phenyl}-1,2-benzisoxazol- 3-yl)carbonyl]amino}-5-cyanobenzoic acid |
| 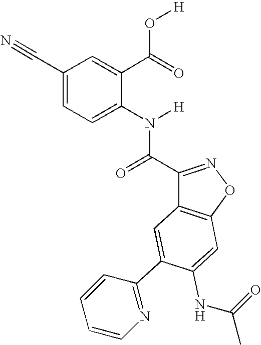 | 2-({[6-(acetylamino)-5-pyridin-2-yl-1,2- benzisoxazol-3-yl]carbonyl}amino)-5-cyanobenzoic acid |
| 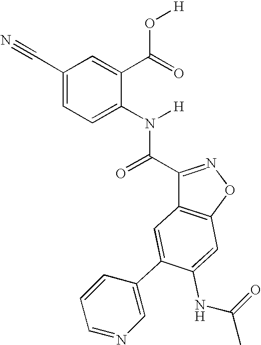 | 2-({[6-(acetylamino)-5-pyridin-3-yl-1,2- benzisoxazol-3-yl]carbonyl}amino)-5-cyanobenzoic acid |
| 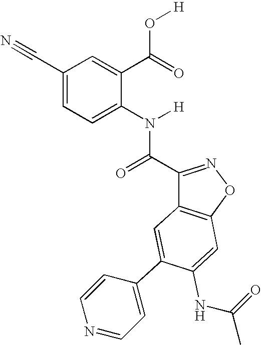 | 2-({[6-(acetylamino)-5-pyridin-4-yl-1,2- benzisoxazol-3-yl]carbonyl}amino)-5-cyanobenzoic acid |
| 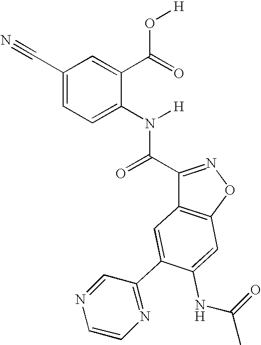 | 2-({[6-(acetylamino)-5-pyrazin-2-yl-1,2- benzisoxazol-3-yl]carbonyl}amino)-5-cyanobenzoic acid |
| 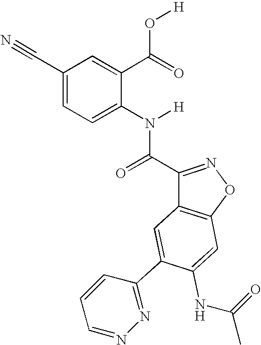 | 2-({[6-(acetylamino)-5-pyridazin-3-yl-1,2- benzisoxazol-3-yl]carbonyl}amino)-5-cyanobenzoic acid |
| 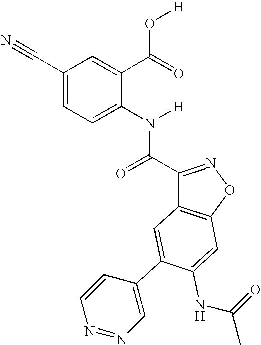 | 2-({[6-(acetylamino)-5-pyridazin-4-yl-1,2- benzisoxazol-3-yl]carbonyl}amino)-5-cyanobenzoic acid |
| 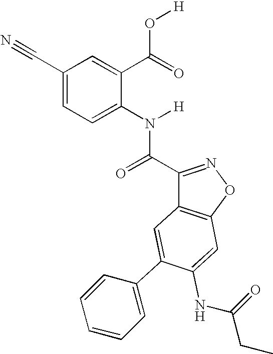 | 5-cyano-2-({[5-phenyl-6-(propionylamino)-1,2- benzisoxazol-3-yl]carbonyl}amino)benzoic acid |
| 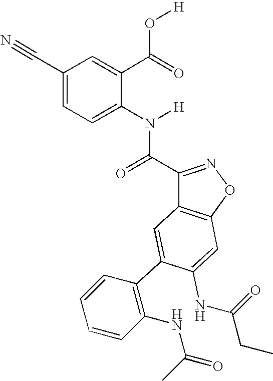 | 2-({[5-[2-(acetylamino)phenyl]-6- (propionylamino)-1,2-benzisoxazol-3-yl]carbonyl}amino)-5-cyanobenzoic acid |
| 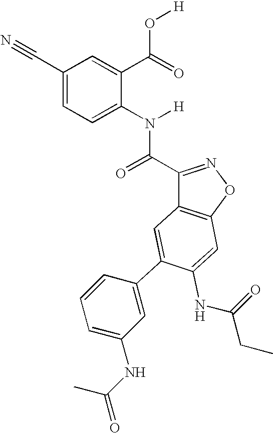 | 2-({[5-[3-(acetylamino)phenyl]-6- (propionylamino)-1,2-benzisoxazol-3-yl]carbonyl}amino)-5-cyanobenzoic acid |
| 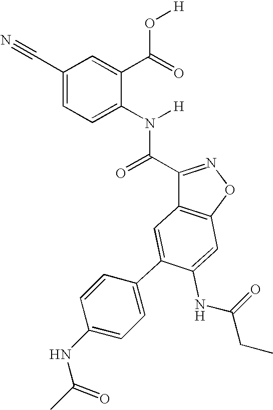 | 2-({[5-[4-(acetylamino)phenyl]-6- (propionylamino)-1,2-benzisoxazol-3-yl]carbonyl}amino)-5-cyanobenzoic acid |
| 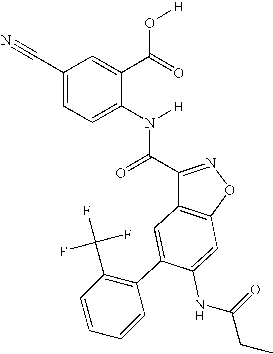 | 5-cyano-2-[({6-(propionylamino)-5-[2- (trifluoromethyl)phenyl]-1,2-benzisoxazol-3- yl}carbonyl)amino]benzoic acid |
| 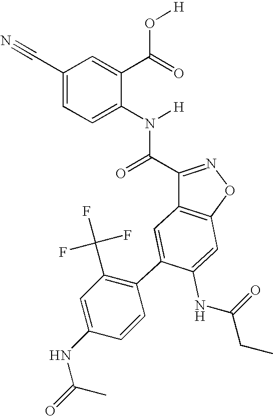 | 2-({[5-[4-(acetylamino)-2-(trifluoromethyl)phenyl]-6- (propionylamino)-1,2-benzisoxazol-3-yl]carbonyl}amino)-5-cyanobenzoic acid |
| 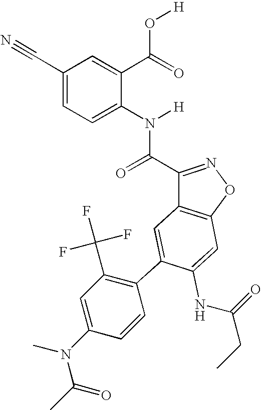 | 2-({[5-[4-[acetyl(methyl)amino]-2- (trifluoromethyl)phenyl]-6-(propionylamino)-1,2- benzisoxazol-3-yl]carbonyl}amino)-5-cyanobenzoic acid |
| 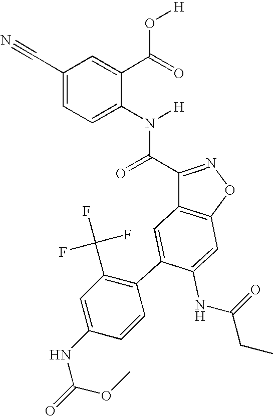 | 5-cyano-2-({[5-[4-[(methoxycarbonyl)amino]-2- (trifluoromethyl)phenyl]-6-(propionylamino)-1,2- benzisoxazol-3-yl]carbonyl}amino)benzoic acid |
| 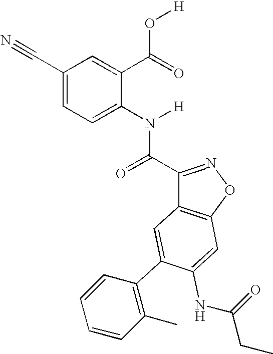 | 5-cyano-2-({[5-(2-methylphenyl)-6-(propionylamino)- 1,2-benzisoxazol-3-yl]carbonyl}amino)benzoic acid |
| 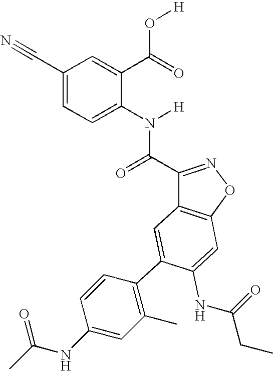 | 2-({[5-[4-(acetylamino)-2-methylphenyl]-6- (propionylamino)-1,2-benzisoxazol-3-yl]carbonyl}amino)-5-cyanobenzoic acid |
| 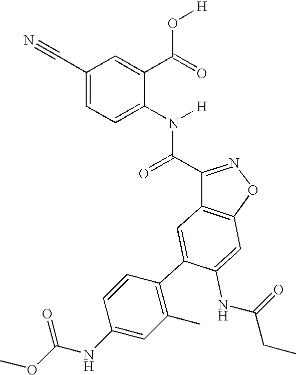 | 5-cyano-2-({[5-{4-[(methoxycarbonyl)amino]-2- methylphenyl}-6-(propionylamino)-1,2-benzisoxazol-3- yl]carbonyl}amino)benzoic acid |
| 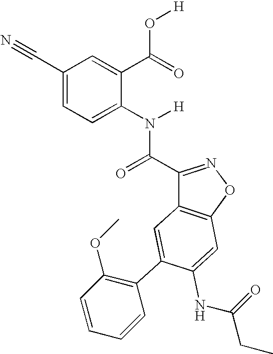 | 5-cyano-2-({[5-(2-methoxyphenyl)-6-(propionylamino)- 1,2-benzisoxazol-3-yl]carbonyl}amino)benzoic acid |
| 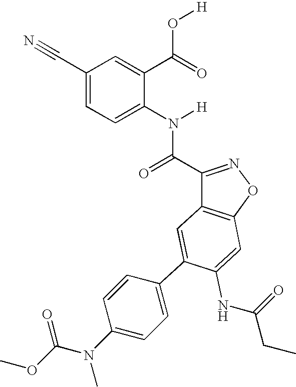 | 5-cyano-2-({[5-{4-[(methoxycarbonyl)(methyl) amino]phenyl}-6-(propionylamino)-1,2-benzisoxazol-3- yl]carbonyl}amino)benzoic acid |
| 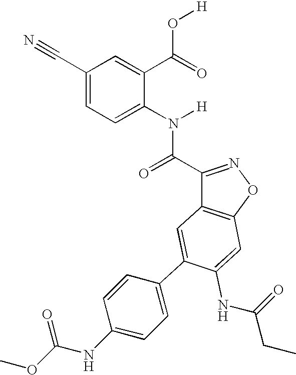 | 5-cyano-2-({[5-{4-[(methoxycarbonyl)amino]phenyl}-6-(propionylamino)-1,2-benzisoxazol- 3-yl]carbonyl}amino)benzoic acid |
| 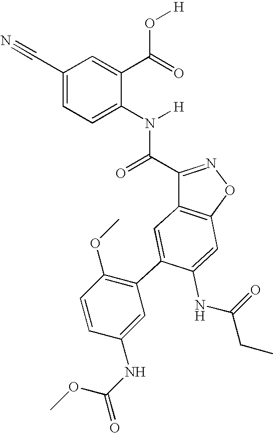 | 5-cyano-2-({[5-{2-methoxy-5-[(methoxycarbonyl)amino]phenyl}-6-(propionylamino)-1,2-benzisoxazol-3- yl]carbonyl}amino)benzoic acid |
| 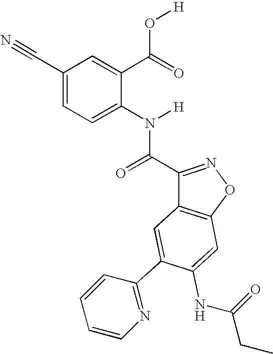 | 5-cyano-2-({[6-(propionylamino)-5-pyridin-2-yl-1,2- benzisoxazol-3-yl]carbonyl}amino)benzoic acid |
| 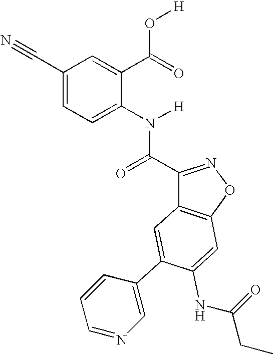 | 5-cyano-2-({[6-(propionylamino)-5-pyridin-3-yl-1,2- benzisoxazol-3-yl]carbonyl}amino)benzoic acid |
| 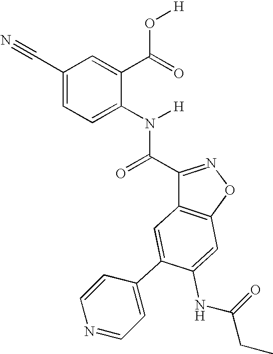 | 5-cyano-2-({[6-(propionylamino)-5-pyridin-4-yl-1,2- benzisoxazol-3-yl]carbonyl}amino)benzoic acid |
| 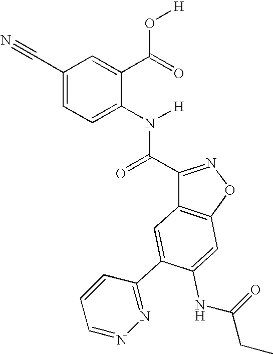 | 5-cyano-2-({[6-(propionylamino)-5-pyridazin-3-yl-1,2- benzisoxazol-3-yl]carbonyl}amino)benzoic acid |
| 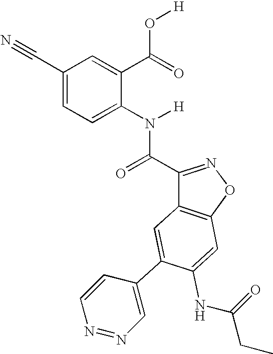 | 5-cyano-2-({[6-(propionylamino)-5-pyridazin-4-yl-1,2- benzisoxazol-3-yl]carbonyl}amino)benzoic acid |
| 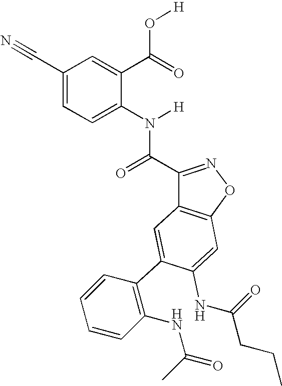 | 2-({[5-[2-(acetylamino)phenyl]-6-(butyrylamino)- 1,2-benzisoxazol-3-yl]carbonyl}amino)-5- cyanobenzoic acid |
| 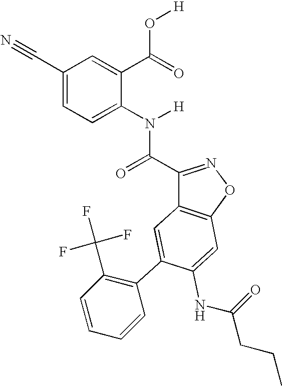 | 2-[({6-(butyrylamino)-5-[2-(trifluoromethyl)phenyl]- 1,2-benzisoxazol-3-yl}carbonyl)amino]-5- cyanobenzoic acid |
| 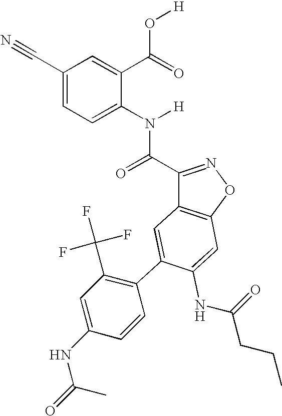 | 2-({[5-[4-(acetylamino)-2-(trifluoromethyl)phenyl]- 6-(butyrylamino)-1,2-benzisoxazol-3-yl]carbonyl}amino)-5-cyanobenzoic acid |
| 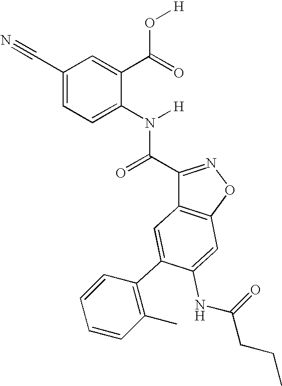 | 2-({[6-(butyrylamino)-5-(2-methylphenyl)-1,2-benzisoxazol- 3-yl]carbonyl}amino)-5-cyanobenzoic acid |
| 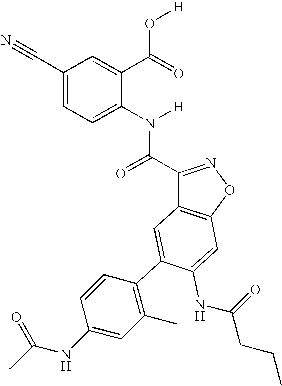 | 2-({[5-[4-(acetylamino)-2-methylphenyl]-6- (butyrylamino)-1,2-benzisoxazol-3-yl]carbonyl}amino)-5-cyanobenzoic acid |
| 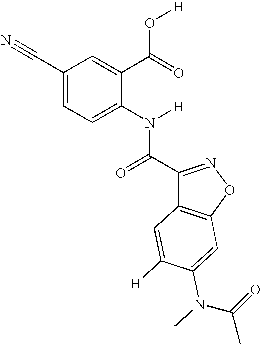 | 2-[({6-[acetyl(methyl)amino]-1,2-benzisoxazol- 3-yl}carbonyl)amino]-5-cyanobenzoic acid |
| 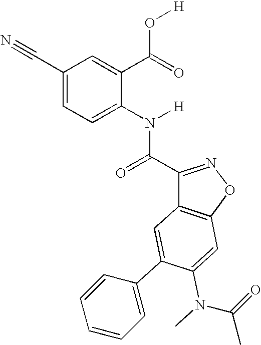 | 2-[({6-[acetyl(methyl)amino]-5-phenyl-1,2- benzisoxazol-3-yl}carbonyl)amino]-5- cyanobenzoic acid |
| 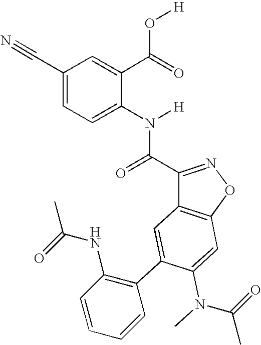 | 2-[({5-[2-(acetylamino)phenyl]-6-[acetyl(methyl) amino]-1,2-benzisoxazol-3-yl}carbonyl)amino]-5- cyanobenzoic acid |
| 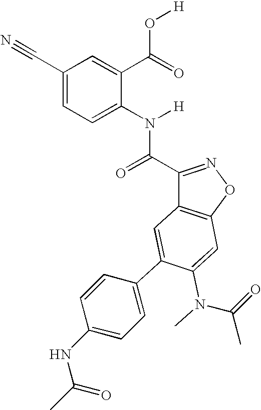 | 2-[({5-[4-(acetylamino)phenyl]-6-[acetyl(methyl) amino]-1,2-benzisoxazol-3-yl}carbonyl)amino]-5- cyanobenzoic acid |
| 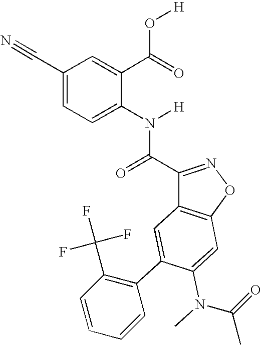 | 2-[({6-[acetyl(methyl)amino]-5-[2-(trifluoromethyl)phenyl]- 1,2-benzisoxazol-3-yl}carbonyl)amino]-5- cyanobenzoic acid |
| 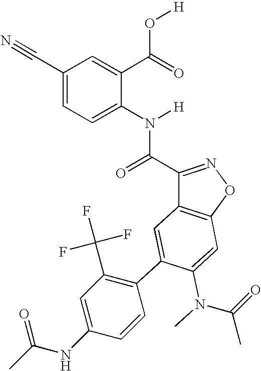 | 2-[({5-[4-(acetylamino)-2-(trifluoromethyl)phenyl]- 6-[acetyl(methyl)amino]-1,2-benzisoxazol-3- yl}carbonyl)amino]-5-cyanobenzoic acid |
| 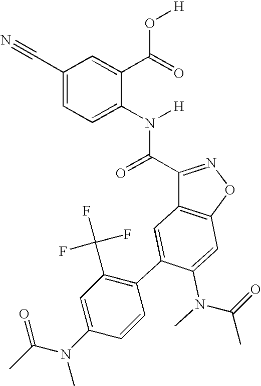 | 2-[({6-[acetyl(methyl)amino]-5-[4-[acetyl(methyl) amino]-2-(trifluoromethyl)phenyl]-1,2- benzisoxazol-3-yl}carbonyl)amino]-5- cyanobenzoic acid |
| 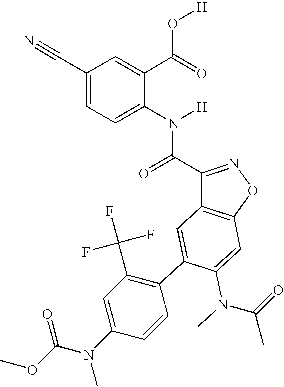 | 2-[({6-[acetyl(methyl)amino]-5-[4-[(methoxycarbonyl) (methyl)amino]-2-(trifluoromethyl)phenyl]-1,2- benzisoxazol-3-yl}carbonyl)amino]-5- cyanobenzoic acid |
| 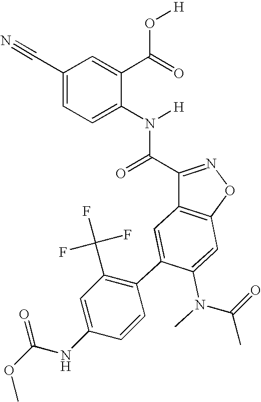 | 2-[({6-[acetyl(methyl)amino]-5-[4-[(methoxycarbonyl) amino]-2-(trifluoromethyl)phenyl]-1,2-benzisoxazol-3- yl}carbonyl)amino]-5-cyanobenzoic acid |
| 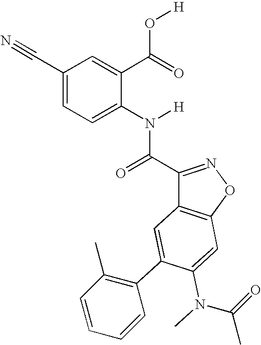 | 2-({[6-[acetyl(methyl)amino]-5-(2-methylphenyl)-1,2- benzisoxazol-3-yl]carbonyl}amino)-5-cyanobenzoic acid |
| 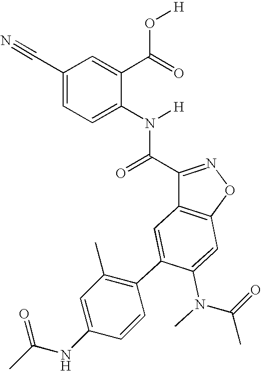 | 2-[({5-[4-(acetylamino)-2-methylphenyl]-6- [acetyl(methyl)amino]-1,2-benzisoxazol-3- yl}carbonyl)amino]-5-cyanobenzoic acid |
| 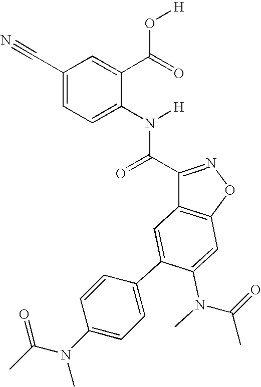 | 2-{[(6-[acetyl(methyl)amino]-5-{4-[acetyl(methyl) amino]phenyl}-1,2-benzisoxazol-3-yl) carbonyl]amino}-5-cyanobenzoic acid |
| 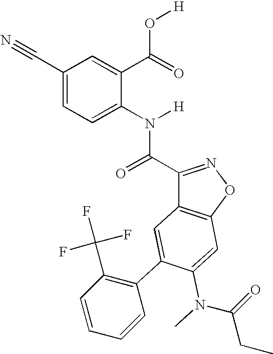 | 5-cyano-2-[({6-[methyl(propionyl)amino]-5-[2- (trifluoromethyl)phenyl]-1,2-benzisoxazol-3-yl}carbonyl)amino]benzoic acid |
| 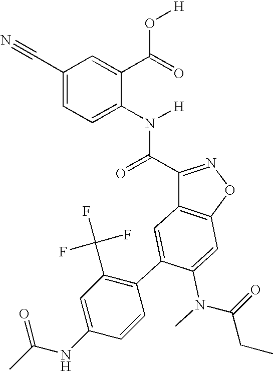 | 2-[({5-[4-(acetylamino)-2-(trifluoromethyl)phenyl]- 6-[methyl(propionyl)amino]-1,2-benzisoxazol- 3-yl]carbonyl)amino]-5-cyanobenzoic acid |
| 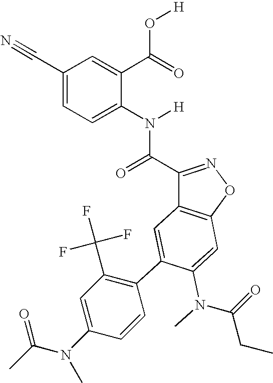 | 2-[({5-[4-[acetyl(methyl)amino]-2- (trifluoromethyl)phenyl]-6-[methyl(propionyl)amino]- 1,2-benzisoxazol-3-yl}carbonyl)amino]-5- cyanobenzoic acid |
| 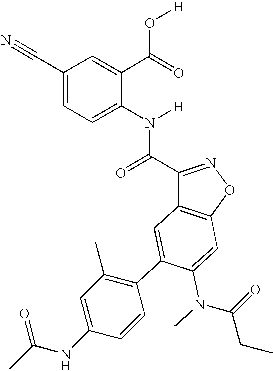 | 2-[({5-[4-(acetylamino)-2-methylphenyl]-6- [methyl(propionyl)amino]-1,2-benzisoxazol-3- yl}carbonyl)amino]-5-cyanobenzoic acid |
| 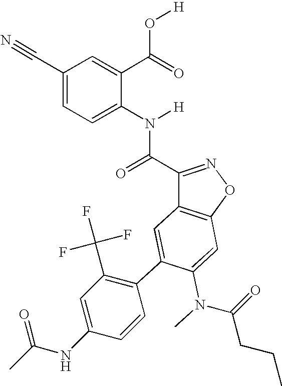 | 2-[({5-[4-(acetylamino)-2-(trifluoromethyl) phenyl]-6-[butyryl(methyl)amino]-1,2-benzisoxazol-3- yl}carbonyl)amino]-5-cyanobenzoic acid |
| 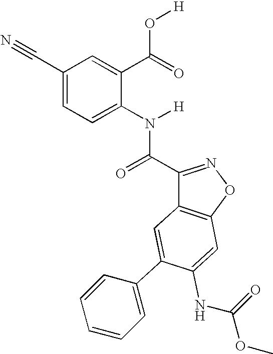 | 5-cyano-2-[({6-[(methoxycarbonyl)amino]-5- phenyl-1,2-benzisoxazol-3-yl}carbonyl)amino]benzoic acid |
| 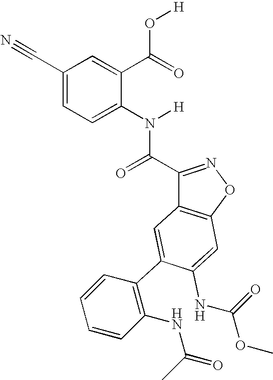 | 2-[({5-[2-(acetylamino)phenyl]-6-[(methoxycarbonyl) amino]-1,2-benzisoxazol-3-yl}carbonyl)amino]- 5-cyanobenzoic acid |
| 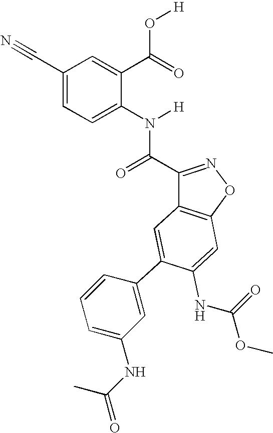 | 2-[({5-[3-(acetylamino)phenyl]-6-[(methoxycarbonyl) amino]-1,2-benzisoxazol-3-yl}carbonyl)amino]- 5-cyanobenzoic acid |
| 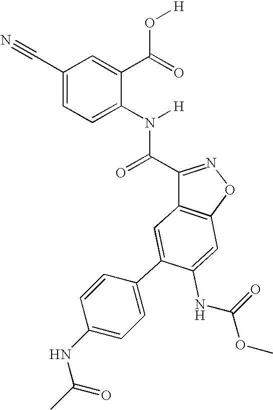 | 2-[({5-[4-(acetylamino)phenyl]-6-[(methoxycarbonyl) amino]-1,2-benzisoxazol-3-yl}carbonyl)amino]- 5-cyanobenzoic acid |
| 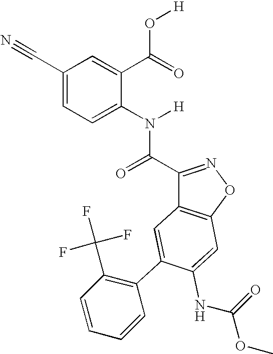 | 5-cyano-2-[({6-[[methoxycarbonyl)amino]-5-[2- (trifluoromethyl)phenyl]-1,2-benzisoxazol-3-yl}carbonyl)amino]benzoic acid |
| 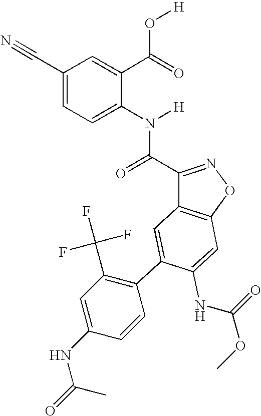 | 2-[({5-[4-(acetylamino)-2-(trifluoromethyl)phenyl]- 6-[(methoxycarbonyl)amino]-1,2-benzisoxazol-3-yl}carbonyl)amino]-5-cyanobenzoic acid |
| 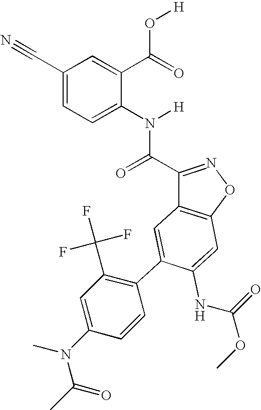 | 2-[({5-[4-[acetyl(methyl)amino]-2-(trifluoromethyl) phenyl]-6-[(methoxycarbonyl)amino]-1,2-benzisoxazol- 3-yl}carbonyl)amino]-5-cyanobenzoic acid |
| 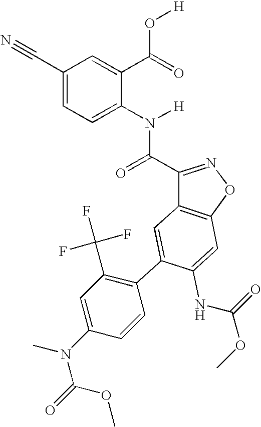 | 5-cyano-2-[({6-[(methoxycarbonyl)amino]-5- [4-[(methoxycarbonyl)(methyl)amino]-2- (trifluoromethyl)phenyl]-1,2-benzisoxazol-3- yl}carbonyl)amino]benzoic acid |
| 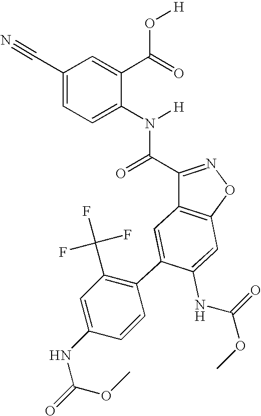 | 5-cyano-2-[({6-[(methoxycarbonyl)amino]-5- [4-[(methoxycarbonyl)amino]-2- (trifluoromethyl)phenyl]-1,2-benzisoxazol-3- yl}carbonyl)amino]benzoic acid |
| 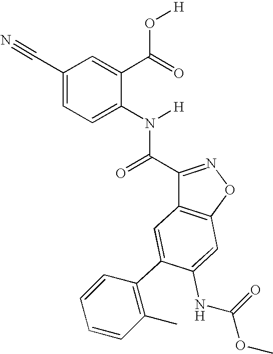 | 5-cyano-2-({[6-[(methoxycarbonyl)amino]-5- (2-methylphenyl)-1,2-benzisoxazol-3-yl]carbonyl}amino)benzoic acid |
| 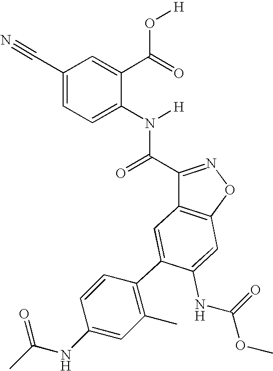 | 2-[({5-[4-(acetylamino)-2-methylphenyl]-6- (methoxycarbonyl)amino]-1,2-benzisoxazol-3-yl}carbonyl)amino]-5-cyanobenzoic acid |
| 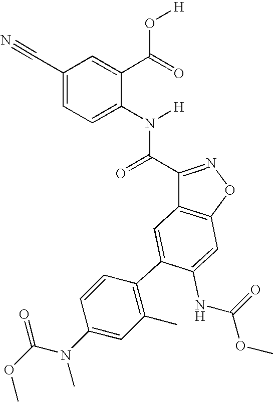 | 5-cyano-2-{[(6-[(methoxycarbonyl)amino]-5- {4-[(methoxycarbonyl)(methyl)amino]-2- methylphenyl}-1,2-benzisoxazol-3-yl)carbonyl]amino}benzoic acid |
| 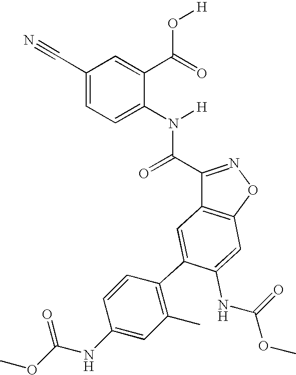 | 5-cyano-2-{[(6-[(methoxycarbonyl)amino]-5- {4-[(methoxycarbonyl)amino]-2-methylphenyl}- 1,2-benzisoxazol-3-yl)carbonyl]amino}benzoic acid |
| 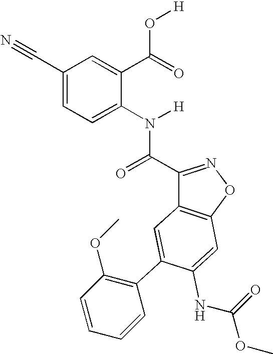 | 5-cyano-2-({[6-[(methoxycarbonyl)amino]-5-(2- methoxyphenyl)-1,2-benzisoxazol-3-yl]carbonyl}amino)benzoic acid |
| 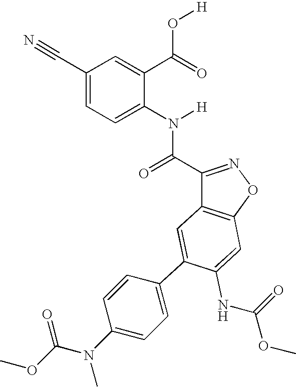 | 5-cyano-2-{[(6-[(methoxycarbonyl)amino]-5-{4- [(methoxycarbonyl)(methyl)amino]phenyl}- 1,2-benzisoxazol-3-yl)carbonyl]amino}benzoic acid |
| 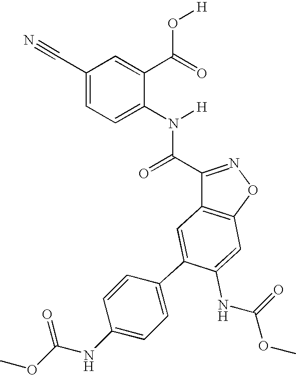 | 5-cyano-2-{[(6-[(methoxycarbonyl)amino]-5-{4- [(methoxycarbonyl)amino]phenyl}-1,2- benzisoxazol-3-yl)carbonyl]amino}benzoic acid |
| 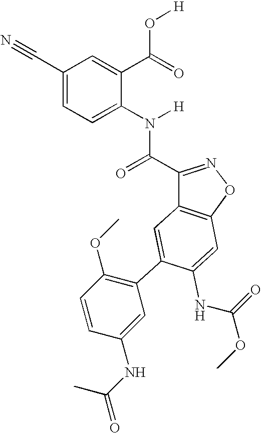 | 2-[({5-[5-(acetylamino)-2-methoxyphenyl]-6- [(methoxycarbonyl)amino]-1,2-benzisoxazol-3-yl}carbonyl)amino]-5-cyanobenzoic acid |
| 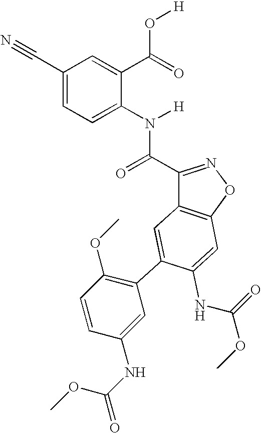 | 5-cyano-2-{[(6-[(methoxycarbonyl)amino]-5-{2-methoxy-5- [(methoxycarbonyl)amino]phenyl}-1,2- benzisoxazol-3-yl)carbonyl]amino}benzoic acid |
| 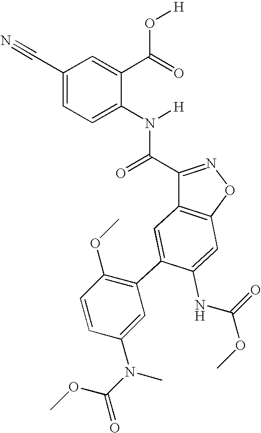 | 5-cyano-2-{[(6-[(methoxycarbonyl)amino]-5-{2-methoxy-5- [(methoxycarbonyl)(methyl)amino]phenyl}- 1,2-benzisoxazol-3-yl)carbonyl]amino}benzoic acid |
| 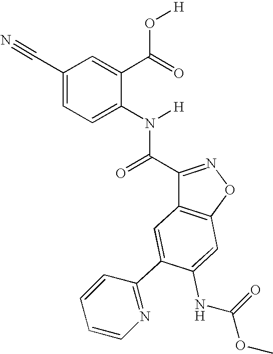 | 5-cyano-2-[({6-[(methoxycarbonyl)amino]-5-pyridin-2-yl- 1,2-benzisoxazol-3-yl}carbonyl)amino]benzoic acid |
| 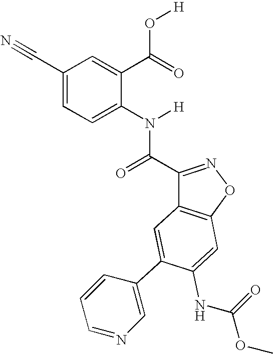 | 5-cyano-2-[({6-[(methoxycarbonyl)amino]-5-pyridin-3-yl- 1,2-benzisoxazol-3-yl}carbonyl)amino]benzoic acid |
| 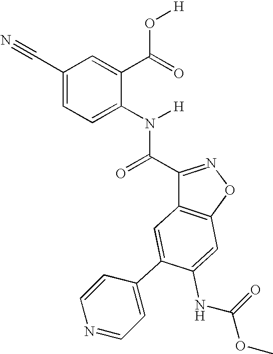 | 5-cyano-2-[({6-[(methoxycarbonyl)amino]-5-pyridin-4-yl- 1,2-benzisoxazol-3-yl}carbonyl)amino]benzoic acid |
| 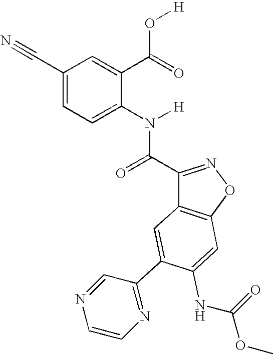 | 5-cyano-2-[({6-[(methoxycarbonyl)amino]-5-pyrazin-2-yl- 1,2-benzisoxazol-3-yl}carbonyl)amino]benzoic acid |
| 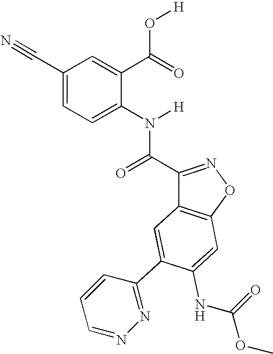 | 5-cyano-2-[({6-[(methoxycarbonyl)amino]-5-pyridazin-3- yl-1,2-benzisoxazol-3-yl)carbonyl)amino]benzoic acid |
| 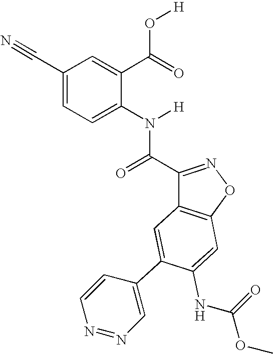 | 5-cyano-2-[({6-[(methoxycarbonyl)amino]-5-pyridazin-4- yl-1,2-benzisoxazol-3-yl}carbonyl)amino]benzoic acid |
| 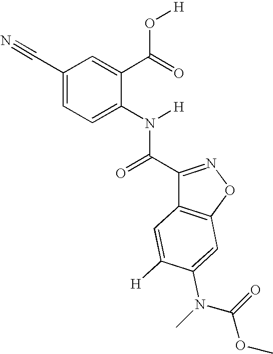 | 5-cyano-2-[({6-[(methoxycarbonyl)(methyl)amino]-1,2- benzisoxazol-3-yl)carbonyl)amino]benzoic acid |
| 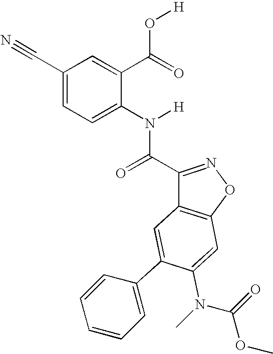 | 5-cyano-2-[({6-[(methoxycarbonyl)(methyl)amino]-5- phenyl-1,2-benzisoxazol-3-yl}carbonyl) amino]benzoic acid |
| 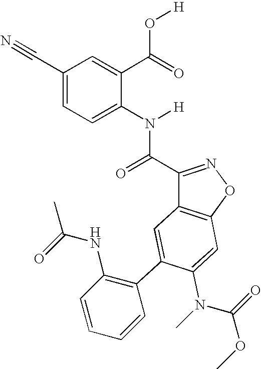 | 2-[({5-[2-(acetylamino)phenyl]-6- [(methoxycarbonyl)(methyl)amino]-1,2- benzisoxazol-3-yl}carbonyl)amino]-5- cyanobenzoic acid |
| 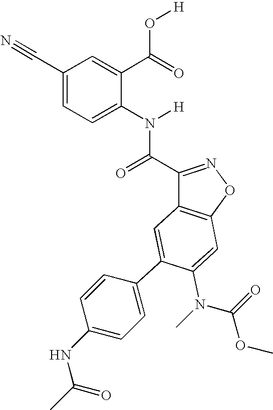 | 2-[({5-[4-(acetylamino)phenyl]-6- [(methoxycarbonyl)(methyl)amino]-1,2- benzisoxazol-3-yl}carbonyl)amino]-5- cyanobenzoic acid |
| 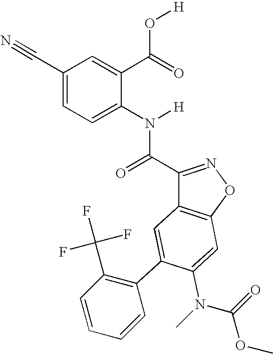 | 5-cyano-2-[({6-[(methoxycarbonyl)(methyl)amino]-5-[2- (trifluoromethyl)phenyl]-1,2-benzisoxazol-3- yl}carbonyl)amino]benzoic acid |
| 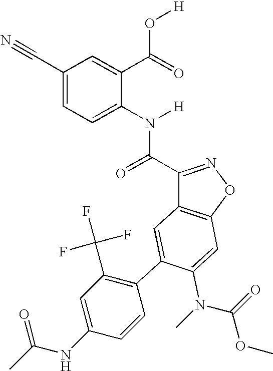 | 2-[({5-[4-(acetylamino)-2-(trifluoromethyl)phenyl]-6- [(methoxycarbonyl)(methyl)amino]-1,2-benzisoxazol-3- yl}carbonyl)amino]-5-cyanobenzoic acid |
| 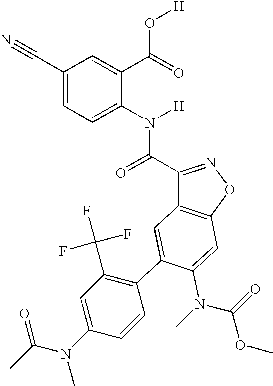 | 2-[({5-[4-[acetyl(methyl)amino]-2-(trifluoromethyl) phenyl]-6-[(methoxycarbonyl)(methyl)amino]- 1,2-benzisoxazol-3-yl}carbonyl)amino]-5- cyanobenzoic acid |
| 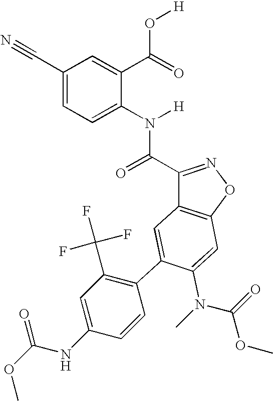 | 5-cyano-2-[({5-[4-[(methoxycarbonyl)amino]-2- (trifluoromethyl)phenyl]-6-[(methoxycarbonyl)(methyl) amino]-1,2-benzisoxazol-3-yl}carbonyl) amino]benzoic acid |
| 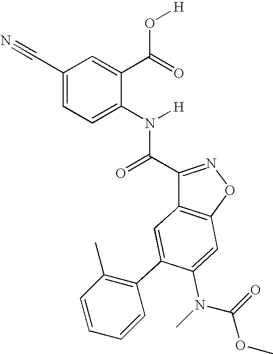 | 5-cyano-2-({[6-[(methoxycarbonyl)(methyl)amino]- 5-(2-methylphenyl)-1,2-benzisoxazol-3- yl]carbonyl}amino)benzoic acid |
| 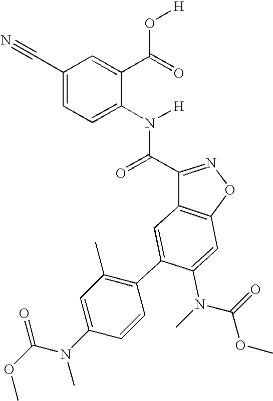 | 5-cyano-2-{[(6-[(methoxycarbonyl)(methyl)amino]- 5-{4-methylphenyl}-1,2-benzisoxazol-3- yl)carbonyl]amino}benzoic acid |
| 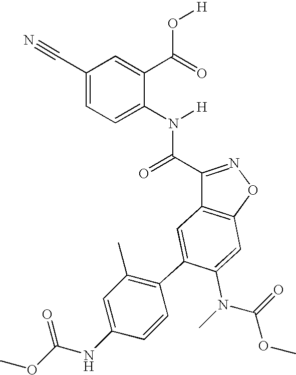 | 5-cyano-2-[({5-{4-[(methoxycarbonyl)amino]-2- methylphenyl}-6-[(methoxycarbonyl)(methyl)amino]-1,2- benzisoxazol-3-yl}carbonyl)amino]benzoic acid |
| 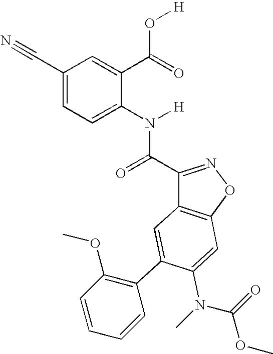 | 5-cyano-2-({[6-[(methoxycarbonyl)(methyl)amino]- 5-(2-methoxyphenyl)-1,2-benzisoxazol-3-yl]carbonyl}amino)benzoic acid |
| 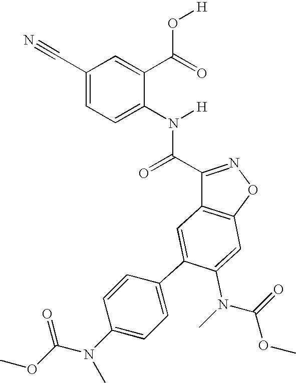 | 5-cyano-2-{[(6-[(methoxycarbonyl)(methyl)amino]- 5-{4-[(methoxycarbonyl)(methyl)amino]phenyl}- 1,2-benzisoxazol-3-yl)carbonyl]amino}benzoic acid |
| 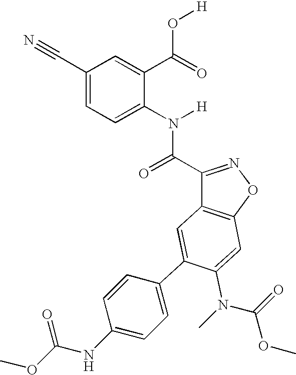 | 5-cyano-2-[({5-{4-[(methoxycarbonyl)amino]phenyl}-6- [(methoxycarbonyl)(methyl)amino]-1,2- benzisoxazol-3-yl}carbonyl)amino]benzoic acid |
| 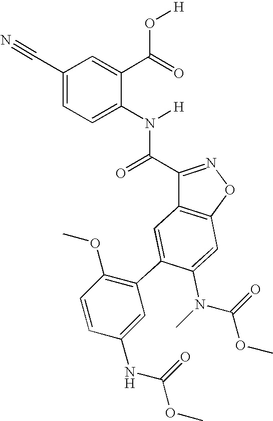 | 5-cyano-2-{[(6-[(methoxycarbonyl)(methyl)amino]-5-{2- methoxy-5-[(methoxycarbonyl)amino]phenyl}- 1,2-benzisoxazol-3-yl)carbonyl]amino}benzoic acid |
| 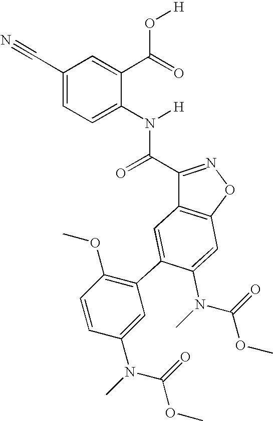 | 5-cyano-2-{[(6-[(methoxycarbonyl)(methyl)amino]-5-{2- methoxy-5-[(methoxycarbonyl)(methyl)amino]phenyl}-1,2-benzisoxazol-3-yl)carbonyl]amino}benzoic acid |
| 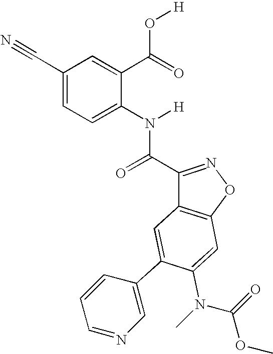 | 5-cyano-2-[({6-[(methoxycarbonyl)(methyl)amino]-5- pyridin-3-yl-1,2-benzisoxazol-3- yl)carbonyl)amino]benzoic acid |
| 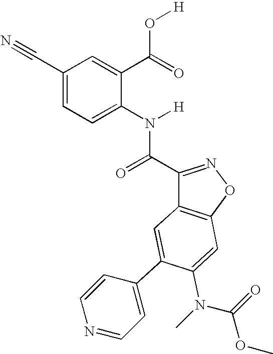 | 5-cyano-2-[({6-[(methoxycarbonyl)(methyl)amino]-5- pyridin-4-yl-1,2-benzisoxazol-3- yl}carbonyl)amino]benzoic acid |
| 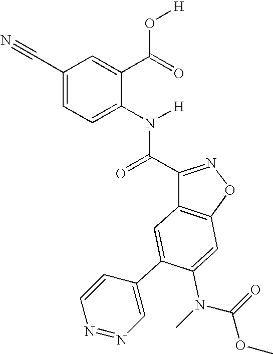 | 5-cyano-2-[({6-[(methoxycarbonyl)(methyl)amino]-5- pyridazin-4-yl-1,2-benzisoxazol-3- yl}carbonyl)amino]benzoic acid |
| 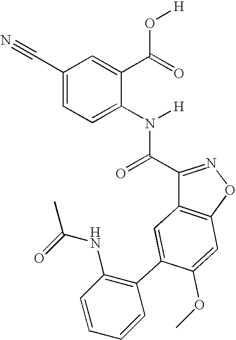 | 2-[({5-[2-(acetylamino)phenyl]-6-methoxy-1,2- benzisoxazol-3-yl}carbonyl)amino]-5- cyanobenzoic acid |
| 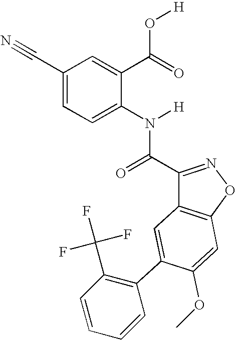 | 5-cyano-2-[({6-methoxy-5-[2-(trifluoromethyl)phenyl]-1,2- benzisoxazol-3-yl}carbonyl)amino]benzoic acid |
| 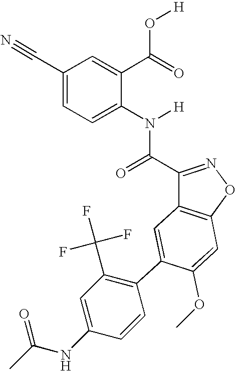 | 2-[({5-[4-(acetylamino)-2-(trifluoromethyl)phenyl]-6-methoxy- 1,2-benzisoxazol-3-yl}carbonyl)amino]-5- cyanobenzoic acid |
| 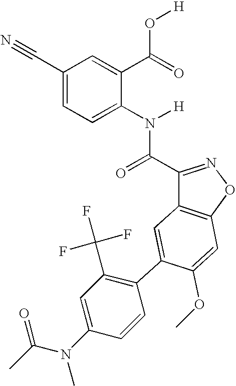 | 2-[({5-[4-[acetyl(methyl)amino]-2-(trifluoromethyl) phenyl]-6-methoxy-1,2-benzisoxazol-3-yl}carbonyl) amino]-5-cyanobenzoic acid |
| 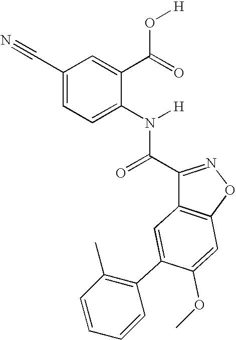 | 5-cyano-2-({[6-methoxy-5-(2-methylphenyl)-1,2- benzisoxazol-3-yl]carbonyl}amino)benzoic acid |
| 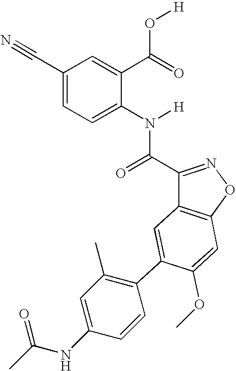 | 2-[({5-[4-(acetylamino)-2-methylphenyl]-6- methoxy-1,2-benzisoxazol-3-yl}carbonyl)amino]-5- cyanobenzoic acid |
| 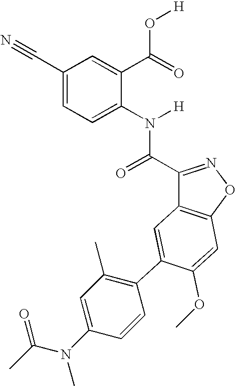 | 2-{[(5-{4-[acetyl(methyl)amino]-2-methylphenyl}-6- methoxy-1,2-benzisoxazol-3-yl)carbonyl]amino}- 5-cyanobenzoic acid |
| 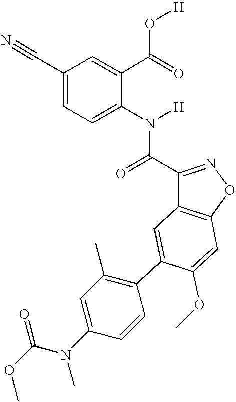 | 5-cyano-2-{[(6-methoxy-5-{4-[(methoxycarbonyl)(methyl) amino]-2-methylphenyl}-1,2-benzisoxazol-3- yl)carbonyl]amino}benzoic acid |
| 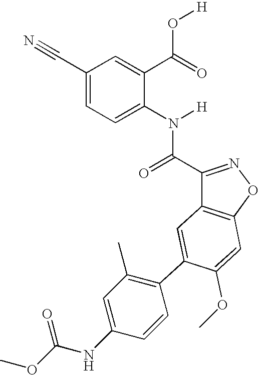 | 5-cyano-2-{[(6-methoxy-5-{4-[(methoxycarbonyl)amino]- 2-methylphenyl}-1,2-benzisoxazol-3-yl) carbonyl]amino}benzoic acid |
| 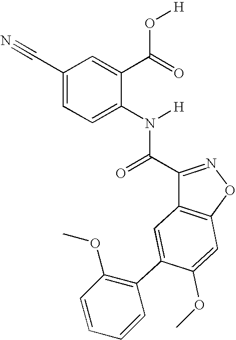 | 5-cyano-2-({[6-methoxy-5-(2-methoxyphenyl)-1,2-benzisoxazol- 3-yl]carbonyl}amino)benzoic acid |
| 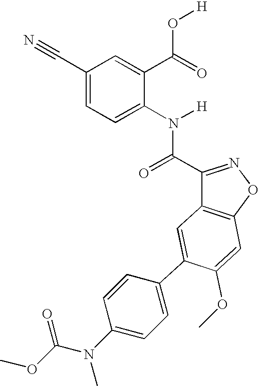 | 5-cyano-2-{[(6-methoxy-5-{4-[(methoxycarbonyl)(methyl) amino]phenyl}-1,2-benzisoxazol-3- yl)carbonyl]amino}benzoic acid |
| 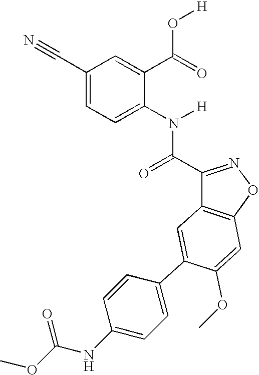 | 5-cyano-2-{[(6-methoxy-5-{4-[(methoxycarbonyl)amino]phenyl}-1,2-benzisoxazol-3-yl)carbonyl]amino}benzoic acid |
| 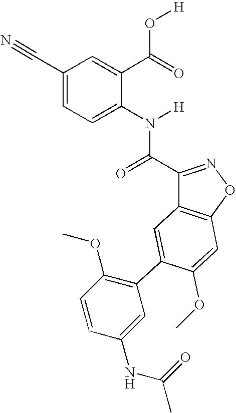 | 2-[({5-[5-(acetylamino)-2-methoxyphenyl]-6-methoxy-1,2- benzisoxazol-3-yl}carbonyl)amino]-5-cyanobenzoic acid |
| 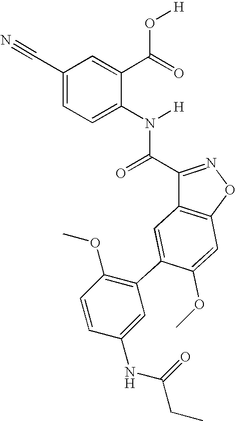 | 5-cyano-2-{[(6-methoxy-5-{2-methoxy-5- [(methoxycarbonyl)amino]phenyl}-1,2- benzisoxazol-3-yl)carbonyl]amino}benzoic acid |
| 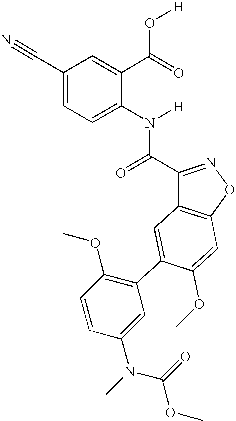 | 5-cyano-2-{[(6-methoxy-5-{2-methoxy-5- [(methoxycarbonyl)(methyl)amino]phenyl}- 1,2-benzisoxazol-3-yl)carbonyl]amino}benzoic acid |
| 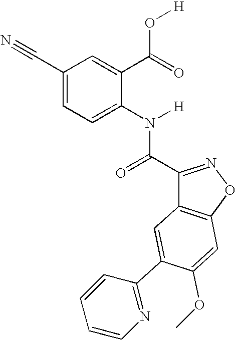 | 5-cyano-2-{[(6-methoxy-5-pyridin-2-yl-1,2- benzisoxazol-3-yl)carbonyl]amino}benzoic acid |
| 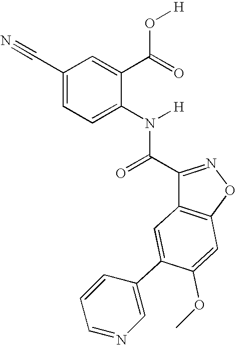 | 5-cyano-2-{[(6-methoxy-5-pyridin-3-yl-1,2- benzisoxazol-3-yl)carbonyl]amino}benzoic acid |
| 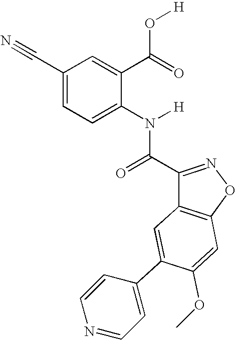 | 5-cyano-2-{[(6-methoxy-5-pyridin-4-yl-1,2- benzisoxazol-3-yl)carbonyl]amino}benzoic acid |
| 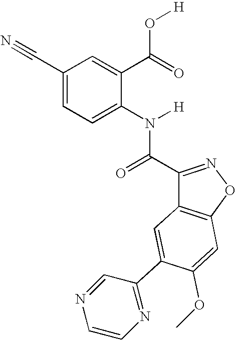 | 5-cyano-2-{[(6-methoxy-5-pyrazin-2-yl-1,2- benzisoxazol-3-yl)carbonyl]amino}benzoic acid |
| 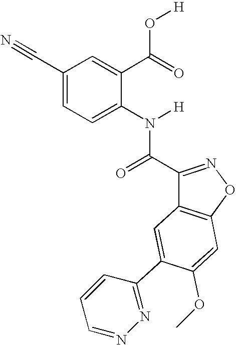 | 5-cyano-2-{[(6-methoxy-5-pyridazin-3-yl-1,2-benzisoxazol- 3-yl)carbonyl]amino}benzoic acid |
| 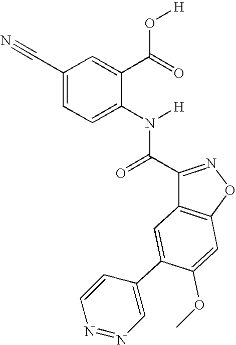 | 5-cyano-2-{[(6-methoxy-5-pyridazin-4-yl-1,2-benzisoxazol- 3-yl)carbonyl]amino}benzoic acid |
| 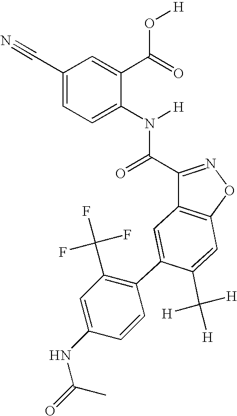 | 2-[({5-[4-(acetylamino)-2-(trifluoromethyl)phenyl]- 6-methyl-1,2-benzisoxazol-3-yl}carbonyl)amino]-5- cyanobenzoic acid |
| 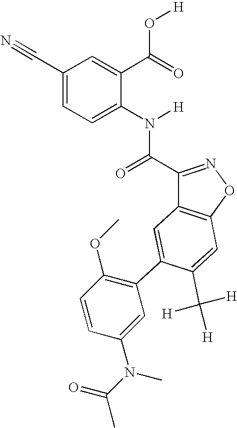 | 2-{[(5-{5-[acetyl(methyl)amino]-2-methoxyphenyl}- 6-methyl-1,2-benzisoxazol-3-yl)carbonyl]amino}-5-cyanobenzoic acid |
| 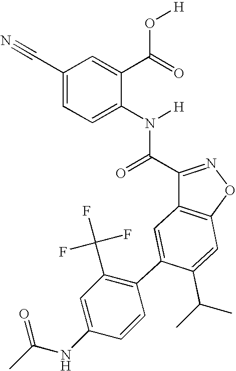 | 2-[({5-[4-(acetylamino)-2-(trifluoromethyl)phenyl]- 6-isopropyl-1,2-benzisoxazol-3-}carbonyl)amino]-5- cyanobenzoic acid |
| 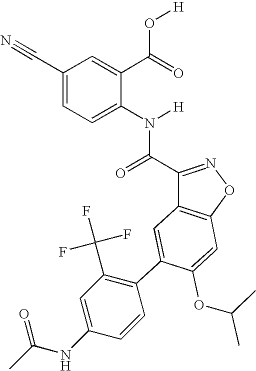 | 2-[({5-[4-(acetylamino)-2-(trifluoromethyl)phenyl]- 6-isopropoxy-1,2-benzisoxazol-3-yl]carbonyl)amino]- 5-cyanobenzoic acid |
| 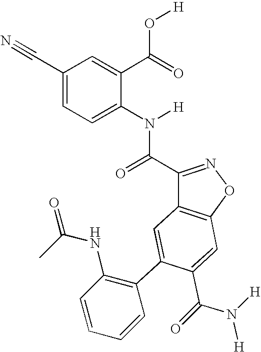 | 2-({[5-[2-(acetylamino)phenyl]-6-(aminocarbonyl)- 1,2-benzisoxazol-3-yl]carbonyl)amino)-5- cyanobenzoic acid |
| 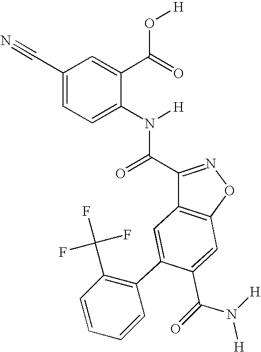 | 2-[({6-(aminocarbonyl)-5-[2-(trifluoromethyl)phenyl]- 1,2-benzisoxazol-3-yl}carbonyl)aminol- 5-cyanobenzoic acid |
| 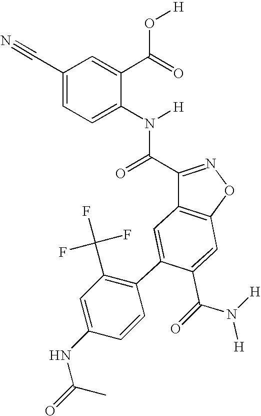 | 2-({[5-[4-(acetylamino)-2-(trifluoromethyl)phenyl]-6- (aminocarbonyl)-1,2-benzisoxazol-3-yl]carbonyl}amino)- 5-cyanobenzoic acid |
| 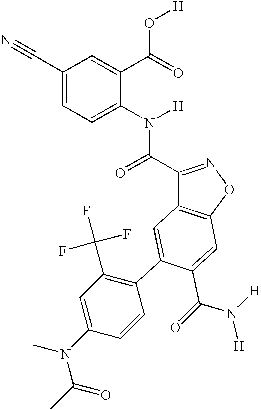 | 2-({[5-[4-[acetyl(methyl)amino]-2- (trifluoromethyl)phenyl]-6-(aminocarbonyl)-1,2- benzisoxazol-3-yl]carbonyl}amino)-5- cyanobenzoic acid |
| 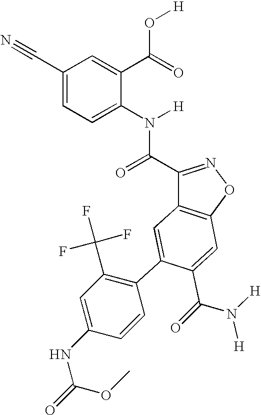 | 2-[({6-(aminocarbonyl)-5-[4-[(methoxycarbonyl)amino]- 2-(trifluoromethyl)phenyl]-1,2-benzisoxazol-3-yl}carbonyl)amino]-5-cyanobenzoic acid |
| 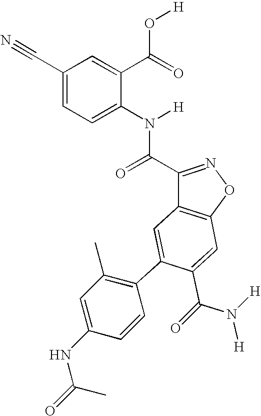 | 2-({[5-[4-(acetylamino)-2-methylphenyl]-6- (aminocarbonyl)-1,2-benzisoxazol-3-yl]carbonyl}amino)-5-cyanobenzoic acid |
| 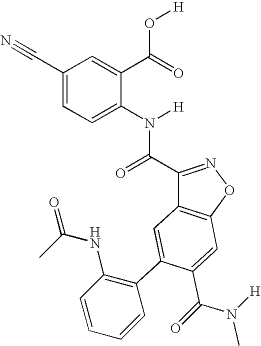 | 2-[({5-[2-(acetylamino)phenyl]-6-[(methylamino) carbonyl]-1,2-benzisoxazol-3-yl}carbonyl) amino]-5-cyanobenzoic acid |
| 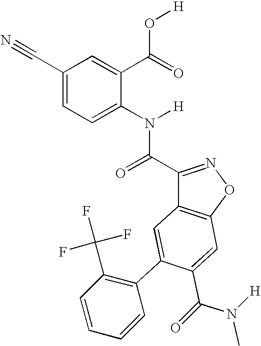 | 5-cyano-2-[({6-[(methylamino)carbonyl]-5-[2- (trifluoromethyl)phenyl]-1,2-benzisoxazol- 3-yl)carbonyl)amino]benzoic acid |
| 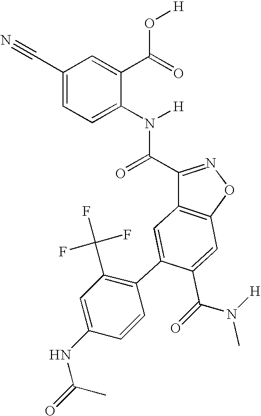 | 2-[({5-[4-(acetylamino)-2-(trifluoromethyl)phenyl]-6- [(methylamino)carbonyl]-1,2-benzisoxazol-3-yl}carbonyl)amino]-5-cyanobenzoic acid |
| 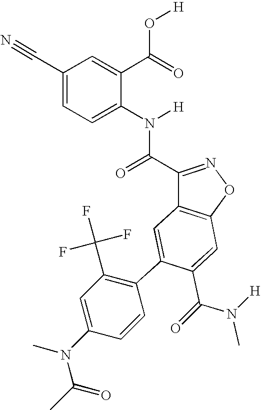 | 2-[({5-[4-[acetyl(methyl)amino]-2-(trifluoromethyl)phenyl]- 6-[(methylamino)carbonyl]-1,2-benzisoxazol-3- yl}carbonyl)amino]-5-cyanobenzoic acid |
| 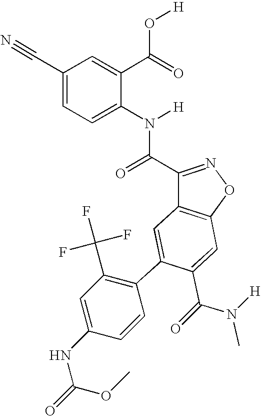 | 5-cyano-2-[({5-[4-[(methoxycarbonyl)amino]-2- (trifluoromethyl)phenyl]-6-[methylamino) carbonyl]-1,2-benzisoxazol-3- yl}carbonyl)amino]benzoic acid |
| 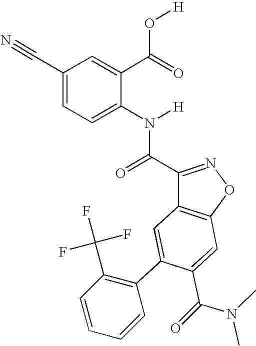 | 5-cyano-2-[({6-[(dimethylamino)carbonyl]-5-[2- (trifluoromethyl)phenyl]-1,2-benzisoxazol-3-yl}carbonyl)amino]benzoic acid |
| 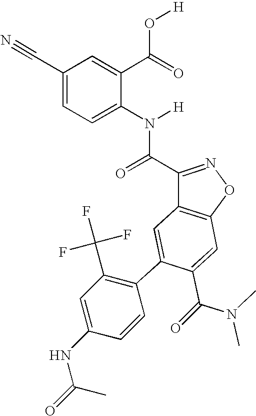 | 2-[({5-[4-(acetylamino)-2-(trifluoromethyl)phenyl]-6- [(dimethylamino)carbonyl]-1,2-benzisoxazol- 3-yl}carbonyl)amino]-5-cyanobenzoic acid |
| 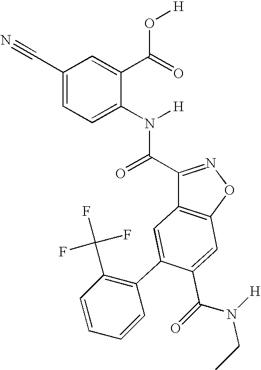 | 5-cyano-2-[({6-[(ethylamino)carbonyl]-5-[2- (trifluoromethyl)phenyl]-1,2-benzisoxazol-3- yl}carbonyl)amino]benzoic acid |
| 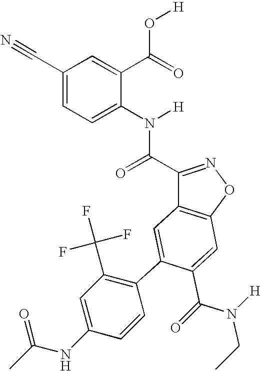 | 2-[({5-[4-(acetylamino)-2-(trifluoromethyl)phenyl]-6- [(ethylamino)carbonyl]-1,2-benzisoxazol-3-yl}carbonyl)amino]-5-cyanobenzoic acid |
| 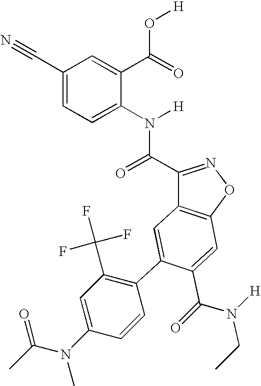 | 2-[({5-[4-[acetyl(methyl)amino]-2- (trifluoromethyl)phenyl]-6-[(ethylamino)carbonyl]-1,2- benzisoxazol-3-yl}carbonyl)amino]-5-cyanobenzoic acid |
| 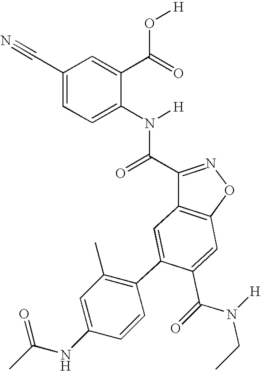 | 2-[({5-[4-(acetylamino)-2-methylphenyl]-6- [(ethylamino)carbonyl]-1,2-benzisoxazol-3-yl}carbonyl)amino]-5-cyanobenzoic acid |
| 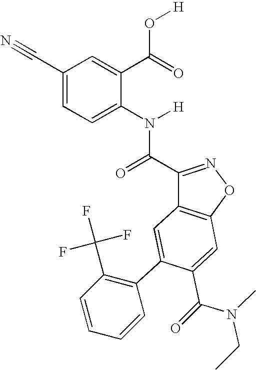 | 5-cyano-2-[({6-{[ethyl(methyl)amino]carbonyl}-5-[2- (trifluoromethyl)phenyl]-1,2-benzisoxazol-3- yl}carbonyl)amino]benzoic acid |
| 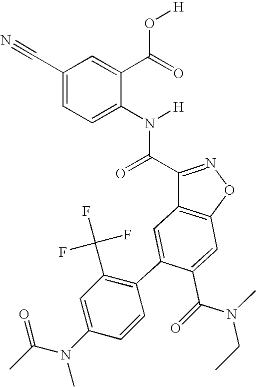 | 2-{[(5-[4-[acetyl(methyl)amino]-2-(trifluoromethyl)phenyl]- 6-{[ethyl(methyl)amino]carbonyl}-1,2- benzisoxazol-3-yl)carbonyl]amino}-5- cyanobenzoic acid |
| 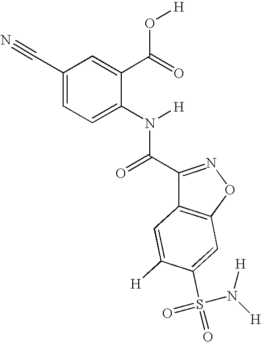 | 2-({[6-(aminosulfonyl)-1,2-benzisoxazol-3-yl]carbonyl}amino)-5-cyanobenzoic acid |
| 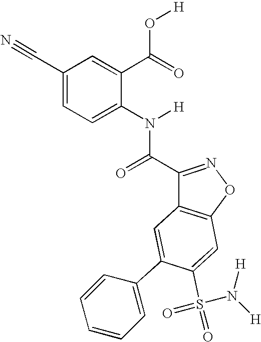 | 2-({[6-(aminosulfonyl)-5-phenyl-1,2-benzisoxazol-3-yl]carbonyl}amino)-5-cyanobenzoic acid |
| 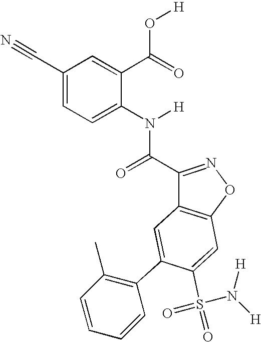 | 2-({[6-(aminosulfonyl)-5-(2-methylphenyl)-1,2- benzisoxazol-3-yl]carbonyl}amino)-5-cyanobenzoic acid |
| 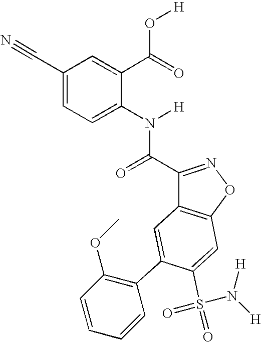 | 2-({[6-(aminosulfonyl)-5-(2-methoxyphenyl)-1,2- benzisoxazol-3-yl]carbonyl}amino)-5-cyanobenzoic acid |
| 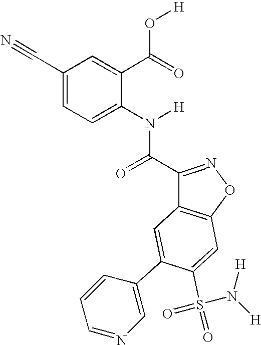 | 2-({[6-(aminosulfonyl)-5-pyridin-3-yl-1,2- benzisoxazol-3-yl]carbonyl}amino)-5-cyanobenzoic acid |
| 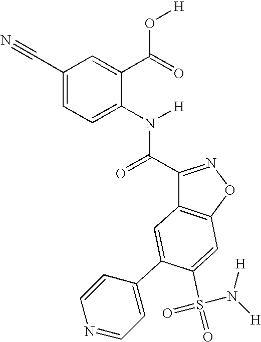 | 2-({[6-(aminosulfonyl)-5-pyridin-4-yl-1,2- benzisoxazol-3-yl]carbonyl}amino)-5-cyanobenzoic acid |
| 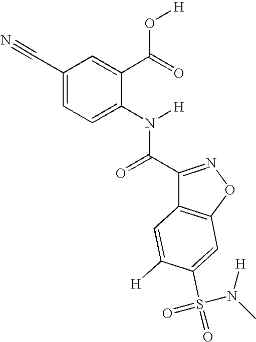 | 5-cyano-2-[({6-[(methylamino)sulfonyl]-1,2- benzisoxazol-3-yl}carbonyl)amino]benzoic acid |
| 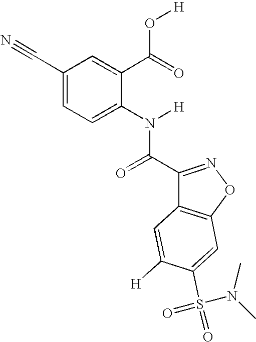 | 5-cyano-2-[({6-[(dimethylamino)sulfonyl]-1,2- benzisoxazol-3-yl}carbonyl)amino]benzoic acid |
| 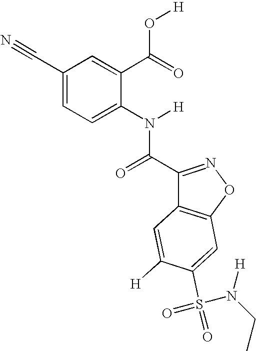 | 5-cyano-2-[({6-[(ethylamino)sulfonyl]-1,2- benzisoxazol-3-yl}carbonyl)amino]benzoic acid |
| 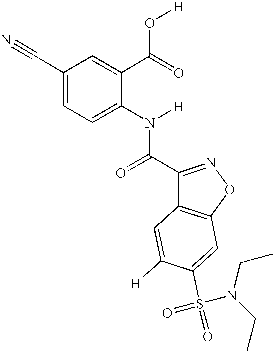 | 5-cyano-2-[({6-[(diethylamino)sulfonyl]-1,2- benzisoxazol-3-yl}carbonyl)amino]benzoic acid |
| 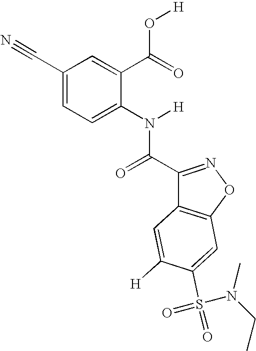 | 5-cyano-2-{[(6-{[ethyl(methyl)amino]sulfonyl}-1,2- benzisoxazol-3-yl)carbonyl]amino}benzoic acid |
| 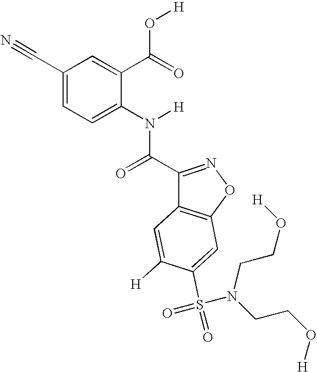 | 2-{[(6-{[bis(2-hydroxyethyl)amino]sulfonyl}-1,2- benzisoxazol-3-yl)carbonyl]amino}-5- cyanobenzoic acid |
| 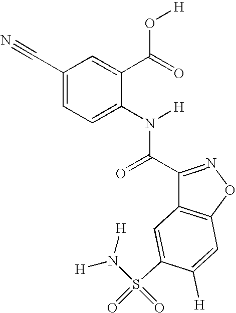 | 2-({[5-(aminosulfonyl)-1,2-benzisoxazol-3-yl]carbonyl}amino)-5-cyanobenzoic acid |
| 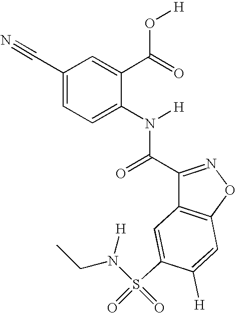 | 5-cyano-2-[({5-[(ethylamino)sulfonyl]-1,2-benzisoxazol- 3-yl]carbonyl)amino]benzoic acid |
| 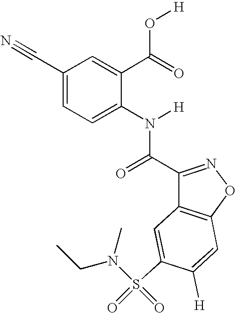 | 5-cyano-2-{[(5-{[ethyl(methyl)amino]sulfonyl}-1,2- benzisoxazol-3-yl)carbonyl]amino}benzoic acid |
| 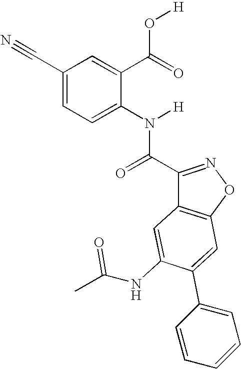 | 2-({[5-(acetylamino)-6-phenyl-1,2-benzisoxazol-3-yl]carbonyl}amino)-5-cyanobenzoic acid |
| 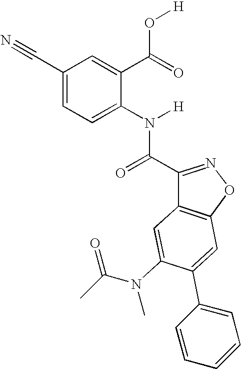 | 2-[({5-[acetyl(methyl)aminol-6-phenyl-1,2- benzisoxazol-3-yl}carbonyl)amino]-5- cyanobenzoic acid |
| 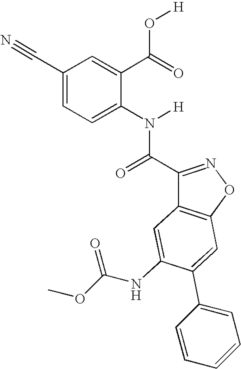 | 5-cyano-2-[({5-[(methoxycarbonyl)amino]-6-phenyl- 1,2-benzisoxazol-3-yl)carbonyl)amino]benzoic acid |
| 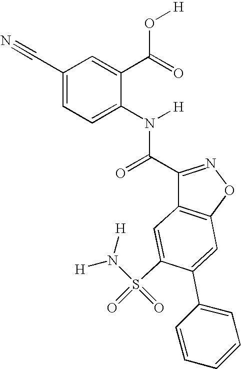 | 2-({[5-(aminosulfonyl)-6-phenyl-1,2-benzisoxazol-3- yl]carbonyl}amino)-5-cyanobenzoic acid |
| 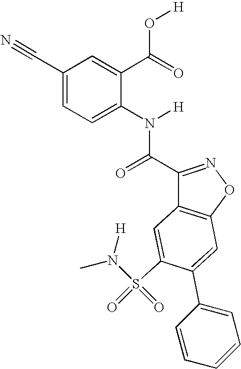 | 5-cyano-2-[({5-[(methylamino)sulfonyl]-6-phenyl- 1,2-benzisoxazol-3-yl}carbonyl)amino]benzoic acid |
| 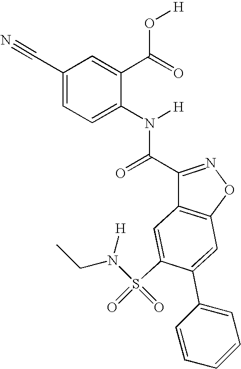 | 5-cyano-2-[({5-[(ethylamino)sulfonyl]-6-phenyl- 1,2-benzisoxazol-3-yl}carbonyl)amino]benzoic acid |
| 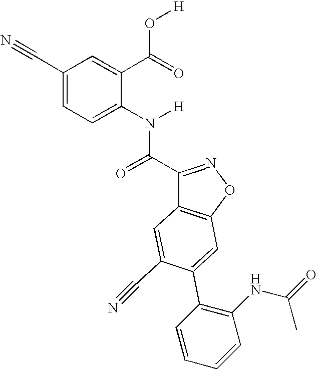 | 2-[({6-[2-(acetylamino)phenyl]-5-cyano-1,2- benzisoxazol-3-yl}carbonyl)amino]-5- cyanobenzoic acid |
| 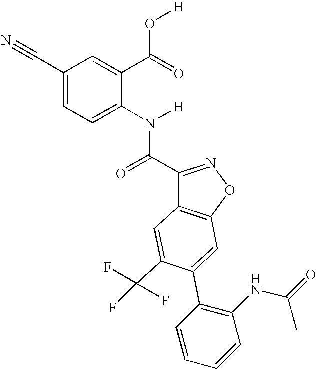 | 2-({[6-[2-(acetylamino)phenyl]-5-(trifluoromethyl)- 1,2-benzisoxazol-3-yl]carbonyl)amino)-5- cyanobenzoic acid |
| 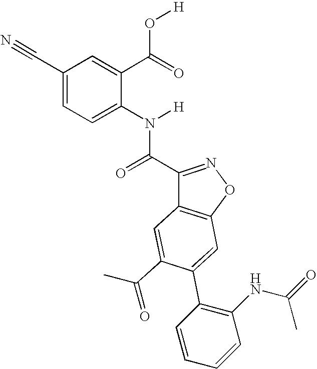 | 2-[({5-acetyl-6-[2-(acetylamino)phenyl]-1,2- benzisoxazol-3-yl}carbonyl)amino]-5- cyanobenzoic acid |
| 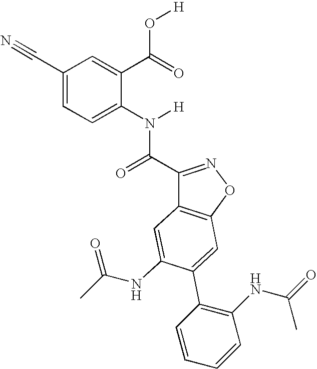 | 2-[({5-(acetylamino)-6-[2-(acetylamino)phenyl]- 1,2-benzisoxazol-3-yl}carbonyl)amino]- 5-cyanobenzoic acid |
| 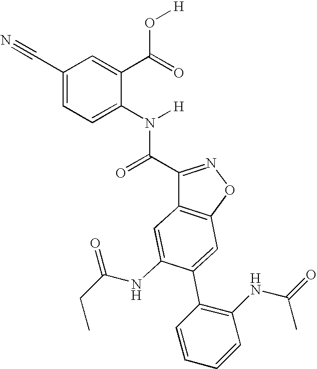 | 2-({[6-[2-(acetylamino)phenyl]-5-(propionylamino)-1,2- benzisoxazol-3-yl]carbonyl}amino)-5- cyanobenzoic acid |
| 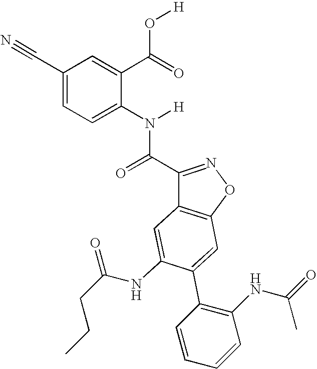 | 2-({[6-[2-(acetylamino)phenyl]-5-(butyrylamino)-1,2- benzisoxazol-3-yl]carbonyl}amino)-5- cyanobenzoic acid |
| 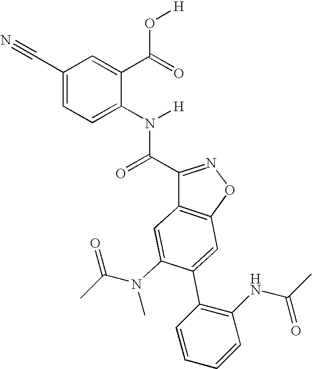 | 2-[({6-[2-(acetylamino)phenyl]-5-[acetyl(methyl) amino]-1,2-benzisoxazol-3-yl}carbonyl)amino]-5- cyanobenzoic acid |
| 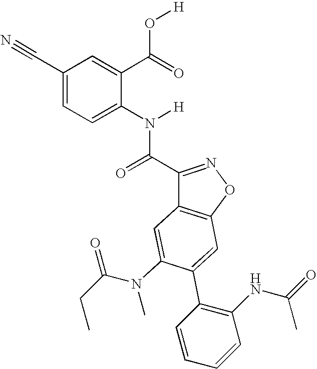 | 2-[({6-[2-(acetylamino)phenyl]-5-[methyl(propionyl) amino]-1,2-benzisoxazol-3-yl}carbonyl)amino]-5- cyanobenzoic acid |
| 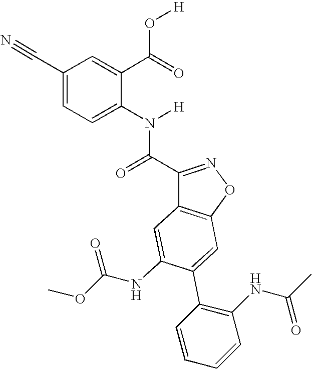 | 2-[({6-[2-(acetylamino)phenyl]-5-[(methoxycarbonyl) amino]-1,2-benzisoxazol-3-yl}carbonyl)amino]-5- cyanobenzoic acid |
| 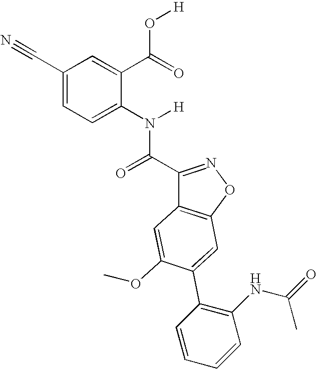 | 2-[({6-[2-(acetylamino)phenyl]-5-methoxy-1,2- benzisoxazol-3-yl}carbonyl)amino]-5- cyanobenzoic acid |
| 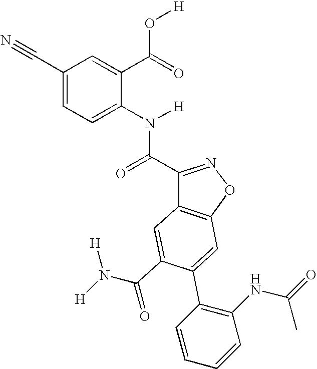 | 2-({[6-[2-(acetylamino)phenyl]-5-(aminocarbonyl)- 1,2-benzisoxazol-3-yl]carbonyl}amino)-5- cyanobenzoic acid |
| 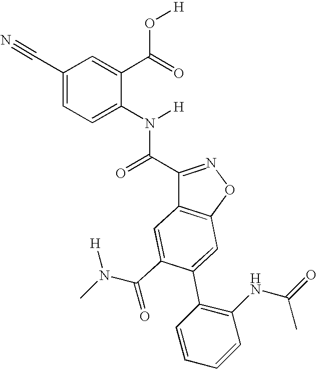 | 2-[({6-[2-(acetylamino)phenyl]-5-[(methylamino) carbonyl]-1,2-benzisoxazol-3-yl}carbonyl) amino]-5-cyanobenzoic acid |
| 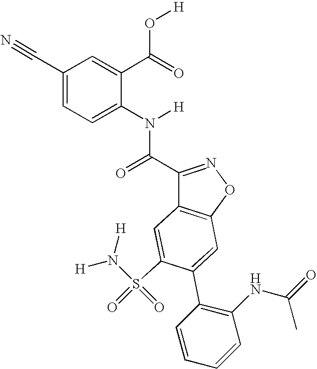 | 2-({[6-[2-(acetylamino)phenyl]-5-(aminosulfonyl)- 1,2-benzisoxazol-3-yl]carbonyl}amino)- 5-cyanobenzoic acid |
| 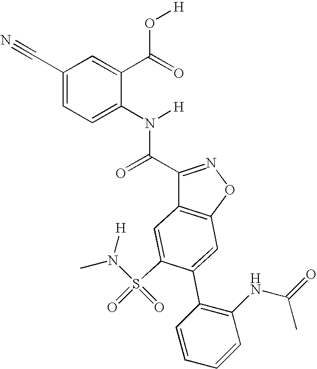 | 2-[({6-[2-(acetylamino)phenyl]-5-[(methylamino) sulfonyl]-1,2-benzisoxazol-3-yl}carbonyl)amino]-5- cyanobenzoic acid |
| 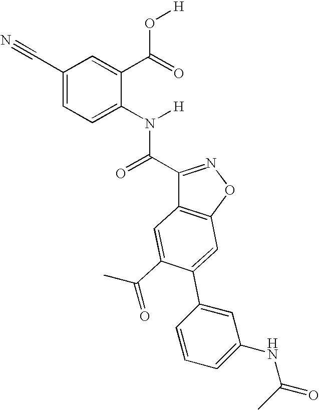 | 2-[({5-acetyl-6-[3-(acetylamino)phenyl]-1,2- benzisoxazol-3-yl}carbonyl)amino]-5- cyanobenzoic acid |
| 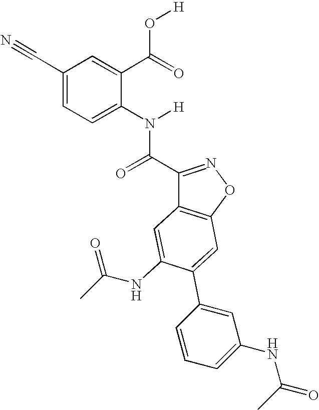 | 2-[({5-(acetylamino)-6-[3-(acetylamino)phenyl]- 1,2-benzisoxazol-3-yl}carbonyl)amino]- 5-cyanobenzoic acid |
| 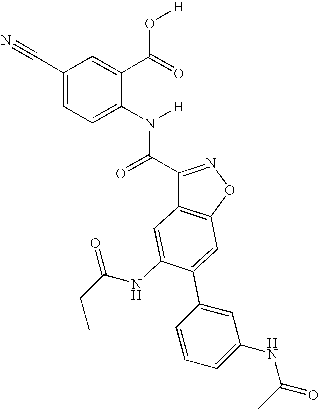 | 2-({[6-[3-(acetylamino)phenyl]-5- (propionylamino)-1,2-benzisoxazol-3-yl]carbonyl}amino)-5-cyanobenzoic acid |
| 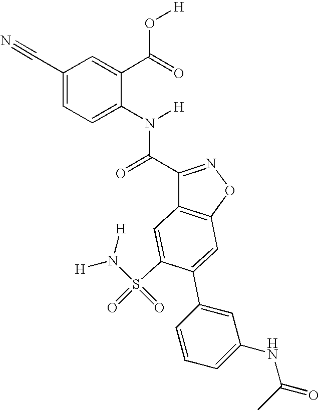 | 2-({[6-[3-(acetylamino)phenyl]-5- (aminosulfonyl)-1,2-benzisoxazol-3-yl]carbonyl}amino)-5-cyanobenzoic acid |
| 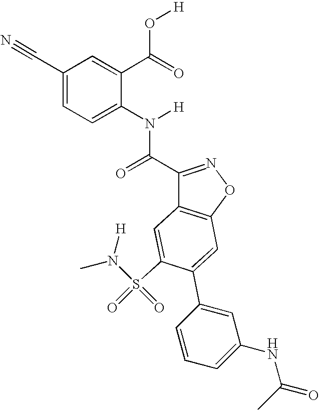 | 2-[({6-[3-(acetylamino)phenyl]-5- [(methylamino)sulfonyl]-1,2-benzisoxazol-3- yl}carbonyl)amino]-5-cyanobenzoic acid |
| 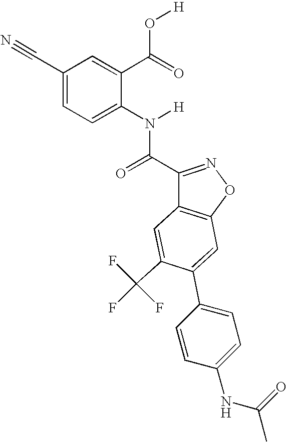 | 2-({[6-[4-(acetylamino)phenyl]-5- (trifluoromethyl)-1,2-benzisoxazol-3-yl]carbonyl}amino)-5-cyanobenzoic acid |
| 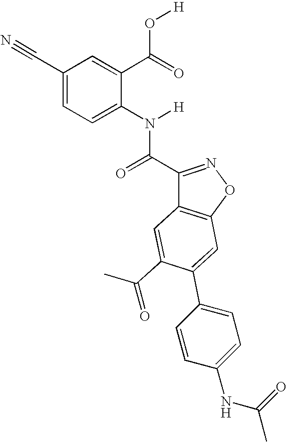 | 2-[({5-acetyl-6-[4-(acetylamino)phenyl]-1,2- benzisoxazol-3-yl}carbonyl)amino]-5- cyanobenzoic acid |
| 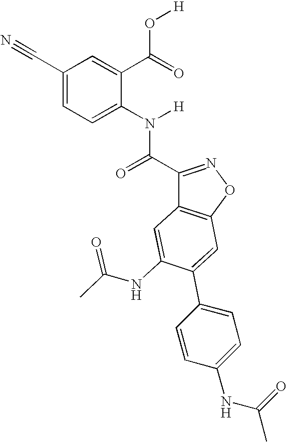 | 2-[({5-(acetylamino)-6-[4-(acetylamino) phenyl]-1,2-benzisoxazol-3-yl}carbonyl)amino]-5-cyanobenzoic acid |
| 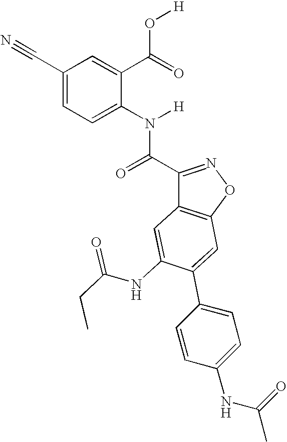 | 2-({[6-[4-(acetylamino)phenyl]-5- (propionylamino)-1,2-benzisoxazol-3-yl]carbonyl}amino)-5-cyanobenzoic acid |
| 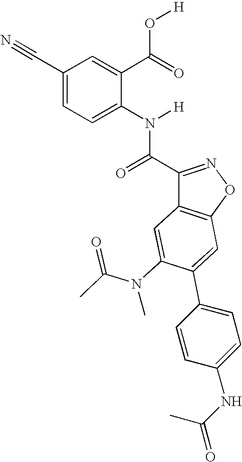 | 2-[({6-[4-(acetylamino)phenyl]-5-[acetyl (methyl)amino]-1,2-benzisoxazol-3-yl}carbonyl)amino]-5-cyanobenzoic acid |
| 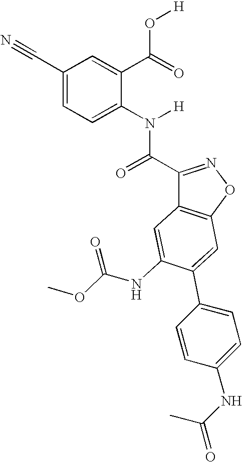 | 2-[({6-[4-(acetylamino)phenyl]-5- [(methoxycarbonyl)amino]-1,2-benzisoxazol-3- yl}carbonyl)amino]-5-cyanobenzoic acid |
| 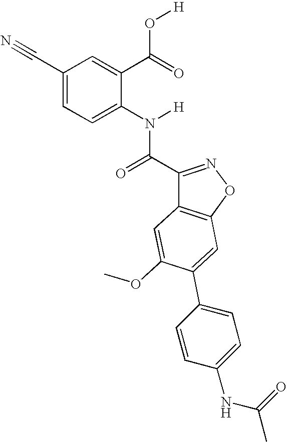 | 2-[({6-[4-(acetylamino)phenyl]-5-methoxy- 1,2-benzisoxazol-3-yl}carbonyl)amino]-5- cyanobenzoic acid |
| 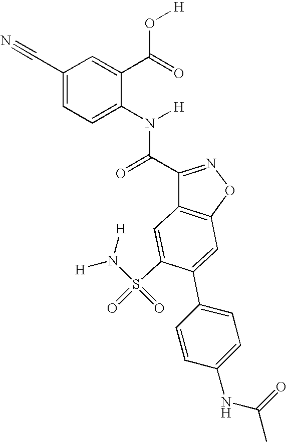 | 2-({[6-[4-(acetylamino)phenyl]-5- (aminosulfonyl)-1,2-benzisoxazol-3-yl]carbonyl}amino)-5-cyanobenzoic acid |
| 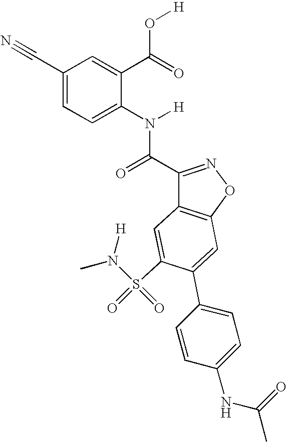 | 2-[({6-[4-(acetylamino)phenyl]-5- [(methylamino)sulfonyl]-1,2-benzisoxazol-3- yl}carbonyl)amino]-5-cyanobenzoic acid |
| 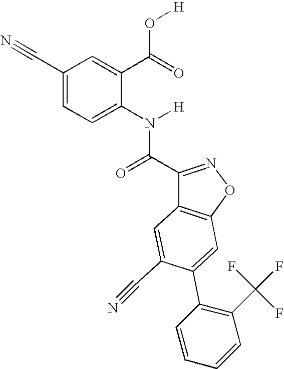 | 5-cyano-2-[({5-cyano-6-[2-(trifluoromethyl) phenyl]-1,2-benzisoxazol-3-yl}carbonyl) amino]benzoic acid |
| 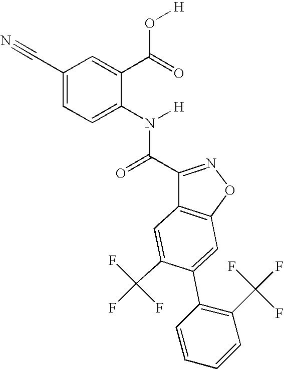 | 5-cyano-2-[({5-(trifluoromethyl)-6-[2- (trifluoromethyl)phenyl]-1,2-benzisoxazol-3- yl}carbonyl)amino]benzoic acid |
| 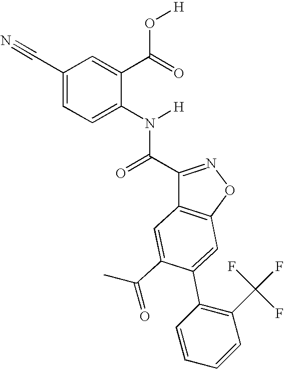 | 2-[({5-acetyl-6-[2-(trifluoromethyl)phenyl]-1,2- benzisoxazol-3-yl}carbonyl)amino]-5- cyanobenzoic acid |
| 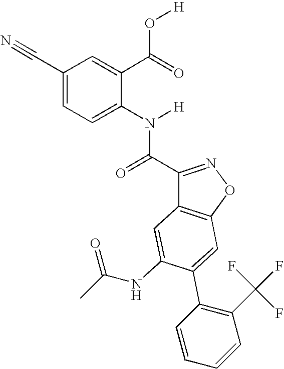 | 2-[({5-(acetylamino)-6-[2-(trifluoromethyl) phenyl]-1,2-benzisoxazol-3-yl}carbonyl) amino]-5-cyanobenzoic acid |
| 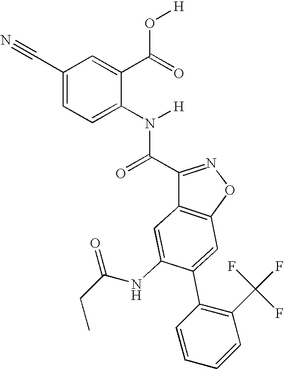 | 5-cyano-2-[({5-(propionylamino)-6-[2- (trifluoromethyl)phenyl]-1,2-benzisoxazol-3- yl}carbonyl)amino]benzoic acid |
| 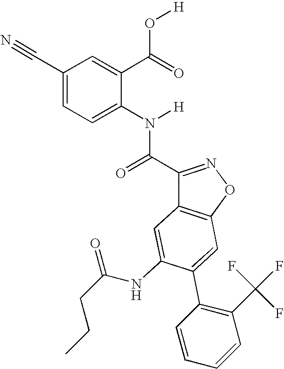 | 2-[({5-(butyrylamino)-6-[2-(trifluoromethyl) phenyl]-1,2-benzisoxazol-3-yl}carbonyl)amino]-5-cyanobenzoic acid |
| 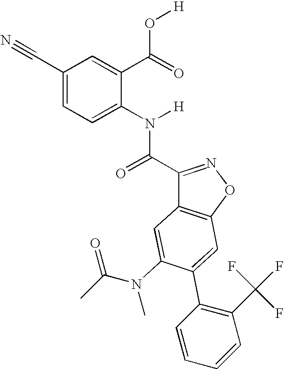 | 2-[({5-[acetyl(methyl)amino]-6-[2- (trifluoromethyl)phenyl]-1,2-benzisoxazol-3- yl}carbonyl)amino]-5-cyanobenzoic acid |
| 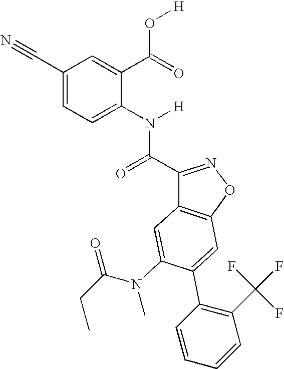 | 5-cyano-2-[({5-[methyl(propionyl)amino]-6-[2- (trifluoromethyl)phenyl]-1,2-benzisoxazol-3-yl}carbonyl)amino]benzoic acid |
| 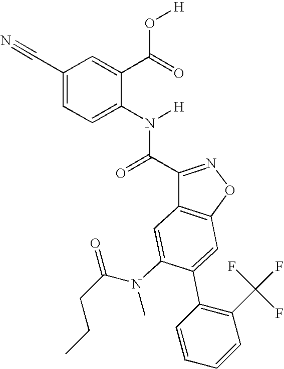 | 2-[({5-[butyryl(methyl)amino]-6-[2- (trifluoromethyl)phenyl]-1,2-benzisoxazol-3- yl}carbonyl)amino]-5-cyanobenzoic acid |
| 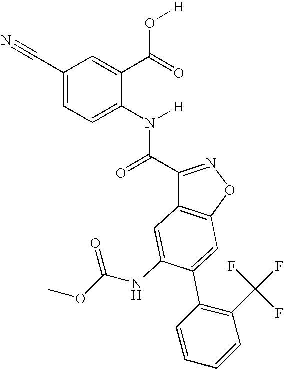 | 5-cyano-2-[({5-[(methoxycarbonyl)amino]-6-[2- (trifluoromethyl)phenyl]-1,2-benzisoxazol-3-yl}carbonyl)amino]benzoic acid |
| 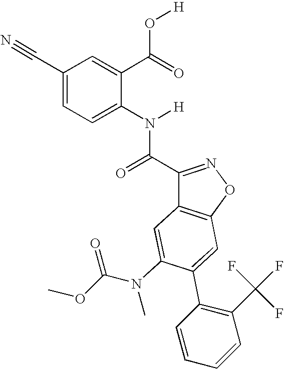 | 5-cyano-2-[({5-[(methoxycarbonyl)(methyl)amino]- 6-[2-(trifluoromethyl)phenyl]-1,2-benzisoxazol-3- yl}carbonyl)amino]benzoic acid |
| 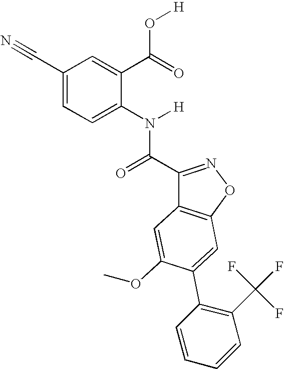 | 5-cyano-2-[({5-methoxy-6-[2-(trifluoromethyl)phenyl]-1,2- benzisoxazol-3-yl}carbonyl)amino]benzoic acid |
| 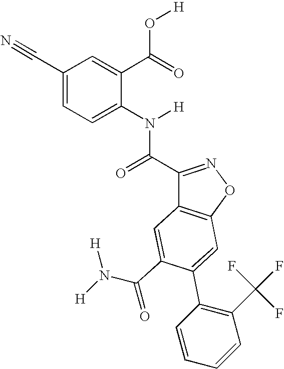 | 2-[({5-(aminocarbonyl)-6-[2-(trifluoromethyl)phenyl]-1,2- benzisoxazol-3-yl}carbonyl)amino]-5- cyanobenzoic acid |
| 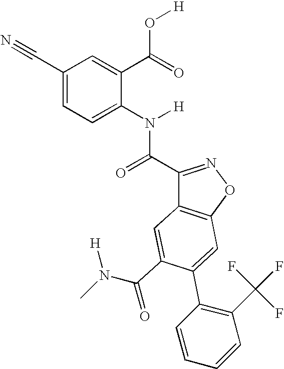 | 5-cyano-2-[({5-[(methylamino)carbonyl]-6-[2- (trifluoromethyl)phenyl]-1,2-benzisoxazol- 3-yl}carbonyl)amino]benzoic acid |
| 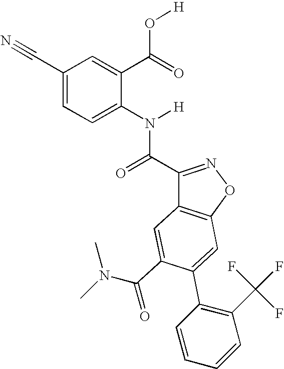 | 5-cyano-2-[({5-[(dimethylamino)carbonyl]-6-[2- (trifluoromethyl)phenyl]-1,2-benzisoxazol- 3-yl}carbonyl)amino]benzoic acid |
| 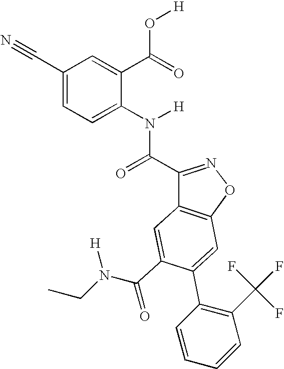 | 5-cyano-2-[({5-[(ethylamino)carbonyl]-6-[2- (trifluoromethyl)phenyl]-1,2-benzisoxazol-3- yl}carbonyl)amino]benzoic acid |
| 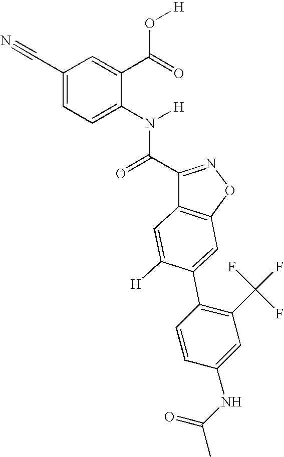 | 2-[({6-[4-(acetylamino)-2-(trifluoromethyl)phenyl]- 1,2-benzisoxazol-3-yl}carbonyl)amino]- 5-cyanobenzoic acid |
| 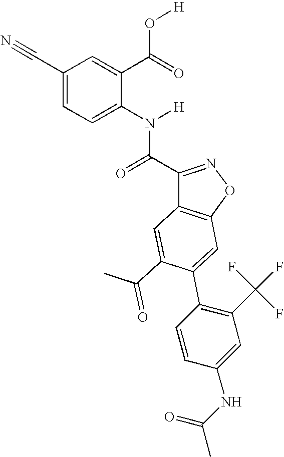 | 2-[({5-acetyl-6-[4-(acetylamino)-2- (trifluoromethyl)phenyl]-1,2-benzisoxazol-3- yl}carbonyl)amino]-5-cyanobenzoic acid |
| 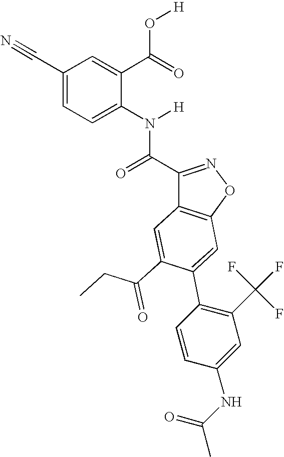 | 2-[({6-[4-(acetylamino)-2-(trifluoromethyl)phenyl]- 5-propionyl-1,2-benzisoxazol-3-yl}carbonyl)amino]- 5-cyanobenzoic acid |
| 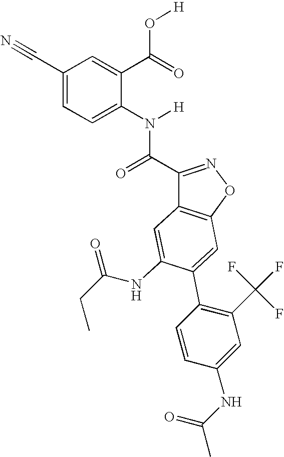 | 2-({[6-[4-(acetylamino)-2-(trifluoromethyl)phenyl]- 5-(propionylamino)-1,2-benzisoxazol-3-yl]carbonyl}amino)-5-cyanobenzoic acid |
| 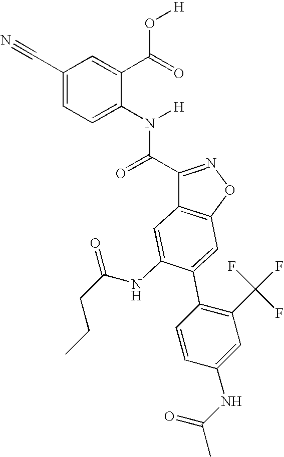 | 2-({[6-[4-(acetylamino)-2-(trifluoromethyl)phenyl]- 5-(butyrylamino)-1,2-benzisoxazol-3-yl]carbonyl}amino)-5-cyanobenzoic acid |
| 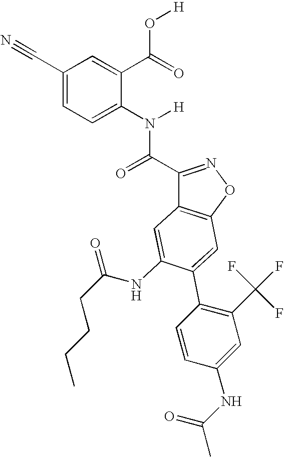 | 2-({[6-[4-(acetylamino)-2-(trifluoromethyl)phenyl]- 5-(pentanoylamino)-1,2-benzisoxazol-3-yl]carbonyl}amino)-5-cyanobenzoic acid |
| 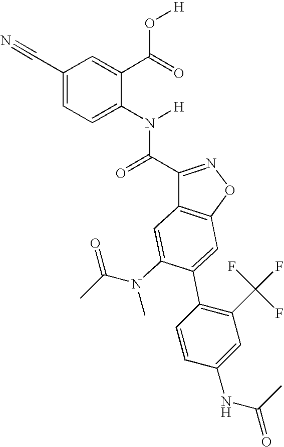 | 2-[({6-[4-(acetylamino)-2-(trifluoromethyl)phenyl]- 5-[acetyl(methyl)amino]-1,2-benzisoxazol-3-yl}carbonyl)amino]-5-cyanobenzoic acid |
| 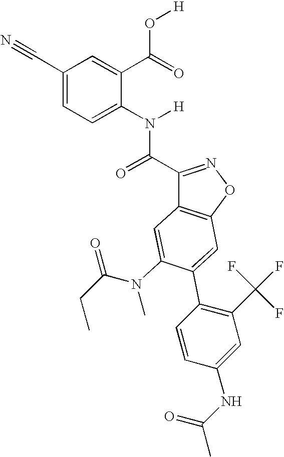 | 2-[({6-[4-(acetylamino)-2-(trifluoromethyl)phenyl]- 5-[methyl(propionyl)amino]-1,2-benzisoxazol-3-yl}carbonyl)amino]-5-cyanobenzoic acid |
| 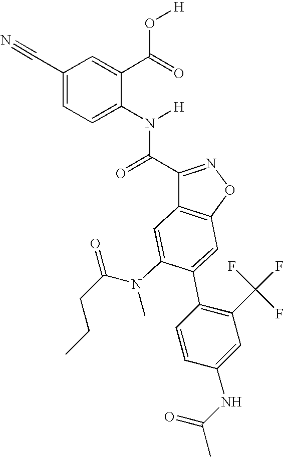 | 2-[({6-[4-(acetylamino)-2-(trifluoromethyl)phenyl]- 5-[butyryl(methyl)amino]-1,2-benzisoxazol-3-yl}carbonyl)amino]-5-cyanobenzoic acid |
| 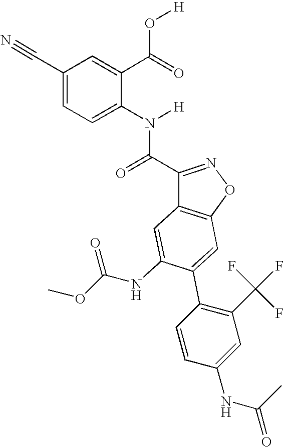 | 2-[({6-[4-(acetylamino)-2-(trifluoromethyl)phenyl]- 5-[(methoxycarbonyl)amino]-1,2-benzisoxazol-3-yl}carbonyl)amino]-5-cyanobenzoic acid |
| 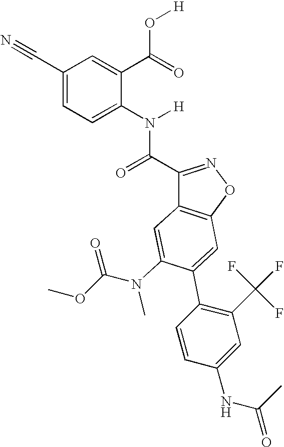 | 2-[({6-[4-(acetylamino)-2-(trifluoromethyl)phenyl]- 5-[(methoxycarbonyl)(methyl)amino]-1,2-benzisoxazol- 3-yl}carbonyl)amino]-5-cyanobenzoic acid |
| 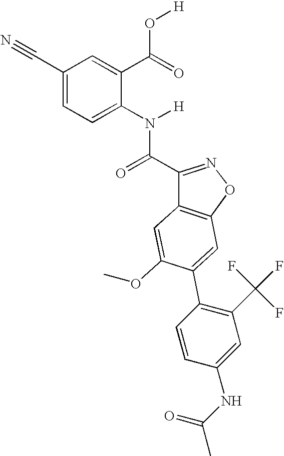 | 2-[({6-[4-(acetylamino)-2-(trifluoromethyl)phenyl]- 5-methoxy-1,2-benzisoxazol-3-yl}carbonyl)amino]-5- cyanobenzoic acid |
| 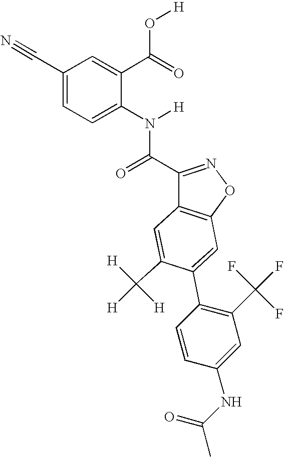 | 2-[({6-[4-(acetylamino)-2-(trifluoromethyl)phenyl]- 5-methyl-1,2-benzisoxazol-3-yl}carbonyl)amino]-5- cyanobenzoic acid |
| 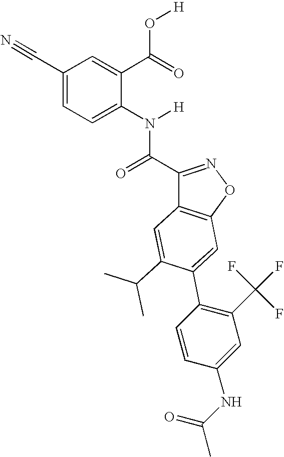 | 2-[({6-[4-(acetylamino)-2-(trifluoromethyl)phenyl]- 5-isopropyl-1,2-benzisoxazol-3-yl}carbonyl)amino]- 5-cyanobenzoic acid |
| 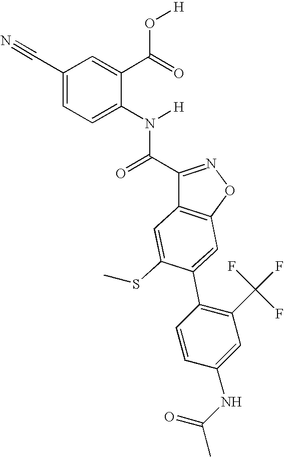 | 2-({[6-[4-(acetylamino)-2-(trifluoromethyl)phenyl]- 5-(methylthio)-1,2-benzisoxazol-3-yl]carbonyl}amino)- 5-cyanobenzoic acid |
| 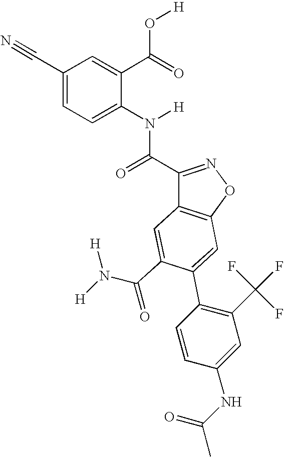 | 2-({[6-[4-(acetylamino)-2-(trifluoromethyl)phenyl]- 5-(aminocarbonyl)-1,2-benzisoxazol-3-yl]carbonyl}amino)- 5-cyanobenzoic acid |
| 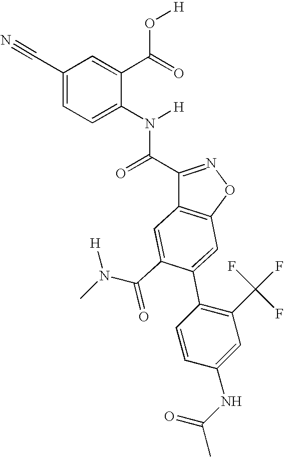 | 2-[({6-[4-(acetylamino)-2-(trifluoromethyl)phenyl]- 5-[(methylamino)carbonyl]-1,2-benzisoxazol-3-yl}carbonyl)amino]-5-cyanobenzoic acid |
| 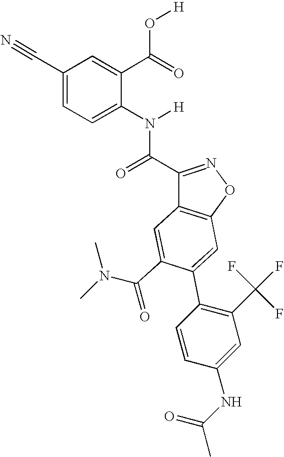 | 2-[({6-[4-(acetylamino)-2-(trifluoromethyl)phenyl]- 5-[(dimethylamino)carbonyl]-1,2-benzisoxazol-3- yl}carbonyl)amino]-5-cyanobenzoic acid |
| 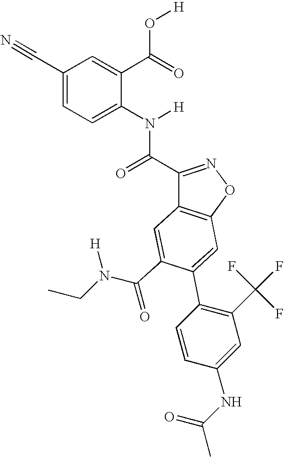 | 2-[({6-[4-(acetylamino)-2-(trifluoromethyl)phenyl]- 5-[(ethylamino)carbonyl]-1,2-benzisoxazol-3- yl}carbonyl)amino]-5-cyanobenzoic acid |
| 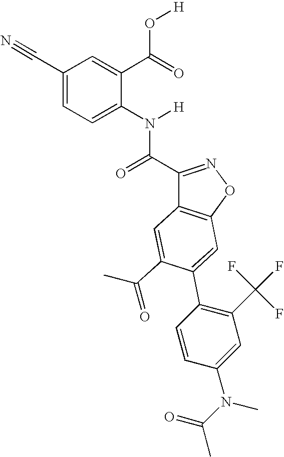 | 2-[({5-acetyl-6-[4-[acetyl(methyl)amino]-2- (trifluoromethyl)phenyl]-1,2-benzisoxazol-3- yl}carbonyl)amino]-5-cyanobenzoic acid |
| 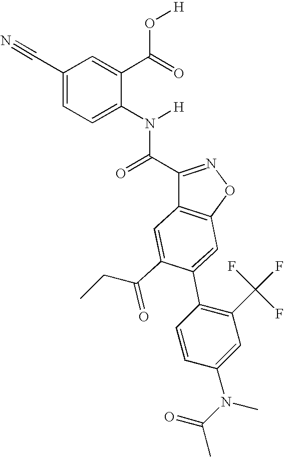 | 2-[({6-[4-[acetyl(methyl)amino]-2-(trifluoromethyl) phenyl]-5-propionyl-1,2-benzisoxazol-3-yl}carbonyl)amino]-5-cyanobenzoic acid |
| 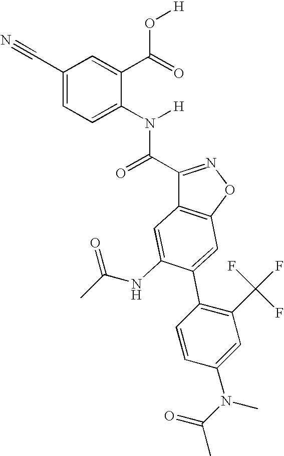 | 2-[({5-(acetylamino)-6-[4-[acetyl(methyl)amino]-2- (trifluoromethyl)phenyl]-1,2-benzisoxazol-3- yl}carbonyl)amino]-5-cyanobenzoic acid |
| 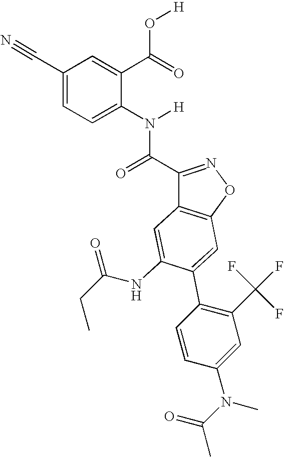 | 2-({[6-[4-[acetyl(methyl)amino]-2-(trifluoromethyl)phenyl]- 5-(proptonylamino)-1,2-benzisoxazol-3-yl]carbonyl}amino)-5-cyanobenzoic acid |
| 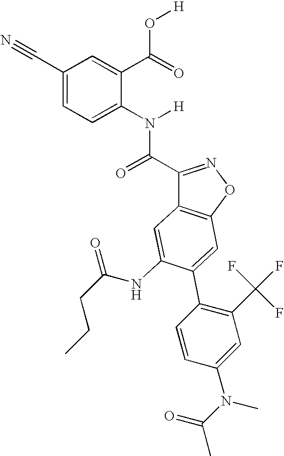 | 2-({[6-[4-]acetyl(methyl)amino]-2-(trifluoromethyl)phenyl]- 5-(butyrylamino)-1,2-benzisoxazol-3-yl]carbonyl}amino)-5- cyanobenzoic acid |
| 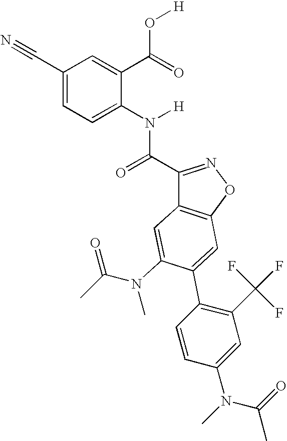 | 2-[({5-[acetyl(methyl)amino]-6-[4-[acetyl(methyl)amino]- 2-(trifluoromethyl)phenyl]-1,2-benzisoxazol-3- yl}carbonyl)amino]-5-cyanobenzoic acid |
| 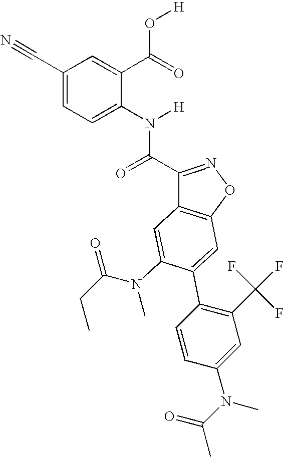 | 2-[({6-[4-[acetyl(methyl)amino]-2-(trifluoromethyl) phenyl]-5-[methyl(propionyl)amino]-1,2-benzisoxazol- 3-yl]carbonyl)amino]-5-cyanobenzoic acid |
| 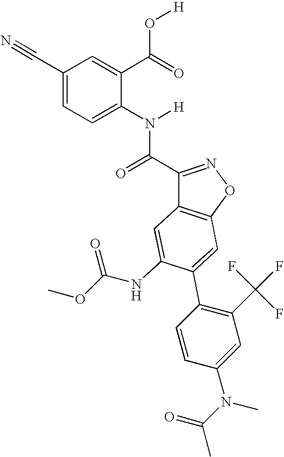 | 2-[({6-[4-[acetyl(methyl)amino]-2-(trifluoromethyl) phenyl]-5-[(methoxycarbonyl)amino]-1,2-benzisoxazol- 3-yl]carbonyl)amino]-5-cyanobenzoic acid |
| 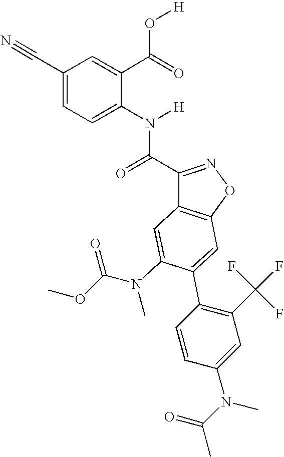 | 2-[({6-[4-[acetyl(methyl)amino]-2-(trifluoromethyl) phenyl]-5-[(methoxycarbonyl)(methyl)amino]-1,2- benzisoxazol-3-yl}carbonyl)amino]-5- cyanobenzoic acid |
| 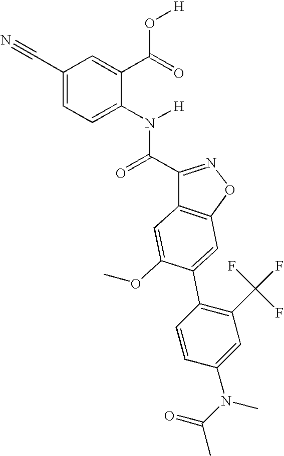 | 2-[({6-[4-[acetyl(methyl)amino]-2-(trifluoromethyl) phenyl]-5-methoxy-1,2-benzisoxazol-3-yl}carbonyl)amino]- 5-cyanobenzoic acid |
| 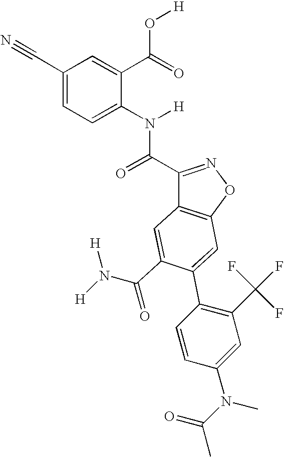 | 2-({[6-[4-[acetyl(methyl)amino]-2-(trifluoromethyl) phenyl]-5-(aminocarbonyl)-1,2-benzisoxazol-3-yl]carbonyl) amino)-5-cyanobenzoic acid |
| 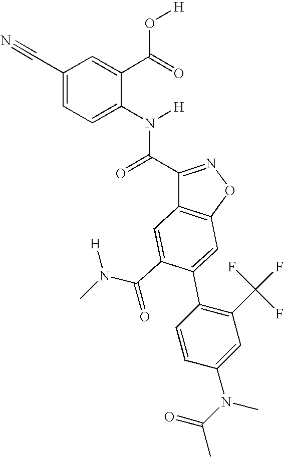 | 2-[({6-[4-[acetyl(methyl)amino]-2-(trifluoromethyl) phenyl]-5-[(methylamino)carbonyl]-1,2- benzisoxazol-3-yl}carbonyl)amino]- 5-cyanobenzoic acid |
| 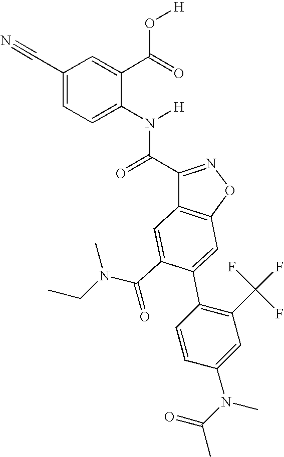 | 2-{[(6-[4-[acetyl(methyl)amino]-2-(trifluoromethyl) phenyl]-5-{[ethyl(methyl)amino]carbonyl}-1,2- benzisoxazol-3-yl)carbonyl]amino}-5- cyanobenzoic acid |
| 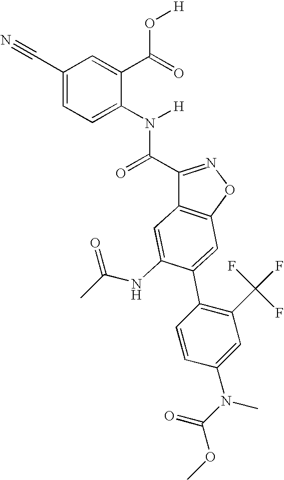 | 2-[({5-(acetylamino)-6-[4-[(methoxycarbonyl)(methyl) amino]-2-(trifluoromethyl)phenyl]-1,2-benzisoxazol-3-yl}carbonyl)amino]-5-cyanobenzoic acid |
| 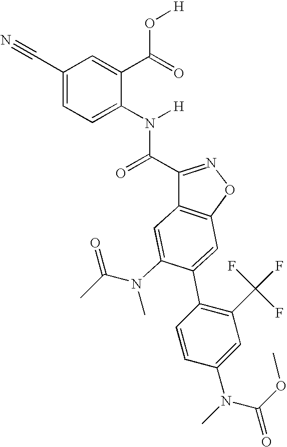 | 2-[({5-[acetyl(methyl)amino]-6-[4-[(methoxycarbonyl) (methyl)amino]-2-(trifluoromethyl)phenyl]-1,2- benzisoxazol-3-yl}carbonyl)amino]-5- cyanobenzoic acid |
| 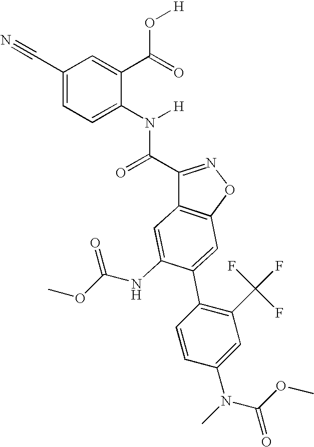 | 5-cyano-2-[({5-](methoxycarbonyl)amino]-6-[4- [(methoxycarbonyl)(methyl)amino]-2- (trifluoromethyl)phenyl]-1,2-benzisoxazol-3- yl}carbonyl)amino]benzoic acid |
| 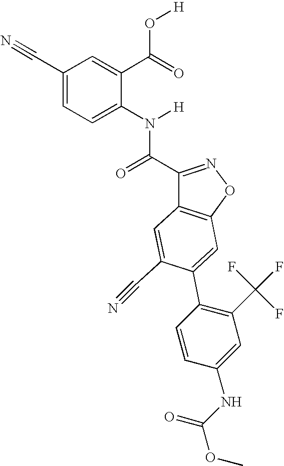 | 5-cyano-2-[({5-cyano-6-[4-[(methoxycarbonyl)amino]- 2-(trifluoromethyl)phenyl]-1,2-benzisoxazol-3- yl}carbonyl)amino]benzoic acid |
| 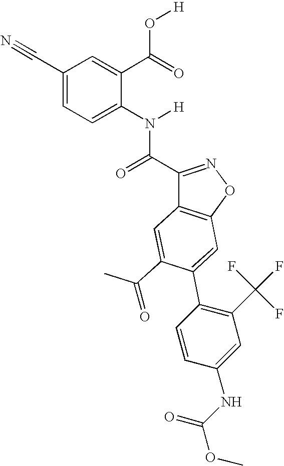 | 2-[({5-acetyl-6-[4-[(methoxycarbonyl)amino]-2- (trifluoromethyl)phenyl]-1,2-benzisoxazol-3- yl]carbonyl)amino]-5-cyanobenzoic acid |
| 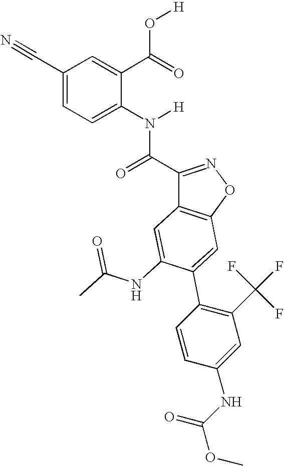 | 2-[({5-(acetylamino)-6-[4-[(methoxycarbonyl)amino]- 2-(trifluoromethyl)phenyl]-1,2-benzisoxazol-3- yl}carbonyl)amino]-5-cyanobenzoic acid |
| 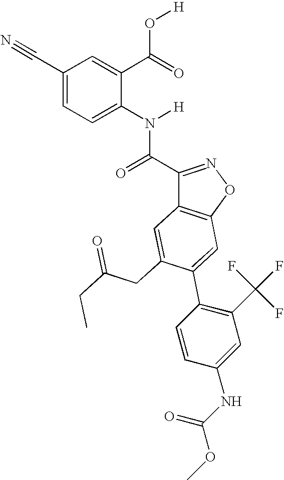 | 5-cyano-2-({[6-[4-[(methoxycarbonyl)amino]-2- (trifluoromethyl)phenyl]-5-(propionylamino)-1,2- benzisoxazol-3-yl]carbonyl]amino)benzoic acid |
| 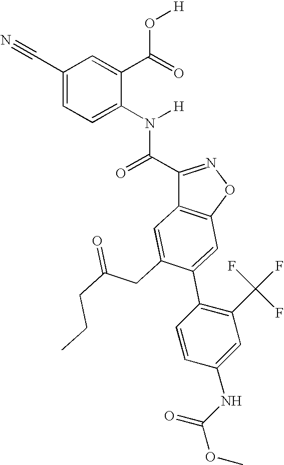 | 2-[({5-(butyrylamino)-5-[4-[(methoxycarbonyl)amino]- 2-(trifluoromethyl)phenyl]-1,2-benzisoxazol-3- yl}carbonyl)amino]-5-cyanobenzoic acid |
| 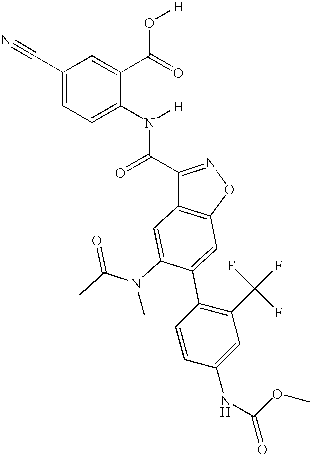 | 2-[({5-[acetyl(methyl)amino]-6-[4-[(methoxycarbonyl) amino]-2-(trifluoromethyl)phenyl]-1,2-benzisoxazol-3- yl}carbonyl)amino]-5-cyanobenzoic acid |
| 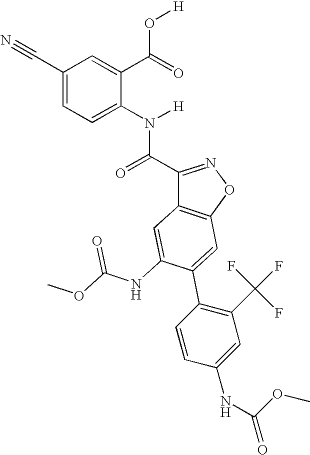 | 5-cyano-2-[({5-[(methoxycarbonyl)amino]-6- [4-[(methoxycarbonyl)amino]-2- (trifluoromethyl)phenyl]-1,2-benzisoxazol-3- yl}carbonyl)amino]benzoic acid |
| 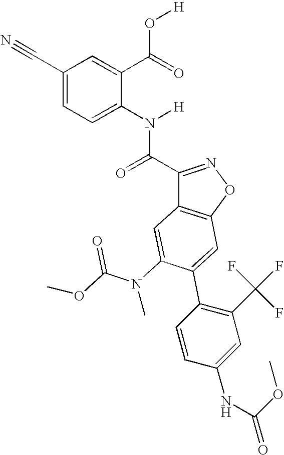 | 5-cyano-2-[({6-[4-[(methoxycarbonyl)amino]- 2-(trifluoromethyl)phenyl]-5- [(methoxycarbonyl)(methyl)amino]-1,2- benzisoxazol-3-yl}carbonyl)amino]benzoic acid |
| 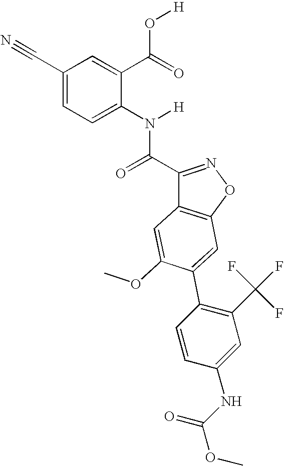 | 5-cyano-2-[({5-methoxy-6-[4-[(methoxycarbonyl) amino]-2-(trifluoromethyl)phenyl]-1,2- benzisoxazol-3-yl}carbonyl)amino]benzoic acid |
| 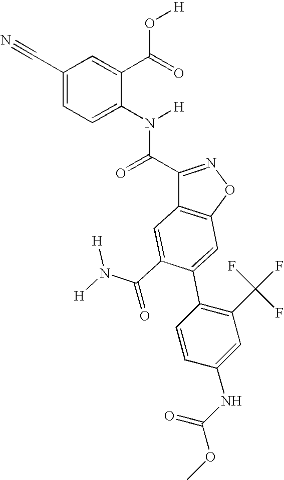 | 2-[({5-(aminocarbonyl)-6-[4-[(methoxycarbonyl) amino]-2-(trifluoromethyl)phenyl]-1,2- benzisoxazol-3-yl}carbonyl)amino]-5- cyanobenzoic acid |
| 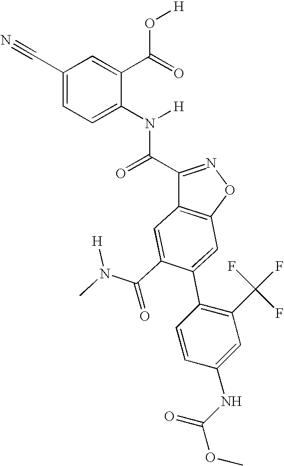 | 5-cyano-2-[({6-[4-[(methoxycarbonyl)amino]- 2-(trifluoromethyl)phenyl]-5- [(methylamino)carbonyl]-1,2-benzisoxazol-3- yl}carbonyl)amino]benzoic acid |
| 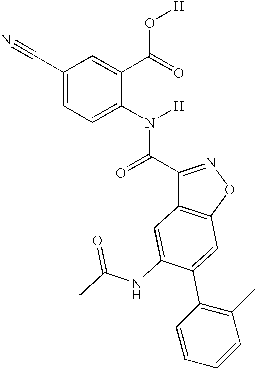 | 2-({[5-(acetylamino)-6-(2-methylphenyl)-1,2- benzisoxazol-3-yl]carbonyl}amino)-5- cyanobenzoic acid |
| 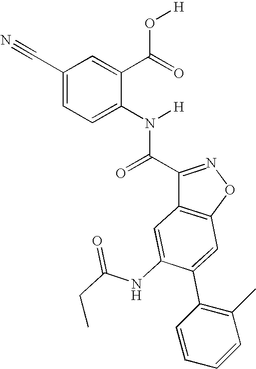 | 5-cyano-2-({[6-(2-methylphenyl)-5- (propionylamino)-1,2-benzisoxazol-3- yl]carbonyl}amino)benzoic acid |
| 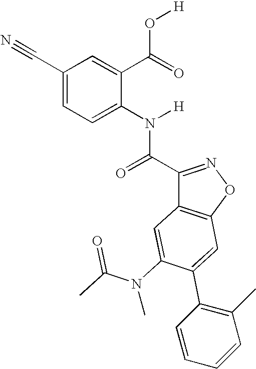 | 2-({[5-[acetyl(methyl)amino]-6-(2- methylphenyl)-1,2-benzisoxazol-3- yl]carbonyl}amino)-5-cyanobenzoic acid |
| 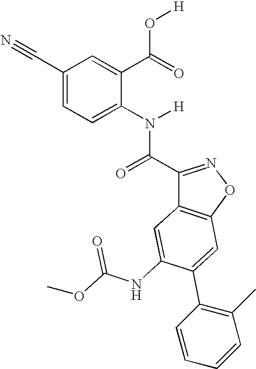 | 5-cyano-2-({[5-[(methoxycarbonyl)amino]-6- (2-methylphenyl)-1,2-benzisoxazol-3- yl]carbonyl}amino)benzoic acid |
| 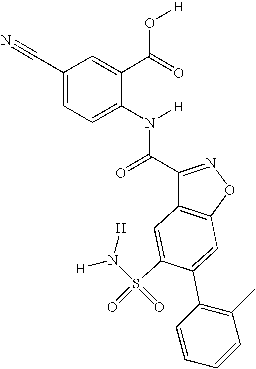 | 2-({[5-(aminosulfonyl)-6-(2-methylphenyl)- 1,2-benzisoxazol-3-yl]carbonyl}amino)-5- cyanobenzoic acid |
| 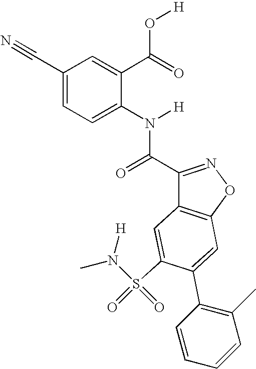 | 5-cyano-2-({[5-[(methylamino)sulfonyl]-6-(2- methylphenyl)-1,2-benzisoxazol-3-yl]carbonyl}amino)benzoic acid |
| 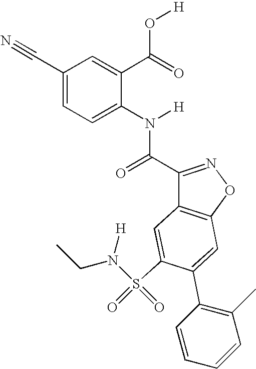 | 5-cyano-2-({[5-[(ethylamino)sulfonyl]-6-(2- methylphenyl)-1,2-benzisoxazol-3-yl]carbonyl}amino)benzoic acid |
| 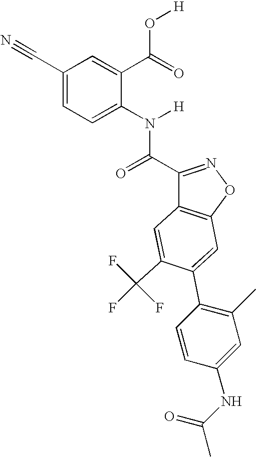 | 2-({[6-[4-(acetylamino)-2-methylphenyl]-5- (trifluoromethyl)-1,2-benzisoxazol-3-yl]carbonyl}amino)-5-cyanobenzoic acid |
| 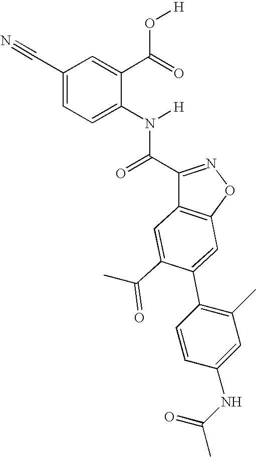 | 2-[({5-acetyl-6-[4-(acetylamino)-2-methylphenyl]-1,2- benzisoxazol-3-yl}carbonyl)amino]-5-cyanobenzoic acid |
| 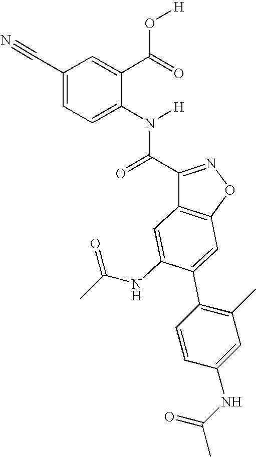 | 2-[({5-(acetylamino)-6-[4-(acetylamino)-2- methylphenyl]-1,2-benzisoxazol-3-yl}carbonyl)amino]- 5-cyanobenzoic acid |
| 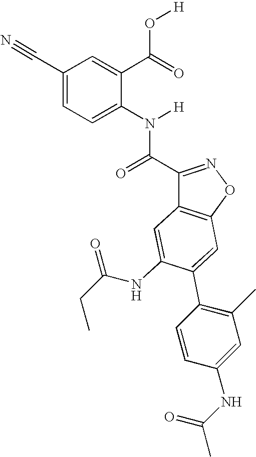 | 2-({[6-[4-(acetylamino)-2-methylphenyl]-5- (propionylamino)-1,2-benzisoxazol-3- yl]carbonyl}amino)-5-cyanobenzoic acid |
| 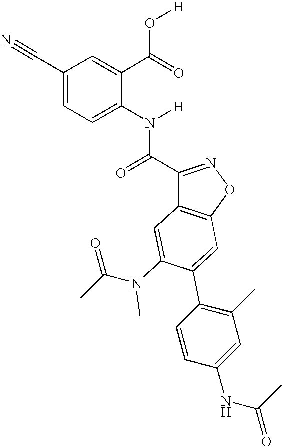 | 2-[({6-[4-(acetylamino)-2-methylphenyl]-5- [acetyl(methyl)amino]-1,2-benzisoxazol-3- yl}carbonyl)amino]-5-cyanobenzoic acid |
| 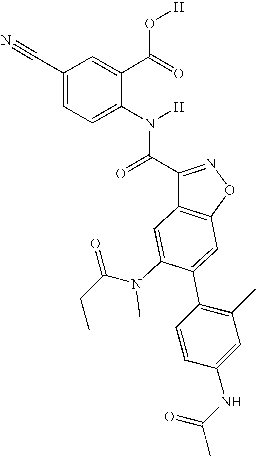 | 2-[({6-[4-(acetylamino)-2-methylphenyl]-5- [methyl(propionyl)amino]-1,2-benzisoxazol- 3-yl}carbonyl)amino]-5-cyanobenzoic acid |
| 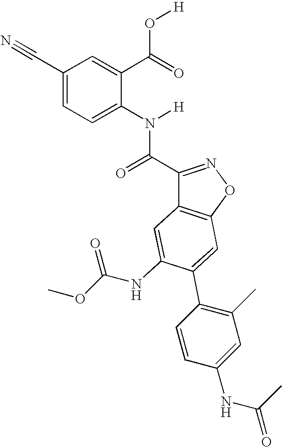 | 2-[({6-[4-(acetylamino)-2-methylphenyl]-5- [(methoxycarbonyl)amino]-1,2-benzisoxazol- 3-yl}carbonyl)amino]-5-cyanobenzoic acid |
| 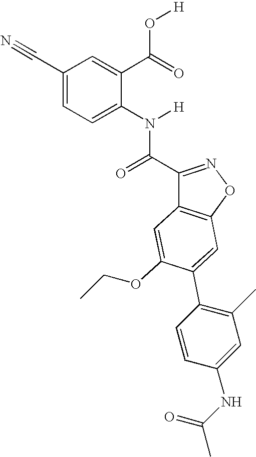 | 2-[({6-[4-(acetylamino)-2-methylphenyl]-5- ethoxy-1,2-benzisoxazol-3-yl]carbonyl)amino]-5- cyanobenzoic acid |
| 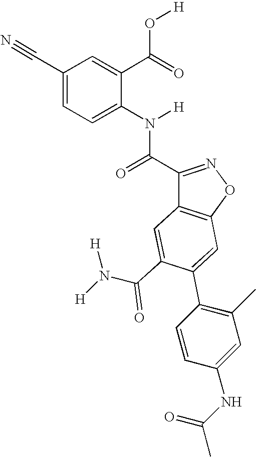 | 2-({[6-[4-(acetylamino)-2-methylphenyl]-5- (aminocarbonyl)-1,2-benzisoxazol-3- yl]carbonyl}amino)-5-cyanobenzoic acid |
| 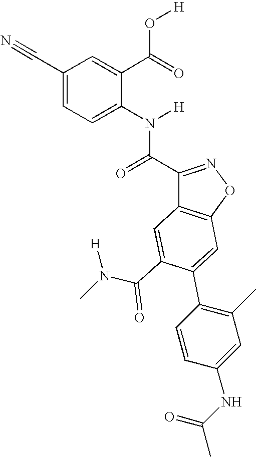 | 2-[({6-[4-(acetylamino)-2-methylphenyl]-5- [(methylamino)carbonyl]-1,2-benzisoxazol-3- yl]carbonyl)amino]-5-cyanobenzoic acid |
| 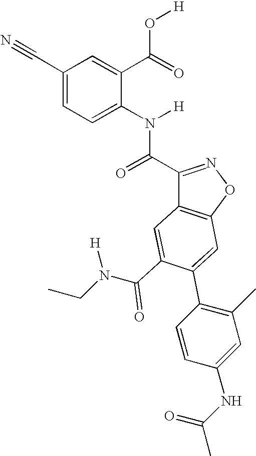 | 2-[({6-[4-(acetylamino)-2-methylphenyl]-5- [(ethylamino)carbonyl]-1,2-benzisoxazol-3- yl}carbonyl)amino]-5-cyanobenzoic acid |
| 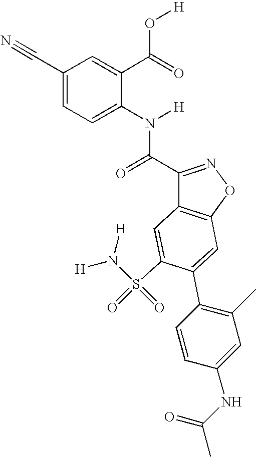 | 2-({[6-[4-(acetylamino)-2-methylphenyl]-5- (aminosulfonyl)-1,2-benzisoxazol-3- yl]carbonyl}amino)-5-cyanobenzoic acid |
| 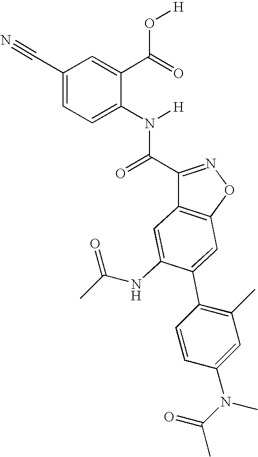 | 2-{[(5-(acetylamino)-6-{4-[acetyl(methyl)amino]-2- methylphenyl}-1,2-benzisoxazol-3-yl)carbonyl]amino}-5-cyanobenzoic acid |
| 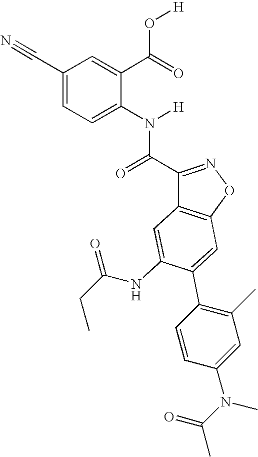 | 2-({[6-{4-[acetyl(methyl)amino]-2- methylphenyl}-5-(propionylamino)-1,2- benzisoxazol-3-yl]carbonyl}amino)-5- cyanobenzoic acid |
| 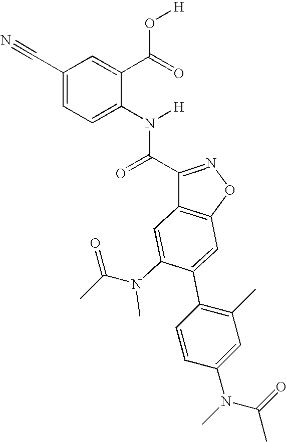 | 2-{[(5-[acetyl(methyl)amino]-6-{5- [acetyl(methyl)amino]-2-methylphenyl}-1,2- benzisoxazol-3-yl)carbonyl]amino}-5- cyanobenzoic acid |
| 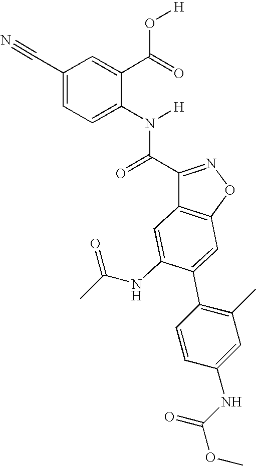 | 2-{[(5-(acetylamino)-6-{4-[(methoxycarbonyl)amino]-2- methylphenyl}-1,2-benzisoxazol-3-yl)carbonyl]amino}- 5-cyanobenzoic acid |
| 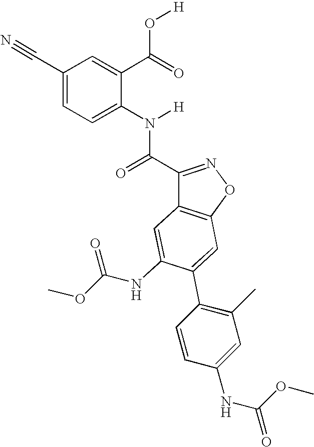 | 5-cyano-2-{[(5-[(methoxycarbonyl)amino]-6-{4- [(methoxycarbonyl)amino]-2-methylphenyl}-1,2- benzisoxazol-3-yl)carbonyl]amino}benzoic acid |
| 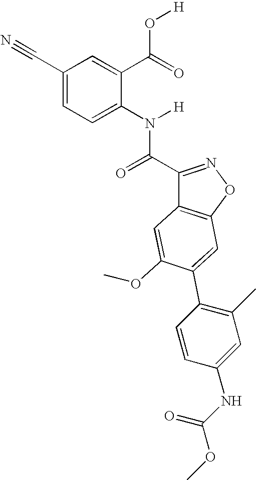 | 5-cyano-2-{[(5-methoxy-6-{4-[(methoxycarbonyl)amino]- 2-methylphenyl}-1,2-benzisoxazol-3-yl) carbonyl]amino}benzoic acid |
| 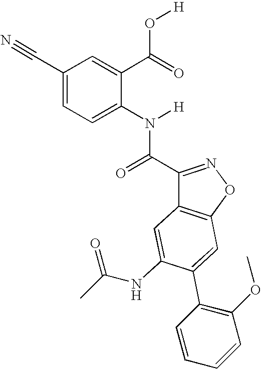 | 2-({[5-(acetylamino)-6-(2-methoxyphenyl)-1,2- benzisoxazol-3-yl]carbonyl}amino)-5- cyanobenzoic acid |
| 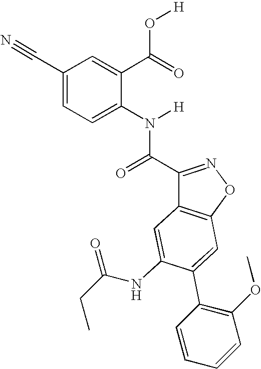 | 5-cyano-2-({[6-(2-methoxyphenyl)-5-(propionylamino)-1,2- benzisoxazol-3-yl]carbonyl}amino) benzoic acid |
| 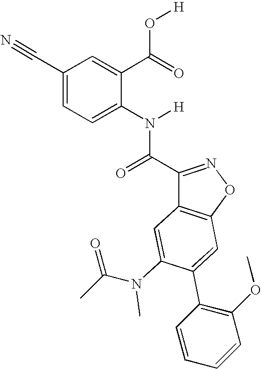 | 2-({[5-[acetyl(methyl)amino]-6-(2-methoxyphenyl)- 1,2-benzisoxazol-3-yl]carbonyl}amino)-5-cyanobenzoic acid |
| 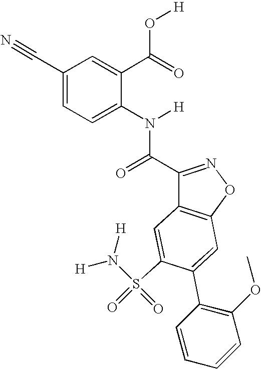 | 2-({[5-(aminosulfonyl)-6-(2-methoxyphenyl)-1,2- benzisoxazol-3-yl]carbonyl}amino)- 5-cyanobenzoic acid |
| 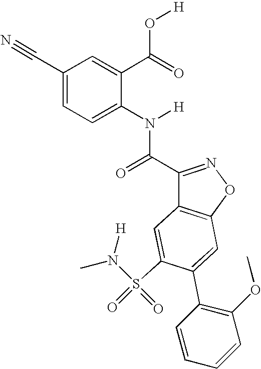 | 5-cyano-2-[({6-(2-methoxyphenyl)-5-[(methylamino) sulfonyl]-1,2-benzisoxazol-3-yl}carbonyl)amino]benzoic acid |
| 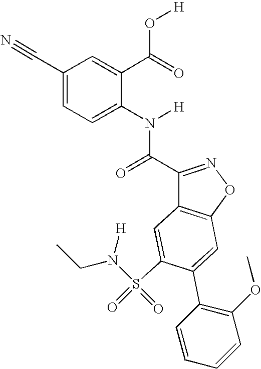 | 5-cyano-2-({[5-[(ethylamino)sulfonyl]-6-(2- methoxyphenyl)-1,2-benzisoxazol-3-yl]carbonyl}amino)benzoic acid |
| 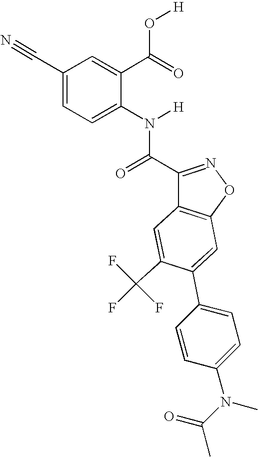 | 2-({[6-{4-[acetyl(methyl)amino]phenyl}-5- (trifluoromethyl)-1,2-benzisoxazol-3- yl]carbonyl}amino)-5-cyanobenzoic acid |
| 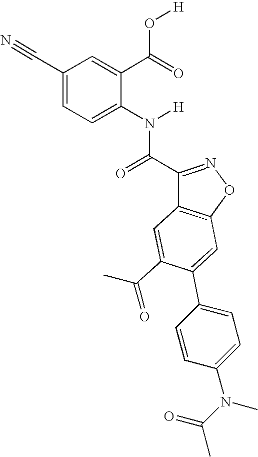 | 2-{[(5-acetyl-6-{4-[acetyl(methyl)amino]phenyl}-1,2- benzisoxazol-3-yl)carbonyl]amino}-5- cyanobenzoic acid |
| 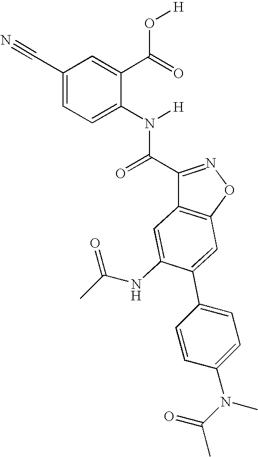 | 2-{[(5-(acetylamino)-6-{-[acetyl(methyl)amino]phenyl}-1,2-benzisoxazol-3-yl)carbonyl]amino}-5- cyanobenzoic acid |
| 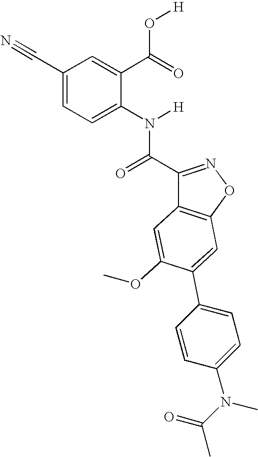 | 2-{([6-{4-[acetyl(methyl)amino]phenyl}-5- methoxy-1,2-benzisoxazol-3-yl)carbonyl]amino}-5- cyanobenzoic acid |
| 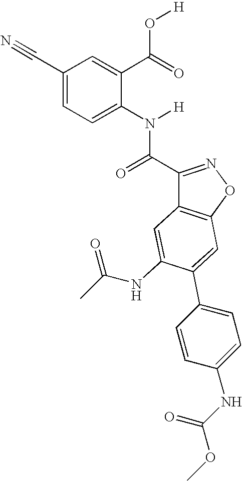 | 2-{[(5-(acetylamino)-6-{4-[(methoxycarbonyl)amino]phenyl}-1,2-benzisoxazol-3-yl)carbonyl]amino}-5- cyanobenzoic acid |
| 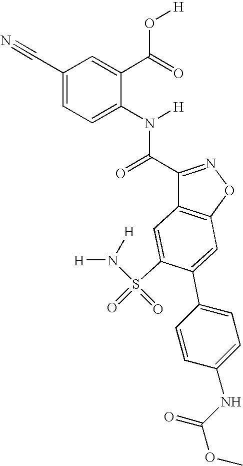 | 2-{[(5-(aminosulfonyl)-6-{4-[(methoxycarbonyl)amino]phenyl}-1,2-benzisoxazol-3-yl)carbonyl]amino}- 5-cyanobenzoic acid |
| 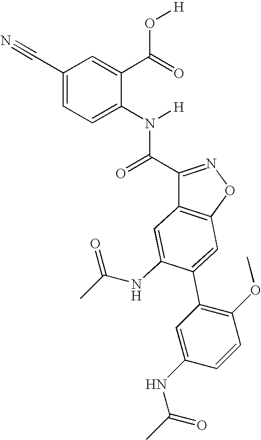 | 2-[({5-(acetylamino)-6-[5-(acetylamino)-2- methoxyphenyl]-1,2-benzisoxazol-3-yl]carbonyl) amino]-5-cyanobenzoic acid |
| 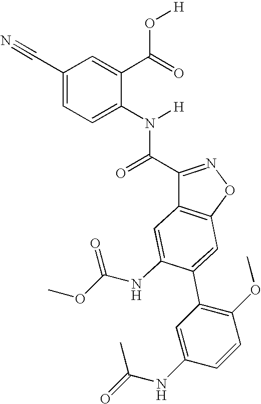 | 2-[({6-[5-(acetylamino)-2-methoxyphenyl]-5- [(methoxycarbonyl)amino]-1,2-benzisoxazol- 3-yl}carbonyl)amino]-5-cyanobenzoic acid |
| 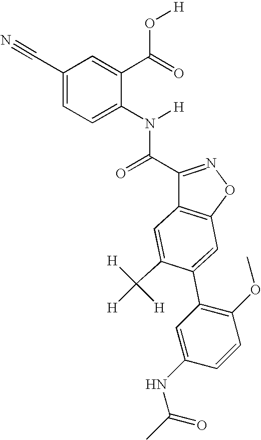 | 2-[({6-[5-(acetylamino)-2-methoxyphenyl]-5- methyl-1,2-benzisoxazol-3-yl}carbonyl)amino]-5- cyanobenzoic acid |
| 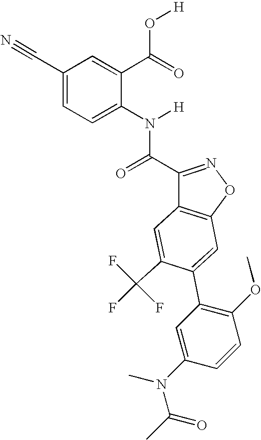 | 2-({[6-{5-[acetyl(methyl)amino]-2-methoxyphenyl}-5- (trifluoromethyl)-1,2-benzisoxazol-3-yl]carbonyl}amino)-5-cyanobenzoic acid |
| 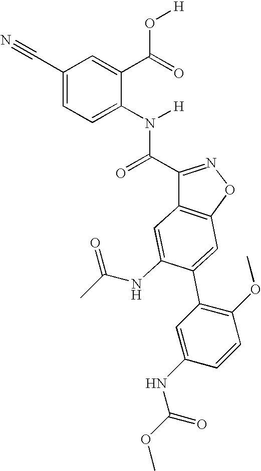 | 2-{[(5-(acetylamino)-6-{2-methoxy-5-[(methoxycarbonyl) amino]phenyl}-1,2-benzisoxazoI-3-yl)carbonyl]amino}-5-cyanobenzoic acid |
| 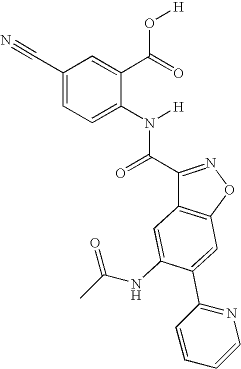 | 2-({[5-(acetylamino)-6-pyridin-2-yl-1,2- benzisoxazol-3-yl]carbonyl}amino)-5- cyanobenzoic acid |
| 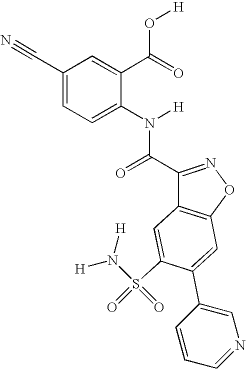 | 2-({[5-(aminosulfonyl)-6-pyridin-3-yl-1,2- benzisoxazol-3-yl]carbonyl}amino)-5- cyanobenzoic acid |
| 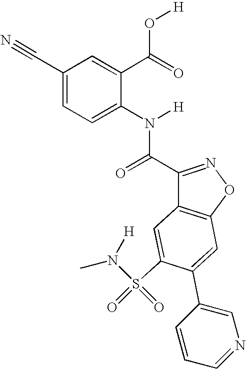 | 5-cyano-2-[({5-[(methylamino)sulfonyl]-6- pyridin-3-yl-1,2-benzisoxazol-3-yl}carbonyl)amino]benzoic acid |
| 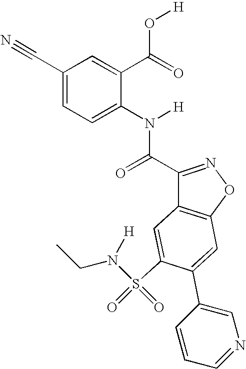 | 5-cyano-2-[({5-[(ethylamino)sulfonyl]-6- pyridin-3-yl-1,2-benzisoxazol-3-yl}carbonyl)amino]benzoic acid |
| 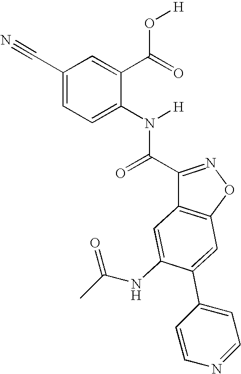 | 2-({[5-(acetylamino)-6-pyridin-4-yl-1,2- benzisoxazol-3-yl]carbonyl}amino)-5- cyanobenzoic acid |
| 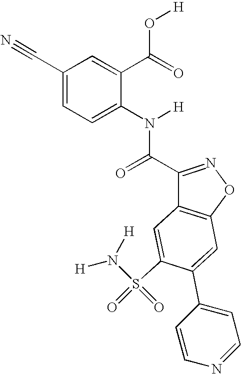 | 2-({[5-(aminosulfonyl)-6-pyridin-4-yl-1,2- benzisoxazol-3-yl]carbonyl}amino)-5- cyanobenzoic acid |
| 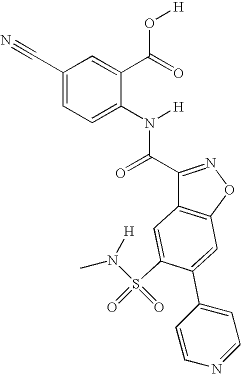 | 5-cyano-2-[({5-[(methylamino)sulfonyl]-6- pyridin-4-yl-1,2-benzisoxazol-3- yl}carbonyl)amino]benzoic acid |
| 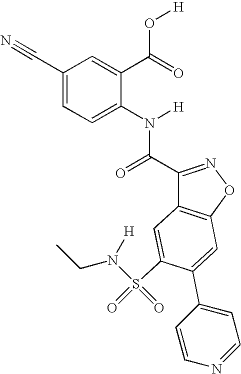 | 5-cyano-2-[({5-[(ethylamino)sulfonyl]-6- pyridin-4-yl-1,2-benzisoxazol-3- yl}carbonyl)amino]benzoic acid |
| 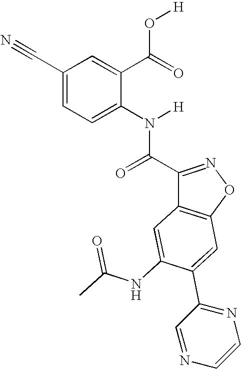 | 2-({[5-(acetylamino)-6-pyrazin-2-yl-1,2- benzisoxazol-3-yl]carbonyl}amino)-5-cyanobenzoic acid |
| 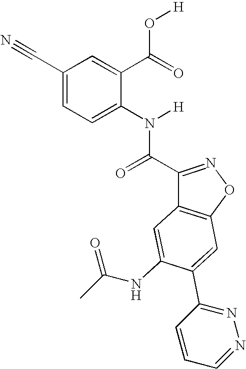 | 2-({[5-(acetylamino)-6-pyridazin-3-yl-1,2- benzisoxazol-3-yl]carbonyl}amino)-5-cyanobenzoic acid |
| 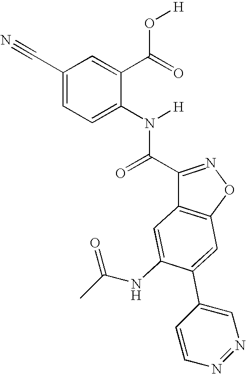 | 2-({[5-(acetylamino)-6-pyridazin-4-yl-1,2- benzisoxazol-3-yl]carbonyl}amino)-5-cyanobenzoic acid |
| 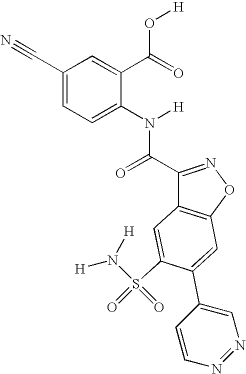 | 2-({[5-(aminosulfonyl)-6-pyridazin-4-yl-1,2- benzisoxazol-3-yl]carbonyl}amino)-5-cyanobenzoic acid |
| 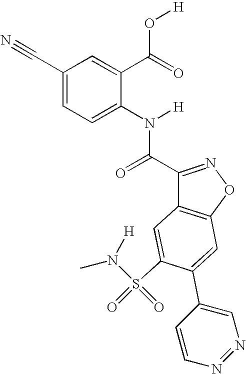 | 5-cyano-2-[({5-[(methylamino)sulfonyl]-6- pyridaziri-4-yl-1,2-benzisoxazol-3- yl}carbonyl)amino]benzoic acid |

Compounds of the invention may also be prepared via a direct coupling shown below.

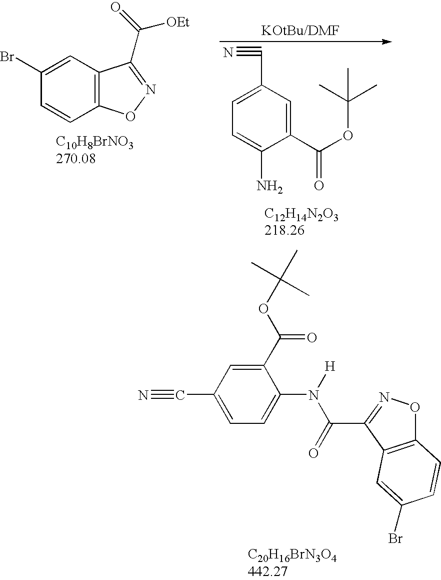

A 50 mL 3-neck round bottom flask was equipped with a J-Kem thermocouple, septum, stir bar and a nitrogen inlet. The flask was heated with a heat gun under nitrogen, cooled to room temperature and tert-butyl 2-amino-5-cyanobenzoate was charged. A 100 mL 3-neck round bottom flask was equipped with a J-Kem thermocouple, septum, stir bar and a nitrogen inlet. The flask was heated with a heat gun under nitrogen, cooled to room temperature and ethyl 5-bromo-1,2-benzisoxazole-3-carboxylate was charged. Anhydrous DMF was added to the flask containing tertbutyl 2-amino-5-cyanobenzoate yielding an orange solution. This solution was cooled with an ice water bath to less than 5° C. and KOtBu was added in 5 portions keeping the temperature below 5° C. The KOtBu additions resulted in a darkening of the solution.

| Initial T | | KOtBu added | Final T |
|---|---|---|---|
| 1^{st} addition | 1.4° C. | about 250 mg | 2.6° C. |
| 2^{nd} addition | 2.5° C. | about 800 mg | 4.4° C. |
| 3^{rd} addition | 2.1° C. | about 800 mg | 4.3° C. |
| 4^{th} addition | 2.0° C. | about 800 mg | 3.7° C. |
| 5^{th} addition | — | about 100 mg | 3.7° C. |

After the KOtBu addition was complete, the solution was allowed to warm to >20 OC, and the solution was held at >20° C. for 1 hour before re-cooling with and ice water bath to <5° C. Anhydrous DMF was added to ethyl 5-bromo-1,2-benzisoxazole-3-carboxylate to yield a pale yellow solution, which was cooled to <5° C. with an ice water bath. Keeping both solutions <5° C, the tert-butyl 2-amino-5-cyanobenzoate solution was transferred via cannula over 5 to 10 minutes to the ester solution and a mild exotherm was noted (Tₘₐₓ 4.5° C). After the addition was complete, the flask was rinsed with DMF, and the mixture was stirred at <5° C. The reaction was assayed by HPLC after 1 hour, and it showed 87% conversion to the desired product (1.8% starting material). The reaction was cal]ed complete at this point and workup was conducted by quenching the reaction into IN HCl and CH₂Cl₂. The CH₂Cl₂ layer was removed, and the aqueous was back extracted with CH₂Cl₂. The combined organics were washed two times with water and dried over MgSO₄. The crude organic was filtered and concentrated on the roto vap at 40° C. to an orange semisolid, which after 1 hour at room temperature on high vacuum weighed 20.97 g, 200 wt %. The assay by ¹H NMR showed a significant amount of DMF in the crude. The sample was purified by adding MeOH and heating the slurry between 50 and 55° C. for 30 minutes followed by cooling to <5° C. The slurry was held at this temperature for 1 hour before isolating the product via filtration using a medium frit. The white product cake was rinsed with MeOH, precooled to <5° C., and dried to constant weight of 8.02 g on high vacuum, 76.4% yield (98.4 area% by HPLC).

### Example 7: R₄ as Isoxazol and Derivatives Thereof

### Example 7.1: Methyl

5-cyano-2- [(E)-2-(5-phenylisoxazol-3-yl)ethenyl]benzoate

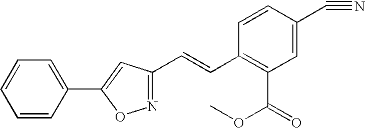

Methyl 2-{[bromo(triphenyl)phosphoranyl]methyl}-5-cyanobenzoate (1.7 g mg, 3.46 mmol) in DMSO (20 mL) followed by the addition of NaH (140 mg, 3.4 mmol). Gas evolution was observed, the reaction was heated to 60° C. for 2 h, then cooled to rt and 5-phenylisoxazole-3-carbaldehyde (500 mg, 2.8 mmol) was added and the reaction was stirred at rt for 2 h. The mixture was diluted with MTBE, washed with H₂O, brine, dried (MgSO₄), filtered and concentrated in vacuo. The residue was purified by silica gel plug (DCM) to afford 996 mg of a Z/E mixture. The solid was dissolved in toluene (40 μL) followed by the addition of thiophenol (32 μL, 0.28 numol) and AIBN (14 mg, 0.08 mmol). The reaction was heated at reflux for 12 h, then concentrated in vacuo. The residue was recrystallized from MeOH to afford 686 mg (72%) of the title compound.

Analytical data

¹H NMR (300 MHz, CDCl₃) δ 8.32 (s, 1H), 8.20 (d, J=16.4 Hz, 1H), 7.87-7.68 (m, 4 H), 7.52-7.50 (m, 3 H), 7.26 (d, J=16.4 Hz, 1H), 5.68 (s, 1H), 4.00 (s, 3 H).

### Example 7.2: 5-Cyano-2-[(E)-2-(5-phenylisoxazol-3-yl)ethenyl]benzoic acid

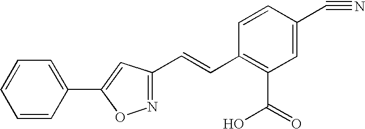

The compound of example 7.1 (250 mg, 0.75 mmol) was dissolved in THF (10 mL) and 6N NaOH (5 mL) was added, the resulting mixture was stirred for 16 h at rt, then diluted with MTBE, washed with IN HCl, H₂O, brine, dried (MgSO₄) filtered and concentrated in vacuo. The residue was recrystallized from MeOH to afford 177 mg (74%) of the title compound.

Analytical data

¹H NMR (400 MHz, DMSO-d₆) δ 13.80 (s, 1H), 8.27 (d, J=1.6 Hz, 1H), 8.14-8.04 (m, 3 H), 7.93 (dd, J=1.7, 8.0 Hz, 2 H), 7.59-7.51 (m, 3 H), 7.44 (s, 1H), 7.36 (d, J=16.4 Hz, 1H).

### Example 7.3: Methyl 5-chloro-2-[(E)-2-(5-phenyl]soxazol-3-yl)ethenyl]benzoate

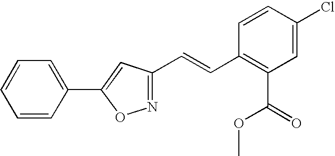

Methyl 2-{[bromo(triphenyl)phosphoranyl]methyl}-5-chlorobenzoate (3.6 g mg, 6.7 mmol) in DMSO (40 mL) followed by the addition of NaH (270 mg, 6.7 mmol). Gas evolution was observed, the reaction was heated to 60° C. for 2 h, then cooled to rt and 5-phenylisoxazole-3-carbaldehyde (838 mg, 4.8 mmol) was added and the reaction was stirred at rt for 2 h. The mixture was diluted with MTBE, washed with H₂O, brine, dried (MgSO₄), filtered and concentrated in vacuo. The residue was purified by silica gel plug (DCM) to afford 996 mg of a Z/E mixture. The solid was dissolved in toluene (40 mL) followed by the addition of thiophenol (100 μL.) and AIBN (14 mg). The reaction was heated at reflux for 12 h, then concentrated in vacuo. The residue was recrystallized from MeOH to afford 1.07 g (69%) of the title compound.

Analytical data

¹H NMR (400 MHz, DMSO-d₆) δ 8.01-7.92 (m, 4 H), 7.88 (d, J=2.3 Hz, 1H), 7.72 (dd, J=2.2, 8.4 Hz, 1H), 7.59-7.53 (m, 3 H), 7.43 (s, 1H), 7.25 (d, J=16.4 Hz, 1 H), 3.91 (s, 3 H).

### Example 7.4: 5-Chloro-2-[(E)-2-(5-phenylisoxazol-3-yl)ethenyl]benzoic acid

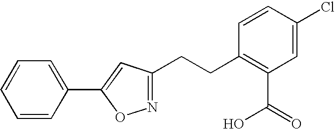

The compound of example 7.3 (312 mg, 0.92 mmol) was dissolved in THF (20 mL) and 6N NaOH (5 mL) was added, the resulting mixture was stirred for 18 h at rt, then diluted with MTBE, washed with IN HCl, H₂O, brine, dried (MgSO₄) filtered and concentrated in vacuo. The residue was recrystallized from MeOH to afford 264 mg (88%) of the title compound.

Analytical data

¹H NMR (400 MHz, DMSO-d₆) 5 13.50 (s, 1H), 8.04 (d, J=16.4 Hz, 1H), 8.02-7.88 (m, 4 H), 7.70-7.67 (m, 1H), 7.58-7.52 (m, 3 H), 7.40 (s, 1), 7.20 (d, J=16.4 Hz, 1H).

Preparation of 5-(2-Fluorophenyl)isoxazole-3-carboxylic acid

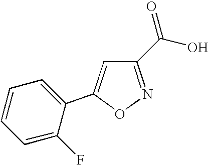

General method A: 1-ethynyl-2-fluorobenzene (1 g, 8.33 mmmol) and dimethyl 2-nitromalonate (1.34 g, 7.58 mmol) were dissolved in mesitylene (10 ml). The solution was heated at 150° C. for about 12 h. Mesitylene was removed in vacuo and the residue was recrystallized from ethanol. 1.22 g (73%) of methyl 5-(2-fluorophenyl)isoxazole-3-carboxylate was yielded as a light brown solid, which was hydrolyzed in MeOH (10 ml) and water (5 ml) by LiOHeH₂O (0.7 g, 16.6 mmol) to afford 0.8 g (70%) of the title compound as a light brown solid.

Analytical data

¹H NMR (400 MHz, DMSO-d₆) δ 8.01 (dt, J=1.7, 7.7 Hz, 1H), 7.84 (m, 1H), 7.49 (m, 1H), 7.43 (dt, J=1.3, 7.7 Hz, 1H), 7.16 (d, J=3.1 Hz, 1H)

### Example 7.5: 5-Cyano-2-({[5-(2-fluorophenyl)isoxazol-3-yl]carbonyl}amino)benzoic acid

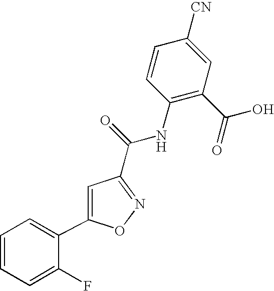

General method B: 5-(2-fluorophenyl)isoxazole-3-carboxylic acid (300 mg, 1.46 mmol), DCM (10 ml) and oxyl chloride (0.5 ml) were place in a flask, followed by the addition of on drop of DMF. The solution was stirred at room temperature for about 50 min. Then the solvent was removed and the residue was pumped high vacuum for 5 min. The residue was dissolved in DCM (5 ml). Tert-butyl-2-amino-5-cyano benzoate (289 mg, 1.32 mmol) was added and followed by the addition of pyridine (0.4 ml). The resulting solution was stirred overnight, then diluted with MTBE (200 ml) and washed with 1N HCl, 1N NaOH, brine, dried (MgSO₄), filtered, and concentrated in vacuo. The residue was recrystallized from MeOH to afford 320 mg (59%) of t-butyl ester as a white solid, 300 mg of which was hydrolyzed by TFA (2 ml) in DCM (8 ml) to afford 225 mg (87%) of a white solid.

General method C: 5-(2-fluorophenyl)isoxazole-3-carboxylic acid (4000 mg, 19.4 mmol) was dissolved in thionyl chloride (40 ml) and the resulting mixture was heated at refluxing temperature overnight. Toluene (30 ml) was added and the solution was concentrated in vacuo. The residue was re-dissolved in DCM (40 ml) followed by the addition of tert-butyl-2-amino-5-cyano benzoate (3530 mg, 16.2 mmol) and pyridine (5.2 ml) and the mixture was stirred overnight. The resulting solution was diluted with MTBE (200 ml), washed with 1N HCl, 1N NaOH, brine and dried (MgSO₄). The solution was concentrated in vacuo and the residue was washed with MeOH to afford 5800 mg (88%) of t-butyl ester as a white solid, which was hydrolyzed by TFA (6 ml) in DCM (10 ml) to afford 5000 mg (100%) of a white solid.

Analytical data

¹H NMR (300 MHz, CDCl₃) δ 12.75 (s, J=Hz, 1H), 8.84 (d, J=8.9 Hz, 1H), 8.43 (d, J=1.9 Hz, 1H), 8.14 (dd, J=1.9, 8.7 Hz, 1H), 8.05 (t, J=7.7 Hz, 1H), 7.65 (m, 1H), 7.47 (m, 2 H), 7.32 (d, J=2.8 Hz, 1H)

### Example 7.6: 5-Bromo-2-({[5-(2-fluorophenyl)isoxazol-3-yl]carbonyl}amino)benzoic acid

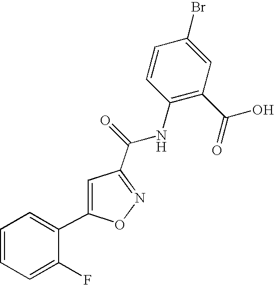

Prepared according to the General method C: 5-(2-fluorophenyl)isoxazole (300 mg, 1.46 nmol) and tert-butyl-2-amino-5-bromobenzoate (329 mg, 1.21 mmol) afforded 360 mg (54%) of t-butyl ester as a white solid, which was hydrolyzed by TFA (2 ml) in DCM (8 ml) to afford 330 mg (100%) of a white solid.

Analytical data

¹H NMR (400 MHz, DMSO-d₆) δ 12.44 (s, 1H), 8.64 (d, J=9.0 Hz, 1H), 8.13 (d, J=2.5 Hz, 1H), 8.04 (dt, J=1.3, 7.7 Hz, 1H), 7.89 (dd, J=2.5, 9.0 Hz, 1H), 7.64 (m, 1H), 7.16 (m, 2 H), 7.27 (d, J=2.8 Hz, 1H)

### Example 7.7: 5-Chloro-2-({[5-(2-fluorophenyl)isoxazol-3-yl]carbonyl}amino)benzoic acid

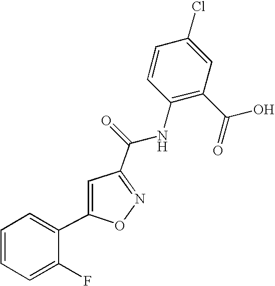

Prepared according to the General method C making non-critical variations.

Analytical data

¹H NMR (300 MHz, DMSO-d₆) δ 8.69 (d, J=9.1 Hz, 1H), 8.04 (dt, J=1.7, 7.7 Hz, 1H), 8.00 (d, J=2.8 Hz, 1H), 7.78 (dd, J=2.6, 8.9 Hz, 1H), 7.65 (m, 1H), 7.46 (m, 2 H), 7.29 (d, J=2.8 Hz, 1H).

Preparation of 1-Ethynyl-2-methylbenzene

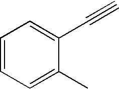

General procedure D: 1-iodo-2-methylbenzene (2000 mg, 9.2 mmol), CuI (699 mg, 3.7 mmol), and dichlorobis(triphenylphosphine) palladium(O) (644 mg, 0.92 mmol) were place in a 100 ml one-necked flask. The system was evacuated and filled with argon several times. THF (30 mil) and triethylamine (30 nml) were added, followed by the addition of ethynyl(trimethyl)silane (9.0 g, 92 mmol). The mixture was stirred at refluxing temperature overnight. Then the resulting mixture was diluted with MTBE (200 ml) and washed with 1N HCl, 1N NaOH, and dried (MgSO₄). The solution was concentrated and the residue was purified by flash chromatography (Heptane/EtOAc=1000/0, 1000/10) to afford 2.2 g of crude trimethyl[(2-methylphenyl)ethynyl]silane as a brown oil. The TMS group was removed by KF (2.0 g, 35 mmol) in MeOH (10 ml) to afford a 310 mg (29% in 2 steps) of 1-ethynyl-2-methylbenzene as brown oil.

Preparation of 5-(2-Methylphenyl)isoxazole-3-carboxylic acid

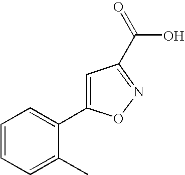

Prepared according to the general method A: 1-ethynyl-2-methylbenzene (310 mg, 2.67 mmol) and dimethyl 2-nitromalonate (430 mg, 2.43 mmol) to afford 200 mg (38%) of methyl 5-(2-methylphenyl)isoxazole-3-carboxylate as a yellow oil, which was hydrolyzed in MeOH (10 ml) and water (5 mnl) by LiOH.H₂O (116 mg, 2.76 mmol) to afford 150 mg (80%) of a light yellow solid.

### Example 7.8: 5-Cyano-2-({[5-(2-methylphenyl)isoxazol-3-yl]carbonyl}amino)benzoic acid

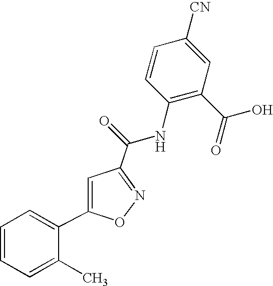

Prepared according to the General method C making non-critical variations with 5-(2-methylphenyl)isoxazole-3-carboxylic acid (140 mg, 0.7 mmol) and tert-butyl-2-amino-5-cyano benzoate (125 mg, 0.6 mmol).

Analytical data

1H NMR (300 MHz, DMSO-d₆) δ 12.78 (s, 1H), 8.86 (d, J=8.9 Hz, 1H), 8.44 (d, J=2.1 Hz, 1H), 8.15 (dd, J=2.1, 8.7 Hz, 1H), 7.82 (d, J=7.7 Hz, 1H), 7.45 (m, 3 H), 7.34 (s, 1H), 2.53 (s, 3 H).

### Example 7.9: 5-Chloro-2-{[(5-phenylisoxazol-3-yl)carbonyl]amino}benzoic acid

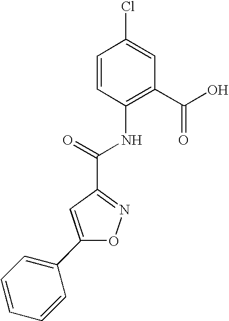

Prepared according to the General method C making non-critical variations.

Analytical data

¹H NMR (300 MHz, DMSO-d₆) δ 12.43 (s, 1H), 8.73 (d, J=9.0 Hz, 1H), 8.02 (d, J=2.6 Hz, 2 H), 7.99 (dd, J=2.1, 5.5 Hz, 1H), 7.78 (dd, J=2.8, 9.0 Hz, 1H), 7.68 (m, 4 H).

### Example 7.10: 5-Cyan -2-{[(5-methylisoxazol-3-yl)carbonyl]amino}benzoic acid

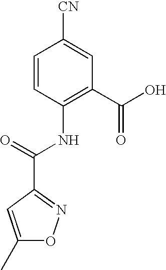

Prepared according to the general method B: 5-methylisoxazole-3-carbonyl chloride (300 mg, 2.1 mmol) and tert-butyl-2-amino-5-cyano benzoate (409 mg, 1.9 mmol) afforded 98 mg (16%) of a white solid, 90 mg of which was hydrolyzed by TFA (2 ml) in DCM (8 ml) to afford 52 mg (74%) of the title compound as a white solid.

Analytical data

¹H NMR (300 MHz, DMSO-d₆) δ 12.64 (s, 1H), 8.82 (d, J=8.9 Hz, 1H), 8.42 (d, J=1.9 Hz, 1H), 8.13 (dd, J=2.1, 8.9 Hz, 1H), 6.76 (s, 1H), 2.52 (s, 3 H).

Preparation of 5-[4-(Trifluoromethyl)phenyl]isoxazole-3-carboxylic acid

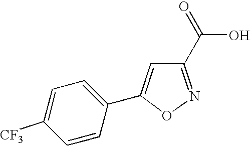

Prepared according to the general method A: 1-ethynyl-2-trifluoromethylbenzene (3.2 g, 18.8 mmol) and dimethyl 2-nitrornalonate (3.03 g, 17.0 mmol) to afford 1.9 g (43%) of methyl 5-(2-trifluoromethylphenyl)isoxazole-3-carboxylate as a yellow solid, which was hydrolyzed in MeOH (40 ml) and water (20 ml) by LiOH.H₂O (883 mg, 21 mmol) to afford 1.8 g (100%) of a white solid.

Analytical data

¹H NMR (400 MHz, DMSO-d₆) δ 14.17 (s, 1H), 8.19 (d, J=8.3 Hz, 2 H), 7.94 (d, J=8.5 Hz, 2 H), 7.64 (s, 1H).

### Example 7.11: 5-Cyano-2-[({5-[4-(trifluoromethyl)phenyl]isoxazol-3-yl}carbonyl)amino]benzoic acid

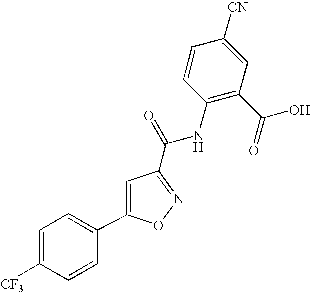

Prepared according to the General method B: 5-[4-(trifluoromethyl_phenyl]isoxazole-3-carboxylic acid (256 mg, .1 nmol) and tert-butyl-2-amino-5-cyano benzoate (196 mg, 0.9 mmol) afforded 190 mg (46%) of t-butyl ester as a white solid, which was hydrolyzed by TFA (2 ml) in DCM (8 ml) to afford 160 mg (96%) of a white solid.

Analytical data

¹H NMR (300 MHz, DMSO-d₆) δ 12.79 (s, 1H), 8.86 (d, J=8.9 Hz, 1H), 8.44 (d, J=2.1 Hz, 1H), 8.23 (d,J=8.1 Hz, 2 H), 8.15 (dd,J=2.1, 8.7 Hz, 1H), 7.97 (d, J=8.3 Hz, 2 H), 7.83 (s, 1H).

### Example 7.12: 5-Bromo-2-[({5-[4-(trifluoromethyl)phenyl]isoxazol-3-yl}carbonyl)amino]benzoic acid

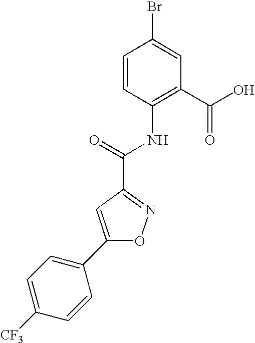

Prepared according to the General method C making non-critical variations.

Analytical data

¹H NMR (400 MHz, DMSO-d₆) δ 12.47 (s, 1H), 8.65 (d, J=8.9 Hz, 1H), 8.23 (d, J=8.1 Hz, 2 H), 8.14 (d, J=2.5 Hz, 1H), 7.95 (d, J=8.3 Hz, 2 H), 7.90 (dd, J=2.5, 8.9 Hz, 1H), 7.79 (s, 1H).

### Example 7.13: 5-Chloro-2-1({5-[4-(trifluoromethyl)phenyl]isoxazol-3-yl}carbonyl)amino]benzoic acid

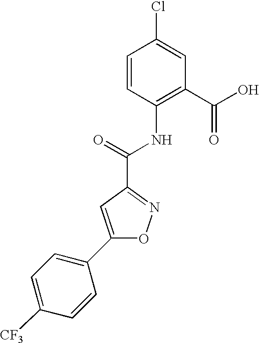

Prepared according to the General method C making non-critical variations.

Analytical data

¹H NMR (400 MHz, DMSO-d₆) δ 12.47 (s, 1H), 8.72 (d, J=8.9 Hz, 1H), 8.23 (d, J=8.1 Hz, 2 H), 8.01 (d, J=2.7 Hz, 1H), 7.96 (d, J=8.5 Hz, 2 H), 7.79 (s, 1H), 7.78 (dd, J=2.7, 8.9 Hz, 1H).

Preparation of 4-Ethynyl-3,5-dimethylisoxazole [36306-jl-69]

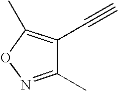

General procedure D: 4-iodo-3,5-dimethylisoxazole (2230 mg, 10 mmol), CuI (720 mg, 3.8 minol), and dichlorobis(triphenylphosphine) palladium(O) (310 mg, 0.47 mmol) were place in a 100 ml one-necked flask. The system was evacuated and filled with argon several times. THF (30 ml) and triethylamine (30 ml) were added, followed by the addition of ethynyl(trimethyl)silane 11 ml, 150 mmol. The mixture was stirred at refluxing temperature overnight. Then the resulting mixture was diluted with MTBE (200 ml) and washed with 1N HCl, 1N NaOH, and dried (MgSO₄). The solution was concentrated and the residue was purified by flash chromatography (Heptane/EtOAc=1000/0, 1000/10) to afford 1.7 g of trimethyl[(2-methylphenyl)ethynyl]silane as a brown oil. The TMS group was removed by KF (1.5 g, 26 mnmol) in MeOH (10 ml) to afford a 690 mg (57% in 2 steps) of 4-Ethynyl-3,5-dimethylisoxazole as brown solid.

Analytical data

¹H NMR (300 MHz, CDCl₃) δ 3.21 (s, 1H), 2.47 (s, 3 H), 2.31 (s, 3 H);

Preparation of 3,5-Dimethyl-4,5′-biisoxazole-3′-carboxylic acid

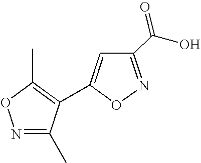

Prepared according to the general method A: 4-Ethynyl-3,5-dimethylisoxazole (690 mg, 5.7 mmol) and dimethyl 2-nitromalonate (918 mg8.2 mmol) to afford 770 mg (67%) of methyl ester as a brown solid, which was hydrolyzed in MeOH (40 ml) and water (20 ml) by LiOH.H₂O (437 mg, 10.4 mmol) to afford 730 mg (100%) of a white solid.

Analytical data

¹H NMR (300 MHz, DMSO-d₆) δ 7.08 (s, 1H), 2.65 (s, 3 H), 2.42 (s, 3 H).

### Example 7.14: 5-Cyano-2-{1(3,5-dimethyl-4,5′-biisoxazol-3′-yl)carbonyl]amino}benzoic acid

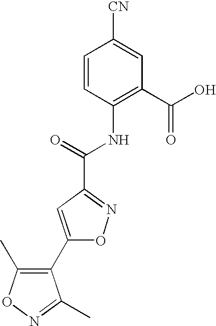

Prepared according to the General method C: 3,5-dimethyl-4,5′-biisoxazole-3′-carboxylic acid (350 mg, 1.68 mnmol) and tert-butyl-2-amino-5-cyano benzoate (306 mg, 1.4 mmol) afforded 380 mg (55%) of t-butyl ester as a white solid, 375 mg of which was hydrolyzed by TFA (2 ml) in DCM (8 ml) to afford 290 mg (90%) of a white solid.

Analytical data

¹H NMR (400 MHz, DMSO-d₆) δ 12.74 (s, 1H), 8.84 (d, J=8.9 Hz, 1H), 8.43 (d, J=2.1 Hz,1H),8.15(dd,J=2.1,8.9 Hz,1H),7.25(s,1H),2.69(s,3H),2.46(s,3 H).

Preparation of 5-(4-Methoxyphenyl)isoxazole-3-carboxylic acid

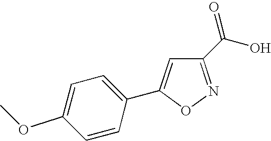

Prepared according to the general method A: 1-ethynyl-4-methoxybenzene (1000 mg, 7.6 mmol) and dimethyl 2-nitromalonate (1220 mg, 6.9 mmol) to affor 1100 mg (68%) of methyl ester as a brown solid, which was hydrolyzed in MeOH (40 ml) and water (20 ml) by LiOH.H₂O (594 mg, 14.2 mmol) to afford 1000 mg (97%) of a brown solid.

Analytical data

¹H NMR (300 MHz, CD₃OD) δ 7.73 (m, 2 H), 6.98 (m, 2 H), 6.81 (s, 1H), 3.85 (s, 3 H).

### Example 7.15: 5-Cyano-2-({[5-(4-methoxyphenyl)isoxazol-3-yl]carbonyl}amino)benzoic acid

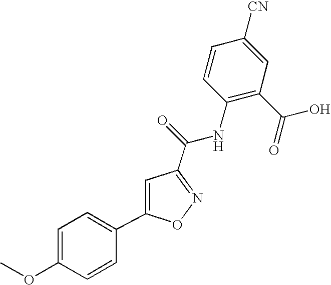

Prepared according to the General method B: 5-(4-methoxyphenyl)isozazole-3-carboxylic acid (300 mg, 1.37 mmol) and tert-butyl-2-amino-5-cyano benzoate (271 mg, 1.25 mmol) afforded 240 mg (46%) of t-butyl ester as a white solid, which was hydrolyzed by TFA (2 ml) in DCM (8 ml) to afford 200 mg (96%) of a white solid.

Analytical data

¹H NMR (300 MHz, DMSO-d₆) δ 12.70 (s, 1H), 8.85 (d, J=8.9 Hz, 1H), 8.42 (d, J=1.9 Hz, 1H), 8.14 (dd, J=1.9, 8.7 Hz, 1H), 7.94 (d, J=8.9 Hz, 2 H), 7.45 (s, 1 H), 7.13 (d, J=8.9 Hz, 2 H), 3.85 (s, 3 H).

### Example 7.16: 5-Bromo-2-({[5-(4-methoxyphenyl)isoxazol-3-yl]carbonyl}amino)benzoic acid

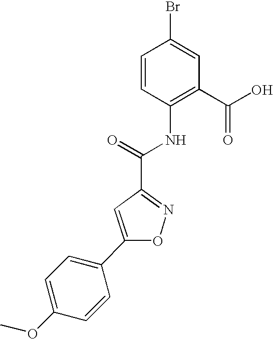

Prepared according to the General method C: 5-(4-methoxyphenyl)isoxazole-3-carboxylic (300 mg, 1.37 mmol) and tert-butyl-2-amino-5-bromo benzoate (309 mg, 1.14 mmol) afforded 450 mg (69%) of t-butyl ester as a white solid, 440 mg of which was hydrolyzed by TFA (2 ml) in DCM (8 ml) to afford 330 mg (85%) of a white solid.

Analytical data

¹H NMR (400 MHz, DMSO-d₆) o 12.39 (s, 1H), 8.66 (d, J=8.9 Hz, 1H), 8.14 (d, J=2.5 Hz, 1H),7.92(m,3H),7.42(s, 1H),7.12(d,J=8.9 Hz,2H),3.85(s,3H).

### Example 7.17: 5-Chloro-2-({[5-(4-methoxyphenyl)isoxazol-3-yl]carbonyl}amino)benzoic acid

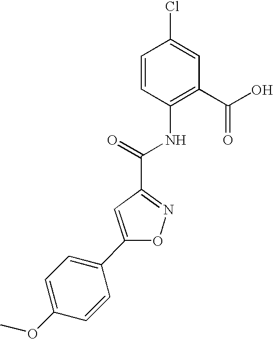

Prepared according to the General method C: making non-critical variations.

Analytical data

¹H NMR (400 MHz, DMSO-d₆) δ 12.41 (s, 1H), 8.72 (d, J=8.9 Hz, 1H), 8.02 (d, J=2.7 Hz, 1H), 7.94 (d, J=8.7 Hz, 2 H), 7.78 (dd, J=2.7, 8.9 Hz, 1H), 7.43 (s, 1 H), 7.12 (d, J=8.9 Hz, 2 H), 3.85 (s, 3 H).

Preparation of 5-(2-Chlorophenyl)isoxazole-3-carboxylic acid

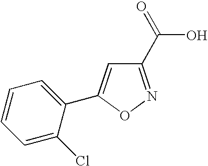

Prepared according to the general method A: 1-ethynyl-2-chlorobenzene (2000 mg, 14.6 mmol) and dimethyl 2-nitromalonate (2356 mg, 13.2 mmol) to afford 1850 mg (59%) of methyl ester as a orange solid, which was hydrolyzed in MeOH (40 ml) and water (20 ml) by LiOH.H₂O (980 mg, 23.4 mmol) to afford 1500 mg (86%) of a orange solid.

Analytical data

¹H NMR (400 MHz, DMSO-d₆) δ 7.97 (m, 1H), 7.71 (m, 1H), 7.58 (m, 2 H), 7.34 (s, 1H).

### Example 7.18: 2-({[5-(2-Chlorophenyl)isoxazol-3-yl]carbonyl}amino)-5-cyanobenzoic acid

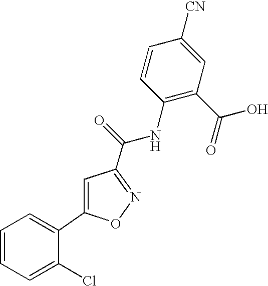

Prepared according to the General method B: making non-critical variations.

Analytical data

¹H NMR (300 MHz, DMSO-d₆) δ 12.75 (s, 1H), 8.84 (d, J=8.9 Hz, 1H), 8.43 (d, J=1.9 Hz, 1H), 8.15 (dd, J=1.7, 8.9 Hz, 1H), 8.01 (dd, J=1.7, 6.8 Hz, 1H), 7.73 (d, J=7.4 Hz, 1H),7.60(m,2H),7.50(s, 1H).

### Example 7.19: 5-Bromo-2-({[5-(2-chlorophenyl)isoxazol-3-yljcarbonyl}amino)benzoic acid

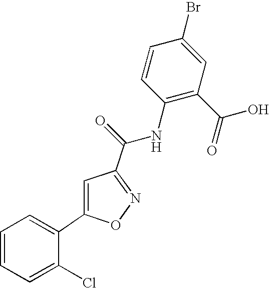

Prepared according to the General method C: making non-critical variations.

Analytical data

¹H NMR (300 MHz, DMSO-d₆) δ 12.46 (s, 1H), 8.64 (d, J=8.9 Hz, 1H), 8.14 (d, J=2.5 Hz, 1H), 8.00 (m, 1H), 7.90 (dd, J=2.5, 9.1 Hz, 1H), 7.73 (m, 1H), 7.60 (m, 2 H), 7.46 (s, 1H).

### Example 7.20: 5-Chloro-2-({[5-(2-chlorophenyl)isoxazol-3-yl]carbonyl}amino)benzoic acid

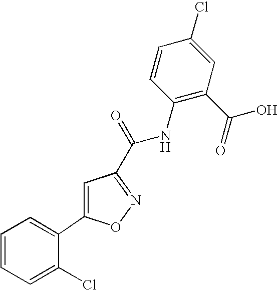

Prepared according to the General method C: making non-critical variations.

Analytical data

¹H NMR (300 MHz, DMSO-d₆) δ 12.40 (s, 1H), 8.70 (d, J=9.1 Hz, 1H), 8.00 (m, 2 H), 7.63 (dd, J=2.6, 9.0 Hz, 1H), 7.72 (m, 1H), 7.60 (m, 2 H), 7.47 (s, 1H).

Preparation of 1-Ethynyl-2-(trifluoromethyl)benzene

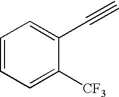

General procedure D: 1-iodo-2-(trifluoromethyl)benzene (2720 mg, 10 mmol), CuI (720 mg, 3.8 mmol), and dichlorobis(triphenylphosphine) palladium(O) (310 mg, 0.47 mmol) were place in a 100 tl one-necked flask. The system was evacuated and filled with argon several times. THF (30 ml) and triethylamine (30 il) were added, followed by the addition of ethynyl(trimethyl)silane 11 ml, 150 mmol. The mixture was stirred at refluxing temperature overnight. Then the resulting mixture was diluted with MTBE (200 mnl) and washed with 1N HCl, 1N NaOH, and dried (MgSO₄). The solution was concentrated and the residue was purified by flash chromatography (Heptane/EtOAc=1000/0, 1000/10) to afford 2.6 g oftrimethyl[(2-methylphenyl)ethynyl]silane as a brown oil. The TMS group was removed by KF (1.9 g, 32 mmol) in MeOH (30 ml) to afford a 1.6 g (94% in 2 steps) of 1-Ethynyl-2(trifluoromethyl)benzene as a brown oil.

Analytical data

¹H NMR (300 MHz, CDCl₃) 6 7.56 (t, J=6.8 Hz, 2 H), 7.40 (t, J=7.3 Hz, 1H), 7.34 (t, J=7.7 Hz, 1H), 3.27 (s, 1H);

Preparation of 5-[2-(Trifluoromethyl)phenyl]isoxazole-3-carboxylic acid

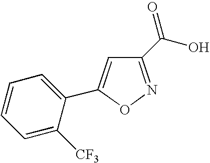

Prepared according to the general method A: 1-Ethynyl-2-(trifluoromethyl)benzene (1600 mg, 9.4 mmol) and dimethyl 2-nitromalonate (1514 mg, 8.56 mmol) to afford 1340 mg (52%) of methyl ester as a brown solid, which was hydrolyzed in MeOH (40 ml) and water (20 ml) by LiOH.H₂O (623 mg, 14.8 minol) to afford 1180 mg (93%) of a brown solid.

Analytical data

¹H NMR (300 MHz, DMSO-d₆) δ 12.41 (s, 1H), 7.99 (d, J=7.4 Hz, 1H), 7.85 (m, 3 H), 7.18 (s, 1H).

### Example 7.21: 5-Cyano-2-[({5-[2-(trifluoromethyl)phenyljisoxazol-3-yl}carbonyl)amino]benzoic acid

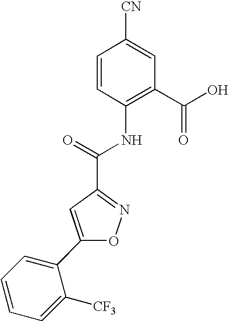

Prepared according to the General method C: 5-[2-(trifluoromethyl)phenyl]isoxazole-3-carboxylic acid (300 mg, 1.17 mmol) and tert-butyl-2-amino-5-cyanobenzoate (212 mg, 1.0 mmol) afforded 420 mg (79%) of t-butyl ester as a white solid, 355 mg of which was hydrolyzed by TFA (2 ml) in DCM (8 mnl) to afford 250 mg (80%) of a white solid.

Analytical data

¹H NMR (400 MHz, DMSO-d₆) δ 12.78 (s, 1H), 8.83 (d, J=8.7 Hz, 1H), 8.44 (d, J=2.1 Hz, 1H), 8.16 (dd, J=2.1, 8.7 Hz, 1H), 8.02 (d, J=7.7 Hz, 1H), 7.91 (m, 3 H), 7.38 (s, 1H)

### Example 7.22: 5-Bromo-2-[({5-[2-(trifluoromethyl)phenyl]isoxazol-3-yl}carbonyl)amino]benzoic acid

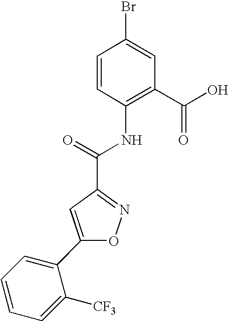

Prepared according to the General method C: making non-critical variations.

Analytical data

¹H NMR (400 MHz, DMSO-d₆) δ 12.47 (s, 1H), 8.64 (d, J=8.9 Hz, 1H), 8.15 (d, J=2.5 Hz, 1H), 8.02 (d, J=7.7 Hz, 1H), 7.90 (m, 3 H), 7.34 (s, 1H).

### Example 7.23: 5-Chloro-2-1({5-[2-(trifluoromethyl)phenyl]isoxazol-3-yl}carbonyl)amino]benzoic acid

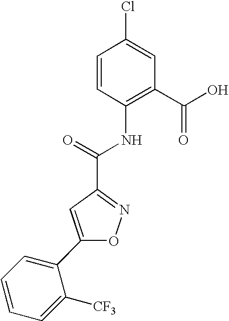

Prepared according to the General method C: making non-critical variations.

Analytical data

¹H NMR (400 MHz, DMSO-d₆) δ 12.47 (s, 1H), 8.70 (d, J=9.1 Hz, 1H), 8.02 (dd, J=5.2, 2.7 Hz, 2 H), 7.91 (m, 4 H), 7.35 (s, 1H).

Preparation of 4-Methyl-5-phenylisoxazole-3-carboxylic acid

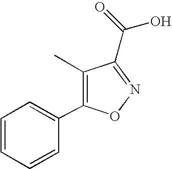

Prepared according to the general method A: prop-1-ynylbenzene (2000 mg, 17.2 mmol) and dimethyl 2-nitromalonate (2770 mg, 15.7 mmol) to afford 930 mg (25%) of methyl ester as a brown solid, which was hydrolyzed in MeOH (20 ml) and water (10 ml) by LiOHeH₂O (540 mg, 12.9 mmol) to afford 910 mg (10%) of a brown solid.

Analytical data

¹H NMR (400 MHz, DMSO-d₆) δ 7.76 (dd, J=1.7, 8.3 Hz, 2 H), 7.59 (m, 3 H), 2.36 (s, 3 H);

### Example 7.24: 5-Cyano-2-{[(4-methyl-5-phenylisoxazol-3-yl)carbonyl]amino}benzoic acid

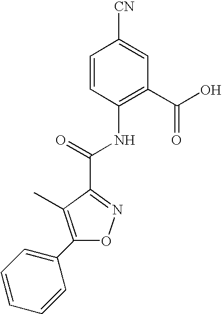

Prepared according to the General method C: 4-methyl-5-phenylisoxazole-3-carboxylic acid (300 mg, 1.48 mmol) and tert-butyl-2-amino-5-cyano benzoate (268 mg, 1.2 mmol) afforded 360 mg (60%) of t-butyl ester as a white solid, 355 mg of which was hydrolyzed by TFA (2 ml) in DCM (8 ml) to afford 290 mg (95%) of a white solid.

Analytical data

¹H NMR (400 MHz, DMSO-d₆) δ 12.68 (s, 1H), 8.86 (d, J=8.7 Hz, 1H), 8.43 (d, J=2.1 Hz, 1H), 8.13 (dd, J=2.1, 8.9 Hz, 1H), 7.80 (dd, J=1.7, 8.3 Hz, 2 H), 7.61 (m, 3 H), 2.45 (s, 3 H).

### Example 7.25: 5-Bromo-2-{[(4-methyl-5-phenylisoxazol-3-yl)carbonyl]amino}benzoic acid

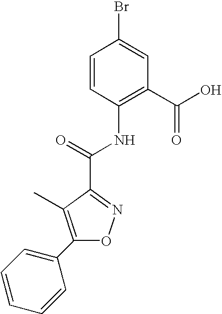

Prepared according to the General method C: making non-critical variations.

Analytical data

¹H NMR (300 MHz, DMSO-d₆) δ 12.37 (s, 1H), 8.67 (d, J=8.9 Hz, 1H), 8.14 (d, J=2.5 Hz, 1H), 7.89 (dd, J=2.6, 9.1 Hz, 1H), 7.81 (m, 2 H), 7.60 (m, 3 H), 2.44 (s, 3 H);

### Example 7.26: 5-C hloro-2-{[(4-methyl-5-phenylisoxazol-3-yl)carbonyl]amino}benzoic acid

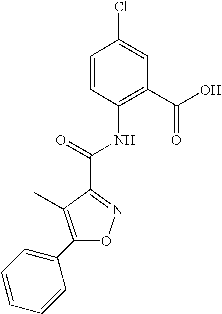

Prepared according to the General method C: making non-critical variations.

Analytical data

¹H NMR (400 MHz, DMSO-d₆) δ 12.36 (s, 1H), 8.72 (d, J=8.9 Hz, 1), 8.01 (d, J=2.7 Hz, 1H), 7.79 (m, 3 H), 7.60 (m, 3 H), 2.44 (s, 3 H).

Preparation of 1-Ethynyl-2-methoxybenzene

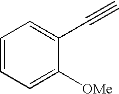

General procedure D: 1-iodo-2-methyoxylbenzene (2000 mg, 8.6 mmol), CuI (651 mg, 3.4 mmol), and dichlorobis(triphenylphosphine) palladium(O) (600 mg, 0.86 mmol) were place in a 100 ml one-necked flask. The system was evacuated and filled with argon several times. TH:F (30 ml) and triethylamine (30 ml) were added, followed by the addition of ethynyl(trimethyl)silane (8.38 g, 86 mmol). The mixture was stirred at refluxing temperature overnight. Then the resulting mixture was diluted with MTBE (200 ml) and washed with 1N HCl, 1N NaOH, and dried (MgSO₄). The solution was concentrated and the residue was purified by flash chromatography (Heptane/EtOAc=1000/0, 1000/10) to afford 2.1 g oftrimethyl[(2-methoxylphenyl)ethynyl]silane as a brown oil. The TMS group was removed by KF (0.94 g, 16 mmol) in MeOH (10 ml) to afford 1.5 g of crude 1-Ethynyl-2-methoxylbenzene as brown oil.

Preparation of 5-(2-Methoxyphenyl)isoxazole-3-carboxylic acid

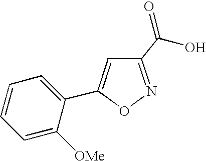

Prepared according to the general method A: 1-Ethynyl-2-methoxylbenzene (1500 mg, 11.4 mmol) and dimethyl 2-nitromalonate (1830 mg, 10.3 mmol) to afford 1100 mg (42%) of methyl ester as a brown solid, which was hydrolyzed in MeOH (20 ml) and water (10 ml) by LiOH.H₂O (600 mg, 14.2 mmol) to afford 850 mg (82%) of a brown solid.

Analytical data

¹H NMR (300 MHz, DMSO-d₆) δ 7.91 (dd, J=1.7, 7.7 Hz, 1H), 7.55 (m, 1H), 7.26 (d, J=7.9 Hz, 1H), 7.14 (t, J=7.7 Hz, 1H), 7.10 (s, 1H), 3.98 (s, 3 H).

### Example 7.27: 5-Cyano-2-({[5-(2-methoxyphenyl)isoxazol-3-yl]carbonyl}amino)benzoic acid

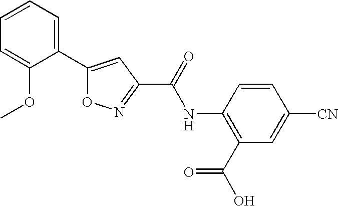

Prepared according to the General method C: 5-(2-methoxyphenyl)isoxazole-3-carboxylic acid (300 mg, 1.37 mmol) and tert-butyl-2-amino-5-cyano benzoate (249 mg, 1.14 mmol) afforded 140 mg (24%) of t-butyl ester as a brown solid, 130 mg of which was hydrolyzed by TFA (2 ml) in DCM (8 ml) to afford 90 mg (80%) of a wnite solid.

Analytical data

¹H NMR (400 MHz, DMSO-d₆) δ 12.71 (s, 1H), 8.84 (d, J=8.7 Hz, 1H), 8.12 (d, J=2.1 Hz, 1H), 8.14 (dd, J=2.1, 8.7 Hz, 1H), 7.94 (dd, J=1.5, 7.8 Hz, 1H), 7.57 (m, 1H), 7.28 (d, J=8.3 Hz, 1H), 7.24 (s, 1H), 7.16 (t, J=8.1 Hz, 1H), 4.00 (s, 3 H).

### Example 7.28: 5-Bromo-2-({[5-(2-methoxyphenyl)isoxazol-3-yl]carbonyl}amino)benzoic acid

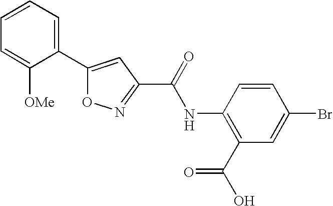

Prepared according to the General method C: making non-critical variations.

Analytical data

¹H NMR (300 MHz, DMSO-d₆) δ 12.42 (s, 1), 8.66 (d, J=9.0 Hz, 1H), 8.14 (d, J=2.5 Hz, 1H), 7.95 (dd, J=1.5, 7.7 Hz, 1H), 7.90 (dd, J=2.4, 8.9 Hz, 1), 7.67 (m, 1H), 7.28 (d, J=8.3 Hz, 1H), 7.22 (s, 1H), 7.16 (t, J=7.7 Hz, 1H), 4.00 (s, 3 H).

### Example 7.29: 5-Chloro-2-({[5-(2-methoxyphenyl)isoxazol-3-ylJcarbonyl}amino)benzoic acid

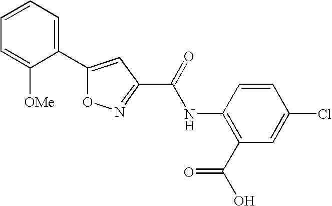

Prepared according to the General method C: making non-critical variations.

Analytical data

¹H NMR (400 MHz, DMSO-d₆) δ 12.43 (s, 1H), 8.72 (d, J=8.9 Hz, 1H), 8.02 (d, J=2.7 Hz, 1H), 7.95 (dd, J=1.7, 7.7 Hz, 1H), 7.79 (dd, J=2.7, 8.9 Hz, 1H), 7.67 (m, 1H), 7.28 (d, J=8.3 Hz, 1H), 7.23 (s, 1H), 7.16 (t, J=7.3 Hz, 1H), 4.00 (s, 3 H).

Preparation of 2-Ethynyl-3-methylthiophene

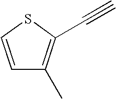

General procedure D: 2-bromo-3-methylthiophene (2000 mg, 11.3 mmol), CuI (861 mg, 4.5 mmol), and dichlorobis(triphenylphosphine) palladium(O) (793 mg, 1.1 mmol) were place in a 100 ml one-necked flask. The system was evacuated and filled with argon several times. THF (30 ml) and triethylamine (30 ml) were added, followed by the addition of ethynyl(trimethyl)silane (11 g, 113 mmol). The mixture was stirred at refluxing temperature overnight. Then the resulting mixture was diluted with MTBE (200 ml) and washed with 1N HCl, 1N NaOH, and dried (MgSO₄). The solution was concentrated and the residue was purified by flash chromatography (Heptane/EtOAc=1000/0, 1000/10) to afford 4.4 g brown oil. The TMS group was removed by KF (3.9 g, 68 nmiol) in MeOH (10 ml) to afford 0.7 g (25% in two steps) of crude 2-Ethynyl-3-methylthiophene as brown oil.

Preparation of 5-(3-methylthien-2-yl)isoxazole-3-carboxylic acid

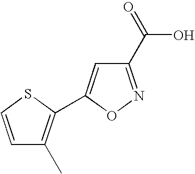

Prepared according to the general method A: 2-ethynyl-3-methylthiophene (700 mg, 5.7 mmol) and dimethyl 2-nitromalonate (923 mg, 5.2 mmmol) to afford 120 mg (9%) of methyl ester as a brown solid, which was hydrolyzed in MeOH (10 ml) and water (5 ml) by LiOH.H₂O (68 mg, 1.6 mmol) to afford 80 mg (71%) of a brown solid.

Analytical data

¹H NMR (400 MHz, DMSO-d₆) δ 7.18 (d, J=5.0 Hz, 1H), 6.75 (d, J=5.2 Hz, 1 H), 6.57 (s, 1), 2.27 (s, 3 H).

### Example 7.30: 5-Cyano-2-({[5-(3-methylthien-2-yl)isoxazol-3-yl]carbonyl}amino)benzoic acid

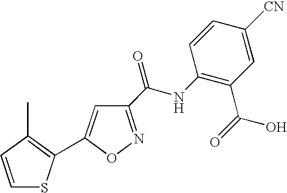

Prepared according to the General method C: 5-(3-methyl-thien-2-yl)isoxazole-3-carboxylic acid (80 mg, 0.38 mmol) and tert butyl-2-amino-5-cyano benzoate (76 mg, 0.35 mmol) afforded 100 mg (64%) of t-butyl ester as a brown solid, which was hydrolyzed by TFA (2 ml) in DCM (8 ml) to afford 60 mg (70%) of a gray solid.

Analytical data

¹H NMR (400 MHz, DMSO-d₆) δ 12.73 (s, 1H), 8.84 (d, J=8.7 Hz, 1H), 8.43 (d, J=2.1 Hz, 1H),8.15(dd,J=2.1,8.9 Hz, 1H), 7.81 (d,J=5.0 Hz, 1H), 7.16 (m,2 H), 2.48 (s, 3 H).

### Example 7.31: 2-{[(5-t-Butylisoxazol-3-yl)carbonyl]amino}-5-cyanobenzoic acid

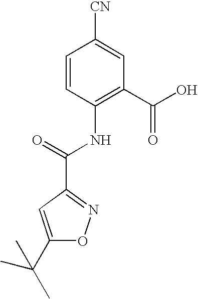

Prepared according to the General method C: 5-(t-butyl)isoxazole-3-carboxylic acid (280 mg, 1.66 mmol) and tert-butyl-2-amino-5-cyano benzoate (301 mg, 1.38 mmol) afforded 301 mg (49%) of t-butyl ester as a white solid, which was hydrolyzed by TFA (2 ml) in DCM (8 ml) to afford 180 mg (71%) of a white solid.

Analytical data

¹H NMR (400 MHz, DMSO-d₆) δ 12.64 (s, 1H), 8.82 (d, J=8.92 Hz, 1H), 8.42 (d, J=2.1 Hz, 1H), 8.12 (dd, J=2.1, 8.7 Hz, 1H), 6.78 (s, 1H), 1.36 (s, 9 H).

Preparation of 5-(3-Cyclohexylpropyl)isoxazole-3-carboxylic acid

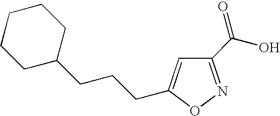

Prepared according to the general method A: pent-4-ynylcyclohexane (2000 mg, 13.3 20 mmol) and dimethyl 2-nitromalonate (2145 mg, 12.1 mmol) to afford 1600 mg (48%) of methyl ester as a brown solid, which was hydrolyzed in MeOH (20 ml) and water (lOml) by LiOHeH₂O (800 mg, 19.1 mmol) to afford 1100 mg (72%) of a brown solid.

Analytical data

¹H NMR (400 MHz, DMSO-d₆) δ 13.87 (s, 1H), 6.60 (s, 1H), 2.78 (t, J=7.5 Hz, 2 H), 1.65 (m, 7 H), 1.18 (m, 6 H), 0.85 (m, 2 H).

### Example 7.32: 5-Cyano-2-({[5-(3-cyclohexylpropyl)isoxazol-3-yl]carbonyl}amino)benzoic acid

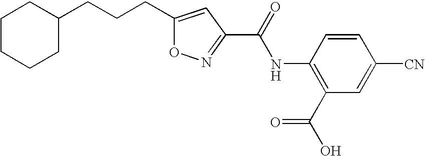

Prepared according to the General method C: 5-(3-cyclohexylpropyl)isoxazole-3-carboxylic acid (300 mg, 1.27 mmol) and tert butyl-2-amino-5-cyano benzoate (230 mg, 1.05 mmol) afforded 500 mg (90%) of t-butyl ester as a brown solid, which was hydrolyzed by TFA (2 ml) in DCM (8 ml) to afford 150 mg (34%) of a brown solid.

Analytical data

¹H NMR (400 MHz, DMSO-d₆) δ 12.62 (s, 1H), 8.82 (d, J=8.9 Hz, 1H), 8.41 (d, J=2.1 Hz, 1H), 8.12 (dd, J=2.1, 8.9 Hz, 1H), 6.78 (s, 1H), 2.83 (t, J=7.5 Hz, 2 H), 1.67 (m 7 H), 1.18 (m, 6 H), 0.85 (m, 2 H).

### Example 7.33: 5-Bromo-2-({[5-(3-cyclolhexylpropyl)isoxazol-3-yl]carbonyl}amino)benzoic acid

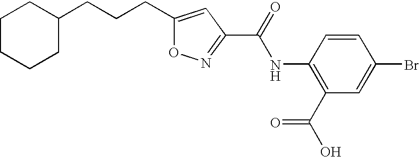

Prepared according to the General method C: making non-critical variations. Analytical data

¹H NMR (400 MHz, DMSO-d₆) δ 12.32 (s, 1H), 8.62 (d, J=8.9 Hz, 1H), 8.13 (d, J=2.5 Hz, 1H), 7.88 (dd, J=2.5, 8.9 Hz, 1), 6.75 (s, 1H), 2.83 (t, J=7.5 Hz, 2 H), 1.66(m,7H), 1.18(m,6H),0.85(m,2H).

### Example 7.34: 5-Chloro-2-(([5-(3-cyclohexylpropyl)isoxazol-3-yl]carbonyl}amino)benzoic acid

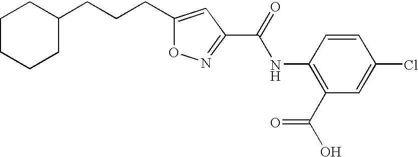

Prepared according to the General method C: making non-critical variations.

Analytical data

¹H NMR (400 MHz, DMSO-d₆) δ 12.31 (s, 1H), 8.68 (d, J=8.9 Hz, 1H), 8.00 (d, J=2.7 Hz, 1H), 7.76 (dd, J=2.5, 8.9 Hz, 1H), 6.75 (s, 1H), 2.83 (t, J=7.5 Hz, 2 H), 1.65 (m, 7 H), 1.18 (m, 6 H), 0.85 (m, 2 H).

Preparation of 5-(3-Phenylpropyl)isoxazole-3-carboxylic acid

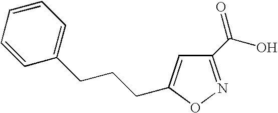

Prepared according to the general method A: pent-4-ynylbenzene (3000 mg, 20.8 mmol) and dimethyl 2-nitromalonate (3347 mg, 18.9 mmol) to afford 3800 mg (82%) of methyl ester as a brown solid, which was hydrolyzed in MeOH (20 ml) and water (10 ml) by LiOH.H₂O (1540 mg, 36.7 mmol) to afford 2600 mg (92%) of a brown solid.

Analytical data

¹H NMR (300 MHz, DMSO-d₆) δ 13.98 (s, 1H), 7.26 (m, 5 H), 6.64 (s, 1H), 2.81 (t, J=7.7 Hz, 2 H), 2.64 (t, J=7.3 Hz, 2 H), 1.98 (pent, J=7.5 Hz, 2 H).

### Example 7.35: 5-Cyano-2-({[5-(3-phenylpropyl)isoxazol-3-yl]carbonyl}amino)benzoic acid

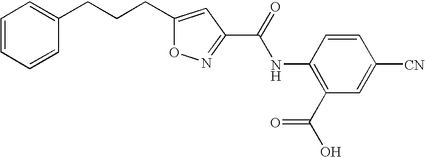

Prepared according to the General method C: 5-(3-phenylpropyl)isoxazole-3-carboxylic acid (300 mg, 1.30 mmol) and tert-butyl-2-amino-5-benzoate (236 mg, 1.08 trmol) afforded 320 mg (57%) of t-butyl ester as a white solid, 310 mg of which was hydrolyzed by TFA (2 ml) in DCM (8 ml) to afford 170 mg (63%) of a white solid.

Analytical data

¹H NMR (400 MHz, DMSO-d₆) δ 12.71 (s, 1H), 8.84 (d, J=8.7 Hz, 1H), 8.12 (d, J=2.1 Hz, 1H), 8.14 (dd, J=2.1, 8.7 Hz, 1H), 7.94 (dd, J=1.5, 7.8 Hz, 1H), 7.57 (m, 1H), 7.28 (d, J=8.3 Hz, 1H), 7.24 (s, 1H), 7.16 (t, J=8.1 Hz, 1H), 4.00 (s, 3 H).

### Example 7.36: 5-Bromo-2-({[5-(3-phenylpropyl)isoxazol-3-ylJ carbonyl}amino)benzoic acid

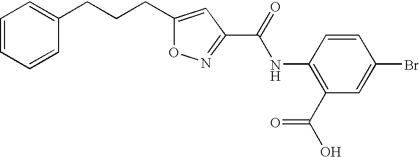

Prepared according to the General method C: making non-critical variations.

Analytical data

¹H NMR (300 MHz, DMSO-d₆) δ 12.32 (s, 1H), 8.63 (d, J=9.0 Hz, 1H), 8.13 (d, J=2.5 Hz, 1H), 7.88 (dd, J=2.5, 8.9 Hz, 1H), 7.25 (m, 5 H), 6.79 (s, 1H), 2.87 (t, J=7.3 Hz, 2 H), 2.66 (t, J=7.9 Hz, 2 H), 2.00 (pent, J=7.5 Hz, 2 H).

### Example 7.37: 5-Chloro-2-({[5-(3-phenylpropyl)isoxazol-3-yl]carbonyl}amino)benzoic acid

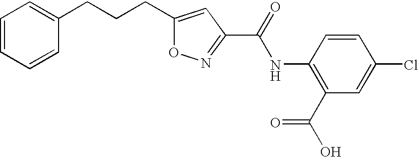

Prepared according to the General method C: making non-critical variations.

Analytical data

¹H NMR (400 MHz, DMSO-d₆) δ 12.31 (s, 1H), 8.68 (d, J=9.1 Hz, 1H), 8.00 (d, J=2.7 Hz, 1H), 7.77 (dd, J=2.7, 9.1 Hz, 1H), 7.26 (m, 5 H), 6.80 (s, 1H), 2.86 (t, J=7.5 Hz, 2 H), 2.66 (t, J=7.9 Hz, 2 H), 2.00 (pent, J=7.7 Hz, 2 H).

Preparation of N-(2-ethynylphenyl)-4-(trifluoromethoxy)benzenesuffonamide

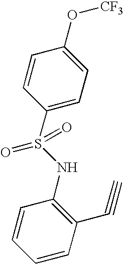

2-[(trimethylsilyl)ethynyl]aniline (2000 mg, 10.6 mmol) and 4(trifluoromethoxy)benzenesulfonyl chloride (2500 mg, 9.6 mmol) were dissolved in DCM (10 ml). Pyridine (0.4 ml) was added. And the n the solution was stirred at room temperature over night. The resulting solution was stirred overnight, then diluted with MTBE (200 ml) and washed with 1N HCl, 1N NaOH, brine, dried (MgSO₄), filtered, and concentrated in vacuo. The residue was recrystallized from MeOH to afford 3340 mg (76%) of 4-(trifluoromethoxy)-N-{2[(trimethylsilyl)ethynyl]phenyl}benzenesulfonamide as a white solid, which was deprotected by KF (1410 mg, 24.3 mmol) in MeOH (20 ml) to afford 2440 mg (99%) of N-(2-ethynylphenyl)-4-(trifluoromethoxy)benzenesulfonamide as a yellow solid.

Analytical data

¹H NMR (400 MHz, CDCl₃) 8 7.67 (m, 2 H), 7.45 (d, J=8.3 Hz, 1H), 7.19 (m, 2 H), 7.08 (m, 2 H), 6.92 (dt, J=1.2, 7.7 Hz, 1H), 3.18 (s, 1H);

### Example 7.38: 5-12-({[4(Trifluoromethoxy)phenyl]suffonyl}amino)phenyl]isoxazole-3-carboxylic acid

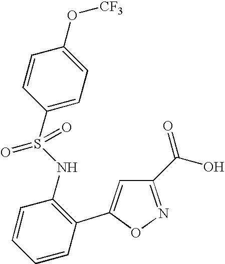

Prepared according to the general method A: N-(2-ethynylphenyl)-4(trifluoromethoxy)benzenesulfonamide (2440 mg, 7.16 mmol) and dimethyl 2-nitromalonate (1150 mg, 6.5 mmol) to afford 1800 mg (57%) of methyl ester as a yellow solid, which was hydrolyzed in MeOH (10 ml) and water (5ml) by LiOH.H₂O (513 mg, 12.22 mmol) to afford 1500 mg (86%) of a brown solid.

Analytical data

¹H NMR (400 MHz, DMSO-d₆) δ 10.26 (s, 1H), 7.82 (dd, J=1.5, 7.1 Hz, 1H), 7.74 (d, J=8.7 Hz, 2 H), 7.49 (m, 4 H), 7.14 (s, 1H), 7.08 (d, J=7.3 Hz, 1H).

### Example 7.39: 5-Cyano-2-f({5-12-({[4-(trifluoromethoxy)phenyl]sulfonyl}amino)phenyl]isoxazol-3-yl}carbonyl)amino]benzoic acid

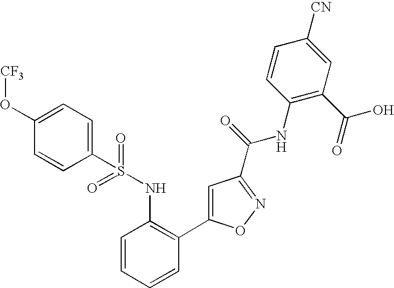

Prepared according to the General method C: 5-[2-({[4(Trifluoromethoxy)phenyl]sulfonyl}amino)phenyl]isoxazole-3-carboxylic acid (300 mg, 0.7 mmol) and tert-butyl-2-amino-5-cyano benzoate (127 mg, 0.58 mmol) afforded 62 mg (17%) of t-butyl ester as a white solid, 310 mg of which was hydrolyzed by TFA (2 ml) in DCM (8 ml) to afford 36 mg (40%) of a yellow solid.

Analytical data

¹H NMR (300 MHz, DMSO-d₆) δ 12.76 (s, 1), 10.32 (s, 1H), 8.87 (d, J=8.7 Hz, 1H), 8.45 (d, J=2.1 Hz, 1H), 8.17 (2.1, J=8.7 Hz, 1H), 7.86 (i, 1H), 7.72 (m, 2 H), 7.50 (m, 4 H), 7.31 (s, 1H), 7.12 (m, 1H).

### Example 7.40: 5-Chloro-2-[({5-[2-({14-(trifluoromethoxy)phenyl]sulfonyl}amino)phenyl]isoxazol-3-yl}carbonyl)amino]benzoic acid

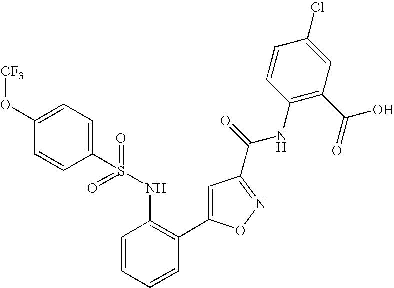

Prepared according to the General method C: making non-critical variations.

Analytical data

¹H NMR (300 MHz, DMSO-d₆) δ 12.40 (s, 1H), 10.32 (s, 1H), 8.73 (d, J=9.0 Hz, 1H), 8.04 (d, J=2.6 Hz, 1H), 7.86 (dd, J=1.7, 6.8 Hz, 1H), 7.80 (dd, J=2.6, 9.0 Hz, 1H), 7.72 (d, J=8.9 Hz, 2 H), 7.50 (m, 4 H), 7.28 (s, 1H), 7.11 (dd, J=1.7, 7.2 Hz, 1H).

Preparation of 5-Ethynyl-1,3-benzodioxole

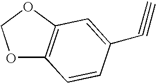

Prepared according to the general method D: 5-iodo-1,3-benzodioxole (5 g, 20.3 mmol) and ethynyl(trimethyl)silane (2.6 g, 26.42 mmol) afforded 4.4 g (99%) of (1,3-benzodioxol-5-ylethynyl)(trimethyl)silane as a yellow oil, which was de-protected by KF(3.5 g, 60.6 mmol) to afford 3.2 g (100%) yellow oil.

Analytical data

¹H NMR (300 MHz, CDCl₃) 5 7.05 (dd, J=1.5, 7.9 Hz, 1H), 6.95 (d, J=1.5 Hz, 1 H), 6.77 (d, J=8.1 Hz, 1H), 6.00 (s, 2 H), 2.99 (s, 1H);

Preparation of 5-(1,3-Benzodioxol-5-yl)isoxazole-3-carboxylic acid

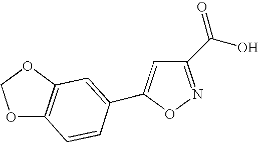

Prepared according to the general method A: 5-ethynyl-1,3-benzodioxole (3200 mg, 21.9 mmol) and dimethyl 2-nitromalonate (3527 mg, 19.9 mmol) to afford 3600 mg (72%) of methyl ester as a brown solid, 2000 mg of which was hydrolyzed in MeOH (10 ml) and water (5ml) by LiOH.H₂O (1020 mg, 24.3 mmol) to afford 1500 mg (80%) of a white solid.

Analytical data

¹H NMR (300 MHz, DMSO-d₆) δ 7.52 (d, J.=1.3 Hz, 1H), 7.49 (d, J=8.1 Hz, 1H), 7.80 (s, 1H), 7.09 (d, J=8.1 Hz, 1H), 6.13 (s, 2 H);

### Example 7.41: 2-({[5-(1,3-Benzodioxol-5-yl)isoxazol-3-yl]carbonyl}amino)-5-cyanobenzoic acid

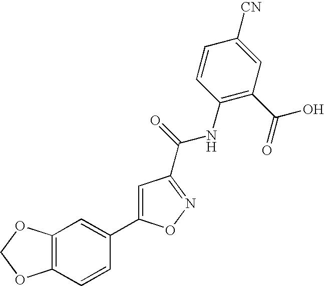

Prepared according to the General method C: 5-(1,3-Benzodioxol-5-yl)isoxazole-3-carboxylic acid (300 mg, 1.28 mmol) and tert-butyl-2-arniino-5-cyano-benzoate (233 mg, 1.07 mmol) afforded 220 mg (47%) of t-butyl ester as a white solid, 310 mg of which was hydrolyzed by TFA (2 ml) in DCM (8 ml) to afford 120 mg (63%) of a brown solid.

Analytical data

¹H NMR (300 MHz, DMSO-d₆) δ 12.68 (s, 1H), 8.84 (d, J=8.7 Hz, 1H), 8.42 (d, J=1.9 Hz, 1H), 8.13 (dd, J=2.1, 8.7 Hz, 1H), 7.67 (d, J=1.5 Hz, 1H), 7.63 (dd, J=1.7, 8.1 Hz, 1H), 7.48 (s, 1H), 7.11 (d,J=8.10 Hz, 1H), 6.15 (s, 2 H).

### Example 7.42: 2-({[5-(1,3-Benzodioxol-5-yl)isoxazol-3-yl]carbonyl}amino)-5-bromobenzoic acid

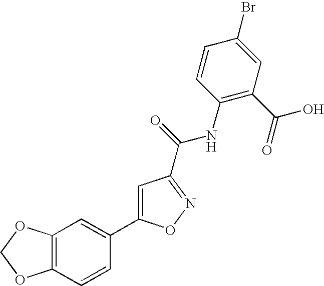

Prepared according to the General method C: making non-critical variations.

Analytical data

¹H NMR (300 MHz, DMSO-d₆) δ 12.38 (s, 1H), 8.65 (d, J=9.1 Hz, 1H), 8.14 (d, J=2.5 Hz, 1H), 7.89 (dd, J=2.6, 9.0 Hz, 1H), 7.67 (d, J=1.7 Hz, 1H), 7.63 (dd, J=1.7, 8.1 Hz, 1H), 7.45 (s, 1H), 7.11 (d, J=8.1 Hz, 1H), 6.15 (s, 2 H).

Preparation of 5-(4-{[(5-Methylisoxazol-3-yl)aminolsulfonyl}phenyl)isoxazole-3-carboxylic acid

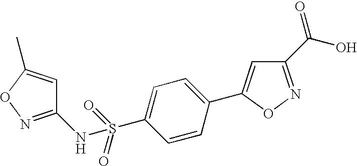

Prepared according to the general method A: 4-ethynyl-N-(5-methyl-3isoxazoyl)benzenesulfonamide (lOOOmg, 3.8 mmol) and dimethyl 2-nitromalonate (613 mg, 3.47 mmol) to afford 1200 mg (53%) of methyl ester as a brown solid, which was hydrolyzed in MeOH (20 ml) and water (10 ml) by LiOH9H₂O (416 mg, 9.9 mmol) to afford 940 mg (82%) of a brown solid.

Analytical data

¹H NMR (300 MHz, DMSO-d₆) δ 11.64 (s, 1H), 8.19 (d, J=8.5 Hz, 2 H), 8.01 (d, J=8.5 Hz, 2 H), 7.61 (s, 1H), 6.18 (s, 1H), 2.30 (s, 3 H).

### Example 7.43: 5-Bromo-2-({[5-(4-{[(5-methylisoxazol-3-yl)aminol suffonyl}phenyl)isoxazol-3-yl]carbonyl}amino)benzoic acid

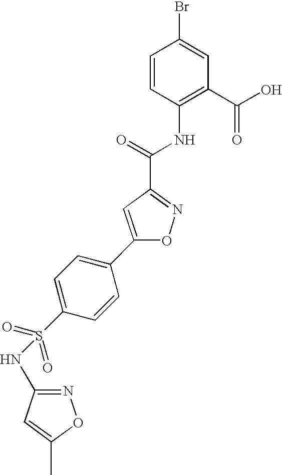

Prepared according to the General method C: 5-(4-{[(5-Methylisoxazol-3-yl)amino]sulfonyl}phenyl)isoxazole-3-carboxylic acid (300 mg, 0.86 mmol) and tert-butyl-2-amimo-5-bromo benzoate (194 mg, 0.72 mmol) afforded 60 mg (12%) oftbutyl ester as a brown solid, which was hydrolyzed by TFA (2 ml) in DCM (8 ml) to afford 30 mg (55%) of a brown solid.

Analytical data

¹H NMR (400 MHz, DMSO-d₆) δ 12.47 (s, 1H), 11.66 (s, 1H), 8.65 (d, J=8.9 Hz, 1H), 8.24 (d, J=8.7 Hz, 2 H), 8.15 (d, J=2.7 Hz, 1H), 8.03 (d, J=8.7 Hz, 2 H), 7.91 (dd, J=2.5, 9.1 Hz, 1H), 7.76 (s, 1H), 2.31 (s, 3 H).

Preparation of 5-(3-Methoxyphenyl)isoxazole-3-carboxylic acid

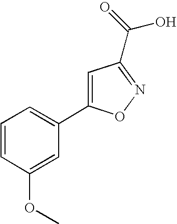

Prepared according to the general method A: 1-ethynyl-3-methoxybenzene (5000 mg, 37.9 mmol) and diethyl 2-nitromalonate (7060 mg, 34.4 mmol) to afford 4100 mg (48%) of ethyl ester as a brown solid, 1000 mg of which was hydrolyzed in MeOH (20 ml) and water (I mi) by LiOH.H₂O (1000 mg, 23.8 mmol) to afford 860 mg (92%) of a brown solid.

Analytical data

¹H NMR (400 MHz, DMSO-d₆) δ 7.50 (m, 4 H), 7.12 (ddd, J=1.0, 2.5, 8.1 Hz, 1H), 3.85 (s, 3 H).

### Example 7.45: 5-Cyano-2-({[5-(3-methoxyphenyl)isoxazol-3-ylIcarbonyl}amino)benzoic acid

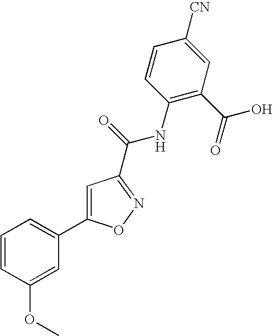

Prepared according to the General method C: 5-(3-methoxyphenyl)isoxazole-3-carboxylic acid (340 mg, 1.55 mmol) and tert butyl-2-amino-5-cyano benzoate (372 mg, 1.71 mmol) afforded 510 mg (87%) of t-butyl ester as a white solid, 500 mg of which was hydrolyzed by TFA (2 ml) in DCM (8 ml) to afford 320 mg (100%) of a white solid.

Analytical data

¹H NMR (400 MHz, DMSO-d₆) δ 12.73 (s, 1H), 8.85 (d, J=8.7 Hz, 1H), 8.42 (d, J=1.3 Hz, 1H), 8.14 (dd, J=1.5, 8.7 Hz, 1H), 7.66 (s, 1H), 7.65 (m, 2 H), 7.49 (t, J=7.9 Hz, 1H), 7.13 (d, J=7.9 Hz, 1H), 3.86 (s, 3 H).

Preparation of 5-(2-Bromophenyl)isoxazole-3-carboxylic acid

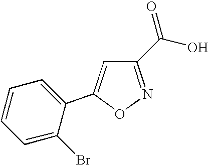

Prepared according to the general method A: 1-bromo-2-ethynylbenzene (2000 mg, 11.0 mmol) and diethyl 2-nitromalonate (2050 mg, 10.0 mmol) to afford 2800 mg (96%) of ethyl ester as a brown oil, which was hydrolyzed in MeOH (20 ml) and water (10 ml) by LiOH.H₂O (1000 mg, 23.8 mmol) to afford 1700 mg (93%) of a white solid.

Analytical data

H NMR (400 MHz, DMSO-d₆) δ 7.87 (dd, J=1.5, 8.3 Hz, 2 H), 7.59 (dt, J=1.5, 7.5 Hz, 1H), 7.51 (m, 1H), 7.34 (s, 1H).

### Example 7.46: 2-({[5-(2-Bromophenyl)isoxazol-3-yl]carbonyl}amino)-5-cyanobenzoic acid

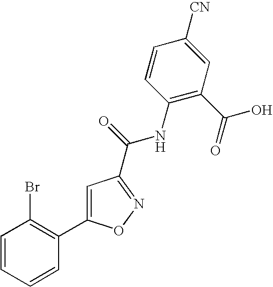

Prepared according to the General method C: 5-(2-bromophenyl)isoxazole-3-carboxylic acid (1900 mg, 7.1 mmol) and tert-butyl-2-amino-5-cyano benzoate (1700 mg, 7.8 mmol) afforded 690 mg (20%) of t-butyl ester which was hydrolyzed by TFA (2 ml) in DCM (8 ml) to afford 38 mg (87%) of a white solid.

Analytical data

¹H NMR (300 MHz, DMSO-d₆) δ 12.81 (s, 1H), 8.85 (d, J=8.7 Hz, 1H), 8.44 (d, J=2.1 Hz, 1H), 8.16 (dd, J=2.1, 8.9 Hz, 1H), 7.91 (nm, 2 H), 7.63 (dt, J=1.3, 7.5 Hz, 1H), 7.65 (dd, J=1.7, 7.5 Hz, 1H), 7.61 (s, 1H).

### Example 7.47: 2-({[5-(1,1 ′-Biphenyl-2-yl)isoxazol-3-yl]carbonyl}amino)-5-cyanobenzoic acid

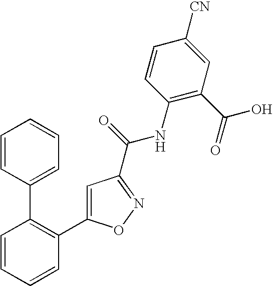

5-(2-bromophenyl)isoxazole-3-carboxylic acid (300 mg, 0.64 mmol), sodium carbonate (259 mg, 2.44 mmol), benzyl boronic acid (86 mg, 0.71 mmol) and tetrakis(triphenylphosphine) palladium(O) (37 mg, 0.032 mmmol) were placed in a 100 ml one-necked flask. The system was evacuated and filled with argon several times. Then THF (50 ml) and distil]ed water (5 ml) were added. The mixture was stirred at refluxing temperature overnight. Then the resulting solution was loaded directly on silica gel and purified by silica gel chromatography (EtOAc/Heptane=1/10) to afford 210 mg (70%) of the advanced t-butyl ester as a white solid, which was hydrolyzed by TFA (2 ml) in DCM (8 ml) to afford 140 mg (76%) of a white solid.

Analytical data

¹H NMR (300 MHz, DMSO-d₆) δ 12.61 (s, 1H), 8.76 (d, J=8.9 Hz, 1H), 8.40 (d, J=2.1 Hz, 1H), 8.11 (dd, J=2.1, 8.9 Hz, 1H), 7.91 (dd, J=1.1, 7.4 Hz, 1H), 7.64 (m, 2 11), 7.49 (dd, J=1.5, 7.5 Hz, 1H), 7.44 (mn, 1H), 7.43 (d, J=2.1 Hz, 2 H), 7.29 (m, 2 H), 6.45 (s, 1H).

Preparation of 1-Ethynyl-2-[(4-methoxybenzyl)oxylbenzene

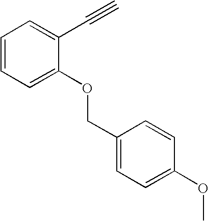

2-ethynylphenol (75 g, 341 mmol), Nal (90 g, 682 minol) and K₂CO₃ (63 g, 682 mmol) were place in a 1 L flask. Acetonitrile (500 ml) was added, followed by the addition of 4-methoxy benzyl chloride (50 g, 319 mmol). The mixture was stirred at room temperature for about 12 h. After filtration, the solution was washed with 1N NaOH, 1N HCl, and dried (MgSO₄). The solution was concentrated to yield 101 g (87%) of 1iodo-2-[(4-methoxyben as a yellow solid. 1-iodo-2-[(4-methoxybenzyl)oxy]benzene (61 g, 180 mmol), CuI (6.85 g, 35.9 mmol), and dichlorobis(triphenylphosphine) palladium(O) (12.6 g, 18.0 mmol) were placed in a 1000 ml one-necked flask. The system was evacuated and filled with argon several times. TBF (250 ml) and diethylamine (250 ml) were added, followed by the addition of ethynyl(trimethyl)silane (22.8 g, 233 mmol. The mixture was stirred at refluxing temperature overnight. Then the resulting mixture was diluted with MTBE (1000 ml) and washed with 1N HCl, 1N NaOH, and dried (MgSO₄). The solution was concentrated and the residue was purified by flash chromatography (Heptane/EtOAc=1000/0, 1000/10) to afford 42 g (76%) of crude ({2-[(4-methoxybenzyl)oxy]phenyl}ethynyl)(trimethyl)silane as a yellow oil. The TMS group was removed by KF (12.0 g, 203 mmol) in MeOH (100 ml) to afford a 28 g (87%) of 1-Ethynyl-2-[(4-methoxybenzyl)oxy]benzene as a yellow solid.

Analytical data

1H NMR (300 MHz, CDCl₃) 6 7.50 (dd, J=1.9, 7.9 Hz, 1), 7.42 (d, J=8.7 Hz, 2 H), 7.29 (m, 1H), 6.93 (m, 5 H), 5.15 (s, 2 H), 3.83 (s, 3 H), 3.32 (s, 1H).

Preparation of Ethyl 5-(2-hydroxyphenyl)isoxazole-3-carboxylate

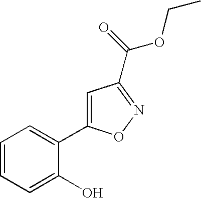

1-Ethynyl-2-[(4-methoxybenzyl)oxy]benzene (28 g, 118 mmol) and diethyl 2-nitromalonate (22.3 g, 107 mmol) were dissolved in mesitylene (20 ml). The solution was heated at 150° C. for about 12 h. Mesitylene was removed in vacuo and the residue was recrystallized from ethanol. 36 g (87%) of ethyl 5-{2-[(4-methoxybenzyl)oxy]phenyl}isoxazole-3-carboxylate was yielded as a light brown solid, 16 g of which was hydrolyzed in DCM (50 ml) TFA (6 mmol) to afford 10.6 g (100%) of a white solid.

Analytical data

¹H NMR (400 MHz, DMSO-d₆) δ 10.81 (s, 1H), 8.83 (dd, J=1.7, 7.9 Hz, 1H), 7.88 (m, 1 H), 7.14 (s, 1 H), 7.07 (dd, J=0.8, 8.3 Hz, 1 H), 6.99 (m, 1 H), 4.40 (tetra, J=7.1 Hz, 2 H), 1.35 (t, J=7.3 Hz, 3 H).

Preparation of 5-(2—Isopropoxyphenyl)isoxazole-3-carboxylic acid

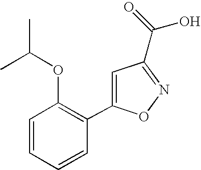

General method A: Ethyl 5-(2-hydroxyphenyl)isoxazole-3-carboxylate (300 mg, 1.3 mmol) and triphenylphosphine (337 mg, 1.3 mmol) were dissolved in THF (5 ml). Propan-2-ol (77 mg, 1.3 mmol) was added, followed by the addition of DEAD (224 mg, 1.3 mmol). The mixture was shaken over night. The resulting solution was loaded on silica gel and purified by flashing chromatography ((Heptane/EtOAc=1000/10, 1000/20) to afford 300 mg (51%) of ethyl 5-(2-isopropoxyphenyl)isoxazole-3-carboxylate as a white solid, which was hydrolyzed in MeOH (10 ml) and water (5 ml) by LiOH.H₂O (137 mg, 3.3 mmol) to afford 270 mg (100%) of 5-(2-Isopropoxyphenyl)isoxazole-3-carboxylic acid as a white solid.

Analytical data

¹H NMR (300 MHz, DMSO-d₆) δ 14.05 (s, 1H), 7.91 (dd, J=1.5, 7.9 Hz, 1H), 7.51 (m, 1H), 7.26 (d, J=8.3 Hz, 1H), 7.11 (d, J=7.5 Hz, 1H), 7.08 (s, 1H), 4.86 (hept, J=6.0 Hz, 1H), 1.37 (d, J=6.0 Hz, 6 H).

### Example 7.48: 5-Cyano-2-({[5-(2-isopropoxyphenyl)isoxazol-3-yl]carbonyl}amino)benzoic acid

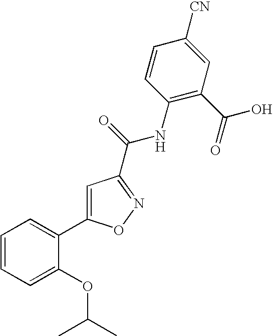

General method B: 5-(2—Isopropoxyphenyl)isoxazole-3-carboxylic acid (270 mg, 1.1 mmol) was dissolved in thionyl chloride (20 ml) and the resulting mixture was heated at refluxing temperature overnight. Toluene (30 ml) was added and the solution was concentrated in vacuo. The residue was re-dissolved in DCM (40 ml) followed by the addition of tert-butyl-2-amino-5-cyano benzoate (218 mg, 1.0 mmol) and pyridine (0.4 ml) and the mixture was stirred overnight. The resulting solution was diluted with MTBE (200 ml), washed with 1N HCl, 1N NaOH, brine and dried (MgSO₄). The solution was concentrated in vacuo and the residue was washed with MeOH to afford 170 mg (38%) of t-butyl ester as a light yellow solid, which was hydrolyzed by TFA (2 ml) in DCM (10 ml) to afford 90 mg (60%) of a white solid.

Analytical data

¹H NMR (400 MHz, DMSO-d₆) δ 12.75 (s, 1H), 8.85 (d, J=8.9 Hz, 1H), 8.43 (d, J=2.1 Hz, 1H), 8.14 (dd, J=2.1, 8.9 Hz, 1H), 7.95 (dd, J=1.7, 7.9 Hz, 1H), 7.64 (m, 1H), 7.30 (d, J=8.5 Hz, 1H), 7.22 (s, 1H), 7.13 (m, 1H), 4.89 (hept, J=6.0 Hz, 1H), 1.40 (d, J=6.0 Hz, 6 H).

Preparation of 5-{2-[2-(2-Ethoxyethoxy)ethoxy]phenyl}isoxaz le-3-carboxylic acid

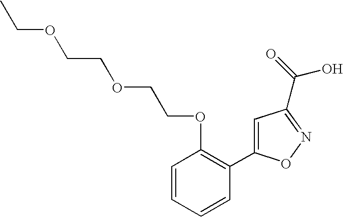

Prepared according to General method A: ethyl-5-(2-hydroxyphenyl)isoxazole-3-carboxylate (500 mg, 2.15 mmol) and 2-(2-ethoxyethoxy)ethanol (288 mg, 2.15 mmol) afforded 410 mg (55%) of ethyl 5-{2-[2-(2-ethoxyethoxy)ethoxy]phenyl}isoxazole-3-carboxylate as a white solid, which was hydrolyzed in MeOH (10 ml) and water (5 ml) by LiOH.H₂O (148 mg, 3.5 mmol) to afford 360 mg (95%) of 5-{2-[2-(2ethoxyethoxy)ethoxy]phenyl}isoxazole-3-carboxylic acid as a yellow solid.

Analytical data

1H NMR (300 MHz, DMSO-d₆) δ 8.02 (dd, J=1.7, 7.8 Hz, 1H), 7.44 (m, 1H), 7.37 (s, 1H), 7.11 (dt, J=0.9, 7.7 Hz, 1H), 7.04 (d, J=8.5 Hz, 1H), 4.32 (m, 2 H), 3.99 (m, 2H), 3.80 (m, 2 H), 3.73 (m, 2H), 3.58 (m, 2H), 1.22 (t, J=7.0 Hz, 3 H).

### Example 7.49: 5-Cyano-2-{[(5-{2-[2-(2-ethoxyethoxy)ethoxylphenyl}isoxazol-3-yl)carbonyl acid

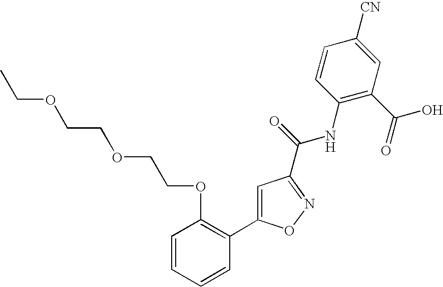

Prepared according to General method B: 5-{2-[2-(2ethoxyethoxy)ethoxy]phenyl}isoxazole-3-carboxylic acid (360 mg, 1.1 mmol) and tertbutyl-2-amino-5-cyano benzoate (284 mg, 1.3 mmol) afforded 170 mg (29%) oftbutyl ester as a white solid, which was hydrolyzed by TFA (2 ml) in DCM (10 ml) to afford 120 nig (79%) of a white solid.

Analytical data

¹H NMR (400 MHz, DMSO-d₆) δ 12.77 (s, 1H), 8.85 (d, J=8.9 Hz, 1H), 8.43 (d, J=2.1 Hz, 1H), 8.14 (dd, J=2.1, 8.7 Hz, 1H), 7.95 (dd, J=1.7, 7.7 Hz, 1H), 7.55 (m, 1H), 7.27 (d, J=8.3 Hz, 1H), 7.22 (s, 1H), 7.15 (t, J=7.9 Hz, 1H), 4.20 (t, J=6.4 Hz, 2 H), 1.85 (pent,J=6.2 Hz, 2 H), 1.60 (pent, J=7.0 Hz, 2 H), 1.35 (m, 4 H), 0.88 (t,J=7.0 Hz,3 H).

Preparation of 5-[2-(2,3-Dihydro-1,4-benzodioxin-2-yhnethoxy)phenyl]isoxazole-3-carboxylic acid

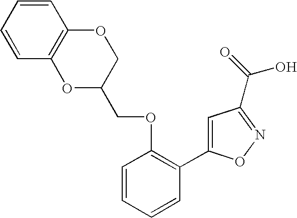

Prepared according to General method A: ethyl-5-(2-hydroxyphenyl)isoxazole-3-carboxylic acid (500 mg, 2.15 mmol) and 2,3-dihydro-1,4-benzodioxin-2-ylmethanol (348 mg, 2.15 mmol) afforded 550 mg (68%) of ethyl 5-[2-(2,3-dihydro-1,4-benzodioxin-2-ylmethoxy)phenyl]isoxazole-3-carboxylate as a yellow solid, which was hydrolyzed in MeOH (10 ml) and water (5 ml) by LiOH.H₂O (148 mg, 3.5 mmol) to afford 470 mg (92%) of 5-[2-(2,3-dihydro-1,4-benzodioxin-2-ylmethoxy)phenyl]isoxazole-3-carboxylic acid as a white solid.

Analytical data

¹H NMR (400 MHz, DMSO-d₆) δ 14.05 (s, 1H), 7.94 (dd, J=1.7, 7.9 Hz, 1H), 7.55 (m, 1H), 7.36 (s, 1H), 7.32 (d, J=8.1 Hz, 1H), 7.18 (m, 1H), 7.01 (m, 1H), 6.92 (m, 1H), 6.86 (m, 2 H), 4.74 (m, 1H), 4.67 (dd, J=3.5, 10.6 Hz, 1H), 4.46 (dd, J=2.5, 11.6 Hz, 1H), 4.33 (dd, J=7.5, 10.6 Hz, 1H), 4.18 (dd, J=6.9, 11.6 Hz, 1H).

### Example 7.50: 5-Cyano-2-[({5-12-(2,3-dihydro-1,4-benzodioxin-2-ylnethoxy)phenyl]isoxazol-3-yl}carbonyl)amino]benzoic acid

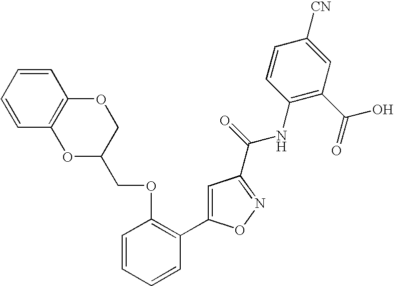

Prepared according to General method B: 5-[2-(2,3-dihydro-1,4-benzodioxin-2-ylmethoxy)phenyl]isoxazole-3-carboxylic acid (470 mg, 1.4 mmol) and tert-butyl-2amino-5-cyano benzoate (267 mg, 1.2 mmol) afforded 360 mg (54%) of t-butyl ester as a white solid, which was hydrolyzed by TFA (2 ml) in DCM (10 ml) to afford 290 mg (89%) of a white solid.

Analytical data

¹H NMR (300 MHz, DMSO-d₆) δ 12.74 (s, 1H), 8.85 (d, J=8.9 Hz, 1H), 8.43 (d, J=2.1 Hz, 1H), 8.13 (dd, J=2.1, 8.9 Hz, 1H), 7.98 (dd, J=1.7, 7.9 Hz, 1H), 7.57 (m, 1H), 7.48 (s, 1H), 7.34 (d, J=8.3 Hz, 1H), 7.20 (t, J=7.7 Hz, 1H), 7.04 (m, 1 H), 6.90 (m, 3 H), 4.76 (m, 1H), 4.59 (dd, J=3.8, 10.7 Hz, 1H), 4.47 (dd, J=2.3, 11.5 Hz, 1H), 4.35 (dd, J=7.4, 10.6 Hz, 1H), 4.20 (dd, J=6.6, 11.3 Hz, 1H).

Preparation of 5-[2-(Hexyloxy)phenyl]isoxazole-3-carboxylic acid

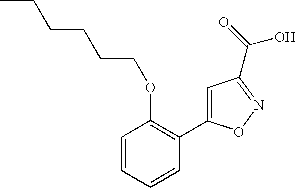

General method C: ethyl-5-(2-hydroxylphenyl)isoxazole-3-carboxylic acid (500 mg, 1.3 mmol), K₂CO₃ (455 mg, 4.29 mmol) and n—Iodohexane were place in acetonitrile (20 ml). The mixture was stirred over night. The resulting solution was loaded on silica gel and purified by flashing chromatography ((Heptane/EtOAc=1000/10, 1000/20) to afford 590 mg (87%) of ethyl 5-[2-(hexyloxy)phenyl]isoxazole-3-carboxylate as a yellow solid, which was hydrolyzed in MeOH (10 ml) and water (5 ml) by LiOH.H₂O (137 mg, 3.3 ummol) to afford 528 mg (98%) of 5-[2-(hexyloxy)phenyl]isoxazole-3-carboxylic acid as a yellow solid.

Analytical data

¹H NMR (400 MHz, DMSO-d₆) δ 7.91 (dd, J=1.7, 7.9 Hz, 1H), 7.52 (m, 1H), 7.24 (d,J=8.1 Hz, 1H),7.12(m, 1H),7.08(s, 1H),4.17(t,J=6.2 Hz,2H), 1.82(m,2 H), 1.48 (m, 2 H), 1.32 (m, 4 H), 0.87 (t, J=7.1 Hz, 3 H).

### Example 7.51: 5-Cyano-2-[({5-[2-(hexyloxy)phenyl]isoxazol-3-yl}carbonyl)aminojbenzoic acid

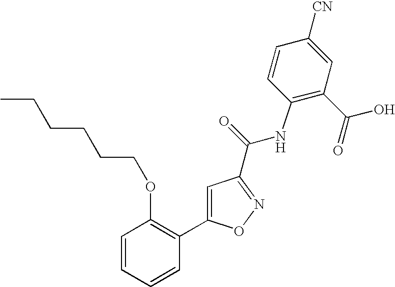

Prepared according to General method B: 5-[2-(hexyloxy)phenyl]isoxazole-3-carboxylic acid (528 mg, 1.8 mmol) and tert-butyl-2-amino-5-cyano benzoate (362 mg, 1.7 mmol) afforded 430 mg (53%) of t-butyl ester as a white solid, which was hydrolyzed by TFA (2 ml) in DCM (10 ml) to afford 290 mg (76%) of a white solid.

Analytical data

¹H NMR (400 MHz, DMSO-d₆) δ 12.77 (s, 1H), 8.85 (d, J=8.9 Hz, 1H), 8.43 (d, J=2.1 Hz, 1H), 8.14 (dd, J=2.1, 8.7 Hz, 1H), 7.95 (dd, J=1.7, 7.7 Hz, 1), 7.55 (m, 1H), 7.27 (d, J=8.3 Hz, 1H), 7.22 (s, 1H), 7.15 (t, J=7.9 Hz, 1H), 4.20 (t, J =6.4 Hz, 2 H), 1.85 (pent, J=6.2 Hz, 2 H), 1.60 (pent, J=7.0 Hz, 2 H), 1.35 (m, 4 H), 0.88 (t,J=7.0 Hz, 3 H).

Preparation of 5-12-(Allyloxy)phenyl]isoxazole-3-carboxylic acid

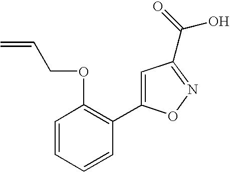

Prepared according to General method C: ethyl-5-(2-hydroxyphenyl)isoxazole-3-carboxylic acid (500 mg, 1.3 mmol) and allyl bromide (286 mg, 2.4 mmol) afforded 540 mg (92%) of 5-[2-(allyloxy)phenyl]isoxazole-3-carboxylic acid as a yellow solid, which was hydrolyzed in MeOH (10 ml) and water (5 ml) by LiOH.H₂O (137 mg, 3.3 mmol) to afford 480 mg (99%) of 5-[2-(allyloxy)phenyl]isoxazole-3-carboxylic acid as a yellow solid.

Analytical data

¹H NMR (400 MHz, DMSO-d₆) δ 14.12 (s, 1H), 7.92 (dd, J=1.7, 7.7 Hz, 1H), 7.53 (m, 1H), 7.26 (d, J=8.1 Hz, 1H), 7.14 (rm, 1H), 7.10 (s, 1H), 6.15 (m, 1H (dd, J=1.7, 17.2 Hz, 1H), 5.35 (dd, J=1.7, 10.6 Hz, 1H), 4.90 (dt, J=1.2, 5.4 Hz, 2 H)

### Example 7.52: 2-[({5-[2-(Allyloxy)phenyl]isoxazol-3-yl}carbonyl)aminol-5-cyanobenzoic acid

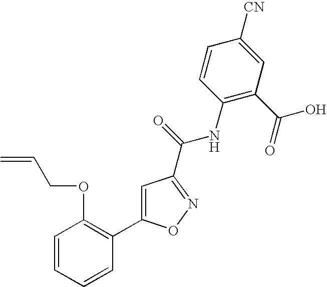

Prepared according to General method B: 5-[2-(allyloxy)phenyl]isoxazole-3-carboxylic acid (480 mg, 2.0 mmol) and tert-butyl-2-amino-5-cyano benzoate (388 mg, 1.8 mmol) afforded 330 mg (42%) of t-butyl ester as a white solid, 100 mg of which was hydrolyzed by TFA (2 ml) in DCM (10 ml) to afford 60 mg (69%) of a white solid.

Analytical data

¹H NMR (400 MHz, DMSO-d₆) δ 12.76 (s, 1H), 8.85 (d, J=8.7 Hz, 1H), 8.33 (d, J=1.9 Hz, 1H), 8.14 (dd, J=2.1, 8.7 Hz, 1H), 7.96 (dd, J=1.7, 7.9 Hz, 1H), 7.65 (m, 1H), 7.29 (d, J=8.5 Hz, 1H), 7.25 (s, 1H), 7.17 (t, J=7.7 Hz, 1H), 6.17 (m, 1 H), 5.48 (dd, J=1.5, 17.2 Hz, 1H), 5.37 (dd, J=1.5, 10.6 Hz, 1H), 4.82 (d , J=5.5 Hz, 2 H).

Preparation of 5-(2-Ethoxyphenyl)isoxazole-3-carboxylic acid

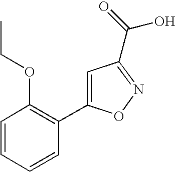

Prepared according to General method A: ethyl-5-(2-hydroxyphenyl)isoxazole-3-carboxylic acid (500 mg, 2.15 mmol) and tetrahydro-2H-pyran-2-ylmethanol (249 mg, 2.15 mmol) afforded 110 mg (25%) of ethyl 5-(2-ethoxyphenyl)isoxazole-3-carboxylate as a white solid, which (plus another 110 mg from 36831-jl-34) was hydrolyzed in MeOH (10 ml) and water (5 ml) by LiOH.H₂O (148 mg, 3.5 mmol) to afford 200 mg (100%) of 5-(2-ethoxyphenyl)isoxazole-3-carboxylic acid as a white solid.

Analytical data

¹H NMR (300 MHz, DMSO-d₆) δ 7.92 (dd, J=1.7, 7.9 Hz, 1), 7.52 (m, 1H), 7.24 (d, J=8.3 Hz, 1H), 7.12 (t, J=7.7 Hz, 1H), 7.08 (s, 1H), 4.24 (tetra, J=7.0 Hz, 2 H), 1.45 (t, J=7.0 Hz, 3 H).

### Example 7.53: 5-Cyano-2-({[5-(2-ethoxyphenyl)isoxazol-3-yl]carbonyl}amino)benzoic acid

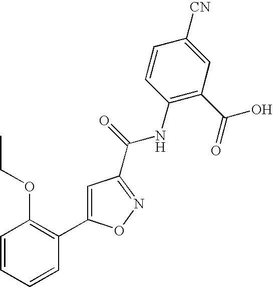

Prepared according to General method B: 5-(2-ethoxyphenyl)isoxazole-3-carboxylic acid (200 mg, 0.86 mmol) and tert-butyl-2-amino-5-cyano benzoate (170 mg, 0.78 mmol) afforded 130 mg (38%) of t-butyl ester as a white solid, which was hydrolyzed by TFA (2 ml) in DCM (10 ml) to afford 98 mg (87%) of a white solid.

Analytical data

¹H NMR (300 MHz, DMSO-d₆) δ 12.77 (s, 1H), 8.85 (d, J=8.9 Hz, 1H), 8.43 (d, J=1.9 Hz, 1H), 8.14 (dd, J=2.1, 8.9 Hz, 1H), 7.96 (dd, J=1.5, 7.9 Hz, 1H), 7.55 (m, 1H), 7.26 (d, J=8.3 Hz, 1H), 7.23 (s, 1H), 7.15 (t, J=7.7 Hz, 1H), 4.27 (tetra, J=7.0 Hz, 2 H), 1.48 (t, J=7.0 Hz, 3 H).

Preparation of 5-[2-(tetrahydro-2H-pyran-2-ylmethoxy)phenyl]isoxazole-3-carboxylic acid

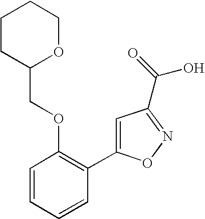

General method D: ethyl-5-(2-hydroxyphenyl)isoxazole-3-carboxylic acid (500 mg, 2.15 mmol) and triphenylphosphine (562 mg, 2.15 mmol) were dissolved in THF (5 ml). tetrahydro-2H-pyran-2-ylmethanol (249 mg, 2.15 mmmol) was added, followed by the addition of Di-tert-butylazocarboxylate (DIAD) (495 mg, 2.15 mmol). The mixture was shaken over mght. The resulting solution was loaded on silica gel and purified by flashing chromatography ((Heptane/EtOAc=1000/10, 1000/20) to afford 380 mg (54%) of ethyl 5-[2-(tetrahydro-2H-pyran-2-ylmethoxy)phenyl]isoxazole-3-carboxylate as a yellow solid, which was hydrolyzed in MeOH (10 ml) and water (5 ml) by LiOH.H₂O (137 mg, 3.3 mmol) to afford 360 mg (100%) of 5-[2-(tetrahydro-2H-pyran-2-ylmethoxy)phenyl]isoxazole-3-carboxylic acid as a yellow oil.

Analytical data

¹H NMR (300 MHz, DMSO-d₆) δ 8.02 (dd, J=1.5, 7.7 Hz, 1H), 7.53 (s, 1H), 7.44 (m, 1H), 7.10 (t, J=7.7 Hz, 1H), 7.02 (d, J=8.5 Hz, 1H), 4.14 (m, 3 H), 3.86 (m, 1H), 3.61 (m, 1H), 1.97 (m, 1H), 1.65 (m, 5 H).

### Example 7.54: 5-Cyano-2-[({5-[2-(tetrahydro-2H-pyran-2-ylmethoxy)phenyl]isoxazol-3-yl}carb acid

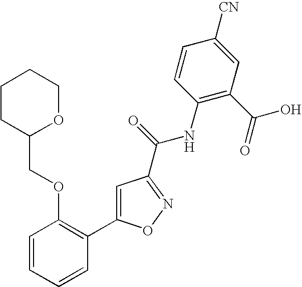

Prepared according to General method B: 5-[2-(tetrahydro-2H-pyran-2-ylmethoxy)phenyl]isoxazole-3-carboxylic acid (360 mg, 1.2 mmol) and tert-butyl-2amino-5-cyano benzoate (235 mg, 1.1 mmol) afforded 270 mg (50%) of t-butyl ester as a white solid, which was hydrolyzed by TFA (2 ml) in DCM (10 ml) to afford 200 mg (83%) of a white solid.

Analytical data

¹H NMR (400 MHz, DMSO-d₆) δ 12.75 (s, 1H), 8.87 (d, J=8.9 Hz, 1H), 8.43 (d, J=2.1 Hz, 1H), 8.14 (dd, J=2.1,8.7 Hz, 1H), 7.96 (dd, J=1.7,7.9 Hz, 1H), 7.65 (s, 1H), 7.54 (m, 1H), 7.25 (d, J=8.3 Hz, 1H), 7.16 (m, 1H), 4.21 (dd, J=3.1, 10.2 Hz, 1H), 4.11 (dd, J=6.0, 10.2 Hz, 1H), 4.03 (d, J=11.0 Hz, 1H), 3.76 (m, 1 H), 3.50 (m, 1H), 1.86 (m, 1H), 1.66 (d, J=11.2 Hz, 1H), 1.67 (m, 4 H).

Preparation of 5-[2-(3-Phenoxypropoxy)phenyl]isoxazole-3-carboxylic acid

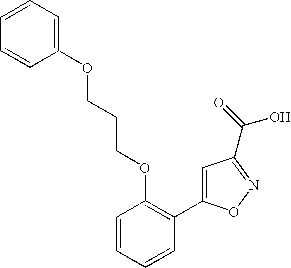

Prepared according to General method D: ethyl-5-(2-hydroxyphenyl)isoxazole-3-carboxylic acid (500 mg, 2.15 mmol) and 3-phenoxypropan-1-ol (327 mg, 2.15 mmol) afforded 660 mg (88%) of ethyl 5-[2-(3-phenoxypropoxy)phenyl]isoxazole-3-carboxylate as a yellow solid, which was hydrolyzed in MeOH (10 ml) and water (5 ml) by LiOH.H₂O (300 mg, 7.1 mmol) to afford 540 mg (88%) of 5-[2-(3phenoxypropoxy)pheny acid as a yellow solid.

Analytical data

¹H NMR (300 MHz, CD₃OD) 5 7.94 (dd, J=1.9, 8.3 Hz, 1H), 7.43 (m, 1H), 7.24 (m, 2 H), 7.15 (s, 1H), 7.08 (d,J=7.9 Hz, 2 H), 6.91 (m, 3 H), 4.35 (t,J=6.2 Hz, 2 H), 4.19 (t, J=5.8 Hz, 2 H), 3.88 (pent, J=6.0 Hz, 2 H).

### Example 7.55: 5-Cyano-2-[({5-[2-(3-phenoxypropoxy)phenyl]isoxazol-3-yl}carbonyl)amino]benzoic acid

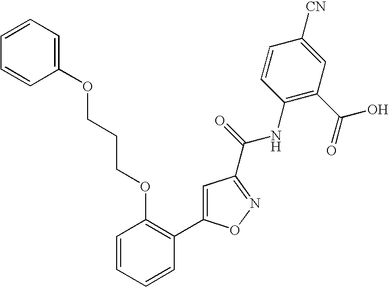

Prepared according to General method B: 5-[2-(3-phenoxypropoxy)phenyl]isoxazole-3-carboxylic acid (540 mg, 1.7 mmol) and tert-butyl-2-amino-5-cyano benzoate (331 mg, 1.5 mmol) afforded 500 mg (63%) of t-butyl ester as a yellow solid, which was hydrolyzed by TFA (2 ml) in DCM (10 ml) to afford 410 mg (89%) of a white solid.

Analytical data

¹H NMR (300 MHz, DMSO-d₆) δ 12.73 (s, 1H), 8.85 (d, J=8.9 Hz, 1H), 8.43 (d, J=2.1 Hz, 1H), 8.14 (dd, J=2.1, 8.9 Hz, 1H), 7.95 (dd, J=1.5, 7.9 Hz, 1H), 7.55 (m, 1H), 7.32 (d, J=8.9 Hz, 1H), 7.26 (m, J=3 Hz, H), 7.16 (t, J=7.5 Hz, 1H), 6.94 (m, 3 H), 4.38 (t, J=6.0 Hz, 2 H), 4.20 (t, J=6.2 Hz, 2 H), 2.82 (pent, J=6.2 Hz, 2 H).

Preparation of 5-(2-{1(1S)-1-phenylethyl]oxy}phenyl)isoxazole-3-carboxylic acid

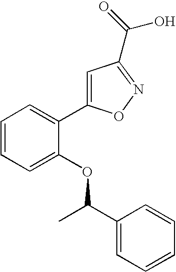

Prepared according to General method D: ethyl-5-(2-hydroxyphenyl)isoxazole-3-carboxylage acid (500 mg, 2.15 mmol) and 1(1 S)-1-phenylethanol (262 mg, 2.15 mmol) afforded 440 mg (57%) of ethyl 5-(2-{[(S)—Iphenylethyl]oxy}phenyl)isoxazole-3-carboxylate as a green oil, which was hydrolyzed in MeOH (10 ml) and water (5 ml) by LiOH.H₂O (200 mg, 4.8 mmol) to afford 380 mg (100%) of 5-(2-{[(1S)-1-phenylethyl]oxy}phenyl)isoxazole-3-carboxylic acid as a yellow solid.

Analytical data

¹H NMR (300 MHz, DMSO-d₆) δ 7.90 (dd, J=1.5, 7.7 Hz, 1H), 7.37 (m, 5 H), 7.28 (m, 1H), 7.23 (s, 1H), 7.06 (m, 2 H), 5.78 (tetra, J=6.4 Hz, 1H), 1.68 (d, J=6.2 Hz, 3 H);

### Example 7.56: 5-Cyano-2-({[5-(2-hydroxyphenyl)isoxazol-3-yl]carbonyl}amino)benzoic acid

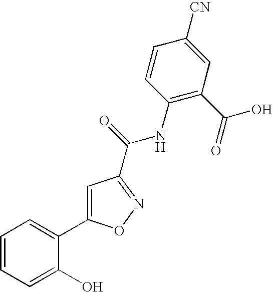

Prepared according to General method B: 5-(2-{[(I S)-iphenylethyl]oxy}phenyl)isoxazole-3-carboxylic acid (380 mg, 1.2 mmol) and tertbutyl-2-amino-5-cyano benzoate (244 mg, 1.1 Immol) afforded 45 mg (8%) of tertbutyl

5-cyano-2-({[5-(2-{[(1S)-i -phenylethyl]oxy}phenyl)isoxazol-3-yl]carbonyl}amino)benzoate as a white solid, which was hydrolyzed by TFA (2 ml) in DCM (10 ml) to afford 18 mg (58%) of a white solid.

Analytical data

¹H NMR (400 MHz, DMSO-d₆) δ 12.70 (s, 1H), 10.87 (s, 1H), 8.86 (d, J=8.7 Hz, 1H), 8.44 (d, J=2.1 Hz, 1H), 8.15 (dd, J=2.1, 8.9 Hz, 1), 7.87 (dd, J=1.7, 7.9 Hz, 1H), 7.89 (m, 1H), 7.26 (s, 1H), 7.09 (d, J=7.7 Hz, 1H), 7.01 (t, J=7.0 Hz, 1 H).

Preparation of 5-[2-(Cyclobutyloxy)phenyl]isoxazole-3-carboxylic acid

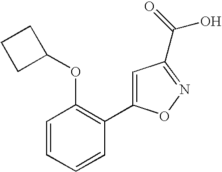

Prepared according to General method D: ethyl-5-(2-hydroxyphenyl)isoxazole-3-carboxylic acid (500 mg, 2.15 mmol) and cyclobutanol (155 mg, 2.15 mmol) afforded 260 mg (42%) of ethyl 5-[2-(cyclobutyloxy)phenyl]isoxazole-3-carboxylate as a yellow solid, which was hydrolyzed in MeOH (10 ml) and water (5 ml) by LiOH.H₂O (300 mg, 7.1 mmol) to afford 200 mg (82%) of 5-[2-(cyclobutyloxy)phenyl]isoxazole-3-carboxylic acid as a yellow solid.

Analytical data

¹H NMR (400 MHz, DMSO-d₆) δ 7.91 (dd, J=1.5, 7.7 Hz, 1H), 7.50 (m, 1H), 7.10 (s, 1H), 7.09 (m, 2 H), 4.90 (m, 1H), 2.54 (mn, 1H), 2.14 (m, 2 H), 1.81 (m, 1H), 1.71 (m, 1H), 1.37 (d, J=6.0 Hz, 1H);

### Example 7.57: 5-Cyano-2- [(5-[2-(cyclobutyloxy)phenyl]isoxazol-3-yl}carbonyl)amino]benzoic acid

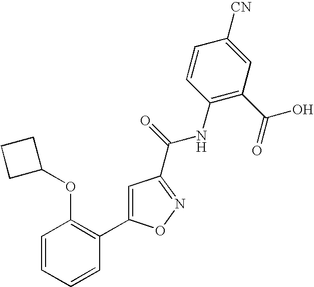

Prepared according to General method B: 5-[2-(cyclobutyloxy)phenyl]isoxazole-3-carboxylic acid (200 mg, 0.77 mmol) and tert-butyl-2-amino-5-cyano benzoate(154 mg, 0.7 mmol) afforded 140 mg (44%) of t-butyl ester as a white solid, which was hydrolyzed by TFA (2 ml) in DCM (10 ml) to afford 67 mg (55%) of a white solid.

Analytical data

¹H NMR (400 MHz, DMSO-d₆) δ 12.75 (s, 1H), 8.85 (d, J=8.9 Hz, 1H), 8.43 (d, J=1.9 Hz, 1H), 8.15 (dd, J=2.1, 8.9 Hz, 1H), 7.95 (dd, J 1.7, 7.7 Hz, 1H), 7.52 (m, 1H), 7.24 (s, 1H), 7.14 (t, J=7.7 Hz, 1H), 7.10 (d, J=8.1 Hz, 1H), 4.92 (pent, J=7.1 Hz, 1H), 2.55 (m, 1H), 2.18 (m, 2 H), 1.87 (tetra, J=10.4 Hz, 1H), 1.71 (m, 1H), 1.40 (d, J=6.0 Hz, 1H).

Preparation of 5-[2-(Tetrahydrofuran-3-yloxy)phenyl]isoxazole-3-carboxylic acid

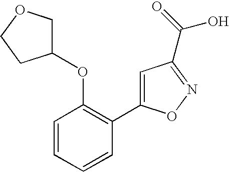

Prepared according to General method D: ethyl-5-(2-hydroxyphenyl)isoxazole-3-carboxylic acid (500 mg, 2.15 mmmol) and tetrahydrofuran-3-ol (155 mg, 2.15 mmol) afforded 1000 mg (100%) of ethyl 5-[2-(tetrahydrofuran-3-yloxy)phenyl]isoxazole-3-carboxylate as a yellow solid, which was hydrolyzed in MeOH (10 ml) and water (5 ml) by LiOH.H₂O (300 mg, 7.1 mmol) to afford 810 mg (89%) of crude 5-[2(tetrahydrofuran-3-yloxy)phenyl]isoxazole-3-carboxylic acid as a yellow solid.

### Example 7.58: 5-Cyano-2-[(({-[2-(tetrahydrofuran-3-yloxy)phenyl]isoxazol-3-yl}carbonyl)amino]benzoic acid

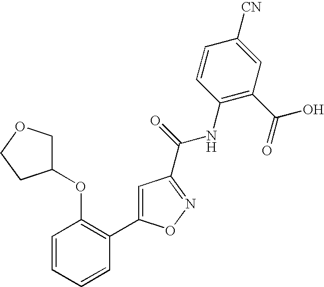

Prepared according to General method B: 5-[2-(tetrahydrofuran-3-yloxy)phenyl]isoxazole-3-carboxylic acid (810 mg, 3.0 mmol) and tert-butyl-2-amimo

5-cyano benzoate (584 mg, 2.7 mmol) afforded 440 mg (35%) of t-butyl ester as a white solid, which was hydrolyzed by TFA (2 ml) in DCM (10 ml) to afford 410 mg (100%) of a white solid.

Analytical data

¹H NMR (400 MHz, DMSO-d₆) δ 12.71 (s, 1H), 8.84 (d, J=8.7 Hz, 1H), 8.42 (d, J=2.1 Hz, 1H), 8.14 (dd, J=2.1, 8.7 Hz, 1H), 7.96 (dd, J=1.7, 7.7 Hz, 1H), 7.55 (m, 1H), 7.28 (d, J=8.3 Hz, 1H), 7.18 (s, 1H), 7.17 (t, J=7.3 Hz, 1H), 5.82 (m, 1 H), 3.94 (m, 3 H), 3.82 (m, 1H), 2.33 (m, 1H), 2.07 (m, 1H).

Preparation of 5-12-(Tetrahydro-2H-pyran-4-yloxy)phenyl]isoxazole-3-carboxylic acid

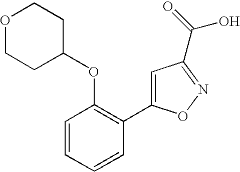

Prepared according to General method D: ethyl-5-(2-hydroxyphenyl)isoxazole-3-carboxylic acid (500 mg, 2.15 mmol) and tetrahydro-2H-pyran-4-ol (219 mg, 2.15 mmol) afforded 1000 mg (100%) of ethyl 5-[2-(tetrahydro-2H-pyran-4-yloxy)phenyl]isoxazole-3-carboxylic as a white solid, which was hydrolyzed in MeOH (10 ml) and water (5 ml) by LiOH.H₂O (300 mg, 7.1 mmol) to afford 780 mg (85%) of 5-[2-(tetrahydro-2H-pyran-4-yloxy)phenyl]isoxazole-3-carboxylic acid as a white solid.

Analytical data

¹H NMR (400 MHz, DMSO-d₆) δ 8.88 (s, 1H), 7.92 (dd, J=1.7, 7.9 Hz, 1H), 7.51 (m, 1H), 7.35 (d, J=8.3 Hz, 1H), 7.12 (t, J=7.3 Hz, 1H), 7.10 (s, 1H), 4.77 (m, 1 H), 3.86 (m, 2 H), 3.54 (m, 2 H), 2.07 (m, 2 H), 1.68 (m, 2 H).

### Example 7.59: 5-Cyano-2-[({5-[2-(tetrahydro-2H-pyran-4-yloxy)phenyl]isoxazol-3-yl}carbonyl)amino]benzoic acid

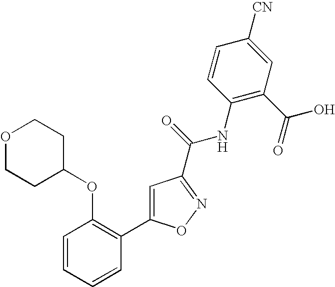

Prepared according to General method B: 5-[2-(tetrahydro-2H-pyran-4-yloxy)phenyl]isoxazole-3-carboxylic acid (780 mg, 2.7 mmol) and tert-butyl-2-amino-5-cyano benzoate (535 mg, 2.5 mmol) afforded 950 mg (79%) of t-butyl ester as a brown solid, which was hydrolyzed by TFA (2 ml) in DCM (10 ml) to afford 350 mg (42%) of a white solid.

Analytical data

¹H NMR (300 MHz, DMSO-d₆) δ 12.75 (s, 1), 8.86 (d, J=8.9 Hz, 1H), 8.44 (d, J=2.1 Hz, 1H), 8.15(dd,J=2.1, 8.9 Hz, 1H), 7.96(dd,J=1.5, 7.7 Hz, 1H),7.54 (m, 1H), 7.38 (d, J=8.3 Hz, 1H), 7.24 (s, 1H), 7.15 (t, J=7.5 Hz, 1H), 4.88 (hept, J=4.2 Hz, 1H), 3.90 (t, J=4.1 Hz, 1H), 3.86 (t, J=4.5 Hz, 1H), 3.55 (m, 2 H), 2.09 (m, 2 H), 1.72 (m, 2 H).

Preparation of 5-(2-{[(1S,2R)-2-methylcyclopentyl]oxy}phenyl)isoxazole-3-carboxylic acid

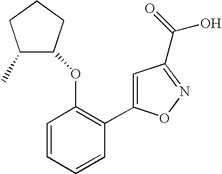

Prepared according to General method D: ethyl-5-(2-hydroxyphenyl)isoxazole-3-carboxylic acid (500 mg, 2.15 mmol) and (I R,2R)-2-methylcyclopentanol (215 mg, 2.15 mmol) afforded 430 mg (64%) of ethyl 5-(2-{[(1S,2R)-2-methylcyclopentyl]oxy}phenyl)isoxazole-3-carboxylate as a yellow oil, which was hydrolyzed in MeOH (10 ml) and water (5 ml) by LiOH.H₂O (300 mg, 7.1 mmol) to afford 360 mg (91%) of 5-(2-{[(IS,2R)-2-methylcyclopentyl]oxy}phenyl)isoxazole-3-carbo acid as a white solid.

Analytical data

¹H NMR (400 MHz, DMSO-d₆) δ 7.90 (dd, J=1.7, 7.9 Hz, 1H), 7.60 (m, 1H), 7.27 (d, J=8.3 Hz, 1H), 7.10 (m, 1H), 7.08 (s, 1H), 4.92 (dt, J=1.5, 4.5 Hz, 1H), 2.19 (m, 1H), 2.03 (m, 1H), 1.90 (m, 1H), 1.73 (m, 2 H), 1.62 (m, 1H), 1.60 (m, 1H), 1.05(d,J=6.8 Hz,3 H).

### Example 7.60: 5-Cyano-2-({[5-(2-{[(1S,2R)-2-methylcyclopentyl]oxy}phenyl)isoxazol-3-yl]carbonyl}amino)benzoic acid

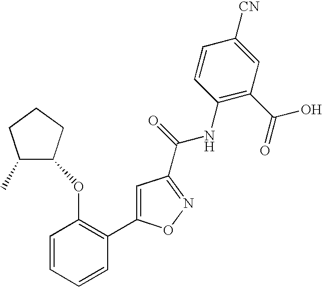

Prepared according to General method B: 5-(2-{[(1S,2R)-2-methylcyclopentyl]oxy}phenyl)isoxazole-3-carboxylic acid (360 mg, 1.3 mmol) and tert-butyl-2-amino-5-cyano benzoate(249 mg, 1.1 mmol) afforded 270 mg (49%) of t-butyl ester as a white solid, which was hydrolyzed by TFA (2 ml) in DCM (10 ml) to afford 250 mg (42%) of a white solid.

Analytical data

¹H NMR (400 MHz, DMSO-d₆) δ 12.77 (s, 1H), 8.86 (d, J=8.9 Hz, 1H), 8.43 (d, J=8.1 Hz, 1H),8.13(dd,J=2.1,8.7 Hz, 1H),7.95(dd,J=1.7,7.9 Hz, 1H),7.53 (m, 1H), 7.30 (d, J=8.3 Hz, 1H), 7.22 (s, 1H), 7.13 (m, 1H), 4.95 (m, 1H), 2.21 (m, 1H), 2.05 (m, 1H), 1.93 (m, 1H), 1.76 (m, 2 H), 1.65 (m, 1H), 1.54 (m, 1H), 1.07(d,J=6.8 Hz, 3 H).

Preparation of 5—I2-(2-Methoxy-1-methylethoxy)phenyl]isoxazole-3-carboxylic acid

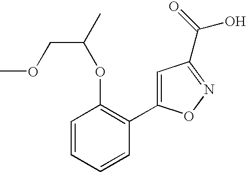

Prepared according to General method D: ethyl-5-(2-hydroxyphenyl)isoxazole-3-carboxylate acid (500 mg, 2.15 mmol) and 1-methoxypropan-2-ol (194 mg, 2.15 mmol) afforded 570 mg (87%) of ethyl 5-[2-(2-methoxy-1-methylethoxy)phenyl]isoxazole-3-carboxyla as a yellow oil, which was hydrolyzed in MeOH (10 ml) and water (5 ml) by LiOH.H₂O (300 mg, 7.1 mmol) to afford 430 mg (83%) of 5-[2-(2-methoxy-1-methylethoxy)phenyl]isoxazole-3-carboxylic acid as a white solid.

Analytical data

¹H NMR (300 MHz, DMSO-d₆) δ 7.91 (dd, J=1.7, 7.9 Hz, 1H), 7.50 (m, 1H), 7.30 (d, J=8.3 Hz, 1H), 7.21 (s, 1H), 7.11 (m, 1H), 4.86 (m, 1H), 3.59 (d,J=4.5 Hz, 2 H), 3.32 (s, 3 H), 1.31 (d, J=6.2 Hz, 3 H).

### Example 7.61: 5-Cyano-2-[({5-[2-(2-methoxy-1-methylethoxy)phenyl]isoxazol-3-yl}carbonyl)amino]benzoic acid

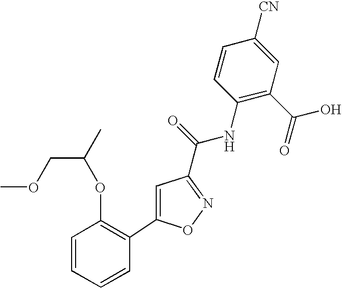

Prepared according to General method B: 5-[2-(2-methoxy-1-methylethoxy)phenyl]isoxazole-3-carboxylic acid (430 mg, 1.6 mmol) and tert-butyl-2amino-5-cyano benzoate (308 mg, 1.4 mmol) afforded 360 mg (54%) of t-butyl ester as a white solid, which was hydrolyzed by TFA (2 ml) in DCM (10 ml) to afford 160 mg (50%) of a white solid.

Analytical data

¹H NMR (400 MHz, DMSO-d₆) δ 12.75 (s, 1H), 8.86 (d, J=8.9 Hz, 1H), 8.43 (d, J=2.1 Hz, 1H), 8.14 (dd, J=2.1, 8.7 Hz, 1), 7.96 (dd, J=1.7, 7.9 Hz, 1H), 7.53 (m, 1H), 7.36 (s, 1H), 7.33 (d, J=8.5 Hz, 1H), 7.14 (t, J=7.1 Hz, 1H), 4.88 (m, 1 H),3.63(d,J=4.4 Hz,2H),3.35(s,3H), 1.34(d,J=6.3 Hz,3H).

Preparation of 5-12-(1-Methylbutoxy)phenyl]isoxazole-3-carboxylic acid

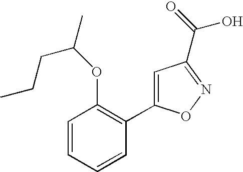

Prepared according to General method D: ethyl-5-(2-hydroxyphenyl)isoxazole-3-carboxylate acid (500 mg, 2.15 mmol) and pentan-2-ol (189 mg, 2.15 mmol) afforded 530 mg (82%) of ethyl 5-[2-(1-methylbutoxy)phenyl]isoxazole-3-carboxylate as a yellow oil, which was hydrolyzed in MeOH (10 ml) and water (5 ml) by LiOH.H₂O (300 mg, 7.1 mmol) to afford 450 mg (93%) of 5-[2-(1-methylbutoxy)phenyl]isoxazole-3-carboxylic acid as a colomless oil.

Analytical data

¹H NMR (300 MHz, DMSO-d₆) δ 7.91 (dd, J=1.7, 7.9 Hz, 1H), 7.50 (m, 1H), 7.27 (d, J=8.3 Hz, 1H), 7.10 (m, 1H), 7.06 (s, 1H), 4.73 (hex, J=6.0 Hz, 1H), 1.69 (m, 2 H), 1.41 (m,2 H), 1.32 (d, J=6.0 Hz, 3 H), 0.91 (t, J=7.4 Hz, 3 H).

### Example 7.62: 5-Cyano-2-[({5-[2-(1-methylbutoxy)phenyl]isoxazol-3-yl}carbonyl)amino]benzoic acid

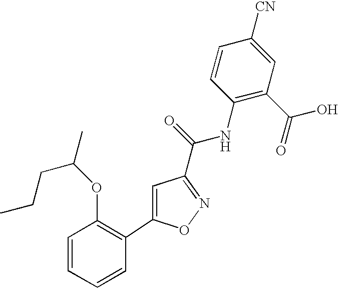

Prepared according to General method B: 5-[2-(1-methylbutoxy)phenyl]isoxazole-3-carboxylic acid (450 mg, 1.6 mmol) and tert-butyl-2-amino-5-cyano benzoate (324 mg, 1.5 mmol) afforded 320 mg (45%) of t-butyl ester as a white solid, which was hydrolyzed by TFA (2 ml) in DCM (10 ml) to afford 170 mg (60%) of a white solid.

Analytical data

¹H NMR (400 MHz, DMSO-d₆) δ 12.79 (s, J=1Hz, H), 8.86 (d, J=8.7 Hz, 1H), 8.44 (d, J=2.1 Hz, 1H), 8.14 (dd, J=2.1, 8.7 Hz, 1H), 7.96 (dd, J=1.7, 7.7 Hz, 1 H), 7.64 (m, 1H), 7.30 (d, J=8.5 Hz, 1H), 7.21 (s, 1H), 7.12 (t, J=7. 3 Hz, 1H), 4.76 (hex, J=6.2 Hz, 1H), 1.79 (m, 1H), 1.68 (m, 1H), 1.45 (m, 2 H), 1.34 (d, J=6.0 Hz, 3 H), 0.92 (t, J=7.3 Hz, 3 H).

Preparation of 5-[2-(Cyclobutylmethoxy)phenyl]isoxazole-3-carboxylic acid

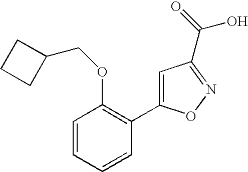

Prepared according to General method D: ethyl-5-(2-hydroxyphenyl)isoxazole-3-carboxylate acid (500 mg, 2.15 mmol) and cyclobutylmethanol (185 mg, 2.15 mmol) afforded 420 mg (65%) of ethyl 5-[2-(cyclobutylmethoxy)phenyl]isoxazole-3-carboxylate as a yellow oil, which was hydrolyzed in MeOH (10 ml) and water (5 ml) by LiOH.H₂O (300 mg, 7.1 mmol) to afford 360 mg (94%) of 5-[2(cyclobutylmethoxy)phenyl]isox acid as a white solid.

Analytical data

¹H NMR (400 MHz, DMSO-d₆) δ 7.92 (dd, J=1.7, 7.9 Hz, 1H), 7.52 (m, 1H), 7.24 (d, J=8.3 Hz, 1), 7.13 (t, J=7.7 Hz, 1), 7.07 (s, 1), 4.14 (d, J=6.6 Hz, 2 H) 2.86(m, 1H),2.12((m,2H), 1.95((m,4H).

### Example 7.63: 5-Cyano-2-[({5-[2-(cyclobutylmethoxy)phenyl]isoxazol-3-yl}carbonyl)amino]benzoic acid

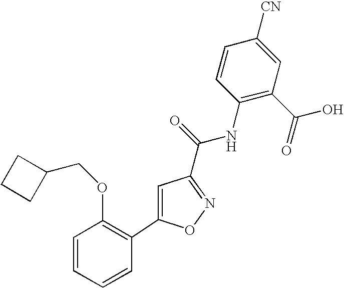

Prepared according to General method B: 5-[2-(cyclobutylmethoxy)phenyl]isoxazole-3-carboxylic acid (360 mg, 1.3 mmol) and tert-butyl-2-amino-5-cyano benzoate (261 mg, 1.2 mmol) afforded 470 mg (83%) of t-butyl ester as a white solid, which was hydrolyzed by TFA (2 ml) in DCM (10 ml) to afford 340 mg (82%) of a white solid.

Analytical data

¹H NMR (400 MHz, DMSO-d₆) δ 12.73 (s, 1H), 8.85 (d, J=8.7 Hz, 1H), 8.42 (d, J=2.1 Hz, 1H), 8.12 (dd, J=2.1, 8.7 Hz, 1H), 7.95 (dd, J=1.7, 7.7 Hz, 1H), 7.54 (m, 1H), 7.26 (d, J=8.3 Hz, 1H), 7.20 (s, 1H), 7.15 (m, 1H), 4.16 (d, J=6.6 Hz, 2 H), 2.89 (m, 1H), 2.14 (m, 2 H), 1.97 (m, 4 H).

Preparation of 5-[2-(Cyclohex-2-en-1-ylmethoxy)phenyl]isoxazole-3-carboxylic acid

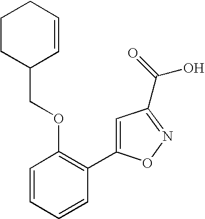

Prepared according to General method D: ethyl-5-(2-hydroxyphenyl)isoxazole-3-carboxylate (500 mg, 2.15 mmol) and cyclobutylmethanol (241 mg, 2.15 mmol) afforded 390 mg (56%) of ethyl 5-[2-(cyclohex-2-en-1-ylmethoxy)phenyl]isoxazole-3-carboxylate as a white soild, which was hydrolyzed in MeOH (10 ml) and water (5 ml) by LiOH.H₂O (300 mg, 7.1 mmol) to afford 370 mg (100%) of 5-[2-(cyclohex-2-en-1-ylmeth acid as a white solid.

Analytical data

¹H NMR (400 MHz, DMSO-d₆) δ 7.92 (dd, J=1.7, 7.9 Hz, 1H), 7.52 (m, 1H), 7.27 (d, J=8.3 Hz, 1H), 7.13 (t, J=7.5 Hz, 1H), 7.08 (s, 1H), 5.71 (s, 2 H), 4.09 (d, J=6.2 Hz, 2 H), 2.19 (m, 2 H), 2.08 (m, 2 H), 1.89 (m, 2 H), 1.41 (1H).

### Example 7.64: 5-Cyano-2-[({5-[2-(cyclobutylmethoxy)phenyl]isoxazol-3-yl}carbonyl)amino]benzoic acid

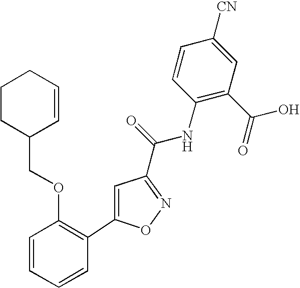

Prepared according to General method B: 5-[2-(cyclohex-2-en-1-ylmethoxy)phenyl]isoxazole-3-carboxylic acid (370 mg, 1.2 immol) and tert-butyl-2amino-5-cyano benzoate (299 mg, 1.4 mmol) afforded 200 mg (32%) of t-butyl ester as a white solid, 190 mg of which was hydrolyzed by TFA (2 ml) in DCM (10 ml) to afford 158 mg (94%) of a white solid.

Analytical data

¹H NMR (400 MHz, DMSO-d₆) δ 12.76 (s, 1H), 8.86 (d, J=8.9 Hz, 1H), 8.43 (d, J=2.1 Hz, 1H), 8.14 (dd, J=2.3, 8.9 Hz, 1H), 7.76 (dd, J=1.7, 7.9 Hz, 1H), 7.55 (m, 1H), 7.30 (d,J=8.3 Hz, 1H), 7.22 (s, 1H), 7.16 (rm, 1H), 5.72 (s, 2 H), (d, J=6.2 Hz, 2 H), 2.21 (m, 2 H), 2.10 (m, 2 H), 1.92 (m, 2 H), 1.43 (m, 1H).

Preparation of 5- [2-(Carboxymethoxy)phenyl]isoxazole-3-carboxylic acid

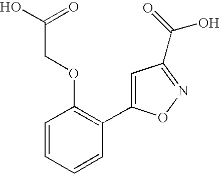

Prepared according to General method C: ethyl-5-(2-hydroxyphenyl)isoxazole-3-carboxylate (500 mg, 2.15 mmol) and 4-(chloroacetyl)morpholine (529 mg, 3.2 mmol) afforded 450 mg (56%) of ethyl 5-[2-(2-morpholin-4-yl-2oxoethoxy)phenyl]isoxazole-3-carboxylate as a white solid, which was hydrolyzed in MeOH (10 ml) and water (5 ml) by LiOH.H₂O (300 mg, 7.1 mmol) to afford 380 mg (100%) of 5-[2-(carboxymethoxy)phenyl]isoxazole-3-carboxylic acid as a white solid.

Analytical data

¹H NMR (400 MHz, DMSO-d₆) δ 7.95 (dd, J=1.7, 7.9 Hz, 1H), 7.62 (m, 1H), 7.61 (s, 1H), 7.17 (m, 2 H), 4.92 (s, 2 H).

### Example 7.65: 2-({[2-(3-{[(2-Carboxy-4-cyanophenyl)aminolcarbonyl}isoxazol-5-yl)phenoxyl acetyl}amino)-5-cyanobenzoic acid

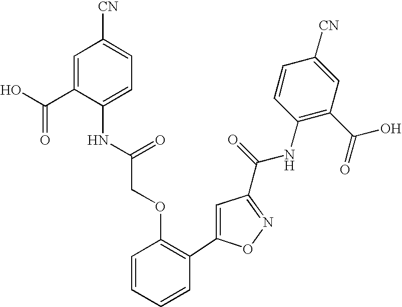

Prepared according to General method B: 5-[2-(carboxymethoxy)phenyl]isoxazole-3-carboxylic acid (380 mg, 1.4 mmol) and tert-butyl-2-amino-5-cyano benzoate (693 mg, 3.2 rumol) afforded 780 mg (89%) of t-butyl ester as a light yellow solid, 290 mg of which was hydrolyzed by TFA (2 ml) in DCM (10 ml) to afford 260 mg (99%) of a white solid.

Analytical data

¹H NMR (300 MHz, DMSO-d₆) δ 12.72 (s, 1), 12.05 (s, 1H), 8.84 (dd, J=3.0, 8.9 Hz, 2 H), 8.43 (d, J=2.1 Hz, 1H), 8.30 (2.1, J=1Hz, H), 8.15 (dd, J=1.9, 8.9 Hz, 1H), 8.09 (dd, J=2.1, 8.9 Hz, 1H), 8.01 (dd, J=1.3, 7.7 Hz, 1H), 7.56 (t, J=8.3 Hz, 1H), 7.53 (s, 1H), 7.27 (d, J=8.3 Hz, 1), 7.23 (t, J=7.7 Hz, 1H), 5.19 (s, 2 H).

Preparation of 5-12-(1-Ethylpropoxy)phenyl]isoxazole-3-carboxylic acid

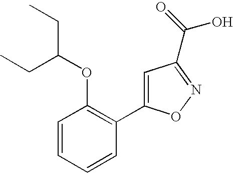

Prepared according to General method D: ethyl-5-(2-hydroxyphenyl)isoxazolecarboxylate (500 mg, 2.15 mmol) and pentan-3-ol (189 mg, 2.15 mmol) afforded 330 mg (51%) of ethyl 5-[2-(1-ethylpropoxy)phenyl]isoxazole-3-carboxylate as a white soild, which was hydrolyzed in MeOH (10 ml) and water (5 ml) by LiOH.H₂O (300 mg, 7.1 mmol) to afford 240 mg (80%) of 5-[2-(1-ethylpropoxy)phenyl]isoxazole-3-carboxylic acid as a yellow oil.

Analytical data

¹H NMR (300 MHz, DMSO-d₆) δ 7.92 (dd, J=1.7, 7.7 Hz, 1H), 7.50 (m, 1H), 7.26 (d, J=8.3 Hz, 1), 7.10 (t, J=7.2 Hz, 1H), 7.05 (s, 1H), 4.55 (pent, J=5.7 Hz, 1 H), 1.72 (m, 4 H), 0.91 (t, J=7.4 Hz, 6 H).

### Example 7.66: 5-Cyano-2-[({5-12-(1-ethylpropoxy)phenyl]isoxazol-3-yl}carbonyl)amino]benzoic acid

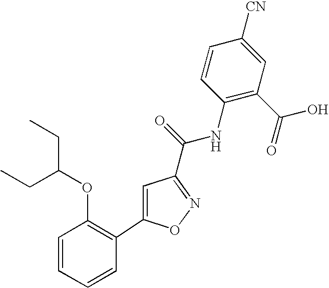

Prepared according to General method B: 5-[2-(1-ethylpropoxy)phenyl]isoxazole-3-carboxylic acid (240 mg, 0.9 mmol) and tert-butyl-2-amino-5-cyano benzoate (299 mg, 1.0 mmol) afforded 150 mg (36%) of t-butyl ester as a yellow solid, which was hydrolyzed by TFA (2 ml) in DCM (10 ml) to afford 120 mg (91%) of a white solid.

Analytical data

¹H NMR (400 MHz, DMSO-d₆) δ 12.78 (s, 1H), 8.85 (d, J=8.9 Hz, 1H), 8.43 (d, J=2.1 Hz, 1H), 8.14 (dd, J=2.1, 8.7 Hz, 1H), 7.96 (dd, J=1.7, 7.9 Hz, 1H), 7.53 (m, 1H), 7.30 (d, J=8.5 Hz, 1H), 7.20 (s, 1H), 7.12 (m1, 1 H), 4.58 (pent, J=Hz, 1H), 1.74 (m, 4 H), 0.93 (t, J=7.3 Hz, 6 H).

Preparation of 5-{2-12-(4-Methyl-1,3-thiazol-5-yl)ethoxylphenyl}isoxazole-3-carboxylic acid

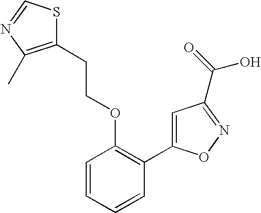

Prepared according to General method D: ethyl-5-(2-hydroxyphenyl)isoxazole-3-carboxylate (500 mg, 2.15 nmol) and 2-(4-methyl-1,3-thiazol-5-yl)ethanol (307 mg, 2.15 mmol) afforded 470 mg (62%) of ethyl 5-{2-[2-(4-methyl-1,3-thiazol-5-yl)ethoxy]phenyl}isoxazole-3-carboxylate as a white solid, which was hydrolyzed in MeOH (100 ml) and water (5 ml) by LiOH.H₂O (300 mg, 7.1 mmol) to afford 90 mg (21%) of 5-{2-[2-(4-methyl-1,3-thiazol-5-yl)ethoxy]phenyl}isoxazole-3-carboxylic acid as a white solid.

Analytical data

¹H NMR (300 MHz, DMSO-d₆) δ 8.82 (s, 1H), 7.90 (dd, J=1.5, 7.7 Hz, 1H), 7.53 (rm, 1H), 7.28 (d, J=8.3 Hz, 1H), 7.14 (t, J=7.4 Hz, 1H), 6.96 (s, 1H), 4.39 (t, J=6.0 Hz, 2 H), 3.35 (t, J=6.0 Hz, 2 H), 2.36 (s, 3 H).

### Example 7.67: 5-Cyano-2-{[(5-{2-12-(4-methyl-1,3-thiazol-5-yl)ethoxylphenyl}isoxazol amino}benzoic acid

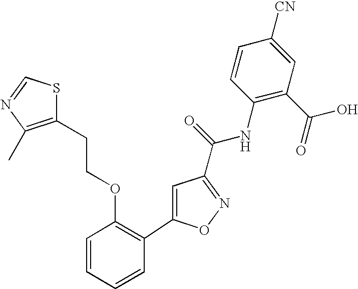

Prepared according to General method B: 5-{2-[2-(4-methyl-1,3-thiazol-5-yl)ethoxy]phenyl}isoxazole-3-carboxylic acid (90 mg, 0.27 mmol) and tert-butyl-2-amino-5-cyano benzoate (66 mg, 0.3 mmol) afforded 60 mg (42%) of t-butyl ester as a yellow solid, which was hydrolyzed by TFA (2 ml) in DCM (10 ml) to afford 48 mg (89%) of a white solid.

Analytical data

¹H NMR (400 MHz, DMSO-d₆) δ 12.78 (s, 1H), 8.86 (d, J=8.7 Hz, 1H), 8.82 (s, 1 H), 8.44 (d, J=2.1 Hz, 1H), 8.15 (dd, J=2.1, 8.7 Hz, 1H), 7.94 (dd, J=1.7, 7.9 Hz, 1H), 7.56 (m, 1H), 7.31 (d, J=8.3 Hz, 1H), 7.17 (t, J=7.1 Hz, 1H), 7.12 (s, 1 H), 4.42 (t, J=6.2 Hz, 2 H), 1.38 (t, J=6.0 Hz, 2 H), 2.38 (s, 3 H).

Preparation of 5-12-(1-Cyclohexylethoxy)phenyl]isoxazole-3-carboxylic acid

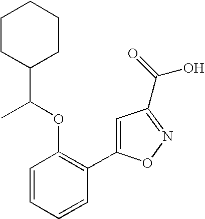

Prepared according to General method D: ethyl-5-(2-hydroxyphenyl)isoxazole-3-carboxylate (500 mg, 2.15 mmol) and 1-cyclohexylethanol (275 mg, 2.15 mmol) afforded 340 mg (47%) of ethyl 5-[2-(1-cyclohexylethoxy)phenyl]isoxazole-3-carboxylate as a yellow oil, which was hydrolyzed in MeOH (10 ml) and water (5 ml) by LiOH.H₂O (300 rng, 7.1 mmol) to afford 280 mg (89%) of 5-[2-(1cyclohexylethoxy)phen acid as a yellow oil.

Analytical data

¹H NMR (400 MHz, DMSO-d₆) δ 7.90 (dd, J=1.7, 7.7 Hz, 1H), 7.50 (m, 1H), 7.27 (d, J=8.3 Hz, 1H), 7.09 (m, 1H), 7.08 (s, 1H), 4.55 (m, 1H), 1.88 (d, J=11.8 Hz, 1H), 1.69(m,4H), 1.26(d,J=6.2 Hz,3H), 1.16(m,6H).

### Example 7.68: 5-Cyano-2-[({5-[2-(1-cyclohexylethoxy)phenyl]isoxazol-3-yl}carbonyl)amino]benzoic acid

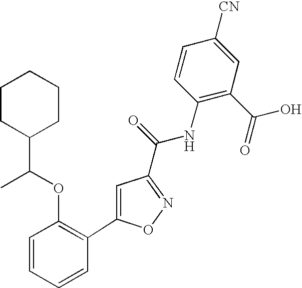

Prepared according to General method B: 5-[2-(1-cyclohexylethoxy)phenyl]isoxazole-3-carboxylic acid (280 mg, 0.9 mmol) and tert-butyl-2-amino-5-cyano benzoate (215 mg, 1.0 mmol) afforded 190 mg (42%) of t-butyl ester as a yellow solid, 80 mg of which was hydrolyzed by TFA (2 ml) in DCM (10 ml) to afford 61 mg (86%) of a white solid.

Analytical data

1H NMR (400 MHz, DMSO-d₆) δ 12.75 (s, 1H), 8.86 (d, J=8.9 Hz, 1), 8.43 (d, J=2.1 Hz, 1H), 8.14 (dd, J=2.1, 8.9 Hz, 1H), 7.94 (dd, J=1.7, 7.9 Hz, 1H), 7.53 (m, 1H), 7.30 (d, J=8.5 Hz, 1H), 7.23 (s, 1H), 7.11 (m, 1H), 4.59 (pent, J=5.8 Hz, 1H), 1.91 (d, J=12.0 Hz, 1H), 1.73 (m, 4 H), 1.28 (d, J=6.2 Hz, 3 H), 1.16 (m, 6H).

Preparation of 5-[2-(2-Oxobutoxy)phenyl]isoxazole-3-carboxylic acid

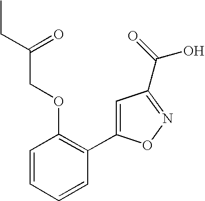

Prepared according to General method C: ethyl-5-(2-hydroxyphenyl)isoxazole-3-carboxylate (500 mg, 2.15 mmol) and 1-bromobutan-2-one (649 mg, 4.30 mmol) afforded 650 mg (100%) of ethyl 5-[2-(2-oxobutoxy)phenyl]isoxazole-3-carboxylate as a yellow oil, 580 mg of which was hydrolyzed in MeOH (10 ml) and water (5 ml) by LiOH.H₂O (300 mg, 7.1 mmol) to afford 520 mg (99%) of 5-[2-(2oxobutoxy)phenyl]isoxazole-3-carboxylic acid as a yellow oil.

Analytical data

¹H NMR (300 MHz, DMSO-d₆) δ 7.95 (dd, J=1.7, 8.1 Hz, 1H), 7.55 (s, 1H), 7.61 (m, 1H), 7.15 (m, 2 H), 5.11 (s, 2 H), 2.55 (tetra, J=7.4 Hz, 2 H), 1.02 (t, J=7.4 Hz, 3 H).

### Example 7.69: 5-Cyano-2-[({5-[2-(2-oxobutoxy)phenyl]isoxazol-3-yl}carbonyl)amino]benzoic acid

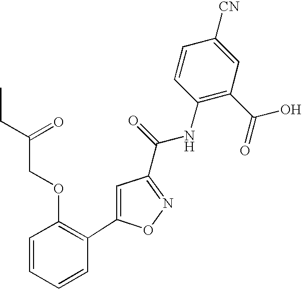

Prepared according to General method B: 5-[2-(2-oxobutoxy)phenyl]isoxazole-3-carboxylic acid (520 mg, 1.9 mmol) and tert-butyl-2-amino-5-cyano benzoate (453 mg, 2.1 imtol) afforded 470 mg (52%) of t-butyl ester as a yellow solid, 170 mg of which was hydrolyzed by TFA (2 ml) in DCM (10 ml) to afford 140 mg (93%) of a yellow solid.

Analytical data

¹H NMR (400 MHz, DMSO-d₆) δ 12.75 (s, 1H), 8.87 (d, J=8.9 Hz, 1H), 8.43 (d, J=2.1 Hz, 1H), 8.15 (dd, J=2.1, 8.7 Hz, 1H), 7.98 (dd, J=1.7, 8.1 Hz, 1H), 7.68 (s, 1H), 7.63 (m, 1H), 7.18 (m, 2 H), 5.14 (s, 2 H), 2.56 (tetra, J=7.5 Hz, 2 H), 1.03 (t, J=7.3 Hz, 3 H).

Preparation of 5-[2-(2-Oxo-2-phenylethoxy)phenyl]isoxazole-3-carboxylic acid

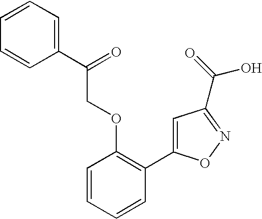

Prepared according to General method C: ethyl-5-(2-hydroxyphenyl)isoxazole-3-carboxylate (500 mg, 2.15 mmol) and 2-bromo-1-phenylethanone (513 mg, 2.58 mmol) afforded 650 mg (86%) of ethyl 5-[2-(2-oxo-2-phenylethoxy)phenyl]isoxazole-3-carboxylate as a yellow oil, which was hydrolyzed in MeOH (10 ml) and water (5 ml) by LiOH.H₂O (300 mg, 7.1 mmol) to afford 610 mg (100%) of 5-[2-(2-oxo-2-phenylethoxy)phenyl]isoxazole-3-carboxylic acid as a yellow solid.

Analytical data

¹H NMR (300 MHz, DMSO-d₆) δ 8.11 (d, J=7.2 Hz, 2 H), 7.99 (dd, J=1.5, 7.7 Hz, 1H), 7.75 (s, 1H), 7.71 (d, J=7.4 Hz, 1H), 7.6 (m, 2 H), 7.53 (m, 1H), 7.37 (d, J=8.1 Hz, 1H), 7.18 (t, J=7.7 Hz, 1H), 5.84 (s, 2 H).

### Example 7.70: 5-Cyano-2-[({5-[2-(2-oxo-2-phenylethoxy)phenyl]isoxazol-3-yl}carbonyl)amino]benzoic acid

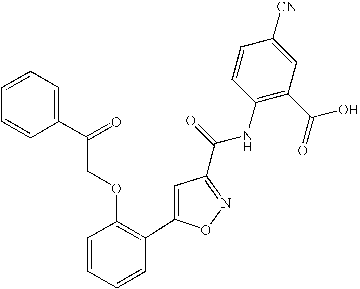

Prepared according to General method B: 5-[2-(2-oxo-2phenylethoxy)phenyl]isoxazole-3-carboxylic acid (610 mg, 1.9 mmol) and tert-butyl-2amimo-5-cyano benzoate (453 mg, 2.1 mmol) afforded 600 mg (61%) of t-butyl ester as a yellow solid, 300 mg of which was hydrolyzed by TFA (2 ml) in DCM (10 ml) to afford 190 mg (100%) of a white solid.

Analytical data

¹H NMR (300 MHz, DMSO-d₆) δ 12.78 (s, 1H), 8.89 (d, J=8.7 Hz, 1H), 8.44 (d, J=1.9 Hz, 1H), 8.16 (dd, J=2.1, 8.7 Hz, 1H), 8.12 (tetra, J=7.2 Hz, 2 H), 8.03 (dd, J=1.7, 7.9 Hz, 1H), 7.88 (s, 1H), 7.73 (m, 1H), 7.61 (t, J=7.7 Hz, 2 H), 7.56 (m, 1H), 7.39 (d, J=8.3 Hz, 1H), 7.20 (t, J=7.7 Hz, 1H), 5.88 (s, 2 H).

Preparation of 5-{2-[(5-Hydroxypentyl)oxylphenyl}isoxazole-3-carboxylic acid

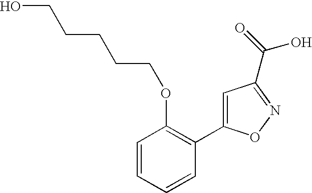

Prepared according to General method D: ethyl-5-(2-hydroxyphenyl)isoxazole-3-carboxylate (500 mg, 2.15 mmol) and 5-{[tert-butyl(dimethyl)silyl]oxy}pentan-1-ol (469 mg, 2.15 mmol) afforded 640 mg (69%) of ethyl 5-{2-[(5-{[tertbutyl(dimethyl)silyl]oxy}pentyl)oxy]phenyl}isoxazole-3-carboxylate as a yellow oil, which was hydrolyzed in MeOH (10 ml) and water (5 ml) by LiOH.H₂O (500 mg, 11.9 mmol) to afford 210 mg (44%) of 5-{2-[(5-hydroxypentyl)oxy]phenyl}isoxazole-3-carboxy acid as a white solid.

Analytical data

¹H NMR (400 MHz, DMSO-d₆) δ 7.85 (dd, J=1.7, 7.7 Hz, 1H), 7.46 (m, 1H), 7.20 (d, J=8.1 Hz, 1), 7.09 (dt, J=0.8, 7.5 Hz, 1H), 6.91 (s, 1), 4.14 (t, J=6.3 Hz, 2 H), 3.41 (t, J=6.0 Hz, 2 H), 1.84 (pent, J=6.8 Hz, 2 H), 1.61 (pent, J=3.1 Hz, 4 H).

### Example 7.71: 2-{1(5-{2-[(5-Chloropentyl)oxylphenyl}isoxazol-3-yl)carbonyl]amino}-5-cyanobenzoic acid

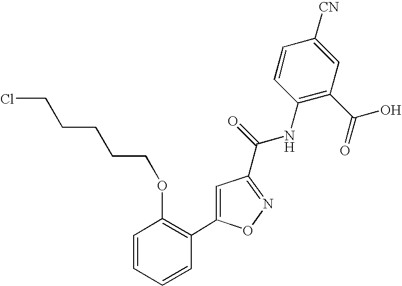

Prepared according to General method B: 5-{2-[(5 hydroxypentyl)oxy]phenyl}isoxazole-3-carboxylic acid (251 mg, 0.79 mmol) and tert butyl-2-amino-5-cyano benzoate (206 mg, 0.94 mmol) afforded 90 mg (21%) of t-butyl ester as a white solid, which was hydrolyzed by TFA (2 ml) in DCM (10 ml) to afford 62 mg (79%) of a white solid.

Analytical data

¹H NMR (300 MHz, DMSO-d₆) δ 12.76 (s, 1H), 8.85 (d, J=8.9 Hz, 1H), 8.44 (d, J=2.1 Hz, 1H), 8.15 (dd, J=2.1, 8.9 Hz, 1H), 7.95 (dd, J=1.5, 7.7 Hz, 1H), 7.55 (m, 1H), 7.29 (d, J=8.5 Hz, 1H), 7.22 (s, 1), 7.15 (t, J=7.4 Hz, 1H), 4.21 (t, J=6.2 Hz, 2H), 3.68 (t,J=6.6 Hz, 2 H), 1.83 (m, 4 H), 1.61 (m, 2 H).

### Example 8: R₄ as Benzoxazole and derivatives thereof

Preparation of 2-Amino-5-cyanobenzoic acid

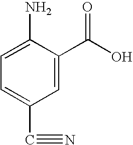

To a slurry of methyl 2-amino-5-cyanobenzoate (9.55 g, 54.2 mmol) in methanol (200 mL) was added aqueous sodium hydroxide (30 mL, 6.0 molar, 180 mmol). This total of 27.5 hours, the solvent was evaporated, and the residue was dissolved in water (300 mL). This solution was washed with methylene chloride (2×200 mL) and then acidified with concentrated HCl. The product was extracted into ethyl acetate (500 mL). The organics were dried over MgSO₄ and evaporated leaving the product as 8.57 g (97.5%) of yellow solid that was used without further purification.

Preparation of Benzyl 2-amino-5-cyanobenzoate

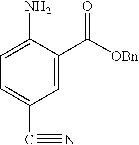

To a flask containing 2-amino-5-cyanobenzoic acid (8.57 g, 52.9 mmol) and cesium carbonate (17.2 g, 52.8 mmol) was added DMF (150 mL). Benzyl bromide (6.00 mL, 50.4 mmol) was added, resulting in the rapid formation of a precipitate. The mixture was stirred at room temperature for 6 hours and then added to a separatory funnel with methyl tert-butyl ether (500 mL). This mixture was washed with water (250 mL), saturated aqueous sodium bicarbonate (2×250 mL), and brine (250 mL). The organics were then filtered through a 1″ plug of silica gel. The plug was rinsed with methylene chloride, and the combined organics were evaporated leaving the product as 12.5 g of yellow solid (98.0% from benzyl bromide, 93.6% from acid). ¹H NMR (400 MHz, DMSO-d₆) δ 8.09 (d, J=2.1 Hz, 1H), 7.59 (dd, J=8.7, 2.1 Hz, 1), 7.45-7.53 (m, 4 H), 7.41 (t, J=7.2 Hz, 2 H), 7.36 (t, J=7.1 Hz, 1H), 6.88 (d, J=8.9 Hz, 1H), 5.32 (s, 2 H).

Preparation of Benzyl 1,3-benzoxazole-2-carboxylate

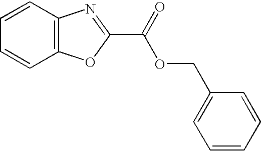

To a stirred solution of benzoxazole (1.32 g, 11.1 mmol, Aldrich) in THF (20 μL) at 40° C. was added n-BuLi (6.5 mL of 1.6 M in hexanes, 10.4 mmol, Aldrich). The solution was stirred for 40 minutes at −40° C. and then added by cannula to a solution of benzyl cyanoformate (1.67 g, 10.4 mmol, Aldrich) in THF (20 mL) at −78° C. The resulting mixture was stirred at -78 uC for 10 minutes, at 40° C. for 10 minutes, and then at room temperature for 20 minutes. It was then added to a separatory funnel with 200 mL of saturated aqueous NaHCO₃. Product was extracted into 200 μL of EtOAc. The EtOAc was washed with another 200 mL of saturated NaHCO₃ and 200 mL of brine. The EtOAc was dried over Na₂SO₄ and evaporated leaving an orange oil. Product was purified by two successive chromatography runs using a Biotage Flash 40 M silica cartridge with 60% CH₂Cl₂ in heptane as eluent. Yield was 473 mg of yellow solid.

### Example 8.1: 2-[(1,3-Benzoxazol-2-ylcarbonyl)amino]-5-cyanobenzoic acid

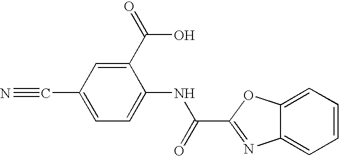

To a solution of benzyl 1,3-benzoxazole-2-carboxylate (233 mg, 0.920 mmol) in 1:1 ethanol/THF (20 mL) was added palladium on carbon (56 mg of 5%, Aldrich) and triethylamine (180 μL, 1.29 mmol, Aldrich). The mixture was stirred under 1 ATM of hydrogen for 2 hours and then filtered through a plug of celite. Removal of the solvent left the triethylamine salt as an orange oil (the protonated form of the acid rapidly decarboxylates and should be avoided). This oil was dissolved in CH₂Cl₂ (20 mL) and treated with DMF (20 μL) followed by oxalyl chloride (220 [L, 2.52 mmol, Aldrich). Solvent and excess oxalyl chloride were removed by rotary evaporation after 76 hours. The residue was dissolved in CH₂Cl₂ (20 mL), and benzyl 2-amino-5-cyanobenzoate (250 mg, 0.991 mmol) in pyridine (8 mL) was added. The mixture was stirred overmght and then added to a separatory funnel with 100 mL of CH₂Cl₂. This solution was washed with 2×100 of 1.0 M HCl and 100 mL of brine. Product was adsorbed onto silica gel and purified on a Biotage Flash 40 M siliga gel cartridge using CH₂Cl₂ as eluent. Product was collected as 218 mg of white solid as the benzyl ester. A mixture of benzyl 2-[(1,3-benzoxazol-2-ylcarbonyl)amino]-5-cyanobenzoate (168 mg, 0.423 mmol) and palladium on carbon (33 mg of 5%, Aldrich) in 2:1 THF/ethanol (30 mL) was stirred under 1 ATM of hydrogen for 25 minutes. The mixture was filtered through a plug of celite and then evaporated. The residue was dried at 100° C. under vacuum yielding 116 mg of white solid. ¹H NMR (400 MHz, DMSO-D6) δ ppm 7.56 (t, J=7.67 Hz, 1H) 7.63 (t, J=7.88 Hz, 1H) 7.94 (d, J=8.29 Hz, 1H) 8.00 (d, J=7.67 Hz, 1H) 8.16 (dd, J=8.81, 1.97 Hz, 1H) 8.45 (d, J=2.07 Hz, 1H) 8.87 (d, J=8.71 Hz, 1H) 13.16 (s, 1H).

### Example 9: R₄ as Pyrazine and Derivatives Thereof

### Example 9.1: 5-Cyano-2-({[5-(2-phenylethyl)pyrazin-2-yl]carbonyl}amino)benzoic acid

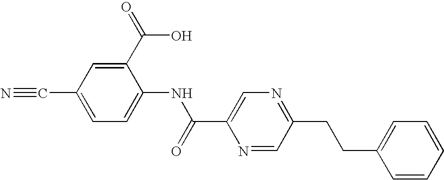

To a slurry of 5-[(E)-2-phenylethenyl]pyrazine-2-carboxylic acid (146 mg, 0.645 mmol, from RCC) in CH₂Cl₂ (15 mL) was added DMF (10 μL) followed by oxalyl chloride (100 μL, 1.15 mmol, Aldrich). An orange solution quickly resulted. Solvent and excess oxalyl chloride were removed by rotary evaporation after stirring overmght. The residue was dissolved in CH₂Cl₂ (15 mL), and benzyl 2-amino-5-cyanobenzoate (155 mg, 0.614 mmol) in pyridine (8 mL) was added. The mixture was stirred overnight and then added to a separatory finmel with 100 mL of CH₂Cl₂. This solution was washed with 2×100 of 1.0 M HCl and 100 mL of brine. Product was adsorbed onto silica gel and purified on a Biotage Flash 40 M silica gel cartridge using a gradient from CH₂Cl₂ to 2% EtOAc in CH₂Cl₂ as eluent. Product was collected as 138 mg of yellow solid as the benzyl ester. A mixture of benzyl

5-cyano-2-[({5-[(E)-2phenylethenyl]pyrazin-2-yl}carbonyl)amino]benzoate (118 mg, 0.256 mmol) and palladium on carbon (46 mg of 5%, Aldrich) in 1:1 THF/ethanol (40 mL) was stirred under 1 ATM of hydrogen for 6.5 hours. The mixture was filtered through a plug of celite and then evaporated. The residue was recrystallized from ethanol/THF. The solids were washed with ethanol followed by heptane and then dried at 100° C. under vacuum yielding 37 mg of tan solid. ¹H NMR (400 MHz, DMSO-D6) δ ppm 3.08 (t, J=7.88 Hz, 2 H) 3.25 (t, J=7.77 Hz, 2 H) 7.24 (m, 5 H) 8.12 (dd, J=8.81, 1.97 Hz, 1 H) 8.42 (d, J=1.87 Hz, 1H) 8.70 (d, J=1.04 Hz, 1H) 8.98 (d, J=8.71 Hz, 1H) 9.28 (d, J=1.24 Hz, 1H) 13.28 (s, 1H).

Preparation of 5-(Hexyloxy)pyrazine-2-carboxylic acid

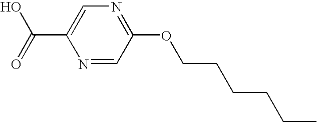

Hexyl alcohol (30 mL, Aldrich) was added to a flask containing sodium (423 mg, 18.4 mmol), and the mixture was heated in a 60° C. oil bath for 45 minutes to dissolve the sodium. Methyl 5-chloropyrazine-2-carboxylate (1.62 g, 9.39 mmol, Lonza) was added, and the mixture was stirred in a 50° C. oil bath for 35 minutes. The mixture was added to a separatory funnel with 100 mL1 of CH₂Cl₂. This was washed with 100 mL of water, but solid material was present in the CH₂Cl₂ that would not dissolve. The CH₂Cl₂ was then washed with 100 mL of 1.0 M aqueous HCl, and everything dissolved. The CH₂Cl₂ was washed with another 100 mL of water, dried over MgSO₄, and evaporated. The residual hexanol was distil]ed away, and the residue was purified on a Biotage Flash 40 M silica cartridge. Hexyl 5-hexyloxypyrazine-2-carboxylate eluted with CH₂Cl₂ followed by methyl 5-hexyloxypyrazine-2-caboxylate. Addition of methanol to the eluent resulted in the elution of 5-(hexyloxy)pyrazine-2-carboxylic acid followed by 5-chloropyrazine-2-carboxylic acid. The hexyloxy acid was isolated as 137 mg of white solid. Hexyl 5-hexyloxypyrazine-2-carboxylate was isolated as 1.64 g of colorless oil.

### Example 9.2: 5-Cyano-2-({[5-(hexyloxy)pyrazin-2-yl]carbonyl}amino)benzoic acid

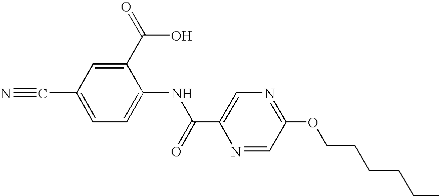

To a mixture of 5-(hexyloxy)pyrazine-2-carboxylic acid (271 mg, 1.21 mmol) in CH₂Cl₂ (15 mL) was added DMF (10 μL) followed by oxalyl chloride (160 μL, 1. 83 mmol, Aldrich). Solvent and excess oxalyl chloride were removed by rotary evaporation after 2 hours. The residue was dissolved in CH₂Cl₂ (15 mL), and benzyl 2-amino-5-cyanobenzoate (288 mg, 1.14 mmol) in pyridine (6 mL) was added. The mixture was stirred overnight and then added to a separatory fumnel with 100 mL of CH₂Cl₂. This solution was washed with 2×100 of 1.0 M HCl and 100 μL of brine. cartridge using CH₂Cl₂ as eluent. Product was collected as 205 mg of white solid as the benzyl ester. A mixture of benzyl

5-cyano-2-({[5-(hexyloxy)pyrazin-2-yl]carbonyl}amino)benzoate (169 mg, 0.369 mmol) and palladium on carbon (25 mg of 5%, Aldrich) in 6:1 THF/ethanol (35 mL) was stirred under 1 ATM of hydrogen for 1 hour. The mixture was filtered through a plug of celite and then evaporated. The residue was twice suspended in methanol and evaporated. It was then suspended in CH₂Cl₂ and evaporated. It was then dried at 100° C. under vacuum yielding 122 mg of tan solid. ¹H NMR (400 MHz, DMSO-D6) δ ppm 0.88 (t, J=7.05 Hz, 3 H) 1.32 (m, 4 H) 1.42(m,2H) 1.77(m,2H)4.40(t,J=6.63 Hz,2H)8.11 (dd,J=8.81, 2.18 Hz, 1 H) 8.40 (d, J=2.07 Hz, 1H) 8.42 (d, J=1.24 Hz, 1H) 8.93 (d, J=1.24 Hz, 1H) 8.98 (d, J=8.91 Hz, 1H) 13.07 (s, 1H).

### Example 10: R₄ as Oxazoles and Derivatives Thereof

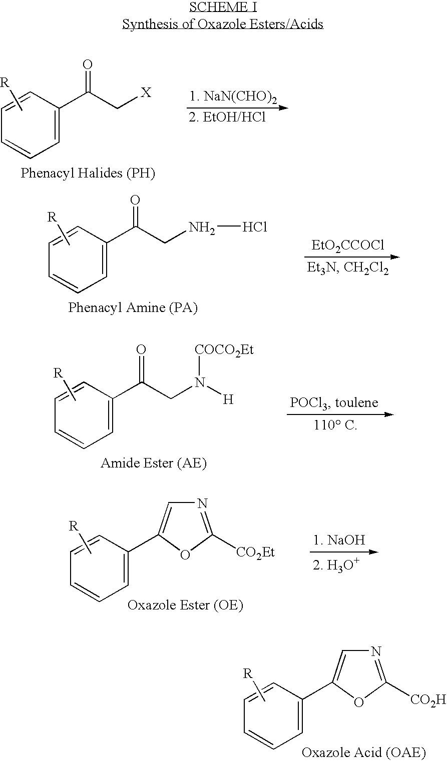

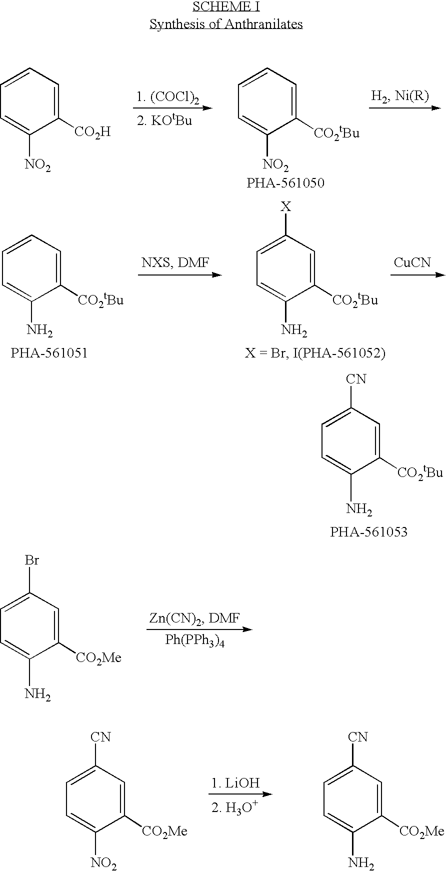

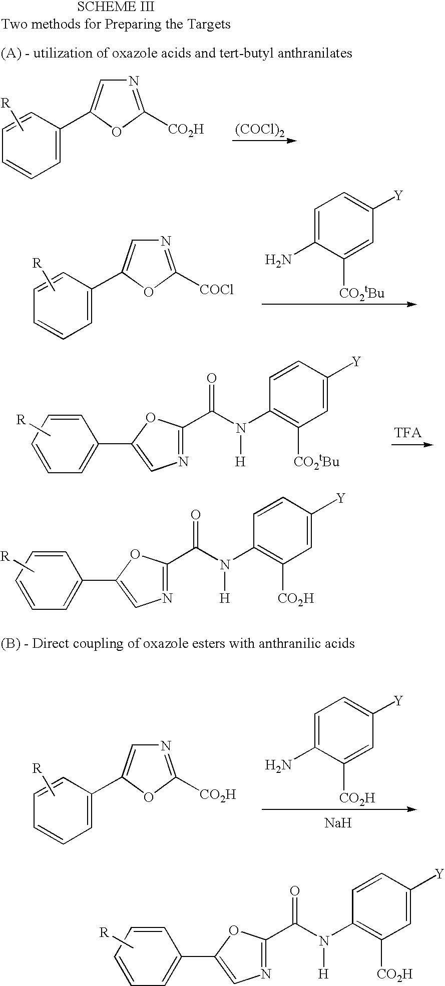

### PREPARATION OF PHENACYL HALIDES

Method A—bromination of methyl ketones

2-Bromo-3′, 4′-methylenedioxvacetophenone (PH-k):

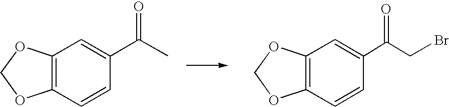

3,4-Methylenedioxyacetophenone (20.0 g, 121.2 mmol) was dissolved in CHCl₃/EtOAc (700 mUL700 mL) followed by the addition of CuBr₂ (40.6 g, 181.8 mmol, 1.5 eq). The reaction mixture was heated at reflux for 3 days. TLC (1/1 Hept/EtOAc) showed about 80% cormpletion (product and S.M have close Rf). Solid was removed by filtration, washed with CHCl₃. The filtrate was concentrated on a rotavap to give an orange solid (27.77 g, 94% yield, containing 21% unreacted starting material). The material iwas carried to the next step without flirther purification. ¹H NMR (300 MHz, CDCl₃) 6 7.60 (d, 1H), 7.40 (s, 1H), 6.85 (d, 1H), 6.20 (s, 2H), 4.40 (s, 2H).

Yield: 94%

2-Bromo-2′-(trifluoromethyl)acetophenone (PH-g:

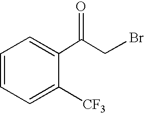

¹HNMR (300 MHz, DMSO-d₆) δ 7.45-7.82 (m, 4H), 4.42 (s, 2 H). 92% yield (70% conversion)

4-(2-Bromo-acetyl)-benzonitrile (PH-l):

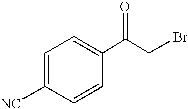

¹H NMR (300 MHz, CDCl₃) δ 8.19 (d, 1H), 7.80 (d, 1H), 4.45 (s, 2H).

Yield: 65%

3-(2-Bromo-acetyl)-benzonitrile (PH-m):

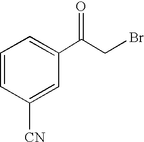

¹HNMR(300 MHz, DMSO-d₆) δ 8.53 (s, 1H), 8.29 (d, 1H),8.14 (d, 1H), 7.74 (t, 1 H), 5.03 (s, 2 H). 92% yield (75% conversion).

Method B—acylation of a aromatic heterocylcle with bromoacetyl bromide

2-Bromo-1-(1-methyl-1H-pyrrol-2-yl)-ethanone (PH-s): CH₃ CH₃

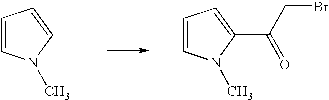

N-methylpyrrole (log, 123 mmol) was dissolved in 400 mL of dichloromethane. Bromoacetylbromide (1 eq) was added, and the solution stirred at 0° C. for 4.5 h. The reaction mixture was washed with 10% NaHCO₃ (2−1 L) and H₂O (2×100 mL). The organic layer was dried over Na₂SO₄ and concentrated under vacuum. Chromatography gave 4.9 g. ¹H NMR (300 MHz, CDCl₃) 6 7.15 (d, 1H), 6.90 (d, 1H), 6.18 (d, 1H), 4.30 (s, 2H), 3.98 (s, 3H).

Yield: 20%

### PREPARATION OFPHENACYL AMINES

Method A—using diformylimide sodium salt

2-Amino-1-(4-methoxy-phenyl)-ethanone hydrochioride(PA-b):

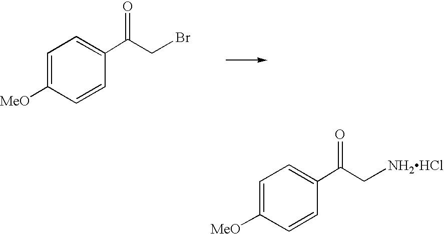

To a stirred solution of 2-Bromo-4′-methoxyacetophenone (20.0 g, 87.3 mmol) in CH₃CN (90 mL), was added sodium diformylamide (9.95 g, 104.76 mmol, 1.2 eq). The resulting mixture was stirred for 2 h at RT, and heated for 2 h at 70° C. (monitored by TLC). Solvent was removed under reduced pressure. Then EtOH (250 mL) and Conc. HCl (40 mL) were added. The reaction mixture was refluxed for 1 h. Solvent was removed by rotavap. The crude product was suspended in iPr—OH (100 mL) and stirred at RT O/N. The off-white pure product was obtained by filtration (17.28 g, 98%). ¹H NMR (300 MHz, DMSO-d₆) δ 8.4 (br), 8.0 (d, 2H), 7.1 (d, 2H), 4.5 (s, 2H), 3.9 (s, 3H).

MS (APCI⁺) m/z 166 (M—HCl).

Yield: 98%

2-Amino-3′-methoxvacetonhenone hydrochloride (PA-a):

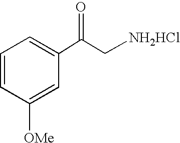

¹HNMR (300 MHz, DMSO-d₆) δ 8.50 (bs), 7.65 (d, 1H), 7.50 (m, 2 H), 7.32 (dd, 1 H), 4.59 (bs, 2H), 3.83 (s, 3 H). 86% yield

2-Amino-4′-fluoroacetoDhenone hydrochloride (PA-d):

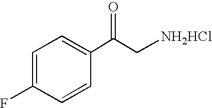

¹HNMR(300 Mz, DMSO-d₆) δ 8.50 (bs) (8.15 (m, 2 H), 7.44 (m, 2 H), 4.65 (m, 2 H). 76% yield

2-Amino-acetophenone hydroclioride (PA-f):

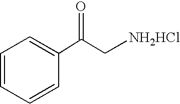

1HNMR (300 MHz, DMSO-d₆) δ 8.59 (bs), 8.06 (d, 2 H),7.67 (t, 1H), 7.55 (t, 2 H), 4.53 (s, 2 H). 72% yield.

2-Amino-2′-(trifluoromethyl)-acetoiphenone hydrochloride (PA-g):

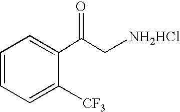

1HNMR (300 MHz, DMSO-d₆) δ 8.64 (bs),7.88-8.11 (m, 4 H), 4.35 (s, 2 H). 18% yield

2-Amino-1-furan-2-yl-ethanone hydrochloride (PA-h):

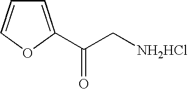

¹HNMR (300 WMz, DMSO-d₆) δ 8.35 (bs), 8.15 (d, 1H),7.67 (d, 1H), 6.82 (d, 1H), 4.32 (s, 2 H). 82% yield.

### -AMINO-2′-METHOXYACETOPHENONE HYDROCHLORIDE A-I):

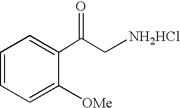

¹H NMR (300 MHz, DMSO-d₆) δ 8.31 (br), 7.85 (d, 1H), 7.70 (t, 1H), 7.27 (d, 1H), 7.12 (t, 1H), 4.35 (s, 2H), 3.95 (s, 3H).

Yield: 83%

2-Amino-3′, 4′-methylenedioxvacetouhenone hydrochloride (PA-k):

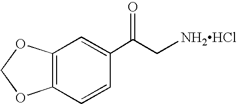

¹H NMR (300 MHz, DMSO-d₆) δ 8.39 (br), 7.65 (d, 1H), 7.55 (d, 1H), 7.13 (d, 1H), 6.20 (s, 2H), 4.5 (s, 2H).

Yield: 90%

2-Amino-4′-cyanoacetoihenone hydrochloride (PA-I):

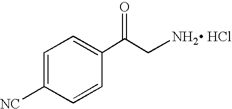

¹H NMR (300 MHz, DMSO-d₆) δ 8.60 (br), 8.17 (d, 2H), 8.10 (d, 2H), 4.70 (s, 2H). Yield: 59%

2-Amino-3′-cyanoacetoiphenone hydrochloride (PA-m):

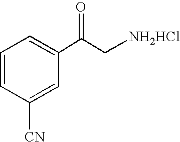

¹HNMR (300 MHz, DMSO-d₆) δ 8.71 (bs) 8.47 (s, 1H), 8.29 (d, 1H),8.20 (d, 1H), 7.82 (t, 1 F), 4.65 (d, 2 H). 77% yield.

2-Amino-1-(1-methyl-1H-nvrrol-2-yl)-ethanone hydrochloride (PA-s):

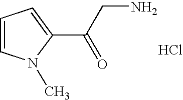

LC/MS: MS (APCI+) m/z 139(M-HCl-1).

Yield: 88%

### PREPARATION OF AMIDE ESTERS

Ethyl 12-(4-methoxyphenyl)-2-oxoethyl]amino-2-oxoethanoate (AE-b):

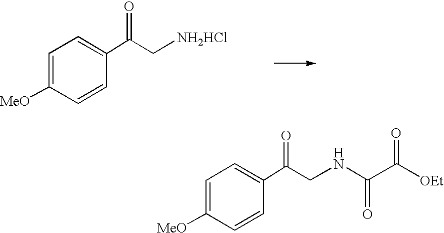

To a solution of 2-Amino-4′-methoxyacetophenone hydrochloride (17.28 g, 85.7 mmol) in DCM, was added triethylamine (25.1 mL, 179.97 mmol, 2.1 eq) followed by the addition of ethylchlorooxoacetate (9.7 mL, 86.6 mmol) slowly at 0° C. under N₂. The resulting mixture was stirred at 0° C. for another 2.5 h. The reaction was quenched with H₂O (200 mL). The organic phase was separated and the aqueous layer was extracted with DCM (2X50 mL). The combined organic layers were dried over MgSO₄. Solid was removed by filtration, and filtrate was concentrated to dryness under reduced pressure. The pure product was obtained by washing with Heptane/iPrOH (200 mL/20 mL) in the yield of 96%.

¹H NMR (300 MHz, CDCl₃) δ 8.1 (br), 8.0 (d, 2H), 7.0 (d, 2H), 4.8 (d, 2H), 4.4 (q, 2H), 3.9 (s, 3H), 1.4 (t, 3H).

MS (APCI⁺) m/z 266 (M+1).

Yield: 96%

Ethyl [2-(4-fluoronhenyl)-2-oxoethyl]amino-2-oxoethanoate (AE-d):

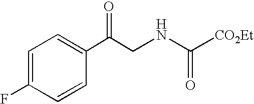

¹H NMR (300 MHz, CDCl₃) δ 9.18 (bs), 8.14 (t, 1H), 7.41 (mr, 2H), 4.71 (d, 2H), 4.35 (q, 2H), 1.32 (t, 3 H).

MS (APCI⁺) n/z 254(M +1). 80% yield

Ethyl [2-(2-trifluoromethylnhenyl)-2-oxoethyl]amino-2-oxoethanoate (AE-g):

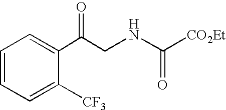

¹H NMR (300 MHz, CDCl₃) δ 9.38 (bs), 7.70-7.95 (m, 4H), 4.55 (d, 2H), 4.35 (q, 2H), 1.32 (t, 3 H). 90% yield.

N-(2-Furan-2-yl-2-oxo-ethyl)-oxalamic acid ethyl ester (AE-h):

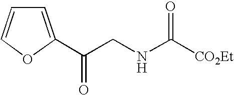

¹H NMR (300 MHz, CDCl₃) δ 9.15 (bs), 8.09 (d, 1H), 7.62 (d, 1H), 6.79 (d, 1H), 4.52 (d, 2H), 4.35 (q, 2H), 1.34 (t, 3 H). 85% yield.

Ethyl [2-(2-methoxyphenyl)-2-oxoethyl]amino-2-oxoethanoate (AE-i):

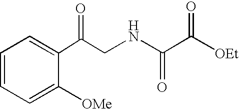

¹H NMR (300 MHz, CDCl₃) δ 8.15 (br), 7.98 (d, 1H), 7.60 (t, 1H), 7.30 (s, 1H), 7.05 (m, 1H), 4.80 (d, 2H), 4.45 (q, 2H), 4.00 (s, 3H), 1.45 (t, 3H).

Yield: 88%

Ethyl 12-(3,4-methylenedioxyphenvyl-2-oxoethyl]amino-2-oxoethanoate (AE-k):

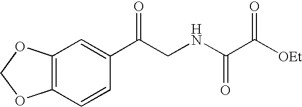

¹H NMR (300 MHz, CDCl₃) δ 8.10 (br), 7.60 (d, 1H), 7.45 (s, 1H), 6.90 (d, 1H), 6.10 (s, 2H), 4.75 (d, 2H), 4.40 (q, 2H), 1.40 (t, 3H). Yield: 96%

Ethyl 12-(4-cyanolhenyl)-2-oxoethyyl amino-2-oxoethanoate (AE-l):

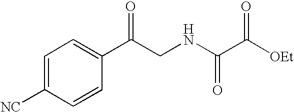

¹H NMR (300 MHz, CDCl₃) δ 8.10 (d, 2H), 7.85 (d, 2H), 4.85 (d, 2H), 4.40 (q, 2 H), 1.45 (t, 3H).

Yield: 66%

Ethyl 12-(3-cyanouhenyl)-2-oxoethyl]amino-2-oxoethanoate (AE-m):

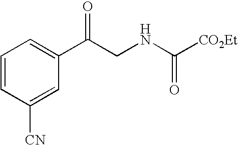

¹H NMR (300 MHz, CDCl₃) 8 9.24 (bs), 8.52 (s, 1H), 8.29 (d, 1H),8.14 (d, 11H), 7.74 (t, 1H), 4.78 (d, 2H), 4.35 (q, 2H), 1.32 (t, 2H). 81% yield.

N[-2-(1-Methyl-1H-nvrrol-2-yl)-2-oxo-ethyl]-oxalamic acid ethyl ester (AE-s):

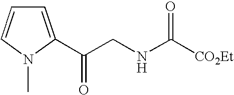

¹H NMR (300 MHz, DMSO-d₆) δ 9.01 (bs), 7.22 (m1, 2H), 6.18 (m1, 11H), 4.40 (d, 2H), 4.30 (q, 2H), 3.87 (s, 3H), 1.25 (t, 3H). 83% yield.

5-phenyl-oxazole-2-carboxylic acid ethyl ester (OE-f):

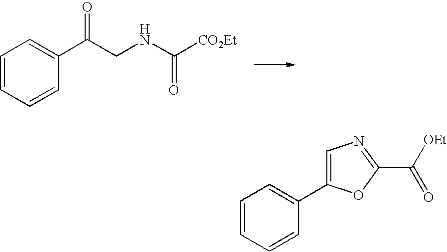

Ethyl [2-phenyl-2-oxoethyl]amino-2-oxoethanoate (16.5 g, 71 mmol) was dissolved in 200 mL of anhydrous toluene in a 1 L Parr bottle. To the solution was added 21 mL (225 mmol, 3.15 eq) of POC1₃. The Parr bottle was sealed, and the reaction mixture heated at an oil bath temperature of 110° C. for 48 h. The solvent and POC1₃ were removed under reduced pressure to give a brown oil. To the brown oil was added 300 mL H₂O. The aqueous solution was extracted twice with 150 mL of dichloromethane. The combined organics were dried over MgSO₄, filtered and the solvent removed in vacuo. The resulting brown solid was recrystallized from 50 mL of EtOH to give 12.5 g of an orange solid (80% yield).

¹H NMR (300 MHz, DMSO) 68.10 (s, 1H), 7.82 (d, 2 H), 7.55 (m, 3 H), 4.43 (q, 2H), 1.35 (t, 3H).

MS (APCI⁺) m/z 218(M +1)

5-(3-methoxyphenyl)-oxazole-2-carboxylic acid ethyl ester (OE-a):

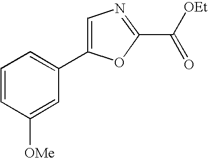

MS (APCI⁺) In/z 248(M+1).

Yield: 50%

5-(4-methoxylhenyl)-oxazole-2-carboxylic acid ethyl ester (OE-b):

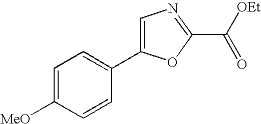

¹H NMR (300 MHz, CDCl₃) δ 7.7 (d, 2H), 7.4 (s, 1H), 7.0 (d, 2H), 4.5 (q, 2H), 3.8 (s, 3H), 1.5 (t, 3H).

MS (APCI⁺) m/z 248 (M+1).

Yield: 43%

5-(4-fluorophenyl)-oxazole-2-carboxylic acid ethyl ester (OE-d):

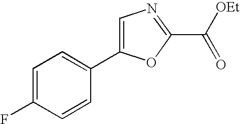

¹H NMR (300 MHz, DMSO) δ 8.05 (s, 1H), 7.85 (m, 2H), 7.41 (m, 2H), 4.42 (q, 2H), 1.35 (t, 3H). Yield: 59%

5-(2-trifluoromethylihenyl)-oxazole-2-carboxylic acid ethyl ester (OE-g):

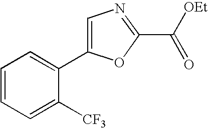

¹H NMR (300 MHz. DMSO) δ 7.67-8.05 (m, 5 H). 4.43 (q, 2H4). 1.35 (t, 3H)

MS (APCI⁺) m/z 286(M+1). 41% yield

5-furan-2-yl-oxazole-2-carboxylic acid ethyl ester (OE-h):

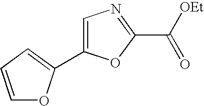

¹H NMR (300 MHz, DMSO) 6 7.95 (s, 1H),7.72 (d, 11H), 7.14(d, 1H), 6.79 (d, 11H), 4.37 (q, 2H), 1.38 (t, 3H).

MS (APCI⁺) mI/z 208(M+1). 35% yield

5-(2-Methoxyphenyl)-oxazole-2-carboxylic acid ethyl ester (OE-i):

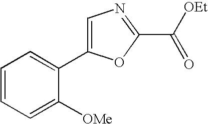

¹H NMR (300 MHz, CDCl₃) δ 7.90 (d, 1H), 7.70 (s, 1H), 7.38 (m, 1H), 7.10 (d, 1 ), 7.04 (t, 1H), 4.50 (q, 2H), 4.00 (s, 3H), 1.48 (t, 3H).

MS (APCI⁺) m/z 248(M+1).

Yield: 68%

5-(3,4-Methylenedioxyuhenyl)-oxazole-2-carboxylic acid ethyl ester (OE-k):

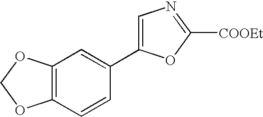

¹H NMR (300 MHz, CDCl₃) δ 7.42 (s, 1H), 7.27 (dd, 1H), 7.20 (d, 1H), 6.90 (d, 1H), 6.06 (s, 2H), 4.50 (q, 2H), 1.48 (t, 3H).

MS (APCI⁺) m/z 262(M+1).

Yield: 61%

5-(4-Cyanophenyl)-oxazole-2-carboxylic acid ethyl ester (OE-1):

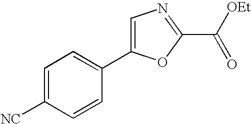

¹H NMR (300 MHz, CDCl₃) δ 7.90 (d, 2H), 7.78 (d, 2H), 7.68 (s, 1H), 4.55 (q, 2H), 1.50 (t, 3H).

MS (APCI⁺) m/z 243(M+1). Yield: 24%

5-(3-cyanonhenyl)-oxazole-2-carboxylic acid ethyl ester (OE-m):

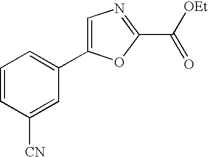

1H NMR (300 MHz, DMSO) δ 8.39 (s, 1H), 8.18 (m, 2 H), 7.92 (d, 1H), 7.74 (t, 1 H), 4.41 (q, 21H), 1.32 (t, 31H).

MS (APCI⁺) m/z 243(M+1). 35% yield

5-(1-methyl-1H-uvrrol-2-yl)-oxazole-2-carboxylic acid ethyl ester (OE-s):

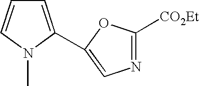

¹H NMR (300 MHz, DMSO) 6 7.58 (s, 1H), 7.05 (s, 1H), 6.58 (d, 1H), 6.15 (d, 1H), 4.41 (q, 2H), 3.83 (s, 3 H), 1.32 (t, 3 H). 14% yield

### PREPARATION OF OXAZOLE ACIDS (as triethyl ammonium salts)

5-(4-fluorophenyl)-oxazole-2-carboxylic acid triethyl ammonium salt (OA-d):

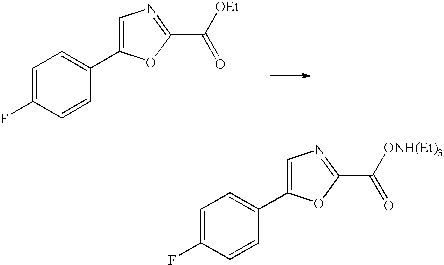

5-(4-fluorophenyl)-oxazole-2-carboxylic acid ethyl ester (3.56 g, 15.1 mmol) was dissolved in 140 mL of THF/H₂O (2.5:1). To this solution was added 3.3 g (5.2 eq) of LiOH H₂O. The resulting solution was stirred at ambient temperature overnight. The reaction mixture was adsorbed onto 18 g of silica gel and placed on a pad of silica gel. The triethyl ammonium salt was eluted off the pad with 200 mL of 10% MeOH/10% Et₃N/80% CH₂Cl₂. After concentrating the filtrate, 4.4 g of a brown oil was obtained (93% yield)

¹H NMR (300 MHz, DMSO) 6 7.78 (m, 2 H), 7.60 (s, 1H), 7.35 (m, 2 H), 3.15 (q, 6 H), 1.18 (t, 9 H). MS (APCI⁺) m/z 208(M+1).

5-(4-methoxynhenyl)-oxazole-2-carboxylic acid triethyl ammonium salt (OA-b):

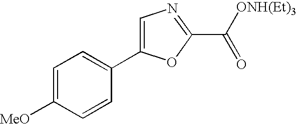

¹H NMR (300 MHz, CDCl₃) δ 7.7 (d, 2H), 7.3 (s, 1H), 6.9 (d, 2H), 3.9 (s, 3H), 3.2 (q, 6H), 1.4 (t, 9H). MS (APCI) rm/z 220 [M (free acid) +1].

Yield: 94%

5-(4-Methoxyphenyl)-oxazole-2-carboxylic acid triethyl ammonium salt (OA-i):

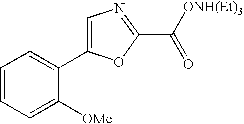

¹H NMR (300 MHz, CDCl₃) δ 8.00 (d, 1H), 7.65 (s, 1H), 7.30 (t, 1H), 6.95 (mn, 2H), 3.97 (s, 3H), 3.15 (q, 6H), 1.35 (t, 9H). MS (APCI⁺) m/z 220[M (free acid) +1]. Yield: 99%

### PREPARATION OF ANTHRANILATES

tert-Butyl 2-nitrobenzoate:

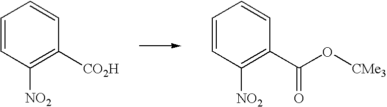

To a mixture of 2-nitrobenzoic acid (80.2 g, 0.48 mol) in DCM (2 L) was added DMF (4 mL) followed by the dropwise (55 min) addition of a solution of oxalyl chloride (100 μL, 1.15 mol) in DCM (0.4 L). After stirring at RT for 6 h, the reaction mixture was evaporated to dryness using heptane to aid in the chasing off of residual oxalyl chloride. This process was repeated and the two residue acid chlorides combined. To a stirred and cooled (0° C) solution of the combined acid chloride in dry THF (2 L) was added a solution of potassium tert-butoxide (130 g, 1.16 mol) in dry THF (1.4 L). The reaction mixture was stirred (I h), concentrated to one-fifth the original volume, dissolved in TBME (2 L) and then washed with 1 N NaOH (4×1.5 L), dried (Na₂SO₄), and evaporated to dryness (rotovap, then high vacuum) to give 194.8 g (yield of 91%) of the tert-butyl ester as a brown oil.

¹H NMR (300 MHz, CDCl₃) δ 7.86 (dd, 1H), 7.73 (dd, 1H), 7.55-7.70 (m, 2 H), 1.58 (s, 9 H).

tert-Butyl 2-aminobenzoate:

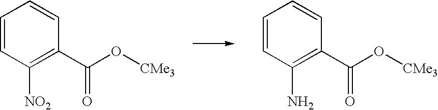

To a mixture of tert-butyl 2-nitrobenzoate (194.8 g, 0.873 mol) in EtOH (2.5 L) in a 5 L Parr bottle was added 72 g of Raney Nickel 2800 (washed 5 times with ethanol). The system was degassed (suction) three times, each time replenishing the system with nitrogen gas. The system was then pressurized with hydrogen gas (30 psi) and 1.5 h later was repressurized to 50 psi of hydrogen. After overnight stirring, the pressure of the system had fal]en to 30 psi and was repressurized to 50 psi. At the end of the day, the reaction was found to be complete. The reaction mixture was filtered thru Celite and evaporated to give 160.8 g (yield of 95%) of the aniline ester as a brown oil.

¹H NMR (300 MHz, CDCl₃) δ 7.82 (dd, 1H), 7.22 (ddd, 1H), 6.55-6.65 (m, 2 H), 5.7 (br s), 1.58 (s, 9 H).

tert-Butyl 2-amino-5-bromobenzoate:

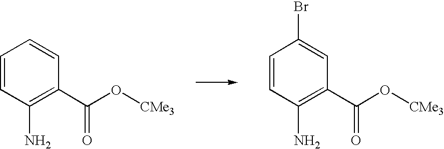

To a stirred and cooled (0° C.) solution of tert-butyl 2-aminobenzoate (23.37 g, 120.9 mmol) in DMF (20 mL) was added a mixture of NBS (21.50 g, 120.8 mmol) in DMF (20 mL) over a period of 3-5 min (an additional 10 mL of DMT used to rinse the NBS container was added as wel]). After stirring for 4 h (0° C. to RT), the reaction mixture was diluted with EtOAc (500 mL) and then washed with 1 N NaOH (3×300 mL), dried (Na₂SO4), filtered, evaporated on to silica gel (48 g), and applied to a column of silica gel (620 g) packed in 80:1 heptane/TBME. Elution was effected with 80:1, 70:1, and then 60:1 heptane/TBME to give (after evaporation of fractions) 25.99 g (yield of 79%) of the title product as a yellowish-white solid.

¹H NMR (300 MHz, CDCl₃) δ 7.91 (d, 1H), 7.30 (dd, 1H), 6.53 (d, 1H), 5.72 (br s), 1.58 (s, 9 H).

tert-Butyl 2-amino-5-iodobenzoate:

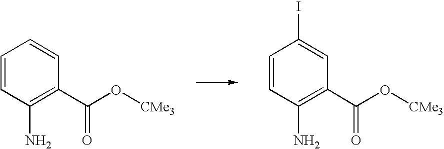

To a stirred and cooled (0° C.) solution of tert-butyl 2-aminobenzoate (71.3 g, 369 mmol) in DMF (25 mL) was added (dropwise) a mixture of NIS (87.0 g, 387 mmol) in diluted with EtOAc (1500 mL), washed with 1 N NaOH (3×500 mL), dried (Na₂SO₄), filtered, evaporated on to silica gel (150 g), and applied to a column of silica gel (1500 g) packed in 80:1 heptane/TBME. Elution was effected with 80:1, 70:1, and then 60:1 heptane/ TBME to give (after evaporation of desired fractions) 72.8 g (yield of 62%) of the title product as a solid.

¹H NMR (300 MHz, CDCl₃) δ 8.06 (d, 1H), 7.44 (dd, 1H), 6.43 (d, 1H), 5.75 (br s), 1.58 (s, 9 H).

tert-Butyl 2-amino-5-cyanobenzoate:

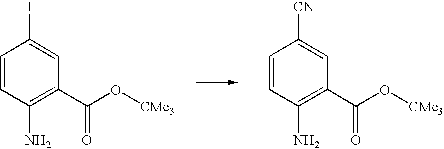

To a stirred and hot (internal temperature of 80-90° C.) solution of tert-butyl 2-amino-5-iodobenzoate (60.4 g, 189 mmol) in pyridine (125 mL) was added CuCN (20.5 g, 229 mmol). The resulting reaction mixture was then stirred at a gentle reflux (internal temperature of 120° C.) for 16 h, then allowed to cool to RT and diluted with EtOAc (150 mL) and heptane (25 μL). The copper salts were filtered off and the filtrate was then evaporated on to silica gel (80 g) and applied to a column of silica gel (1.2 Kg) packed in 8:1 heptane/ EtOAc. Elution was effected beginning with 8:1 and ending with 4:1 heptane/EtOAc. Evaporation of fractions gave 36.1 g (yield of 87%) of cyanoanthranilate as a light yellow fluffy solid.

¹H NMR (300 MHz, CDCl₃) δ 8.13 (d, 1H), 7.42 (dd, 1H), 6.65 (d, 1H), 6.8 (very br s), 1.60 (s, 9 H).

Methyl 2-Amino-5-cyanobenzoate (methyl

5-cyanoanthranilate):

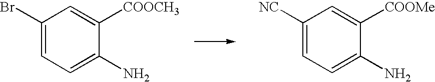

Methyl 2-amino-5-bromobenzoate (24.0 g, 104 mmol), zinc cyanide (8.02 g, 68.3 mmol), and Pd(PPh₃)₄ (2.02 g) were weighed into a three-necked flask, which was then purged with nitrogen. After adding DMF (130 mL), the reaction mixture was stirred in a hot oil bath (90° C.) for 8 h. Because TLC revealed no change during the last 3 h, additional catalyst (2.00 g) was added and stirred in the hot oil bath overnight. Additional catalyst (1.00 g) and heating at 100° C. was required to drive reaction to near completion. Reaction was poured into water (400 mL), extracted with EtOAc (700 mL), and the two-phase mixture filtered. The organic layer was removed, washed with saturated brine (4×200 mL), dried, filtered, and evaporated on to silica gel, and applied to a column of silica gel. Elution was effected using 4:1 heptane/EtOAc to give 15.60 g (yield of 85%) of the methyl

5-cyanoanthranilate as a pale yellow solid.

¹H NMR (300 MHz, CDCl₃) δ 8.19 (d, 1), 7.45 (dd, 1H), 6.68 (d, 1H), 6.3 (very brs),3.90(s, 3H).

2-Amino-5-cyanobenz ic acid (

5-cyanoanthranilic acid):

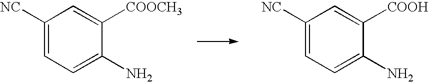

Methyl 2-amino-5-cyanobenzoate (5.0 g, 28.41 mmol) was dissolved in MeOH (105 mL). Then 6M NaOH (14 mL) was added. The resulting mixture was stirred at RT O/N. The reaction mixture was diluted with H₂O (143 mL), washed with DCM to remove any unreacted S.M. The product was precipitated from aqueous layer by addition of conc. HCl to pH 1 and extracted with EtOAc (3×100 mL). The combined organic layers were dried over MgSO₄. Solid was removed by filtration, and filtrate was concentrated to dryness under reduced pressure. The light yellow product was obtained in the yield of 94%.

¹H NMR (300 MHz, DMSO-d₆) δ 8.08 (d, 1H), 7.56 (dd, 1H), 7.50 (br), 6.85 (d, 1H).

Method B—Through a two step process:

(1) Coupling Of An Oxazole Acid Chloride With A Tert-Butyl Anthranilate

t-Butyl {

5-cyano-2-[5-(4-fluorophenyl)-oxazole-2-carbonyl]amino}benzoate (PRE(CN)-d):

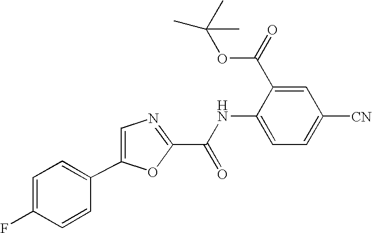

5-(4-fluorophenyl)-oxazole-2-carboxylic acid triethyl amrnmonium salt (1 g, 3.25 mmol) was dissolved in 25 mL of CH₂Ck₂. To this solution was added 3 drops of DMF, followed by 1 mL (3.5 eq) of oxalyl chloride dropwise. The resultant brown/orange solution was stirred at ambient temperature overnight. The solvent and excess oxalyl chloride were removed in vacuo to give an orange solid. The solid was dissolved in 12.5 mL of CH₂Cl₂ and added dropwise to a solution of 2-amino-5-cyano-benzoic acid (0.65 g, 0.94 eq), pyridine (5 mL) in 12.5 mL of CH₂Cl₂. The reaction was stirred at ambient temperature for 5 h. The reaction mixtue was diluted with 25 mL of TBME and washed with 50 mL of 1 N HCl, followed by 50 mL of brine solution. The organic layer was dried (MgSO₄), filtered, and the solvent removed in vacuo. The resulting solid was dissolved in 20 mL of MeOH and CH₂Cl₂ added until precipitation occurred (15 mL). The solution was placed in the freezer overnight to aid in crystal formation. The white solid was collected by filtration to give 1.38 g (36% yield).

¹H NMR (300 MHz, DMSO-d₆) δ 8.84 (d, 1H), 8.39 (d, 1H), 8.15 (d, 1H), 8.10 (s, 1H), 7.95 (m, 2H), 7.42 (m, 2H), 2.05 (s, 9 H).

t-Butyl {5-cyano-2-[5-(3-methyoxyvhenyl)-oxazole-2-carbonyl]amino}benzoate (PRE(CN)-a):

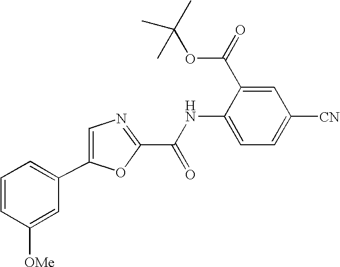

¹H NMR (300 MHz, CDCl₃) δ 12.59 (br), 8.78 (d, 1H), 8.40 (s, 1H), 8.20 (dd, 1 H), 8.17 (s, 1H), 7.40 (m, 3H), 7.05 (m, 1H), 3.85 (s, 3H), 1.70 (s, 9H). Yield: 15

t-butyl{

5-cyano-2-[5-(4-methyoxyvhenyl)-oxazole-2-carbonyl]amino}benzoate (PRE(CN-b):

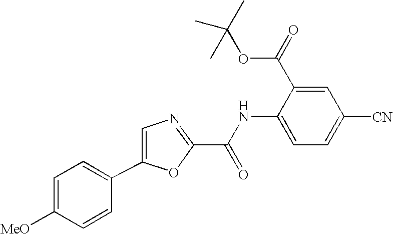

¹H NMR (300 MHz, CDCl₃) δ 12.82 (br), 9.05 (d, 1H), 8.35 (d, 1H), 7.85 (dd, 1H), 7.75 (dd, 2H), 7.45 (s, 1H), 7.00 (d, 2H), 3.90 (s, 3H), 1.70 (s, 9H).

Yield: 41%

t-Butyl {

5-cyano-2-[5-(2-methyoxyvhenyl)-oxazole-2-carbonyl]amino}benzoate (PRE(CN)-i):

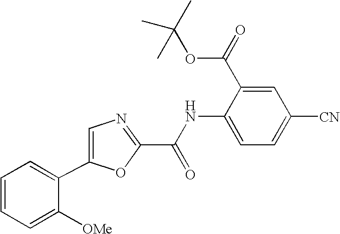

¹H NMR (300 MHz, CDCl₃) δ 12.89 (br), 9.05 (d, 1H), 8.35 (s, 1H), 8.03 (dd, 1H), 7.85 (dd, 1H), 7.80 (s, 1H), 7.40 (m, 1H), 7.10 (t, 1H), 7.00 (d, 1H), 4.00 (s, 3H), 1.70 (s, 9H).

MS (APCI⁺) m/z 420(M+1).

Yield: 55%

(2) Deprotection of the Tert-Butyl Ester

5-Cyano-2-{[5-(4-fluoronhenyl)-oxazole-2-carbonyl]amino}benzoic acid (TI(CN)-d):

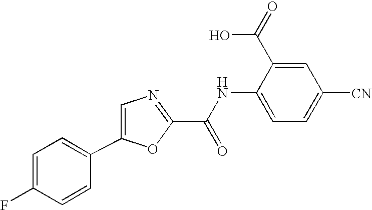

t-Butyl {

5-cyano-2-[5-(4-fluorophenyl)-oxazole-2-carbonyl]amino}benzoate (480 mg, 1.1 mmol) was dissolved in 22 mL of CH₂Cl₂. To this solution was added 7 mL of trifluoroacetic acid. The resultant mixture was stirred at room temperature overnight, followed by 40° C. (oil bath temperature) for 3 h. The solvent was removed in vacuo to give 420 mg of a light brown solid. This solid was heated in 50 mL of MeOH to boiling in a 2 L emlenmeyer flask. Dichloromethane was added dropwise to the solution (maintaing the boil) until the solid had mostly dissolved. The solution was filtered hot to remove the insouble impurities. The clear filtrate was then heated until the solution became quite cloudy. The solution was cooled to room temperature and the placed in the freezer overnight. The white solid that formed was collected by filtration to give 230 mg (57% yield).

¹H NMR (300 MHz, DMSO-d₆) δ 13.50 (br), 8.82 (d, I1H), 8.41 (s, 1H), 8.13 (dd, 1H), 8.05 (s, 1H), 7.92 (m,22H), 7.42 (m 2H).

5-Cyano-2-{I5-(3-methoxyphenyl)-oxazole-2-carbonyl]amino}benzoic acid (TI(CN)-a):

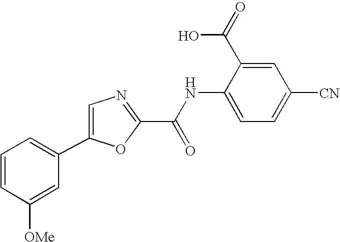

¹H NMR (300 MHz, DMSO-d₆) δ 12.95 (br), 8.85 (d, 1H), 8.43 (s, 1H), 8.14 (d, 1H), 8.09 (s, 1H), 7.45 (m, 3H), 7.04 (m, 1H), 3.84 (s, 3H). (TI(CN)-b):

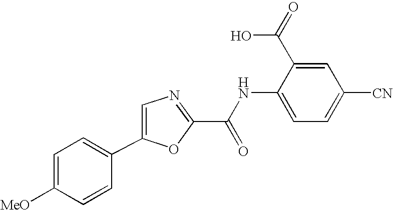

¹H NMR (300 MHz, DMSO-d₆) δ 13.1 (br), 8.83 (d, 1H), 8.45 (d, 1H), 8.12 (dd, 1H), 7.92 (s, 1H), 7.80 (d, 2H), 7.10 (d, 2H), 3.86 (s, 3H).

5-Cyano-2-f [5-(2-methyoxyphenyl)-oxazole-2-carbonyl]amino]benzoic acid (TI(CN-i):

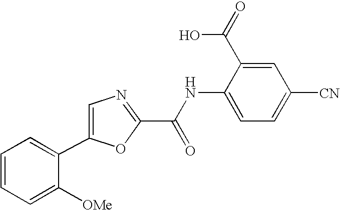

¹H NMR (300 MHz, DMSO-d₆) δ 12.95 (br), 8.85 (d, 1H), 8.45 (s, 1H), 8.13 (dd, 1H), 7.90 (s, 1H), 7.85 (d, 1H), 7.45 (n, 1H), 7.25 (d, 1H), 7.15 (t, 1H), 4.00 (s, 3H).

5-Bromo-2- f [5-(4-fluorophenyl)-oxazole-2-carbonyl]amino]benzoic acid (TI(Br)-d)

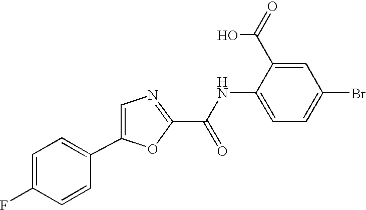

¹H NMR (300 MHz, DMSO-d) δ 12.60 (br), 8.69 (d, 1H), 8.14 (d, 1H), 8.05 (s, 1H), 7.92 (m, 3 H), 7.42 (m, 2H).

Method B—Direct Coupling Of Oxazole Esters With Anthranilic Acids

5-cyano-2-{[5-(2-furanyl)-oxazole-2-carbonyl]amino}benzoic acid (TI(CN)-h):

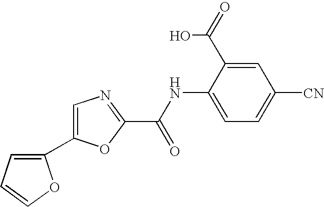

To a solution of 2-amino-5-cyano-benzoic acid (0.389 g, 2.4 mmmol) and oxazole ester (1 eq) in DMF (5 mL) was added NaH (3 eq). The solution was allowed to stir at room temperature overnight. The reaction mixture was added dropwise to 100 mL of 1 N HCl with stirring. The carboxylic acid precipitated out and was collected by filtration. The tan solid was recrystallized from boiling EtOH to give 230 mg of an off white solid (yield of 32%).

¹H NMR (300 MHz, DMSO-d₆) δ 12.91 (bs), 8.86 (d, 1H), 8.45 (s, 1H), 8.18 (dd, 1H), 7.98 (s, 1H), 7.84 (s, 1H), 7.12 (d, 1H), 6.78 (d, 1H).

5-Cyano-2-[(5-nIhenyl-oxazole-2-carbonyl)amino]benzoic acid (TI(CN)—I):

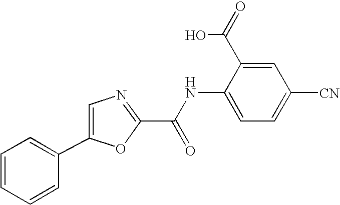

¹H NMR (300 MHz, DMSO-d₆) δ 12.71 (br), 8.84 (d, 1H), 8.41 (s, 1H), 8.14 (dd, 1H), 8.10 (s, 1H), 7.88 (d, 2H), 7.50 (m, 3H).

5-Cyano-2-15-(2-trifluoromethylphenyl)-oxazole-2-carbonyil amino]benzoic acid (TI(CN)-g:

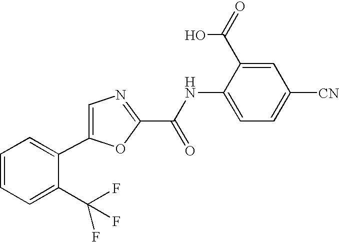

¹H NMR (300 MHz, DMSO-d₆) δ 12.71 (br), 8.82 (d, 1H), 8.41 (s, 1H), 8.14 (m, 3H), 7.88 (m, 2H), 7.74 (t, 1H).

5-Cyano-2- {[5-(3.4-methylenedioxyphenyl)-oxazole-2-carbonyl]amino}benzoic acid (TI(CN)-k):

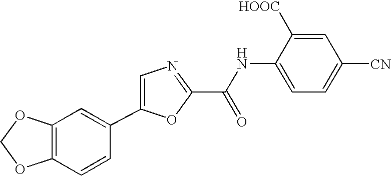

¹H NMR (300 MHz, DMSO-d₆) δ 12.95 (br), 8.87 (d, 1H), 8.45 (d, 1H), 8.13 (dd, 1H), 7.95 (s, 1H), 7.40-7.47 (m, 2H), 7.10 (d, I1H), 6.17 (s, 2H).

5-Cyano-2-f [5-(4-cyanoyhenyl)-oxazole-2-carbonvyl amino]benzoic acid (TI(CN)-l):

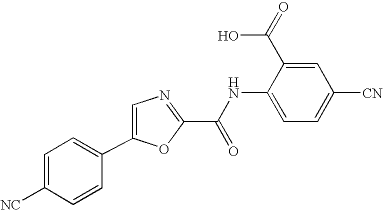

¹H NMR (300 MHz, DMSO-d₆) δ 13.00 (br), 8.82 (d, 1H), 8.43 (d, 1H), 8.28 (s, 1H), 8.12 (dd, 1H), 8.04 (s, 4H).

5-Cyano-2-{[5-(3-cyanoiphenyl)-oxazole-2-carbonyl]amino}benzole acid (TI(CN)-m):

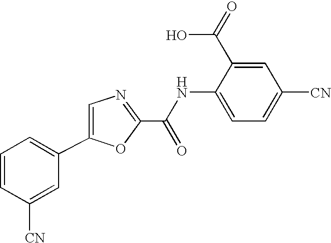

¹H NMR (300 MHz, DMSO-d₆) δ 12.96 (br), 8.82 (d, 1H), 8.37 (d, 2H), 8.25 (s, 1H), 8.12 (dd, 2H), 7.93 (d, 1H), 7.74 (t, 1H).

5-Cyano-2-{[5-(1-methyl-1H-pyrrol-2-yl)-oxazole-2-carbonyl]amino}benzoic acid (TI(CN)-s):

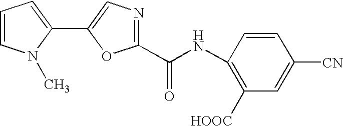

¹H NMR (300 MHz, DMSO-d₆) δ 12.65 (br), 8.85 (d, 1H), 8.41 (s, 1H), 8.19 (dd, 1H), 7.62 (s, 1H), 7.10 (s, 1H), 6.65 (dd, 1H), 6.18 (t, I1H).

5-Bromo-2-f [5-(4-methyoxyuhenyl)-oxazole-2-carbonvil amino]benzoic acid (TI(Br)-b):

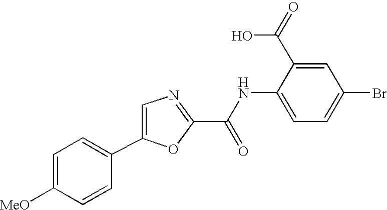

¹H NMR (300 MHz, DMSO-d₆) δ 12.60 (br), 8.65 (d, 1H), 8.20 (s, 1H), 7.90 (m, 2H), 7.80 (d, 2H), 7.10 (d, 2H), 3.80 (s, 3H).

5-Bromo-2-[(5-phenyl-oxazole-2-carbonyl)amino]benzoic acid (TI(Br)-f):

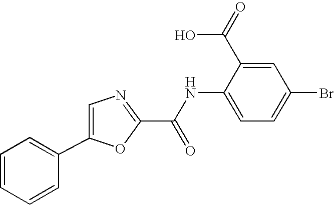

¹H NMR (300 MHz, DMSO-d₆) δ 12.65 (br), 8.65 (d, 1H), 8.19 (d, 1H), 8.10 (s, 1H), 7.90 (m, 3H), 7.50 (m, 3H).

5-Bromo-2-[[5-(2-trifluoromethylnhenyl)-oxazole-2-carbonyl]amino]benzoic acid (TI(Br)-g):

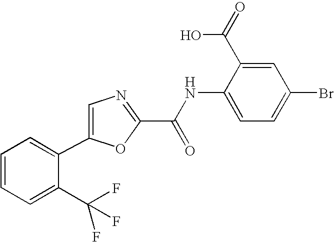

¹H NMR (300 MHz, DMSO-d₆) δ 12.67 (br), 8.65 (d, 1H), 8.13 (d, 1H), 7.76-7.98 (m, 6H).

5-Bromo-2-[[5-(2-furanyl)-oxazole-2-carbonyl]amino]benzoic acid (TI(Br)-h):

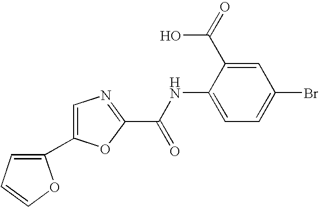

¹H NMR (300 MHz, DMSO-d₆) δ 8.67 (d, 1H), 8.16 (s, 1H), 7.97 (s, 1H), 7.92 (d, 1H), 7.81 (s, 1H), 7.09 (d, 1H), 6.77 (d, 1H).

5-Bromo-2-[[5-(2-methyoxyyhenyl)-oxazole-2-carbonyl]amino]benzoic acid (TI(Br)-i):

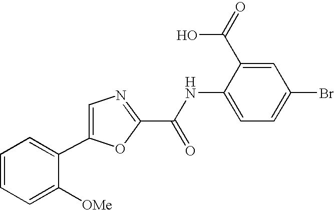

¹H NMR (300 MHz, DMSO-d₆) δ 12.65 (br), 8.64 (d, 1H), 8.13 (d, 1H), 7.84 (m, 3H), 7.44 (m, 1H), 7.10 (m, 2H), 3.98 (s, 3H).

5-Bromo-2-{[5-(3,4-methylenedioxyphenyl)-oxazole-2-carbonyl]amino}benzoic acid (TI(Br)-k):

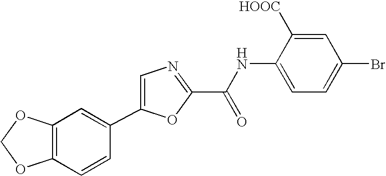

¹H NMR (300 MHz, DMSO-d₆) δ 12.63 (br), 8.63 (d, 1H), 8.15 (d, 1H), 7.89 (s, 1H), 7.85 (dd, 1H), 7.40 (d, 1H), 7.34 (dd, 1H), 7.10 ((d, 1H), 6.15 (s, 2H).

5-Bromo-2-{[5-(4-cyanophenyl)-oxazole-2-carbonyl]amino}benzoic acid (TI(Br)-l):

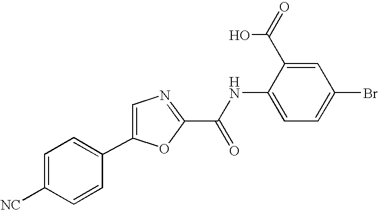

¹H NMR (300 MHz, DMSO-d₆) δ 12.68 (br), 8.62 (d, 1H), 8.24 (s, 1H), 8.12 (d, 1H), 8.01 (s, 4H), 7.86 (dd, 1H).

5-Bromo-2-{[5-(3-cyanophenyl)-oxazole-2-carbonyl]amino}benzoic acid (TI(Br)-m):

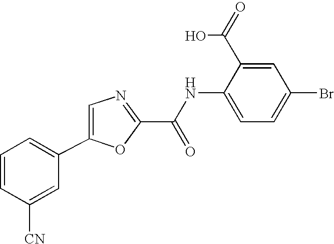

¹H NMR (300 MHz, DMSO-d₆) δ 12.71 (br), 8.64 (d, 1H), 8.37 (s, 1H), 8.14 (m, 3H), 7.88 (m, 2H), 7.74 (t, 1H).

5-Bromo-2-{[5-(1-methyl-1H-nvrrol-2-yl)-oxazole-2-carbonyl]amino}benzoic acid (TI(Br)-s):

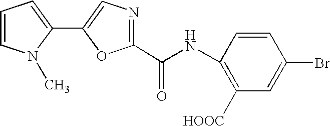

¹H NMR (300 MHz, DMSO-d) δ 12.59 (br), 8.65 (d, 1H), 8.14 (d, 1H), 7.88 (dd, 1H), 7.64 (s, 1H), 7.04 (d, 1H), 6.63 (dd, 1H), 6.17 (dd, 1H).

### Example 11: Activity Data

MIC Test Method

The in vitro MiCs of test compounds were determined by a standard agar dilution method. A stock drug solution of each analog was prepared in the preferred solvent, usually DMSO:H₂O (1:3). Serial 2-fold dilutions of each sample are made using 1.0 ml aliquots of sterile distil]ed water. To each 1.0 ml aliquot of drug was added 9 ml of molten Mueller Hinton agar medium. The drug-supplemented agar was mixed, poured into 15×100 mm petri dishes, and allowed to solidify and dry prior to inoculation.

Vials of each of the test organisms are maintained frozen in the vapor phase of a liquid nitrogen freezer. Test cultures are grown overnight at 35° C. on the medium appropriate for the organism. Colonies are harvested with a sterile swab, and cell suspensions are prepared in Trypticase Soy broth (TSB) to equal the turbidity of a 0.5 McFamland standard. A 1:20 dilution of each suspension was made in TSB. The plates containing the drug supplemented agar are inoculated with a 0.001 ml drop of the cell suspension using a Steers replicator, yielding approximately 10⁴ to 10⁵ cells per spot. The plates are incubated overnight at 35° C.

Following incubation the Minimum Inhibitory Concentration (MIC μg/ml), the lowest concentration of drug that inhibits visible growth of the organism, was read and recorded.

| | SAUR 9218 |
|---|---|
| COMPOUND | MIC |
| 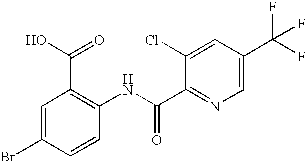 | 64 |
| 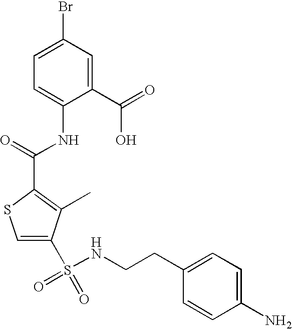 | 8 |
| 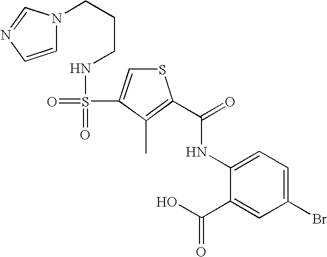 | >128 |
| 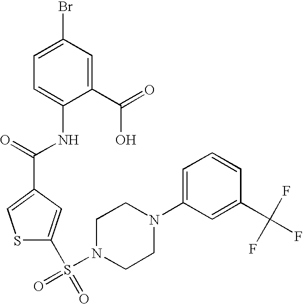 | 16 |
| 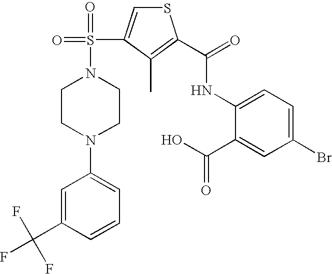 | 16 |
| 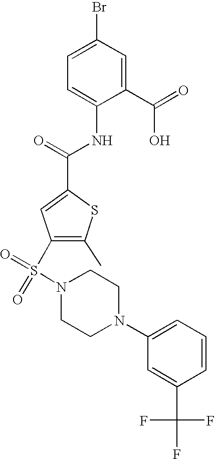 | 32 |
| 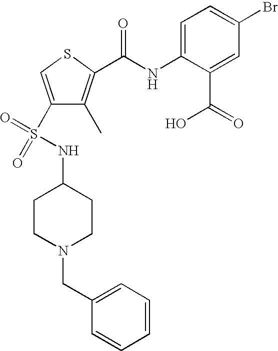 | >128 |
| 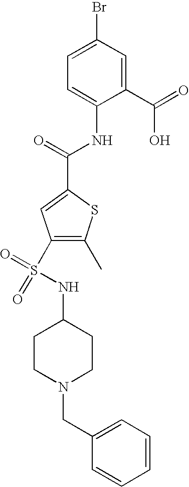 | >128 |
| 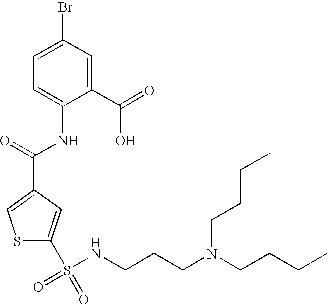 | >128 |
| 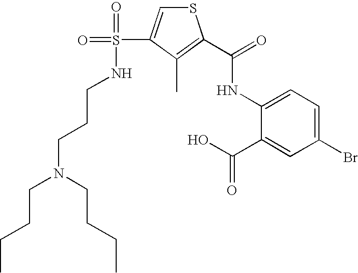 | >128 |
| 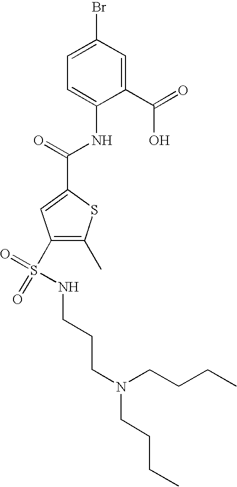 | >128 |
| 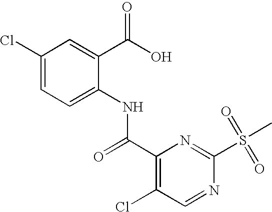 | >128 |
| 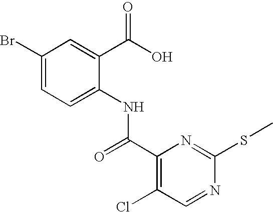 | >128 |
| 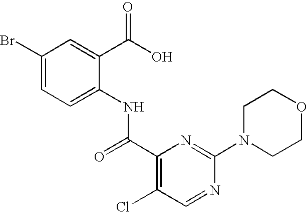 | >128 |
| 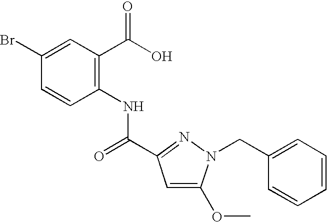 | 128 |
| 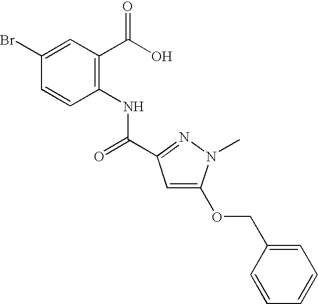 | 16 |
| 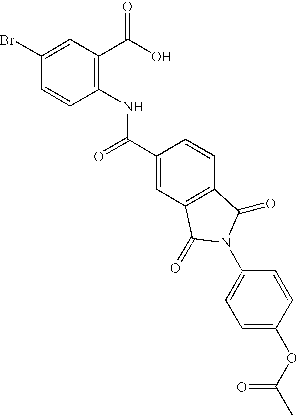 | 64 |
| 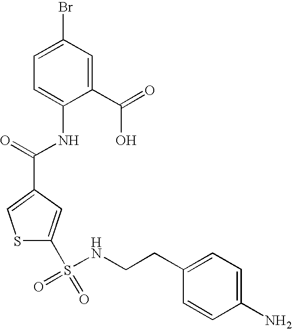 | 128 |
| 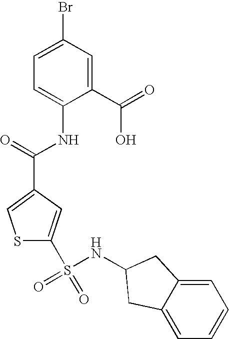 | 8 |
| 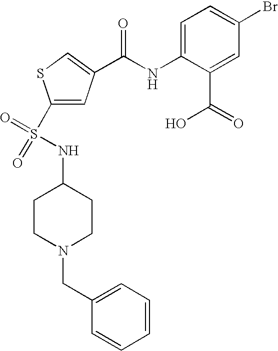 | 64 |
| 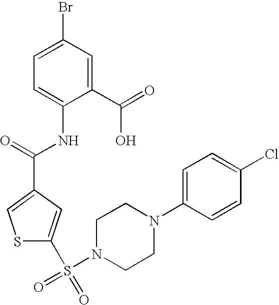 | 32 |
| 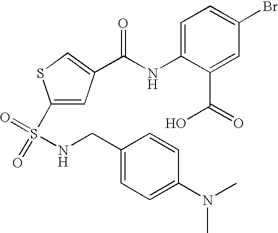 | 64 |
| 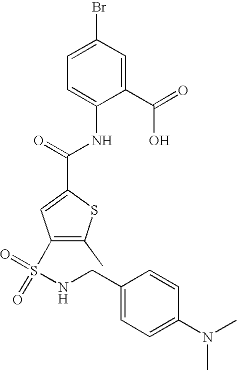 | 16 |
| 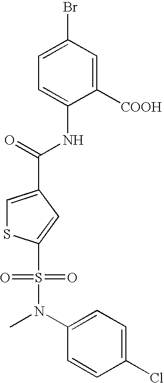 | 2 |
| 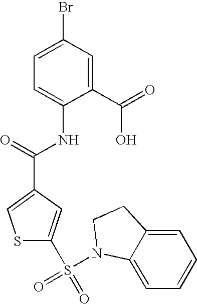 | 2 |
| 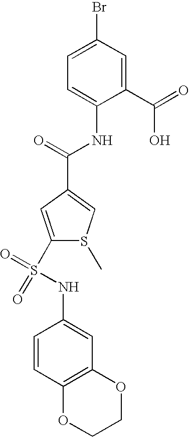 | 16 |
| 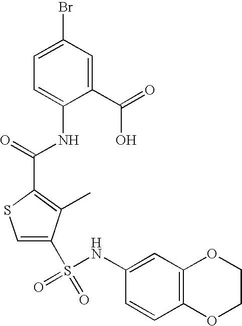 | 16 |
| 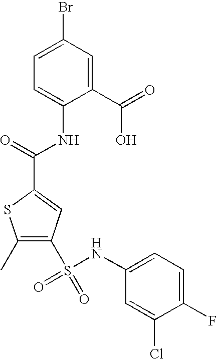 | 8 |
| 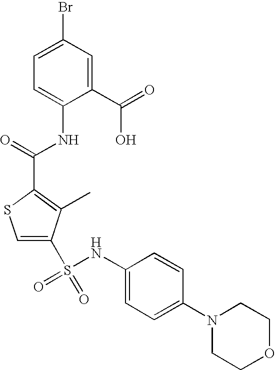 | 64 |
| 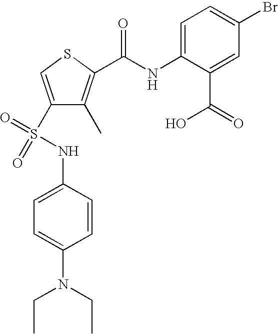 | 64 |
| 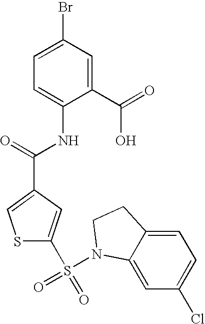 | 8 |
| 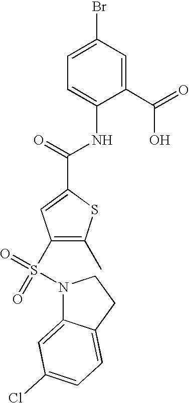 | 1 |
| 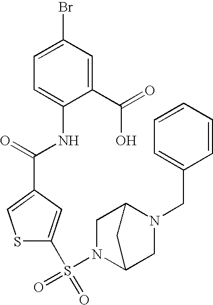 | >128 |
| 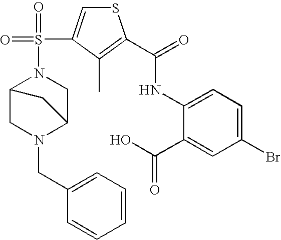 | >128 |
| 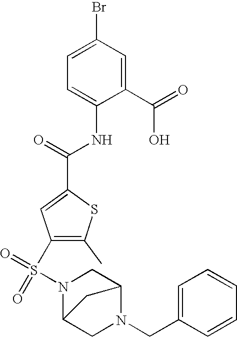 | 16 |
| 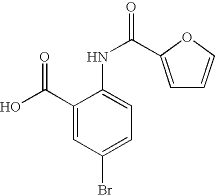 | >128 |
| 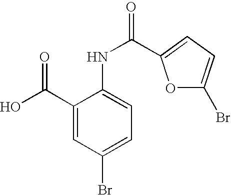 | 64 |
| 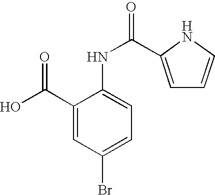 | >128 |
| 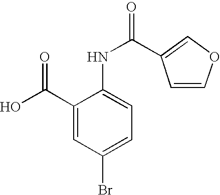 | >128 |
| 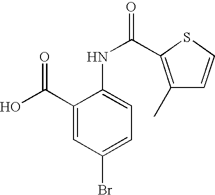 | >128 |
| 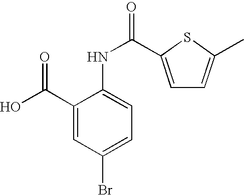 | 64 |
| 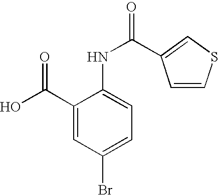 | >128 |
| 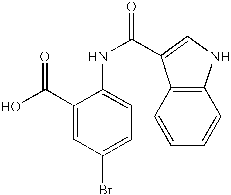 | 4 |
| 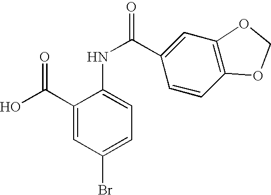 | 16 |
| 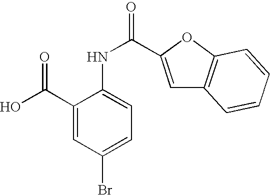 | 8 |
| 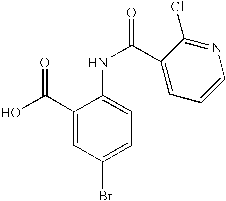 | >128 |
| 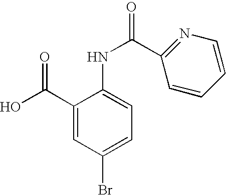 | >128 |
| 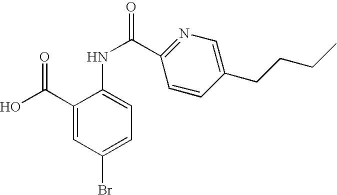 | >128 |
| 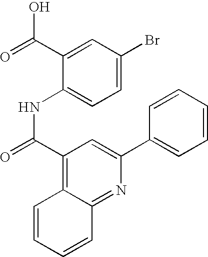 | 64 |
| 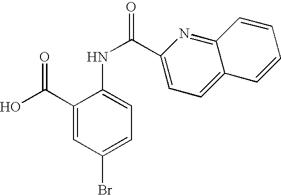 | 8 |
| 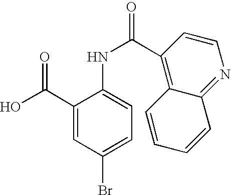 | >128 |
| 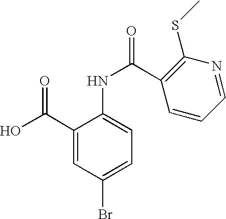 | >128 |
| 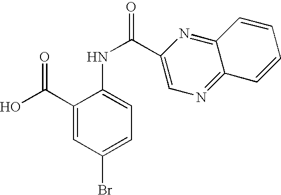 | 8 |
| 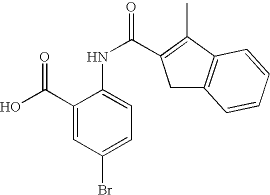 | 8 |
| 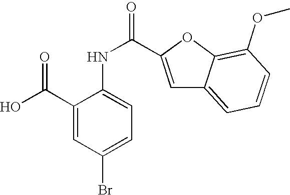 | 32 |
| 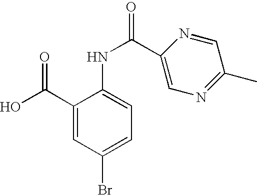 | 128 |
| 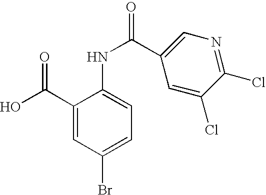 | 4 |
| 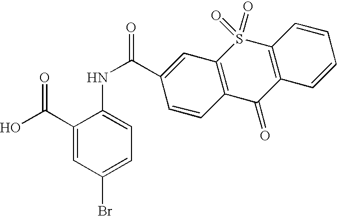 | 16 |
| 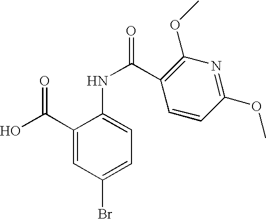 | >128 |
| 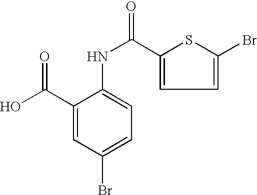 | 16 |
| 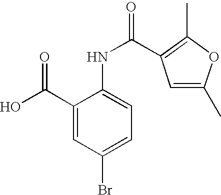 | >128 |
| 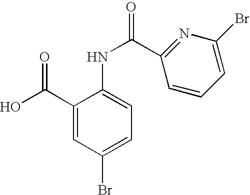 | 64 |
| 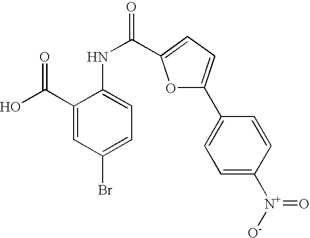 | 64 |
| 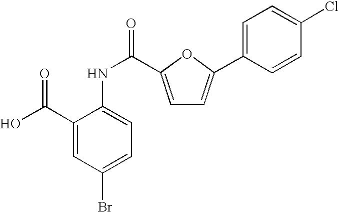 | 32 |
| 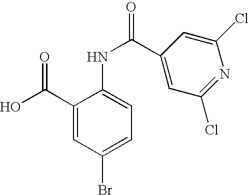 | 32 |
| 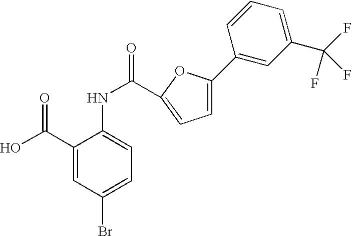 | 16 |
| 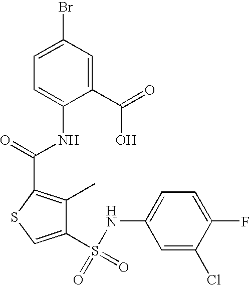 | 64 |
| 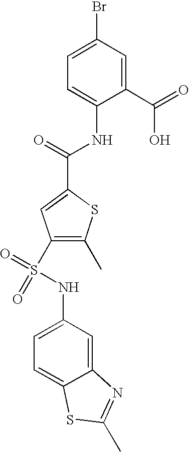 | 32 |
| 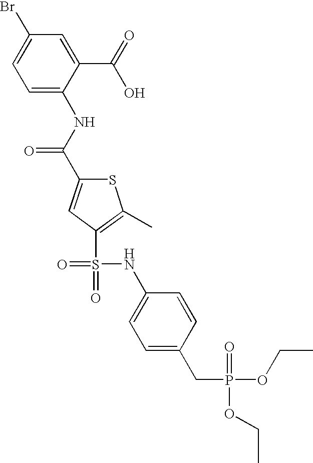 | 64 |
| 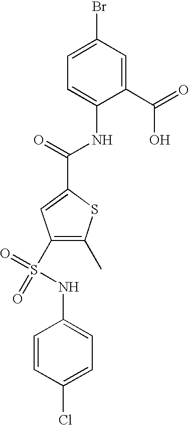 | 4 |
| 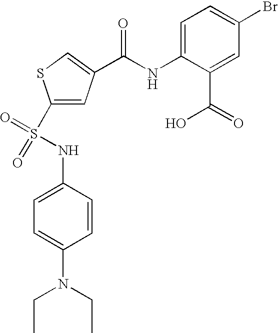 | >128 |
| 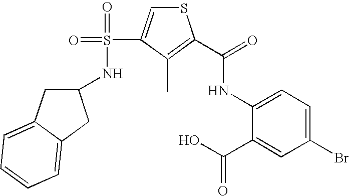 | 16 |
| 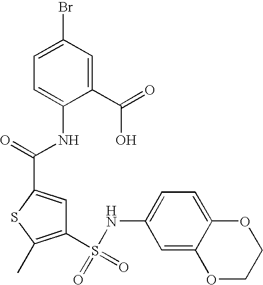 | 8 |
| 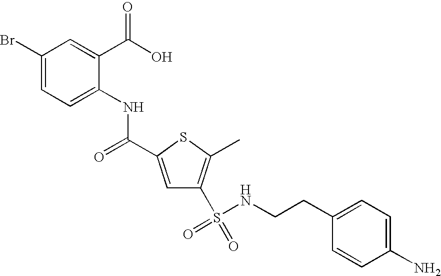 | 32 |
| 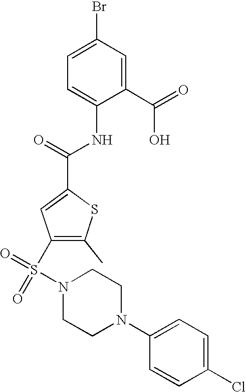 | >128 |
| 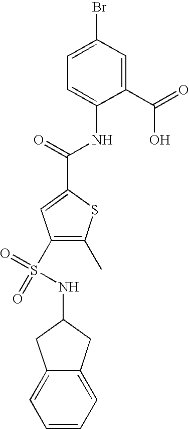 | 8 |
| 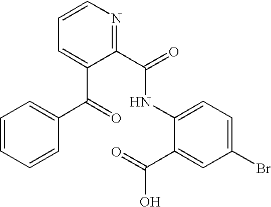 | >128 |
| 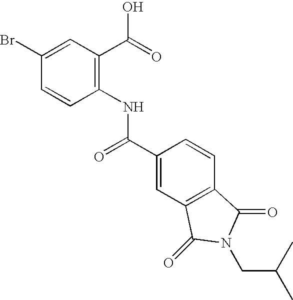 | 2 |
| 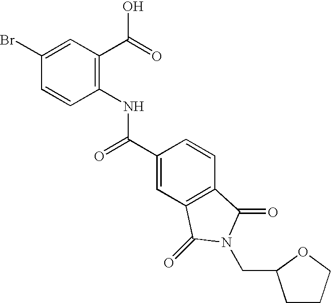 | 8 |
| 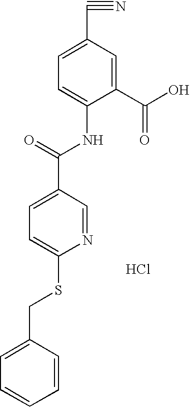 | 0.25 |
| 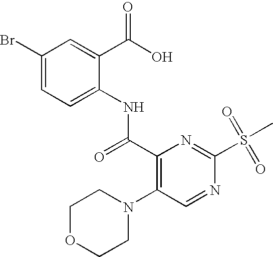 | >128 |
| 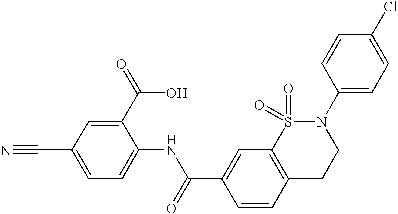 | 0.25 |
| 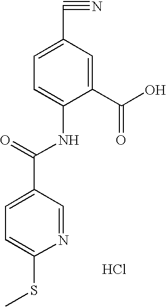 | 4 |
| 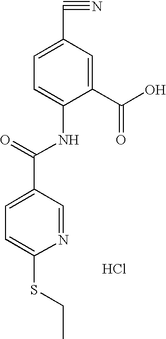 | 0.5 |
| 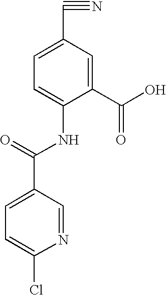 | 16 |
| 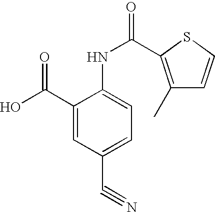 | 1 |
| 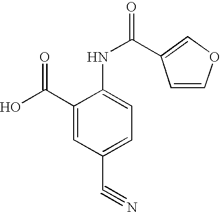 | >128 |
| 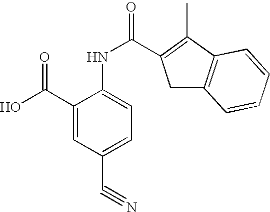 | 4 |
| 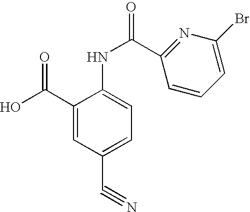 | 32 |
| 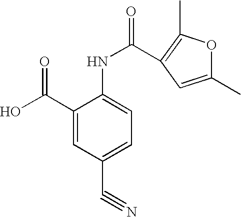 | >128 |
| 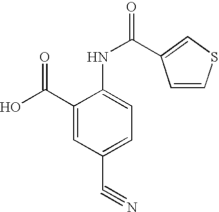 | >128 |
| 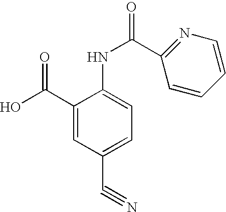 | 32 |
| 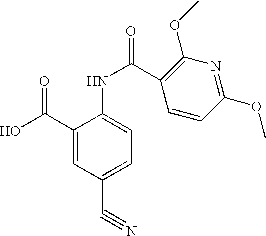 | >128 |
| 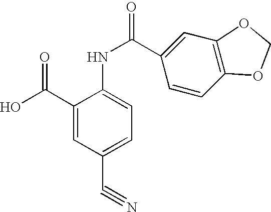 | 16 |
| 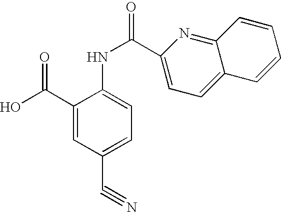 | 8 |
| 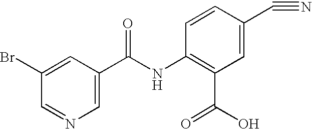 | 8 |
| 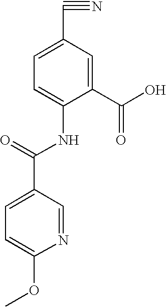 | 8 |
| 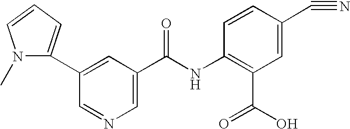 | 8 |
| 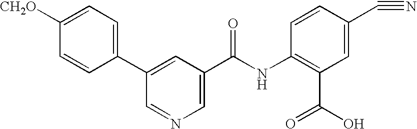 | 8 |
| 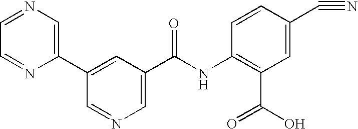 | 16 |
| 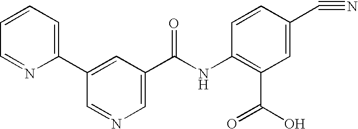 | >128 |
| 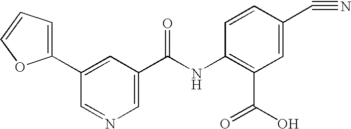 | 4 |
| 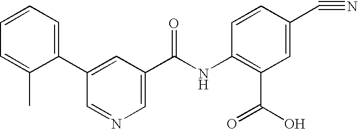 | 8 |
| 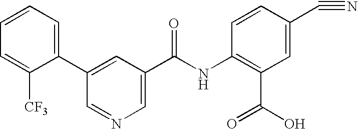 | 2 |
| 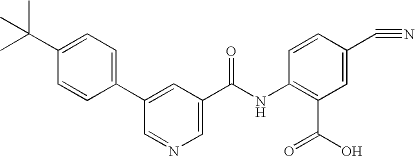 | 32 |
| 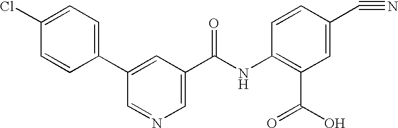 | >128 |
| 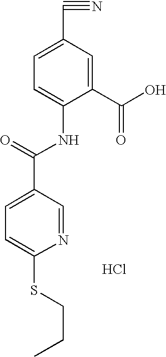 | 0.5 |
| 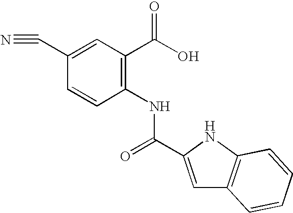 | 2 |
| 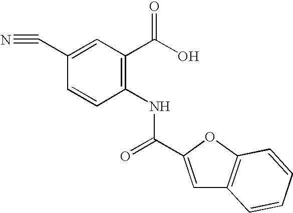 | 2 |
| 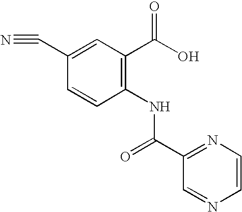 | 128 |
| 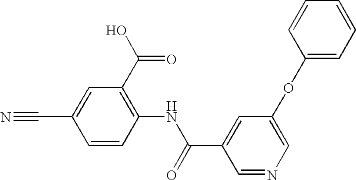 | 8 |
| 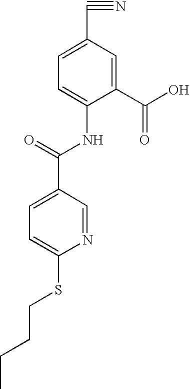 | 0.25 |
| 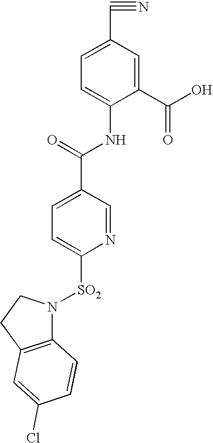 | 2 |
| 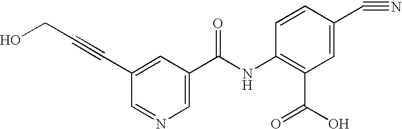 | 64 |
| 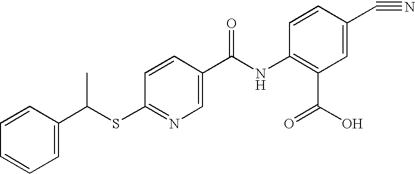 | 0.25 |
| 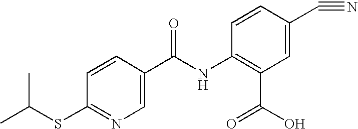 | 0.25 |
| 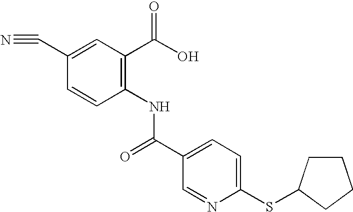 | 0.5 |
| 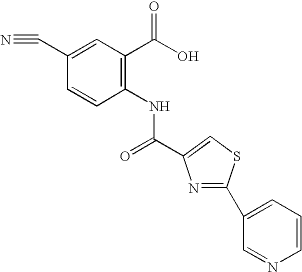 | >128 |
| 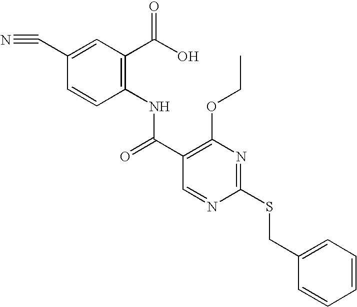 | 8 |
| 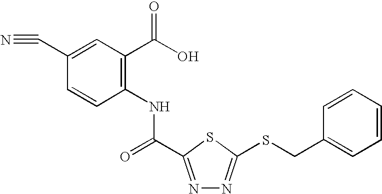 | 0.125 |
| 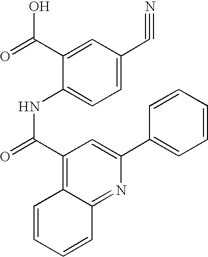 | 16 |
| 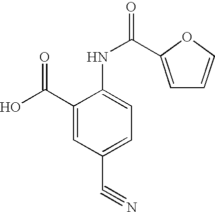 | >128 |
| 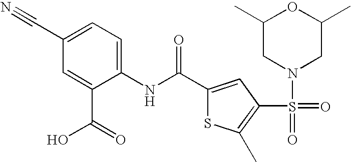 | 16 |
| 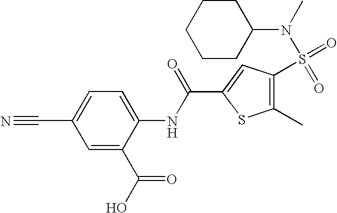 | 8 |
| 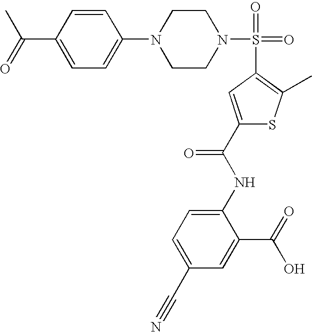 | 16 |
| 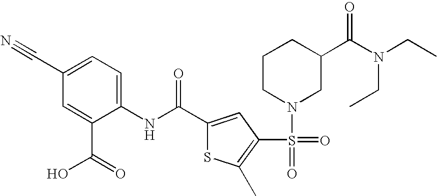 | 64 |
| 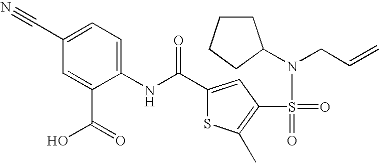 | 8 |
| 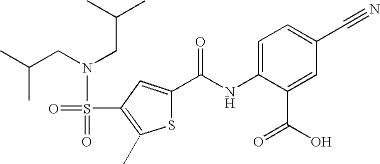 | 32 |
| 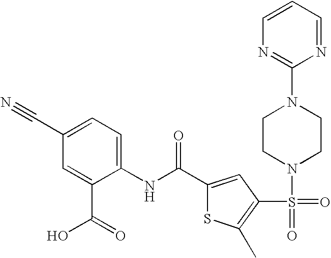 | 64 |
| 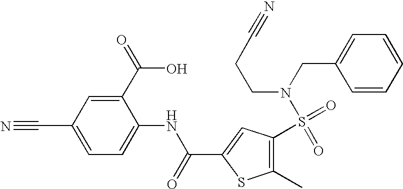 | 64 |
| 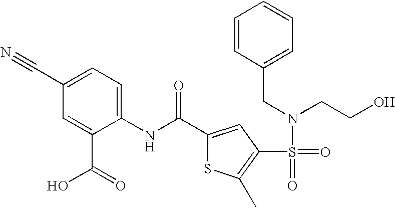 | 16 |
| 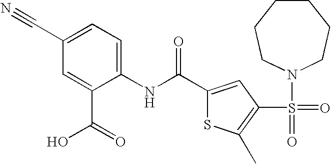 | 8 |
| 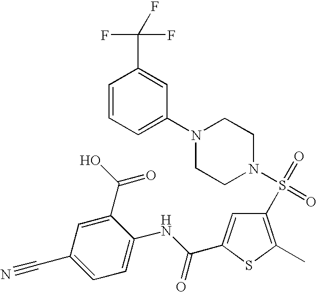 | 32 |
| 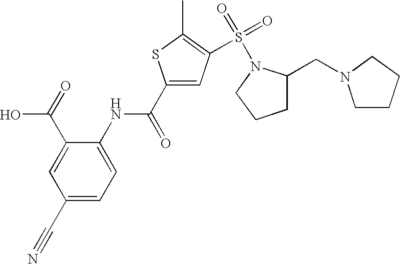 | 128 |
| 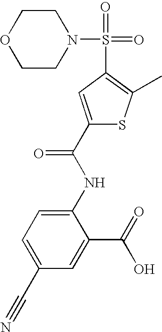 | 32 |
| 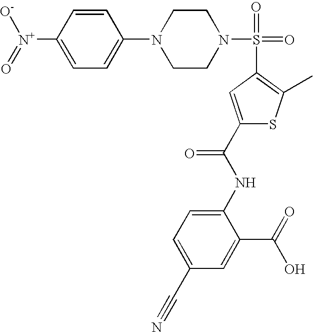 | 4 |
| 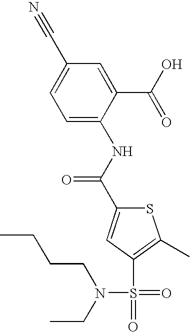 | 8 |
| 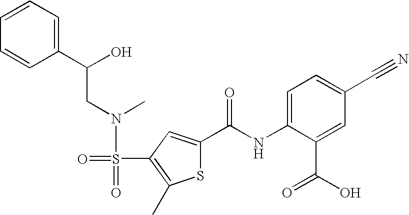 | 2 |
| 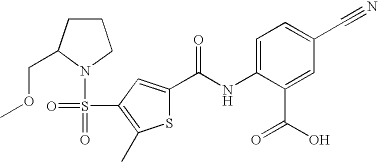 | 64 |
| 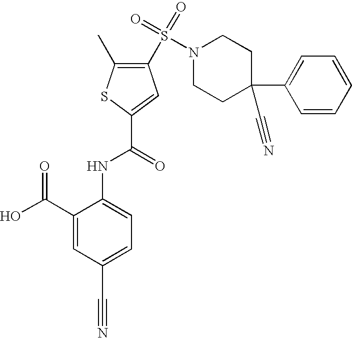 | 8 |
| 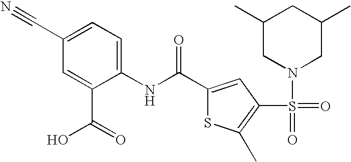 | 16 |
| 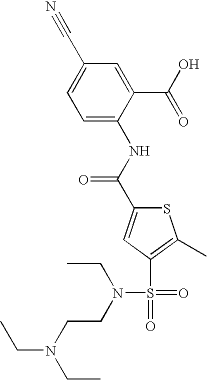 | 128 |
| 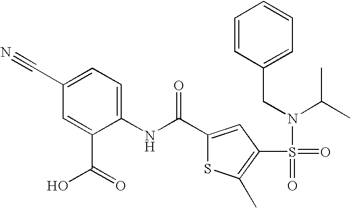 | 32 |
| 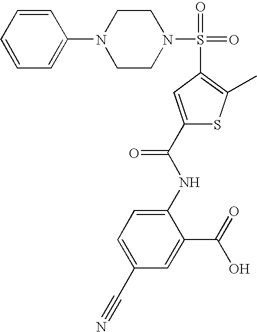 | 8 |
| 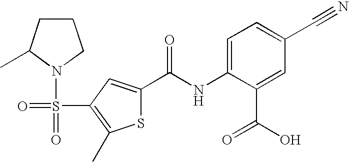 | 8 |
| 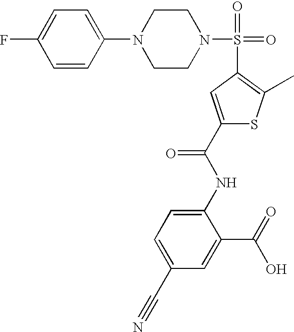 | 16 |
| 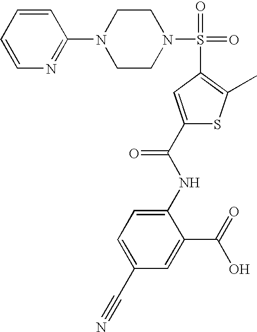 | 32 |
| 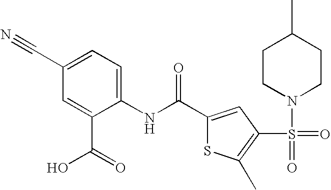 | 8 |
| 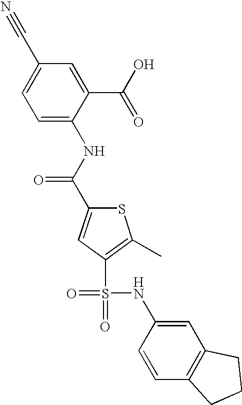 | 32 |
| 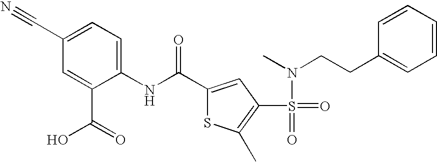 | 4 |
| 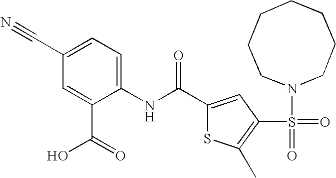 | 8 |
| 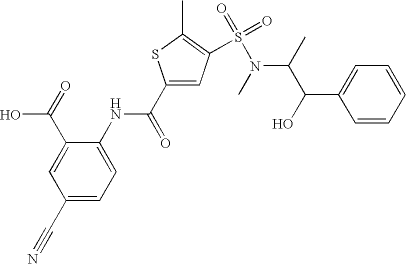 | 32 |
| 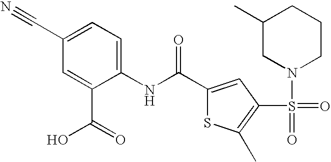 | 2 |
| 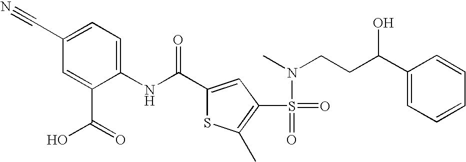 | 64 |
| 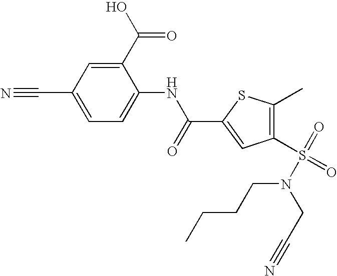 | 64 |
| 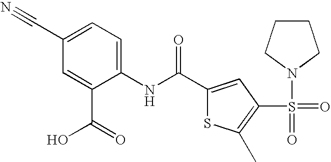 | 16 |
| 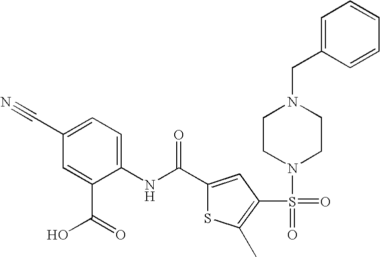 | >128 |
| 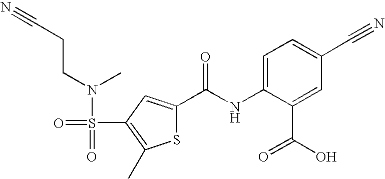 | 128 |
| 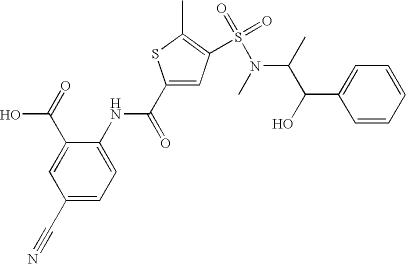 | 128 |
| 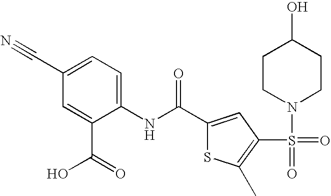 | 16 |
| 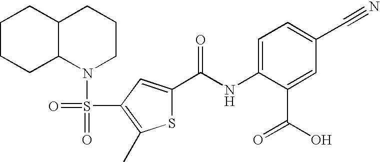 | 32 |
| 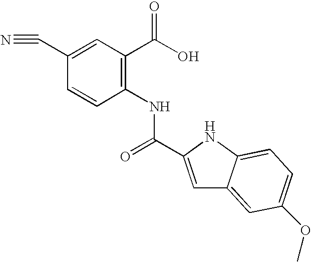 | 128 |
| 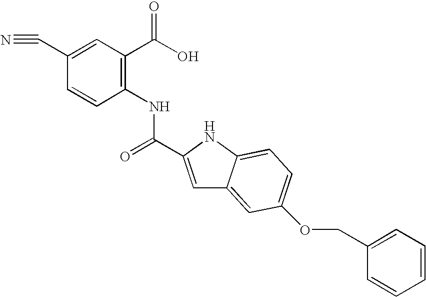 | 16 |
| 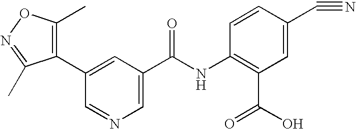 | 16 |
| 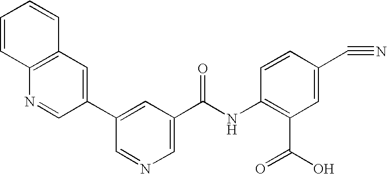 | >128 |
| 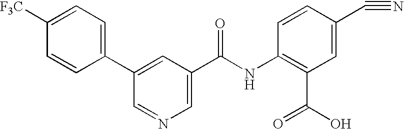 | >128 |
| 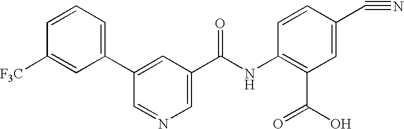 | 16 |
| 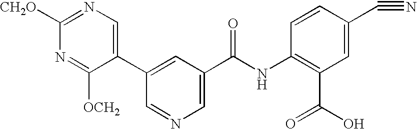 | >128 |
| 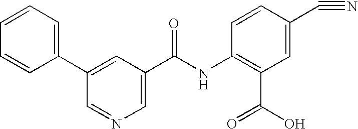 | 16 |
| 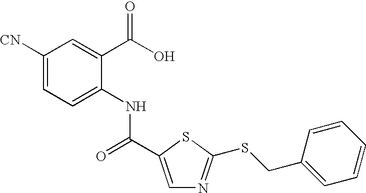 | 1 |
| 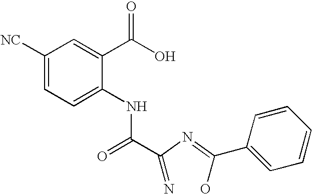 | 4 |
| 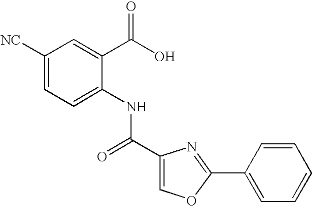 | 64 |
| 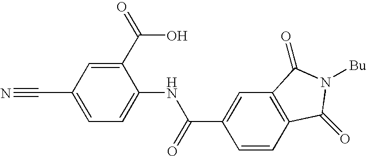 | 4 |
| 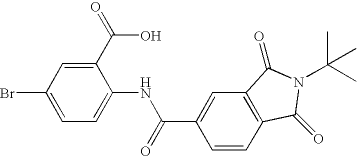 | 4 |
| 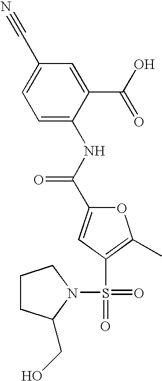 | 8 |
| 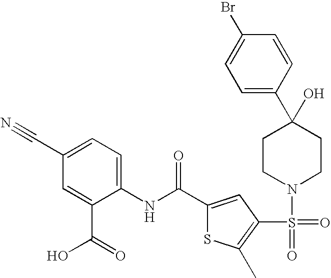 | 32 |
| 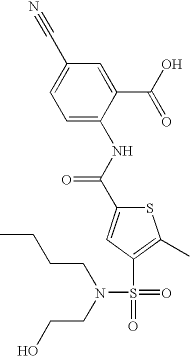 | 32 |
| 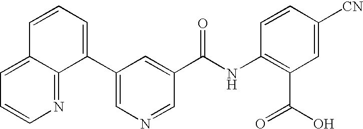 | 32 |
| 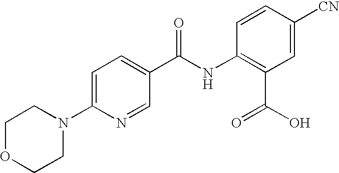 | 64 |
| 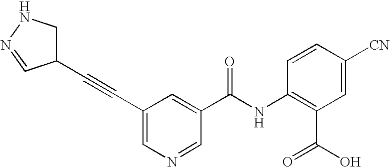 | 4 |
| 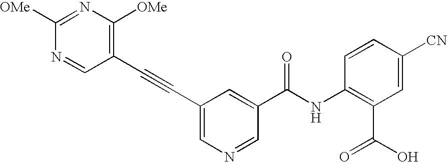 | >128 |
| 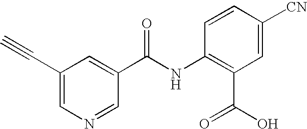 | 16 |
| 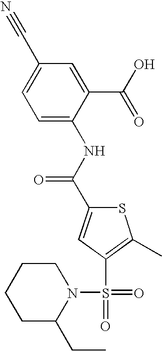 | 2 |
| 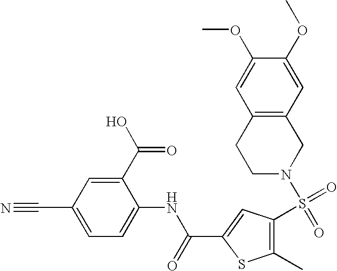 | 8 |
| 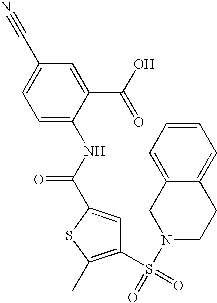 | 4 |
| 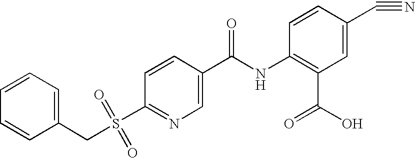 | 64 |
| 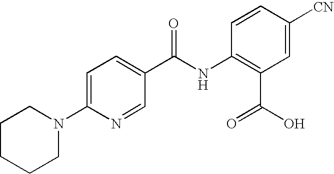 | 8 |
| 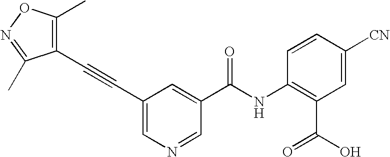 | 16 |
| 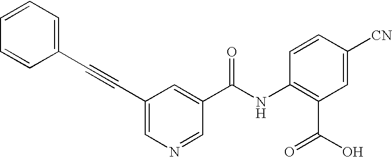 | 4 |
| 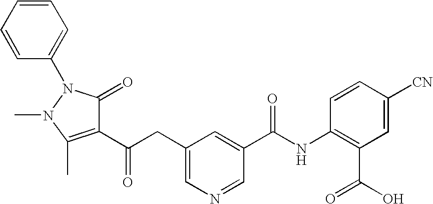 | 32 |
| 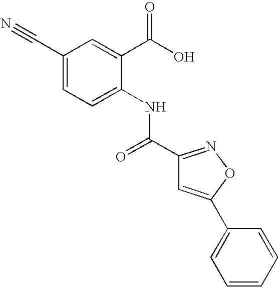 | 0.25 |
| 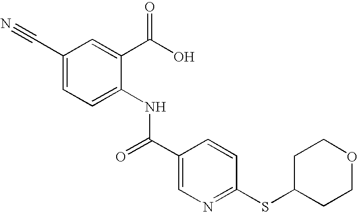 | 0.125 |
| 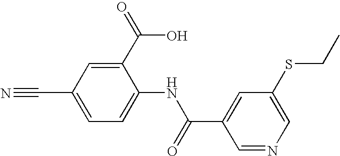 | 1 |
| 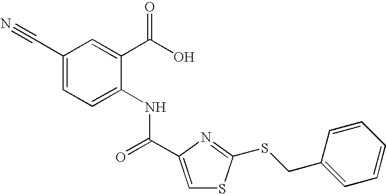 | 4 |
| 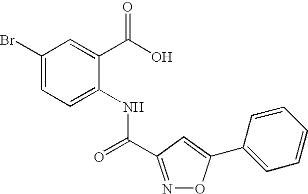 | 0.25 |
| 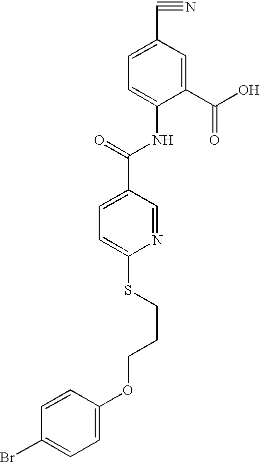 | 0.125 |
| 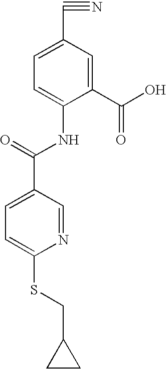 | 0.5 |
| 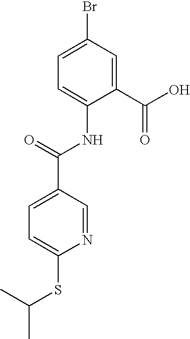 | 1 |
| 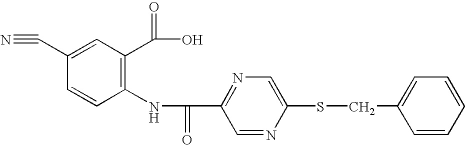 | 0.125 |
| 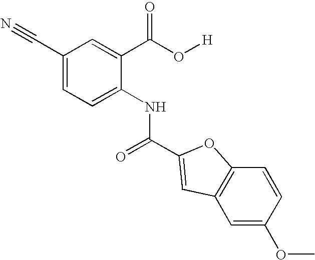 | 2 |
| 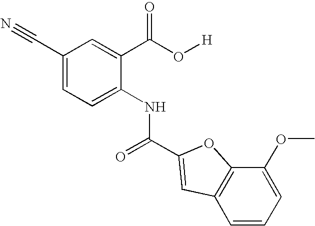 | 4 |
| 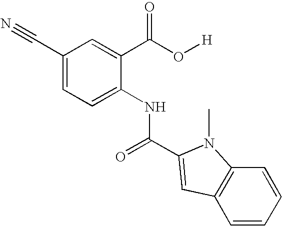 | 0.25 |
| 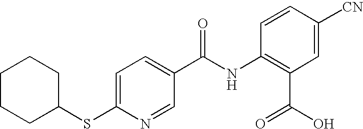 | 0.5 |
| 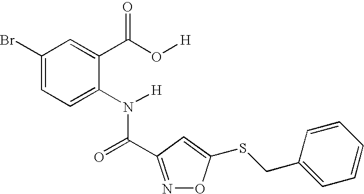 | 0.125 |
| 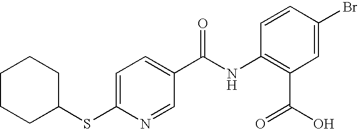 | 1 |
| 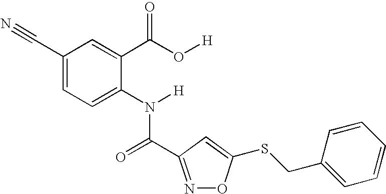 | 0.125 |
| 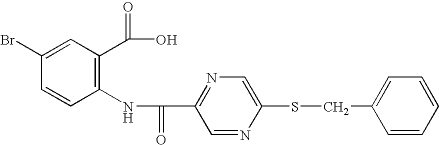 | 0.125 |
| 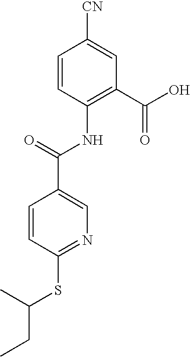 | 0.5 |
| 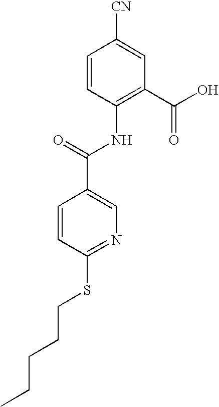 | 0.125 |
| 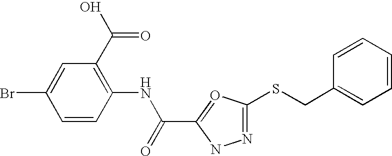 | 0.5 |
| 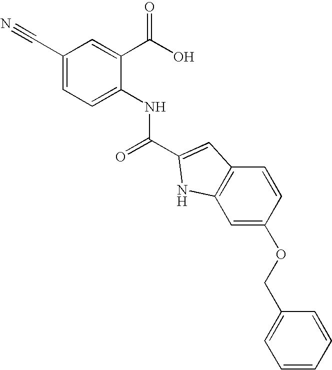 | 0.125 |
| 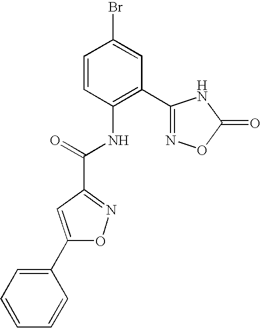 | 1 |
| 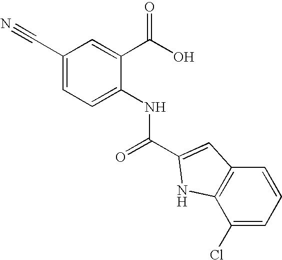 | 1 |
| 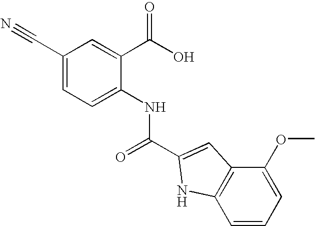 | 0.25 |
| 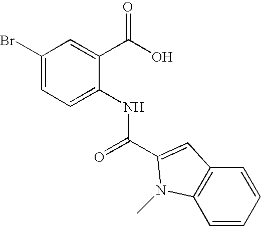 | 0.5 |
| 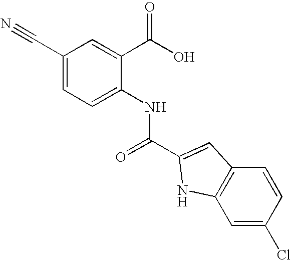 | 2 |
| 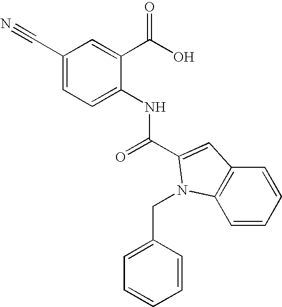 | 8 |
| 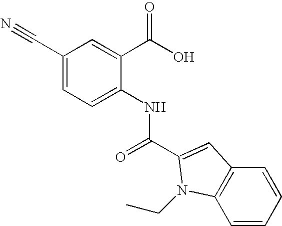 | 1 |
| 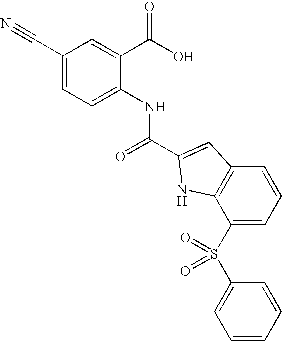 | 0.125 |
| 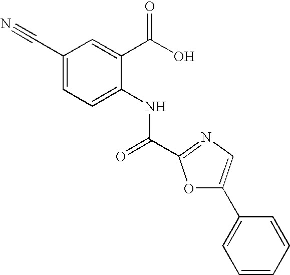 | 0.5 |
| 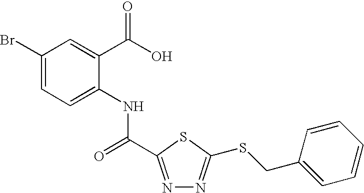 | 0.125 |
| 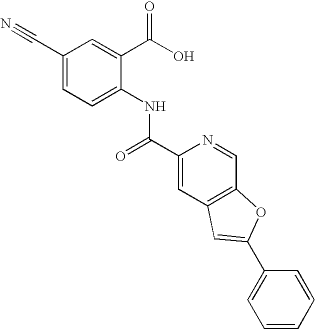 | 8 |
| 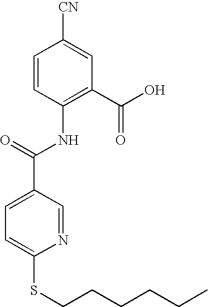 | 0.125 |
| 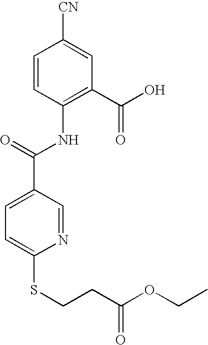 | 1 |
| 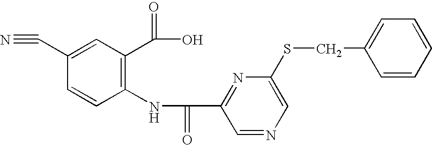 | 64 |
| 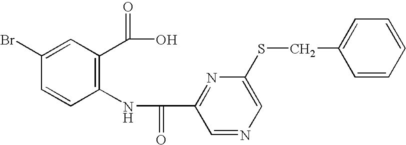 | 16 |
| 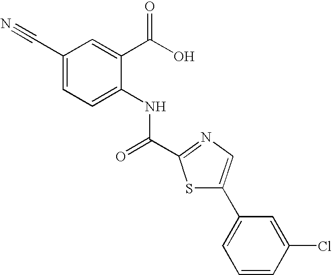 | 1 |
| 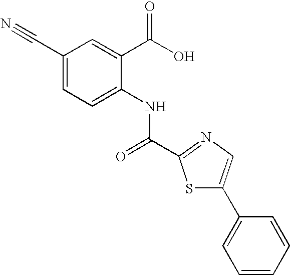 | 1 |
| 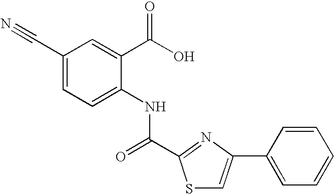 | 8 |
| 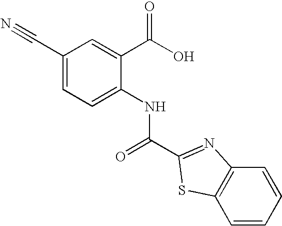 | 0.25 |
| 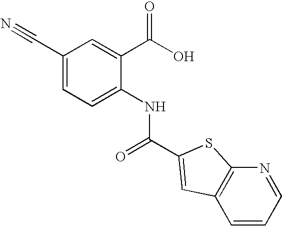 | 4 |
| 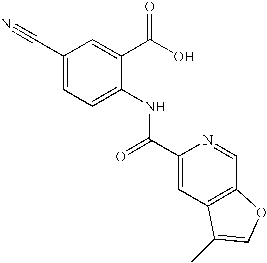 | 16 |
| 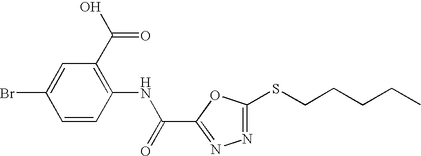 | 0.25 |
| 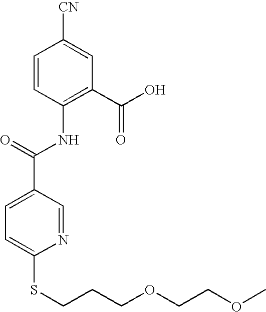 | 1 |
| 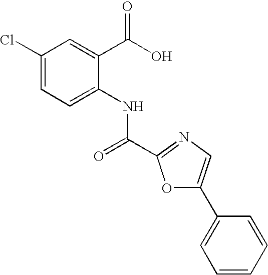 | 1 |
| 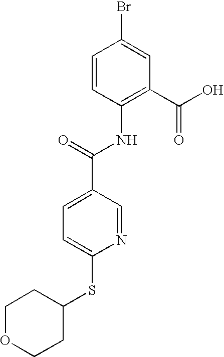 | 1 |
| 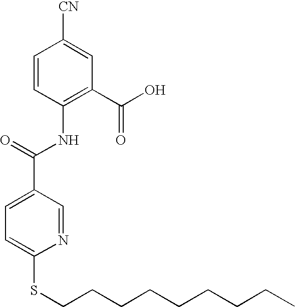 | 1 |
| 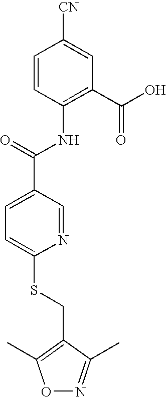 | 0.5 |
| 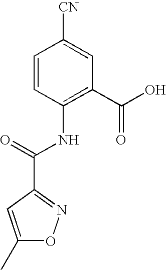 | 4 |
| 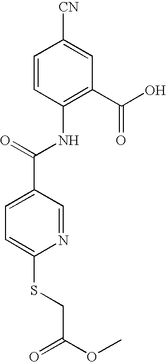 | 0.5 |
| 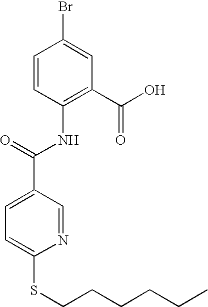 | 0.125 |
| 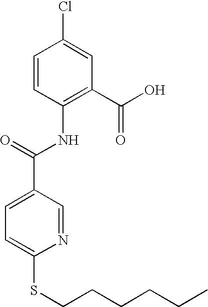 | 0.125 |
| 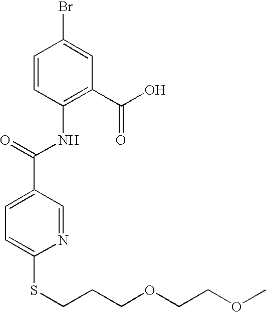 | 0.5 |
| 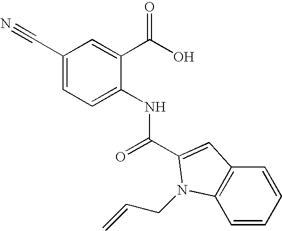 | 1 |
| 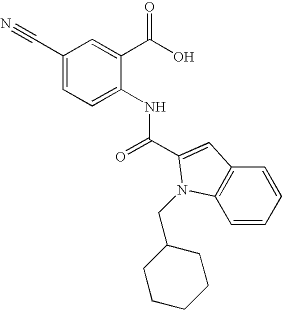 | 1 |
| 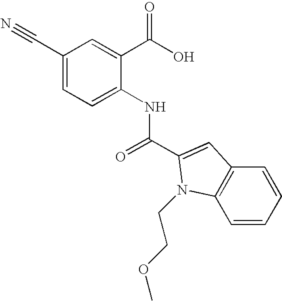 | 0.5 |
| 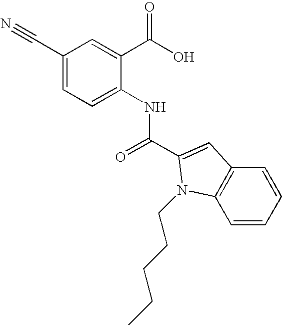 | 2 |
| 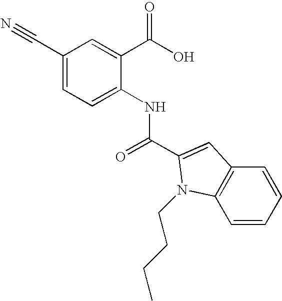 | 32 |
| 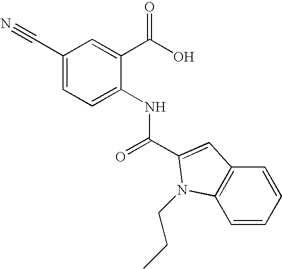 | 2 |
| 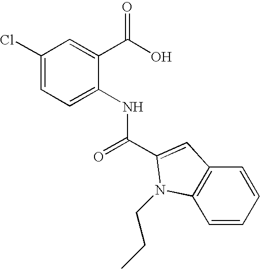 | 4 |
| 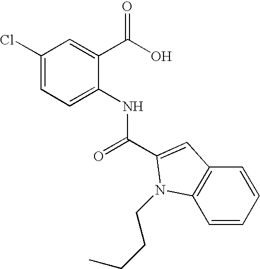 | 4 |
| 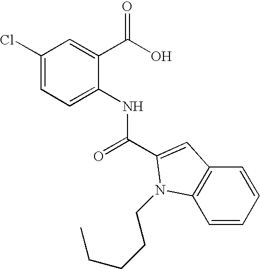 | 4 |
| 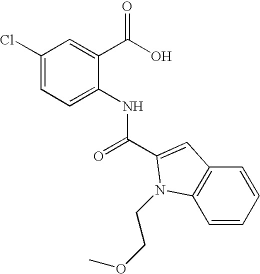 | 16 |
| 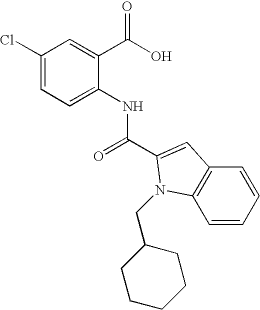 | 0.5 |
| 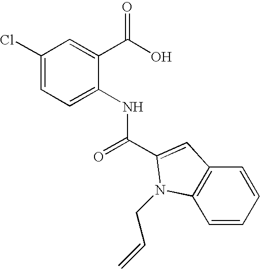 | 1 |
| 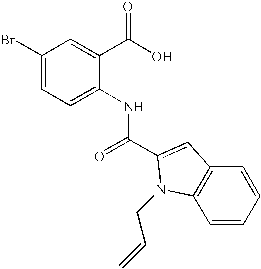 | 8 |
| 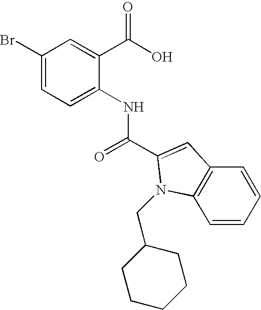 | 4 |
| 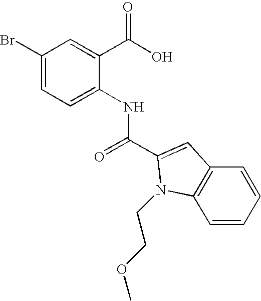 | 16 |
| 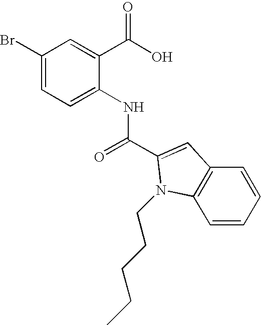 | 2 |
| 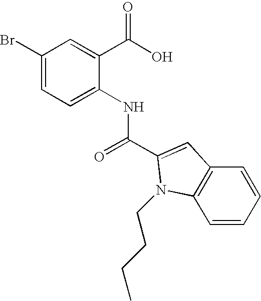 | 4 |
| 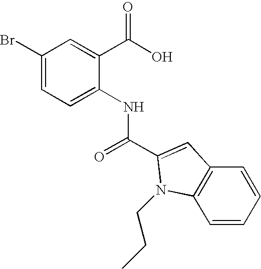 | 4 |
| 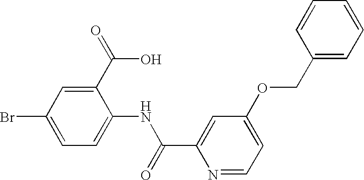 | 4 |
| 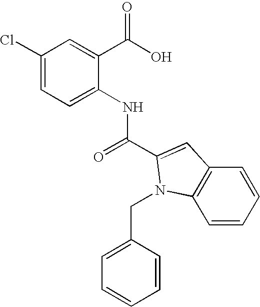 | 4 |
| 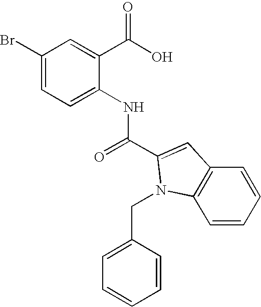 | 4 |
| 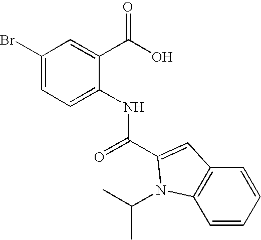 | 4 |
| 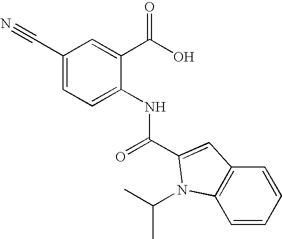 | 2 |
| 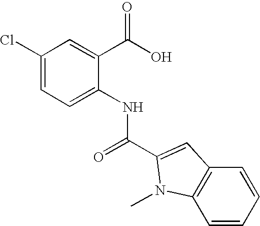 | 4 |
| 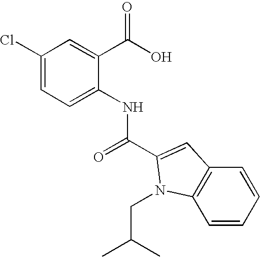 | 2 |
| 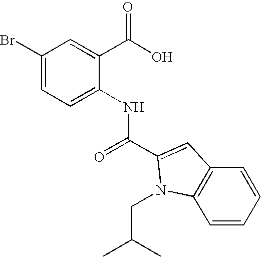 | 2 |
| 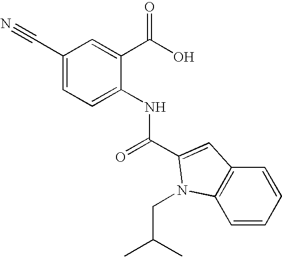 | 4 |
| 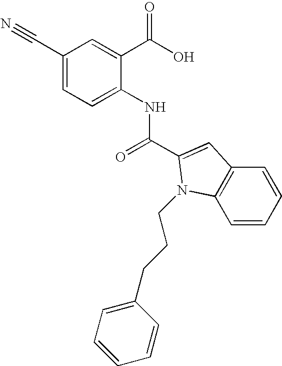 | 4 |
| 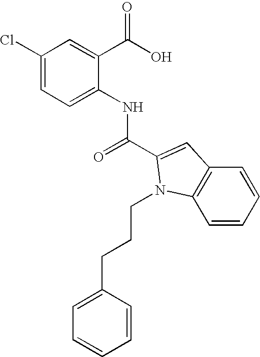 | 4 |
| 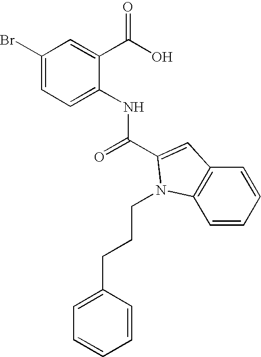 | 4 |
| 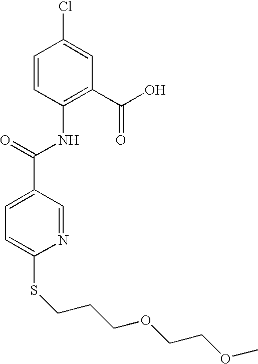 | 4 |
| 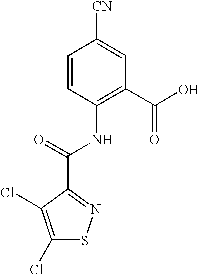 | 4 |
| 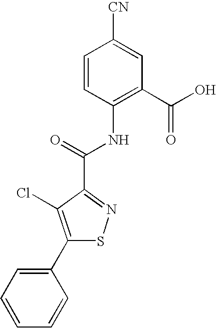 | 0.5 |
| 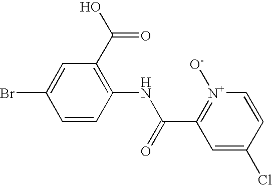 | >128 |
| 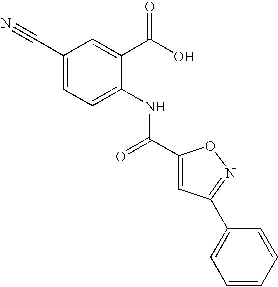 | 0.5 |
| 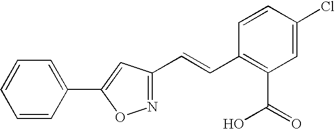 | 32 |
| 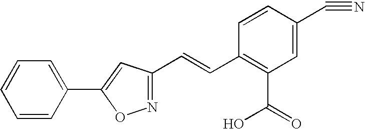 | 64 |
| 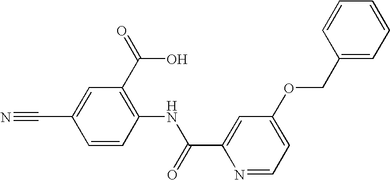 | 0.5 |
| 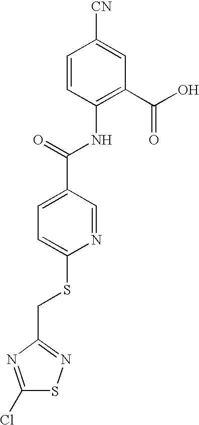 | 0.25 |
| 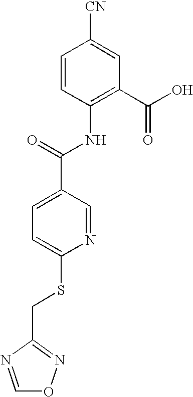 | 1 |
| 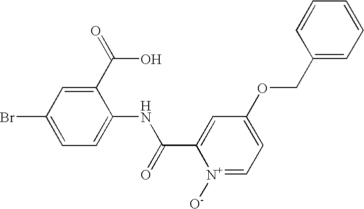 | 32 |
| 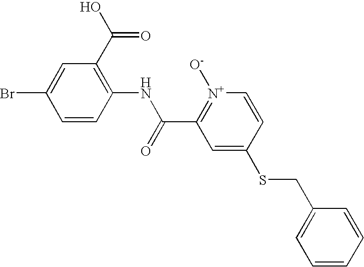 | 8 |
| 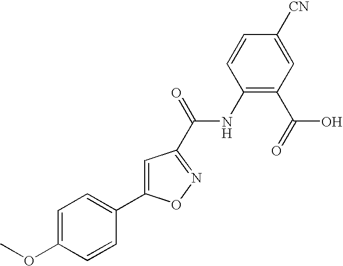 | 0.5 |
| 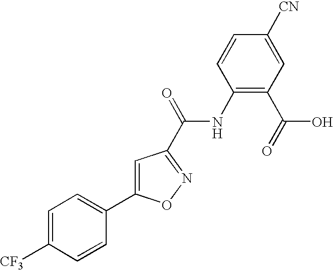 | 1 |
| 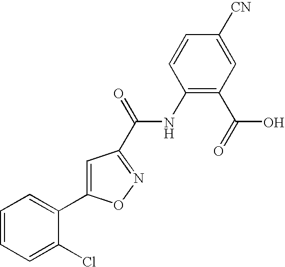 | 0.125 |
| 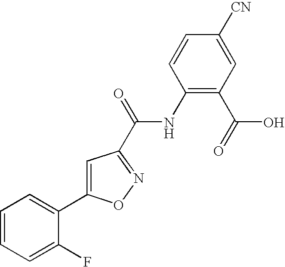 | 0.125 |
| 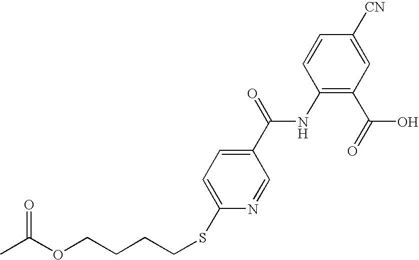 | 0.25 |
| 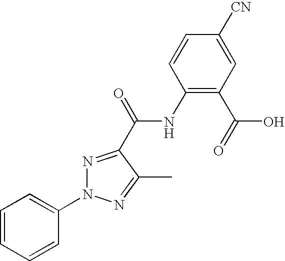 | 4 |
| 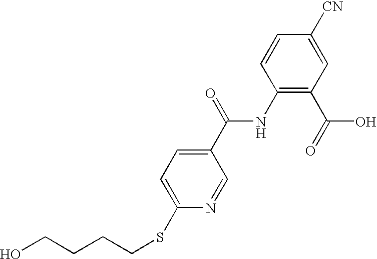 | 2 |
| 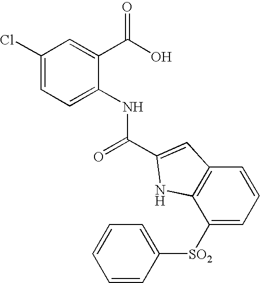 | 0.125 |
| 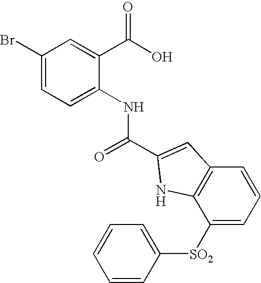 | 0.125 |
| 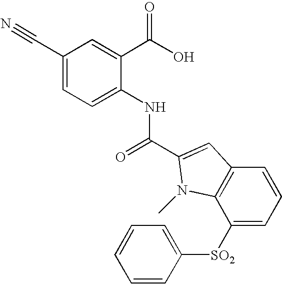 | 0.125 |
| 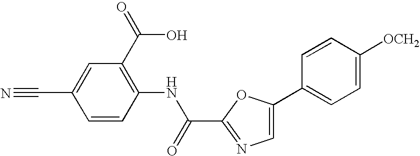 | 1 |
| 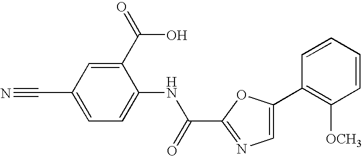 | 1 |
| 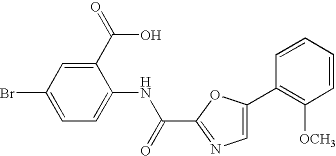 | 2 |
| 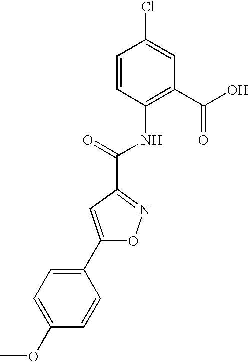 | 0.25 |
| 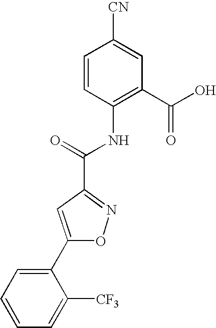 | 0.25 |
| 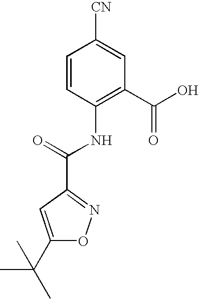 | 1 |
| 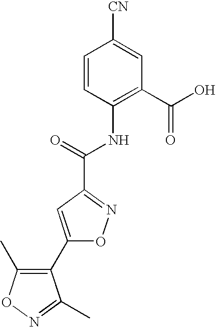 | 1 |
| 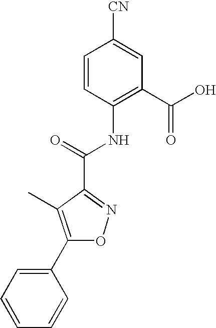 | 0.25 |
| 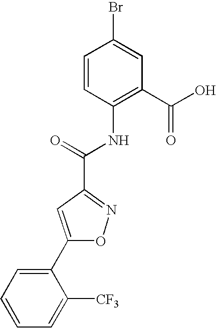 | 0.5 |
| 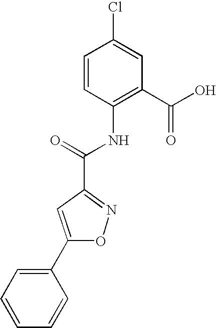 | 1 |
| 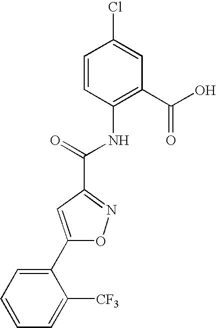 | 1 |
| 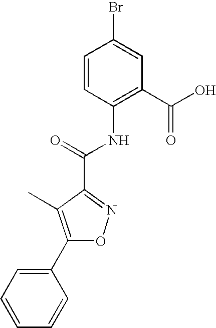 | 0.5 |
| 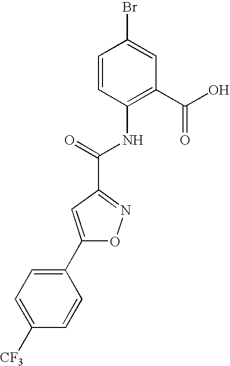 | 1 |
| 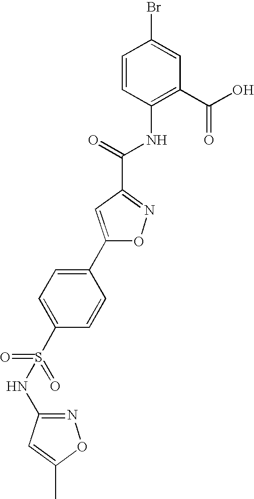 | 32 |
| 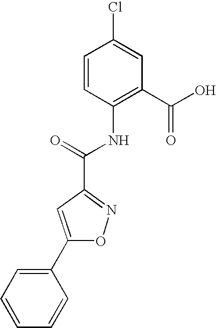 | 0.5 |
| 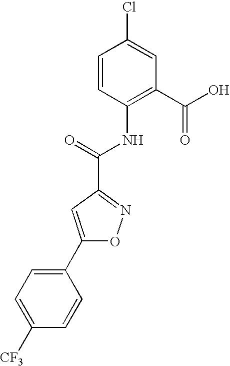 | 0.5 |
| 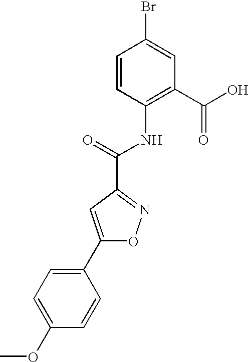 | 0.5 |
| 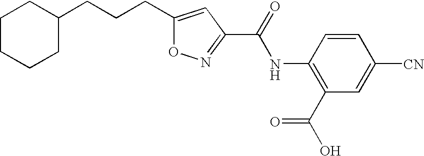 | 0.25 |
| 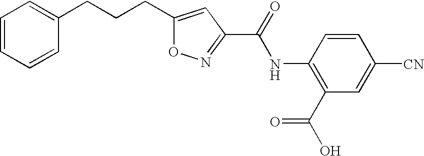 | 0.125 |
| 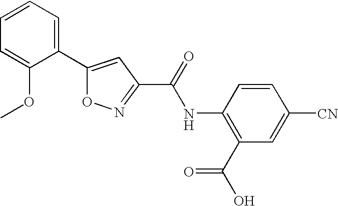 | 0.125 |
| 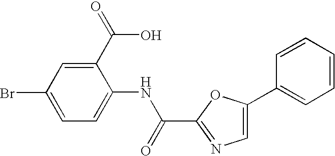 | 1 |
| 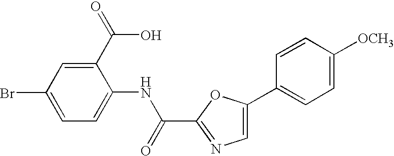 | 2 |
| 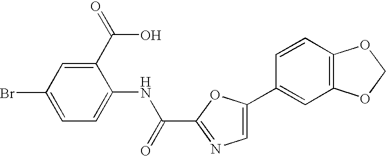 | 2 |
| 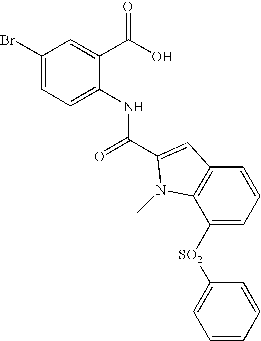 | 0.125 |
| 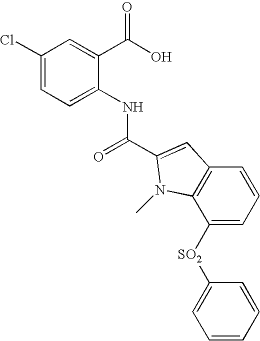 | 0.125 |
| 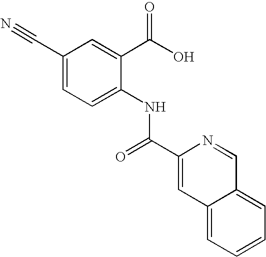 | 1 |
| 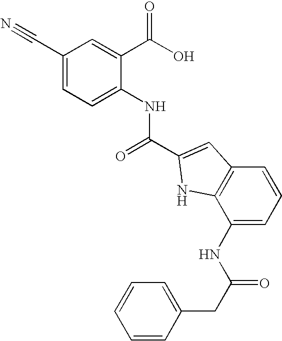 | 0.125 |
| 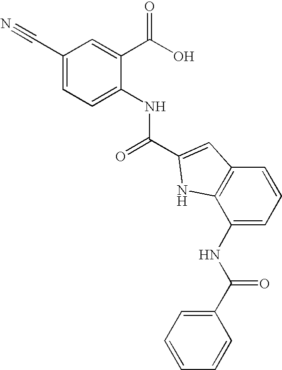 | 2 |
| 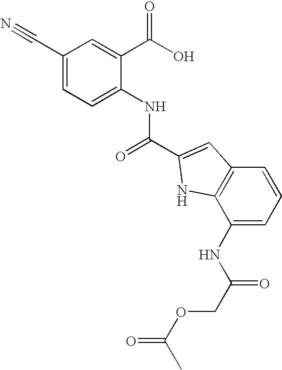 | 2 |
| 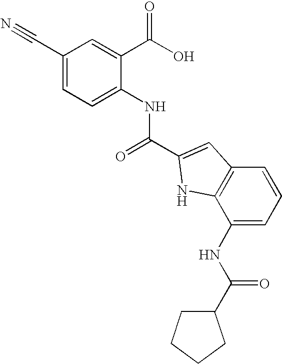 | 2 |
| 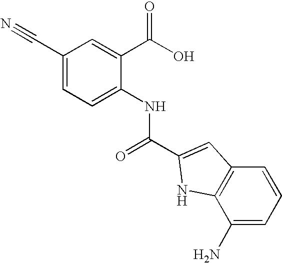 | 1 |
| 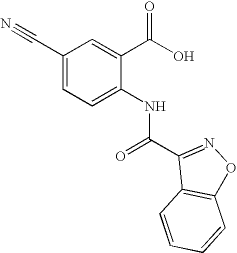 | 0.125 |
| 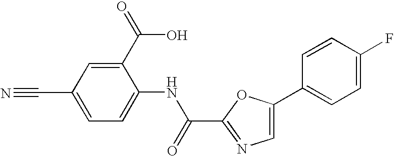 | 1 |
| 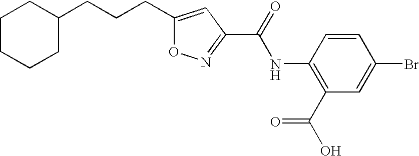 | 4 |
| 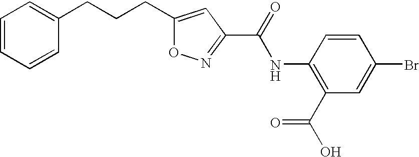 | 0.25 |
| 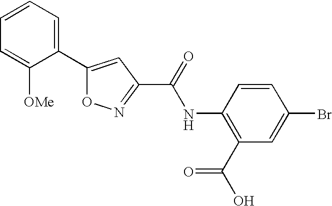 | 0.125 |
| 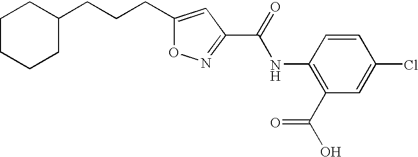 | 4 |
| 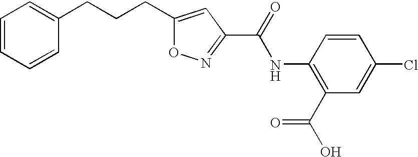 | 0.5 |
| 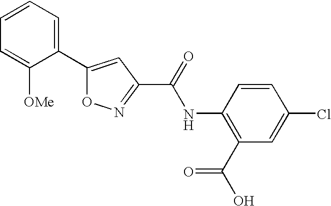 | 0.125 |
| 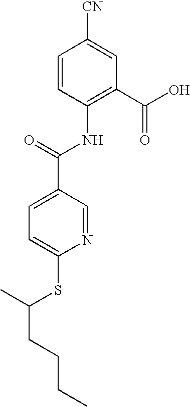 | 0.125 |
| 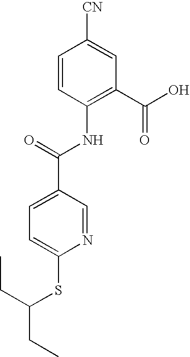 | 0.25 |
| 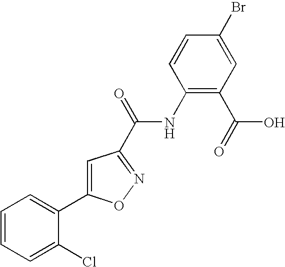 | 0.125 |
| 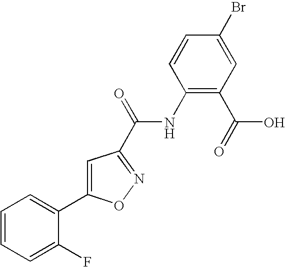 | 0.5 |
| 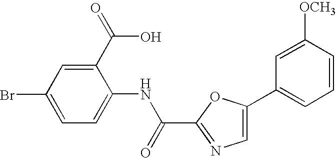 | 1 |
| 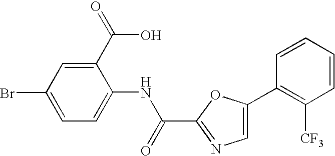 | 1 |
| 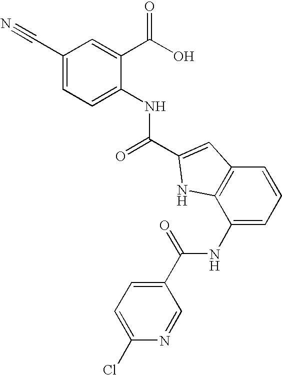 | 1 |
| 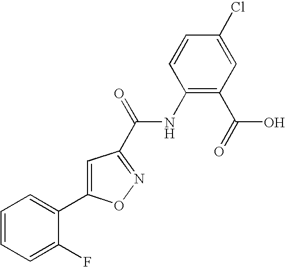 | 0.25 |
| 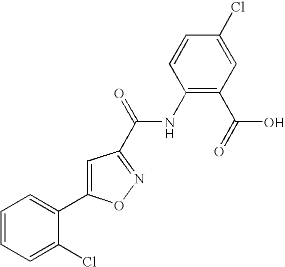 | 0.5 |
| 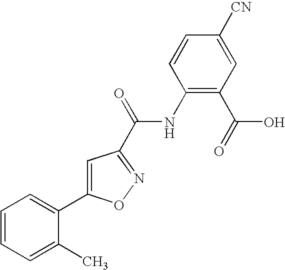 | 0.25 |
| 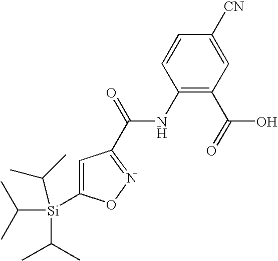 | 0.5 |
| 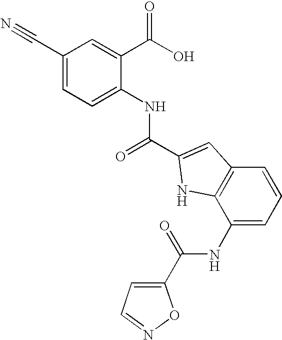 | 2 |
| 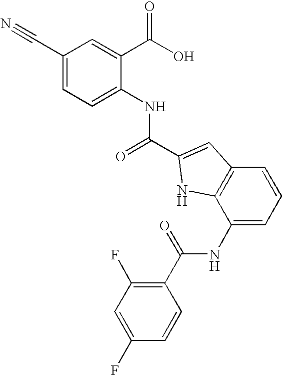 | 1 |
| 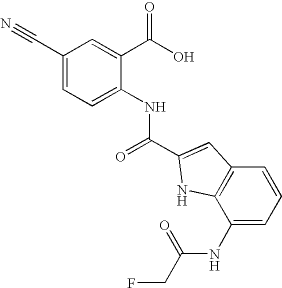 | 2 |
| 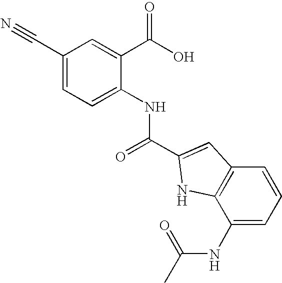 | 2 |
| 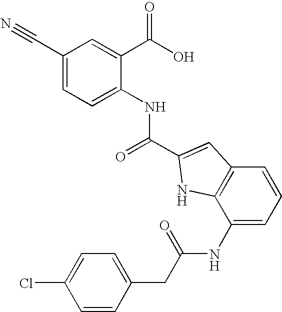 | 0.25 |
| 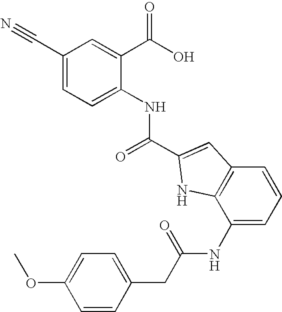 | 0.25 |
| 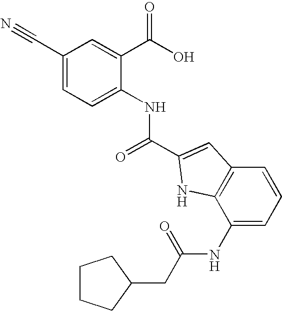 | 1 |
| 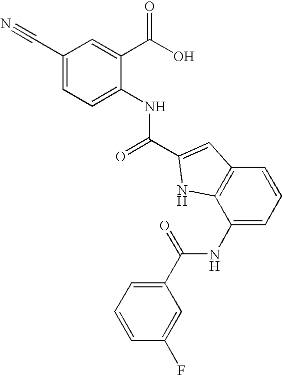 | 0.5 |
| 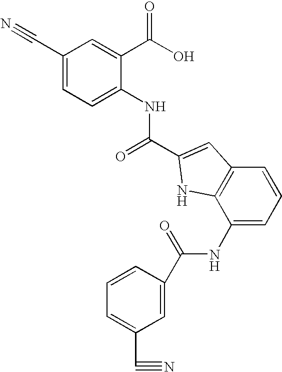 | 1 |
| 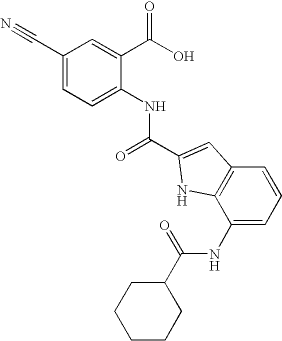 | 2 |
| 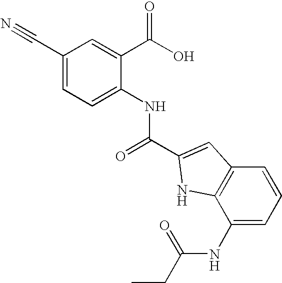 | 2 |
| 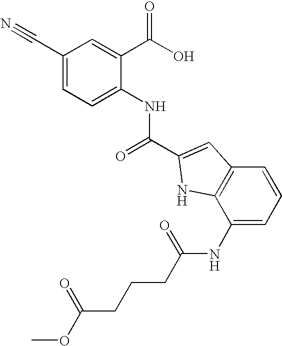 | 0.5 |
| 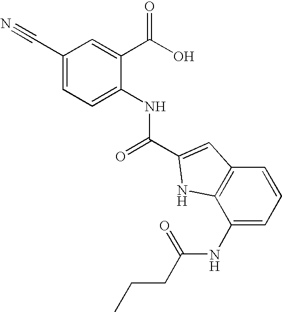 | 2 |
| 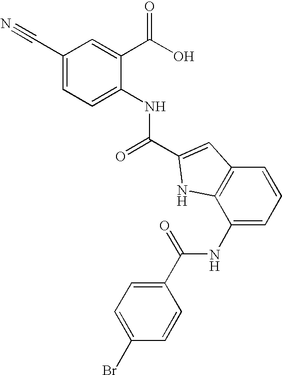 | 0.25 |
| 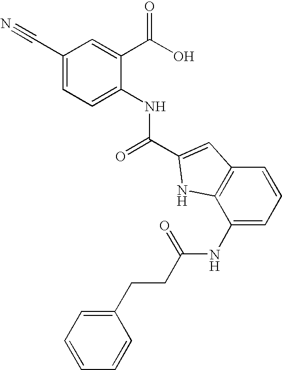 | 0.5 |
| 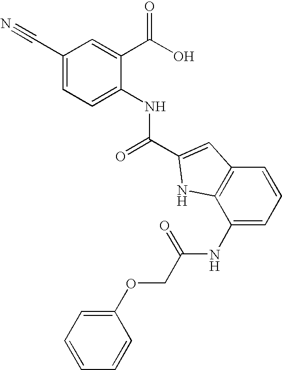 | 0.5 |
| 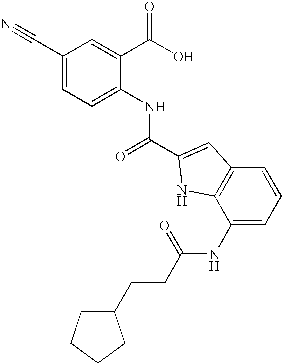 | 0.25 |
| 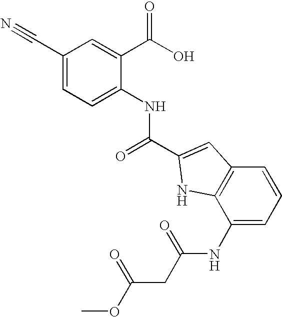 | 4 |
| 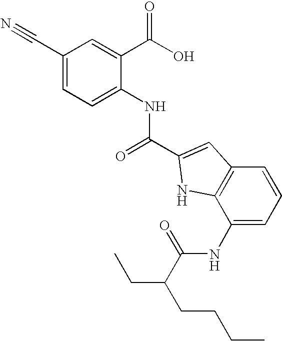 | 0.5 |
| 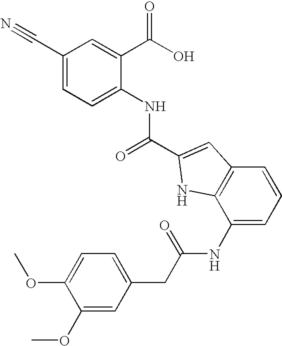 | 1 |
| 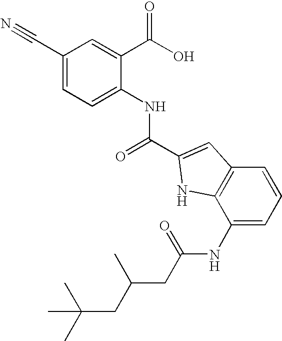 | 0.5 |
| 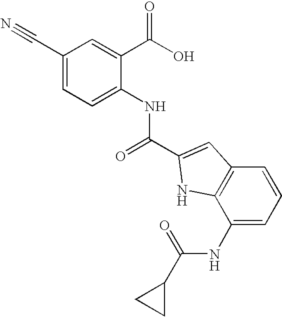 | 1 |
| 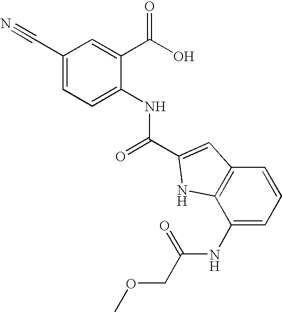 | 4 |
| 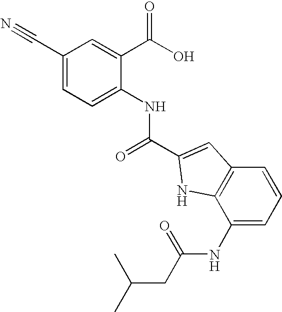 | 2 |
| 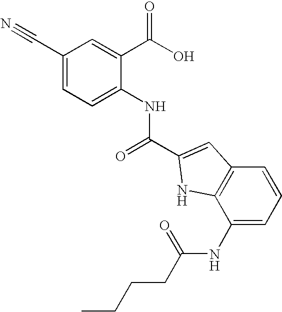 | 0.5 |
| 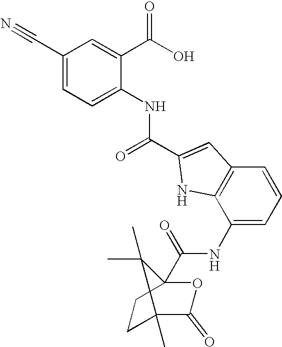 | 8 |
| 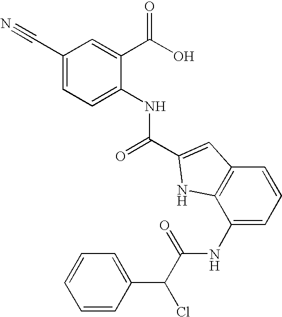 | 0.125 |
| 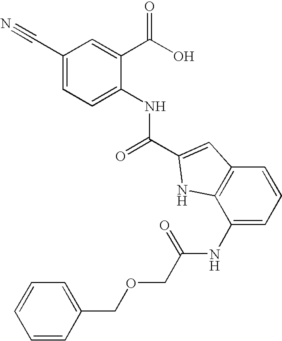 | 1 |
| 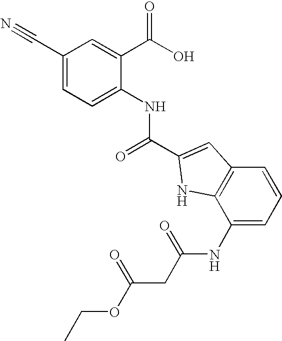 | 0.5 |
| 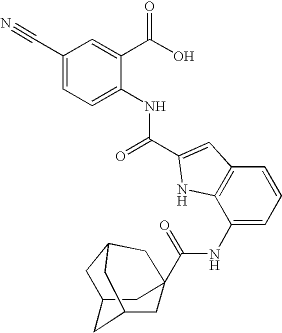 | 16 |
| 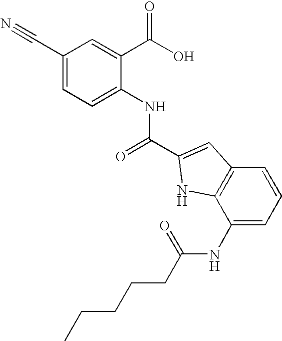 | 0.5 |
| 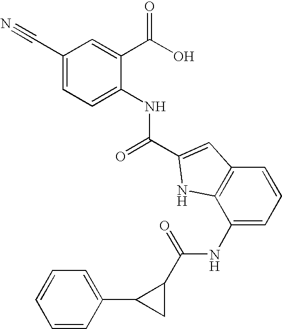 | 0.5 |
| 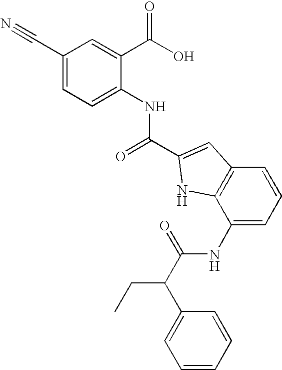 | 0.5 |
| 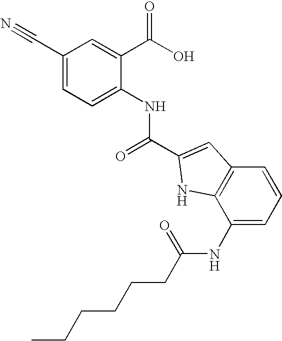 | 0.5 |
| 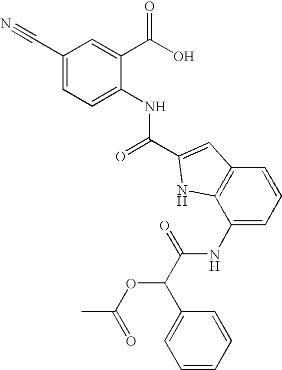 | 1 |
| 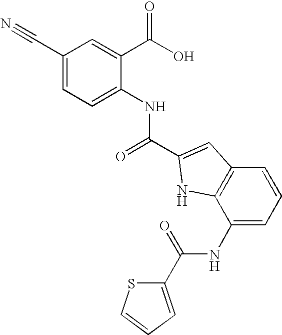 | 0.25 |
| 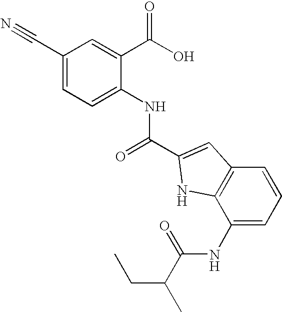 | 1 |
| 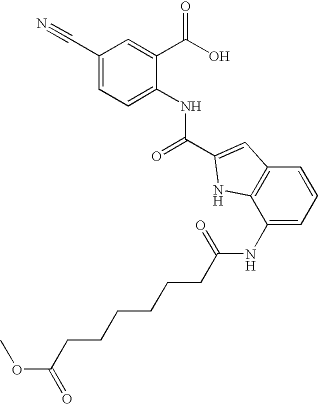 | 1 |
| 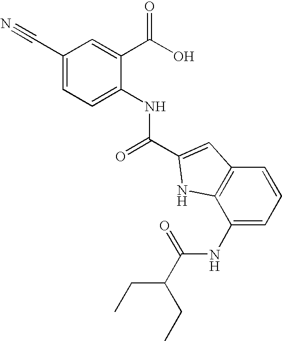 | 1 |
| 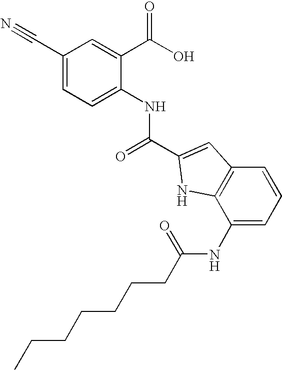 | 0.125 |
| 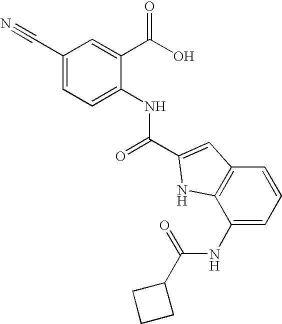 | 0.5 |
| 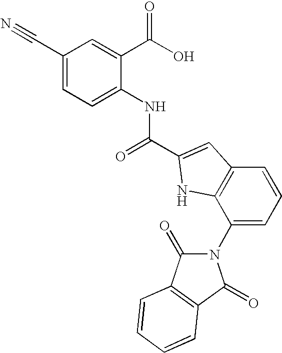 | 8 |
| 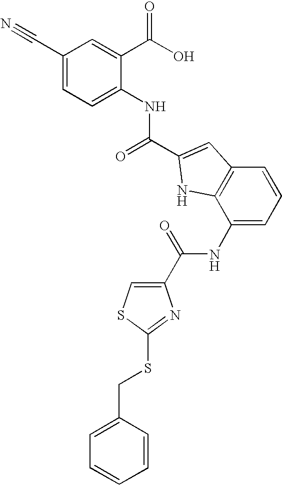 | >128 |
| 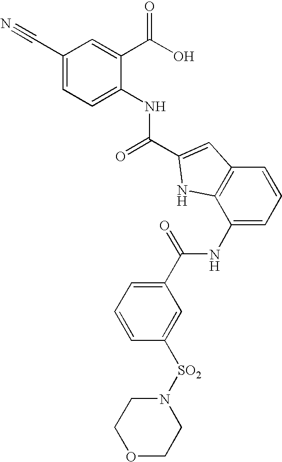 | 0.5 |
| 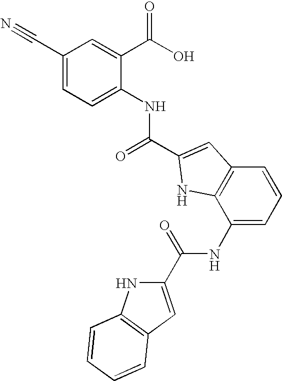 | 0.25 |
| 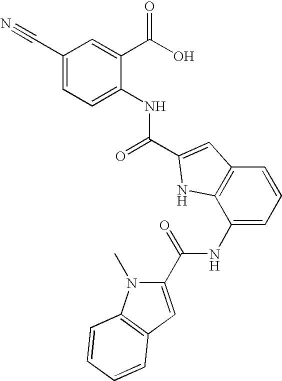 | 0.125 |
| 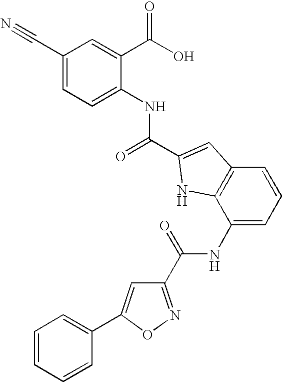 | 1 |
| 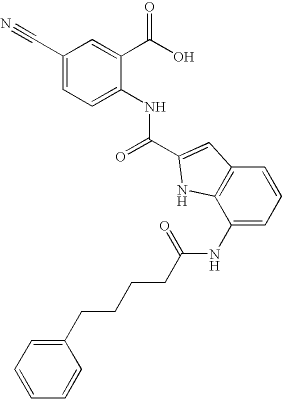 | 0.25 |
| 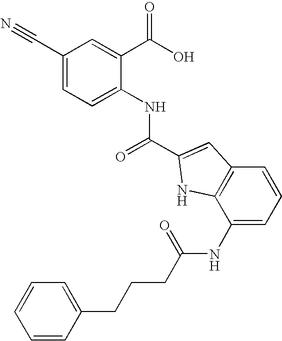 | 0.5 |
| 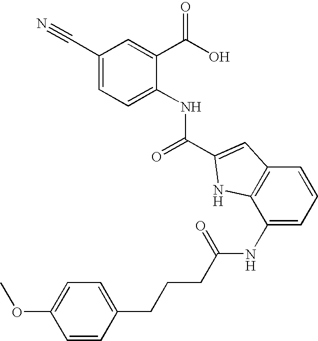 | 1 |
| 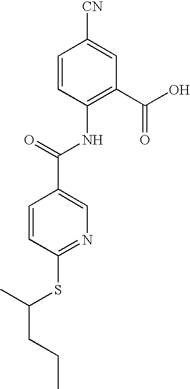 | 0.125 |
| 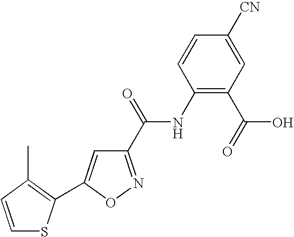 | 0.125 |
| 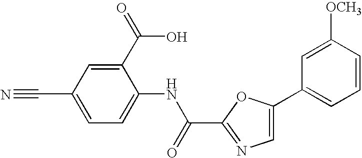 | 0.5 |
| 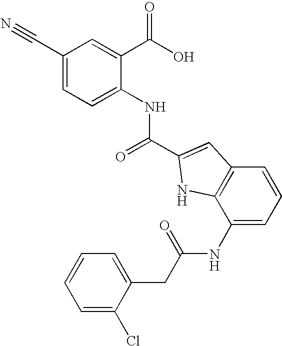 | 0.25 |
| 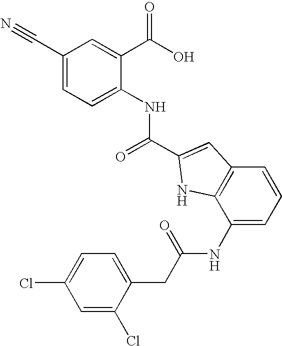 | 0.25 |
| 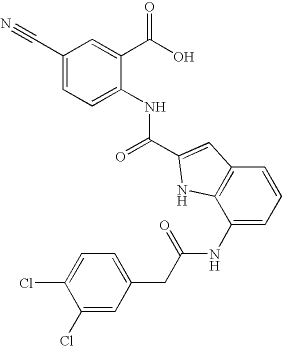 | 0.125 |
| 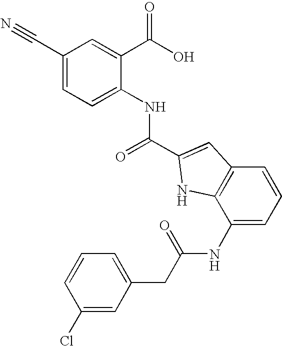 | 0.25 |
| 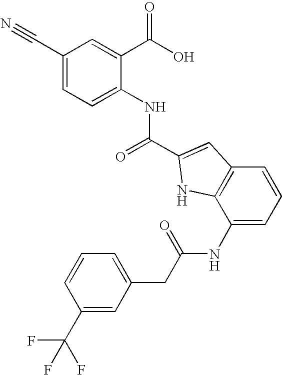 | 0.25 |
| 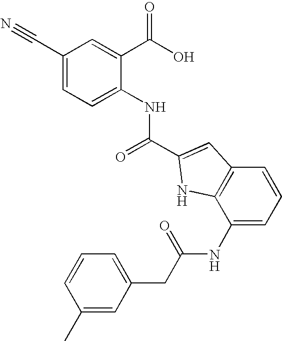 | 0.125 |
| 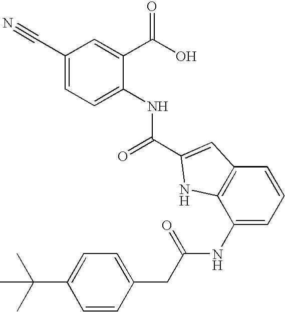 | 0.5 |
| 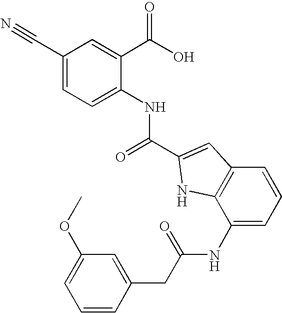 | 0.5 |
| 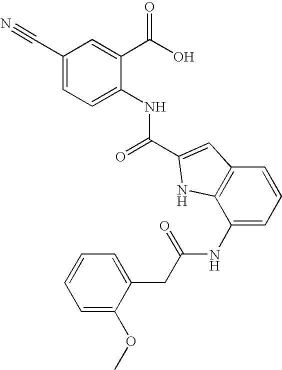 | 0.5 |
| 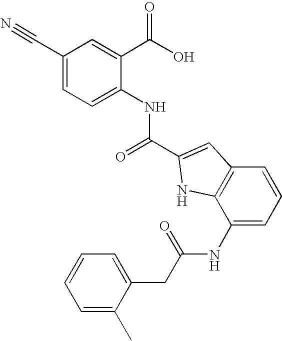 | 0.25 |
| 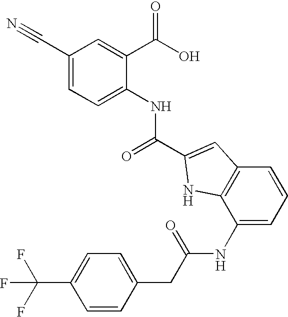 | 0.5 |
| 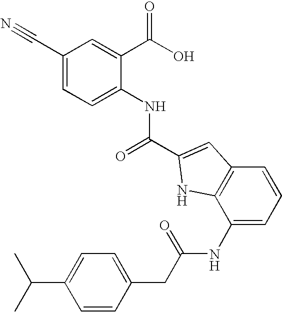 | 0.5 |
| 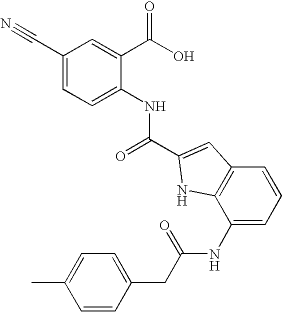 | 0.5 |
| 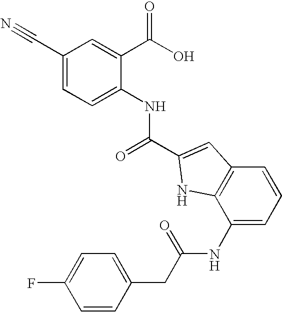 | 0.25 |
| 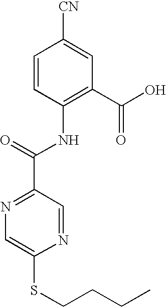 | 0.125 |
| 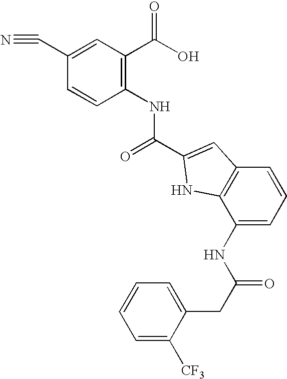 | 1 |
| 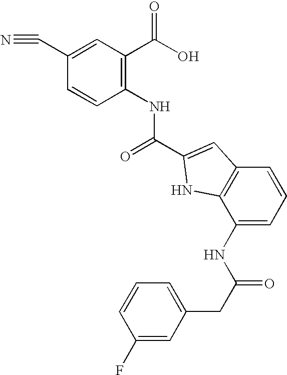 | 0.25 |
| 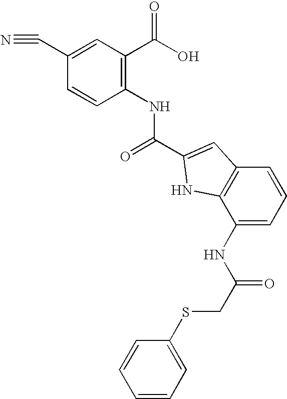 | 0.5 |
| 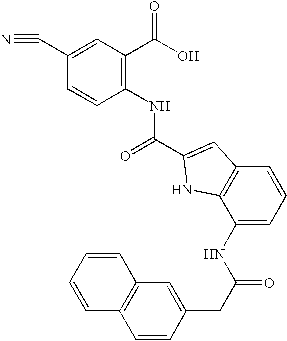 | 0.25 |
| 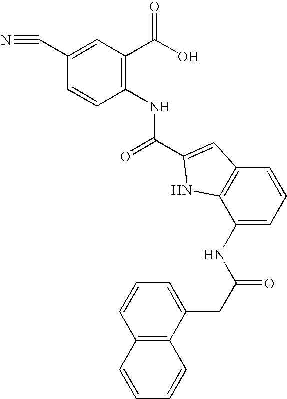 | 0.125 |
| 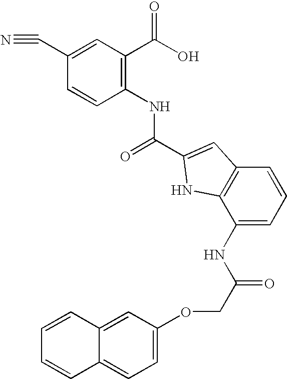 | 0.25 |
| 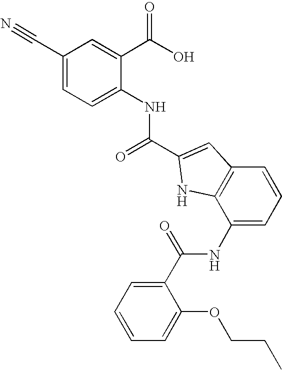 | 1 |
| 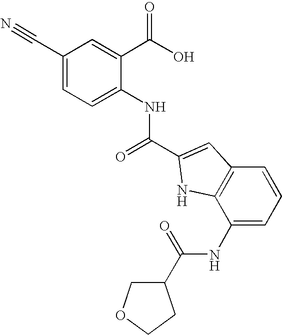 | 2 |
| 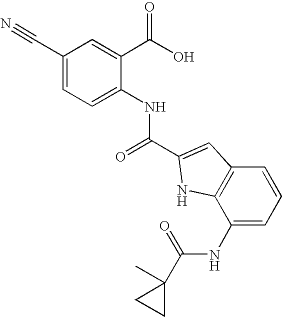 | 2 |
| 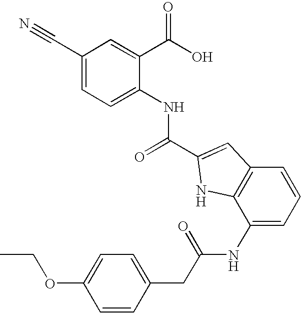 | 0.5 |
| 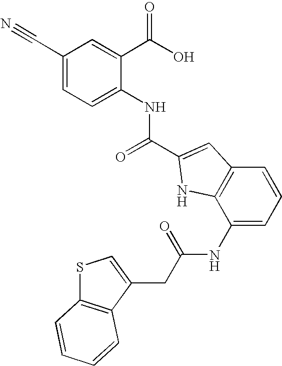 | 0.25 |
| 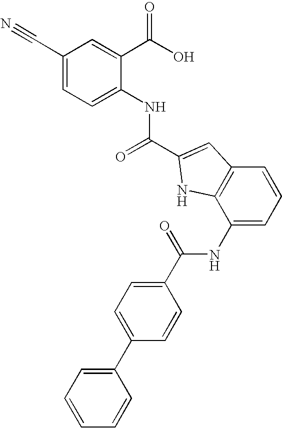 | 0.5 |
| 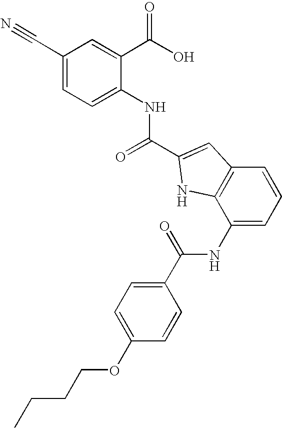 | 2 |
| 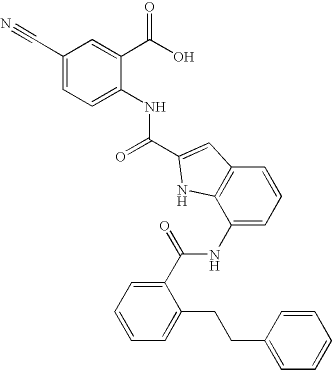 | 0.5 |
| 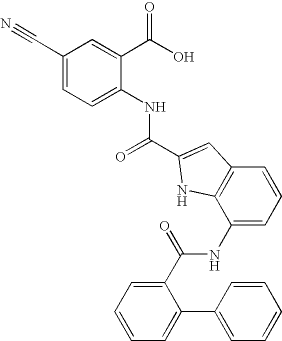 | 1 |
| 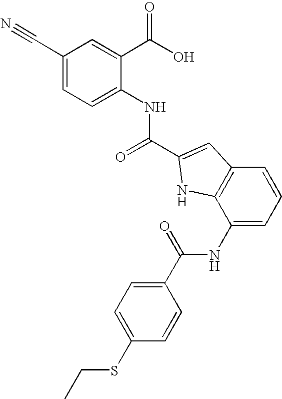 | 0.25 |
| 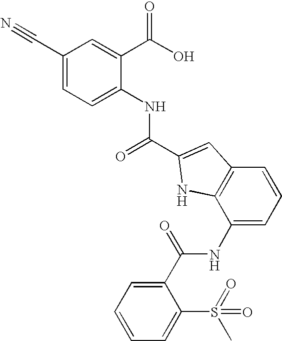 | 4 |
| 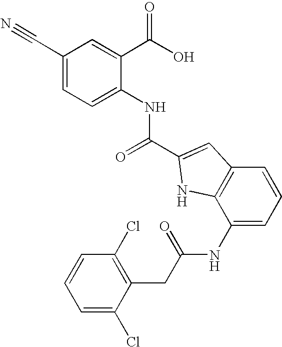 | 0.25 |
| 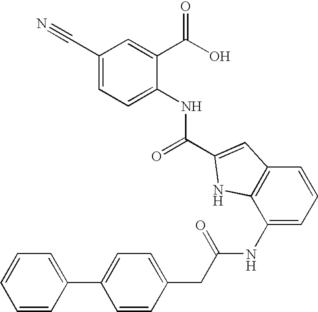 | 0.5 |
| 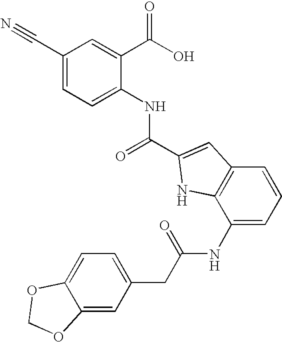 | 0.5 |
| 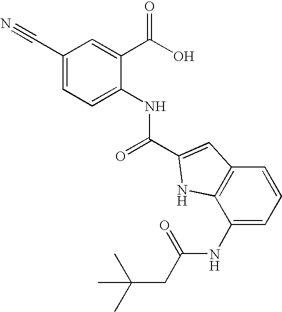 | 2 |
| 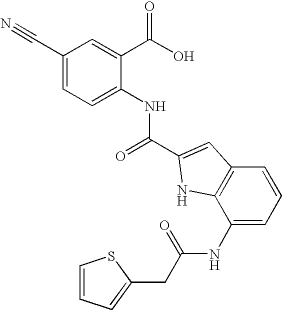 | 0.5 |
| 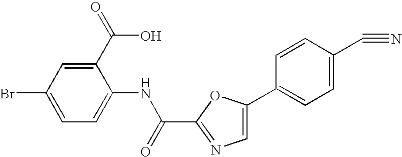 | 8 |
| 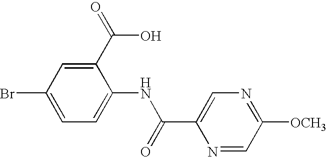 | 4 |
| 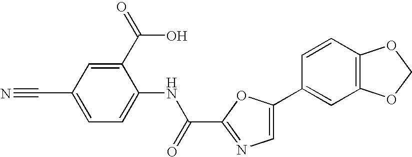 | 2 |
| 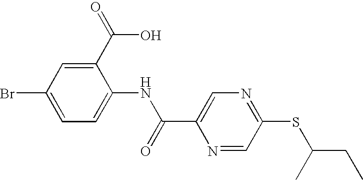 | 0.5 |
| 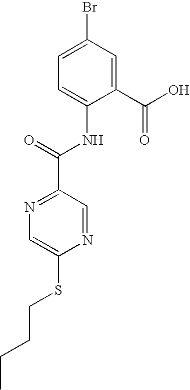 | 1 |
| 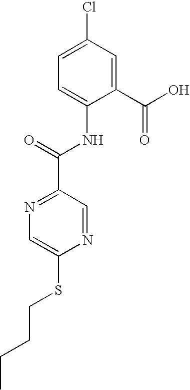 | 0.5 |
| 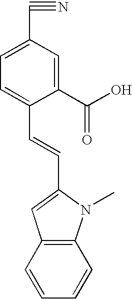 | 64 |
| 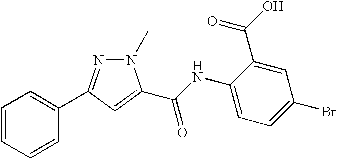 | 64 |
| 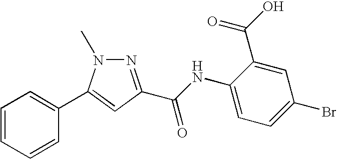 | 32 |
| 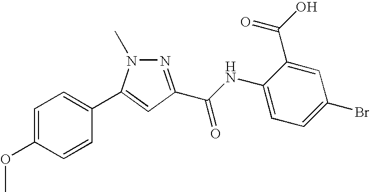 | >128 |
| 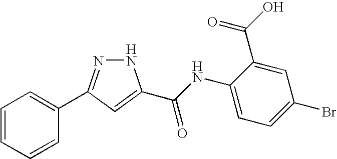 | >128 |
| 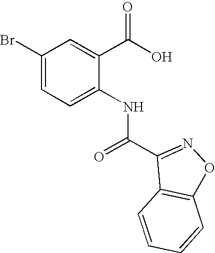 | 0.25 |
| 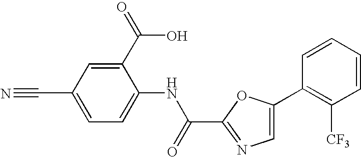 | 1 |
| 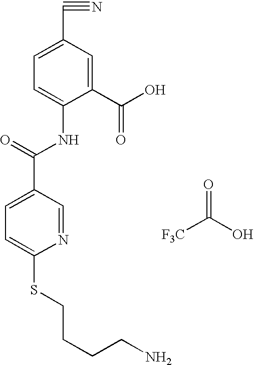 | 2 |
| 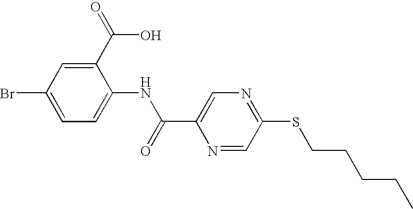 | 0.25 |
| 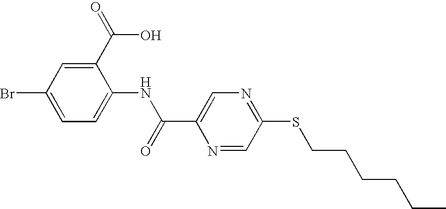 | 0.5 |
| 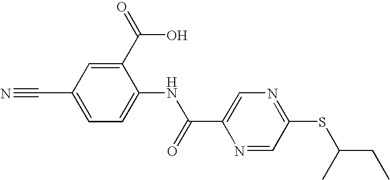 | 0.25 |
| 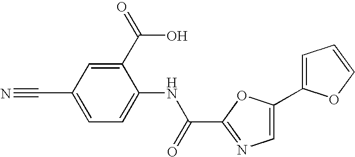 | 2 |
| 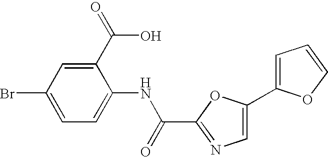 | 2 |
| 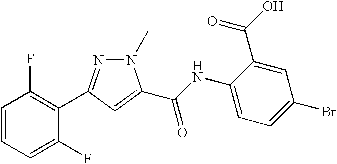 | 128 |
| 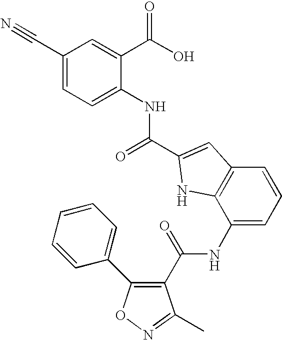 | 0.5 |
| 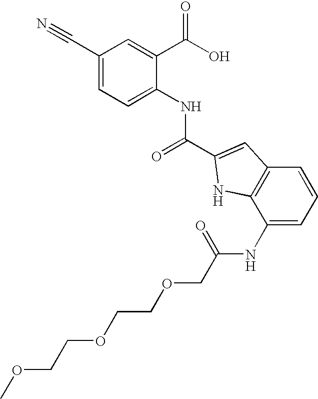 | 4 |
| 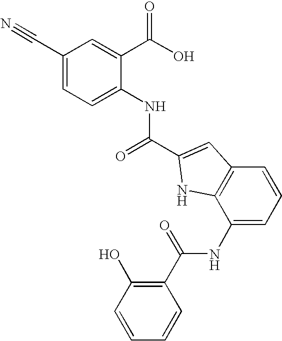 | 1 |
| 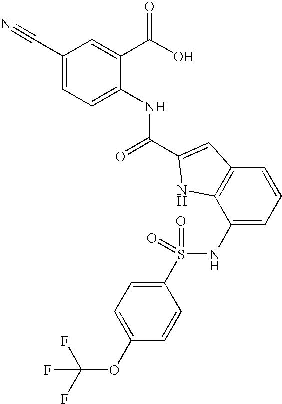 | 1 |
| 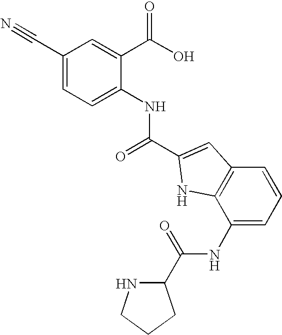 | >128 |
| 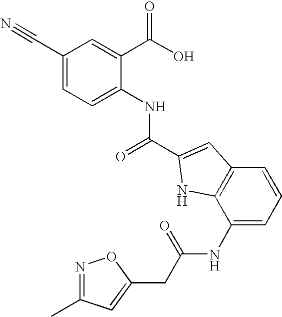 | 2 |
| 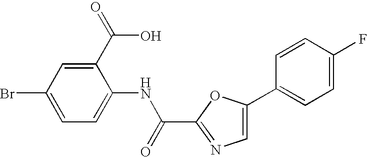 | 2 |
| 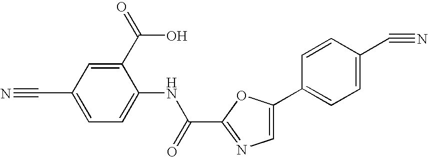 | >128 |
| 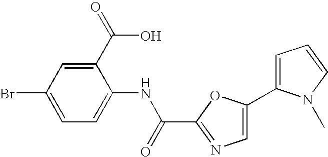 | 2 |
| 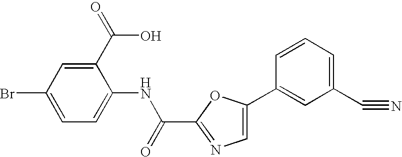 | 4 |
| 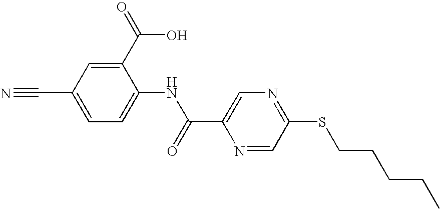 | 0.125 |
| 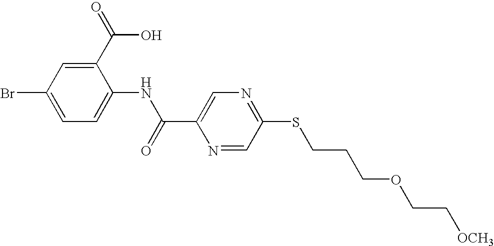 | 2 |
| 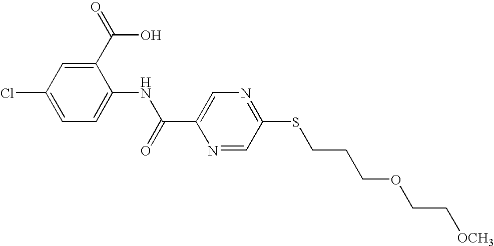 | 2 |
| 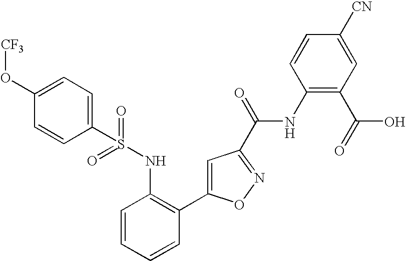 | 8 |
| 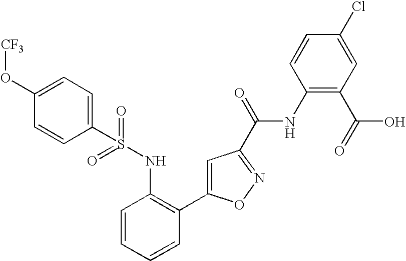 | 8 |
| 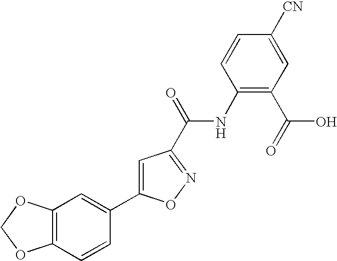 | 0.125 |
| 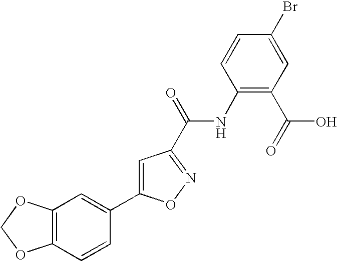 | 0.125 |
| 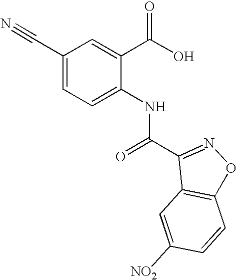 | 0.25 |
| 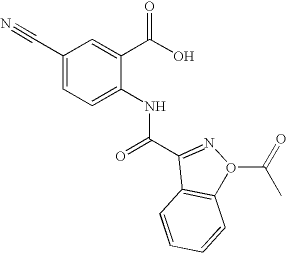 | 16 |
| 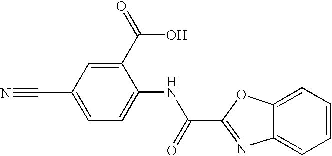 | 1 |
| 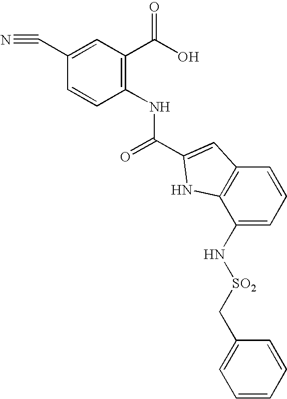 | 0.125 |
| 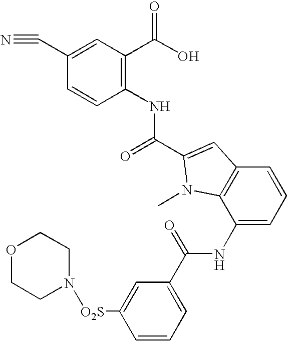 | 2 |
| 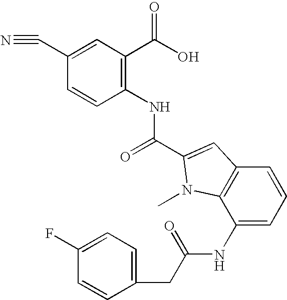 | 0.125 |
| 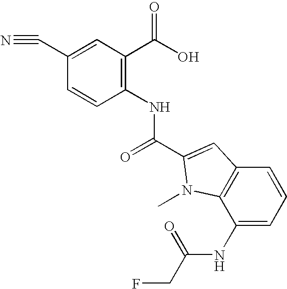 | 1 |
| 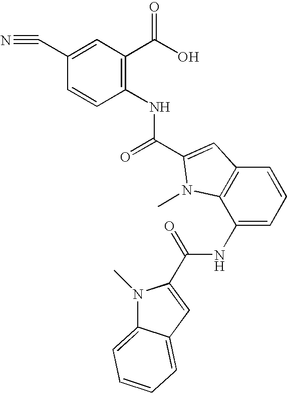 | 0.125 |
| 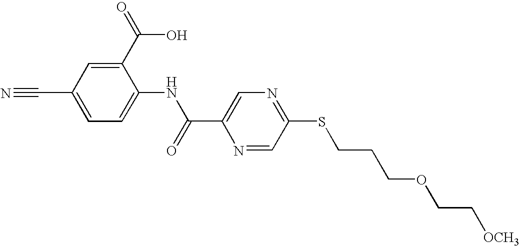 | 1 |
| 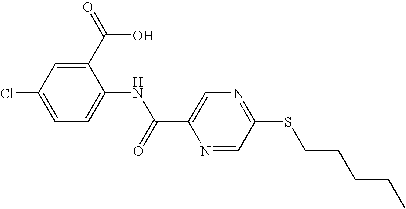 | 0.125 |
| 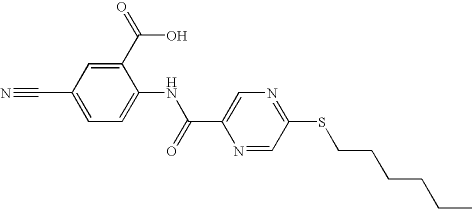 | 0.125 |
| 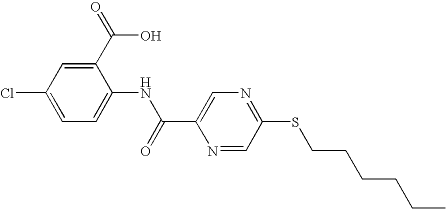 | 0.5 |
| 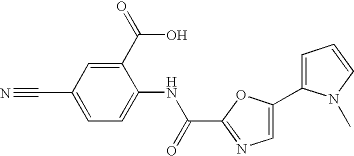 | 2 |
| 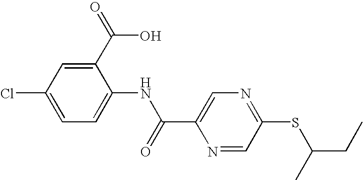 | 0.25 |
| 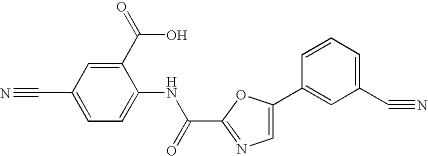 | 2 |
| 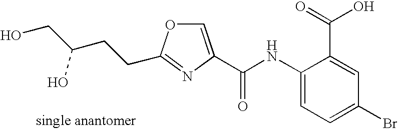 | >128 |
| 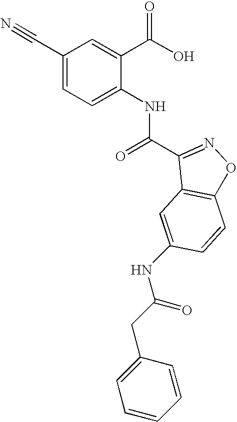 | 1 |
| 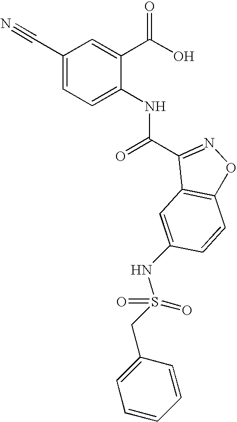 | 0.125 |
| 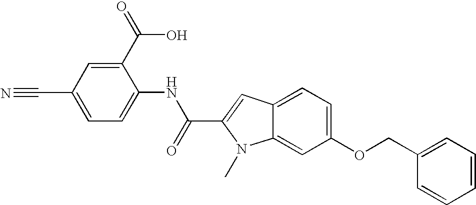 | 0.125 |
| 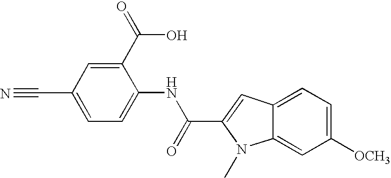 | 0.5 |
| 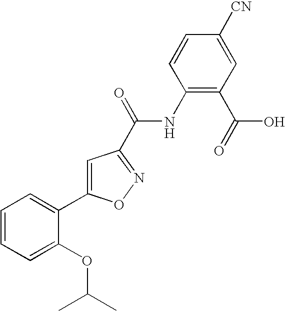 | 0.125 |
| 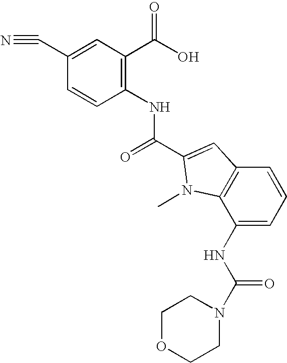 | 8 |
| 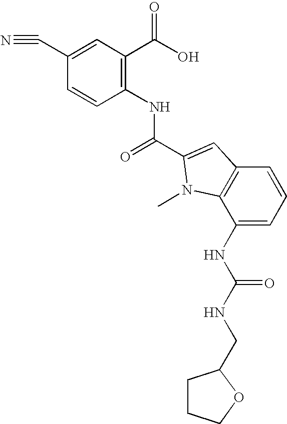 | 1 |
| 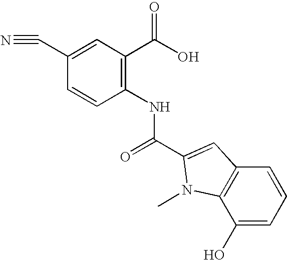 | 1 |
| 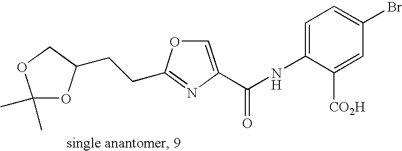 | >128 |
| 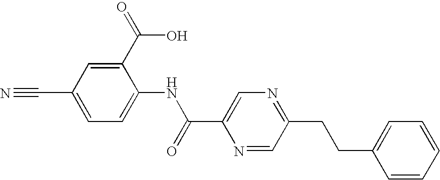 | 16 |
| 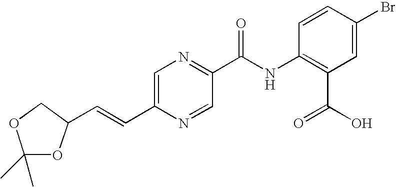 | 32 |
| 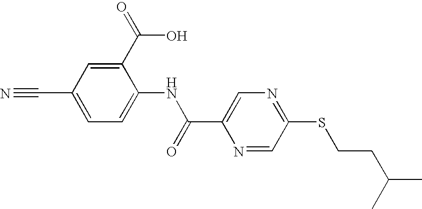 | 0.125 |
| 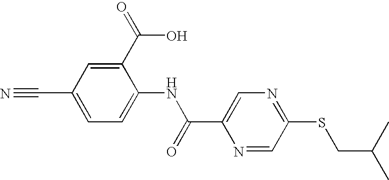 | 0.25 |
| 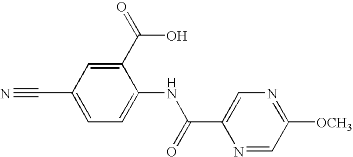 | 8 |
| 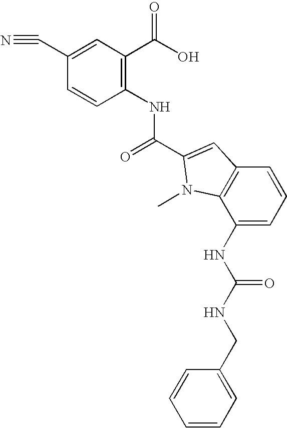 | 0.125 |
| 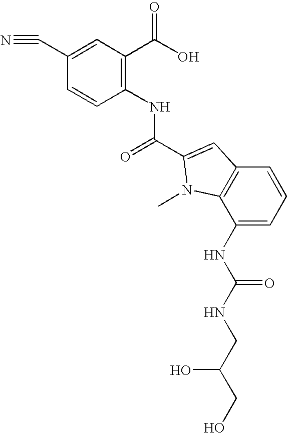 | 4 |
| 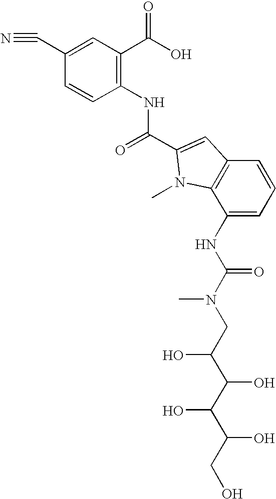 | 32 |
| 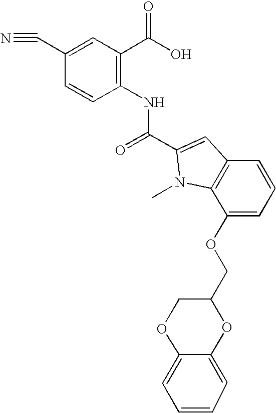 | 0.125 |
| 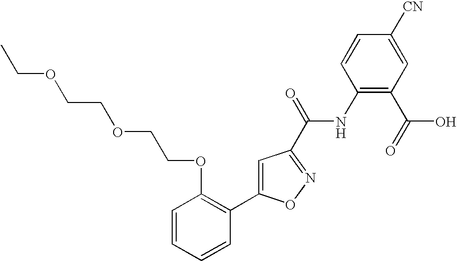 | 0.5 |
| 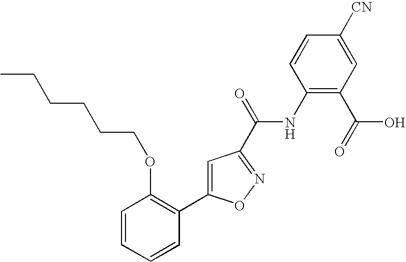 | 0.5 |
| 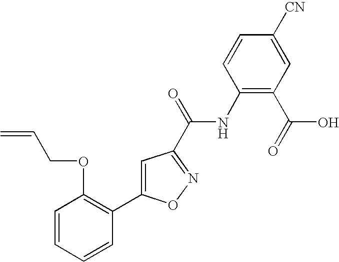 | 0.125 |
| 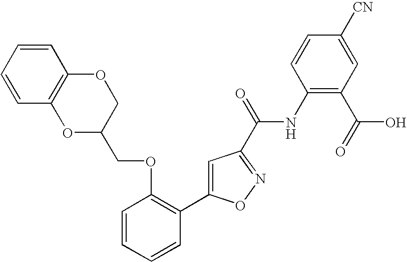 | 0.25 |
| 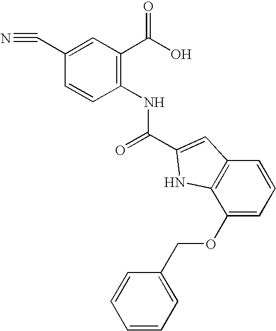 | 0.125 |
| 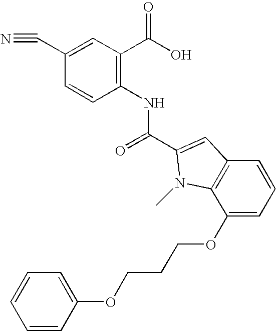 | 0.125 |
| 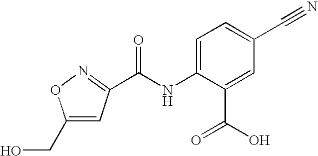 | 8 |
| 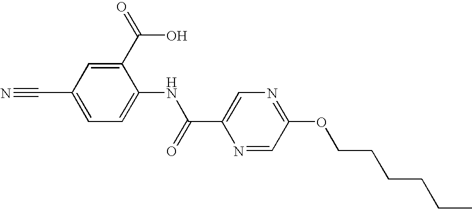 | 0.125 |
| 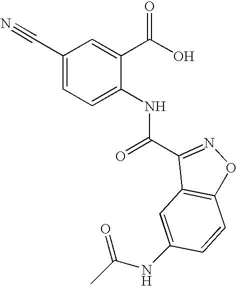 | 2 |
| 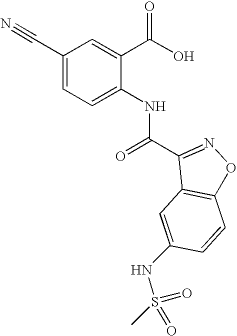 | 0.5 |
| 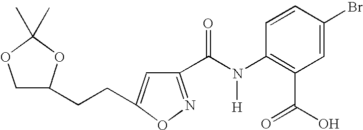 | 4 |
| 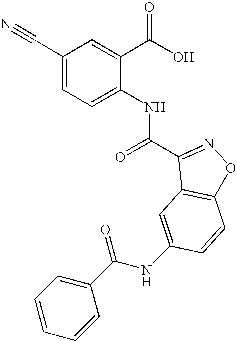 | 1 |
| 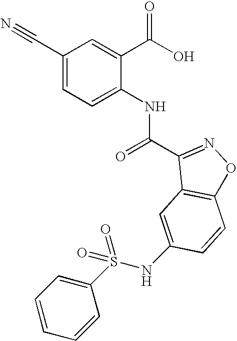 | 0.125 |
| 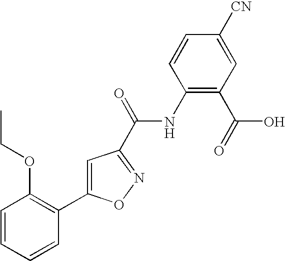 | 0.125 |
| 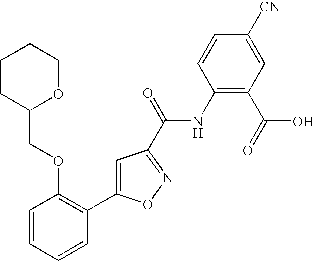 | 2 |
| 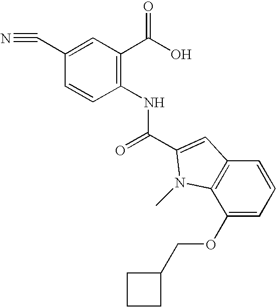 | 0.125 |
| 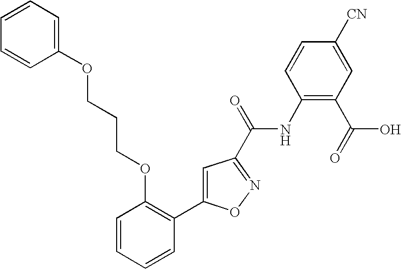 | 0.5 |
| 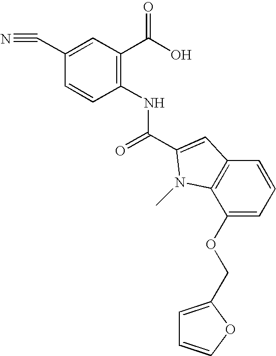 | 0.125 |
| 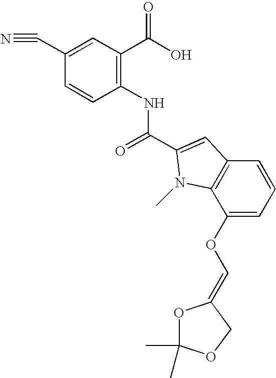 | 0.25 |
| 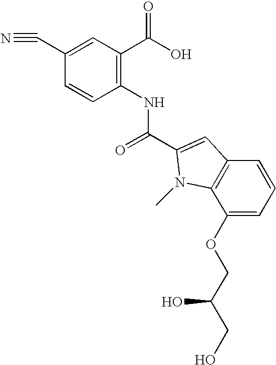 | 0.5 |
| 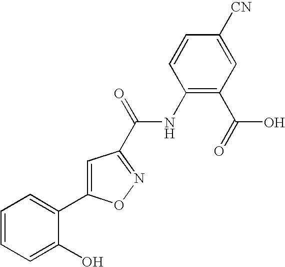 | 0.125 |
| 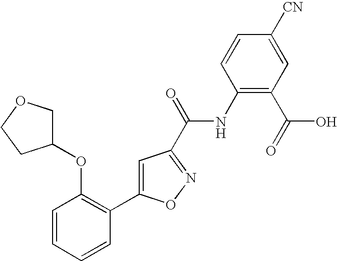 | 0.25 |
| 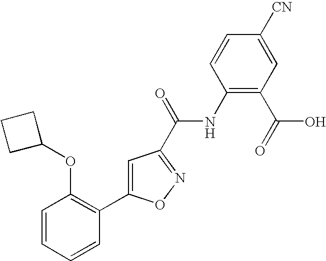 | 0.125 |
| 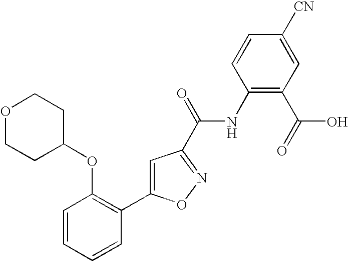 | 0.25 |
| 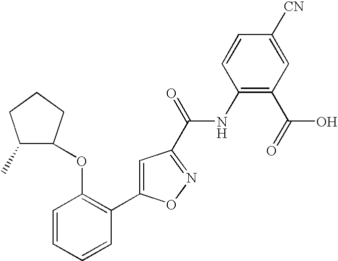 | 0.125 |
| 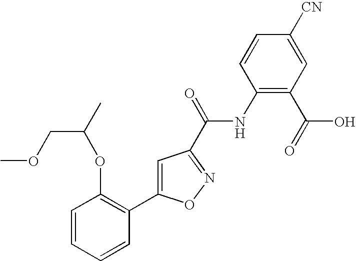 | 0.25 |
| 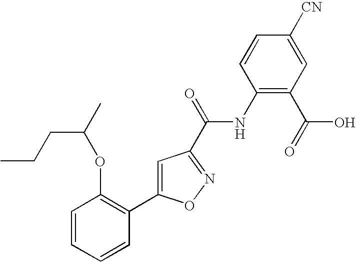 | 0.125 |
| 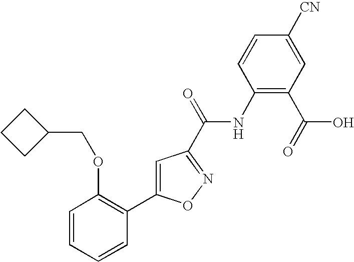 | 0.125 |
| 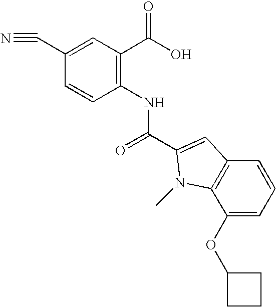 | 0.125 |
| 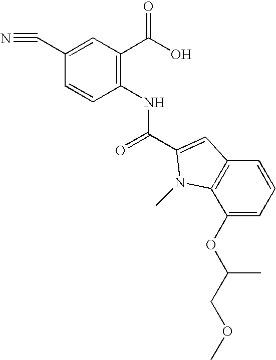 | 0.125 |
| 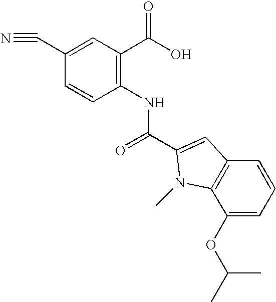 | 0.125 |
| 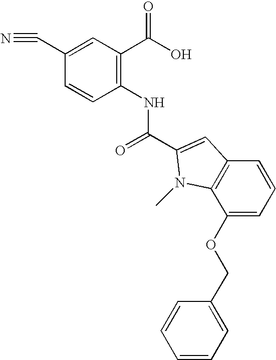 | 0.125 |
| 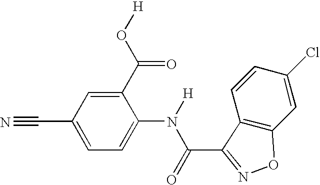 | 1 |
| 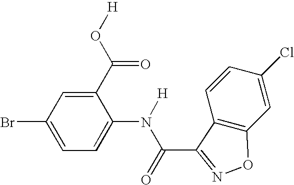 | 1 |
| 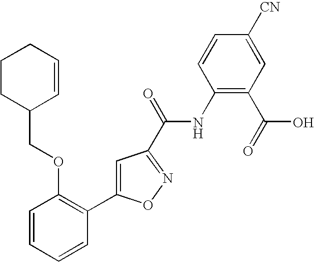 | 2 |
| 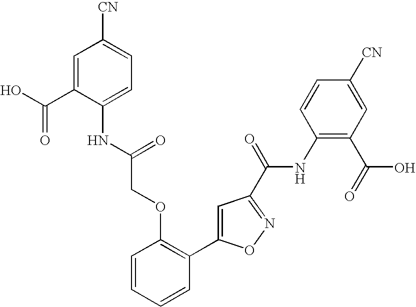 | 128 |
| 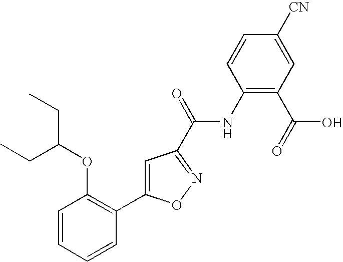 | 4 |
| 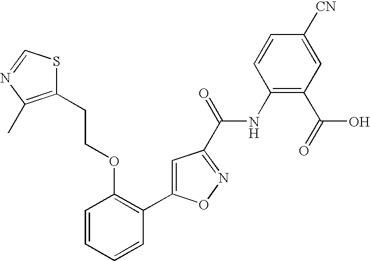 | 128 |
| 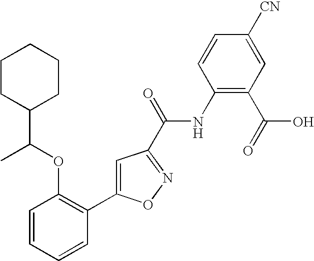 | 1 |
| 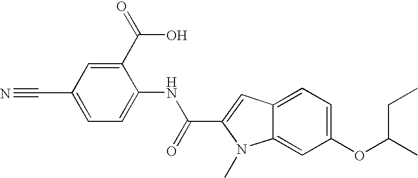 | 16 |
| 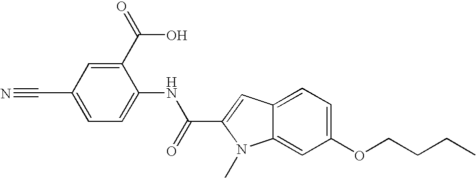 | 16 |
| 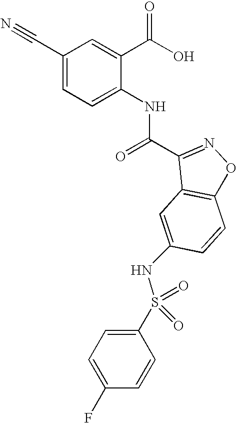 | 0.125 |
| 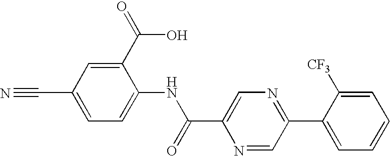 | 32 |
| 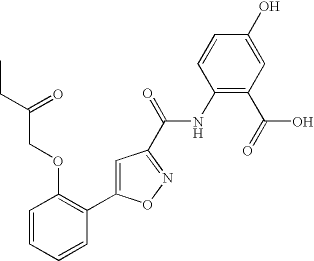 | 1 |
| 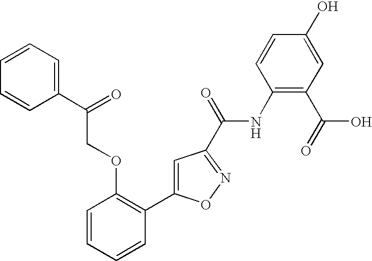 | 0.5 |
| 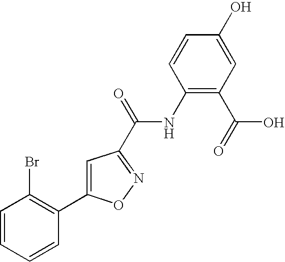 | 0.125 |
| 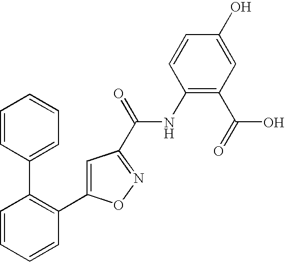 | 0.125 |
| 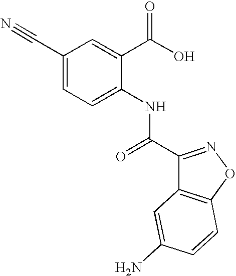 | 0.5 |
| 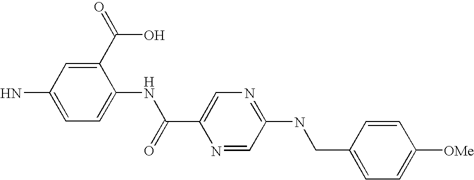 | 0.25 |
| 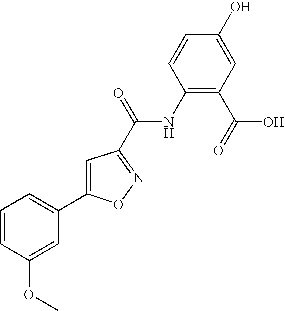 | 0.25 |
| 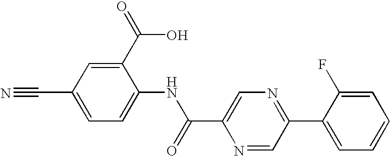 | 4 |
| 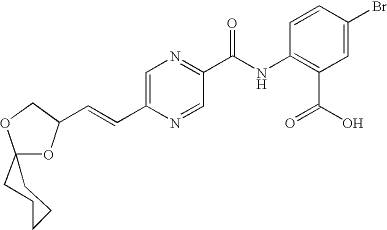 | 8 |
| 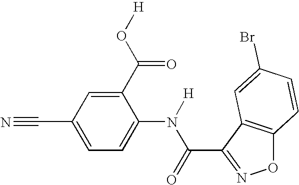 | 0.125 |
| 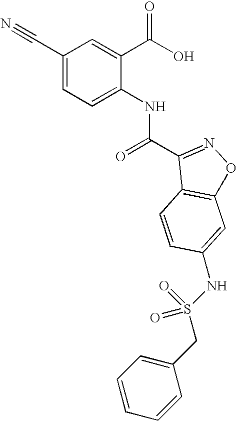 | 1 |
| 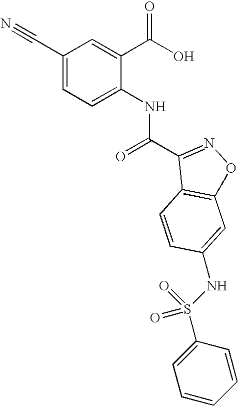 | 1 |
| 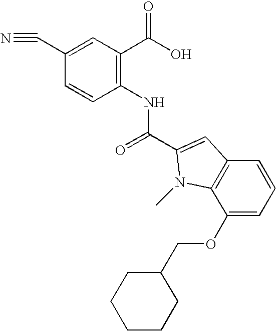 | 0.25 |
| 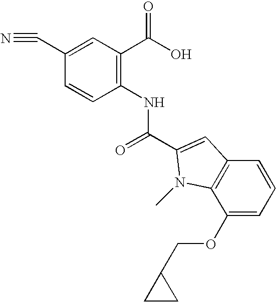 | 0.125 |
| 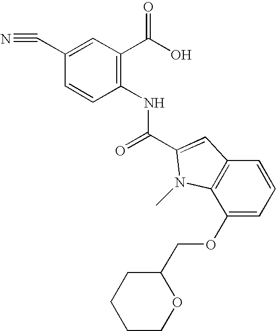 | 0.5 |
| 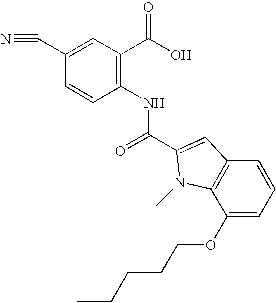 | 0.125 |
| 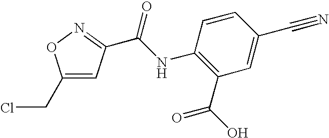 | 4 |
| 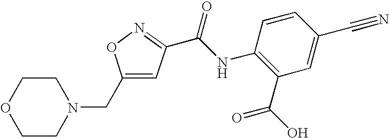 | 16 |
| 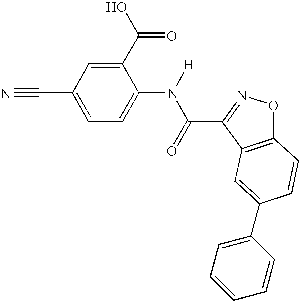 | 0.125 |
| 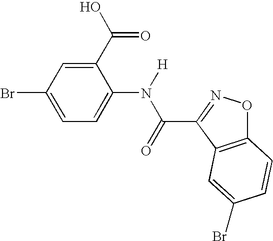 | 1 |
| 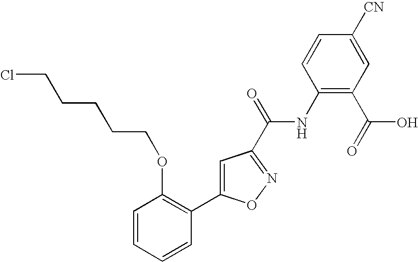 | 0.125 |
| 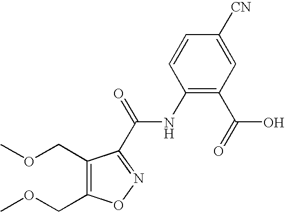 | 16 |
| 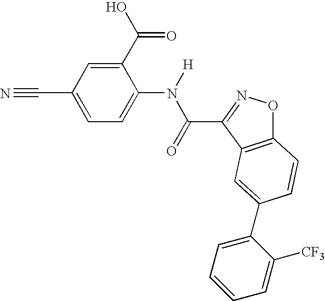 | 0.125 |
| 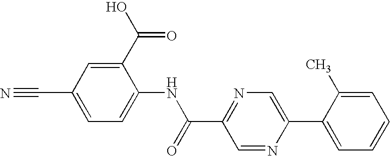 | 32 |
| 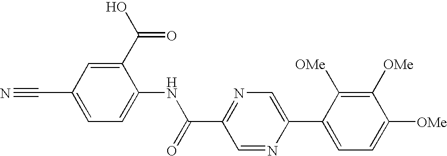 | >128 |
| 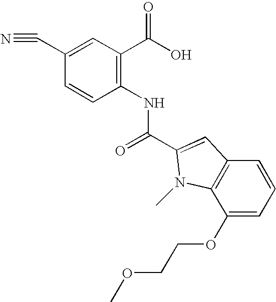 | 0.25 |
| 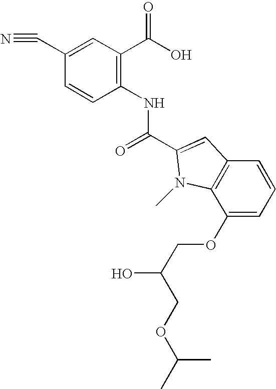 | 0.25 |
| 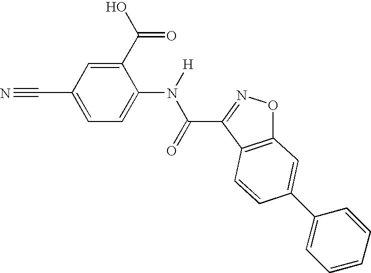 | 0.125 |
| 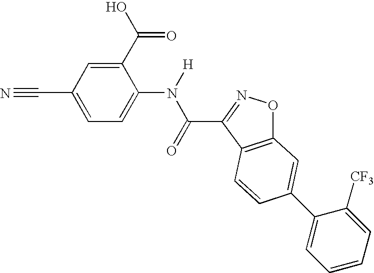 | 0.125 |
| 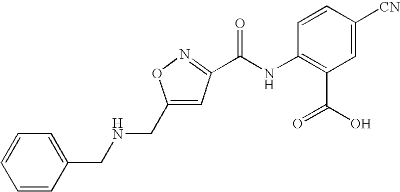 | 2 |
| 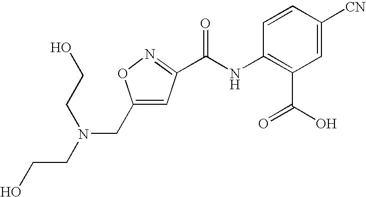 | 8 |
| 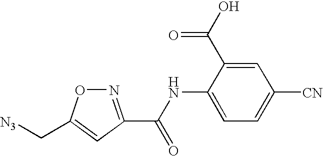 | 4 |
| 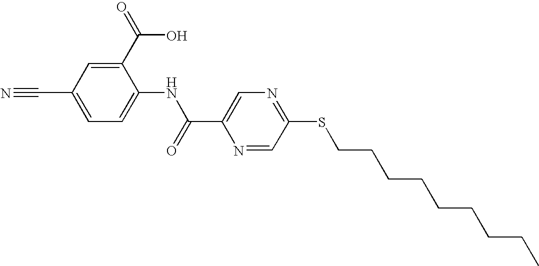 | 2 |
| 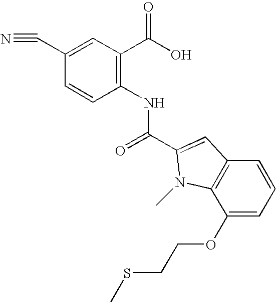 | 0.25 |
| 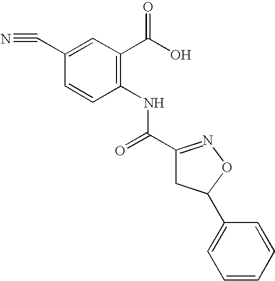 | 4 |
| 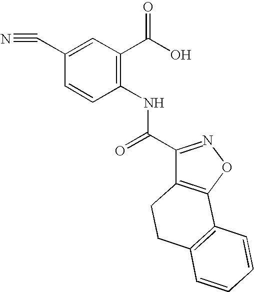 | 0.06 |
| 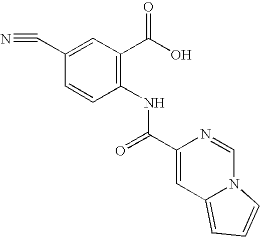 | 32 |
| 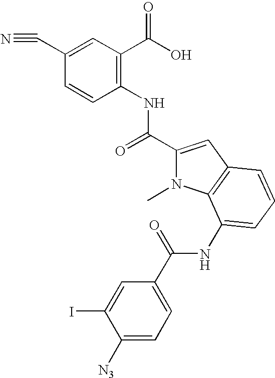 | >128 |
| 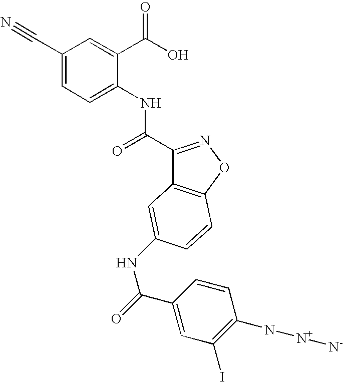 | 64 |
| 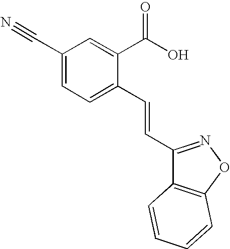 | 64 |
| 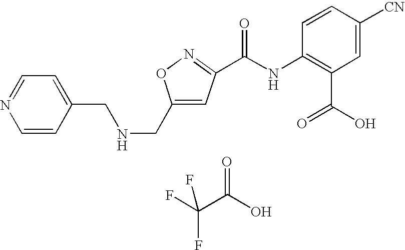 | 8 |
| 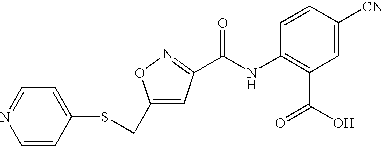 | 1 |
| 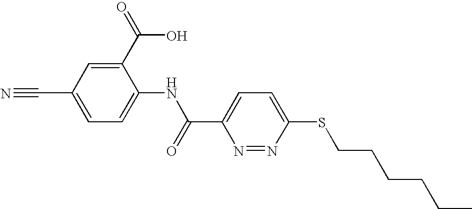 | 0.03 |
| 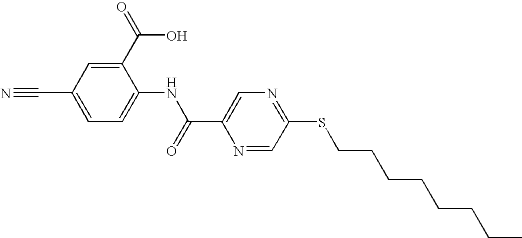 | 0.5 |
| 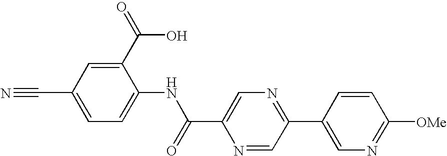 | >128 |
| 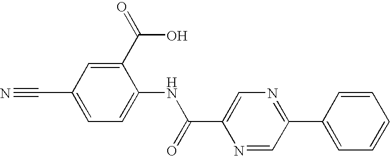 | 64 |
| 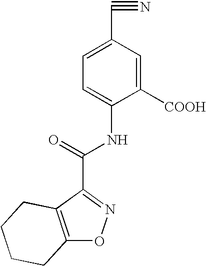 | 0.5 |
| 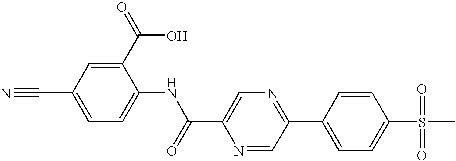 | >128 |
| 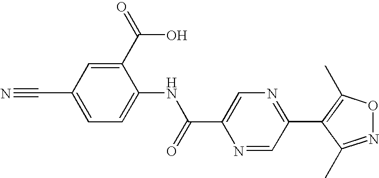 | >128 |
| 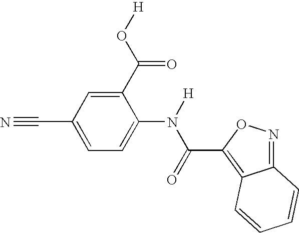 | 1 |
| 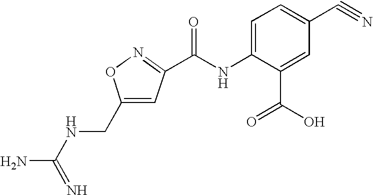 | 128 |
| 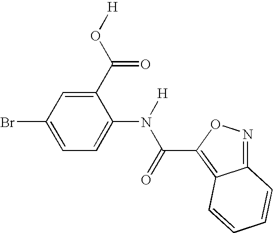 | 2 |
| 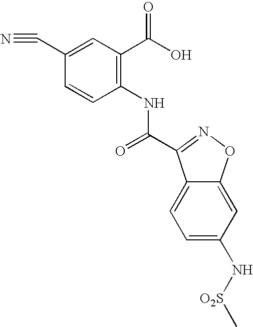 | 2 |
| 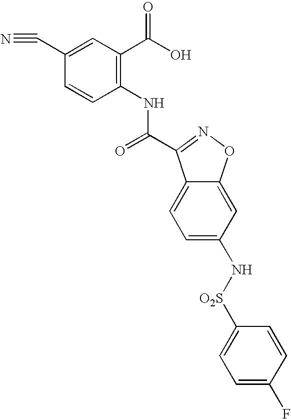 | 0.5 |
| 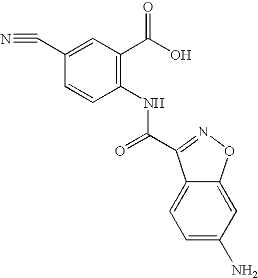 | 0.5 |
| 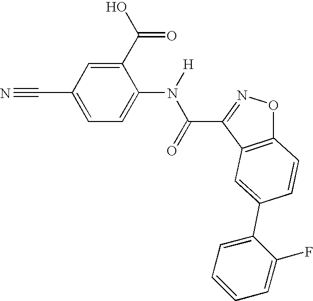 | 0.125 |
| 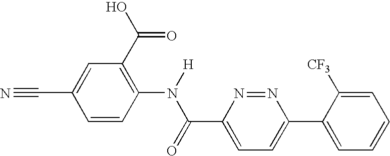 | 16 |
| 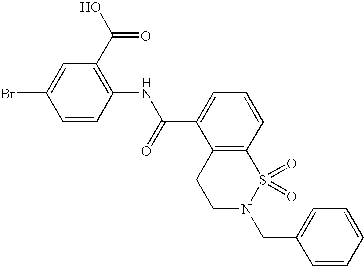 | 8 |
| 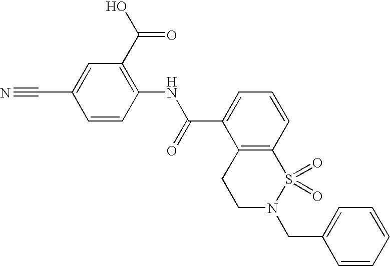 | 8 |
| 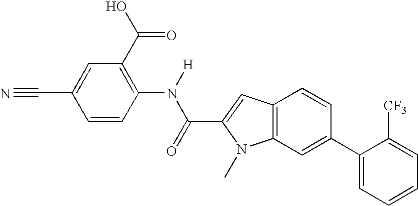 | 0.125 |
| 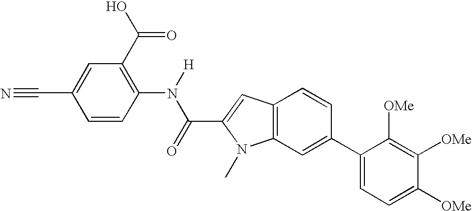 | 0.25 |
| 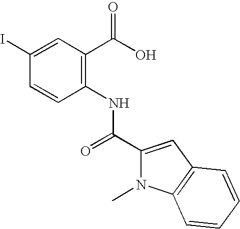 | 4 |
| 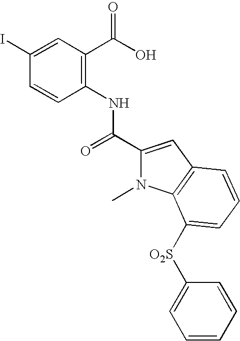 | 0.125 |
| 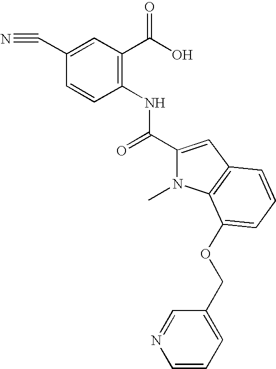 | 0.125 |
| 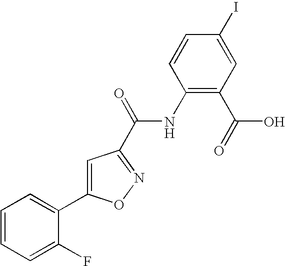 | 0.5 |
| 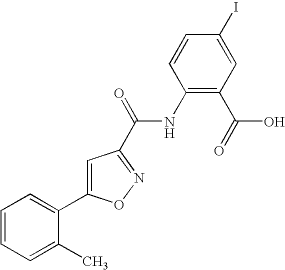 | 0.5 |
| 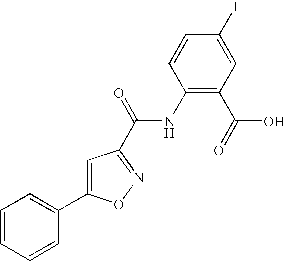 | 1 |
| 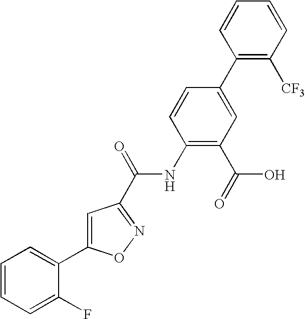 | 8 |
| 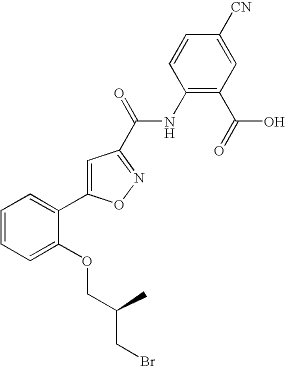 | 0.5 |
| 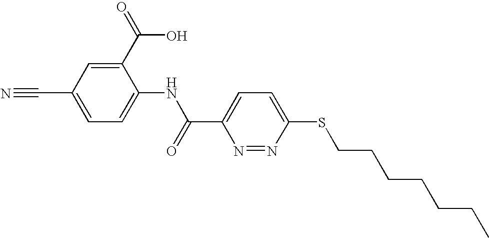 | 0.015 |
| 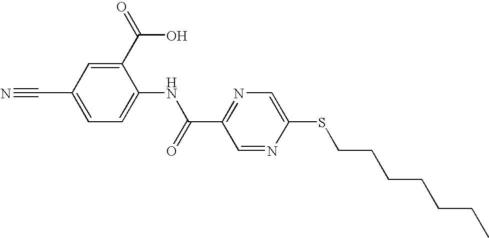 | 0.25 |
| 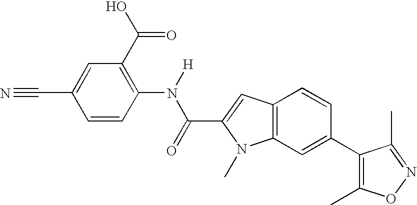 | 1 |
| 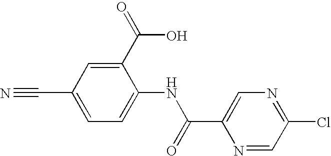 | 32 |
| 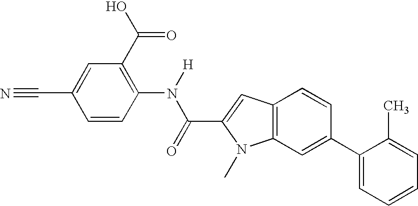 | 0.5 |
| 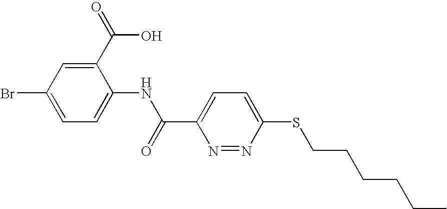 | 0.125 |
| 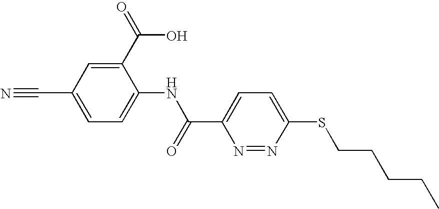 | 0.015 |
| 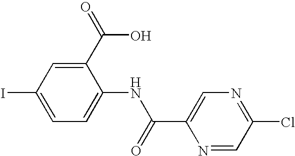 | 128 |
| 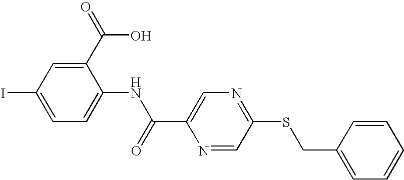 | 1 |
| 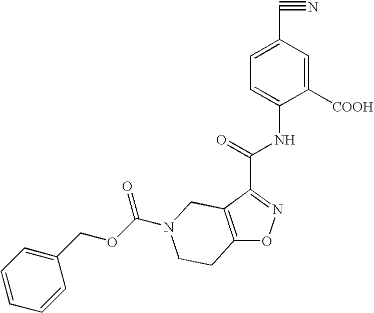 | 0.5 |
| 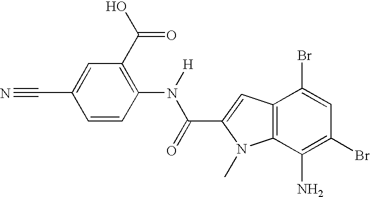 | 0.5 |
| 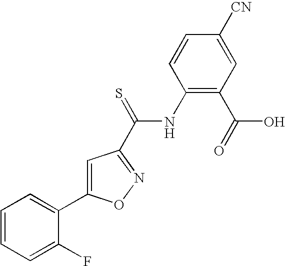 | 0.5 |
| 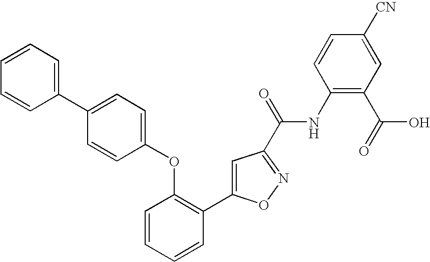 | 16 |
| 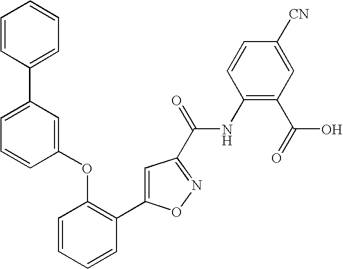 | >128 |
| 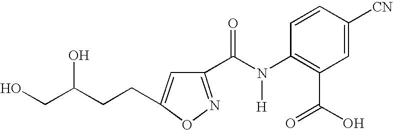 | 8 |
| 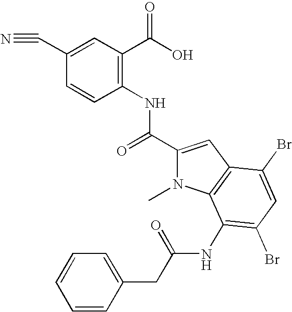 | 2 |
| 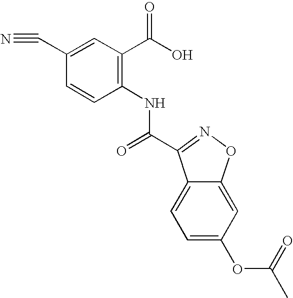 | 1 |
| 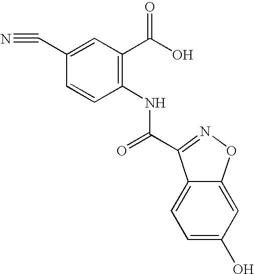 | 1 |
| 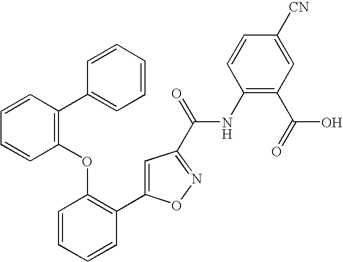 | >128 |
| 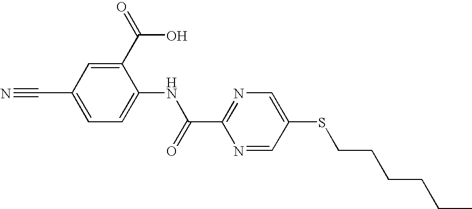 | 2 |
| 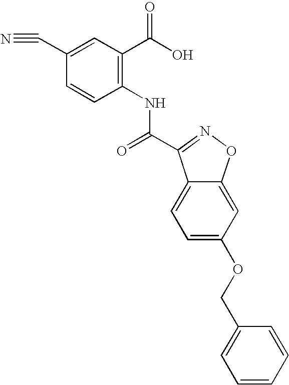 | 0.25 |
| 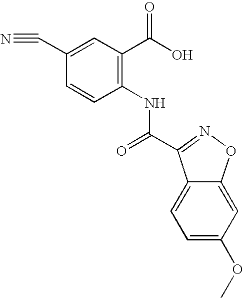 | 0.5 |
| 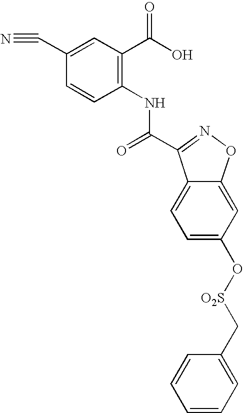 | 0.25 |
| 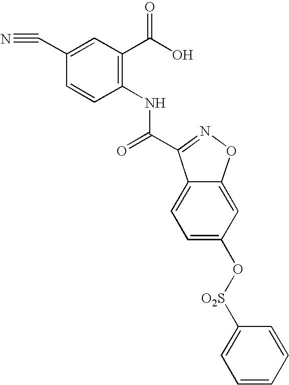 | 0.5 |
| 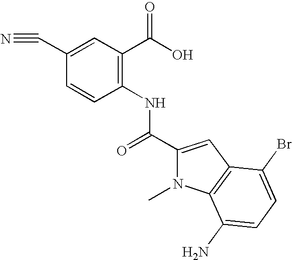 | 1 |
| 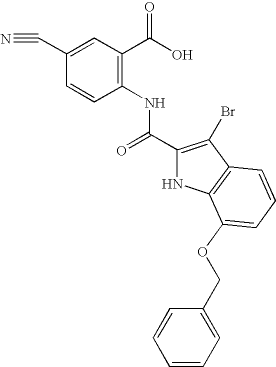 | 0.5 |
| 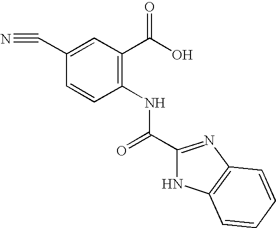 | 4 |
| 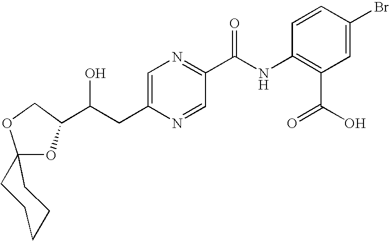 | 128 |
| 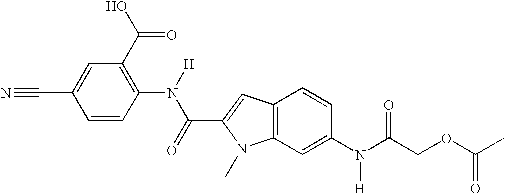 | 1 |
| 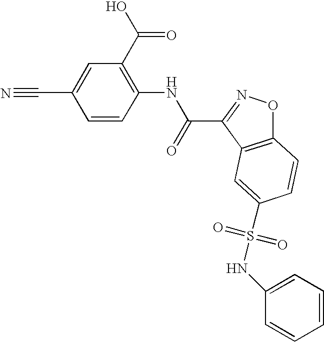 | 0.015 |
| 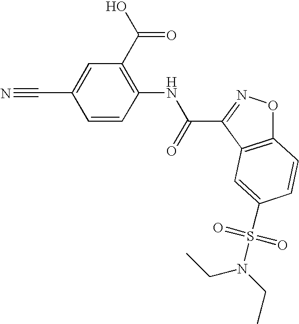 | 0.125 |
| 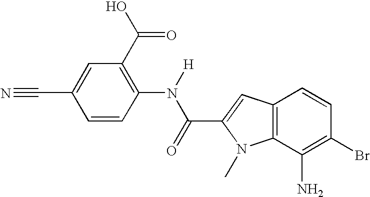 | 0.5 |
| 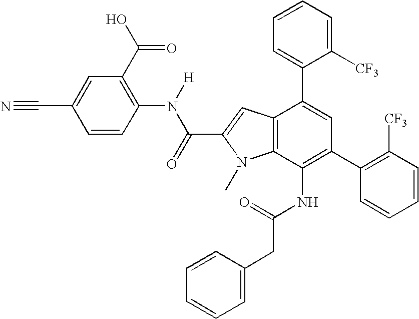 | 8 |
| 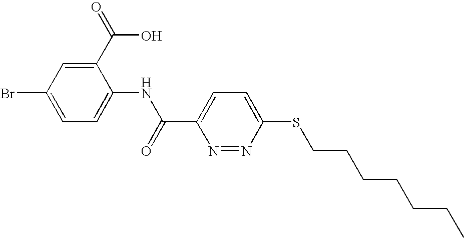 | 0.06 |
| 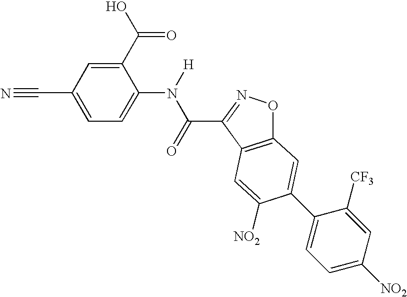 | 0.03 |
| 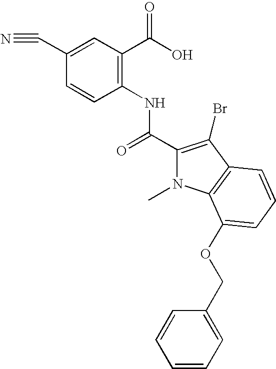 | 1 |
| 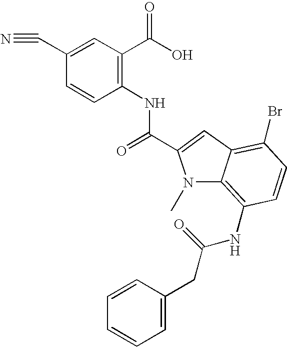 | 2 |
| 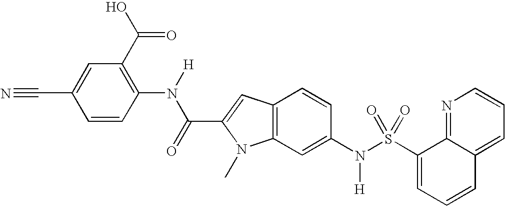 | 1 |
| 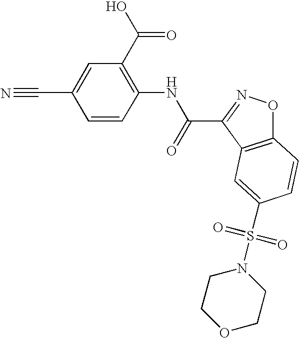 | 0.5 |
| 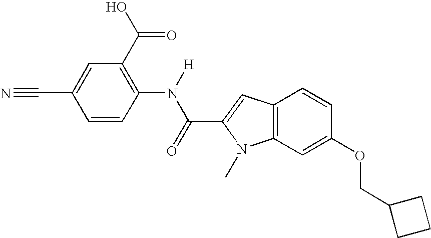 | 0.125 |
| 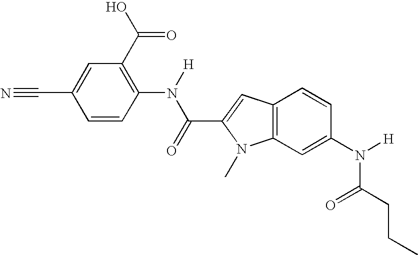 | 1 |
| 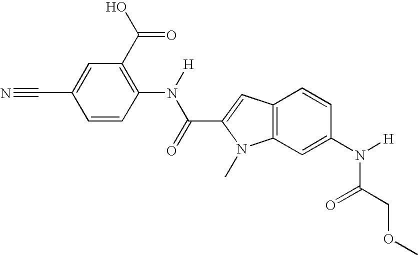 | 2 |
| 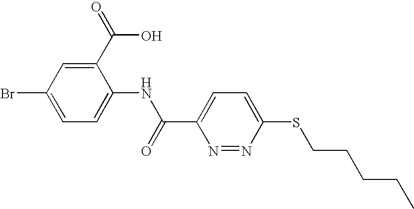 | 0.03 |
| 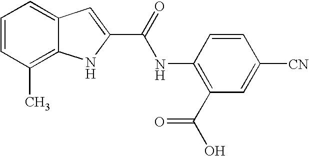 | 2 |
| 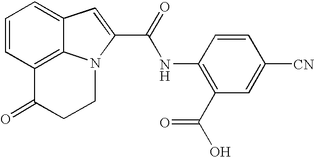 | 1 |
| 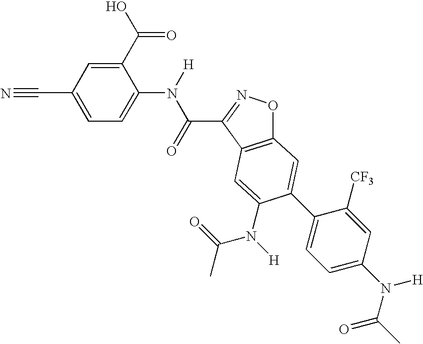 | 0.03 |
| 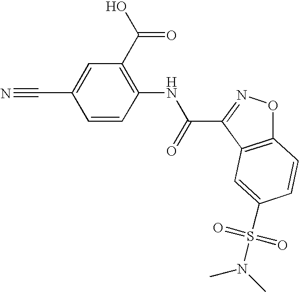 | 0.125 |
| 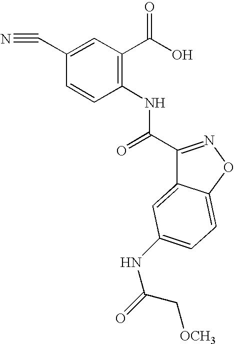 | 4 |
| 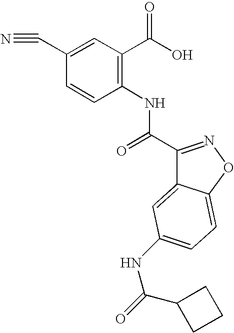 | 2 |
| 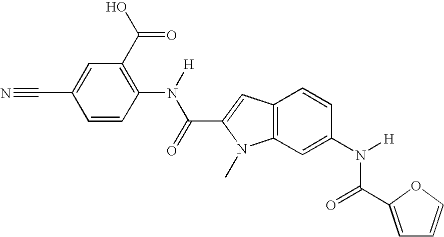 | 2 |
| 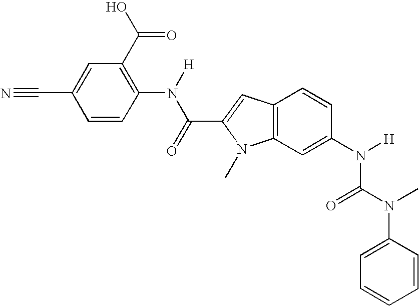 | 0.5 |
| 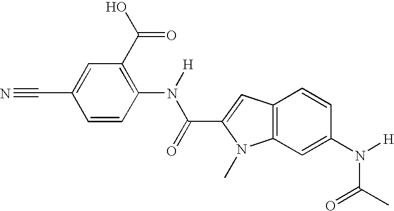 | 1 |
| 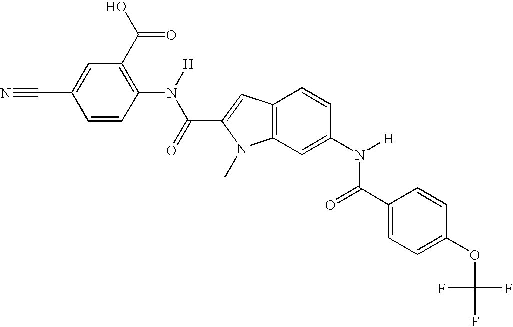 | 2 |
| 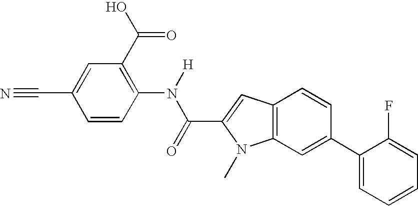 | 0.5 |
| 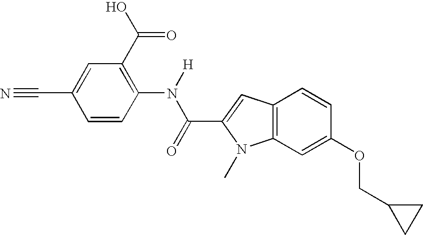 | 0.25 |
| 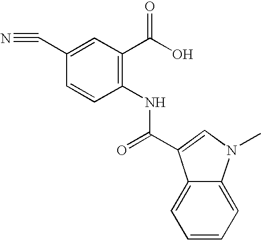 | 16 |
| 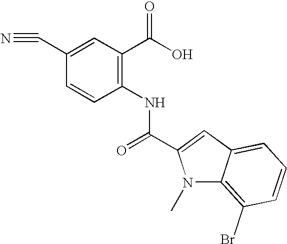 | 0.5 |
| 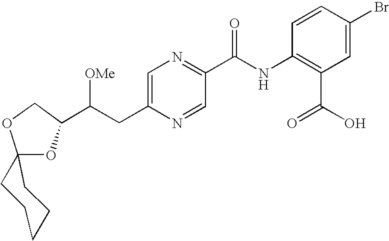 | 128 |
| 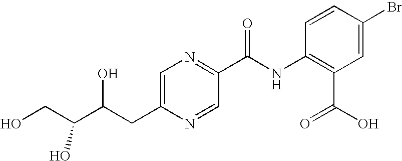 | >128 |
| 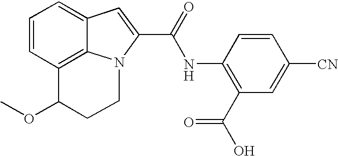 | 0.5 |
| 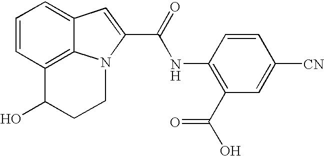 | 0.5 |
| 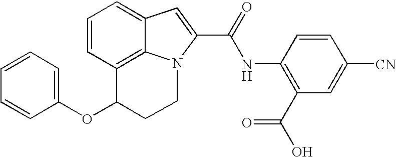 | 0.25 |
| 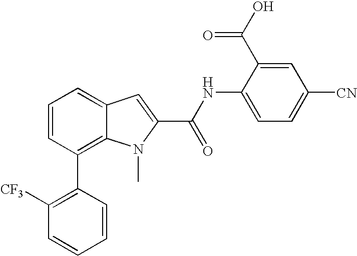 | 0.06 |
| 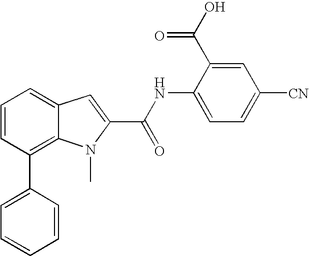 | 0.25 |
| 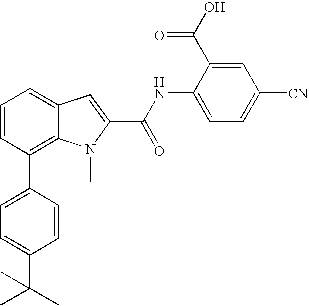 | 4 |
| 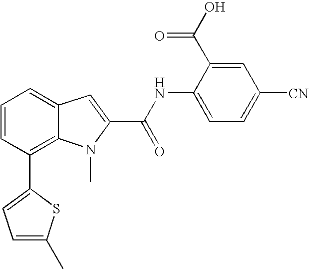 | 0.5 |
| 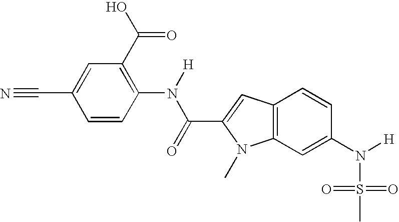 | 4 |
| 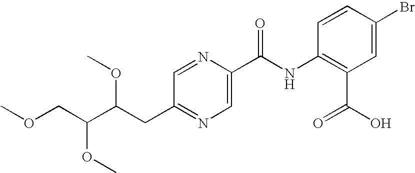 | >128 |
| 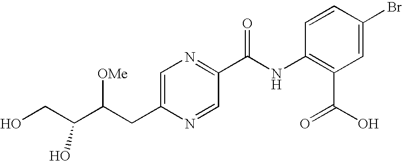 | >128 |
| 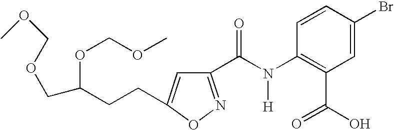 | 8 |
| 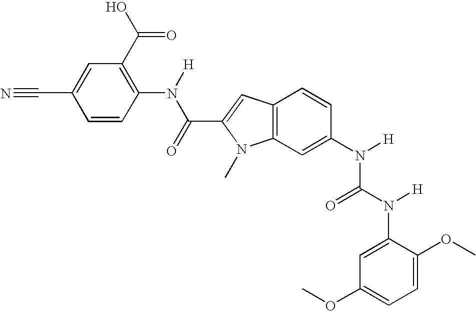 | 2 |
| 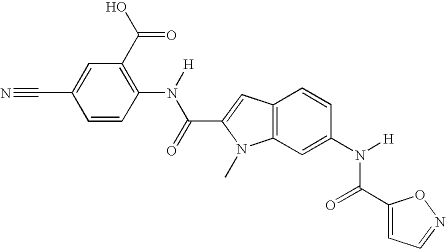 | 0.5 |
| 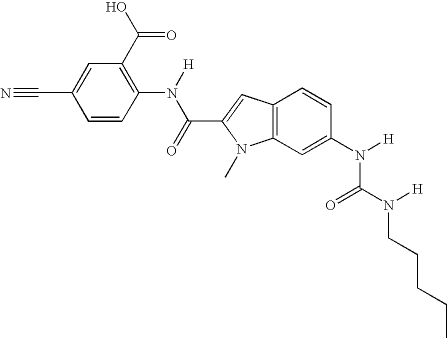 | 0.5 |
| 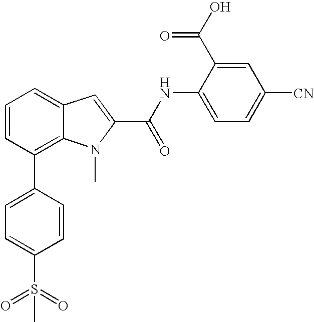 | 8 |
| 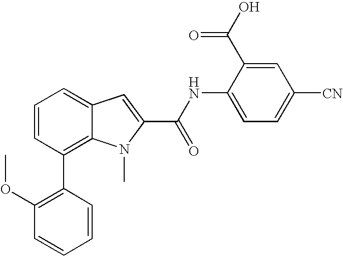 | 0.25 |
| 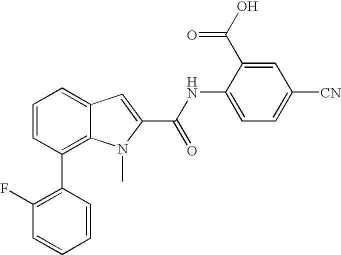 | 0.25 |
| 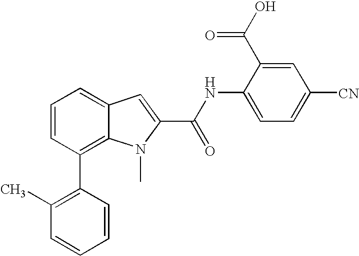 | 0.125 |
| 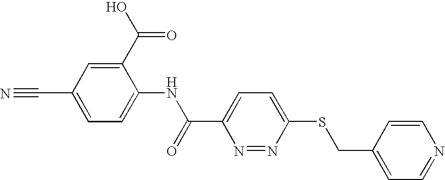 | 0.25 |
| 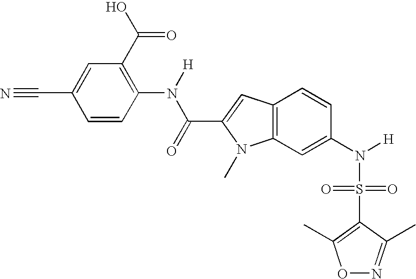 | 2 |
| 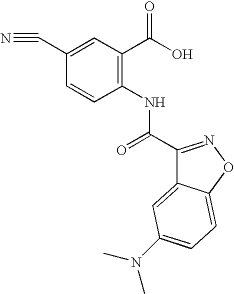 | 0.25 |
| 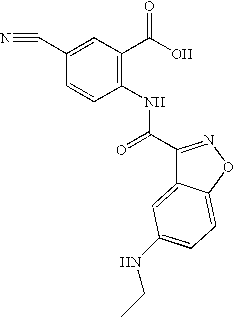 | 0.5 |
| 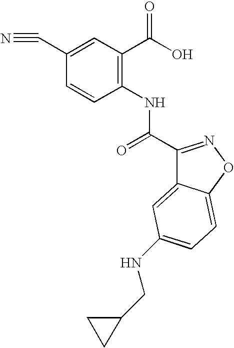 | 0.5 |
| 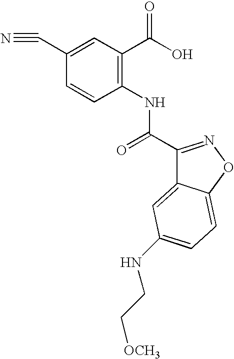 | 1 |
| 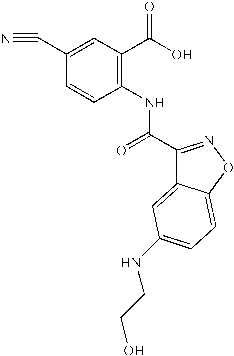 | 1 |
| 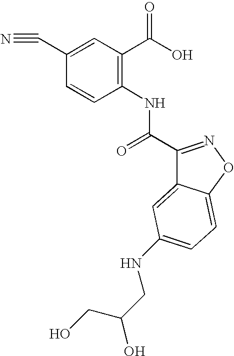 | 2 |
| 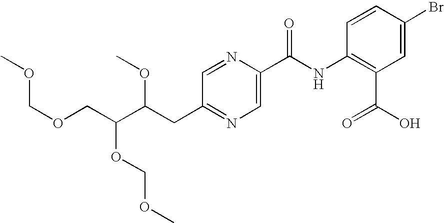 | >128 |
| 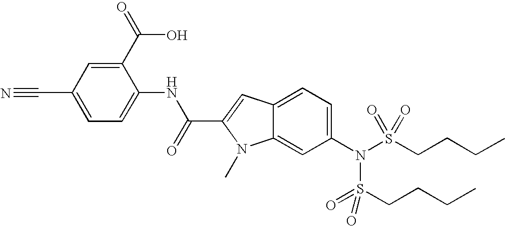 | 2 |
| 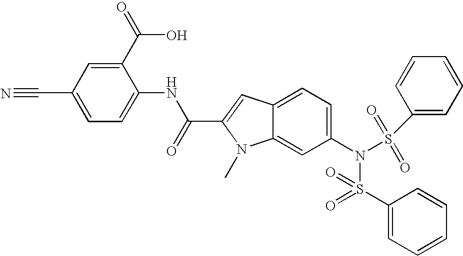 | 0.5 |
| 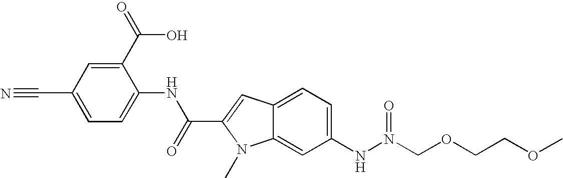 | 0.5 |
| 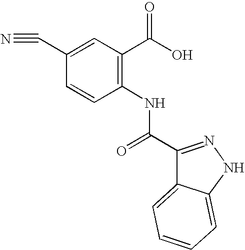 | 16 |
| 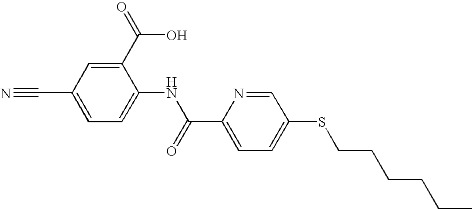 | 0.125 |
| 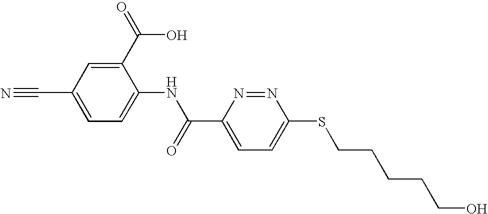 | 0.06 |
| 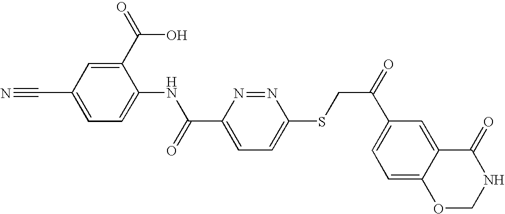 | 0.5 |
| 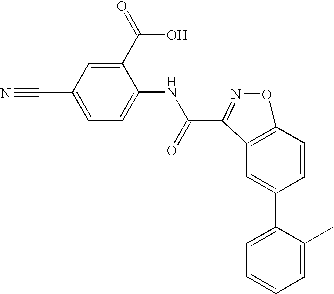 | 0.125 |
| 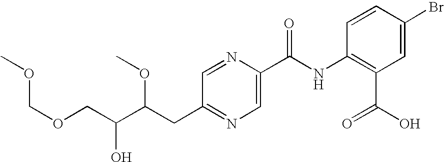 | 128 |
| 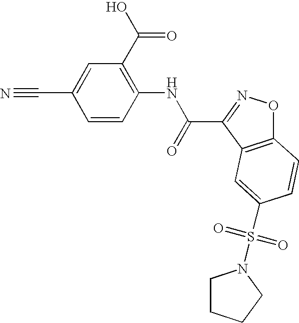 | 0.06 |
| 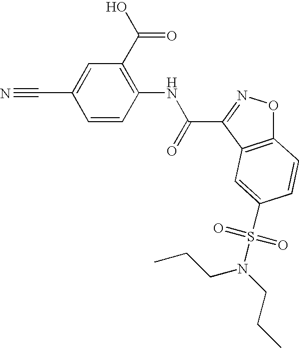 | 0.25 |
| 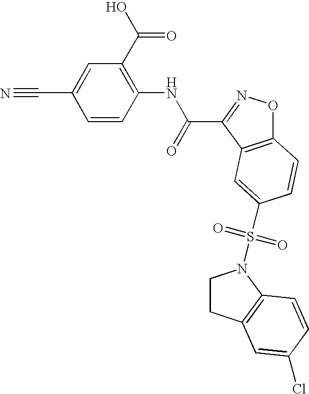 | 0.03 |
| 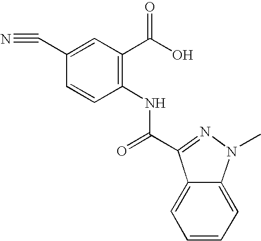 | 32 |
| 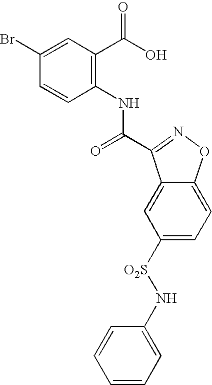 | 0.03 |
| 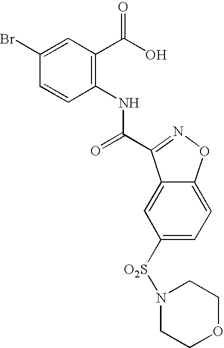 | 0.5 |
| 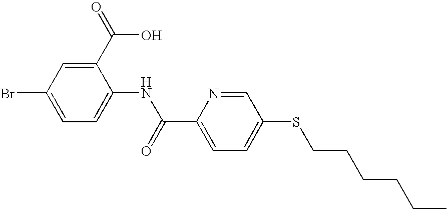 | 0.5 |
| 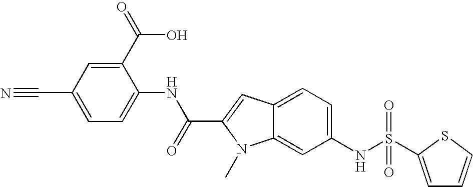 | 0.5 |
| 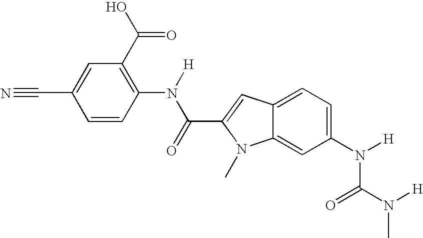 | 2 |
| 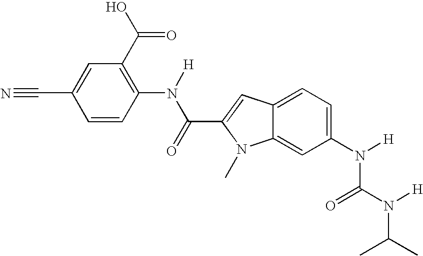 | 0.5 |
| 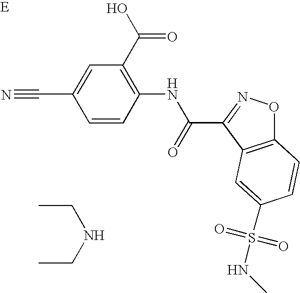 | 0.06 |
| 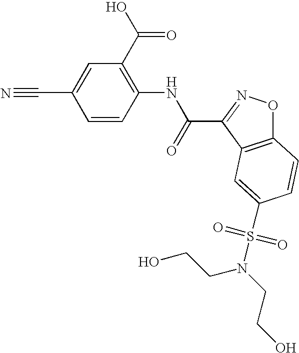 | 0.5 |
| 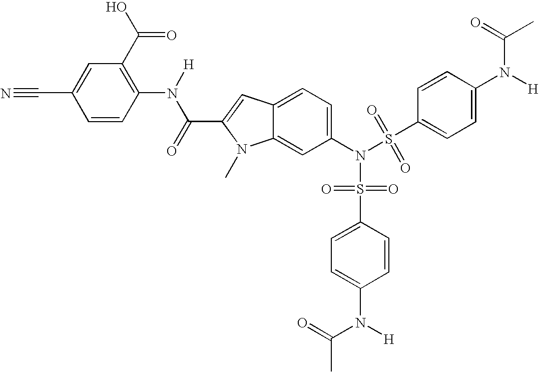 | 1 |
| 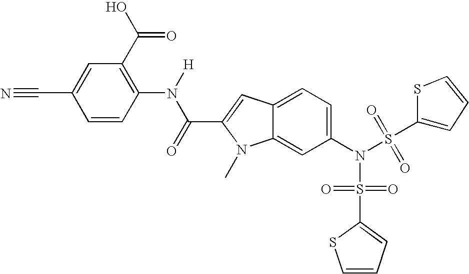 | 0.06 |
| 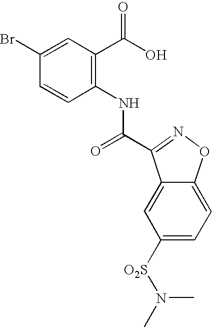 | 0.25 |
| 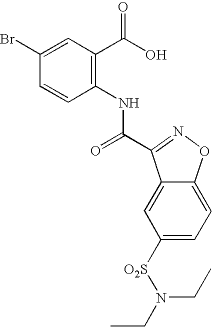 | 0.25 |
| 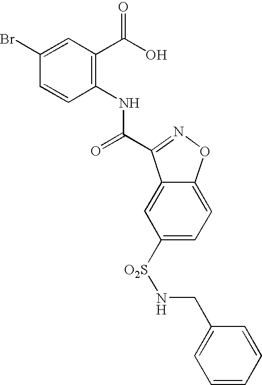 | 0.06 |
| 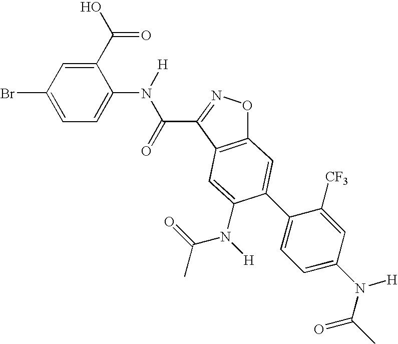 | 0.0075 |
| 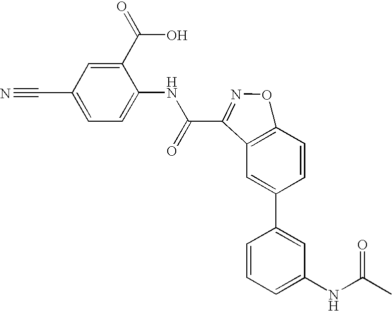 | 0.5 |
| 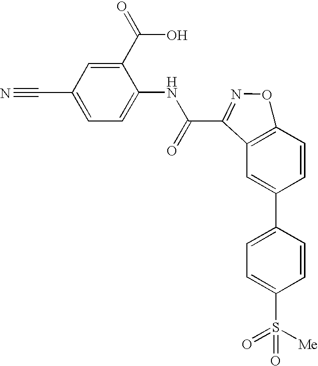 | 0.5 |
| 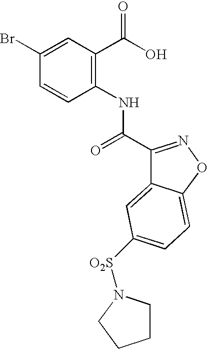 | 0.25 |
| 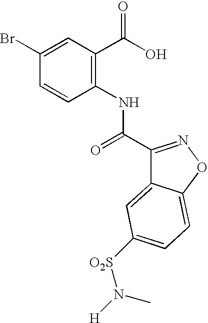 | 0.125 |
| 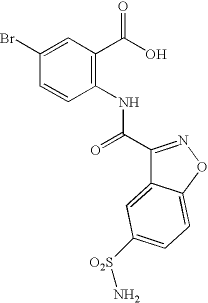 | 0.25 |
| 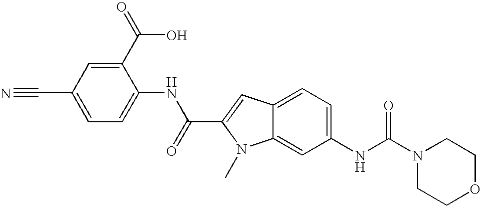 | 4 |
| 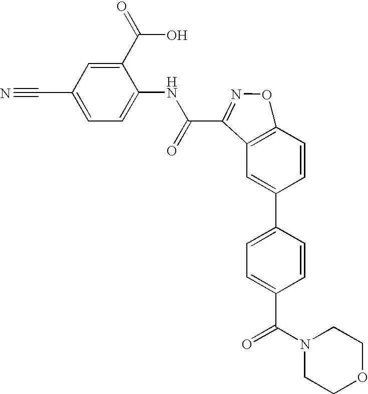 | 0.25 |
| 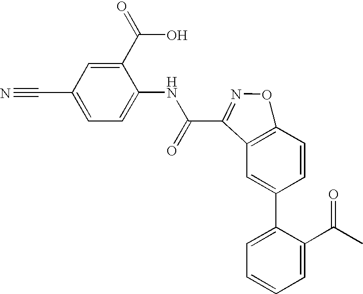 | 0.5 |
| 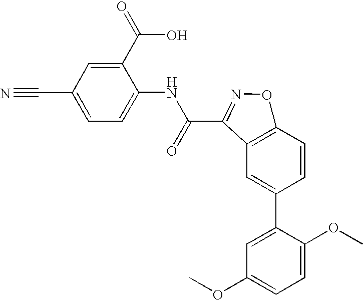 | 0.25 |
| 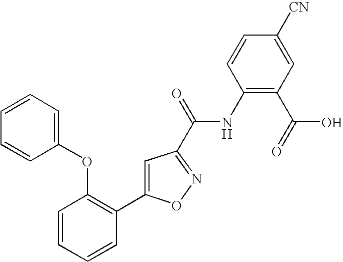 | 0.03 |
| 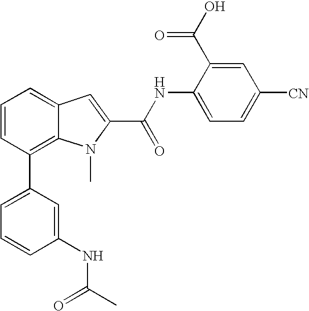 | 0.5 |
| 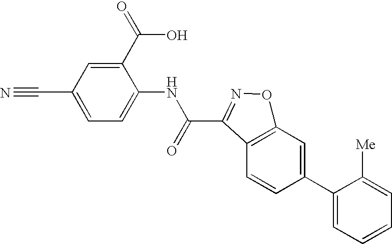 | 0.06 |
| 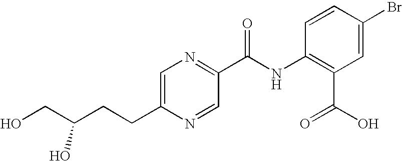 | 64 |
| 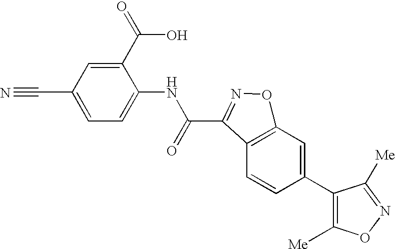 | 0.125 |
| 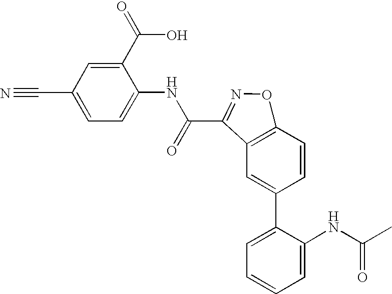 | 0.5 |
| 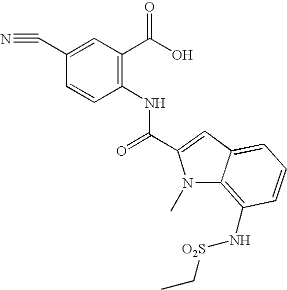 | 0.5 |
| 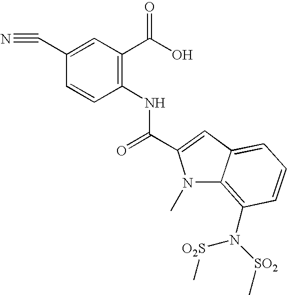 | 0.125 |
| 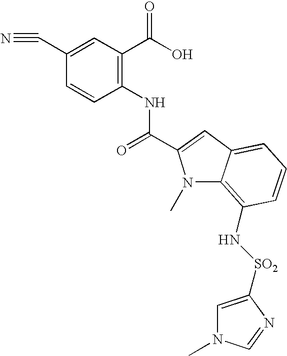 | 0.5 |
| 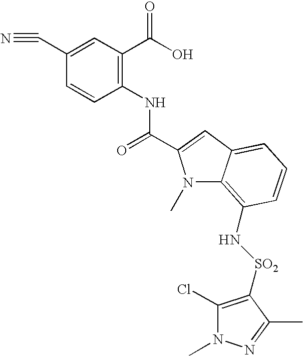 | 0.125 |
| 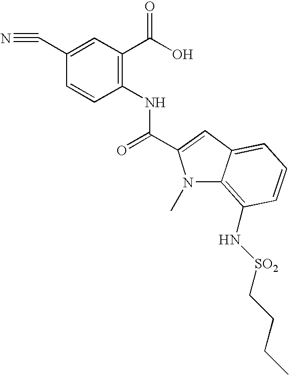 | 0.25 |
| 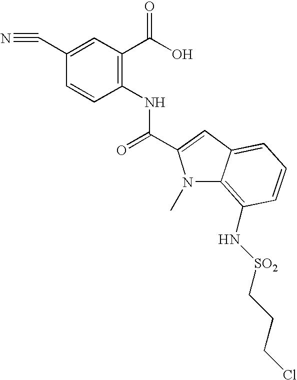 | 0.125 |
| 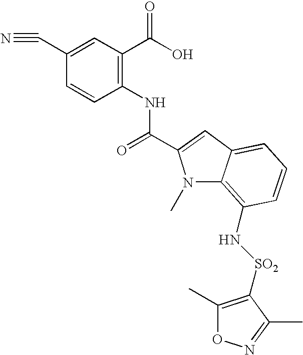 | 0.25 |
| 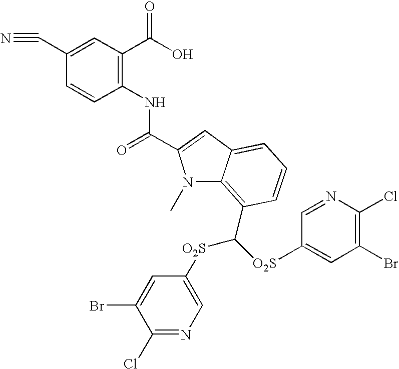 | 0.03 |
| 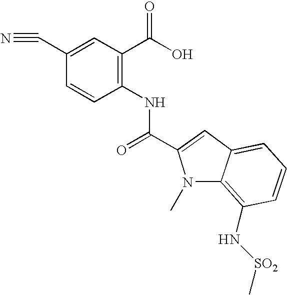 | 0.5 |
| 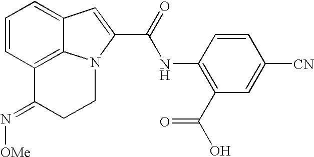 | 1 |
| 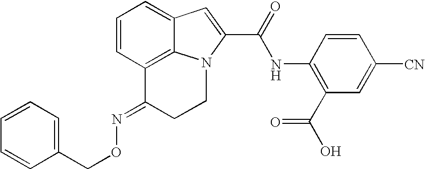 | 0.5 |
| 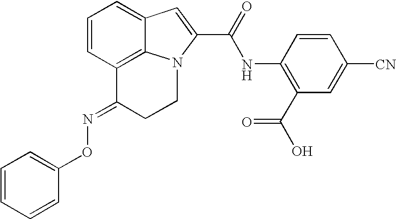 | 2 |
| 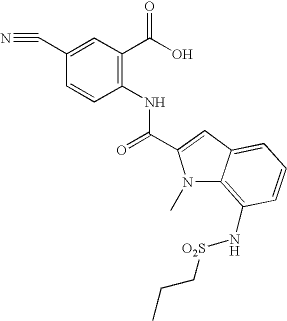 | 0.5 |
| 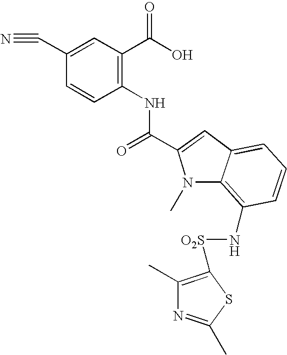 | 0.5 |
| 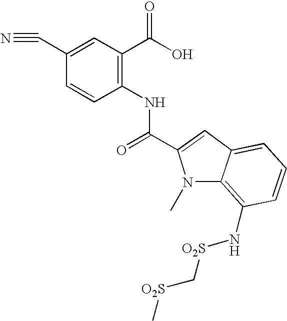 | 2 |
| 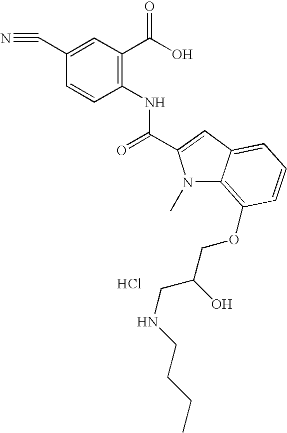 | 0.5 |
| 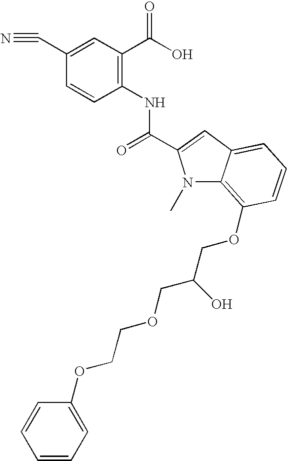 | 0.5 |
| 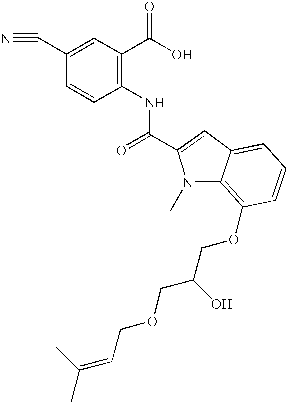 | 0.5 |
| 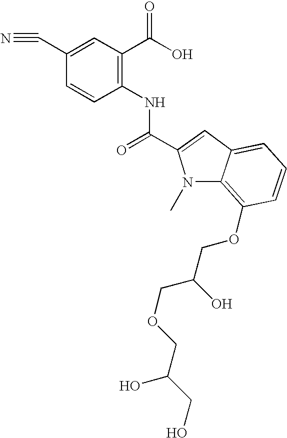 | 2 |
| 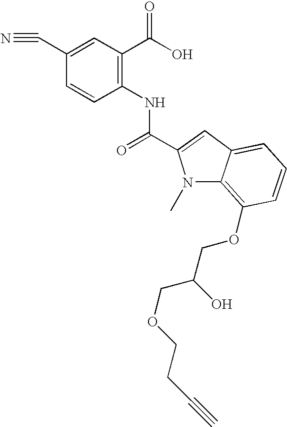 | 0.5 |
| 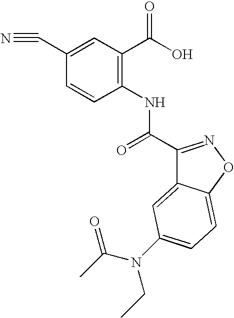 | 0.25 |
| 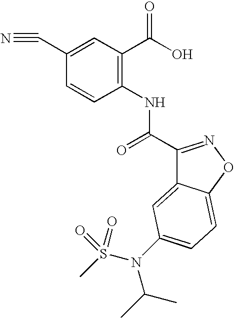 | 0.125 |
| 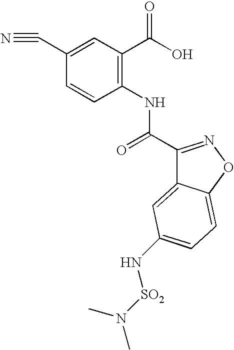 | 0.5 |
| 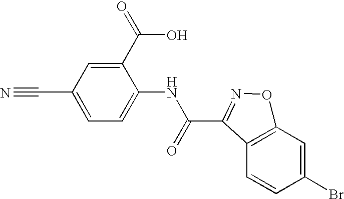 | 0.5 |
| 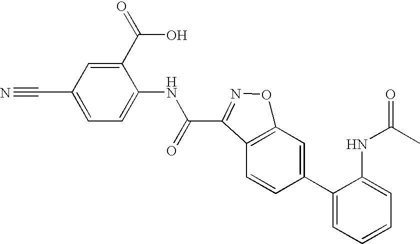 | 0.5 |
| 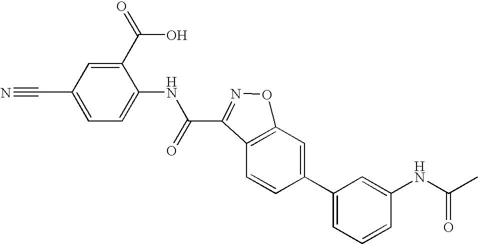 | 0.015 |
| 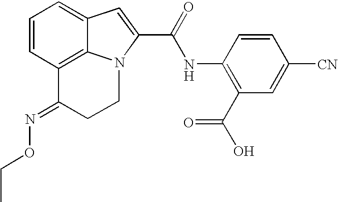 | 1 |
| 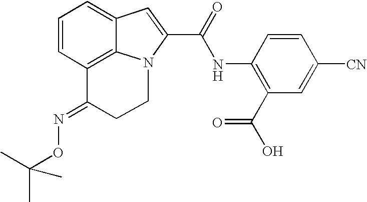 | 1 |
| 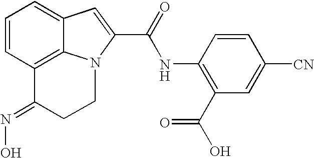 | 0.25 |
| 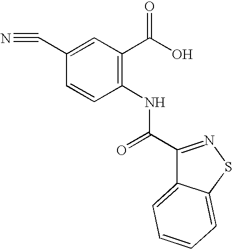 | 1 |
| 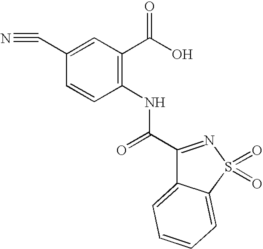 | >128 |
| 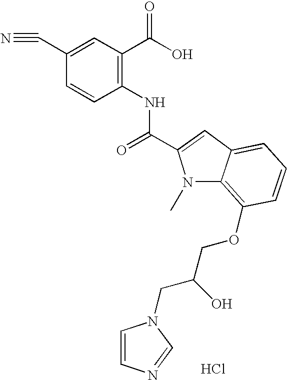 | 0.5 |
| 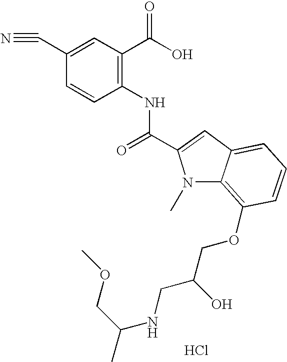 | 1 |
| 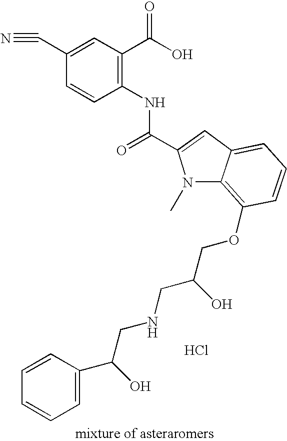 | 1 |
| 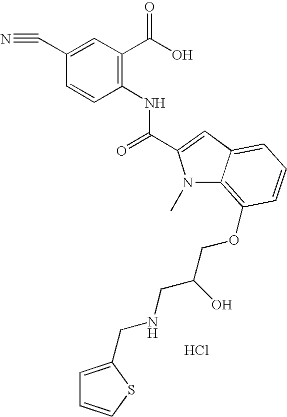 | 0.5 |
| 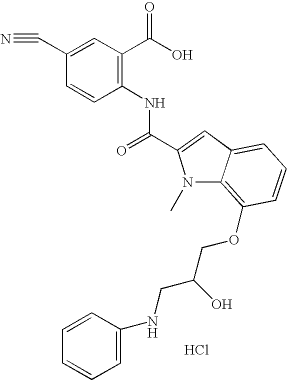 | 0.125 |
| 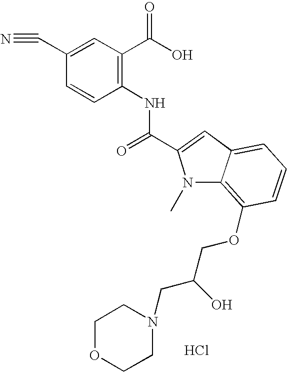 | 1 |
| 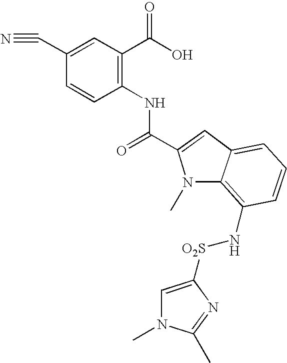 | 1 |
| 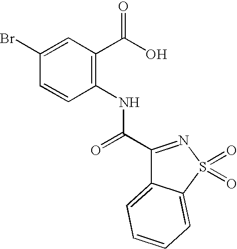 | >128 |
| 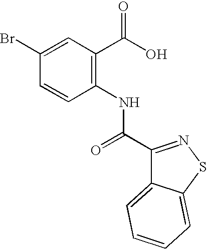 | 1 |
| 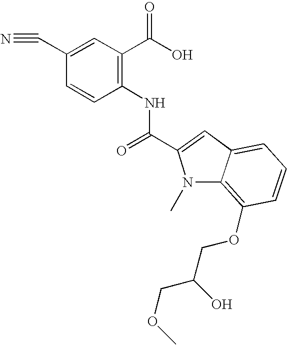 | 0.5 |
| 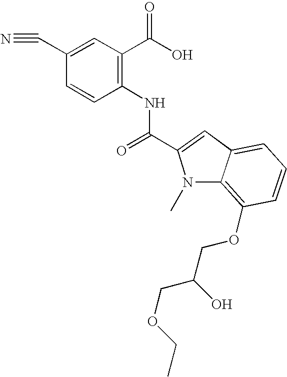 | 0.25 |
| 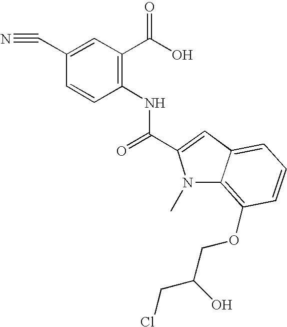 | 0.25 |
| 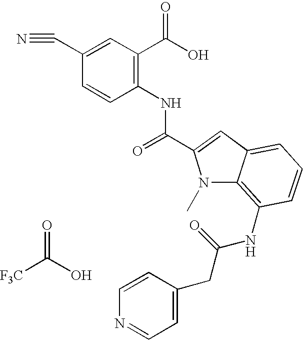 | 1 |
| 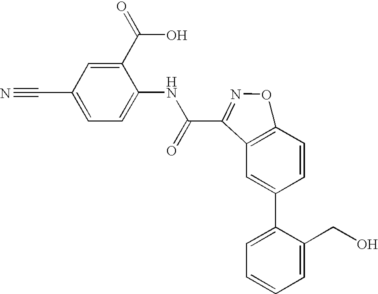 | 0.5 |
| 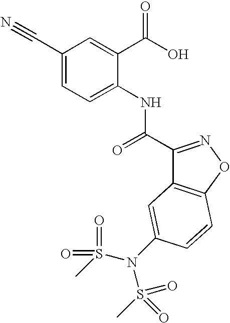 | 0.125 |
| 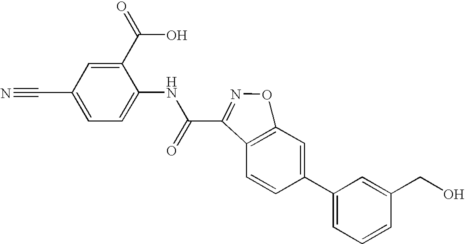 | 0.125 |
| 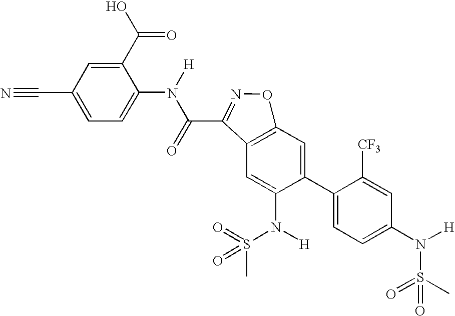 | 0.125 |
| 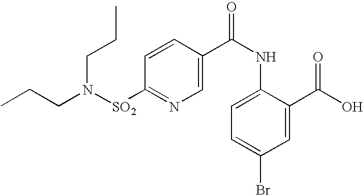 | 8 |
| 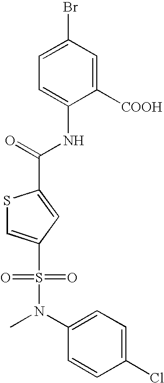 | 8 |
| 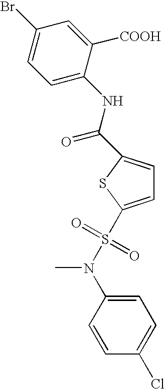 | 2 |
| 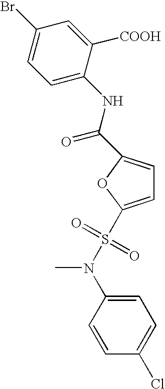 | 16 |
| 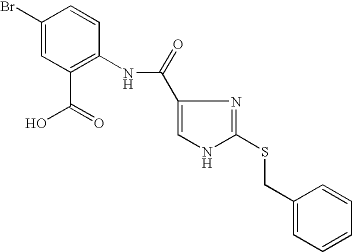 | >128 |
| 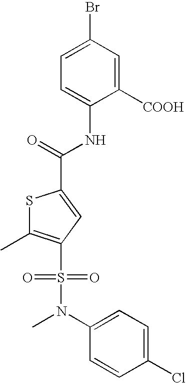 | 2 |
| 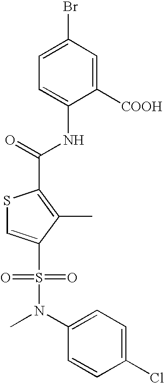 | 8 |
| 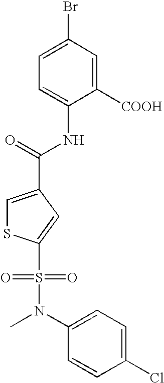 | 4 |
| 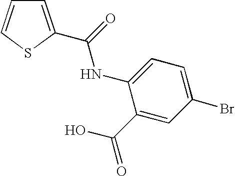 | >128 |
| 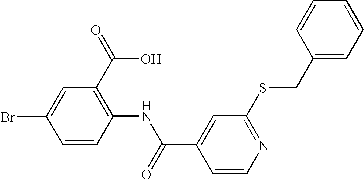 | 2 |
| 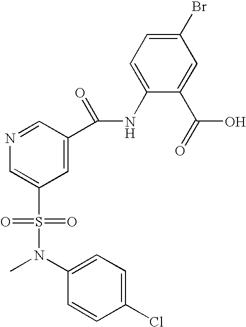 | 1 |
| 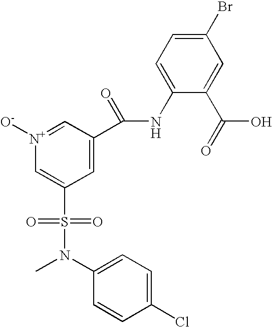 | 8 |
| 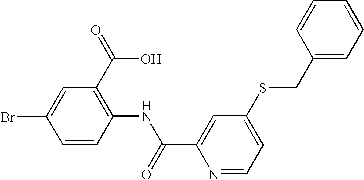 | 2 |
| 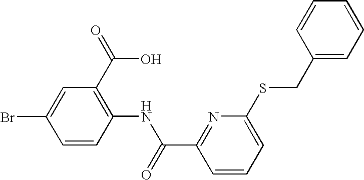 | 16 |

What is claimed is:

1. A compound of formula I, 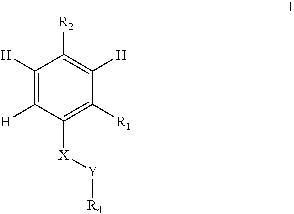
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group; and
R₄ is an optionally substituted HET, provided that the HET is not simultaneously substituted with a sulfonamide and a urea or thiourea.

2. The compound of claim 1 having a formula II 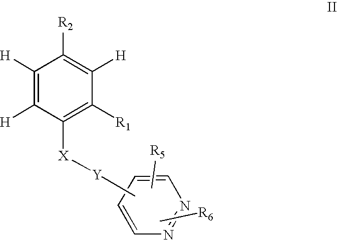
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is —(CH₂)ₖ—S(O)ᵢ-R₇, —NH—SO₂—R₇, —(CH₂)ₖ-W-R₈, —NH—(CZ₂)—R₈, —NH—(CZ₁)—NR₈, substituted aryl, substituted C₁₋₄alkyl, or substituted C₁₋₄alkenyl;
R₆ is selected from H. halo, HET, —CN, NH2, NO₂, alkyl, substituted alkyl, alkoxy, substituted alkoxy, —NH—CO-HET, and —NH—CO-aryl;
R₇ is selected from alkyl, substituted alkyl, aryl, substituted aryl, -N(QI₅)₂, HET, and substituted HET;
F₈ is H, alkyl, substituted alkyl, aryl, substituted aryl, HET, substituted HET, cycloalkyl, substituted cycloalkyl;
Each QI5 is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═—S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆SQ₁₆, —NO₂, and —SNQ₁₆Q₁₆. The alkyl, cycloalkyl, and cycloalkenyl being furher optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is 0;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

3. The compound of claim 1 having a formula III 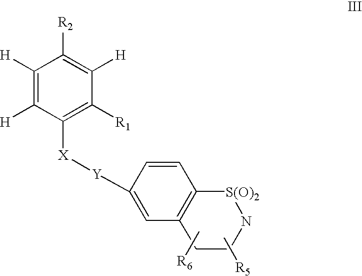
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is CH₂)ₖ-S(O)ᵢ-R₇, —NH—SO₂—R₇, —(CH₂)ₖ-W-R₈, —NH—(CZ₁)—R₈, —NH—(CZ₁)—NR₈, substituted aryl, substituted C₁₋₄alyl, or substituted C₁₋₄alenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q_{16,} —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, |—C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is O;
Z₂ is ═O, ═S , ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

4. The compound of claim 1 having a formula IV 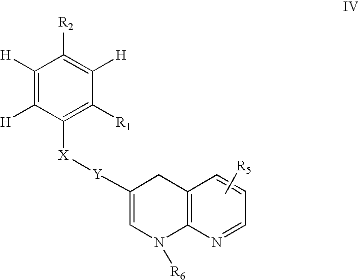
or a pharmaceutically acceptable salt thereof,
wherein
X═NH
Y═CO, CS, —C(═N—CN) or
X and Y together form an alkene, or C₃-C₅ cycloalkyl;
R₁ is —COOH,
R₂ is an electron withdrawing group;
R₅ is CH₂)ₖ-S(O)ᵢ-R₇, —NH—SO₂-R₇, —(CH₂)ₖ-W-R₈, —NH—(CZ₁)—R₈, —NH(CZ₁)-R₈, —NH—(CZ₁)—NR₈, substituted aryl, substituted C₁₄alkyl, or substituted C₁₋₄alkenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆,—NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z, is 0;
Z₂ is ═O, ═S , ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

5. The compound of claim 1 having a formula V 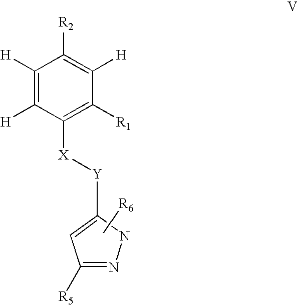
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is —(CH₂)ₖ-S(O)ᵢ-R₇, —NH—SO₂-R₇, —(CH₂)ₖ-W-R₈, —NH—(CZ₁)-R₈, —NH(CZ₁)—NR₈, substituted aryl, substituted C₁₋₄alyl, or substituted C₁₋₄alkenyl;
P6 is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is O;
Z₂ is ═O, ═S , ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

6. The compound of claim 1 having a formula XX 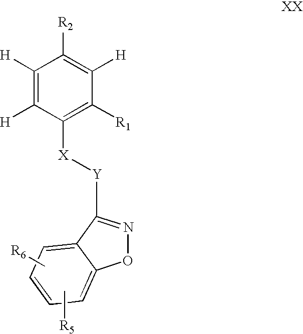
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is H, halo, NO₂, CN, —(CH₂)ₖ-S(O)ᵢ-R₇, —NH—SO₂-R₇, —(CH₂)ₖ-W-R₈—NH(CZ₁)—R₈, —(CZ₁)—NH—R₈, —NH—(CZ₁)—NR₈R_{8,}—(CH₂)ₖ-NR₈R₈, substituted aryl, substituted HET, substituted C₁₋₄alkyl, or substituted Ci4alkenyl;
R₆ is selected from H, halo, aryl, substituted aryl, HET, substituted HET, —CN, NH₂, NO₂, alkyl, substituted alkyl, alkoxy, substituted alkoxy, CH₂)ₖ-S(O)ᵢ-R₇, —NHSO₂-R₇, —(CH₂)ₖ-W-R₈, —NH—(CZ₁)-R_{8,}—(CZ₁)—NH—R₈, —NH—(CZ₁)—NR₈R₈, or substituted C₁₋₄ alkenyl;
R₇ is selected from alkyl, substituted alkyl, aryl, substituted aryl, —N(QI₅)₂, HET, and substituted HET;
Each R₈ is independently H, alkyl, substituted alkyl, —OQ₁₆, aryl, substituted aryl, HET, substituted HET, cycloalkyl, and substituted cycloalkyl, or two R₈ substituents when attached to the same atom may be taken together to form a 5-8 membered ring, wherein the ring includes the atom to which the two Rs substituents attach;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ1₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O —C(O)Q₁₆, —C(S)Q₁₆, —C(O)OQ₁₆, —OC(O)Q₁₆, —C(O)NQ₁₆Q₁₆, —C(S)NQ₁₆Q₁₆, —(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O) —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, cycloalkyl, phenyl, benzyl, CH₂-substituted phenyl, and Het in which each of alkyl, cycloalkyl, phenyl, and Het optionally include 1-3 halos;
W is O, S, —(CZ₂)—, or —(CHZ₃)—, provided that W is not S or 0 when R₅ or R₆ are —(CH₂)ₖ-W-OR₁₆;
Z₁ is ═O;
Z₂ is ═O, ═S , ═N—OH, ═N—O-alkyl, or ═N—O-substituted alkyl;
Z₃ is —OH, —N═N-alkyl, —NH-alkyl, or —NH-substituted alkyl;
i is 0, 1, or 2; and
k is 0, 1, or 2.

7. The compound of claim 6, wherein at least one of R₅ and R₆ is a substituted phenyl or substituted HET.

8. The compound of claim 7, wherein at least one of R₅ and R₆ is pyridine, pyrimidine, pyridazine, or pyrazine, each of which is optionally substituted with the substituents described for substituted HET.

9. The compound of claim 7, wherein the substituted phenyl has the formula 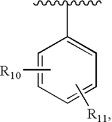
R₁₁, wherein each R₁₀ and R₁₁ is selected from —F, —Cl, —Br, —I, —OQ₁₆, -Q₁₆, —SQ₁₆, —S(O)₂Q₁₆, —S(O)Q₁₆, —OS(O)₂Q₁₆, —SC(O)Q₁₆, —NQ₁₆Q₁₆, ——C(S)Q₁₆, —C(O)OQ₁₆, —OC(O)Q₁₆, —C(O)NQ₁₆Q₁₆, —C(S)NQ₁₆Q₁₆, (O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, NQ₁₆C(S)NQ₁₆Q₁₆, ——S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆.

10. The compound of claim of claim 8, wherein the substituted phenyl has the formula

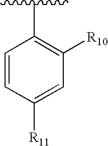

11. The compound of claim 6, wherein one of R₅ or R₆ is —NH—(CZ₁)—NR₈R₈.

12. The compound of claim 1 1, wherein —NR₈R₈ forms a 5-8 membered ring.

13. The compound of claim 12, wherein the ring is morpholino, pyrrolidinyl, or piperdinyl.

14. The compound of claim 11, wherein at least one of the R₈ substituents is benzyl or
—CH₂-substituted phenyl.

15. The compound of claim 6, wherein one of R₅ or R₆ is 4CH₂)ₖ-S(O)ᵢ-R₇ or NH—SO₂—R₇.

16. The compound of claim 15, wherein R₇ is het, substituted het, alkyl, or substituted alkyl.

17. The compound of claim 16, wherein het is indolinyl, pyrrolindinyl, or indolyl, pyrrolyl.

18. The compound of claim 16, wherein sustituted het includes a het substituent substituted with 1-3 of halo or CN.

19. The compound of claim 16, wherein substituted alkyl is an alkyl substituted with 1-3 of OH, NH₂, NHQ₁₆, —NR₈R₈.

20. The compound of claim 1 having a formula XXX 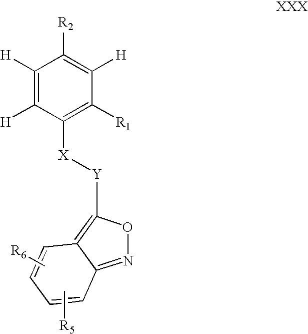
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is H, halo, NO₂, CN, CH₂)ₖ-S(O)ᵢ-R₇, —NH—SO₂-R₇, —(CH₂)ₖ-W-Rs —NH(CZ₁)-R₈, —(CZ₁)—NH—R₈, —NH—(CZ₁)—NR_{8,}—(CH₂)ₖ—NR₈R_{8,} substituted aryl, substituted HET, substituted C₁₋₄alkyl, or substituted C1₄alkenyl;
R₁ is selected from H, halo, aryl, substituted aryl, HET, substituted HET, —CN, NH₂, NO₂, alkyl, substituted alkyl, alkoxy, substituted alkoxy, CH₂)ₖ-S(O)ᵢ-R₇, —NHSO₂—R₇, —(CH₂)ₖ-W-R₈, —NH—(CZ₁)-R_{8,}—(CZ₁)—NH—R₈, —NH—(CZ₁)—NR₈R₈, or substituted C₁₋₄alkenyl;
R₇ is selected from alkyl, substituted alkyl, aryl, substituted aryl, —N(Q₁₅)₂, HET, and substituted HET;
Each R₈ is independently H, alkyl, substituted alkyl, —OQ₁₆, aryl, substituted aryl, HET, substituted HET, cycloalkyl, and substituted cycloalkyl, or two R₈ substituents when attached to the same atom may be taken together to form a 5-8 membered ring, wherein the ring includes the atom to which the two R₈ substituents attach;
Each Qi5 is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ1₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being flirther optionally substituted with ═O or ═S;
Each Q₁₆ is independently selected from —H, alkyl, cycloalkyl, phenyl, benzyl, CH₂-substituted phenyl, and Het in which each of alkyl, cycloalkyl, phenyl, and Het optionally include 1-3 halos;
W is O, S, —(CZ₂)—, or —(CHZ₃)-, provided that W is not S or 0 when R₅ or R₆ are —(CH₂)ₖ-W-OR₁₆;
Z₁ is ═O;
Z₂ is ═O, ═S, ═N—OH, ═N—O-alkyl, or ═N—O-substituted alkyl;
Z₃ is —OH, —N═N-alkyl, —NH-alkyl, or —NH-substituted alkyl;
i is 0, 1, or 2; and
k is 0, 1, or 2.

21. The compound of claim 20, wherein at least one of R₅ and R₆is a substituted phenyl or substituted HET.

22. The compound of claim 21, wherein at least one of R₅ and R₆is pyridine, pyrimidine, pyridazine, or pyrazine, each of which is optionally substituted with the substituents described for substituted HET.

23. The compound of claim 21, wherein the substituted phenyl has the formula 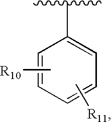
wherein each R₁₀ and R₁₁ is selected from —F, —Cl, —Br, —I, —OQ₁₆, —₁₆, —SQ1₆, —S(O)₂Q₁₆, —S(O)Q₁₆, —OS(O)₂Q₁₆, —SC(O)Q₁₆, —NQ₁₆Q₁₆, ——C(S)Q₁₆, —C(O)OQ₁₆, —OC(O)Q₁₆, —C(O)NQ₁₆Q₁₆, —C(S)NQ₁₆Q₁₆, (O)C(Q₁₆)₂OC(O —CN, —NQ₁₆C(O)Q₁₆, —NQ₁₆C(S)Q₁₆, —NQ₁₆C(O)NQ₁₆Q₁₆, NQ₁₆C(S)Q₁₆, —NQ₁₆C(O)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆.

24. The compound of claim of claim 23, wherein the substituted phenyl has the formula

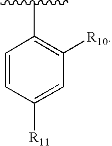

25. The compound of claim 20, wherein one of R₅ or R₆ is —NH—(CZ₁)—NR₈R₈.

26. The compound of claim 25, wherein —NR₈R₈ forms a 5-8 membered ring.

27. The compound of claim 26, wherein the ring is morpholino, pyrtolidinyl, or piperdinyl.

28. The compound of 26, wherein at least one of the R₈ substituents is benzyl or —CH₂-substituted phenyl.

29. The compound of claim 20, wherein one of R₅ or R₆ is dCH₂)ₖ-S(O)ᵢ-R₇ or NH—SO₂—R₇.

30. The compound of claim 29, wherein R₇ is het, substituted het, alkyl, or substituted alkyl.

31. The compound of claim 30, wherein het is indolinyl, pyrrolindinyl, or indolyl, pyrrolyl.

32. The compound of claim 30, wherein sustituted het includes a het substituent substituted with 1-3 of halo or CN.

33. The compound of claim 30, wherein substituted alkyl is an alkyl substituted with 1-3 of OH, NH₂, NHQ₁₆, —NR₈R₈.

34. The compound of claim 1 having a formula VII 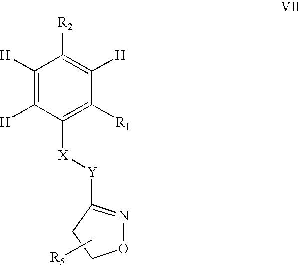
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is 4CH₂)ₖ-S(O)ᵢ-R₇, —NH—SO₂—R₇, —(CH₂)ₖ-W-R₈, —NH—(CZ₁)—R₈, —NH(CZ₁)—R₈, —NH—(CZ₁)—NR₈, substituted aryl, substituted C₁₋₄alyl, or substituted C₁₋₄alkenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(QI₅)₂, HET, and substituted HET;
R₈ is H, alkyl, substituted alkyl, aryl, substituted aryl, HET, substituted HET, cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆Q₁₆, C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆QI_{—S(O)}₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is 0;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i isO, 1, or2; and
k is 0, 1, or 2.

35. The compound of claim 1 having a formula VIII 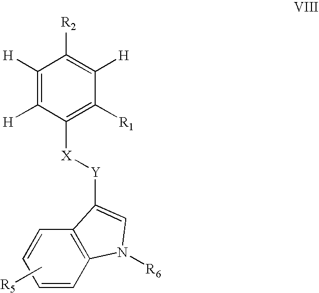
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is —CH₂)ₖ-S(O)ᵢ-R₇, —NH—SO₂—R₇, —(CH₂)ₖ-W-R₈, —NH—(CZ₁)-R₈, —NH(CZ₁)—NR₈, substituted aryl, substituted C₁₋₄alyl, or substituted C₁₋₄alkenyl;
R₆ is selected from H, halo, C₁-C₄ alkyl, —CN, NH₂, NO₂;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, C(═NQ₁₆, —S(O)₂—N═S(O)(Q₁₆)₂, —S(O)₂—N═S(Q₁₆)₂, —SC(O)Q₁₆, —NQ₁₆Q₁₆, C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

36. The compound of claim 1 having a formula IX 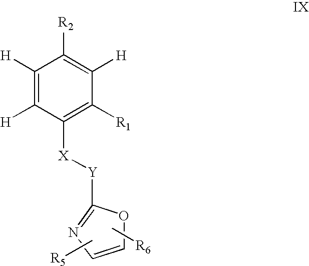
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is CH₂)ₖ-S(O)ᵢ-R₇, —NH—SO₂—R₇, —(CH₂)ₖ-W-R₈, —NH—(CZ₁)—R₈, —NH—(CZ₁)—NR₈, substituted aryl, substituted C₁₋₄alkyl, or substituted C₁₋₄alkenyl;
R₆ is selected from H, halo, —CN, NH₂, NO₂, alkyl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁, is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or2; and
k is 0, 1, or 2.

37. The compound of claim 1 having a formula X 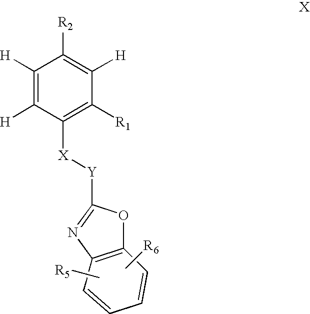
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is CH₂)ₖ-S(O)ᵢ-R_{7,}—NH—SO₂—R₇, —(CH₂)ₖ-W-R₈, —NH—(CZ₁)-R₈, —NH(CZ₁)—NR₈, substituted aryl, substituted C₁₋₄alkyl, or substituted C₁₋₄alkenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Qi5 is independently H, alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ1₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

38. The compound of claim 1 having a formula XI 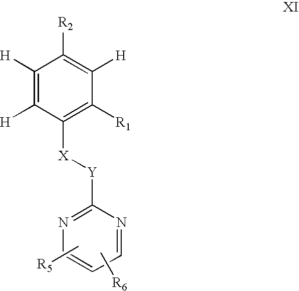
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is CCH₂)ₖ-S(O)ᵢ-R₇, —NH—SO₂—R₇, —(CH₂)ₖ-W-R₈, —NH—(CZl)-R₈, —NH(CZ₁) substituted aryl, substituted C₁₋₄alyl, or substituted C₁₋₄alkenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently H,. alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Qi6, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆,—NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being further optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z, is O;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2; and
k is 0, 1, or 2.

39. The compound of claim 1, wherein Y is —CO—.

40. The compound of claim 1, wherein R₂ is halo, —CN, —NO₂, HET, substituted HET, aryl, substituted aryl, —(CO)-alkyl, —(CO)-substituted alkyl, —(CO)-aryl, —(CO)-substituted aryl, —(CO)-O-alkyl, —(CO)-O-substituted alkyl, —(CO)-O-aryl, —(CO)-O-substituted aryl, —OC(Zₙ)₃, —C(Z)₃, —C(Zₙ)₂-O—C(Zₙ)₃, —SO₂—C(Zₙ)₃, —SO₂-aryl, CN(Q₁₇)₂, —C(NQ₁₇)Q₁₇. —CH═C(Q₁₇)₂, —C≡C—Q₁₇, in which each Zn and Zm is independently H, halo, —CN, —NO₂ —OH, or C₁₋₄alkyl optionally substituted with 1-3 halo, —OH, NO₂, provided that at least one of Zn is halo, —CN, or NO₂.

41. The compound of claim 40, wherein R₂ is Br, Cl, F, I, —CN, formyl, methoxyimnino, hydroxyimino, —CH₂-halo, CH₂—CN, phenyl, thienyl, pyrazinyl, 1-methyl-1H-pyrrol-2-yl, pyridin-2-yl, chlorophenyl, nitrophenyl, cyanophenyl, chlorothienyl, methylthienyl, fluorophenyl, (trifluoromethy)phenyl, di (trifluoromethy)phenyl, difluorophenyl, dimethylisoxazolyl, dimethoxypyrimidinyl.

42. The compound of claim 1, wherein R₅ is —NH₂, —SO₂—NH-alkyl, -SO2—NH-substituted alkyl, -SO2—NH-aryl, —NH—SO2-aryl, -SO₂—NH-substituted aryl, —NH—SO₂-substituted aryl, -SO₂—NH-HET, —SO₂—NH-substituted HET, -SO₂—N(alkyl)(substituted alkyl), -SO₂—N(alkyl)(aryl), -SO₂—N(alkyl)(substituted aryl), -SO₂—N(alkyl)(HET), SO₂—N(alkyl)-(substituted HET), -S-alkyl, -S-substituted alkyl, -0-alkyl, -0-aryl, -Ssubstituted alkyl, —CH₂-S-alkyl, —CH₂-S-substituted alkyl, —(CH₂)₂-S-alkyl, —(CH₂)₂-S-substituted alkyl, —C(O)-aryl, —C(O)H, —C(OH)-aryl, -C(N—OCH₃)-aryl, -C(N—OH)-aryl, —C(O)—C₁₋₆cycloalkyl, —NH—C(O)-O-C1-4alkyl, —NH—C(O)-aryl, —NH—C(O)-substituted aryl, —NH—C(O)-HET, —NH—C(O)-substituted HET, —NHC(O)NH-aryl, —NHC(O)NH-substituted aryl, —NHC(O)NH-het, —NHC(O)NH-substituted het.

43. The compound of claim 42, wherein R₅ is (diethylamino)sulfonyl, (1H-indol-5-yl)aminosulfonyl, (furylmethylamino)sulfonyl, (ethoxycarbonyl)-1-piperazinylsulfonyl, pyridinylethylaminosulfonyl, (benzylamino)sulfonyl, (2-hydroxy-1-methylethyl)aminosulfonyl, (4-carboxyanilino)sulfonyl, (3,4-dihydro-1(2H)quinolinyl)sulfonyl, [2-(3,5-dimethoxyphenyl)ethyl]aminosulfonyl, [(3S)-3 hydroxypyrrolidinyl]sulfonyl, (ethylanilino)sulfonyl, (3,5-dimethoxyanilino)sulfonyl, (2 hydroxy-2-phenylethyl) (methyl)amino]sulfonyl, (2,3-dihydro-1H-indol-1-yl)sulfonyl, (5-methoxy-2,3-dihydro-1H-indol-1-yl)sulfonyl, (5-fluoro-2,3-dihydro-1H-indol-1-yl)sulfonyl, (1H-benzimidazol-1-yl)sulfonyl, (5-fluoro-1H-indol-1-yl)sulfonyl, (1H-indol-1-yl)sulfonyl, (6-fluoro-1H-indol-1-yl)sulfonyl, (5-chloro-1H-indol-1-yl)sulfonyl, (6-chloro-1H-indol-1-yl)sulfonyl, (6-chloro-5-fluoro-1H-indol-1-yl)sulfonyl, (1H-pyrrol-1-yl)sulfonyl, (5-methoxy-1H-indol-1-yl)sulfonyl, (1H-pyrrolo[2,3-b]pyridin-1-yl)sulfonyl, (5-bromo-2,3-dihydro-1H-indol-1-yl)sulfonyl, (3,5-dimethyl-2,3-dihydro-1H-indol-1-yl)sulfonyl, (4-chlorophenyl)(methyl)amino]sulfonyl, benzylthio, methyl(pyridin-2-yl)amimo]sulfonyl, (1H-indol-1-yl)sulfonyl, (pyrrolidin-1-yl)sulfonyl, (2-methylpyrrolidin-1-yl)sulfonyl, (morpholin-4-yl)sulfonyl, (piperidin-1-yl)sulfonyl, (methoxy-1H-indol-1-yl)sulfonyl, {methyl[(1 R)-1-phenylethyl]amino}sulfonyl, {methyl[(1 S)-1-phenylethyl]amino}sulfonyl, [(2-aminophenyl)(methyl)amino]sulfonyl, (dipropylamino)sulfonyl, benzylsulfanyl, (dipropylamino)sulfanyl, (dipropylamino)sulfinyl, [4-chloro(methyl)anilino]sulfonyl, (phenylthio)methyl, benzyloxy, 3-(ethylthio), (pyridin-4-ylmethyl)thio, phenoxy, phenylthio, (pyridin-4-ylmethyl)thio, benzylthio, (1-phenylethyl)thio, cyclopentylthio, cyclopentylsulfinyl, benzoyl, hydroxy(phenyl)methyl, (methoxyinm ino)(phenyl)methyl, (hydroxyimino)(phenyl)methyl, cyclopentylcarbonyl, benzoylamino, furoylamino, (thien-2-ylacetyl)amino, (mesitylcarbonyl)amino, (1,3-benzodioxol-5-ylcarbonyl)amino, 3-(2,4-dimethoxybenzoyl)amino, (phenylthio)acetylamino, (anilinocarbonyl)amino, (2,4-difluorophenyl)amino carbonylamino, (3-cyanophenyl)aminocarbonylamino, (3-acetylphenyl)aminocarbonylamino, (trifluoromethoxy)phenylsulfonylamino, (thien-2-ylacetyl)amino, (5-nitro-2furoyl)amino, (5-chloro-2-methoxyphenyl)aminocarbonylamino, (4phenoxyphenyl)aminocarbonylamino, (4-acetylphenyl)aminocarbonylamino, phenylethynyl, 2-phenylethyl, 4-Chlorophenyl, benzyloxy, phenoxy, alkylthio, phenyl, dihalophenyl, amino, acetylamino, benzoylamino, phenylacetylamino, methylsulfonylamino, phenylsulfonylamino, benzylsulfonylamino, benzyloxy, hydroxy, 3-phenoxypropoxy, (2,3-dihydro-1,4-benzodioxin-2-yl)methoxy, cyclobutylmethoxy, (2,2-dimethyl-1,3-dioxolan-4-yl)methoxy, 2,3-dihydroxypropoxy, cyclobutyloxy, 2-methoxy-1-methylethoxy, isopropoxy, cyclopropylmethoxy, cyclohexylmethoxy, 2-methoxyethoxy, tetrahydro-2H-pyran-2-yl-methoxy, (oxiran-2-yl)methoxy, 2-hydroxy-3-isopropoxypropoxy, furylmethoxy, pentyloxy, phenylacetylamino, Benzoylamino, Acetyloxyacetylamino, cyclopentylcarbonylamino, 6-Chloropyridin-3-ylcarbonylamino, isoxazol-5-ylcarbonylamino, 2,4-difluorobenzoylamino, fluoroacetylamino, Acetylamino, 4-Chlorophenylacetylamino, 4-methoxyphenylacetylamino, cyclopentylacetylamino, 3-fluorobenzoylamino, 3-cyanophenylacetylamino, cyclohexylcarbonylamino, propionylamino, 5-methoxy-5-oxopentanoylamino, Butyrylamino, 4-Bromobenzoylamino, 3-phenylpropanoylamino, phenoxyacetylamino, 5 3-cyclopentylpropanoylamino, 3-methoxy-3-oxopropanoylamino, 2-ethylhexanoylamino, 3,4-dimethoxyphenylacetylamino, 3,5,5-trimethylhexanoylamino, cyclopropylcarbonylamino, methoxyacetylamino, 3-methylbutanoylamino, pentanoylamino, 4,7,7-trimethyl-3-oxo-2-oxabicyclo[2.2.1]hept-1-ylcarbonylanino, Chloro(phenyl)acetylamino, Benzyloxyacetylamino, 3-ethoxy-3-oxopropanoylamino, 1o 1-Adamantylcarbonylamino, hexanoylamino, 2-phenylcyclopranolyamino, 2-phenylbutanoylamino, heptanoylamino, Acetyloxyphenylacetylamino, thien-2-ylcarbonylamino, 2-methylbutanoylamino, 8-methoxy-8-oxooctanoylamino, 2-ethylbutanoylamino, octanoylamino, cyclobutylcarbonylamino, 1,3-dioxo-1,3-dihydro-2H-isoindol-2-yl, Benzylthio, morpholin-4-ylsulfonylbenzoylamino, 1H-indol-2-ylcarbonylamino, 1-methyl-1H-indol-2-ylcarbonylamino, 5-phenylisoxazol-3-ylcarbonylamino, 5-phenylpentanoylamino, 4-phenylbutanoylamino, 4-(4-methoxyphenyl)butanoylamino, 2-Chlorophenylacetylamino, 2,4dichlorophenylacetylamino, 3,4-dichlorophenylacetylamino, 3Chlorophenylacetylamino, 3-(trifluoromethyl)phenylacetylamino, 3-methylphenylacetylamino, 4-tert-Butylphenylacetylamino, 3-methoxyphenylacetylamino, 2-methoxyphenylacetylamino, 2-methylphenylacetylamino, 4-(trifluoromethyl)phenylacetylamino, 4-isopropylphenylacetylamino, 4-methylphenylacetylamino, 4-fluorophenylacetylamino, 2(trifluoromethyl)phenylacetylamino, 3-fluorophenylacetylamino, phenylthioacetylamino, naphthylacetylanino, naphthyloxyacetylamino, 2-propoxybenzoylamino, tetrahydrofuran-3-ylcarbonylamino, 1-methylcyclopropylcarbonylamino, 4-ethoxyphenylacetylamino, 1-Benzothien-3-ylacetylamino, 1,1′-Biphenyl-4-ylcarbonylamino, 4-Butoxybenzoylamino, 2-(2-phenylethyl)benzoylamino, 1,1′-Biphenyl-2-ylcarbonylamino, 4-(ethylthio)benzoylamino, 2-(methylsulfonyl)benzoylamino, 2,6dichlorophenylacetylamino, 1,1′-Biphenyl-4-ylacetylamino, 1,3-Benzodioxol-5-ylacetylamino, 3,3-dimnethylbutanoylamino, thien-2-ylacetylamino, 3-methyl-5-phenylisoxazol-4-ylcarbonylamino, [2-(2-methoxyethoxy)ethoxy]acetylamino, (2-hydroxybenzoyl)amino, prolylamino, (3-methylisoxazol-5-yl)acetylamino, and 4Azido-3-iodobenzoylamino.

44. The compound of claim 1, wherein R₆ is H, halo, —CN, NH₂, NO₂, methyl, methoxy, —(CH₂)₂—OH, morpholinyl, and —(CH₂)₂—O—CO—CH₃.

45. A compound selected from
5-cyano-2-[(1H-indol-2-ylcarbonyl)amino]benzoic acid;
5-cyano-2-{[(5-methoxy-1H-indol-2-yl)carbonyl]amino}benzoic acid;
2-({[5-(benzyloxy)-1H-indol-2-yl]carbonyl}amino)-5-cyanobenzoic acid;
5-cyano-2-{[(1-methyl-1H-indol-2-yl)carbonyl]amino}benzoic acid;
2-({[6-(benzyloxy)-1H-indol-2-yl]carbonyl}amino)-5-cyanobenzoic acid;
2-{[(7-chloro-1H-indol-2-yl)carbonyl]amino}-5-cyanobenzoic acid;
5-cyano-2-{[(4-methoxy-1H-indol-2-yl)carbonyl]amino}benzoic acid;
5-bromo-2-{[(1-methyl-1H-indol-2-yl)carbonyl]amino}benzoic acid;
2-{[(6-chloro-1H-indol-2-yl)carbonyl]amino}-5-cyanobenzoic acid;
2-{[(1-benzyl-1H-indol-2-yl)carbonyl]amino}-5-cyanobenzoic acid;
5-cyano-2-{[(1-ethyl-1H-indol-2-yl)carbonyl]amino}benzoic acid;
5-cyano-2-({[7-(phenylsulfonyl)-1H-indol-2-yl]carbonyl}amino)benzoic acid;
2-{[(1-allyl-1H-indol-2-yl)carbonyl]amino}-5-cyanobenzoic acid;
5-cyano-2-({[1-(cyclohexylmethyl)-1H-indol-2-yl]carbonyl}amino)benzoic acid
{[1-(2-methoxyethyl)-1H-indol-2-yl]carbonyl}amino)benzoic acid;
5-cyano-2-{[(1-pentyl-1H-indol-2-yl)carbonyl]amino}benzoic acid;
2-{[(1-butyl-1H-indol-2-yl)carbonyl]amino}-5-cyanobenzoic acid;
5-cyano-2-{[(1-propyl-1H-indol-2-yl)carbonyl]amino}benzoic acid;
5-chloro-2-{[(1-propyl-1H-indol-2-yl)carbonyl]amino}benzoic acid;
2-{[(1-butyl-1H-indol-2-yl)carbonyl]amino}-5-chlorobenzoic acid;
5-chloro-2-{[(1-pentyl-1H-indol-2-yl)carbonyl]amino}benzoic acid;
5-chloro-2-({[1-(2-methoxyethyl)-1H-indol-2-yl]carbonyl}amino)benzoic acid;
5-chloro-2-({[1-(cyclohexylmethyl)-1H-indol-2-yl]carbonyl}anino)benzoic acid;
2-{[(1-allyl-1H-indol-2-yl)carbonyl]amino}-5-chlorobenzoic acid;
2-{[(1-allyl-1H-indol-2-yl)carbonyl]amino}-5-bromobenzoic acid;
5-bromo-2-({[1-(cyclohexylmethyl)-1H-indol-2-yl]carbonyl}amino)benzoic acid;
5-bromo-2-({[1-(2-methoxyethyl)-1H-indol-2-yl]carbonyl}amino)benzoic acid;
5-bromo-2-{[(1-pentyl-1H-indol-2-yl)carbonyl]amino}benzoic acid;
5-bromo-2-{[(1-butyl-1H-indol-2-yl)carbonyl]amino}benzoic acid;
5-bromo-2-{[(1-propyl-1H-indol-2-yl)carbonyl]amino}benzoic acid;
2-{[(1-benzyl-1H-indol-2-yl)carbonyl]amino}-5-chlorobenzoic acid;
2-{[(1-benzyl-1H-indol-2-yl)carbonyl]amino}-5-bromobenzoic acid;
5-bromo-2-{[(1-isopropyl-1H-indol-2-yl)carbonyl]amino}benzoic acid;
5-cyano-2-{[(1-isopropyl-1H-indol-2-yl)carbonyl]amino}benzoic acid;
5-chloro-2-{[(1-methyl-1H-indol-2-yl)carbonyl]amino}benzoic acid;
5-chloro-2-{[(1-isobutyl-1H-indol-2-yl)carbonyl]amino}benzoic acid;
5-bromo-2-{[(1-isobutyl-1H-indol-2-yl)carbonyl]amino}benzoic acid;
5-cyano-2-{[(1-isobutyl-1H-indol-2-yl)carbonyl]amino}benzoic acid;
5-cyano-2-({[1-(3-phenylpropyl)-1H-indol-2-yl]carbonyl}amino)benzoic acid;
5-chloro-2-({[1-(3-phenylpropyl)-1H-indol-2-yl]carbonyl}amino)benzoic acid;
5-bromo-2-({[1-(3-phenylpropyl)-1H-indol-2-yl]carbonyl}amino)benzoic acid;
5-chloro-2-({[7-(phenylsulfonyl)-1H-indol-2-yl]carbonyl}amino)benzoic acid;
5-bromo-2-({[7-(phenylsulfonyl)-1H-indol-2-yl]carbonyl}amino)benzoic acid;
5-cyano-2-({[1-methyl-7-(phenylsulfonyl)-1H-indol-2-yl]carbonyl}amino)benzoic acid;
5-bromo-2-({[1-methyl-7-(phenylsulfonyl)-1H-indol-2-yl]carbonyl}amino)benzoic acid;
5-chloro-2-({[1-methyl-7-(phenylsulfonyl)-1H-indol-2-yl]carbonyl}amino)benzoic acid;
5-cyano-2-[({7-[(phenylacetyl)amino]-1H-indol-2-yl}carbonyl)amino]benzoic acid;
2-({[7-(benzoylamino)-1H-indol-2-yl]carbonyl}amino)-5-cyanobenzoic acid;
2-{[(7-{[(acetyloxy)acetyl]amino}-1H-indol-2-yl)carbonyl]amino}-5-cyanobenzoic acid;
5-cyano-2-[({7-[(cyclopentylcarbonyl)amino]-1H-indol-2-yl}carbonyl)amino]benzoic acid;
2-{[(7-amino-1H-indol-2-yl)carbonyl]amino}-5-cyanobenzoic acid;
2-{[(7-{[(6-chloropyridin-3-yl)carbonyl]amino}-1H-indol-2-yl)carbonyl]amino}-5-cyanobenzoic acid;
5-cyano-2-[({7-[(isoxazol-5-ylcarbonyl)amino]-1H-indol-2-yl}carbonyl)amriino]benzoic acid;
5-cyano-2-[({7-[(2,4-difluorobenzoyl)amino]-1H-indol-2-yl}carbonyl)amino]benzoic acid;
5-cyano-2-[({7-[(fluoroacetyl)amino]-1H-indol-2-yl}carbonyl)amimo]benzoic acid;
2-({[7-(acetylamino)-1H-indol-2-yl]carbonyl}amino)-5-cyanobenzoic acid;
2-{[(7-{[(4-chlorophenyl)acetyl]amino}-1H-indol-2-yl)carbonyl]amino}-5-cyanobenzoic acid;
5-cyano-2-{[(7-{[(4-methoxyphenyl)acetyl]amino}-1H-indol-2-yl)carbonyl]amino}benzoic acid;
5-cyano-2-[({7-[(cyclopentylacetyl)amino]-1H-indol-2-yl}carbonyl)amino]benzoic acid;
5-cyano-2-[({7-[(3-fluorobenzoyl)amimo]-1H-indol-2-yl}carbonyl)amino]benzoic acid;
5-cyano-2-[({7-[(cyclohexylcarbonyl)amino]-1H-indol-2-yl}carbonyl)amimo]benzoic acid;
5-cyano-2-({[7-(propionylamino)-1H-indol-2-yl]carbonyl}amino)benzoic acid;
5-cyano-2-[({7-[(5-methoxy-5-oxopentanoyl)amino]-1H-indol-2-yl}carbonyl)amino]benzoic acid;
2-({[7-(butyrylamino)-1H-indol-2-yl]carbonyl}anino)-5-cyanobenzoic acid;
2-[({7-[(4-bromobenzoyl)amino]-1H-indol-2-yl}carbonyl)amimo]-5-cyanobenzoic acid;
5-cyano-2-[({7-[(3-phenylpropanoyl)amino]-1H-indol-2-yl}carbonyl)amino]benzoic acid;
5-cyano-2-[({7-[(phenoxyacetyl)amino]-1H-indol-2-yl}carbonyl)amimo]benzoic acid;
5-cyano-2-[({7-[(3-cyclopentylpropanoyl)amino]-1H-indol-2-yl}carbonyl)amino]benzoic acid;
5-cyano-2-[({7-[(3-methoxy-3-oxopropanoyl)amino]-1H-indol-2-yl}carbonyl)amino]benzoic acid;
5-cyano-2-[({7-[(2-ethylhexanoyl)amino]-1H-indol-2-yl}carbonyl)amino]benzoic acid;
5-cyano-2-{[(7-{[(3,4-dimethoxyphenyl)acetyl]amino}-1H-indol-2-yl)carbonyl]amino}benzoic acid;
5-cyano-2-[({7-[(3,5,5-trimethylhexanoyl)amino]-1H-indol-2-yl}carbonyl)amino]benzoic acid;
5-cyano-2-[({7-[(cyclopropylcarbonyl)amino]-1H-indol-2-yl}carbonyl)amino]benzoic acid;
5-cyano-2-[({7-[(methoxyacetyl)amino]-1H-indol-2-yl}carbonyl)amimo]benzoic acid;
5-cyano-2-[({7-[(3-methylbutanoyl)amino]-1H-indol-2-yl}carbonyl)amino]benzoic acid;
5-cyano-2-({[7-(pentanoylamino)-1H-indol-2-yl]carbonyl}amino)benzoic acid;
5-cyano-2-{[(7-{[(4,7,7-trimethyl-3-oxo-2-oxabicyclo[2.2.1]heptyl)carbonyl}amino-1H-indol-2-yl)carbonyl]amino]benzoic acid;
2-{[(7-{[chloro(phenyl)acetyl]amino}-1H-indol-2-yl)carbonyl]amino}-5-cyanobenzoic acid;
2-{[(7-{[(benzyloxy)acetyl]amino}-1H-indol-2-yl)carbonyl]amino}-5-cyanobenzoic acid;
5-cyano-2-[({7-[(3-ethoxy-3-oxopropanoyl)amino]-1H-indol-2-yl}carbonyl)amino]benzoic acid;
2-[({7-[(1-adamantylcarbonyl)amino]-1H-indol-2-yl}carbonyl)amimo]-5-cyanobenzoic acid;
5-cyano-2-({[7-(hexanoylamino)-1H-indol-2-yl]carbonyl}amino)benzoic acid;
5-cyano-2-[({7-[(2-phenylbutanoyl)amino]-1H-indol-2-yl}carbonyl)amino]benzoic acid;
5-cyano-2-({[7-(heptanoylamino)-1H-indol-2-yl]carbonyl}amino)benzoic acid;
2-{[(7-{[(acetyloxy)(phenyl)acetyl]amino}-1H-indol-2-yl)carbonyl]amino}-5-cyanobenzoic acid;
5-cyano-2-{[(7-{[(2-phenylcyclopropyl)carbonyl]amino}-1H-indol-2-yl)carbonyl]amino}benzoic acid;
5-cyano-2-[({7-[(thien-2-ylcarbonyl)amino]-1H-indol-2-yl}carbonyl)amino]benzoic acid;
5-cyano-2-[({7-[(2-methylbutanoyl)amino]-1H-indol-2-yl}carbonyl)amino]benzoic acid;
5-cyano-2-[({7-[(8-methoxy-8-oxooctanoyl)amino]-1H-indol-2-yl}carbonyl)amino]benzoic acid;
5-cyano-2-[({7-[(2-ethylbutanoyl)amino]-1H-indol-2-yl}carbonyl)amino]benzoic acid;
5-cyano-2-({[7-(octanoylamino)-1H-indol-2-yl]carbonyl}amino)benzoic acid;
5-cyano-2-[({7-[(cyclobutylcarbonyl)amino]-1H-indol-2-yl}carbonyl)amino]benzoic acid;
5-cyano-2-({[7-(1,3-dioxo-1,3-dihydro-2H-isoindol-2-yl)-1H-indol-2-yl]carbonyl}amino)benzoic acid;
2-({[7-({[2-(benzylthio)-1,3-thiazol-4-yl]carbonyl}amino)-1H-indol-2-yl]carbonyl}amino)-5-cyanobenzoic acid;
5-cyano-2-{[(7-{[3-(morpholin-4-ylsulfonyl)benzoyl]amino}-1H-indol-2-yl)carbonyl]amino}benzoic acid;
5-cyano-2-[({7-[(1H-indol-2-ylcarbonyl)amino]-1H-indol-2-yl}carbonyl)amino]benzoic acid;
5-cyano-2-{[(7-{[(1-methyl-1H-indol-2-yl)carbonyl]amino}-1H-indol-2-yl)carbonyl]amino}benzoic acid;
5-cyano-2-{[(7-{[(5-phenylisoxazol-3-yl)carbonyl]amino}-1H-indol-2-yl)carbonyl]amino}benzoic
5-cyano-2-[({7-[(5-phenylpentanoyl)amino]-1H-indol-2-yl}carbonyl)amino]benzoic acid;
5-cyano-2-[({7-[(4-phenylbutanoyl)amino]-1H-indol-2-yl}carbonyl)amino]benzoic acid;
5-cyano-2-{[(7-{[4-(4-methoxyphenyl)butanoyl]amino}-1H-indol-2-yl)carbonyl]amino}benzoic acid;
2-{[(7-{[(2-chlorophenyl)acetyl]amino}-1H-indol-2-yl)carbonyl]amino}-5-cyanobenzoic acid;
5-cyano-2-{[(7-{[(2;4-dichlorophenyl)acetyl]amino}-1H-indol-2-yl)carbonyl]amino}benzoic acid;
5-cyano-2-{[(7-{[(3,4-dichlorophenyl)acetyl]amino}-1H-indol-2-yl)carbonyl]amino}benzoic acid;
2-{[(7-{[(3-chlorophenyl)acetyl]amino}-1H-indol-2-yl)carbonyl]amino}-5-cyanobenzoic acid;
5-cyano-2-({[7-({[3-(trifluoromethyl)phenyl]acetyl}anino)-1H-indol-2-yl]carbonyl}amino)benzoic acid;
5-cyano-2-{[(7-{[(3-methylphenyl)acetyl]amino}-1H-indol-2-yl)carbonyl]amino}benzoic acid;
2-{[(7-{[(4-tert-butylphenyl)acetyl]amino}-1H-indol-2-yl)carbonyl]amino}-5-cyanobenzoic acid;
5-cyano-2-{[(7-{[(3-methoxyphenyl)acetyl]amino}-1H-indol-2-yl)carbonyl]amino}benzoic acid;
5-cyano-2-{[(7-{[(2-methoxyphenyl)acetyl]amino}-1H-indol-2-yl)carbonyl]amino}benzoic acid;
5-cyano-2-{[(7-{[(2-methylphenyl)acetyl]amino}-1H-indol-2-yl)carbonyl]amino}benzoic acid;
5-cyano-2-({[7-({[4-(trifluoromethyl)phenyl]acetyl}amino)-1H-indol-2-yl]carbonyl}amino)benzoic acid;
5-cyano-2-{[(7-{[(4-isopropylphenyl)acetyl]amino}-1H-indol-2-yl)carbonyl]amino}benzoic acid;
5-cyano-2-{[(7-{[(4-methylphenyl)acetyl]amino}-1H-indol-2-yl)carbonyl]amino}benzoic acid;
5-cyano-2-{[(7-{[(4-fluorophenyl)acetyl]amino}-1H-indol-2-yl)carbonyl]amino}benzoic acid;
5-cyano-2-({[7-({[2-(trifluoromethyl)phenyl]acetyl}anino)-1H-indol-2-yl]carbonyl}amino)benzoic acid;
5-cyano-2-{[(7-{[(3-fluorophenyl)acetyl]aminol}-1H-indol-2-yl)carbonyl]amino}benzoic acid;
5-cyano-2-{[(7-{[(phenylthio)acetyl]amino}-1H-indol-2-yl)carbonyl]amino}benzoic acid;
5-cyano-2-[({7-[(2-naphthylacetyl)amino]-1H-indol-2-yl}carbonyl)amino]benzoic acid;
5-cyano-2-[({7-[(1-naphthylacetyl)amino]-1H-indol-2-yl}carbonyl)amino]benzoic acid;
5-cyano-2-{[(7-{[(2-naphthyloxy)acetyl]amino}-1H-indol-2-yl)carbonyl]amino}benzoic acid;
5-cyano-2-[({7-[(2-propoxybenzoyl)amino]-1H-indol-2-yl}carbonyl)amino]benzoic acid;
5-cyano-2-[({7-[(tetrahydrofiran-3-ylcarbonyl)amino]-1H-indol-2-yl}carbonyl)amino]benzoic acid;
5-cyano-2-{[(7-{[(1-methylcyclopropyl)carbonyl]amino}-1H-indol-2-yl)carbonyl]amino}benzoic acid;
5-cyano-2-{[(7-{[(4-ethoxyphenyl)acetyl]amino}-1H-indol-2-yl)carbonyl]amino}benzoic acid;
2-[({7-[(1-benzothien-3-ylacetyl)amino]-1H-indol-2-yl}carbonyl)amino]-5-cyanobenzoic acid;
2-[({7-[(1,1 ′-biphenyl-4-ylcarbonyl)amimo]-1H-indol-2-yl}carbonyl)amino]-5-cyanobenzoic acid;
2-[({7-[(4-butoxybenzoyl)amino]-1H-indol-2-yl}carbonyl)arniino]-5-cyanobenzoic acid;
5-cyano-2-{[(7-{[2-(2-phenylethyl)benzoyl]amino}-1H-indol-2-yl)carbonyl]amimo}benzoic acid;
2-[({7-[(1,1 ′-biphenyl-2-ylcarbonyl)amino]-1H-indol-2-yl}carbonyl)amino]-5-cyanobenzoic acid;
5-cyano-2-{[(7-{[4-(ethylthio)benzoyl]aminol}-1H-indol-2-yl)carbonyl]amino}benzoic acid;
5-cyano-2-{[(7-{[2-(methylsulfonyl)benzoyl]amino)}-1H-indol-2-yl)carbonyl]amino}benzoic acid;
5-cyano-2-{[(7-{[(2,6-dichlorophenyl)acetyl]amino}-1H-indol-2-yl)carbonyl]amino}benzoic acid;
2-[({7-[(1,1 ′-biphenyl-4-ylacetyl)amino]-1H-indol-2-yl}carbonyl)amimo]-5-cyanobenzoic acid;
2-[({7-[(1,3-benzodioxol-5-ylacetyl)amino]-1H-indol-2-yl}carbonyl)amino]-5-cyanobenzoic acid;
5-cyano-2-[({7-[(3,5-dimethylbutanoyl)amino]-1H-indol-2-yl}carbonyl)amino]benzoic acid;
5-cyano-2-[({7-[(thien-2-ylacetyl)amino]-1H-indol-2-yl}carbonyl)amino]benzoic acid;
5-cyano-2-{[(7-{[(3-methyl-5-phenylisoxazol-4-yl)carbonyl]amimo}-1H-indol-2-yl) carbonyl]amino}benzoicacid;
5-cyano-2-({[7-({[2-(2-methoxyethoxy)ethoxy]acetyl}amino)-1H-indol-2-yl]carbonyl}amino)benzoic acid;
5-cyano-2-[({7-[(2-hydroxybenzoyl)amino]-1H-indol-2-yl}carbonyl)amino]benzoic acid;
5-cyano-2-({[7-({[4-(trifluoromethoxy)phenyl]sulfonyl}amino)-1H-indol-2-yl]carbonyl}amino)benzoic acid;
5-cyano-2-({[7-(prolylamino)-1H-indol-2-yl]carbonyl}amino)benzoic acid;
5-cyano-2-{[(7-{[(3-methylisoxazol-5-yl)acetyl]amino}-1H-indol-2-yl)carbonyl]amino}benzoic acid;
2-[({7-[(benzylsulfonyl)amino]-1H-indol-2-yl}carbonyl)amino]-5-cyanobenzoic acid;
5-cyano-2-{[(1-methyl-7-{[3-(morpholin-4-ylsulfonyl)benzoyl]amino}-1H-indol-2-yl)carbonyl]amino}benzoic acid;
5-cyano-2-{[(7-{[(4-fluorophenyl)acetyl]amino}-1-methyl-1H-indol-2-yl)carbonyl]amino}benzoic acid;
5-cyano-2-[({7-[(fluoroacetyl)amino]-1-methyl-1H-indol-2-yl}carbonyl)amino]benzoic acid;
5-cyano-2-{[(1-methyl-7-{[(1-methyl-1H-indol-2-yl)carbonyl]amino}-1H-indol-2-yl)carbonyl]amino}benzoic acid;
2-({[6-(benzyloxy)-1-methyl-1H-indol-2-yl]carbonyl}amino)-5-cyanobenzoic acid;
5-cyano-2-{[(6-methoxy-1-methyl-1H-indol-2-yl)carbonyl]amino}benzoic acid;
5-cyano-2-[({1-methyl-7-[(morpholin-4-ylcarbonyl)amino]-1H-indol-2-yl}carbonyl)amino]benzoic acid;
5-cyano-2-({[1-methyl-7-({[(tetrahydrofuran-2-ylmethyl)amimo]carbonyl}amino)-1H-indol-2-yl]carbonly}amino)benzoic acid;
5-cyano-2-{[(7-hydroxy-1-methyl-1H-indol-2-yl)carbonyl]amino}benzoic acid;
2-{[(7-{[(benzylamino)carbonyl]amimo}-1-methyl-1H-indol-2-yl)carbonyl]amino}-5-cyanobenzoic acid;
5-cyano-2-({[7-({[(2,3-dihydroxypropyl)amino]carbonyl}amino)-1-methyl-1H-indol-2-yl]carbonyl}amino)benzoic acid;
1-[{[(2-{[(2-carboxy-4-cyanophenyl)amino]carbonyl}-1-methyl-1H-indol-7-yl)amino]carbonyl}(methyl)amino]benzoic (methyl)amino]-1-deoxyhexitol;
5-cyano-2-({[7-(2,3-dihydro-1,4-benzodioxin-2-ylmethoxy)-1-methyl-1H-indol-2-yl]carbonyl}amino)benzoic acid;
2-({[7-(benzyloxy)-1H-indol-2-yl]carbonyl}amino)-5-cyanobenzoic acid;
5-cyano-2-({([1-methyl-7-(3-phenoxypropoxy)-1H-indol-2-yl]carbonyl}amino)benzoic acid;
5-cyano-2-({[7-(cyclobutylmethoxy)-1-methyl-1H-indol-2-yl]carbonyl}amino)benzoic acid;
5-cyano-2-({[7-(2-furylmethoxy)-1-methyl-1H-indol-2-yl]carbonyl}amino)benzoic acid;
5-cyano-2-{[(7-{[(4S)-2,2-dimethyl-1,3-dioxolan-4-yl]methoxy}-1-methyl-1H-indol-2-y6l)carbonyl]amino}benzoic acid;
5-cyano-2-{[(7-{[(2R)-2,3-dihydroxypropyl]oxy}—1-methyl-1H-indol-2-yl)carbonyl]amino}benzoic acid;
5-cyano-2-({[7-(cyclobutyloxy)-1-methyl-1H-indol-2-yl]carbonyl}amino)benzoic acid;
5-cyano-2-({[7-(2-methoxy-1-methylethoxy)-1-methyl-1H-indol-2-yl]carbonyl}amino)benzoic acid;
5-cyano-2-{[(7-isopropoxy-1-methyl-1H-indol-2-yl)carbonyl]amino}benzoic acid;
2-({[7-(benzyloxy)-1-methyl-1H-indol-2-yl]carbonyl}amino)-5-cyanobenzoic acid;
2-{[(6-sec-butoxy-1-methyl-1H-indol-2-yl)carbonyl]amino}-5-cyanobenzoic acid;
2-{[(6-butoxy-1-methyl-1H-indol-2-yl)carbonyl]amino}-5-cyanobenzoic acid;
5-cyano-2-({[7-(cyclohexylmethoxy)-1-methyl-1H-indol-2-yl]carbonyl}anino)benzoic acid;
5-cyano-2-({[7-(cyclopropylmethoxy)-1-methyl-1H-indol-2-yl]carbonyl}amino)benzoic acid;
5-cyano-2-({[1-methyl-7-(tetrahydro-2H-pyran-2-ylmethoxy)-1H-indol-2-yl]carbonyl}amino)benzoic acid;
5-cyano-2-({[1-methyl-7-(pentyloxy)-1H-indol-2-yl]carbonyl}amino)benzoic acid;
5-cyano-2-({[7-(2-methoxyethoxy)-1-methyl-1H-indol-2-yl]carbonyl}amino)benzoic acid;
5-cyano-2-({[7-(2-hydroxy-3-isopropoxypropoxy)-1-methyl-1H-indol-2-yl]carbonyl}amino)benzoic acid;
5-cyano-2-[({1-methyl-7-[2-(methylthio)ethoxy]-1H-indol-2-yl}carbonyl)amino]benzoic acid;
2-[({7-[(4-azido-3-iodobenzoyl)amino]-1-methyl-1H-indol-2-yl}carbonyl)amino]-5-cyanobenzoic acid;
5-cyano-2-[({7-[(3-cyanobenzoyl)amino]-1H-indol-2-yl}carbonyl)amino]benzoic acid;
5-cyano-2-[({1-methyl-6-[2-(trifluoromethyl)phenyl]-1H-indol-2-yl}carbonyl)amino]benzoic acid;
5-cyano-2-({[1-methyl-6-(2,3,4-trimethoxyphenyl)-1H-indol-2-yl]carbonyl}amino)benzoic acid;
5-iodo-2-{[(i -methyl-1H-indol-2-yl)carbonyl]amino}benzoic acid;
2-({[4-(benzylsulfanyl)-2-pyridinyl]carbonyl}amino)-5-bromobenzoic acid;
2-({[6-(benzylsulfanyl)-2-pyridinyl]carbonyl}anino)-5-bromobenzoic acid;
5-bromo-2-({[3-chloro-5-(trifluoromethyl)-2-pyridinyl]carbonyl}amino)benzoic acid;
5-bromo-2-[(pyridin-2-ylcarbonyl)amino]benzoic acid;
5-bromo-2-{[(5-butylpyridin-2-yl)carbonyl]amino}benzoic acid;
5-bromo-2-[(quinolin-2-ylcarbonyl)amino]benzoic acid;
5-bromo-2-{[(6-bromopyridin-2-yl)carbonyl]amino}benzoic acid;
2-{[(3-benzoylpyridin-2-yl)carbonyl]amino}-5-bromobenzoic acid;
2-{[(6-bromopyridin-2-yl)carbonyl]amino}-5-cyanobenzoic acid;
5-cyano-2-[(pyridin-2-ylcarbonyl)amino]benzoic acid;
5-cyano-2-[(quinolin-2-ylcarbonyl)amino]benzoic acid;
5-cyano-2-{[(2-phenylfuro[2,3-c]pyridin-5-yl)carbonyl]amino}benzoic acid;
5-cyano-2-{[(3-methylfuro[2,3-c]pyridin-5-yl)carbonyl]amino}benzoic acid;
2-({[4-(benzyloxy)pyridin-2-yl]carbonyl}anino)-5-bromobenzoic acid;
5-bromo-2-{[(4-chloro-1-oxidopyridin-2-yl)carbonyl]amino}benzoic acid;
2-({[4-(benzyloxy)pyridin-2-yl]carbonyl}amino)-5-cyanobenzoic acid;
2-({[4-(benzyloxy)-1-oxidopyridin-2-yl]carbonyl}amino)-5-bromobenzoic acid;
2-({[4-(benzylthio)-1-oxidopyridin-2-yl]carbonyl}anino)-5-bromobenzoic acid;
5-cyano-2-[(isoquinolin-3-ylcarbonyl)amino]benzoic acid;
5-bromo-2-[(quinoxalin-2-ylcarbonyl)amino]benzoic acid;
5-bromo-2-{[(5-methylpyrazin-2-yl)carbonyl]amino}benzoic acid;
5-cyano-2-[(pyrazin-2-ylcarbonyl)amino]benzoic acid;
2-({[5-(benzylthio)pyrazin-2-yl]carbonyl}amino)-5-cyanobenzoic acid;
2-({[5-(benzylthio)pyrazin-2-yl]carbonyl}anino)-5-bromobenzoic acid;
2-({[6-(benzylthio)pyrazin-2-yl]carbonyl}amino)-5-cyanobenzoic acid;
2-({[6-(benzylthio)pyrazin-2-yl]carbonyl}amino)-5-bromobenzoic acid;
2-({[5-(butylthio)pyrazin-2-yl]carbonyl}amino)-5-cyanobenzoic acid;
5-bromo-2-({[5-(sec-butylthio)pyrazin-2-yl]carbonyl}amino)benzoic acid;
5-bromo-2-({[5-(butylthio)pyrazin-2-yl]carbonyl}amino)benzoic acid;
2-({[5-(butylthio)pyrazin-2-yl]carbonyl}amimo)-5-chlorobenzoic acid;
5-bromo-2-({[5-(pentylthio)pyrazin-2-yl]carbonyl}amino)benzoic acid;
5-bromo-2-({[5-(hexylthio)pyrazin-2-yl]carbonyl}amino)benzoic acid;
2-({[5-(sec-butylthio)pyrazin-2-yl]carbonyl}amino)-5-cyanobenzoic acid;
5-cyano-2-({[5-(pentylthio)pyrazin-2-yl]carbonyl}amino)benzoic acid;
5-cyano-2-{[(5-{[3-(2-methoxyethoxy)propyl]thio}pyrazin-2-yl)carbonyl]amino}benzoic acid;
5-chloro-2-({[5-(pentylthio)pyrazin-2-yl]carbonyl}amino)benzoic acid;
5-cyano-2-({[5-(hexylthio)pyrazin-2-yl]carbonyl}amino)benzoic acid;
5-chloro-2-({[5-(hexylthio)pyrazin-2-yl]carbonyl}amino)benzoic acid;
2-({[5-(sec-butylthio)pyrazin-2-yl]carbonyl}amino)-5-chlorobenzoic acid;
5-bromo-2-{[(5-methoxypyrazin-2-yl)carbonyl]amino}benzoic acid;
5-cyano-2-({[5-(2-phenylethyl)pyrazin-2-yl]carbonyl}amino)benzoic acid;
5-bromo-2-{[(5-{(E)-2-[(4S)-2,2-dimethyl-1,3-dioxolan-4-yl]ethenyl}pyrazin-2-yl)carbonyl]amino}benzoic acid;
5-cyano-2-({[5-(isopentylthio)pyrazin-2-yl]carbonyl}amino)benzoic acid;
5-cyano-2-({[5-(isobutylthio)pyrazin-2-yl]carbonyl}amino)benzoic acid;
5-cyano-2-{[(5-methoxypyrazin-2-yl)carbonyl]amino}benzoic acid;
5-cyano-2-({[5-(hexyloxy)pyrazin-2-yl]carbonyl}amino)benzoic acid;
5-cyano-2-[({5-[2-(trifluoromethyl)phenyl]pyrazin-2-yl}carbonyl)amino]benzoic acid;
5-cyano-2-[({5-[(4-methoxybenzyl)thio]pyrazin-2-yl}carbonyl)amino]benzoic acid;
5-cyano-2-({[5-(2-fluorophenyl)pyrazin-2-yl]carbonyl}amino)benzoic acid;
5-bromo-2-{[(5-{(E)-2-[(2S)-1,4-dioxaspiro[4.5]dec-2-yl]ethenyl}pyrazin-2-yl)carbonyl]amino}benzoic acid;
5-cyano-2-({[5-(2-methylphenyl)pyrazin-2-yl]carbonyl}amino)benzoic acid;
5-cyano-2-({[5-(2,3,4-trimethoxyphenyl)pyrazin-2-yl]carbonyl}amino)benzoic acid;
5-cyano-2-({[5-(nonylthio)pyrazin-2-yl]carbonyl}amino)benzoic acid;
5-cyano-2-({[5-(octylthio)pyrazin-2-yl]carbonyl}amino)benzoic acid;
5-cyano-2-({[5-(6-methoxypyridin-3-yl)pyrazin-2-yl]carbonyl}anino)benzoic acid;
5-cyano-2-{[(5-phenylpyrazin-2-yl)carbonyl]amino}benzoic acid;
5-cyano-2-[({5-[4-(methylsulfonyl)phenyl]pyrazin-2-yl}carbonyl)amino]benzoic acid;
5-cyano-2-({[5-(3,5-dimethylisoxazol-4-yl)pyrazin-2-yl]carbonyl}amino)benzoic acid;
5-cyano-2-({[6-(hexylthio)pyridazin-3-yl]carbonyl}amino)benzoic acid; and
5-cyano-2-[({6-[2-(trifluoromethyl)phenyl]pyridazin-3-yl}carbonyl)amino]benzoic acid.

46. A method for the sanitizing or disinfecting including administrating an effective amount of the antimicrobial compounds of claim 1.
